(19) United States
(12) Patent Application Publication
(10) Pub. No.: US 2024/0360474 A1
(43) Pub. Date: Oct. 31, 2024

(54) SELF-INACTIVATING VECTORS FOR GENE EDITING

(71) Applicant: Scribe Therapeutics Inc., Alameda, CA (US)

(72) Inventors: Katherine BANEY, Berkeley, CA (US); Isabel COLIN, Oakland, CA (US); Cécile FORTUNY, Oakland, CA (US); Sean HIGGINS, Alameda, CA (US); Suraj MAKHIJA, San Francisco, CA (US); Brett T. STAAHL, Tiburon, CA (US); Maroof ADIL, Davis, CA (US); Benjamin OAKES, El Cerrito, CA (US); Addison WRIGHT, El Cerrito, CA (US); Angus SIDORE, Oakland, CA (US); Manuel MOHR, Berkeley, CA (US); Sarah DENNY, San Francisco, CA (US)

(21) Appl. No.: 18/608,127

(22) Filed: Mar. 18, 2024

Related U.S. Application Data
(63) Continuation of application No. PCT/US2022/076980, filed on Sep. 23, 2022.
(60) Provisional application No. 63/349,025, filed on Jun. 3, 2022.
(60) Provisional application No. 63/247,573, filed on Sep. 23, 2021.

(51) Int. Cl. C12N 15/86; C12N 9/22; C12N 15/11

(52) U.S. Cl. CPC C12N 15/86; C12N 9/22; C12N 15/111; C07K 2319/09; C12N 2310/14; C12N 2310/20; C12N 2310/531; C12N 2750/14122; C12N 2750/14123; C12N 2750/14143; C12N 2750/14152

(57) ABSTRACT

Provided herein are compositions and methods for use of self-inactivating recombinant vectors (SIRV) encoding Class 2 Type V and guide ribonucleic acid (gRNA) sequences useful for nucleic acid sequence editing, and including self-inactivating components. The SIRV may be delivered to cells as part of an AAV vector to target a gene of interest.

### CROSS REFERENCE TO RELATED APPLICATIONS

This application claims priority to U.S. provisional patent application Nos. 63/247,573, filed Sep. 23, 2021, and 64/349,025, filed Jun. 3, 2022, the contents of which are incorporated herein by reference in their entirety.

### INCORPORATION BY REFERENCE OF SEQUENCE LISTING

This application contains an electronic sequence listing. The contents of the electronic sequence listing (SCRB_033_02WO_SeqList_ST26.xml; Size: 3,874,654 bytes; and Date of Creation: Sep. 20, 2022) are herein incorporated by reference in their entirety.

### BACKGROUND

Gene editing holds great promise for treating or preventing many genetic diseases. However, safe and targeted delivery of CRISPR gene editing machinery into the desired cells is necessary to achieve therapeutic benefit. Use of viral vectors, such as adeno-associated viral (AAV) vectors, have shown promise in the delivery of CRISPR components into cells for editing of target nucleic acids, particularly with the choice of the proper serotype (Kotterman M A, et al. Viral Vectors for Gene Therapy: Translational and Clinical Outlook. Annu. Rev. Biomed. Eng. 17:63-89 (2015)). However, the long-term expression of CRISPR nucleases mediated by AAV in the post-mitotic cells raises concerns with specificity, immunogenicity and safety. Thus, there remains a need in the art for compositions and methods for delivering CRISPR gene editing machinery to cells that are self-inactivating after the desired on-target editing of a nucleic acid in the cell takes place.

### SUMMARY

The present disclosure relates to self-inactivating recombinant vectors (SIRV) and self-inactivating adeno-associated virus (siAAV) vectors for the delivery of CRISPR nucleases and guide RNAs to cells for the modification of target nucleic acids. The SIRV disclosed herein can temporally control the expression of one or more of the CRISPR components relative to editing or modification of the target nucleic acid.

As provided herein, self-inactivating recombinant vectors (SIRV) express guide RNAs and Class 2 Type V CRISPR nucleases having a single RNA-guided RuvC domain for genetic editing of a target nucleic acid in target cells and/or tissues, wherein the expression of one or more of the CRISPR components is diminished or eliminated by the self-editing components following editing of the target nucleic acid. The timing of diminishing or eliminating the expression of the one or more CRISPR components relative to editing the target nucleic acid is controlled by the design of the SIRV. A number of design approaches to effect the self-inactivating feature of the constructs are disclosed herein. These approaches and their features can also be used in combination.

The self-inactivating features of the SIRV disclosed herein, when incorporated into a viral vector (e.g., an siAAV) or lipid nanoparticle, confer enhanced safety and a higher degree of specificity to the compositions when utilized for gene editing in a subject compared to systems not employing the self-inactivating features. As described herein, the cleavage of self-inactivating segments (in double-stranded episomal form in cells transduced by the siAAV) by RNPs of the CRISPR nuclease and guide RNA results in reduced or eliminated expression of one of more components of the transgene. As a result of the self-inactivating properties, the SIRV and siAAV have an enhanced safety profile when used to modify a target nucleic acid in a population of cells of a subject.

In another aspect, the disclosure relates to polynucleotide compositions that are designed to prevent the premature degradation of the components encoded by the transgene of the siAAV in a packaging cell during the production of the SIRV and siAAV.

The present disclosure also provides methods for treating a subject having an underlying disorder or disease with the SIRV and siAAV compositions.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows a schematic of the AAV construct described in Example 1.

FIG. 2 is a graph showing results of an editing assay using AAV transgene plasmids nucleofected into mouse neural progenitor cells (mNPCs), as described in Example 1, demonstrating that the CasX and targeting guide in three different vectors (constructs AAV1, AAV2, and AAV3) edits on target (tdTomato) with high efficiency compared to non-targeting control (NT). Editing was assessed by fluorescence activated cell sorting (FACS) 5 days post-transfection. Data are presented as mean±SEM for n=3 replicates. % dtTom: percent of mNPCs expressing tdTomato, as assessed by FACS.

FIG. 3 is a graph showing results of an editing assay using AAV transgene plasmids nucleofected into mNPCs at four different dose levels, as described in Example 1. CasX delivered as an AAV transgene plasmid to mNPCs edited on target with high efficiency in a dose-dependent manner, compared to a non-targeting control (NT). CasX variant 491 with scaffold variant 174 (SEQ ID NO: 2238) and a spacer targeting tdTomato in three different vectors (constructs AAV1, AAV2, and AAV3) were nucleofected in mNPCs, and editing was assessed by FACS 5 days post-transfection. Data are presented as mean±SEM for n=3 replicates.

FIG. 4 is a graph showing results of an editing assay using AAV vector construct 3 transduced into mNPCs at 3-fold dilutions, and assessed by FACS five days post-transduction, as described in Example 1. Data are presented as mean±SEM for n=3 replicates. MOI: multiplicity of infection.

FIG. 5 is a scanning transmission micrograph showing AAV particles with packaged CasX variant 438, gRNA scaffold 174 and spacer 12.7, as described in Example 2. AAV were negatively stained with 1% uranyl acetate. Empty particles are identified by a dark electron dense circle at the center of the capsid.

FIG. 6 shows results of immunohistochemistry staining of mouse coronal brain sections, as described in Example 3. Mice received an intracerebroventricular (ICV) injection of 1×10¹¹ AAV packaged with CasX 491, and gRNA scaffold 174 with spacer 12.7 (top panel), which were able to edit the tdTom locus in the Ai9 mice (edited cells appear white). The bottom panel shows that CasX 491 and scaffold 174 with a non-targeting spacer administered as an AAV ICV injection did not edit at the tdTom locus. Tissues were processed for immunohistochemical analysis 1 month post-injection.

FIG. 7A shows results of an immunohistochemistry staining of a mouse liver section showing that CasX 491 and scaffold 174 with spacer 12.7 administered as an AAV IV injection was able to edit the tdTom locus in vivo in Ai9 mice, as described in Example 3.

FIG. 7B shows results of an immunohistochemistry staining of a mouse heart section showing that CasX 491 and scaffold 174 with spacer 12.7 administered as an AAV IV injection was able to edit the tdTom locus in vivo in Ai9 mice, as described in Example 3. The images are representative of n=3 animals.

FIG. 8 is a graph showing the results of an editing assay of the tdTom locus in mNPCs using AAV transgene plasmids of constructs having variations in the CasX promoters, as described in Example 4. Editing was assessed by FACS 5 days post-transfection. Data are presented as mean±SEM for n=3 replicates.

FIG. 9 is a graph showing the results of an editing assay of the tdTom locus in mNPCs using AAV transgene plasmids of constructs having variations in the CasX promoters, as described in Example 4. Editing was assessed by FACS 5 days post-transfection. Data are presented as mean±SEM for n=3 replicates.

FIG. 10 is a plot and a table that show the results of an editing assay of the tdTom locus in mNPCs using AAV transgene plasmids of constructs having variations in the CasX promoters and transgene size (see table, bottom), as described in Example 4. Editing was assessed by FACS 5 days post-transfection. Data are presented as mean±SEM for n=3 replicates.

FIG. 11 is a pair of graphs that show the results of an editing assay of the tdTom locus in mNPCs using AAV vectors incorporating the same promoters as shown in FIG. 10, as described in Example 4. The graph on the left shows results testing 3-fold dilutions of the constructs, while the graph on the right shows results of editing using an MOI of 2×10⁵ vg/cell. Editing was assessed by FACS 5 days post-transfection. Data are presented as mean±SEM for n=3 replicates.

FIG. 12 is a graph showing the results of an editing assay of the tdTom locus in mNPCs using AAV vectors with protein promoter variants designed to reduce transgene size, compared to AAV with the top 4 protein promoter variants identified previously (AAV.3, AAV.4, AAV.5 and AAV.6), as described in Example 4. Editing was assessed by FACS 5 days post-transfection. Data are presented as mean±SEM for n=3 replicates. The dashed line shows editing levels of AAV.4, the AAV construct that in this experiment was used as a baseline for comparison across the variants.

FIG. 13 is a graph of percent editing versus transgene size (from ITR to ITR, in bp) for all constructs having the varying promoters tested in Example 4. Constructs circled with dashes were identified as having above average editing while minimizing transgene size. The dashed line shows editing levels of AAV.4, the AAV construct that in this experiment was used as a baseline for comparison across variants.

FIG. 14 is a graph showing the results of an editing assay of mNPCs using AAV transgene plasmids having variations in gRNA promoter strength, as described in Example 5. Editing was assessed by FACS 5 days post-transfection. Data are presented as mean±SEM for n=3 replicates.

FIG. 15 is a pair of graphs that show the results of an editing assay of mNPCs using three different AAV vectors having variations in gRNA promoter strength, as described in Example 5. The graph on the left shows results testing 3-fold dilutions of the constructs ranging from 1×10⁴ to 5×10⁵ vg/cell, while the graph on the right shows results of editing using an MOI of 3×10⁵ vg/cell. Editing was assessed by FACS 5 days post-transfection. Data are presented as mean±SEM for n=3 replicates.

FIG. 16 is a bar graph that shows percent editing of the tdTom locus in mNPCs in an experiment to assess use of truncated U6 RNA promoters in constructs when delivered in AAV transgene plasmids designed to minimize the footprint of the Pol III promoter in the delivered transgene, as described in Example 5. Editing was assessed by FACS 5 days post-transfection. Data are presented as mean±SEM for n=3 replicates.

FIG. 17 is a bar graph that shows percent editing of the tdTom locus in mNPCs comparing base construct AAV53 to construct AAV85, when delivered as AAV vector designed to minimize the footprint of the Pol III promoter in the delivered transgene, as described in Example 5.

FIG. 18 is a bar graph that shows editing results of the tdTom locus in an experiment to assess the effects of constructs having engineered U6 RNA promoters when delivered to mNPCs in an AAV vector designed to minimize the footprint of the Pol III promoter in the AAV transgene, as described in Example 5. Editing was assessed by FACS 5 days post-transfection. Data are presented as mean±SEM for n=3 replicates.

FIG. 19 is a scatter plot depicting transgene size (from ITR to ITR, in bp) of all AAV variants tested having engineered U6 RNA promoters on the X-axis vs. percent of mNPCs edited on the Y-axis, as described in Example 5. The dashed line indicates construct AAV53, the construct which had the largest promoter tested, while the dotted line indicates construct AAV89, the construct which had the smallest promoter tested.

FIG. 20 is a graph showing the results of an editing assay of the tdTom locus in mNPCs in an experiment to assess the effects of constructs having engineered Pol III RNA promoters when delivered in an AAV vector designed to minimize the footprint of the Pol III promoter in the AAV transgene, as described in Example 5. Editing was assessed by FACS 5 days post-transfection. Data are presented as mean±SEM for n=3 replicates.

FIG. 21 is a bar graph showing AAV-mediated editing level in mNPCs at an MOI of 3.0E+5 vg/cell using the indicated constructs, as described in Example 5.

FIG. 22 is a bar graph showing editing results of the tdTomato locus in an experiment to assess the effects of AAV constructs having engineered Pol III promoter hybrid variants when delivered to mNPCs in an AAV vector, as described in Example 5. Editing was assessed by FACS five days post-nucleofection.

FIG. 23 is a scatter plot depicting the transgene size (from ITR to ITR, in bp) of all variants tested on the X-axis vs. the percent of mNPCs edited on the Y-axis, as described in Example 5.

FIG. 24 is a graph showing the results of an editing assay of the tdTom locus in mNPCs using AAV transgene plasmids having variations in poly(A) signals, as described in Example 6. Data are presented as mean±SEM for n=3 replicates.

FIG. 25 is a graph showing the results of an editing assay of the tdTom locus in mNPCs using two AAV vectors having the high-performing poly(A) signals, as described in Example 6. Editing was assessed by FACS 5 days post-transfection. Data are presented as mean±SEM for n=3 replicates.

FIG. 26 is a diagram depicting schematics of AAV plasmid constructs containing guide RNA transcriptional units (gRNA scaffold-spacer driven by a U6 promoter) in different orientations in regard to the protein promoter transcriptional unit, as described in Example 7. The tapered points depict the orientation of the transcriptional unit for protein or guide RNA.

FIG. 27 is a graph showing the results of an editing assay of the tdTom locus in mNPCs using AAV transgene plasmids having differences in regulatory element orientation, as described in Example 7. Editing was assessed by FACS 5 days post-transfection. Data are presented as mean±SEM for n=3 replicates.

FIG. 28 is a graph showing the results of an editing assay of NPCs using AAV vectors containing guide RNA transcriptional units (gRNA scaffold-spacer driven by a U6 promoter) in different orientations in regard to the protein promoter transcriptional unit, as described in Example 7. The graph on the left shows results testing 3-fold dilutions of the constructs depicted in FIG. 26 ranging from 1×10⁴ to 2×10⁶ vg/cell. The bar graph on the right shows AAV-mediated percent editing in mNPCs at an MOI of 3.0E+5 vg/cell. Editing was assessed by FACS 5 days post-transfection. Data are presented as mean±SEM for n=3 replicates.

FIG. 29 is a bar graph of results of an editing assay of the tdTom locus in mNPCs using AAV transgene plasmid constructs having different post-transcriptional regulatory elements, and as compared to constructs not having post-transcriptional regulatory elements, as described in Example 8. Editing was assessed by FACS 5 days post-transfection. Data are presented as mean±SEM for n=3 replicates.

FIG. 30 is bar graph showing AAV-mediated editing levels (grey bars) of mNPCs at a viral MOI of 3.0E+5 compared to nucleofection-mediated editing using 150 ng of AAV-cis plasmids (dark bars) expressing the CasX protein 491 under the control of select promoters without (constructs AAV4, AAV5, AAV6) or in combination with different post-transcriptional regulatory element sequences (constructs AAV35-AAV37 for base plasmid 4, constructs AAV38-AAV39 for base plasmid 5, and constructs AAV42-AAV43 for base plasmid 6), as described in Example 8. Editing was assessed by FACS 5 days post-transfection. Data are presented as mean±SEM for n=3 replicates.

FIG. 31 is a bar graph showing AAV-mediated editing levels of mNPCs at a viral MOI of 3.0E+5 for constructs under promoters without (constructs AAV58, AAV59, AAV53) or in combination with different post-transcriptional regulatory element sequences (respectively constructs AAV72-AAV74 for base plasmid 58 containing Jet promoter, constructs AAV75-AAV77 for base plasmid 59 containing Jet+USP promoter, and constructs AAV80 and AAV81 for base plasmid 53 containing UbC promoter), as described in Example 8. Editing was assessed by FACS 5 days post-transfection. Data (n=3) are presented as mean±SEM.

FIG. 32 is a scatterplot comparing the transgene size of each construct evaluated (from ITR to ITR, in bp) to AAV-mediated editing levels in mNPCs at a MOI of 3.0e+5 vg/cell, as described in Example 8. The circled data points represent the constructs identified with the highest editing levels of select transgene size. The horizontal grey line shows the editing level of the benchmark vector AAV.53 for comparative purposes. The vertical grey line delimits vectors that are over or under a 4.9 kb transgene size.

FIG. 33 is a violin plot displaying fold-improvement in AAV-mediated editing from the inclusion of the indicated posttranscriptional regulatory element (PTRE) in the transgene plasmid, relative to a transgene with same promoter but no PTRE, indicated by gray dashed line, as described in Example 8.

FIG. 34 is a bar chart showing editing results of constructs with different neuronal enhancers delivered as AAV transgene plasmids to mNPCs, as described in Example 8. The gray lines show editing levels of reference plasmid 64, harboring a CMV enhancer and a core promoter. Editing was assessed by FACS 5 days post-transfection. Data are presented as mean±SEM for n=3 replicates.

FIG. 35 shows the schematics of AAV constructs with alternative gRNA configurations for constructs having two gRNAs, as described in Example 9. The top schematic is architecture 1, while the bottom is architecture 2. The tapered points depict the orientation of the transcriptional unit for CasX protein or gRNA.

FIG. 36 shows the schematics of AAV constructs with additional alternative gRNA configurations for constructs having two gRNAs, as described in Example 9. The tapered points depict the orientation of the transcriptional unit for CasX protein or gRNA.

FIG. 37 shows the schematics of gRNA stack (Pol III promoter, scaffold, spacer) architectures tested with nucleofection and AAV transduction, as described in Example 9. The AAV transgene harbors dual stacks in different orientations, with spacer 12.7, 12.2 and non-targeting (NT) spacer. The tapered points depict the orientation of the transcriptional unit for CasX protein or gRNA.

FIG. 38 is a graph showing the results of an editing assay for AAV constructs having gRNA stacks delivered via plasmid transfection to mNPCs at the two indicated doses, showing constructs with RNA stacks edit with enhanced potency compared to non-targeting control (NT), as described in Example 9. Editing was assessed by FACS 5 days post-transfection. Data are presented as mean±SEM for n=3 replicates.

FIG. 39 is a graph showing the results of an editing assay of mNPCs using AAV transgene plasmid constructs having two gRNAs in different architectures and with different combinations of spacers (see FIG. 35) compared to construct 3 having a single gRNA and to a non-targeting control, as described in Example 9. Editing was assessed by FACS 5 days post-transfection. Data are presented as mean±SEM for n=3 replicates.

FIG. 40 is a graph showing the results of an editing assay of mNPCs using AAV vector constructs 45-48 having two gRNAs in different architectures and with different combinations of spacers (see FIG. 35) compared to construct 3, as described in Example 9. The left panel shows editing results using 3-fold MOI dilutions ranging from 1×10⁴ to 3×10⁵ vg/cell, while the right panel shows editing results at an MOI of 3×10⁵ vg/cell. Editing was assessed by FACS 5 days post-transduction. Data are presented as mean±SEM for n=3 replicates.

FIG. 41 is a bar graph showing percent editing in mNPCs using AAV transgene plasmid constructs with varying 5′ NLS combinations, and with 3′ NLS 1, 8 and 9 in mNPCs, as described in Example 10.

FIG. 42 is a bar graph showing percent editing in mNPCs using AAV vectors with varying 5′ NLS combinations with 3′ NLS in mNPCs, as described in Example 10.

FIG. 43 is a bar graph showing percent editing in mNPCs using AAV vectors with varying NLS combinations, when delivered in a vector also designed to minimize the footprint of Pol III promoter in the transgene, as described in Example 10.

FIG. 44 is a schematic of a self-inactivating recombinant vector (SIRV) transgene design in which the PAM sequence of the self-limiting segment (white box in the schematic) is varied relative to the PAM sequence of the target nucleic acid, as described in Example 12. Within the top boxes, the first nucleotide ‘T’ of the illustrated PAM motif can be swapped with alternative nucleotides A, C, and G to obtain alternative PAM motifs. The black triangle in the top boxes indicates that PAM sequences have “strength” in the order of TTC>ATC>CTC>GTC. The sequence in the top boxes has SEQ ID NO: 4157.

FIG. 45 is a schematic of a SIRV transgene design in which differences in the nucleotides of the self-limiting segment compared to corresponding positions in the targeting sequence of the gRNA are introduced (arrows pointing to certain “N” positions in the boxes on top of the schematic) to reduce the binding affinity of the gRNA to the self-limiting segment compared to the target nucleic acid. The sequence in the white boxes corresponds to SEQ ID NO: 4157.

FIG. 46 is a schematic of a SIRV transgene design in which a second gRNA is incorporated into the transgene (black box in the schematic) that targets the self-limiting segment while the first gRNA (white box at right side of the schematic) targets the target nucleic acid, as described in Example 14. In this design, the second gRNA is “weaker” and is less efficient in promoting editing compared to the first gRNA. The sequence in the top boxes corresponds to SEQ ID NO: 4157.

FIG. 47 is a schematic of a SIRV transgene design in which a second gRNA is incorporated into the transgene (black box in the schematic) that targets the self-limiting segment while the first gRNA (white box at right side of the schematic) targets the target nucleic acid, as described in Example 13. In this design, the second gRNA is under the control of a “weaker” promoter such that transcription is delayed or reduced compared to the transcription of the first gRNA under the control of a “stronger” promoter. The sequence in the top boxes corresponds to SEQ ID NO: 4157.

FIG. 48 is a bar plot of editing efficiency in a PASS assay using CasX 491 protein at four different PAM sequences (TTC, ATC, CTC and GTC), as described in Example 12. Results are presented as the mean and SEM for duplicate samples.

FIG. 49A is a bar graph displaying viral titer yield from production of the indicated AAV vector constructs with 5 different PAM sequences (TTC, CTC, ATC, GTC and GGGG, in AAV. 24, 25, 26, 27, 28 respectively), as described in Example 12. Vector AAV.31 does not contain a self-targeting sequence.

FIG. 49B is a bar-graph showing the fold-change in titer results normalized to that of the control AAV.31 (value of 1.0), as described in Example 12.

FIG. 50A is a bar graph displaying levels of editing (indels) detected in the ssDNA of AAV vectors of the same constructs shown in FIG. 49A, as described in Example 12.

FIG. 50B is a bar graph showing NGS analysis of indel rates performed on different viral fractions during AAV production (production run 1=AAVX column purified, buffer exchanged fraction versus production run 2, production run 3=crude lysate AAV fractions post cell lysis), as described in Example 12. AAV.25 and AAV.27 contained targeted self-limiting segments while AAV.28 contained a non-targeted segment as a negative control.

FIG. 51 is a graph showing the results of editing levels mediated by AAV transgene plasmids 31, 72 and 73 nucleofected into mNPCs at doses of 250 ng and 125 ng, as described in Example 13.

FIG. 52A is a graph of percent editing levels mediated by AAVs 31, 72 and 73 (the X in AAV.X indicates the corresponding cis plasmid #) using 3-fold serial dilution MOIs in mNPCs infected at 4 MOIs ranging from 1.e+4 to 5.0E+5 viral genome/cell. A gRNA scaffold variant targeting the Rosa-tdT locus was under the control of a different RNA promoter, as described in Example 13. Data (n=3) are presented as mean±SEM.

FIG. 52B is a bar graph showing AAV-mediated editing level in mNPCs at an MOI of 3.0E+5 vg/cell comparing the three constructs (AAVs 31, 72 and 73), as described in Example 13.

FIG. 53 is a graph showing the results of an editing assay that assessed the effects of truncated Pol III RNA promoters when delivered in an AAV vector designed to minimize the size of the promoter in the delivered transgene, as described in Example 13. Data are presented as mean±SEM for n=3 replicates.

FIG. 54 is a graph showing the results of an editing assay comparing two constructs delivered in AAV, as described in Example 13.

FIG. 55 is a graph showing the results of an editing assay of the indicated constructs delivered by nucleofection to mNPCs, assessing engineered Pol III RNA promoters designed to minimize the size of the Pol III promoter in the AAV transgene, as described in Example 13. Data are presented as mean±SEM for n=3 replicates.

FIG. 56A is a graph showing editing levels mediated by AAVs (in AAV.X, the X indicates the AAV-cis plasmid number) delivered to mNPCs using 3-fold serial dilution MOIs ranging from 1.e+4 to 5.0E+6 to viral genome/cell, as described in Example 13. Data (n=3) are presented as mean±SD (standard deviation).

FIG. 56B is a bar graph showing AAV-mediated editing level in mNPCs at MOI of 3.0E+5 vg/cell, as described in Example 13.

FIG. 57 is a scatter plot depicting transgene size of the indicated constructs tested on the X-axis vs. percent of NPCs edited on the Y-axis, as described in Example 13.

FIG. 58 is a schematic showing inclusion of a second gRNA (black box between the two Pol III promoters) to target a sequence specific to the transgene (black portions flanking and internal to the box labeled CasX), separate from the therapeutic target sequence, which is targeted by the first gRNA (white box at the right side of the schematic), as described in Example 14. The sequence in the white boxes corresponds to SEQ ID NO: 4157.

FIG. 59 a schematic of different gRNA architectures tested with nucleofection and AAV transduction. In each case, the gRNA scaffold-spacer unit is driven by a U6 Pol III promoter. The transgenes harbor dual gRNAs driven by Pol III in different orientations, with spacer 12.7, 12.2 and non-target spacer NT. The tapered boxes for the protein promoter and Pol III promoters depict orientation of transcription (transcription occurs in the direction of the tapered point).

FIG. 60A is a graph showing editing levels in mNPC cells mediated by AAVs (the X in AAV.X indicates the corresponding AAV-cis plasmid #) delivering dual guide systems at 3-fold serial dilution MOIs, ranging from 1.e+4 to 3.0E+5 viral genome/cell, as described in Example 14. Editing was assessed by FACS 5 days post-transfection. Data (n=3) are presented as mean±SD.

FIG. 60B is a bar graph showing AAV-mediated editing levels in mNPCs at MOI of 3.0E+5 vg/cell, as described in Example 14.

FIG. 61A is a graph showing AAV-mediated editing levels in mNPCs at the MOI (in vg/cell) indicated on the x-axis. mNPCs were infected with AAV vectors expressing CasX protein 491 under combination of the ubiquitous CMV promoter and engineered scaffold variants (174, 229-237, construct ID=31, 40-47) and guide 12.7 under the expression of pol III promoter hU6, as described in Example 14. Frequency of tdT+ cells was assessed by FACS 5 days post-transfection. Data (n=3) are presented as mean values.

FIG. 61B is a graph showing AAV-mediated editing levels in mNPCs at the MOI (in vg/cell) indicated on the x-axis. mNPCs infected with AAV vectors expressing CasX protein 491 under combination of the ubiquitous CMV promoter and engineered scaffold variants (174, 229-237, construct ID=31, 39-47) and guide 12.7 under the expression of pol III promoter hU6, as described in Example 14. Frequency of tdT+ cells was assessed by FACS 5 days post-transfection. Data (n=3) are presented as mean values. Note, construct 39 has scaffold 229.

FIG. 62 is a bar graph displaying fold-change in editing levels for AAV constructs with engineered guide scaffolds (229-237) compared to guide 174 (set to a value of 1; gray dashed line) in cells infected at a 3.0e+5 MOI, as described in Example 14.

FIG. 63 is a western blot of cell lysates of cells transduced with different siAAV and AAV constructs probed with anti-GAPDH and anti-Cas antibodies, as described in Example 15.

FIG. 64 is a bar plot bar showing AAV-mediated editing levels (frequency of tdT+ cells) in mNPCs at a 3.0e+5 vg/cell MOI, as described in Example 16.

FIG. 65 shows western blots demonstrating silencing of CasX expression during AAV production with different shRNA targeting CasX (shRNA 1-12, SEQ ID NOS: 2873-2884 as shown in Table 27), as described in Example 17. Control refers to HEK293T lysate from untreated cells and serves as a control for CasX staining. Construct 29 is the base construct and does not contain any shRNA.

FIG. 66 is a bar plot based on the scan of the western blot in FIG. 65 displaying relative levels of CasX knockdown normalized to levels of expression detected in the no shRNA control (construct 29), as described in Example 17.

FIG. 67 shows western blots demonstrating silencing of CasX expression during AAV production with shRNA8 supplementation, as described in Example 18. Control refers to HEK293T lysate from untreated cells, and serves as a control for CasX staining. The three doses for Construct 17 were added to the production in the following shRNA:transgene ratios—1:1, 2:1, and 3:1. AAV.30 (Lane 1) is a negative control which does not contain any shRNA.

FIG. 68 is a bar graph showing the relative CasX knockdown normalized to CasX expression from FIG. 67. Lane 1 is the negative control, which does not contain any shRNA, as described in Example 18.

FIG. 69 is a bar graph displaying viral titer yield (viral genome per mL) from AAV (AAV.30) and siAAV vectors (AAV.32, 33) with or without shRNA8 supplementation during production. 1:1, 2:1 and 3:1 ratio of shRNA to transgene plasmid during production is represented by the white triangle, as described in Example 18.

FIG. 70A is a bar graph displaying the indel rate detected in the ssDNA of AAV genomes packaged with different dose of shRNA8 supplemented during production (ID #17 refers to shRNA8, dose 1:1, 1:2, 1:3 relative to plasmid AAV.33). The percent of indels detected in a self-inactivating AAV vector (AAV.33) are compared to a non-self-inactivating AAV control (AAV.30), as described in Example 18.

FIG. 70B is a bar graph showing the percent decrease in indels detected in the ssDNA of AAV.33 supplemented with increment doses of shRNA8 (ID=17) normalized to indels levels found in AAV.33 without ShRNA8 supplementation, as described in Example 18.

FIG. 71 is a bar graph displaying levels of editing as identified by FACS in mNPC-tdT cells infected with self-inactivating AAV vectors (AAV.32, AAV.33) or a non-self-inactivating viral vector (AAV.30), as described in Example 18. Black bars show editing from production with no shRNA supplemented to prevent self-cleavage during packaging, compared to white bars, showing editing levels from vectors supplemented with 3 increment doses of shRNA8 (ID=17) during production, as described in Example 18. Results from 2 viral MOI (3.0e+5, 1.0e+5) were displayed.

FIG. 72 is a schematic illustrating the various configurations for supplying an shRNA to reduce CasX expression during packaging, as described in Example 17. The shRNA, or shRNAs, can be supplied on the same plasmid as the AAV transgene, on another plasmid in production such as pRepCap or pHelper plasmid, on multiple production plasmids, or on a separate polynucleotide. The black boxes indicate ITRs.

FIG. 73 is a western blot showing silencing of CasX expression during AAV production using shRNA8, which was produced from constructs that contained the indicated shRNA scaffold (miR-Scribe, miR-E, miR-30a, or miR-Endo) and either a EF1α or U6 promoter, as described in Example 18. AAVs were produced from various constructs that contained different combinations of the indicated elements. Construct ID 30 was used as a base construct that did not contain any shRNA or STALL site (self-inactivating segments, also referred to herein as self-targeting alternative linked loci, or “STALL sites”).

FIG. 74A is a bar plot showing the western blot quantification of fold knockdown of CasX protein expression for each ‘no STALL’ experimental condition (indicated by construct ID) normalized to the CasX levels determined as a result of using the base construct (construct ID 30), as described in Example 18.

FIG. 74B is a bar chart showing the western blot quantification of fold knockdown of CasX protein expression for each ‘ATC STALL’ experimental condition (indicated by construct ID) normalized to the CasX levels determined as a result of using the base construct (construct ID 30), as described in Example 18.

FIG. 75 is a bar graph showing the quantification of siAAV genomes that were determined to be intact (lack of indels detected) by NGS for the indicated experimental conditions, as described in Example 18.

FIG. 76A is a graph showing the quantification of cleavage rates of RNP of CasX variant 491 and guide 174 on NTC PAMs, as described in Example 19. Timepoints were taken over the course of 10 minutes and the fraction cleaved was graphed for each target and timepoint. For NTC PAMs, only the first two minutes of the time course are shown for clarity.

FIG. 76B is a graph showing the quantification of cleavage rates of RNP of CasX variant 491 and guide 174 on NTT PAMs, as described in Example 19. Timepoints were taken over the course of 10 minutes and the fraction cleaved was graphed for each target and timepoint.

FIG. 77A is a bar graph displaying transcript levels detected for CasX, gRNA scaffold 174, and tdTomato in mice brain tissue harvested at three weeks post-treatment with AAVs with (Dual ATC STALL, Dual CTC STALL, or Single ATC STALL) or without (no STALL) the self-inactivation system, as described in Example 21.

FIG. 77B is a bar graph displaying transcript levels detected for CasX, gRNA scaffold 174, and tdTomato in mice brain tissue harvested at eight weeks post-treatment with AAVs with (Dual ATC STALL, Dual CTC STALL, or Single ATC STALL) or without (no STALL) the self-inactivation system, as described in Example 21.

FIG. 78 is a bar chart showing the western blot quantification of CasX protein levels at the three-week time point in mouse brain tissue treated with AAV-no STALL, siAAV Dual ATC STALL, siAAV Dual CTC STALL, or siAAV Single ATC STALL, as described in Example 21. CasX protein levels for each siAAV condition was normalized relative to the CasX level in the No STALL group.

FIG. 79 is a bar graph illustrating the quantification of editing levels at the tdTomato locus identified by histology and cell counting in brain tissue harvested at the three-week time point from mice treated with the indicated conditions, as described in Example 21. NT indicates the group of mice treated with AAVs containing the non-targeting spacer.

FIG. 80 is a schematic of the general configuration of a construct that would encode for a decoy gRNA, supplied on the same plasmid as the AAV transgene, as an alternative strategy to reduce CasX-mediated editing of the AAV transgene during production, as explored in Example 22. The illustration shows a generic siAAV transgene plasmid, where a U6-driven transcriptional unit that would encode for a decoy gRNA was designed 5′ of the CasX nuclease construct with flanking STALL sites.

FIG. 81 is a bar chart showing the normalized ratio of CasX titer to bGH titer for each experimental condition testing the effects of using a decoy gRNA for rescuing siAAV titer in the producing cells, as described in Example 22. Here, the CasX nuclease construct in the AAV transgene was flanked by either side with a TTCN STALL site, and decoy gRNAs were designed with guide scaffolds 174, 234, and 235. S=scrambled sequence; NT=non-targeting spacer; NS=no spacer, scaffold only.

FIG. 82 is a graph showing the results of an editing assay using AAV transgene plasmids nucleofected into hNPCs, as described in Example 23, demonstrating that CpG reduction or depletion within the U1a promoter (construct ID 178 and 179), U6 promoter (construct ID 180 and 181), or bGH poly(A) (construct ID 182) did not significantly reduce CasX-mediated editing at the B2M locus compared to the editing achieved with the original CpG+ AAV vector (construct ID 177). The controls used in this experiment were the non-targeting (NT) spacer and no treatment (NTx).

FIG. 83 is a bar plot depicting the results of an editing assay measured as indel rate detected by NGS at the human B2M locus in human induced neurons (iNs) seven days post-transduction with AAVs expressing CasX 491 driven by the various protein promoters as indicated at an MOI of 1E3 or 3E3, as described in Example 23.

FIG. 84A is a bar plot that illustrate the quantification of percent editing at the B2M locus as detected by NGS seven days post-transduction of AAVs into human iNs at an MOI of 3E3, as described in Example 23.

FIG. 84B is a bar plot that illustrate the quantification of percent editing at the B2M locus as detected by NGS seven days post-transduction of AAVs into human iNs at an MOI of 1E3, as described in Example 23.

FIG. 85 is a general schematic depicting various configurations in which the shRNA transcriptional unit can be arranged and stacked when supplied on the same plasmid as the AAV transgene, as described in Example 26. The black boxes indicate ITRs.

FIG. 86 is a bar chart showing the quantification of fold knockdown of CasX protein expression for each construct tested (indicated by construct ID) relative to the CasX levels determined as a result of using construct ID 32, as described in Example 26. Key attributes of each construct are shown in the table below the bar chart.

FIG. 87 is a bar graph depicting the quantification of siAAV genomes that were determined to be intact (lack of indels detected) by NGS for the indicated constructs, as described in Example 26. Key attributes of each construct are shown in the table below the bar graph.

FIG. 88 is a bar chart showing the quantification of fold knockdown of CasX protein expression for each construct tested (indicated by construct ID) relative to the CasX levels determined as a result of using construct ID 139, as described in Example 26. Key attributes of each construct are shown in the table below the bar chart.

FIG. 89 is a bar graph depicting the quantification of ssAAV genomes that were determined to be intact (lack of indels detected) by NGS for the indicated constructs, as described in Example 26. Key attributes of each construct are shown in the table below the bar graph.

FIG. 90 is a bar graph depicting the quantification of ssAAV genomes that were determined to be intact (lack of indels detected) by NGS for the indicated RepCap constructs, as described in Example 27. Construct ID 146 was used for the packaged AAV transgene. Key attributes of each construct are shown in the table below the bar graph.

FIG. 91 is a graph plotting the RNA abundance ratio, determined as log 2(cDNA reads/viral DNA input reads) calculated across ten summed technical replicates per unique poly(A) library member assessed during the high-throughput screen, as described in Example 6. The depicted data were for one biological replicate. The bGH poly(A) sequence is highlighted as a positive control.

FIG. 92 is a schematic of the regions and domains of a guide RNA used to design a scaffold library, as described in Example 28.

FIG. 93 is a pie chart of the relative distribution and design of the scaffold library with both unbiased (double and single mutations) and targeted mutations (towards the triplex, scaffold stem bubble, pseudoknot, and extended stem and loop) indicated, as described in Example 28.

FIG. 94 is a schematic of the triplex mutagenesis designed to specifically incorporate alternate triplex-forming base pairs into the triplex, as described in Example 28. Solid lines indicate the Watson-Crick pair in the triplex; the third strand nucleotide is indicated as a dotted line representing the non-canonical interaction with the purine of the duplex. In the library, each of the 5 locations indicated was replaced with all possible triplex motifs (G:GC, T:AT, G:GC)=243 sequences. The sequence shown is:

(SEQ ID NO: 2332)
ACUGGCGCUUUUAUCUGAUUACUUUGAGAGCCAUCANNNAUCAAAG.

FIG. 95 is a bar chart with results of the enrichment values of reference guide scaffolds 174 and 175 in each screen, as described in Example 28.

FIG. 96 is a flow-chart illustrating the qualitative relationship between tested combinations of mutations and their effect on both activity and specificity of the resulting CasX variants, as described in Example 29.

FIG. 97 is a pair of heat maps for single mutants in guide scaffolds 174 (SEQ ID NO: 2238) and 175 (SEQ ID NO: 2239) showing specific mutable regions in the scaffold across the sequences, as described in Example 28. Yellow shades reflect values with similar enrichment to the reference scaffolds; red shades indicate an increase in enrichment, and thus activity, relative to the reference scaffold; blue shades indicate a loss of activity relative to the wildtype scaffold; white indicates missing data (or a substitution that would result in wildtype sequence.

FIG. 98 is a scatterplot that compares the log 2 enrichment of single nucleotide mutations on reference guide scaffolds 174 and 175, as described in Example 28. Only those mutations to positions that were analogous between 174 and 175 are shown. Results suggest that, overall, guide scaffold 174 is more tolerant to changes than 175.

FIG. 99 is a bar chart showing the average (and 95% confidence interval) log 2 enrichment values for a set of scaffolds in which the pseudoknot pairs have been shuffled, such that each new pseudoknot has the same composition of base pairs, but in a different order within the stem, as described in Example 28. Each bar represents a set of scaffolds with the G:A (or A:G) pair location indicated (see diagram at right). 291 pseudoknot stems were tested; numbers above bars indicate the number of stems with the G:A (or A:G) pair at each position.

FIG. 100 is a schematic of the pseudoknot sequence of FIG. 92, given 5′ to 3′, with the two strand sequences separated by an underscore.

FIG. 101 is a bar chart showing the average (and 95% confidence interval) log 2 enrichment values for scaffolds, divided by the predicted secondary structure stability of the pseudoknot stem region, as described in Example 28. Scaffolds with very stable stems (e.g., ΔG<−7 kcal/mol) had high enrichment values on average, whereas scaffolds with destabilized stems (ΔG≥−5 kcal/mol) had low enrichment values on average.

FIG. 102 is a heat map of all double mutants of positions 7 and 29 in scaffold 175, as described in Example 28. The pseudoknot sequence is given 5′ to 3′, on the right.

FIG. 103 is a graph of a survival assay to determine the selective stringency of the CcdB selection to different spacers when targeted by CasX protein 515 and scaffold 174, as described in Example 29.

FIG. 104 illustrates the schematics of AAV plasmid constructs containing various configurations of the gRNA transcriptional unit (Pol III U6 promoter driving the expression of the gRNA scaffold and indicated spacer) as described in Example 7.

FIG. 105 is a graph showing the quantification of percent editing at the tdTomato locus in mNPCs five days post-transduction with AAVs produced from the indicated AAV constructs, as described in Example 7. Editing was assessed by FACS five days post-transduction.

FIG. 106 depicts the results of an editing assay measured as indel rate detected by NGS at the DMPK 3′ UTR locus for the indicated AAV dual-guide systems transduced into HEK293T cells in a series of three-fold dilution, as described in Example 9.

FIG. 107 is a bar chart displaying the breakdown of indels generated by type of editing (single edit at the 5′ or 3′ of CTG repeat or double-cut resulting in CTG repeat dropout) at the DMPK 3′ UTR locus by AAVs harboring the dual guides and 20.7 and 20.11 spacer combination, as described in Example 9. The percentage of single or dual-edits were calculated from the total percent of reads analyzed.

FIG. 108 is a bar plot showing the quantification of percent editing measured as indel rate detected by NGS at the ROSA26 locus for the indicated AAV constructs nucleofected into C2C12 myoblasts or mouse NPCs to assess the effects of individual muscle-specific promoters on editing rates, as described in Example 30.

FIG. 109 is a scatter plot of percent editing versus promoter size for all the AAV constructs with varying promoters tested, as described in Example 30.

FIG. 110A is a diagram of the secondary structure of guide RNA scaffold 235, noting the regions with CpG motifs, as described in Example 23. CpG motifs in (1) the pseudoknot stem, (2) the scaffold stem, (3) the extended stem bubble, (4) the extended step, and (5) the extended stem loop are labeled on the structure.

FIG. 110B is a diagram of the CpG-reducing mutations that were introduced into each of the five regions in the coding sequence of the guide RNA scaffold, as described in Example 23.

FIG. 111A provides the results of an editing experiment in which AAV vectors with various CpG-reduced or CpG-depleted guide RNA scaffolds were used to edit the B2M locus in induced neurons, as described in Example 23. The AAV vectors were administered at a multiplicity of infection (MOI) of 4e3. The bars show the mean±the SD of two replicates per sample. “No Tx” indicates a non-transduced control, and “NT” indicates a control with a non-targeting spacer.

FIG. 111B provides the results of an editing experiment in which AAV vectors with various CpG-reduced or CpG-depleted guide RNA scaffolds were used to edit the B2M locus in induced neurons, as described in Example 23. The AAV vectors were administered at an MOI of 3e3. The bars show the mean±the SD of two replicates per sample. “No Tx” indicates a non-transduced control.

FIG. 111C provides the results of an editing experiment in which AAV vectors with various CpG-reduced or CpG-depleted guide RNA scaffolds were used to edit the B2M locus in induced neurons, as described in Example 23. The AAV vectors were administered at an MOI of 1e3. The bars show the mean±the SD of two replicates per sample. “No Tx” indicates a non-transduced control.

FIG. 111D provides the results of an editing experiment in which AAV vectors with various CpG-reduced or CpG-depleted guide RNA scaffolds were used to edit the B2M locus in induced neurons, as described in Example 23. The AAV vectors were administered at an MOI of MOI=3e2. The bars show the mean±the SD of two replicates per sample. “No Tx” indicates a non-transduced control.

FIG. 112 shows the schematics of AAV constructs with additional alternative gRNA configurations for constructs having two gRNAs, as described in Example 9. The tapered points depict the orientation of the transcriptional unit for CasX protein or gRNA.

FIG. 113 is a bar graph depicting the quantification of siAAV genomes that were determined to be intact (lack of indels detected) by NGS for the indicated constructs, which contained STALL sites with alternative PAMs, as described in Example 12.

FIG. 114 is a bar plot showing the quantification of ssAAV genomes that were determined to be intact (lack of indels detected) by NGS for the AAVs containing the indicated STALL sites produced using pRepCap construct 167, as described in Example 27.

FIG. 115A is a bar graph showing the quantification of percent editing measured as indel rate detected at the ROSA26 locus in C2C12 myoblasts and myotubes transduced with AAVs containing the indicated promoters to drive CasX expression at an MOI of 3E5 vg/cell, as described in Example 30.

FIG. 115B is a bar graph showing the quantification of percent editing measured as indel rate detected at the ROSA26 locus in C2C12 myoblasts and myotubes transduced with AAVs containing the indicated promoters to drive CasX expression at an MOI of 1E5 vg/cell, as described in Example 30.

FIG. 116 is a bar graph showing the quantification of percent editing measured as indel rate detected at the ROSA26 locus in the indicated tissues harvested from mice injected with AAVs containing the indicated promoters driving CasX expression, as described in Example 30. As experimental controls, mice were either untreated (naïve) or injected with AAVs containing UbC promoter driving CasX expression with a non-targeting gRNA. N=3 animals per promoter experimental condition; N=2 animals for the untreated control group.

FIG. 117 is a bar graph quantifying average CasX expression, normalized by vg/dg, driven by muscle-specific promoters CK8e or MHC7 relative to CasX expression driven by UbC, for the indicated tissues harvested from mice injected with AAVs containing the indicated promoters, as described in Example 30. N=3 animals per promoter experimental condition.

FIG. 118 is a box plot showing the quantification of percent editing at the ROSA26 locus in retinae harvested from mice treated with subretinal injections of AAVs expressing CasX 491 driven by the indicated photoreceptor-specific promoters with a ROSA26-targeting spacer, as described in Example 34. The dashed line indicates the theoretical maximum editing of photoreceptors that can be achieved with optimal transduction.

FIG. 119A is a panel of scatterplots for promoter variants GRK1(292)-SV40 and GRK1(292), showing the correlation of vg/dg with the editing level achieved for a particular promoter used to drive CasX expression in the retinae, as described in Example 34. A nonlinear regression curve was fitted to assess the correlation, and the values of the slopes, along with their corresponding standard deviation values, of these curves were determined and reported in Table 59.

FIG. 119B is a panel of scatterplots for promoter variants GRK1(241) and GRK1(199), showing the correlation of vg/dg with the editing level achieved for a particular promoter used to drive CasX expression in the retinae, as described in Example 34. A nonlinear regression curve was fitted to assess the correlation, and the values of the slopes, along with their corresponding standard deviation values, of these curves were determined and reported in Table 59.

FIG. 119C is a panel of scatterplots for the indicated promoter variants GRK1(94) and GRK1(93), showing the correlation of vg/dg with the editing level achieved for a particular promoter used to drive CasX expression in the retinae, as described in Example 34. A nonlinear regression curve was fitted to assess the correlation, and the values of the slopes, along with their corresponding standard deviation values, of these curves were determined and reported in Table 59.

FIG. 120 is a bar plot showing the results of an editing assay at the tdTomato locus assessed by FACS in mNPCs nucleofected with AAV plasmids encoding for AAVs expressing the CasX:dual-gRNA system with the indicated configurations and spacer combinations for the two gRNA units relative to the CasX construct, as described in Example 37. The “R” preceding the spacer denotes the reverse orientation of the transcription of the indicated gRNA unit. An AAV plasmid encoding for AAVs expressing CasX 491 with a single gRNA transcriptional unit using spacer 12.7 as well as an untreated well served as experimental controls.

FIG. 121A is a line graph showing the results of an editing assay at the tdTomato locus assessed by FACS in mNPCs transduced with AAVs expressing the CasX:dual-gRNA system at varying MOIs, with the indicated spacer combinations of the two gRNA units arranged in configuration #1 relative to the CasX construct, as described in Example 37. An untreated control was included for comparison.

FIG. 121B is a line graph showing the results of an editing assay at the tdTomato locus assessed by FACS in mNPCs transduced with AAVs expressing the CasX:dual-gRNA system at varying MOIs, with the indicated spacer combinations of the two gRNA units arranged in configuration #4 relative to the CasX construct, as described in Example 37. The “R” preceding the spacer denotes the reverse orientation of the transcription of the indicated gRNA unit. An untreated control was included for comparison.

FIG. 121C is a line graph showing the results of an editing assay at the tdTomato locus assessed by FACS in mNPCs transduced with AAVs expressing the CasX:dual-gRNA system at varying MOIs, with the indicated spacer combinations of the two gRNA units arranged in configuration #2 relative to the CasX construct, as described in Example 37. An untreated control was included for comparison.

FIG. 122 is a bar graph showing the results of an editing assay at the tdTomato locus assessed by FACS in mNPCs transduced with AAVs expressing the CasX:dual-gRNA system for indicated configurations #1, #4, and #2, as described in Example 37. AAVs expressing CasX 491 with a single gRNA transcriptional unit using spacer 12.7 served as an experimental control.

FIG. 123A is a box plot showing median, minimal and highest editing values using AAV-mediated expression of CasX 491 detected by NGS 3 weeks post-injection in wild-type retinae injected with 5.0e+9 vg/eye of AAV.X.491.174.11.30 vectors, in which the 491 protein is driven by promoter variants designed to selectively express in rod photoreceptors (X=RP1-RP5) or a ubiquitous promoter (X=CMV), as described in Example 33. The grey line is placed at the editing levels achieved by AAV.RP1.491.174.11.30 to compare to other viral vectors tested.

FIG. 123B is a plot displaying levels of editing achieved by AAV vectors in wild-type retinae injected with 5.0e+9 vg/eye of AAV.X.491.174.11.30 vectors, compared to total transgene size (bp), as described in Example 33. The grey line delimitates transgenes below or above 4.9 kb size.

FIG. 124 is a graph showing in vivo editing results that AAV-mediated expression of CasX 491 and gRNA spacer 174.4.76 in rod photoreceptors led to detectable levels of editing levels at integrated Nrl-GFP locus in a dose-dependent manner, as described in Example 33. The bar graph shows editing levels detected by NGS at the integrated GFP locus 4-weeks and 12-weeks post-injection in heterozygous Nrl-GFP mice injected with the indicated doses of AAV.RP1.491.174.4.76 vectors in one eye, and the vehicle control in the contralateral eye).

FIG. 125A is a scatter boxplot representing levels of GFP protein detected in the western blots and quantified by densitometry, from which ratios of densitometric values of the GFP band for total amount of proteins were normalized to the vehicle group levels, as described in Example 33. One-way ANOVA statistical analysis was performed (*=p<0.5).

FIG. 125B is a plot correlating GFP protein fraction to levels of percent editing achieved in mouse retinae of the AAV-treated mice, for both the 1.0e+9 and 1.0e+10 dose groups, as described in Example 33.

FIG. 126A is a bar graph representing the ratio of GFP fluorescence levels (superior to inferior retina mean grey values) detected by fundus imaging at 4-weeks compared to 12-weeks post-injection in mice injected with two dose levels of AAV constructs, as described in Example 33.

FIG. 126B displays representative images of fluorescence fundus imaging of GFP in retina from mice injected with 1.0e+9 vg (#13) or 1.0e+10 vg (#34) with the AAV constructs at 4-weeks and (left panel) or 12-weeks (right panel), as described in Example 33.

FIG. 127 present histology images or retinae of mice stained with various immunochemistry reagents, as described in Example 33, confirming efficient knock-down of GFP in photoreceptor cells in an AAV-dose dependent manner. The images are representative confocal images of cross-sectioned retinae injected with vehicle (panels A, B, C, D), AAV-CasX at a 1.0e+9 vg dose (panels E, F, G, and H) and 1.0E+10 vg dose (panels I, J, K, and L). Structural imaging shows GFP expression by rod photoreceptors in the outer segment (images in panels A, E, I and images in panels C, G, and K for 20× and 40× magnifications, respectively). Cell nuclei were counterstained with Hoechst (panels B, F, and J) and cells stained with anti-HA to correlate levels of HA (CasX transgene levels; panels D, H, and L; 40× magnification) and GFP expressed in photoreceptors. White box outlines in B and F indicate retinal regions analyzed at 40× magnification in panels C and G. Legend: RPE=retinal pigment epithelium, OS=outer segment, ONL=outer nuclear layer, INL=inner nuclear layer, GCL=ganglion.

FIG. 128 is a bar graph showing the number of viral genomes per diploid genome (vg/dg) in the right hemisphere of the cortex of mice administered siAAVs with zero, one, or two STALL sites three weeks following administration, as described in Example 35.

FIG. 129A is a bar graph showing the abundance of mRNA encoding CasX in the right hemisphere of the cortex of mice administered siAAVs with zero, one, or two STALL sites three weeks following administration, as described in Example 35.

FIG. 129B is a bar graph showing the abundance of guide scaffold 235 RNA in the right hemisphere of the cortex of mice administered siAAVs with zero, one, or two STALL sites three weeks following administration, as described in Example 35.

FIG. 130 is a bar graph showing a quantification of percent editing measured as indel rate detected at the ROSA26 locus in mice administered siAAVs with zero, one, or two STALL sites three weeks following administration, as described in Example 35. Editing was measured in the right hemisphere of the cortex.

FIG. 131 is a bar graph showing the number of viral genomes per diploid genome (vg/dg) in the liver and cortex of mice administered siAAVs with zero, one, or two STALL sites 16 weeks following administration, as described in Example 35. The vg/dg in the cortex is the average of the right and left hemispheres of the cortex for each animal.

FIG. 132A is a bar graph showing the abundance of mRNA encoding CasX in the liver of mice administered siAAVs with zero, one, or two STALL sites three weeks following administration, as described in Example 35.

FIG. 132B is a bar graph showing the abundance of mRNA encoding CasX in the cortex of mice administered siAAVs with zero, one, or two STALL sites three weeks following administration, as described in Example 35. The mRNA abundance in the cortex is the average of the right and left hemispheres of the cortex for each animal.

FIG. 133A is a bar graph showing the abundance of guide scaffold 235 RNA in the liver of mice administered siAAVs with zero, one, or two STALL sites three weeks following administration, as described in Example 35.

FIG. 133B is a bar graph showing the abundance of guide scaffold 235 RNA in the cortex of mice administered siAAVs with zero, one, or two STALL sites three weeks following administration, as described in Example 35. The RNA abundance in the cortex is the average of the right and left hemispheres of the cortex for each animal.

FIG. 134 is a bar graph showing a quantification of percent editing measured as indel rate detected at the ROSA26 locus in mice administered siAAVs with zero, one, or two STALL sites 16 weeks following administration, as described in Example 35. Editing was measured in the right hemisphere of the cortex, the left hemisphere of the cortex, and the liver, as indicated on the y-axis.

FIG. 135A is a bar plot showing the quantification of percent editing at the B2M locus in human induced neurons (iNs) transduced with AAVs expressing the indicated constructs containing various poly(A) signal sequences at an MOI of 1E2 vg/cell, as described in Example 6.

FIG. 135B is a bar plot showing the quantification of percent editing at the B2M locus in human induced neurons (iNs) transduced with AAVs expressing the indicated constructs containing various poly(A) signal sequences at an MOI of 1E3 vg/cell, as described in Example 6.

### DETAILED DESCRIPTION

While exemplary embodiments have been shown and described herein, it will be obvious to those skilled in the art that such embodiments are provided by way of example only. Numerous variations, changes, and substitutions will now occur to those skilled in the art without departing from the inventions claimed herein. It should be understood that various alternatives to the embodiments described herein may be employed in practicing the embodiments of the disclosure. It is intended that the claims define the scope of the invention and that methods and structures within the scope of these claims and their equivalents be covered thereby.

Unless otherwise defined, all technical and scientific terms used herein have the same meaning as commonly understood by one of ordinary skill in the art to which this invention belongs. Although methods and materials similar or equivalent to those described herein can be used in the practice or testing of the present embodiments, suitable methods and materials are described below. In case of conflict, the patent specification, including definitions, will control. In addition, the materials, methods, and examples are illustrative only and not intended to be limiting. Numerous variations, changes, and substitutions will now occur to those skilled in the art without departing from the invention.

### Definitions

“Hybridizable” or “complementary” are used interchangeably to mean that a nucleic acid (e.g., RNA, DNA) comprises a sequence of nucleotides that enables it to non-covalently bind, i.e., form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. It is understood that the sequence of a polynucleotide need not be 100% complementary to that of its target nucleic acid to be specifically hybridizable; it can have at least about 70%, at least about 80%, or at least about 90%, or at least about 95% sequence identity and still hybridize to the target nucleic acid. Moreover, a polynucleotide may hybridize over one or more segments such that intervening or adjacent segments are not involved in the hybridization event (e.g., a loop structure or hairpin structure, a ‘bulge’, ‘bubble’ and the like). Thus, the skilled artisan will understand that while individual bases within a sequence may not be complementary to another sequence, the sequence as a whole is still considered to be complementary.

A “gene,” for the purposes of the present disclosure, includes a DNA region encoding a gene product (e.g., a protein, RNA), as well as all DNA regions which regulate the production of the gene product, whether or not such regulatory sequences are adjacent to coding and/or transcribed sequences. Accordingly, a gene may include accessory element sequences including, but not necessarily limited to, promoter sequences, terminators, translational regulatory sequences such as ribosome binding sites and internal ribosome entry sites, enhancers, silencers, insulators, boundary elements, replication origins, matrix attachment sites and locus control regions. Coding sequences encode a gene product upon transcription or transcription and translation; the coding sequences of the disclosure may comprise fragments and need not contain a full-length open reading frame. A gene can include both the strand that is transcribed as well as the complementary strand containing the anticodons.

As used herein, a singular reference to an agent may also refer to a plurality of the agent, e.g., vector and vectors. Similarly, the terms “polynucleotide,” “polynucleotide sequence,” “nucleotide,” “nucleic acid” and “nucleic acid sequence” may be used interchangeably.

The term “downstream” refers to a nucleotide sequence that is located 3′ to a reference nucleotide sequence. In certain embodiments, downstream nucleotide sequences relate to sequences that follow the starting point of transcription. For example, the translation initiation codon of a gene is located downstream of the start site of transcription.

The term “upstream” refers to a nucleotide sequence that is located 5′ to a reference nucleotide sequence. In certain embodiments, upstream nucleotide sequences relate to sequences that are located on the 5′ side of a coding region or starting point of transcription. For example, most promoters are located upstream of the start site of transcription.

The term “adjacent to” with respect to polynucleotide or amino acid sequences refers to sequences that are next to, or adjoining each other in a polynucleotide or polypeptide. The skilled artisan will appreciate that two sequences can be considered to be adjacent to each other and still encompass a limited amount of intervening sequence, e.g., 1, 2, 3, 4, 5, 6, 7, 8, 9 or 10 nucleotides or amino acids.

The term “regulatory element” is used interchangeably herein with the term “regulatory sequence,” and is intended to include promoters, enhancers, and other expression regulatory elements. It will be understood that the choice of the appropriate regulatory element will depend on the encoded component to be expressed (e.g., protein or RNA) or whether the nucleic acid comprises multiple components that require different polymerases or are not intended to be expressed as a fusion protein.

The term “accessory element” is used interchangeably herein with the term “accessory sequence,” and is intended to include, inter alia, polyadenylation signals (poly(A) signal), enhancer elements, introns, posttranscriptional regulatory elements (PTREs), nuclear localization signals (NLS), deaminases, DNA glycosylase inhibitors, additional promoters, factors that stimulate CRISPR-mediated homology-directed repair (e.g. in cis or in trans), activators or repressors of transcription, self-cleaving sequences, and fusion domains, for example a fusion domain fused to a CRISPR protein. It will be understood that the choice of the appropriate accessory element or elements will depend on the encoded component to be expressed (e.g., protein or RNA) or whether the nucleic acid comprises multiple components that require different polymerases or are not intended to be expressed as a fusion protein.

The term “promoter” refers to a DNA sequence that contains a transcription start site and additional sequences to facilitate polymerase binding and transcription. Exemplary eukaryotic promoters include elements such as a TATA box, and/or B recognition element (BRE) and assists or promotes the transcription and expression of an associated transcribable polynucleotide sequence and/or gene (or transgene). A promoter can be synthetically produced or can be derived from a known or naturally occurring promoter sequence or another promoter sequence. A promoter can be proximal or distal to the gene to be transcribed. A promoter can also include a chimeric promoter comprising a combination of two or more heterologous sequences to confer certain properties. A promoter of the present disclosure can include variants of promoter sequences that are similar in composition, but not identical to, other promoter sequence(s) known or provided herein. A promoter can be classified according to criteria relating to the pattern of expression of an associated coding or transcribable sequence or gene operably linked to the promoter, such as constitutive, developmental, tissue-specific, inducible, etc. A promoter can also be classified according to its strength. As used in the context of a promoter, “strength” refers to the rate of transcription of the gene controlled by the promoter. A “strong” promoter means the rate of transcription is high, while a “weak” promoter means the rate of transcription is relatively low.

A promoter of the disclosure can be a Polymerase II (Pol II) promoter. Polymerase II transcribes all protein coding and many non-coding genes. A representative Pol II promoter includes a core promoter, which is a sequence of about 100 base pairs surrounding the transcription start site, and serves as a binding platform for the Pol II polymerase and associated general transcription factors. The promoter may contain one or more core promoter elements such as the TATA box, BRE, Initiator (INR), motif ten element (MTE), downstream core promoter element (DPE), downstream core element (DCE), although core promoters lacking these elements are known in the art.

A promoter of the disclosure can be a Polymerase III (Pol III) promoter. Pol III transcribes DNA to synthesize small ribosomal RNAs such as the 5S rRNA, tRNAs, and other small RNAs. Representative Pol III promoters use internal control sequences (sequences within the transcribed section of the gene) to support transcription, although upstream elements such as the TATA box are also sometimes used. All Pol III promoters are envisaged as within the scope of the instant disclosure.

The term “enhancer” refers to regulatory DNA sequences that, when bound by specific proteins called transcription factors, regulate the expression of an associated gene. Enhancers may be located in the intron of the gene, or 5′ or 3′ of the coding sequence of the gene. Enhancers may be proximal to the gene (i.e., within a few tens or hundreds of base pairs (bp) of the promoter), or may be located distal to the gene (i.e., thousands of bp, hundreds of thousands of bp, or even millions of bp away from the promoter). A single gene may be regulated by more than one enhancer, all of which are envisaged as within the scope of the instant disclosure.

As used herein, a “post-transcriptional regulatory element (PTRE),” such as a hepatitis PTRE, refers to a DNA sequence that, when transcribed creates a tertiary structure capable of exhibiting post-transcriptional activity to enhance or promote expression of an associated gene operably linked thereto.

“Recombinant,” as used herein, means that a particular nucleic acid (DNA or RNA) is the product of various combinations of cloning, restriction, and/or ligation steps resulting in a construct having a structural coding or non-coding sequence distinguishable from endogenous nucleic acids found in natural systems. Generally, DNA sequences encoding the structural coding sequence can be assembled from cDNA fragments and short oligonucleotide linkers, or from a series of synthetic oligonucleotides, to provide a synthetic nucleic acid which is capable of being expressed from a recombinant transcriptional unit contained in a cell or in a cell-free transcription and translation system. Such sequences can be provided in the form of an open reading frame uninterrupted by internal non-translated sequences, or introns, which are typically present in eukaryotic genes. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms (see “enhancers” and “promoters”, above).

The term “recombinant polynucleotide” or “recombinant nucleic acid” refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a redundant codon encoding the same or a conservative amino acid, while typically introducing or removing a sequence recognition site. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques.

Similarly, the term “recombinant polypeptide” or “recombinant protein” refers to a polypeptide or protein which is not naturally occurring, e.g., is made by the artificial combination of two otherwise separated segments of amino sequence through human intervention. Thus, e.g., a protein that comprises a heterologous amino acid sequence is recombinant.

As used herein, “lipid nanoparticle” refers to a transfer vehicle comprising one or more lipids (e.g., cationic lipids, non-cationic lipids, and PEG-modified lipids). Preferably, the lipid nanoparticles are formulated to contain and to deliver one or more vectors to one or more target cells. Examples of suitable lipids include, for example, the phosphatidyl compounds (e.g., phosphatidylglycerol, phosphatidylcholine, phosphatidylserine, phosphatidylethanolamine, sphingolipids, cerebrosides, and gangliosides).

As used herein, the term “contacting” means establishing a physical connection between two or more entities. For example, contacting a target nucleic acid with a guide nucleic acid means that the target nucleic acid and the guide nucleic acid are made to share a physical connection; e.g., can hybridize if the sequences share sequence similarity.

As used herein, the term “self-inactivating recombinant vector” or “SIRV” are compositions wherein the expression or activity of one or more components encoded by the polynucleotide of the self-inactivating recombinant vector is capable of being diminished or eliminated by cleavage of a polynucleotide by an RNP of the CRISPR nuclease and a guide RNA encoded by the polynucleotide, resulting in the inability of one or more of the CRISPR components of the vector to be subsequently expressed. For clarity, self-inactivating should not be taken to mean that all such constructs are rendered inactive with respect to the expression and function of the CRISPR nuclease and guide RNA, but that the expression or activity of the components may be reduced.

“Dissociation constant”, or “K_{d}”, are used interchangeably and mean the affinity between a ligand “L” and a protein “P”; i.e., how tightly a ligand binds to a particular protein. It can be calculated using the formula K_{d}=[L]/[P]/[LP], where [P], [L] and [LP] represent molar concentrations of the protein, ligand and complex, respectively.

The disclosure provides compositions and methods useful for modifying a target nucleic acid. As used herein “modifying” and “modification” are used interchangeably and include, but are not limited to, cleaving, nicking, editing, deleting, knocking in, knocking out, and the like.

The term “knock-out” refers to the elimination of a gene or the expression of a gene. For example, a gene can be knocked out by either a deletion or an addition of a nucleotide sequence that leads to a disruption of the reading frame. As another example, a gene may be knocked out by replacing a part of the gene with an irrelevant sequence. The term “knock-down” as used herein refers to reduction in the expression of a gene or its gene product(s). As a result of a gene knock-down, the protein activity or function may be attenuated or the protein levels may be reduced or eliminated.

As used herein, “homology-directed repair” (HDR) refers to the form of DNA repair that takes place during repair of double-strand breaks in cells. This process requires nucleotide sequence homology, and uses a donor template to repair or knock-out a target DNA, and leads to the transfer of genetic information from the donor to the target. Homology-directed repair can result in an alteration of the sequence of the target sequence by insertion, deletion, or mutation if the donor template differs from the target DNA sequence and part or all of the sequence of the donor template is incorporated into the target DNA.

As used herein, “non-homologous end joining” (NHEJ) refers to the repair of double-strand breaks in DNA by direct ligation of the break ends to one another without the need for a homologous template (in contrast to homology-directed repair, which requires a homologous sequence to guide repair). NHEJ often results in the loss (deletion) of nucleotide sequence near the site of the double-strand break.

As used herein “micro-homology mediated end joining” (MMEJ) refers to a mutagenic DSB repair mechanism, which always associates with deletions flanking the break sites without the need for a homologous template (in contrast to homology-directed repair, which requires a homologous sequence to guide repair). MMEJ often results in the loss (deletion) of nucleotide sequence near the site of the double-strand break.

A polynucleotide or polypeptide has a certain percent “sequence similarity” or “sequence identity” to another polynucleotide or polypeptide, meaning that, when aligned, that percentage of bases or amino acids are the same, and in the same relative position, when comparing the two sequences. Sequence similarity (sometimes referred to as percent similarity, percent identity, or homology) can be determined in a number of different manners. To determine sequence similarity, sequences can be aligned using the methods and computer programs that are known in the art, including BLAST, available over the world wide web at ncbi.nlm.nih.gov/BLAST. Percent complementarity between particular stretches of nucleic acid sequences within nucleic acids can be determined using any convenient method. Example methods include BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), e.g., using default settings, which uses the algorithm of Smith and Waterman (Adv. Appl. Math., 1981, 2, 482-489).

The terms “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence.

A “vector” or “expression vector” is a replicon, such as plasmid, phage, virus, or cosmid, to which another DNA segment, i.e., an expression cassette, may be attached so as to bring about the replication or expression of the attached segment in a cell.

The term “naturally-occurring” or “unmodified” or “wild type” as used herein as applied to a nucleic acid, a polypeptide, a cell, or an organism, refers to a nucleic acid, polypeptide, cell, or organism that is found in nature.

As used herein, a “mutation” refers to an insertion, deletion, substitution, duplication, or inversion of one or more amino acids or nucleotides as compared to a wild-type or reference amino acid sequence or to a wild-type or reference nucleotide sequence.

As used herein the term “isolated” is meant to describe a polynucleotide, a polypeptide, or a cell that is in an environment different from that in which the polynucleotide, the polypeptide, or the cell naturally occurs. An isolated genetically modified host cell may be present in a mixed population of genetically modified host cells.

A “host cell,” as used herein, denotes a eukaryotic cell, a prokaryotic cell, or a cell from a multicellular organism (e.g., a cell line) cultured as a unicellular entity, which eukaryotic or prokaryotic cells are used as recipients for a nucleic acid (e.g., an AAV vector), and include the progeny of the original cell which has been genetically modified by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an AAV vector.

A “target cell marker” refers to a molecule expressed by a target cell including but not limited to cell-surface receptors, cytokine receptors, antigens, tumor-associated antigens, glycoproteins, oligonucleotides, enzymatic substrates, antigenic determinants, or binding sites that may be present in the on the surface of a target tissue or cell that may serve as ligands for an antibody fragment.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide-containing side chains consists of asparagine and glutamine; a group of amino acids having aromatic side chains consists of phenylalanine, tyrosine, and tryptophan; a group of amino acids having basic side chains consists of lysine, arginine, and histidine; and a group of amino acids having sulfur-containing side chains consists of cysteine and methionine. Exemplary conservative amino acid substitution groups are: valine-leucine-isoleucine, phenylalanine-tyrosine, lysine-arginine, alanine-valine, and asparagine-glutamine.

As used herein, “treatment” or “treating,” are used interchangeably herein and refer to an approach for obtaining beneficial or desired results, including but not limited to a therapeutic benefit and/or a prophylactic benefit. By therapeutic benefit is meant eradication or amelioration of the underlying disorder or disease being treated. A therapeutic benefit can also be achieved with the eradication or amelioration of one or more of the symptoms or an improvement in one or more clinical parameters associated with the underlying disease such that an improvement is observed in the subject, notwithstanding that the subject may still be afflicted with the underlying disorder.

The terms “therapeutically effective amount” and “therapeutically effective dose”, as used herein, refer to an amount of a drug or a biologic, alone or as a part of a composition, that is capable of having any detectable, beneficial effect on any symptom, aspect, measured parameter or characteristics of a disease state or condition when administered in one or repeated doses to a subject such as a human or an experimental animal. Such effect need not be absolute to be beneficial.

As used herein, “administering” means a method of giving a dosage of a compound (e.g., a composition of the disclosure) or a composition (e.g., a pharmaceutical composition) to a subject.

A “subject” is a mammal. Mammals include, but are not limited to, domesticated animals, non-human primates, humans, dogs, rabbits, mice, rats and other rodents.

All publications, patents, and patent applications mentioned in this specification are herein incorporated by reference to the same extent as if each individual publication, patent, or patent application was specifically and individually indicated to be incorporated by reference.

### I. General Methods

The practice of the present invention employs, unless otherwise indicated, conventional techniques of immunology, biochemistry, chemistry, molecular biology, microbiology, cell biology, genomics and recombinant DNA, which can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

Where a range of values is provided, it is understood that endpoints are included and that each intervening value, to the tenth of the unit of the lower limit unless the context clearly dictates otherwise, between the upper and lower limit of that range and any other stated or intervening value in that stated range, is encompassed. The upper and lower limits of these smaller ranges may independently be included in the smaller ranges, and are also encompassed, subject to any specifically excluded limit in the stated range. Where the stated range includes one or both of the limits, ranges excluding either or both of those included limits are also included.

Unless defined otherwise, all technical and scientific terms used herein have the same meaning as commonly understood by one of ordinary skill in the art to which this invention belongs. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise.

It will be appreciated that certain features of the disclosure, which are, for clarity, described in the context of separate embodiments, may also be provided in combination in a single embodiment. In other cases, various features of the disclosure, which are, for brevity, described in the context of a single embodiment, may also be provided separately or in any suitable sub-combination. It is intended that all combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### II. Self-Inactivating Recombinant Vectors

The present disclosure provides self-inactivating recombinant vectors (SIRV) designed to express Class 2 Type V CRISPR nucleases having a single RNA-guided RuvC domain and one or more guide RNAs to target cells and/or tissues for genetic editing or modification of a target nucleic acid. These SIRV temporally control the expression of one or more of the CRISPR components relative to the editing or modification of the target nucleic acid. A number of approaches to effect the self-inactivating feature of the constructs have been created by the differential design of features of the SIRV constructs of the disclosure and combinations thereof. The self-inactivating features of the SIRV are described herein, which, when incorporated into a viral vector or lipid nanoparticle, confer enhanced safety and a higher degree of specificity to the compositions when utilized for gene editing in a subject compared to systems not employing the self-inactivating features.

In some embodiments, the SIRV comprise a polynucleotide comprising components that include a packaging component, sequences encoding Class 2 Type V CRISPR components (e.g., nucleases and one or two guide RNA (gRNA)) under the control of regulatory elements and, optionally, one or more accessory elements, and one or more self-inactivating segments, also referred to herein as self-targeting alternative linked loci (“STALL”). The self-inactivating segment polynucleotides comprise a protospacer adjacent motif (PAM) sequence and a polynucleotide sequence capable of hybridizing with the targeting sequence of a gRNA (or is identical to the targeting sequence of a gRNA) encoded by the SIRV. In some embodiments, the self-inactivating segment polynucleotide comprises the 3 nucleotides of a PAM, an intervening nucleotide, and 15-21 nucleotides capable of hybridizing with the targeting sequence of a gRNA (or is identical to the targeting sequence of a gRNA given the double-stranded form of the episome). In a particular embodiment, the self-inactivating segment polynucleotide comprises the 3 nucleotides of a PAM, an intervening nucleotide, and 20 nucleotides capable of hybridizing with the targeting sequence of a gRNA (or is identical to the targeting sequence of a gRNA, given the double-stranded form of the episome). The double-stranded episomal form of the SIRV in a cell, which can include a transfected or transduced cell, is capable of being bound and cleaved by a ribonuclear protein complex (RNP) of the Class 2 CRISPR protein and a gRNA. In some embodiments, the SIRV are delivered directly to a target cell. In other embodiments, the SIRV are incorporated into virus particles, or lipid nanoparticles capable of delivering the SIRV to a target cell, described more fully, below. In either case, the inclusion of a packaging component (e.g., ITRs from AAV or lentivirus) can result in the formation of a double-stranded episomal form of the SIRV within the target cell to be modified.

The disclosure provides a number of SIRV design configurations that can be used singly or in combination; both for the purpose of providing the CRISPR editing components as well as self-inactivating features designed to reduce or eliminate the expression of the CRISPR components. It will be understood in the sections that follow that the SIRV can be used to transfect cells or can be incorporated into viral particles (such as AAV, adenovirus, lentivirus or gammaretrovirus) or lipid nanoparticles to transduce the cells. In exemplary embodiments, the SIRV is incorporated into an AAV. The description of these designs follow.

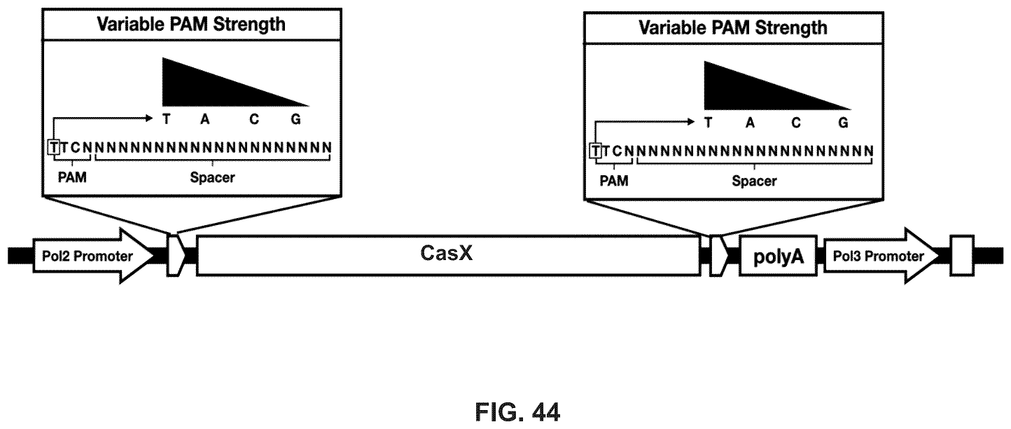

One or multiple copies (e.g., 1, 2, 3, 4 or more) of the self-inactivating segments can be present in several different regions of the SIRV constructs for each of the designs described herein. In some embodiments, an SIRV comprises a self-inactivating segment located: i) 5′ or 3′ adjacent to or within the sequence encoding the Class 2 CRISPR protein having a single RNA-guided RuvC domain; ii) 5′ or 3′ adjacent to or within a Kozak sequence located between the first promoter and the sequence encoding the Class 2 CRISPR protein; iii) 5′ or 3′ adjacent to or within to the first promoter sequence; iv) 5′ or 3′ adjacent to or within the second promoter sequence; v) downstream of the transcriptional start site for the sequence encoding the Class 2 CRISPR protein; vi) within one or more inserted introns in the polynucleotide encoding the Class 2 CRISPR protein; vii) at the 3′ end of the polynucleotide encoding the Class 2 CRISPR protein, between the stop codon and poly(A) termination site; or viii) any combination of (i)-(vi). In some embodiments, multiple copies of the self-inactivating segment are located in any combination of the foregoing locations, provided the self-inactivating segment is complementary to or identical to the targeting sequence of the gRNA. It will be understood that, depending on the design of the SIRV, a self-inactivating segment can be incorporated into the construct or it can be a sequence of nucleotides selected based on the presence of a PAM and a sequence downstream of the PAM that already exists within the components of the SIRV polynucleotide that is complementary to or is identical to the targeting sequence of the gRNA; the components being, e.g., promoters, the sequence encoding the Class 2 Type V protein, Kozak sequence, introns, etc. In some embodiments, an AAV comprises the SIRV construct comprising the foregoing one or more self-inactivating segments. Schematics of such configurations are presented in FIGS. 44-47 and 77, in which self-inactivating segments flank the sequence encoding the CRISPR nuclease. It is understood by one of skill in the art that in the context of an episomal form of an SIRV in a transfected or transduced cell, the foregoing configurations are in reference to the encoding strand.

a. Selective Use of PAM Sequences

A first designed approach of the SIRV constructs of the present disclosure utilizes a polynucleotide wherein the polynucleotide encodes a single gRNA comprising a targeting sequence complementary to a target nucleic acid and that also targets one or more self-inactivating segments incorporated into the SIRV polynucleotide. In some embodiments, the self-inactivating segments comprise a sequence identical to that of the encoded targeting sequence of the guide, as well as a PAM sequence (separated by a single intervening nucleotide) in the polynucleotide, wherein the PAM sequence proximal to the self-inactivating segment is different from the PAM sequence of the target nucleic acid intended for modification by the CRISPR protein-gRNA complex (RNP). In some embodiments, the polynucleotide comprises a single-stranded DNA transgene for incorporation into a viral particle, such as an adeno-associated virus (AAV); embodiments of which are described more fully, below. It will be understood that in this design, when incorporated into an AAV that is transduced into a target cell to be modified, the targeting sequence of the gRNA of the RNP binds to the anti-sense strand of the double-stranded episomal form in the transduced or transfected cell rather than the self-inactivating segment. As described more fully, below, the PAM of the self-inactivating segment promotes less efficient binding and cleavage and/or a lower rate of cleavage of the self-inactivating segment by the RNP compared to the PAM 5′ and adjacent to the target nucleic acid of the cell to be modified. It will be further understood that because the binding and cleavage of the self-inactivating segment by the RNP of the expressed CRISPR nuclease and gRNA is less efficient compared to that of the target nucleic acid, there can be a difference between the timing of the cleavage and/or the rate of cleavage of the respective sequences; i.e., a higher percentage of the target nucleic acid can be cleaved and edited before the cleavage of the self-inactivating segment that results in the inability to continue to transcribe the CRISPR components of the polynucleotide of the SIRV construct. In other embodiments, the self-inactivating segment incorporated into the polynucleotide is the complement of the targeting segment of the encoded targeting sequence and the selected less-efficient PAM such that the targeting sequence of the gRNA of the RNP binds to the self-inactivating segment of the polynucleotide rather than the anti-sense strand in the double-stranded episomal form created intracellularly.

In a variation of the foregoing, the present disclosure provides SIRV constructs comprising two gRNA wherein the targeting sequence of the first gRNA is complementary to a target nucleic acid at a first location in a cell to be modified and the targeting sequence of the second gRNA is complementary to both a target nucleic acid at a second location (in order to effect a dual-cut of the target nucleic acid) and is also complementary to the self-inactivating segments of the construct. In some embodiments of the foregoing, the self-inactivating segments are linked to a less-efficient PAM relative to the PAM of the target nucleic acid of the second cut such that there can be a difference between the timing of the cleavage of the target nucleic acid in a cell to be modified and the cleavage and inactivation of the CRISPR components of the SIRV.

In some embodiments, the encoded Class 2 Type V CRISPR protein is selected from the group consisting of Cas12a (Cpf1), Cas12b (C2c1), Cas12c (C2c3), Cas12d (CasY), Cas12e (CasX), Cas12f, Cas12g, Cas12h, Cas12i, Cas12j, Cas12k, Cas14, and Cas(D, and the encoded guide is that associated with the respective system; e.g., a Cas12a guide for a Cas12a nuclease. In some embodiments, the Class 2 Type V CRISPR protein is a CasX selected from the group consisting of SEQ ID NOS: 1-3, 49-321 and 2356-2488, or a sequence having at least at least about 80%, at least about 85%, at least about 90%, or at least about 95%, or at least about 95%, or at least about 96%, or at least about 97%, or at least about 98%, or at least about 99% sequence identity thereto, and the gRNA comprises a scaffold having a sequence of SEQ ID NOS: 2101-2331,3992-3995, or 4028 as set forth in Table 2, or a sequence having at least at least about 80%, at least about 85%, at least about 90%, or at least about 95%, or at least about 95%, or at least about 96%, or at least about 97%, or at least about 98%, or at least about 99% sequence identity thereto, and the gRNA further comprises a targeting sequence complementary to the target nucleic acid of the cell to be modified and to the self-inactivating segment or its complement. In a particular embodiment, the Class 2 Type V CRISPR protein is a CasX selected from the group consisting of SEQ ID NOS: 72-321 and 2356-2488 as set forth in Table 5, or a sequence having at least at least about 80%, at least about 85%, at least about 90%, or at least about 95%, or at least about 95%, or at least about 96%, or at least about 97%, or at least about 98%, or at least about 99% sequence identity thereto, and the gRNA comprises a scaffold having a sequence of SEQ ID NOS: 2101-2331, 3992-3995, or 4028, as set forth in Table 2, or a sequence having at least at least about 80%, at least about 85%, at least about 90%, or at least about 95%, or at least about 95%, or at least about 96%, or at least about 97%, or at least about 98%, or at least about 99% sequence identity thereto, and the gRNA further comprises a targeting sequence complementary to the target nucleic acid of the cell to be modified and to the self-inactivating segment or its complement. In another particular embodiment, the Class 2 Type V CRISPR protein is a CasX of SEQ ID NOS: 138 or 145 as set forth in Table 5, or a sequence having at least at least about 80%, at least about 85%, at least about 90%, or at least about 95%, or at least about 95%, or at least about 96%, or at least about 97%, or at least about 98%, or at least about 99% sequence identity thereto, and the gRNA comprises a scaffold having a sequence of SEQ ID NOS: 2296 as set forth in Table 2, or a sequence having at least at least about 80%, at least about 85%, at least about 90%, or at least about 95%, or at least about 95%, or at least about 96%, or at least about 97%, or at least about 98%, or at least about 99% sequence identity thereto, and the gRNA further comprises a targeting sequence complementary to the target nucleic acid of the cell to be modified and to the self-inactivating segment or its complement. In another particular embodiment, the Class 2 Type V CRISPR protein is a CasX of SEQ ID NOS: 138 or 145 as set forth in Table 5, or a sequence having at least at least about 80%, at least about 85%, at least about 90%, or at least about 95%, or at least about 95%, or at least about 96%, or at least about 97%, or at least about 98%, or at least about 99% sequence identity thereto, and the gRNA comprises a scaffold having a sequence of SEQ ID NOS: 4028 as set forth in Table 2, or a sequence having at least at least about 80%, at least about 85%, at least about 90%, or at least about 95%, or at least about 95%, or at least about 96%, or at least about 97%, or at least about 98%, or at least about 99% sequence identity thereto, and the gRNA further comprises a targeting sequence complementary to the target nucleic acid of the cell to be modified and to the self-inactivating segment or its complement. Upon the binding and cleavage of the self-inactivating segment by the RNP (which, in the case of a cell transfected with the SIRV or transduced with a viral particle comprising the SIRV, would be incorporated in a double-stranded episome in the cell), the functional expression of one or more of the CRISPR components encoded by the polynucleotide is diminished or prevented; e.g., the Class 2, Type V nuclease or the gRNA. The characteristics and properties of the CRISPR nucleases, gRNA and targeting sequences, and their ability to edit target nucleic acid, are described more fully, below.

While it is established that the canonical PAM for wild-type CasX (and for several CasX variants) is TTC, the binding preferences for the non-canonical PAM sequences can vary. For example, while the binding preference for the PAM by CasX 491 and 515 is in the order TTC>ATC>CTC>GTC>TTT>GTT, in a 5′ to 3′ orientation, for CasX 668 and 672 the order is TTC>CTC>ATC>GTC>TTT>GTT. As used herein, in relation to PAM sequences “binding preference” means that the binding affinity for the PAM sequence is stronger than that of a different PAM sequence. Accordingly, the PAM sequence of the self-inactivating segment is chosen to take advantage of the preferential PAM binding. In some embodiments, wherein the PAM sequence adjacent to the target nucleic acid of the cell to be modified is TTC and the sequence encoding a CasX having a preference for TTC PAM is incorporated into the transgene of the SIRV, the SIRV is designed to utilize a PAM sequence in the one or more self-inactivating segments selected from the group consisting of ATC, CTC, and GTC, which are less efficient in promoting the binding and cleavage of the adjacent nucleic acid of the self-inactivating segment by the RNP. In other embodiments, wherein the PAM sequence adjacent to the target nucleic acid of the cell to be modified is ATC, then if the CasX preferential PAM or order is TTC>ATC>CTC>GTC>TTT>GTT, the PAM sequence utilized in the one or more self-inactivating segments is CTC, GTC, TTT, or GTT which are less efficient in promoting the binding and cleavage of the adjacent nucleic acid of the self-inactivating segment by the RNP. It has been discovered that in the generation of CasX variants, in some cases, select CasX variants preferentially or more efficiently bind PAM sequences in the order ATC>CTC>GTC>TTC>TTT>GTT, GTC>ATC>CTC>TTC>TTT>GTT, or CTC>ATC>GTC>TTC>TTT>GTT. It will be understood, therefore, that a CasX variant with a different PAM preference can be utilized in the SIRV constructs, in which case the same principles described above would apply, but the choice of the PAM utilized in the self-inactivating construct would be different. For example, the PAM preference for CasX variant 533 is in the order ATC>CTC>GTC>TTC>TTT>GTT, in a 5′ to 3′ orientation. In such a case, if the PAM sequence of the target nucleic acid was ATC and CasX 533 was utilized in the SIRV construct, the PAM sequence of the self-inactivating segment would be chosen from CTC, GTC, TTC, TTT, or GTT, which are less efficient in promoting the binding and cleavage of the adjacent nucleic acid self-inactivating segment by the RNP. Similarly, in those cases wherein the PAM sequence adjacent to the target nucleic acid of the cell to be modified is GTC and the encoded the CasX variant encoded in the SIRV of the system preferentially binds PAM sequences in the order GTC>ATC>CTC>TTC>TTT>GTT, then the PAM sequence of the one or more self-inactivating segments is selected from the group consisting of ATC, CTC, TTC, TTT, and GTT which are less efficient in promoting the binding and cleavage of the adjacent nucleic acid self-inactivating segment by the RNP. It will be appreciated that so long as a CasX is chosen for incorporation in the SIRV wherein a PAM sequence with a lower binding potential than the PAM sequence of the target nucleic is available, the self-inactivating segment can be appropriately designed with a PAM sequence to confer the desired differential inactivation (e.g., slower rate of binding and/or cleavage) of the resulting construct. It will be further appreciated that the self-inactivating segment sequences of the SIRV can be designed that comprises a sequence that is the complement to the targeting sequence of the gRNA, such that the anti-sense strand of the subsequently formed double-stranded episome would comprise a sequence identical to the targeting sequence with the corresponding PAM and the double-stranded episomal sequence would be cleaved by the RNP of the CasX and gRNA encoded by the SIRV.

In some embodiments of an SIRV, if the PAM sequence of the target nucleic acid of the cell to be modified is TTC, and the PAM preference of the Class 2 Type V protein is TTC, then the PAM sequence of the one or more self-inactivating segments is selected from the group consisting of ATC, CTC, TTT, GTT, and GTC.

In some embodiments of an SIRV, if the PAM sequence of the target nucleic acid of the cell to be modified is ATC and the PAM preference of the Class 2 Type V protein is TTC, then the PAM sequence of the one or more self-inactivating segments is selected from the group consisting of CTC, TTT, GTT, and GTC.

In some embodiments of an SIRV, if the PAM sequence of the target nucleic acid of the cell to be modified is CTC and the PAM preference of the Class 2 Type V protein is TTC, then the PAM sequence of the one or more self-inactivating segments is TTT, GTT, ATC, or GTC.

In some embodiments of an SIRV, if the PAM sequence of the target nucleic acid of the cell to be modified is ATC and the PAM preference of the Class 2 Type V protein is ATC, then the PAM sequence of the one or more self-inactivating segments is selected from the group consisting of TTC, CTC, TTT, GTT, and GTC.

In some embodiments of an SIRV, if the PAM sequence of the target nucleic acid of the cell to be modified is CTC and the PAM preference of the Class 2 Type V protein is ATC, then the PAM sequence of the one or more self-inactivating segments is selected from the group consisting of TTC, TTT, GTT, and GTC.

In some embodiments of an SIRV, if the PAM sequence of the target nucleic acid of the cell to be modified is GTC and the PAM preference of the Class 2 Type V protein is ATC, then the PAM sequence of the one or more self-inactivating segments is TTC, TTT, CTC, or GTT.

In some embodiments of an SIRV, if the PAM sequence of the target nucleic acid of the cell to be modified is GTC and the PAM preference of the Class 2 Type V protein is GTC, then the PAM sequence of the one or more self-inactivating segments is selected from the group consisting of ATC, CTC, TTT, GTT, and TTC.

In some embodiments of an SIRV, if the PAM sequence of the target nucleic acid of the cell to be modified is GTC and the PAM preference of the Class 2 Type V protein is ATC, then the PAM sequence of the one or more self-inactivating segments is selected from the group consisting of TTC, TTT, GTT, and CTC.

In some embodiments of an SIRV, if the PAM sequence of the target nucleic acid of the cell to be modified is CTC and the PAM preference of the Class 2 Type V protein is GTC, then the PAM sequence of the one or more self-inactivating segments is TTC, TTT, ATC, or GTT.

In some embodiments of an SIRV, if the PAM sequence of the target nucleic acid of the cell to be modified is CTC and the PAM preference of the Class 2 Type V protein is CTC, then the PAM sequence of the one or more self-inactivating segments is selected from the group consisting of ATC, GTC, TTT, GTT, and TTC.

In some embodiments of an SIRV, if the PAM sequence of the target nucleic acid of the cell to be modified is ATC and the PAM preference of the Class 2 Type V protein is CTC, then the PAM sequence of the one or more self-inactivating segments is selected from the group consisting of GTC, TTT, GTT, and TTC.

In some embodiments of an SIRV, if the PAM sequence of the target nucleic acid of the cell to be modified is GTC and the PAM preference of the Class 2 Type V protein is CTC, then the PAM sequence of the one or more self-inactivating segments is TTC, TTT, ATC, or GTT.

In the foregoing embodiments of differential PAM utilization, the cleavage of the self-inactivating segments (in the double-stranded episome) by the RNP is at least about 10%, at least about 20%, at least about 30%, at least about 40%, at least about 50%, at least about 60%, at least about 70%, at least about 80%, or at least about 90% less than the cleavage of the target nucleic acid in a timed in vitro cell-based assay, when assayed under comparable conditions. In some embodiments, the cleavage of the self-inactivating segments (in the double-stranded episome of a cell) by the RNP to achieve 90% cleavage is delayed, relative to the time to achieve 90% editing of a target nucleic acid in a cell, by at least about 1 day, at least about 2 days, at least about 3 days, at least about 4 days, at least about 5 days, at least about 6 days, at least about 7 days, at least about 8 days, or at least about 9 days, when assayed in an in vitro assay under comparable conditions. In other embodiments, when assayed for rate of cleavage, cleavage of the self-inactivating segments by the RNP has a k_{cleave} rate that is at least about 2-fold, at least about 4-fold, at least about 5-fold, at least about 6-fold, at least about 7-fold, at least about 8-fold, at least about 9-fold, or at least about 10-fold less than the k_{cleave} rate of the target nucleic acid in an in vitro cell-based assay, when assayed under comparable conditions. Exemplary assays, as well as constructs utilized to demonstrate these properties are provided in the Examples, below.

b. Self-Limiting Segment with Non-Complementary Bases

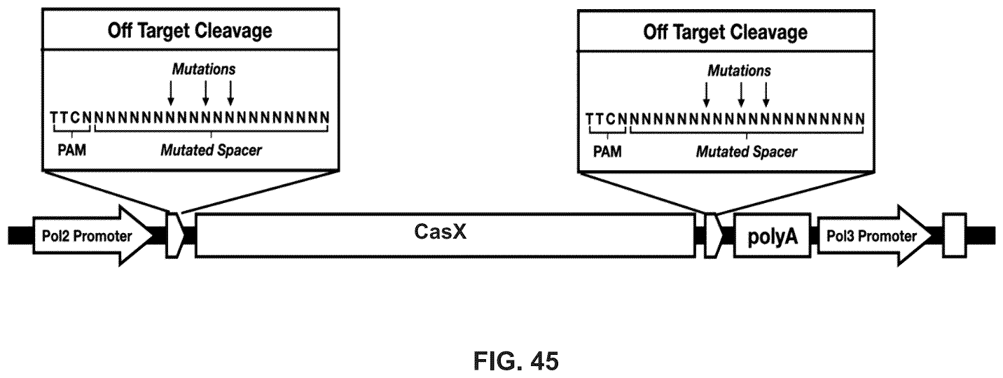

In another designed approach of the SIRV constructs of the present disclosure, the design utilizes a single gRNA and one or more self-inactivating segments in the polynucleotide, wherein the one or more self-inactivating segments of the polynucleotide are capable of being bound and cleaved by the RNP (in the double-stranded episome stage in a cell), but each have between 1 to 5 bases, between 1 to 4 bases, or between 1 to 3 bases that are mismatches and are not complementary to corresponding positions in the targeting sequence of the first gRNA such that the RNP exhibits less efficient binding and cleavage or reduced rate of cleavage of the self-inactivating segment compared to the binding and cleavage of the target nucleic acid. In some embodiments of the foregoing, the base differences of the one or more self-inactivating segments are relative to positions that are 3′ to the fourth nucleotide of the targeting sequence of the gRNA; positions that are more critical for the action of the CRISPR nuclease, such that the binding affinity of the targeting sequence of the guide in the RNP to the self-inactivating segment is reduced compared to the binding affinity of the targeting sequence of the gRNA to the target nucleic acid. A schematic representation of one design of the polynucleotide and the location(s) of the self-inactivating segments is shown in FIG. 45. In another embodiment, the self-inactivating segments of the polynucleotide comprise a sequence that is identical (except for the bases that are mismatched) to that of the targeting sequence of the gRNA such that the anti-sense strand of the episomal form is bound by the RNP. It will be understood that as a result of the base mismatches, the binding and cleavage and/or the rate of cleavage of the self-inactivating segment will be reduced compared to that of the target nucleic acid in, for example, an assay where both sequences are accessible by the RNP. In some embodiments of the design, and, as previously described above, an additional feature that can be utilized is that the PAM sequence utilized in the one or more self-inactivating segments is different from the PAM sequence of the target nucleic acid of the cell to be modified in order to promote less efficient binding and cleavage or cleavage rate of the self-inactivating segment by the RNP compared to the PAM of the target nucleic acid of the cell to be modified.

In some embodiments, the cleavage of the self-inactivating segments by the RNP with mismatched bases is at least about 10%, at least about 20%, at least about 30%, at least about 40%, at least about 50%, at least about 60%, at least about 70%, at least about 80%, or at least about 90% less than the cleavage of the target nucleic acid by the RNP in a timed in vitro cell-based assay, when assayed under comparable conditions. In some embodiments, the cleavage of the self-inactivating segments (in the double-stranded episome of a cell) by the RNP to achieve 90% cleavage is delayed, relative to the time to achieve 90% editing of a target nucleic acid by the RNP in a cell, by at least about 1 day, at least about 2 days, at least about 3 days, at least about 4 days, at least about 5 days, at least about 6 days, at least about 7 days, at least about 8 days, or at least about 9 days, when assayed in an in vitro assay under comparable conditions. When assayed for rate of cleavage, cleavage of the self-inactivating segments by the RNP has a k_{cleave} rate that is at least about 2-fold, at least about 4-fold, at least about 5-fold, at least about 6-fold, at least about 7-fold, at least about 8-fold, at least about 9-fold, or at least about 10-fold less than the k_{cleave} rate of the target nucleic acid by the RNP in an in vitro cell-based assay, when assayed under comparable conditions. Exemplary assays utilized to demonstrate these properties are provided in the Examples, below. In the embodiments of the foregoing design, the one or more self-inactivating segments can be located within the transgene polynucleotide at the locations previously described, and the CRISPR nuclease, the gRNA, and the regulatory and accessory elements incorporated in the transgene can be selected from the embodiments described herein.

In some embodiments, the disclosure provides SIRV constructs in which self-limiting segments with non-complementary bases can be combined with the selective use of less-efficient PAM sites described, supra.

c. Second gRNA Specific for Self-Inactivating Segments

In other design approaches, the SIRV polynucleotides of the present disclosure are designed to encode a second gRNA that specifically targets the self-inactivating segments rather than the target nucleic acid to be modified. An important feature of the design is that the second gRNA comprises a scaffold that is designed to promote equivalent or less efficient binding of the self-inactivating segment compared to the binding and cleavage of the target nucleic acid by an RNP of the Class 2 Type V CRISPR protein and the first gRNA. In some embodiments, the second gRNA scaffold has a sequence identical to that of the first gRNA. In other embodiments, the second gRNA scaffold has a sequence different to that of the first gRNA. In some cases of the foregoing, the SIRV polynucleotide encodes a second guide scaffold comprising a sequence selected from the group consisting of SEQ ID NOS: 2101-2331 and 3992-3995 and encodes a first guide scaffold comprising a sequence selected from SEQ ID NOS: 2276-2296 corresponding to guide variant 215 to 235 as set forth in Table 2, or a sequence with at least 70% sequence identity thereto. In a particular embodiment, the SIRV polynucleotide encodes a second guide scaffold comprising the sequence of SEQ ID NO: 2238 (guide scaffold 174) and encodes a first guide scaffold comprising the sequence of SEQ ID NO: 2296 (guide scaffold 235). In another particular embodiment, the SIRV polynucleotide encodes a second guide scaffold comprising the sequence of SEQ ID NO: 2238 (guide scaffold 174), or a sequence with at least 70% sequence identity thereto, and encodes a first guide scaffold comprising the sequence of SEQ ID NO: 4028 (guide scaffold 316), or a sequence with at least 70% sequence identity thereto.

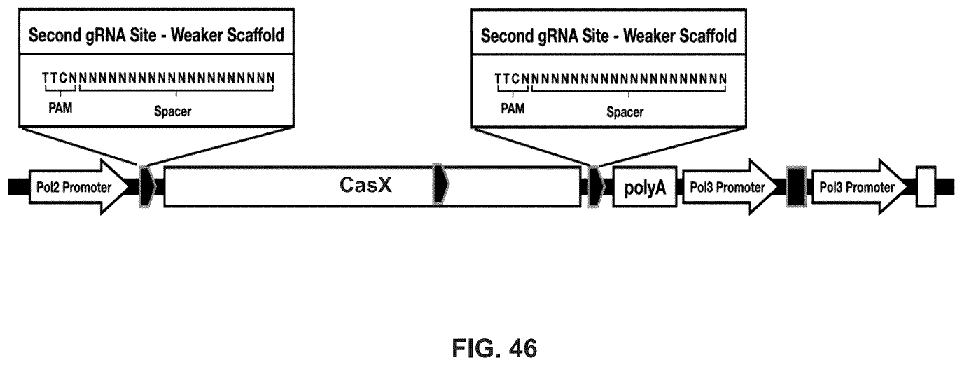

The differential activity of the gRNA on the ability of the nuclease-gRNA complex to edit nucleic acids is demonstrated in the Examples, where constructs with guide scaffolds 231-236 (SEQ ID NOS: 2288-2293) edited at a higher level compared to constructs with guide 174 (SEQ ID NO: 2238). A schematic representation of one design of the polynucleotide and the location(s) of the self-inactivating segments is shown in FIG. 46. It will be understood that because the binding and cleavage or cleavage rate of the RNP targeting the self-inactivating segment is less efficient compared to that of the target nucleic acid, there is a temporal difference between the timing of cleavage and/or a reduced rate of cleavage compared to an RNP comprising the first gRNA targeting the target nucleic acid of the cell.

In some embodiments of the foregoing alternative designs, the disclosure provides SIRV comprising a polynucleotide comprising sequences for components selected from i) a packaging component; ii) a sequence encoding a Class 2 CRISPR protein; iii) a first promoter operably linked to the sequence encoding the Class 2 CRISPR protein; iv) a sequence encoding a first guide RNA (gRNA) comprising a targeting sequence that is complementary to a target nucleic acid of a cell to be modified; v) a second promoter sequence operably linked to the sequence encoding the first gRNA; vi) a sequence encoding a second gRNA having a scaffold sequence identical to the scaffold sequence of the first gRNA and having a targeting sequence that has a lower binding affinity to one or more self-limited segments utilized in the polynucleotide compared to the binding affinity of the targeting sequence of the first gRNA to the target nucleic acid of the cell to be modified; vii) a sequence encoding a second gRNA having a scaffold sequence different from the scaffold sequence of the first gRNA and having a targeting sequence that is complementary to one or more self-limited segments utilized in the polynucleotide, wherein the second gRNA promotes editing and/or cleavage by an RNP of the Class 2 CRISPR protein and the second gRNA that is equal to or less efficient compared to an RNP of the Class 2 CRISPR protein and the first gRNA; viii) a third promoter sequence operably linked to the sequence encoding the second gRNA; and ix) one or more self-inactivating segments of the polynucleotide comprising a protospacer adjacent motif (PAM) sequence and a polynucleotide sequence capable of being bound and cleaved by a ribonuclear protein complex (RNP) of the Class 2 CRISPR protein and the second gRNA. The location of the self-limiting segments in the transgene can be at locations previously described. In the embodiments of these foregoing designs, the cleavage of the self-inactivating segments (in the double-stranded episome of a cell) by the RNP comprising the second gRNA is at least about 10%, at least about 20%, at least about 30%, at least about 40%, at least about 50%, at least about 60%, at least about 70%, at least about 80%, or at least about 90% less than the cleavage of the target nucleic acid by the RNP comprising the first gRNA in a timed in vitro cell-based assay, when assayed under comparable conditions. In some embodiments, the cleavage of the self-inactivating segments by the RNP comprising the second gRNA to achieve 90% cleavage is delayed, relative to the time to achieve 90% editing of a target nucleic acid by the RNP comprising the first gRNA in a cell, by at least about 1 day, at least about 2 days, at least about 3 days, at least about 4 days, at least about 5 days, at least about 6 days, at least about 7 days, at least about 8 days, or at least about 9 days, when assayed in an in vitro assay under comparable conditions. When assayed for rate of cleavage, cleavage of the self-inactivating segments by the RNP by the RNP comprising the second gRNA has a k_{cleave} rate that is at least about 2-fold, at least about 4-fold, at least about 5-fold, at least about 6-fold, at least about 7-fold, at least about 8-fold, at least about 9-fold, or at least about 10-fold less than the k_{cleave} rate of the target nucleic acid by the RNP comprising the first gRNA in an in vitro cell-based assay, when assayed under comparable conditions. Exemplary assays utilized to demonstrate these properties are provided in the Examples, below. In the embodiments of the foregoing design, the one or more self-inactivating segments can be located within the transgene polynucleotide at the locations previously described, and the CRISPR nuclease, the gRNA, and the regulatory and accessory elements incorporated in the transgene can be selected from the embodiments described herein.

d. Second gRNA and Less-Efficient Promoter for Self-Inactivating Segments

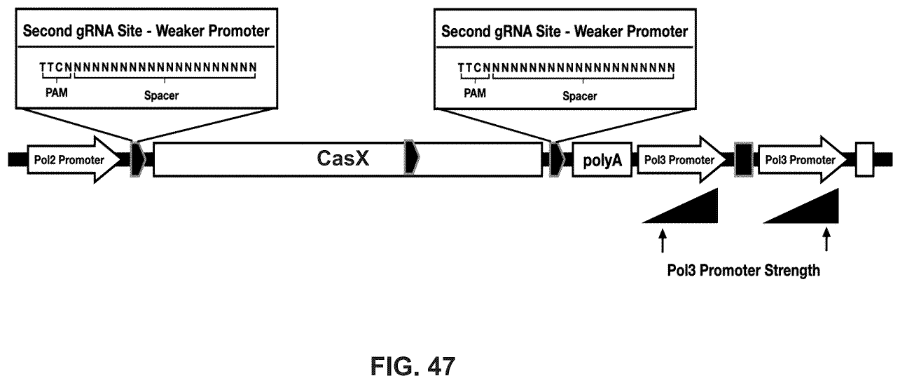

In another design approach, the polynucleotide of the SIRV of the present disclosure is designed to encode a second gRNA that specifically targets the incorporated self-inactivating segments wherein the second gRNA is under the control of a third, less efficient pol III promoter compared to the second promoter controlling the first gRNA. It will be understood that because the third promoter is less efficient at initiating transcription of the second gRNA, the expression of the second gRNA is delayed or is reduced compared to the first gRNA such that the target nucleic acid of the cells can be modified by the RNP of the nuclease and the first gRNA before the polynucleotide of the SIRV is inactivated by the RNP of the second gRNA and nuclease. In some embodiments, the third promoter is selected from the group consisting of truncated U6, sequence variants of U6, mini U6, truncated 7SK, sequence variants of 7SK, truncated H1, sequence variants of H1, bidirectional H1, bidirectional U6, bidirectional 7SK, and bidirectional U6, 5S promoter, and Adenovirus 2 (Ad2) VAI promoter and truncated or sequence variants thereof. Native U6, 7SK and H1 are generally considered strong promoters and would, therefore, be appropriate for use with the first gRNA targeting the target nucleic acid. Representative examples of promoters contemplated for use as the third promoter include, but are not limited to, the sequences of SEQ ID NOS: 494-513 and 2688-2708 as set forth in Table 25, and sequences having at least about 80%, at least about 90%, or at least about 95%, or at least about 95%, or at least about 96%, or at least about 97%, or at least about 98%, or at least about 99% sequence identity thereto. In some embodiments, the scaffold of the second gRNA is identical to that of the first gRNA. In other embodiments, the scaffold of the second gRNA is different and is less efficient compared to the first gRNA; e.g., gRNA 174 (SEQ ID NO: 2238) is less efficient compared to gRNA 235 (SEQ ID NO: 2296). In some embodiments, the SIRV polynucleotide encodes a second guide scaffold comprising a sequence selected from the group consisting of SEQ ID NO: 2101-2238 and 3992-3995 and encodes a first guide scaffold comprising a sequence selected from SEQ ID NOS: 2276-2296 corresponding to guide variant scaffolds 215 to 235. In a particular embodiment, the SIRV polynucleotide encodes a second guide scaffold comprising the sequence of guide scaffold 174 (SEQ ID NO: 2238) and encodes a first guide scaffold comprising the sequence of guide scaffold 235 (SEQ ID NO: 2296). A schematic representation of one design of the polynucleotide and the locations of the self-inactivating segments is shown in FIG. 47. In the embodiments of the design, the cleavage of the self-inactivating segments by the RNP comprising the second gRNA is at least about 10%, at least about 20%, at least about 30%, at least about 40%, at least about 50%, at least about 60%, at least about 70%, at least about 80%, or at least about 90% less than the cleavage of the target nucleic acid by the RNP comprising the first gRNA in a timed in vitro cell-based assay, when assayed under comparable conditions. In some embodiments, the cleavage of the self-inactivating segments (in the double-stranded episome of a cell) by the RNP to achieve 90% cleavage is delayed, relative to the time to achieve 90% editing of a target nucleic acid in a transduced or transfected cell, by at least about 1 day, at least about 2 days, at least about 3 days, at least about 4 days, at least about 5 days, at least about 6 days, at least about 7 days, at least about 8 days, or at least about 9 days, when assayed in an in vitro assay under comparable conditions. When assayed for rate of cleavage, cleavage of the self-inactivating segments by the RNP has a k_{cleave} rate that is at least about 2-fold, at least about 4-fold, at least about 5-fold, at least about 6-fold, at least about 7-fold, at least about 8-fold, at least about 9-fold, or at least about 10-fold less than the k_{cleave} rate of the target nucleic acid in an in vitro cell-based assay, when assayed under comparable conditions. Exemplary assays utilized to demonstrate these properties are provided in the Examples, below. In the embodiments of the foregoing design, the one or more self-inactivating segments can be located within the transgene polynucleotide at the locations previously described, and the CRISPR nuclease, the gRNA, and the regulatory and accessory elements incorporated in the transgene can be selected from the embodiments described herein.

In some embodiments of the foregoing design, the disclosure provides SIRV compositions comprising a polynucleotide comprising sequences for components selected from the group consisting of: i) a packaging component; ii) a sequence encoding a Class 2 CRISPR protein; iii) a first promoter operably linked to the sequence encoding the Class 2 CRISPR protein; iv) a sequence encoding a first guide RNA (gRNA) scaffold and a targeting sequence that is complementary to a target nucleic acid of a cell to be modified; v) a second promoter sequence operably linked to the sequence encoding the first gRNA; vi) a sequence encoding a second guide RNA (gRNA) having a targeting sequence different from the targeting sequence of the first gRNA; vii) a third promoter sequence operably linked to the sequence encoding the second gRNA, wherein the third promoter has a sequence different from the sequence of the second promoter; and viii) one or more self-inactivating segments of the polynucleotide comprising a protospacer adjacent motif (PAM) sequence and a polynucleotide sequence capable of being bound and cleaved by a ribonuclear protein complex (RNP) of the Class 2 CRISPR protein and the second gRNA.

e. Combinations of SIRV Designs

In some cases, the alternative designs of the SIRV can be combined to further enhance or tailor the degree or onset of inactivation of the expressed CRISPR components in the cell. As detailed, supra, four of the SIRV design approaches (summarized in a general, non-limiting way) are: 1) use of alternative, less efficient PAM sites adjacent to the self-inactivating segment; 2) use of non-complementary bases in the self-inactivating segment (relative to the targeting sequence of the gRNA); 3) incorporation of a second gRNA in the SIRV with a different, less-efficient scaffold or having an identical scaffold but use of a targeting sequence with lower affinity to the self-inactivating segment compared to the first gRNA targeting the target nucleic acid; and 4) incorporation of a second gRNA in the SIRV with a different, less-efficient promoter compared to the promoter of the first gRNA targeting the target nucleic acid. The disclosure contemplates SIRV designs using any and all combinations of the foregoing. In some embodiments, a SIRV construct design incorporates use of an alternative, less efficient PAM sites adjacent to the self-inactivating segment in combination with non-complementary bases in the self-inactivating segment (design #1 and #2). In other embodiments, a SIRV construct design incorporates use of an alternative, less efficient PAM sites adjacent to the self-inactivating segment in combination with a second gRNA in the SIRV with a different, less-efficient scaffold or having an identical scaffold but use of a targeting sequence with lower affinity to the self-inactivating segment (design #1 and #3). In other embodiments, a SIRV construct design incorporates use of an alternative, less efficient PAM site adjacent to the self-inactivating segment in combination with a second gRNA in the SIRV with a different, less-efficient promoter compared to the promoter of the first gRNA (design #1 and #4). In other embodiments, a SIRV construct design incorporates use of non-complementary bases in the self-inactivating segment in combination with a different, less-efficient scaffold or having an identical scaffold but use of a targeting sequence with lower affinity to the self-inactivating segment (design #2 and #3). In other embodiments, a SIRV construct design incorporates use of non-complementary bases in the self-inactivating segment in combination with a second gRNA in the SIRV with a different, less-efficient promoter compared to the promoter of the first gRNA (design #2 and #4). In other embodiments, a SIRV construct design incorporates a second gRNA in the SIRV with a different, less-efficient scaffold or having an identical scaffold but use of a targeting sequence with lower affinity to the self-inactivating segment in combination with a second gRNA in the SIRV with a different, less-efficient promoter compared to the promoter of the first gRNA (design #3 and #4). In other embodiments, a SIRV construct design incorporates three of the foregoing designs in any combination; e.g., #1, #2, and #3, or #2, #3, and #4, or #1, #3, and #4. In still other embodiments, a SIRV construct design incorporates four of the foregoing designs. It will be appreciated by one of skill in the art that by using constructs having multiple designs, the degree or onset of inactivation of the expressed CRISPR components can be tailored to achieve the desired outcome of the desired modification of the target nucleic acid and inactivation of the SIRV.

III. Use of Class 2 Type V CRISPR to Permit Inclusion of Additional Components in the SIRV and siAAV Transgene

Provided herein are Class 2 Type V systems, which due to their smaller size, permit the inclusion of additional sequence space in an SIRV transgene. These SIRV can be delivered to cells by transduction and used in the making and packaging of self-inactivating AAV (siAAV) particles.

Wild-type AAV is a small, single-stranded replication-defective DNA virus belonging to the parvovirus family. The wild-type AAV genome is made up of two genes that encode four replication proteins and three capsid proteins, respectively, and is flanked on either side by inverted terminal repeats (ITRs) having 130-145 nucleotides that fold into a hairpin shape important for replication. The virion is composed of three capsid proteins, Vp1, Vp2, and Vp3, produced in a 1:1:10 ratio from the same open reading frame but from differential splicing (Vp1) and alternative translational start sites (Vp2 and Vp3, respectively). The cap gene produces an additional, non-structural protein called the Assembly-Activating Protein (AAP). This protein is produced from ORF2 and is essential for the capsid-assembly process. The capsid forms a supramolecular assembly of approximately 60 individual capsid protein subunits into a non-enveloped, T-1 icosahedral lattice capable of protecting the AAV genome.

Wild-type AAV is capable of transducing nearly every cell type in the human body. Typically, when producing a recombinant AAV vector, the sequence between the two ITRs is replaced with one or more sequences of interest (e.g., a transgene), and the Rep and Cap sequences are provided in trans, making the ITRs the only viral DNA that remains in the vector. The resulting recombinant AAV vector genome construct comprises two cis-acting 130 to 145-nucleotide ITRs flanking an expression cassette encoding the transgene sequences of interest, providing at least 4.7 kb or more for packaging of foreign DNA that can include a transgene, one or more promoters and accessory elements, such that the total size of the vector is below 5 to 5.2 kb, which is compatible with packaging within the AAV capsid (it being understood that as the size of the construct exceeds this threshold, the packaging efficiency of the vector decreases). The transgene may be used to correct or ameliorate gene deficiencies in the cells of a subject. However, in the context of CRISPR-mediated gene editing, the size limitation of the expression cassette is a challenge for most CRISPR systems due to the size of the nucleases.

As provided herein, the smaller Class 2, Type V proteins, and gRNA contemplated for inclusion in the vector, permits inclusion of additional or larger components that can be packaged into a self-inactivating AAV (siAAV) or other viral particle. In some embodiments, the disclosure provides an siAAV comprising components of a Class 2 Type V CRISPR system. In some embodiments, the Class 2 CRISPR protein of the siAAV comprises a Type V protein selected from the group consisting of Cas12a (Cpf1), Cas12b (C2c1), Cas12c (C2c3), Cas12d (CasY), Cas12e (CasX), Cas12f, Cas12g, Cas12h, Cas12i, Cas12j, Cas12k, Cas14, and Cas(D, and the associated guide RNA of the respective system. In a particular embodiment, the CRISPR protein is a CasX, wherein the CasX comprises a sequence selected from the group consisting of SEQ ID NOS: 1-3, 49-321 and 2356-2488, or a sequence having at least about 85%, at least about 90%, at least about 91%, at least about 92%, at least about 93%, at least about 94%, at least about 95%, at least about 96%, at least about 97%, at least about 98%, or at least about 99% sequence identity thereto. In some embodiments, the siAAV transgene comprises a first gRNA and, in some cases, a second gRNA comprising a scaffold sequence selected from the group consisting of SEQ ID NOS: 2101-2331 and 3992-3995, or a sequence having at least 85%, at least 90%, at least 95%, at least 95%, at least 96%, at least 97%, at least 98% identity thereto. In the foregoing embodiments, the gRNA further comprises a targeting sequence, wherein the targeting sequence has at least 15 to 30 nucleotides. The CasX protein and gRNA component embodiments contemplated for incorporation into the siAAV vectors of the disclosure are described more fully, below.

The smaller size of the Class 2, Type V proteins and gRNA contemplated for inclusion in the vector constructs permit inclusion of additional or larger components that can be packaged into a single viral particle, such as an siAAV. The AAV components of the siAAV of the disclosure may be created using AAV capsids and ITR derived from any of several AAV serotypes, including without limitation, AAV1, AAV2, AAV3, AAV4, AAV5, AAV6, AAV7, AAV8, AAV9, AAV10, AAV11, AAV12, AAV 44.9, AAV 9.45, AAV 9.61, AAV-Rh74, and AAVRh10, and modified capsids of these serotypes. In some embodiments, the AAV capsids utilized for the siAAV of the disclosure may be tissue-specific. In some embodiments, the siAAV capsid is of serotype 9 or of serotype 6, e.g., to target motor neurons and glia of the spinal cord. In some embodiments, the methods provide use of AAV9 or AAV6 for targeting of neurons via intraparenchymal brain injection. In some embodiments, the siAAV vector is derived from AAV9, e.g., delivered intravenously to penetrate the blood-brain barrier, and to drive gene expression in the nervous system via both neuronal and glial tropism of the vector. In other embodiments, the siAAV vector is derived from serotype 8, e.g., to deliver polynucleotides to retinal cells, liver, skeletal muscle and/or the heart. In other embodiments, the siAAV vector is derived from AAV serotype 2, e.g., to deliver polynucleotides to skeletal muscle, neurons, vascular smooth muscle cells, and/or hepatocytes. In other embodiments, the siAAV vector is derived from AAV-Rh10, e.g., to deliver polynucleotides to the CNS, lung, liver, muscle cells, and/or the heart. In some embodiments, an siAAV intended for use in muscle may comprise an AAV capsid of MyoAAV 1A1, MyoAAV 1A2, or MyoAAV 2A.

In some embodiments, the encoded CRISPR nuclease and gRNA sequences of the transgene incorporated into the siAAV are less than about 3100, about 3090, about 3080, about 3070, about 3060, about 3050, or less than about 3040 nucleotides in length. In other embodiments, the encoded CRISPR nuclease and gRNA sequences of the transgene incorporated into the siAAV are less than about 3040 to about 3100 nucleotides in length. Thus, in light of the total length of the expression cassette that can be packaged into an siAAV particle, in some embodiments, the polynucleotide sequences of the first promoter and the at least one accessory element have greater than at least about 1300, at least about 1350, at least about 1360, at least about 1370, at least about 1380, at least about 1390, at least about 1400, at least about 1500, at least about 1600 nucleotides, at least 1650, at least about 1700, at least about 1750, at least about 1800, at least about 1850, or at least about 1900 nucleotides in combined length. In other embodiments, the polynucleotide sequences of the first promoter and the at least one accessory element for incorporation into an SIRV for packaging into an siAAV have greater than at least about 1300 to at least about 1900 nucleotides in combined length. In one embodiment, the polynucleotide sequences of the first promoter and the at least one accessory element for incorporation into an SIRV for packaging into an siAAV have greater than 1314 nucleotides in combined length. In another embodiment, the polynucleotide sequences of the first promoter and the at least one accessory element for incorporation into the SIRV of an siAAV have greater than 1381 nucleotides in combined length. In other embodiments, the polynucleotide sequences of the first promoter, the second promoter and the at least one accessory element for incorporation into the SIRV for packaging into an siAAV have greater than at least about 1300, at least about 1350, at least about 1360, at least about 1370, at least about 1380, at least about 1390, at least about 1400, at least about 1500, at least about 1600 nucleotides, at least 1650, at least about 1700, at least about 1750, at least about 1800, at least about 1850, or at least about 1900 nucleotides in combined length. In other embodiments, the polynucleotide sequences of the first promoter, the second promoter and the at least one accessory element for incorporation into the SIRV for packaging into an siAAV have greater than at least about 1300 to at least about 1900 nucleotides in combined length. In other embodiments, the polynucleotide sequences of the first promoter, the second promoter, the third promoter and the at least one accessory element for incorporation into the SIRV for packaging into an siAAV have greater than at least about 1300 to at least about 1900 nucleotides in combined length. In one embodiment, the polynucleotide sequences of the first promoter, the second promoter, the third promoter and the at least one accessory element for incorporation into the SIRV for packaging into an siAAV have greater than 1314 nucleotides in combined length. In another embodiment, the polynucleotide sequences of the first promoter, the second promoter, the third promoter and the at least one accessory element for incorporation into the SIRV for packaging into an siAAV have greater than 1381 nucleotides in combined length. In still other embodiments, the polynucleotide sequences of the first promoter, the second promoter, and the two or more accessory elements for incorporation into the SIRV for packaging into an siAAV have greater than at least about 1300, at least about 1350, at least about 1360, at least about 1370, at least about 1380, at least about 1390, at least about 1400, at least about 1500, at least about 1600 nucleotides, at least 1650, at least about 1700, at least about 1750, at least about 1800, at least about 1850, or at least about 1900 nucleotides in combined length. In other embodiments, the polynucleotide sequences of the first promoter, the second promoter, and the two or more accessory elements for incorporation into the SIRV for packaging into an siAAV have greater than at least about 1300 to at least about 1900 nucleotides in combined length. In one embodiment, the polynucleotide sequences of the first promoter, the second promoter, and the two or more accessory elements for incorporation into the SIRV for packaging into an siAAV have greater than 1314 nucleotides in combined length. In another embodiment, the polynucleotide sequences of the first promoter, the second promoter, and the two or more accessory elements for incorporation into the SIRV for packaging into an siAAV have greater than 1381 nucleotides in combined length. In still other embodiments, the polynucleotide sequences of the first promoter, the second promoter, the third promoter, and the two or more accessory elements for incorporation into the SIRV for packaging into an siAAV have greater than at least about 1300, at least about 1350, at least about 1360, at least about 1370, at least about 1380, at least about 1390, at least about 1400, at least about 1500, at least about 1600 nucleotides, at least 1650, at least about 1700, at least about 1750, at least about 1800, at least about 1850, or at least about 1900 nucleotides in combined length. In other embodiments, the polynucleotide sequences of the first promoter, the second promoter, the third promoter, and the two or more accessory elements for incorporation into the SIRV for packaging into an siAAV have greater than at least about 1300 to at least about 1900 nucleotides in combined length. In one embodiment, the polynucleotide sequences of the first promoter, the second promoter, the third promoter, and the two or more accessory elements for incorporation into the SIRV for packaging into an siAAV have greater than 1314 nucleotides in combined length. In another embodiment, the polynucleotide sequences of the first promoter, the second promoter, the third promoter, and the two or more accessory elements for incorporation into the SIRV for packaging into an siAAV have greater than 1381 nucleotides in combined length.

In some embodiments, the present disclosure provides a polynucleotide for use in the SIRV comprising a first adeno-associated virus (AAV) inverted terminal repeat (ITR) sequence, a second AAV ITR sequence, a first promoter sequence, a sequence encoding an SIRV, which comprises a CRISPR protein, a second promoter, a sequence encoding at least a first guide RNA (gRNA), one or more self-inactivating sequences, and one or more accessory element sequences, wherein at least 25%, 26%, 27%, 28%, 29%, 30%, 31%, 32%, 33%, 34%, or 35% or more of the nucleotides of the polynucleotide sequence comprise the first and second promoters, one or more self-inactivating sequences, and the one or more accessory element sequences in combined length. As detailed in the Examples, it has been discovered that the ability to devote more of the total polynucleotide of the expression cassette to the promoters, a second gRNA, and/or the accessory elements results in enhanced expression of and/or performance of the CRISPR protein and gRNA, when expressed in the target host cell; either in an in vitro assay or in vivo in a subject. In some embodiments, the use of alternative or longer promoters and/or accessory elements (e.g., poly(A) signals, NLS, a second gRNA, and/or post-transcriptional regulatory elements) in the SIRV polynucleotides and resulting siAAV vectors results in an increase in editing of a target nucleic acid of at least about 10%, at least about 20%, at least about 30%, at least about 40%, at least about 50%, at least about 60%, at least about 70%, at least about 80%, at least about 90%, at least about 100%, at least about 150%, at least about 200%, or at least about 300% in a timed in vitro assay compared to a construct not having the alternative or longer promoters and/or accessory elements. In one embodiment, the first promoter sequence for incorporation into the SIRV for packaging into an siAAV has at least about 200, at least about 300, at least about 400, at least about 500, at least about 600, at least about 700, or at least about 800 nucleotides. In another embodiment, the second promoter sequence for incorporation into the SIRV for packaging into an siAAV has at least about 200, at least about 300, at least about 400, at least about 500, at least about 600, at least about 700, or at least about 800 nucleotides. In another embodiment, the third promoter sequence for incorporation into the SIRV for packaging into an siAAV has at least about 200, at least about 300, at least about 400, at least about 500, at least about 600, at least about 700, or at least about 800 nucleotides. Representative examples of promoters contemplated for incorporation into the polynucleotide include, but are not limited to the sequences of SEQ ID NOS: 425-431 463-513, and 2688-2708 as set forth in Tables 8, 10, 11, 25, 54, 55, 57, 58. Embodiments of the promoters are described more fully, below.

Due to the smaller size of the CRISPR and regulatory elements utilized in the designs of the SIRV and siAAV, additional components can be incorporated into the transgene to assist in the expression of CRISPR components. In some embodiments, the transgene of the SIRV and siAAV can further comprise one or more accessory elements selected from the group consisting of a poly(A) signal, a gene enhancer element, an intron, a posttranscriptional regulatory element, a nuclear localization signal (NLS), a deaminase, a DNA glycosylase inhibitor, a stimulator of CRISPR-mediated homology-directed repair, and an activator or repressor of transcription. Representative, non-limiting examples of sequences encoding CRISPR proteins (SEQ ID NOS: 747-761, as set forth in Table 63), encoding gRNA (SEQ ID NOS: 462 and 682-710 as set forth in Table 26), promoters (SEQ ID NOS: 425-43, 463-513, and 2688-2708 as set forth in Tables 8, 10, 11, and 25), poly(A) signal sequences (SEQ ID NOS: 514-523 and 2710-2859 as set forth in Tables 12 and 14, and SEQ ID NOS: 2991-3991), PTRE (SEQ ID NOS: 524-526 as set forth in Table 18), enhancers linked to core promoters (SEQ ID NOS: 527-535 as set forth in Table 19), encoded NLS (SEQ ID NOS: 538-587, 599-610, 613, 771-772, 844-846, and 2498-2591 as set forth in Tables 7, 22 and 23), and introns (SEQ ID NOS: 614-658 as set forth in Table 24) suitable for incorporation into the SIRV constructs of the disclosure are presented herein. In some cases, the PTRE is selected from the group consisting of cytomegalovirus immediate/early intronA, hepatitis B virus PRE (HPRE), Woodchuck Hepatitis virus PRE (WPRE), and 5′ untranslated region (UTR) of human heat shock protein 70 mRNA (Hsp70). In some embodiments, the present disclosure provides a polynucleotide for promoters and accessory elements for use in the making of an siAAV vector, wherein the polynucleotide comprises one or more sequences selected from the group of sequences of SEQ ID NOS: 425-431, 463-513-535, 2688-2708, 2710-2859, and 2991-3991, as set forth in Tables 8, 10-12, 14, -18-19, and 25 or a sequence having at least 85%, at least 90%, at least 95%, at least 95%, at least 96%, at least 97%, at least 98%, or at least 99% identity thereto. In another embodiment, the present disclosure provides a polynucleotide for promoters and accessory elements for use in the making of an siAAV vector, wherein the polynucleotide comprises one or more sequence selected from the group of SEQ ID NOS: 425-431, 463-513-535, 2688-2708, 2710-2859, and 2991-3991, set forth in Tables 8, 10-12, 14, 18-19, and 25. It has been discovered that the inclusion of the accessory element(s) in the polynucleotide of the SIRV construct and the transgene of the siAAV can enhance the expression, binding, activity, or performance of the CRISPR protein as compared to the CRISPR protein in the absence of said accessory element in the construct. In one embodiment, the inclusion of the one or more accessory elements in the construct results in an increase in editing of a target nucleic acid by the expressed CRISPR protein in a timed in vitro assay of at least about 10%, at least about 20%, at least about 30%, at least about 40%, at least about 50%, at least about 60%, at least about 70%, at least about 80%, at least about 90%, at least about 100%, at least about 1500%, at least about 200%, or at least about 300% as compared to the CRISPR protein in the absence of said accessory element in the construct.

IV. Guide Nucleic Acids of the SIRV and siAAV Systems

In another aspect, the disclosure relates to guide nucleic acids (gRNA) utilized in the SIRV and siAAV systems that have utility in genome editing or modification of a target nucleic acid in a cell, as well as the inactivation of the constructs. In the case of editing of a target nucleic acid in a cell, the present disclosure provides specifically-designed guide nucleic acids (“gRNAs”) with targeting sequences that are complementary to (and are therefore able to hybridize with) the target nucleic acid as a component of the gene-editing SIRV and siAAV systems, wherein the gRNA is capable of forming a ribonucleoprotein (RNP) complex with a Type V CRISPR nuclease protein, such as a CasX. It is envisioned that in some embodiments, multiple gRNAs are delivered in the SIRV and siAAV systems for the modification of a target nucleic acid. For example, a pair of gRNAs with targeting sequences to different or overlapping regions of the target nucleic acid sequence can be used in order to bind and cleave at two different or overlapping sites within the gene, which is then edited by non-homologous end joining (NHEJ), homology-directed repair (HDR), homology-independent targeted integration (HITI), micro-homology mediated end joining (MMEJ), single strand annealing (SSA) or base excision repair (BER). In the case of inactivation of the SIRV polynucleotide, the present disclosure provides specifically-designed guide nucleic acids (“gRNAs”) with targeting sequences that are complementary to (and are therefore able to hybridize with) the self-inactivating segment(s) in the polynucleotide utilized in the SIRV and siAAV particles, wherein an RNP of the CasX and the gRNA is able to bind and cleave the self-inactivating segment of the double-stranded episome in the target cell.

a. Reference gRNA and gRNA Variants

In some embodiments, the present disclosure provides guide nucleic acids capable of forming a RNP complex with CRISPR nuclease protein for use in the SIRV and siAAV in which the gRNA binds to the CRISPR nuclease protein, and wherein the targeting sequence (or spacer, described more fully, below) of the gRNA is complementary to, and therefore is capable of hybridizing with the target nucleic acid sequence. In some embodiments, the same gRNA is utilized to hybridize with the self-inactivating segment(s). In other embodiments, a second gRNA is incorporated into the polynucleotide construct with an encoded targeting sequence that, when the gRNA is expressed, is complementary to (and are therefore able to hybridize with) the self-inactivating segment(s) of the nucleic acid, leading to cleavage of the self-inactivating segment(s). In some embodiments, the gRNA is a ribonucleic acid molecule (“gRNA”). In some embodiments, the gRNA is a chimera, and comprises both DNA and RNA.

In some embodiments, a gRNA of the present disclosure comprises a sequence of a naturally-occurring gRNA (a “reference gRNA”) that is subjected to one or more mutagenesis methods, such as the mutagenesis methods described herein, which may include Deep Mutational Evolution (DME), deep mutational scanning (DMS), error prone PCR, cassette mutagenesis, random mutagenesis, staggered extension PCR, gene shuffling, or domain swapping, in order to generate one or more gRNA variants with enhanced or varied properties relative to the reference gRNA. The activity of reference gRNAs may be used as a benchmark against which the activity of gRNA variants are compared, thereby measuring improvements in function or other characteristics of the gRNA variants. In other embodiments, a reference gRNA may be subjected to one or more deliberate, targeted mutations in order to produce a gRNA variant, for example a rationally designed variant. As used herein, the term gRNA covers naturally-occurring molecules, as well as sequence variants.

The gRNAs of the disclosure comprise two segments; a targeting sequence and a protein-binding segment. The targeting segment of a gRNA includes a nucleotide sequence (referred to interchangeably as a guide sequence, a spacer, a targeter, or a targeting sequence) that is complementary to (and therefore hybridizes with) a specific sequence (a target site) within the target nucleic acid sequence (e.g., a target ssRNA, a target ssDNA, a strand of a double stranded target DNA, etc.), described more fully below. The targeting sequence of a gRNA is capable of binding to a target nucleic acid sequence, including a coding sequence, a complement of a coding sequence, a non-coding sequence, and to accessory elements. The protein-binding segment (or “activator” or “protein-binding sequence”) interacts with (e.g., binds to) a CasX protein as a complex, forming an RNP (described more fully, below). The protein-binding segment is alternatively referred to herein as a “scaffold”, which is comprised of several regions, described more fully, below.

Once expressed in the cell, a gRNA and a nuclease protein encoded in the SIRV or siAAV systems of the disclosure can form a complex and bind via non-covalent interactions into a complex, e.g., a ribonuclear protein complex (RNP). The gRNA can provide target specificity to the complex by including a targeting sequence having a nucleotide sequence that is complementary to a sequence of a target nucleic acid and/or to the self-inactivating segment. The guide targeting sequence linked 3′ to the scaffold is sometimes referred to herein as the “spacer” or “spacer sequence” or “guide” or “targeting sequence” or “targeting region” of the gRNA. The CRISPR nuclease protein of the complex can provide the site-specific activities of the complex such as cleavage of the target nucleic acid sequence or the self-inactivating segment and/or an activity provided by the fusion partner in the case of a chimeric CRISPR nuclease protein.

Collectively, the assembled gRNAs of the disclosure, including all gRNA variants, comprise distinct structured regions, or domains: the RNA triplex, the scaffold stem loop, the extended stem loop, the pseudoknot, and the targeting sequence that, in the embodiments of the disclosure is specific for a target nucleic acid and is located on the 3′end of the gRNA. The RNA triplex, the scaffold stem loop, the pseudoknot and the extended stem loop, together with the unstructured triplex loop that bridges portions of the triplex, together, are referred to as the “scaffold” of the gRNA. Each of the structured domains are critical to establish the global RNA fold of the guide and retain functionality of the guide; particularly the ability to properly complex with the CasX nuclease. For example, the guide scaffold stem interacts with the helical I domain of CasX nuclease, while residues within the triplex, triplex loop, and pseudoknot stem interact with the OBD of the CasX nuclease. Together, these interactions confer the ability of the guide to bind and form an RNP with the CasX that retains stability, while the spacer (or targeting sequence) directs and defines the specificity of the RNP for binding a specific sequence of DNA. The individual domains are described more fully, below.

b. RNA Triplex and Pseudoknot

In some embodiments of the guide RNAs provided herein (including reference gRNAs) for use in the SIRV and siAAV of the disclosure, there is a RNA triplex, and the RNA triplex comprises the sequence of a UUU--nX(˜4-15)--UUU (SEQ ID NO: 20) stem loop that ends with an AAAG after 2 intervening stem loops (the scaffold stem loop and the extended stem loop), forming a pseudoknot that may also extend past the triplex into a duplex pseudoknot. The UU-UUU-AAA sequence of the triplex forms as a nexus between the targeting sequence, scaffold stem, and extended stem. In exemplary CasX gRNAs, the UUU-loop-UUU region is coded for first, then the scaffold stem loop, and then the extended stem loop, which is linked by the tetraloop, and then an AAAG closes off the triplex before becoming the targeting sequence. The triplex, triplex loop, and pseudoknot stem interact with the OBD of the CasX nuclease. Together, these interactions define RNP binding and stability of the complex.

c. Scaffold Stem Loop

In some embodiments of CasX gRNAs for use in the SIRV and siAAV of the disclosure, the triplex region is followed by the scaffold stem loop. The scaffold stem loop is a region of the gRNA that is bound by CasX protein (such as a CasX variant protein). In some embodiments, the scaffold stem loop is a fairly short and stable stem loop. In some cases, the scaffold stem loop does not tolerate many changes, and requires some form of an RNA bubble. The scaffold stem is necessary for CasX gRNA function as it interacts with the helical I domain of the CasX. While it is perhaps analogous to the nexus stem of Cas9 as being a critical stem loop, the scaffold stem of a CasX gRNA, in some embodiments, has a necessary bulge (RNA bubble) that is different from many other stem loops found in CRISPR/Cas systems. In some embodiments, the presence of this bulge is conserved across gRNA that interact with different CasX proteins.

d. Extended Stem Loop

In some embodiments of the CasX gRNAs for use in the SIRV and siAAV of the disclosure, the scaffold stem loop is followed by the extended stem loop. In some embodiments, the extended stem comprises a synthetic tracr and crRNA fusion that is largely unbound by the CasX protein. In some embodiments, the extended stem loop can be highly malleable. In some embodiments, a single guide gRNA is made with a GAAA tetraloop linker or a GAGAAA linker between the tracr and crRNA in the extended stem loop. In some cases, the targeter and activator of a CasX gRNA are linked to one another by intervening nucleotides and the linker can have a length of from 3 to 20 nucleotides. In some embodiments of the CasX gRNAs of the disclosure, the extended stem is a large 32-bp loop that sits outside of the CasX protein in the ribonucleoprotein complex. In some embodiments, the extended stem loop comprises a GAGAAA linker sequence. In some embodiments, the extended stem loop is modified by insertion of C at position 64 and the A88G substitution relative to the sequence of SEQ ID NO: 2296, which resolves an asymmetrical bulge element of the extended stem, enhancing the stability of the extended stem of the gRNA scaffold.

In some embodiments, the gRNA comprises an extended stem loop region comprising at least 10, at least 100, or at least 500 nucleotides. In some embodiments, the disclosure provides gRNA variants wherein the extended stem loop is modified by inclusion of an RNA stem loop sequence from a heterologous RNA source with proximal 5′ and 3′ ends. In such cases, the heterologous RNA stem loop increases the stability of the gRNA. In some embodiments, the heterologous RNA stem loop is capable of binding a protein, an RNA structure, a DNA sequence, or a small molecule. In some embodiments, an exogenous stem loop region comprises an RNA stem loop or hairpin, for example a thermostable RNA such as MS2 hairpin (SEQ ID NO: 21), Q3 hairpin (SEQ ID NO: 22), U1 hairpin II (SEQ ID NO: 23), Uvsx (SEQ ID NO: 24), PP7 hairpin (SEQ ID NO: 25), Phage replication loop (SEQ ID NO: 26), Kissing loop_a (SEQ ID NO: 27), Kissing loop_b1 (SEQ ID NO: 28), Kissing loop_b2 (SEQ ID NO: 29), G quadriplex M3q (SEQ ID NO: 30), G quadriplex telomere basket (SEQ ID NO: 31), Sarcin-ricin loop (SEQ ID NO: 32), Pseudoknots (SEQ ID NO: 2333), transactivation response element (TAR) (SEQ ID NO: 2333), iron responsive element (IRE) (SEQ ID NO: 2334), phage GA hairpin (SEQ ID NO: 2336), phage AN hairpin (SEQ ID NO: 2337), or sequence variants thereof.

e. Targeting Sequence (a.k.a. Spacer)

In the gRNAs of the disclosure for use in the SIRV and siAAV, the extended stem loop is followed by a region that forms part of the triplex, and then the targeting sequence linked at the 3′ end of the gRNA scaffold. The targeting sequence targets the CasX ribonucleoprotein holo complex to a specific region of the target nucleic acid sequence or the self-inactivating segment. Thus, for example, gRNA targeting sequences of the disclosure have sequences complementarity to, and therefore can hybridize with, a self-inactivating segment and/or to a portion of the target nucleic acid in a eukaryotic cell, (e.g., a eukaryotic chromosome, chromosomal sequence, a eukaryotic RNA, etc.) that is 3′ adjacent to a sequence complementary to a protospacer adjacent motif (PAM) sequence having a TC motif, such as ATC, CTC, GTC, or TTC, in a 5′ to 3′ orientation. In some embodiments, as described more fully, above, the self-inactivating segment comprises the same sequence as the target nucleic acid that is complementary to the targeting sequence of the first gRNA encoded by the SIRV construct. In other embodiments, a second gRNA is encoded by the SIRV wherein the targeting sequence is different from that of the first gRNA and the targeting sequence is complementary to that of the self-inactivating segment.

In the case of gRNA for modification of a target nucleic acid of a cell, the targeting sequence of the first gRNA can be specific for or proximal to a portion of a gene in a eukaryotic cell comprising one or more mutations, wherein modification of the gene is sought. In some embodiments, the targeting sequence of a gRNA is specific for an exon. In some embodiments, the targeting sequence of a gRNA is specific for an intron. In some embodiments, the targeting sequence of the gRNA is specific for an intron-exon junction. In some embodiments, the targeting sequence of a gRNA is specific for an accessory element that regulates expression of a target gene. Such accessory elements include, but are not limited to promoter regions, enhancer regions, intergenic regions, 5′ untranslated regions (5′ UTR), 3′ untranslated regions (3′ UTR), intergenic regions, gene enhancer elements, conserved elements, and regions comprising cis-accessory elements. The promoter region is intended to encompass nucleotides within 5 kb of the target gene initiation point or, in the case of gene enhancer elements or conserved elements, can be 1 Mb or more distal to the target gene. In some embodiments, the targeting sequence of the gRNA is specific for the one or more self-inactivating segments. In some embodiments, the SIRV encodes a first gRNA with a targeting sequence specific for the target nucleic acid and encodes a second gRNA with a targeting sequence specific for the self-inactivating segment. In some embodiments, the targeting sequence of the first and the second gRNA are identical and target both the target nucleic acid and the self-inactivating segment, but cleavage of the self-inactivating segment is modulated by one or more mechanisms described herein; e.g., by use of a weaker PAM adjacent to the self-inactivating segment, by use of a weaker gRNA scaffold, or by introducing mis-matches in 1-3, 1-4 or 1-5 nucleotides in the self-inactivating segment. By selection of the targeting sequences of the gRNA and the overall design of the SIRV construct, defined regions of the target nucleic acid sequence can be modified or edited, and the polynucleotide of the SIRV can be cleaved, using the systems described herein.

In some embodiments, the targeting sequence of the first or the second gRNA has between 14 and 35 consecutive nucleotides. In some embodiments, the targeting sequence has 14, 15, 16, 18, 18, 19, or 20 consecutive nucleotides. In some embodiments, the targeting sequence consists of 21 consecutive nucleotides. In some embodiments, the targeting sequence consists of 20 consecutive nucleotides. In some embodiments, the targeting sequence consists of 19 consecutive nucleotides. In some embodiments, the targeting sequence consists of 18 consecutive nucleotides. In some embodiments, the targeting sequence consists of 17 consecutive nucleotides. In some embodiments, the targeting sequence consists of 16 consecutive nucleotides. In some embodiments, the targeting sequence consists of 15 consecutive nucleotides. In some embodiments, the targeting sequence can comprise 0 to 5, 0 to 4, 0 to 3, or 0 to 2 mismatches relative to the target nucleic acid sequence and retain sufficient binding specificity such that the RNP comprising the gRNA comprising the targeting sequence can form a complementary bond with respect to the target nucleic acid.

f. gRNA Scaffolds

With the exception of the targeting sequence region, the remaining regions of the gRNA are referred to herein as the scaffold. In some embodiments, the gRNA scaffolds are derived from naturally-occurring sequences, described below as reference gRNA. In other embodiments, the gRNA scaffolds are variants of reference gRNA wherein mutations, insertions, deletions or domain substitutions are introduced to confer desirable properties on the gRNA.

In some embodiments, a CasX reference gRNA comprises a sequence isolated or derived from Deltaproteobacter (e.g., SEQ ID NOS: 6, 7 and 34). In some embodiments, a CasX reference guide RNA comprises a sequence isolated or derived from Planctomycetes (e.g., SEQ ID NOS: 8, 9 and 35). In still other embodiments, a CasX reference gRNA comprises a sequence isolated or derived from Candidatus Sungbacteria (e.g., SEQ ID NOS: 10-13).

Table 1 provides the sequences of reference gRNA tracr, cr and scaffold sequences. In some embodiments, the disclosure provides gRNA sequences wherein the gRNA has a scaffold comprising a sequence having at least one nucleotide modification relative to a reference gRNA sequence having a sequence of any one of SEQ ID NOS: 4-16 as set forth in Table 1. It will be understood that in those embodiments wherein a vector comprises a DNA encoding sequence for a gRNA, that thymine (T) bases can be substituted for the uracil (U) bases of any of the gRNA sequence embodiments described herein.

TABLE 1
Reference gRNA tracr and scaffold sequences

| SEQ | |
|---|---|
| ID | |
| NO. | Nucleotide Sequence |
| 4 | ACAUCUGGCGCGUUUAUUCCAUUACUUUGGAGCCAGUCCCAGC |
| | GACUAUGUCGUAUGGACGAAGCGCUUAUUUAUCGGAGAGAAAC |
| | CGAUAAGUAAAACGCAUCAAAG |
| 5 | UACUGGCGCUUUUAUCUCAUUACUUUGAGAGCCAUCACCAGCG |
| | ACUAUGUCGUAUGGGUAAAGCGCUUAUUUAUCGGAGAGAAAUC |
| | CGAUAAAUAAGAAGCAUCAAAG |
| 6 | ACAUCUGGCGCGUUUAUUCCAUUACUUUGGAGCCAGUCCCAGC |
| | GACUAUGUCGUAUGGACGAAGCGCUUAUUUAUCGGAGA |
| 7 | ACAUCUGGCGCGUUUAUUCCAUUACUUUGGAGCCAGUCCCAGC |
| | GACUAUGUCGUAUGGACGAAGCGCUUAUUUAUCGG |
| 8 | UACUGGCGCUUUUAUCUCAUUACUUUGAGAGCCAUCACCAGCG |
| | ACUAUGUCGUAUGGGUAAAGCGCUUAUUUAUCGGAGA |
| 9 | UACUGGCGCUUUUAUCUCAUUACUUUGAGAGCCAUCACCAGCG |
| | ACUAUGUCGUAUGGGUAAAGCGCUUAUUUAUCGG |
| 10 | GUUUACACACUCCCUCUCAUAGGGU |
| 11 | GUUUACACACUCCCUCUCAUGAGGU |
| 12 | UUUUACAUACCCCCUCUCAUGGGAU |
| 13 | GUUUACACACUCCCUCUCAUGGGGG |
| 14 | CCAGCGACUAUGUCGUAUGG |
| 15 | GCGCUUAUUUAUCGGAGAGAAAUCCGAUAAAUAAGAAGC |
| 16 | GGCGCUUUUAUCUCAUUACUUUGAGAGCCAUCACCAGCGACUA |
| | UGUCGUAUGGGUAAAGCGCUUAUUUAUCGGA |

g. gRNA Variants

In another aspect, the disclosure relates to guide nucleic acid variants (referred to herein as “gRNA variant”) for use in the SIRV and siAAV that comprise one or more modifications relative to a reference gRNA scaffold. As used herein, “scaffold” refers to all parts to the gRNA necessary for gRNA function with the exception of the spacer, or targeting sequence.

In some embodiments, a reference gRNA of the disclosure may be subjected to one or more mutagenesis methods, such as the mutagenesis methods described herein (as well as in PCT/US20/36506 and WO2020247883A2, incorporated by reference herein), which may include Deep Mutational Evolution (DME), deep mutational scanning (DMS), error prone PCR, cassette mutagenesis, random mutagenesis, staggered extension PCR, gene shuffling, or domain swapping, in order to generate one or more guide nucleic acid variants (referred to herein as “gRNA variant”) with enhanced or varied properties relative to the reference gRNA. gRNA variants also include variants comprising one or more exogenous sequences, for example fused to either the 5′ or 3′ end, or inserted internally. The activity of reference gRNAs may be used as a benchmark against which the activity of gRNA variants are compared, thereby measuring improvements in function or other characteristics of the gRNA variants. In other embodiments, a reference gRNA may be subjected to one or more deliberate, specifically-targeted mutations in order to produce a gRNA variant, for example a rationally designed variant. In some embodiments, a gRNA variant comprises one or more nucleotide substitutions, insertions, deletions, or swapped or replaced regions relative to a reference gRNA sequence of the disclosure. In some embodiments, a mutation can occur in any region of a reference gRNA scaffold to produce a gRNA variant.

In some embodiments, a gRNA variant comprises one or more nucleotide changes within one or more regions of the reference gRNA scaffold that improve a characteristic of the reference gRNA. A representative example of such a gRNA variant is guide 235 (SEQ ID NO: 2296). Exemplary regions for modification include the RNA triplex, the pseudoknot, the scaffold stem loop, and the extended stem loop. In some cases, the variant scaffold stem further comprises a bubble. In other cases, the variant scaffold further comprises a triplex loop region. In still other cases, the variant scaffold further comprises a 5′ unstructured region. In some embodiments, the gRNA variant scaffold comprises a scaffold stem loop having at least 60% sequence identity, at least 70% sequence identity, at least 80% sequence identity, at least 90% sequence identity, at least 95% sequence identity, or at least 99% sequence identity to SEQ ID NO: 14. In some embodiments, the gRNA variant scaffold comprises a scaffold stem loop having at least 60% sequence identity to SEQ ID NO: 14. In other embodiments, the gRNA variant comprises a scaffold stem loop having the sequence of CCAGCGACUAUGUCGUAGUGG (SEQ ID NO: 36). In other embodiments, the disclosure provides a gRNA scaffold comprising, relative to SEQ ID NO:5, a C18G substitution, a G55 insertion, a U1 deletion, and a modified extended stem loop in which the original 6 nt loop and 13 most-loop-proximal base pairs (32 nucleotides total) are replaced by a Uvsx hairpin (4 nt loop and 5 loop-proximal base pairs; 14 nucleotides total) and the loop-distal base of the extended stem was converted to a fully base-paired stem contiguous with the new Uvsx hairpin by deletion of the A99 and substitution of G65U. In the foregoing embodiment, the gRNA scaffold comprises the sequence

(SEQ ID NO: 2238)
ACUGGCGCUUUUAUCUGAUUACUUUGAGAGCCAUCACCAGCGACUAUGUC
GUAGUGGGUAAAGCUCCCUCUUCGGAGGGAGCAUCAAAG.

All gRNA variants that have one or more improved characteristics, or add one or more new functions, when the variant gRNA is compared to a reference gRNA described herein, are envisaged as within the scope of the disclosure. Exemplary improved characteristics are described in WO2020247882A1 and PCT/US20/36505, incorporated by reference herein. A representative example of such a gNA variant is guide 174 (SEQ ID NO: 2238), the utility of which is described in the Examples. Another representative example of such a gNA variant is guide 235 (SEQ ID NO: 2296), the utility of which is described in the Examples. In some embodiments, the gRNA variant adds a new function to the RNP comprising the gRNA variant. In some embodiments, the gRNA variant has an improved characteristic selected from: improved stability; improved solubility; improved transcription of the gRNA; improved resistance to nuclease activity; increased folding rate of the gRNA; decreased side product formation during folding; increased productive folding; improved binding affinity to a CasX protein; improved binding affinity to a target DNA when complexed with a CasX protein; improved gene editing or modification when complexed with a CasX protein; improved specificity of editing when complexed with a CasX protein, or any combination thereof. In some cases of the foregoing, the improved characteristic is assessed in an in vitro assay, including the assays of the Examples. In other cases of the foregoing, the improved characteristic is assessed in vivo.

In some embodiments, the gRNA variants for use in the SIRV and siAAV systems comprises one or more modifications to the gRNA scaffold variant 174 (SEQ ID NO: 2238) selected from the group consisting of the modifications of Table 47, wherein the resulting gRNA variant exhibits an improved functional characteristic compared to the parent 174, when assessed in an in vitro or in vivo assay under comparable conditions. In some embodiments, the gRNA variants comprising one or more modifications to the gRNA scaffold variant 174 are selected from the group consisting of the modifications of Table 47 (with a linked targeting sequence and complexed with a CasX protein) exhibits an improved enrichment score (log 2) of at least about 2.0, at least about 2.5, at least about 3, or at least about 3.5 greater compared to the score of the gRNA scaffold of SEQ ID NO: 2238 in an in vitro assay, including the assays of the Examples described herein (e.g., Example 28). In a particular embodiment, the one or more modifications of gRNA scaffold variant 174 are selected from the group consisting of nucleotide positions U11, U24, A29, U65, C66, C68, A69, U76, G77, A79, and A87. In a particular embodiment, the modifications of gRNA scaffold variant 174 are U11C, U24C, A29C, U65C, C66G, C68U, an insertion of ACGGA at position 69, an insertion of UCCGU at position 76, G77A, an insertion of GA at position 79, and A87G.

In some embodiments, the gRNA variants for use in the SIRV and siAAV systems comprises one or more modifications to the gRNA scaffold variant 175 (SEQ ID NO: 2239) selected from the group consisting of the modifications of Table 48. In some embodiments, a gRNA variant for use in the SIRV and siAAV systems comprises one or more modifications relative to gRNA scaffold variant 175 (SEQ ID NO: 2239), wherein the resulting gRNA variant exhibits an improved functional characteristic compared to the parent 175, when assessed in an in vitro or in vivo assay under comparable conditions (e.g., the assays of Example 28). For example, variants with modifications to the triplex loop of gRNA variant 175 show high enrichment relative to the 175 scaffold, particularly mutations to C15 or C17. Additionally, changes to either member of the predicted pair in the pseudoknot stem between G7 and A29 are both highly enriched relative to the 175 scaffold, with converting A29 to a C or a T to form a canonical Watson-Crick pairing (G7:C29), and the second of which would form a GU wobble pair (G7:U29), both of which may be expected to increase stability of the helix relative to the G:A pair. In addition, the insertion of a C at position 54 in guide scaffold 175 results in an enriched modification. In some embodiments, the disclosure provides gRNA variants comprising one or more modifications to the gRNA scaffold variant 175 (SEQ ID NO: 2239) are selected from the group consisting of the modifications of Table 48, wherein the resulting gRNA variant exhibits an improved functional characteristic compared to the parent 175, when assessed in an in vitro or in vivo assay under comparable conditions. In some embodiments, the gRNA variants comprising one or more modifications to the gRNA scaffold variant 175 are selected from the group consisting of the modifications of Table 48 (with a linked targeting sequence and complexed with a Class 2, Type V CRISPR protein) exhibits an improved enrichment score (log 2) of at least about 1.2, at least about 1.5, at least about 2.0, at least about 2.5, at least about 3, or at least about 3.5 greater compared to the score of the gRNA scaffold of SEQ ID NO: 2292 in an in vitro assay, including the assays of the Examples described herein. In a particular embodiment, the modifications of gRNA scaffold variant 175 are selected from the group consisting of nucleotide positions C9, U11, C17, U24, A29, G54, C65, A89, and A96. In a particular embodiment, the modifications of gRNA scaffold variant 175 are C9U, U11C, C17G, U24C, A29C, an insertion of G at position 54, an insertion of C at position 65, A89G, and A96G. In one embodiment, the insertion of C at position 64 and the A88G substitution relative to the sequence of SEQ ID NO: 2292 resolves an asymmetrical bulge element of the extended stem, enhancing the stability of the extended stem of the gRNA scaffold. In another embodiment, the substitutions of U11C, U24C, and A95G relative to the sequence of SEQ ID NO: 2292 increases the stability of the triplex region of the gRNA scaffold. In another embodiment, the substitution of A29C relative to the sequence of SEQ ID NO: 2292 increases the stability of the pseudoknot stem. A representative example of such a gRNA variant with improved characteristics relative to gRNA variant from which it was derived is guide 235 (SEQ ID NO: 2296), the utility of which is described in the Examples.

In exemplary embodiments, a gRNA variant for use in the SIRV and siAAV systems comprises one or more modifications relative to gRNA scaffold variant 215 (SEQ ID NO:2276), wherein the resulting gRNA variant exhibits an improved functional characteristic compared to the parent 215, when assessed in an in vitro or in vivo assay under comparable conditions.

In exemplary embodiments, a gRNA variant for use in the SIRV and siAAV systems comprises one or more modifications relative to gRNA scaffold variant 221 (SEQ ID NO: 2282), wherein the resulting gRNA variant exhibits an improved functional characteristic compared to the parent 221, when assessed in an in vitro or in vivo assay under comparable conditions.

In exemplary embodiments, a gRNA variant for use in the SIRV and siAAV systems comprises one or more modifications relative to gRNA scaffold variant 225 (SEQ ID NO: 2286), wherein the resulting gRNA variant exhibits an improved functional characteristic compared to the parent 225, when assessed in an in vitro or in vivo assay under comparable conditions.

In exemplary embodiments, a gRNA variant for use in the SIRV and siAAV systems comprises one or more modifications relative to gRNA scaffold variant 235 (SEQ ID NO: 2296), including CpG depletion, wherein the resulting gRNA variant exhibits an improved functional characteristic compared to the parent 225, when assessed in an in vitro or in vivo assay under comparable conditions.

In exemplary embodiments, a gRNA variant for use in the SIRV and siAAV systems comprises one or more modifications relative to gRNA scaffold variant 251 (SEQ ID NO: 2312), wherein the resulting gRNA variant exhibits an improved functional characteristic compared to the parent 251, when assessed in an in vitro or in vivo assay under comparable conditions.

In exemplary embodiments, a gRNA variant for use in the SIRV and siAAV systems comprises one or more modifications relative to gRNA scaffold variant 316 (SEQ ID NO: 4028), including CpG depletion or chemical modifications, wherein the resulting gRNA variant exhibits an improved functional characteristic compared to the parent 235 and 174, when assessed in an in vitro or in vivo assay under comparable conditions.

In some embodiments, the gRNA variant for use in the SIRV and siAAV systems comprises an exogenous extended stem loop, with such differences from a reference gRNA described as follows. In some embodiments, an exogenous extended stem loop has little or no identity to the reference stem loop regions disclosed herein (e.g., SEQ ID NO: 15). In some embodiments, an exogenous stem loop is at least 10 bp, at least 20 bp, at least 30 bp, at least 40 bp, at least 50 bp, at least 60 bp, at least 70 bp, at least 80 bp, at least 90 bp, at least 100 bp, at least 200 bp, at least 300 bp, at least 400 bp, or at least 500 bp. In some embodiments, the heterologous stem loop increases the stability of the gRNA. In some embodiments, the heterologous RNA stem loop is capable of binding a protein, an RNA structure, a DNA sequence, or a small molecule. In some embodiments, an exogenous stem loop region replacing the stem loop comprises an RNA stem loop or hairpin in which the resulting gRNA has increased stability and, depending on the choice of loop, can interact with certain cellular proteins. Such exogenous extended stem loops can comprise, for example a thermostable RNA such as MS2 hairpin (ACAUGAGGAUCACCCAUGU; SEQ ID NO: 21), Q3 hairpin (AUGCAUGUCUAAGACAGCAU; SEQ ID NO: 22), U1 hairpin II (GGAAUCCAUUGCACUCCGGAUUUCACUAG; SEQ ID NO: 23), Uvsx (CCUCUUCGGAGG; SEQ ID NO: 24), PP7 hairpin (AAGGAGUUUAUAUGGAAACCCUU; SEQ ID NO: 25), Phage replication loop (AGGUGGGACGACCUCUCGGUCGUCCUAUCU; SEQ ID NO: 26), Kissing loop_a (UGCUCGCUCCGUUCGAGCA; SEQ ID NO: 27), Kissing loop_b1 (UGCUCGACGCGUCCUCGAGCA; SEQ ID NO: 28), Kissing loop_b2 (UGCUCGUUUGCGGCUACGAGCA; SEQ ID NO: 29), G quadriplex M3q (AGGGAGGGAGGGAGAGG; SEQ ID NO: 30), G quadriplex telomere basket (GGUUAGGGUUAGGGUUAGG; SEQ ID NO: 31), Sarcin-ricin loop (CUGCUCAGUACGAGAGGAACCGCAG; SEQ ID NO: 32), Pseudoknots (UACACUGGGAUCGCUGAAUUAGAGAUCGGCGUCCUUUCAUUCUAUAUACUUUGGAG UUUUAAAAUGUCUCUAAGUACA; SEQ ID NO: 33), transactivation response element (TAR) (GGCUCGUGUAGCUCAUUAGCUCCGAGCC (SEQ ID NO: 2333)), iron responsive element (IRE) CCGUGUGCAUCCGCAGUGUCGGAUCCACGG (SEQ ID NO: 2334)), phage GA hairpin (AAAACAUAAGGAAAACCUAUGUU (SEQ ID NO: 2336)), phage AN hairpin (GCCCUGAAGAAGGGC (SEQ ID NO: 2337)), or sequence variants thereof. In some embodiments, one of the foregoing hairpin sequences is incorporated into the stem loop of the gRNA scaffold.

Table 2 provides exemplary gRNA variant scaffold sequences of the disclosure. In some embodiments, the gRNA variant scaffold comprises any one of the sequences listed in Table 2, or a sequence having at least about 50%, at least about 60%, at least about 70%, at least about 80%, at least about 90%, at least about 95%, at least about 95%, at least about 96%, at least about 97%, at least about 98%, at least about 99% sequence identity thereto. It will be understood that in those embodiments wherein a vector comprises a DNA encoding sequence for a gRNA, that thymine (T) bases can be substituted for the uracil (U) bases of any of the gRNA sequence embodiments described herein.

TABLE 2
Exemplary gRNA Variant Scaffold Sequences

| SEQ | | |
|---|---|---|
| ID | NAME or | |
| NO: | Modification | NUCLEOTIDE SEQUENCE |
| 2238 | 174 | ACUGGCGCUUUUAUCUGAUUACUUUGAGAGCCAUCACCAGCGACUAU |
| | | GUCGUAGUGGGUAAAGCUCCCUCUUCGGAGGGAGCAUCAAAG |
| 2239 | 175 | ACUGGCGCCUUUAUCUCAUUACUUUGAGAGCCAUCACCAGCGACUAUG |
| | | UCGUAUGGGUAAAGCGCUUACGGACUUCGGUCCGUAAGAAGCAUCAA |
| | | AG |
| 2240 | 176 | GCUGGCGCUUUUAUCUGAUUACUUUGAGAGCCAUCACCAGCGACUAU |
| | | GUCGUAGUGGGUAAAGCUCCCUCUUCGGAGGGAGCAUCAAAG |
| 2241 | 177 | ACUGGCGCUUUUAUCUGAUUACUUUGAGAGCCAUCACCAGCGACUAU |
| | | GUCGUAUGGGUAAAGCUCCCUCUUCGGAGGGAGCAUCAAAG |
| 2242 | 179 | ACUGGCGCUUUUAUCUGAUUACUUUGAGAGCCAUCACCAGCGACUAU |
| | | GUCGUAGUGGGUAAAUAGGAGCUGCACUAUGGGCGCAGUGUCAUUGA |
| | | CGCUGACGGUACAGGCCAGACAAUUAUUGUCUGGUAUAGUGCAGCUC |
| | | CUAAUCAAAG |
| 2243 | 181 | ACUGGCGCCUUUAUCUGAUUACUUUGAGAGCCAUCACCAGCGACUAUG |
| | | UCGUAUGGGUAAAGCGCUUACGGACUUCGGUCCGUAAGAAGCAUCAA |
| | | AG |
| 2244 | 182 | ACUGGCGCUUUUAUCUGAUUACUUUGAGAGCCAUCACCAGCGACUAU |
| | | GUCGUAUGGGUAAAGCGCUUACGGACUUCGGUCCGUAAGAAGCAUCA |
| | | AAG |
| 2245 | 183 | ACUGGCGCUUUUAUCUGAUUACUUUGAGAGCCAUCACCAGCGACUAU |
| | | GUCGUAGUGGGUAAAGCGCUUACGGACUUCGGUCCGUAAGAAGCAUC |
| | | AAAG |
| 2246 | 184 | ACUGGCGCUUUUAUCUGAUUACUUUGAGAGCCAUCACCAGCGACUAU |
| | | GUCGUAUUGGGUAAAGCUCCCUCUUCGGAGGGAGCAUCAAAG |
| 2247 | 185 | ACUGGCGCUUUUAUCUGAUUACUUUGAGAGCCAUCACCAGCGACUAU |
| | | GUCGUAUUGGGUAAAGCGCUUACGGACUUCGGUCCGUAAGAAGCAUC |
| | | AAAG |
| 2248 | 186 | ACUGGCGCCUUUAUCAUCAUUACUUUGAGAGCCAUCACCAGCGACUAU |
| | | GUCGUAUGGGUAAAGCGCUUACGGACUUCGGUCCGUAAGAAGCAUCA |
| | | AAG |
| 2249 | 187 | ACUGGCGCUUUUAUCUGAUUACUUUGAGAGCCAUCACCAGCGACUAU |
| | | GUCGUAGUGGGUAAAGCGCCCUCUUCGGAGGGAAGCAUCAAAG |
| 2250 | 188 | ACUGGCGCUUUUAUCUGAUUACUUUGAGAGCCAUCACCAGCGACUAU |
| | | GUCGUAGUGGGUAAAGCUCACAUGAGGAUCACCCAUGUGAGCAUCAA |
| | | AG |
| 2251 | 189 | ACUGGCACUUUUACCUGAUUACUUUGAGAGCCAACACCAGCGACUAUG |
| | | UCGUAGUGGGUAAAGCUCCCUCUUCGGAGGGAGCAUCAAAG |
| 2252 | 190 | ACUGGCACUUUUAUCUGAUUACUUUGAGAGCCAUCACCAGCGACUAU |
| | | GUCGUAGUGGGUAAAGCUCCCUCUUCGGAGGGAGCAUCAAAG |
| 2253 | 191 | ACUGGCCCUUUUAUCUGAUUACUUUGAGAGCCAUCACCAGCGACUAUG |
| | | UCGUAGUGGGUAAAGCUCCCUCUUCGGAGGGAGCAUCAAAG |
| 2254 | 192 | ACUGGCGCUUUUACCUGAUUACUUUGAGAGCCAUCACCAGCGACUAUG |
| | | UCGUAGUGGGUAAAGCUCCCUCUUCGGAGGGAGCAUCAAAG |
| 2255 | 193 | ACUGGCGCUUUUAUCUGAUUACUUUGAGAGCCAACACCAGCGACUAU |
| | | GUCGUAGUGGGUAAAGCUCCCUCUUCGGAGGGAGCAUCAAAG |
| 2256 | 195 | ACUGGCACCUUUACCUGAUUACUUUGAGAGCCAACACCAGCGACUAUG |
| | | UCGUAUGGGUAAAGCGCUUACGGACUUCGGUCCGUAAGAAGCAUCAA |
| | | AG |
| 2257 | 196 | ACUGGCACCUUUAUCUGAUUACUUUGAGAGCCAUCACCAGCGACUAUG |
| | | UCGUAUGGGUAAAGCGCUUACGGACUUCGGUCCGUAAGAAGCAUCAA |
| | | AG |
| 2258 | 197 | ACUGGCCCCUUUAUCUGAUUACUUUGAGAGCCAUCACCAGCGACUAUG |
| | | UCGUAUGGGUAAAGCGCUUACGGACUUCGGUCCGUAAGAAGCAUCAA |
| | | AG |
| 2259 | 198 | ACUGGCGCCUUUAUCUGAUUACUUUGAGAGCCAACACCAGCGACUAUG |
| | | UCGUAUGGGUAAAGCGCUUACGGACUUCGGUCCGUAAGAAGCAUCAA |
| | | AG |
| 2260 | 199 | GCUGGCGCUUUUAUCUGAUUACUUUGAGAGCCAUCACCAGCGACUAU |
| | | GUCGUAGUGGGUAAAGCUCCCUCUUCGGAGGGAGCAUCAAAG |
| 2261 | 200 | GACUGGCGCUUUUAUCUGAUUACUUUGAGAGCCAUCACCAGCGACUA |
| | | UGUCGUAGUGGGUAAAGCUCCCUCUUCGGAGGGAGCAUCAAAG |
| 2262 | 201 | ACUGGCGCCUUUAUCUGAUUACUUUGGAGAGCCAUCACCAGCGACUAU |
| | | GUCGUAGUGGGUAAAGCUCCCUCUUCGGAGGGAGCAUCAAAG |
| 2263 | 202 | ACUGGCGCAUUUAUCUGAUUACUUUGUGAGCCAUCACCAGCGACUAU |
| | | GUCGUAGUGGGUAAAGCUCCCUCUUCGGAGGGAGCAUCAAAG |
| 2264 | 203 | ACUGGCGCCUUUAUCUGAUUACUUUGAGAGCCAUCACCAGCGACUAUG |
| | | UCGUAGUGGGUAAAGCUCCCUCUUCGGAGGGAGCAUCAAAG |
| 2265 | 204 | ACUGGCGCUUUUAUCUGAUUACUUUGGAGAGCCAUCACCAGCGACUA |
| | | UGUCGUAGUGGGUAAAGCUCCCUCUUCGGAGGGAGCAUCAAAG |
| 2266 | 205 | ACUGGCGCAUUUAUCUGAUUACUUUGAGAGCCAUCACCAGCGACUAU |
| | | GUCGUAGUGGGUAAAGCUCCCUCUUCGGAGGGAGCAUCAAAG |
| 2267 | 206 | ACUGGCGCUUUUAUCUGAUUACUUUGUGAGCCAUCACCAGCGACUAU |
| | | GUCGUAGUGGGUAAAGCUCCCUCUUCGGAGGGAGCAUCAAAG |
| 2268 | 207 | ACUGGCGCUUUUAUUCUGAUUACUUUGAGAGCCAUCACCAGCGACUA |
| | | UGUCGUAGUGGGUAAAGCUCCCUCUUCGGAGGGAGCAUCAAAG |
| 2269 | 208 | ACGGCGCUUUUAUCUGAUUACUUUGAGAGCCAUCACCAGCGACUAUG |
| | | UCGUAGUGGGUAAAGCUCCCUCUUCGGAGGGAGCAUCAAAG |
| 2270 | 209 | ACUGGCGCUUUUAUAUGAUUACUUUGAGAGCCAUCACCAGCGACUAU |
| | | GUCGUAGUGGGUAAAGCUCCCUCUUCGGAGGGAGCAUCAAAG |
| 2271 | 210 | ACUGGCGCUUUUAUCUUGAUUACUUUGAGAGCCAUCACCAGCGACUA |
| | | UGUCGUAGUGGGUAAAGCUCCCUCUUCGGAGGGAGCAUCAAAG |
| 2272 | 211 | ACUGGCGCUUUUAUCUGAUUACUUUGAGAGCCAGCACCAGCGACUAU |
| | | GUCGUAGUGGGUAAAGCUCCCUCUUCGGAGGGAGCAUCAAAG |
| 2273 | 212 | ACUGGCGCUGUUAUCUGAUUACUUCGAGAGCCAUCACCAGCGACUAUG |
| | | UCGUAGUGGGUAAAGCUCCCUCUUCGGAGGGAGCAUCGAAG |
| 2274 | 213 | ACUGGCGCUCUUAUCUGAUUACUUCGAGAGCCAUCACCAGCGACUAUG |
| | | UCGUAGUGGGUAAAGCUCCCUCUUCGGAGGGAGCAUCGAAG |
| 2275 | 214 | ACUGGCGCUUGUAUCUGAUUACUCUGAGAGCCAUCACCAGCGACUAUG |
| | | UCGUAGUGGGUAAAGCUCCCUCUUCGGAGGGAGCAUCAGAG |
| 2276 | 215 | ACUGGCGCUUCUAUCUGAUUACUCUGAGAGCCAUCACCAGCGACUAUG |
| | | UCGUAGUGGGUAAAGCUCCCUCUUCGGAGGGAGCAUCAGAG |
| 2277 | 216 | ACUGGCGCUUUGAUCUGAUUACCUUGAGAGCCAUCACCAGCGACUAUG |
| | | UCGUAGUGGGUAAAGCUCCCUCUUCGGAGGGAGCAUCAAGG |
| 2278 | 217 | ACUGGCGCUUUCAUCUGAUUACCUUGAGAGCCAUCACCAGCGACUAUG |
| | | UCGUAGUGGGUAAAGCUCCCUCUUCGGAGGGAGCAUCAAGG |
| 2279 | 218 | ACUGGCGCUGUUAUCUGAUUACUUUGAGAGCCAUCACCAGCGACUAU |
| | | GUCGUAGUGGGUAAAGCUCCCUCUUCGGAGGGAGCAUCAAAG |
| 2280 | 219 | ACUGGCGCUUUUAUCUGAUUACUUUGAGAGCCAUCACCAGCGACUAU |
| | | GUCGUAGUGGGUAAAGCUCCCUCUUCGGAGGGAGCAUCGAAG |
| 2281 | 220 | ACUGGCGCUUUUAUCUGAUUACUUCGAGAGCCAUCACCAGCGACUAUG |
| | | UCGUAGUGGGUAAAGCUCCCUCUUCGGAGGGAGCAUCAAAG |
| 2282 | 221 | ACUGGCACUUCUAUCUGAUUACUCUGAGAGCCAUCACCAGCGACUAUG |
| | | UCGUAUGGGUAAAGCCGCUUACGGACUUCGGUCCGUAAGAGGCAUCA |
| | | GAG |
| 2283 | 222 | ACUGGCACUUCUAUCUGAUUACUCUGAGAGCCAUCACCAGCGACUAUG |
| | | UCGUAGUGGGUAAAGCUCCCUCUUCGGAGGGAGCAUCAGAG |
| 2284 | 223 | ACUGGCACCUUUAUCUGAUUACUUUGAGAGCCAUCACCAGCGACUAUG |
| | | UCGUAUGGGUAAAGCCGCUUACGGACUUCGGUCCGUAAGAGGCAUCA |
| | | AAG |
| 2285 | 224 | ACUGGCACUUGUAUCUGAUUACUCUGAGAGCCAUCACCAGCGACUAUG |
| | | UCGUAUGGGUAAAGCCGCUUACGGACUUCGGUCCGUAAGAGGCAUCA |
| | | GAG |
| 2286 | 225 | ACUGGCACUUGUAUCUGAUUACUCUGAGAGCCAUCACCAGCGACUAUG |
| | | UCGUAGUGGGUAAAGCUCCCUCUUCGGAGGGAGCAUCAGAG |
| 2287 | 226 | ACUGGCGCUUUUAUCUGAUUACUUUGAGAGCCAUCACCAGCGACUAU |
| | | GUCGUAGUGGGUAAAGCUGCACUAUGGGCGCAGCGUCAAUGACGCUG |
| | | ACGGUACAGGCCAGACAAUUAUUGUCUGGUAUAGUGCAGCAUCAAAG |
| 2288 | 227 | ACUGGCGCUUUUAUCUGAUUACUUUGAGAGCCAUCACCAGCGACUAU |
| | | GUCGUAGUGGGUAAAGCUAGGAGCUUUGUUCCUUGGGUUCUUGGGAG |
| | | CAGCAGGAAGCACUAUGGGCGCAGCGUCAAUGACGCUGACGGUACAG |
| | | GCCAGACAAUUAUUGUCUGGUAUAGUGCAGCAGCAGAACAAUUUGCU |
| | | GAGGGCUAUUGAGGCGCAACAGCAUCUGUUGCAACUCACAGUCUGGG |
| | | GCAUCAAGCAGCUCCAGGCAAGAAUCCUGGCUGUGGAAAGAUACCUA |
| | | AAGGAUCAACAGCUCCUAGCAUCAAAG |
| 2289 | 228 | ACUGGCGCUUUUAUCUGAUUACUUUGAGAGCCAUCACCAGCGACUAU |
| | | GUCGUAGUGGGUAAAGCUCCCCGUACACCAUCAGGGUACGGGGAGCA |
| | | UCAAAG |
| 2290 | 229 | ACUGGCACUUUUAUCUGAUUACUUUGAGAGCCAUCACCAGCGACUAU |
| | | GUCGUAUGGGUAAAGCGCUUACGGACUUCGGUCCGUAAGAAGCAUCA |
| | | AAG |
| 2291 | 230 | ACUGGCACUUCUAUCUGAUUACUCUGAGAGCCAUCACCAGCGACUAUG |
| | | UCGUAUGGGUAAAGCGCUUACGGACUUCGGUCCGUAAGAAGCAUCAG |
| | | AG |
| 2292 | 231 | ACUGGCGCUUCUAUCUGAUUACUCUGAGAGCCAUCACCAGCGACUAUG |
| | | UCGUAUGGGUAAAGCCGCUUACGGACUUCGGUCCGUAAGAGGCAUCA |
| | | GAG |
| 2293 | 232 | ACUGGCACUUCUAUCUGAUUACUCUGAGCGCCAUCACCAGCGACUAUG |
| | | UCGUAUGGGUAAAGCCGCUUACGGACUUCGGUCCGUAAGAGGCAUCA |
| | | GAG |
| 2294 | 233 | ACUGGCGCUUCUAUCUGAUUACUCUGAGCGCCAUCACCAGCGACUAUG |
| | | UCGUAUGGGUAAAGCCGCUUACGGACUUCGGUCCGUAAGAGGCAUCA |
| | | GAG |
| 2295 | 234 | ACUGGCGCUUCUAUCUGAUUACUCUGAGCGCCAUCACCAGCGACUAUG |
| | | UCGUAUGGGUAAAGCGCCUUACGGACUUCGGUCCGUAAGGAGCAUCA |
| | | GAG |
| 2296 | 235 | ACUGGCGCUUCUAUCUGAUUACUCUGAGCGCCAUCACCAGCGACUAUG |
| | | UCGUAGUGGGUAAAGCCGCUUACGGACUUCGGUCCGUAAGAGGCAUC |
| | | AGAG |
| 2297 | 236 | ACGGGACUUUCUAUCUGAUUACUCUGAAGUCCCUCACCAGCGACUAUG |
| | | UCGUAUGGGUAAAGCCGCUUACGGACUUCGGUCCGUAAGAGGCAUCA |
| | | GAG |
| 2298 | 237 | ACCUGUAGUUCUAUCUGAUUACUCUGACUACAGUCACCAGCGACUAUG |
| | | UCGUAUGGGUAAAGCCGCUUACGGACUUCGGUCCGUAAGAGGCAUCA |
| | | GAG |
| 2299 | 238 | ACUGGCGCUUUUAUCUGAUUACUUUGAGAGCCAUCACCAGCGACUAU |
| | | GUCGUAGUGGGUAAAGCUGCACGGUGGGCGCAGCUUCGGCUGACGGU |
| | | ACACCGUGCAGCAUCAAAG |
| 2300 | 239 | ACUGGCGCUUUUAUCUGAUUACUUUGAGAGCCAUCACCAGCGACUAU |
| | | GUCGUAGUGGGUAAAGCUGCACGGUGGGCGCAGCUUCGGCUGACGGU |
| | | ACACCGGUGGGCGCAGCUUCGGCUGACGGUACACCGUGCAGCAUCAAA |
| | | G |
| 2301 | 240 | ACUGGCGCUUUUAUCUGAUUACUUUGAGAGCCAUCACCAGCGACUAU |
| | | GUCGUAGUGGGUAAAGCUGCACGGUGGGCGCAGCUUCGGCUGACGGU |
| | | ACACCGGUGGGCGCAGCUUCGGCUGACGGUACACCGGUGGGCGCAGCU |
| | | UCGGCUGACGGUACACCGUGCAGCAUCAAAG |
| 2302 | 241 | ACUGGCGCUUUUAUCUGAUUACUUUGAGAGCCAUCACCAGCGACUAU |
| | | GUCGUAGUGGGUAAAGCUGCACGGUGGGCGCAGCUUCGGCUGACGGU |
| | | ACACCGGUGGGCGCAGCUUCGGCUGACGGUACACCGGUGGGCGCAGCU |
| | | UCGGCUGACGGUACACCGGUGGGCGCAGCUUCGGCUGACGGUACACCG |
| | | UGCAGCAUCAAAG |
| 2303 | 242 | ACUGGCGCUUUUAUCUGAUUACUUUGAGAGCCAUCACCAGCGACUAU |
| | | GUCGUAGUGGGUAAAGCUGCACGGUGGGCGCAGCUUCGGCUGACGGU |
| | | ACACCGGUGGGCGCAGCUUCGGCUGACGGUACACCGGUGGGCGCAGCU |
| | | UCGGCUGACGGUACACCGGUGGGCGCAGCUUCGGCUGACGGUACACCG |
| | | GUGGGCGCAGCUUCGGCUGACGGUACACCGUGCAGCAUCAAAG |
| 2304 | 243 | ACUGGCGCUUUUAUCUGAUUACUUUGAGAGCCAUCACCAGCGACUAU |
| | | GUCGUAGUGGGUAAAGCUGCACCUAGCGGAGGCUAGGUGCAGCAUCA |
| | | AAG |
| 2305 | 244 | ACUGGCGCUUUUAUCUGAUUACUUUGAGAGCCAUCACCAGCGACUAU |
| | | GUCGUAGUGGGUAAAGCUGCACCUCGGCUUGCUGAAGCGCGCACGGCA |
| | | AGAGGCGAGGUGCAGCAUCAAAG |
| 2306 | 245 | ACUGGCGCUUUUAUCUGAUUACUUUGAGAGCCAUCACCAGCGACUAU |
| | | GUCGUAGUGGGUAAAGCUGCACCUCUCUCGACGCAGGACUCGGCUUGC |
| | | UGAAGCGCGCACGGCAAGAGGCGAGGGGCGGCGACUGGUGAGUACGC |
| | | CAAAAAUUUUGACUAGCGGAGGCUAGAAGGAGAGAGGUGCAGCAUCA |
| | | AAG |
| 2307 | 246 | ACUGGCGCUUUUAUCUGAUUACUUUGAGAGCCAUCACCAGCGACUAU |
| | | GUCGUAGUGGGUAAAGCUGCACGGUGCCCGUCUGUUGUGUCGAGAGA |
| | | CGCCAAAAAUUUUGACUAGCGGAGGCUAGAAGGAGAGAGAUGGGUGC |
| | | CGUGCAGCAUCAAAG |
| 2308 | 247 | ACUGGCGCUUUUAUCUGAUUACUUUGAGAGCCAUCACCAGCGACUAU |
| | | GUCGUAGUGGGUAAAGCUGCACAUGGAGAGGAGAUGUGCAGCAUCAA |
| | | AG |
| 2309 | 248 | ACUGGCGCUUUUAUCUGAUUACUUUGAGAGCCAUCACCAGCGACUAU |
| | | GUCGUAGUGGGUAAAGCUGCACAUGGAGAUGUGCAGCAUCAAAG |
| 2310 | 249 | ACUGGCGCUUUUAUCUGAUUACUUUGAGAGCCAUCACCAGCGACUAU |
| | | GUCGUAGUGGGUAAAGCUUGGGCGCAGCGUCAAUGACGCUGACGGUA |
| | | CAAGCAUCAAAG |
| 2311 | 250 | ACUGGCGCUUUUAUCUGAUUACUUUGAGAGCCAUCACCAGCGACUAU |
| | | GUCGUAGUGGGUAAAGCUGCACUAUGGGCGCAGCGUCAAUGACGCUG |
| | | ACGGUACAGGCCACAUGAGGAUCACCCAUGUGGUAUAGUGCAGCAUC |
| | | AAAG |
| 2312 | 251 | ACUGGCGCUUUUAUCUGAUUACUUUGAGAGCCAUCACCAGCGACUAU |
| | | GUCGUAGUGGGUAAAGCUGCACUAUGGGCGCAGCUCAUGAGGAUCAC |
| | | CCAUGAGCUGACGGUACAGGCCACAUGAGGAUCACCCAUGUGGUAUA |
| | | GUGCAGCAUCAAAG |
| 2313 | 252 | ACUGGCGCUUUUAUCUGAUUACUUUGAGAGCCAUCACCAGCGACUAU |
| | | GUCGUAGUGGGUAAAGCUGCACUAUGGGCGCAGCGUCAAUGACGCUG |
| | | ACGGUACAGGCCACAUGGCAGUCGUAACGACGCGGGUGGUAUAGUGC |
| | | AGCAUCAAAG |
| 2314 | 253 | ACUGGCGCUUUUAUCUGAUUACUUUGAGAGCCAUCACCAGCGACUAU |
| | | GUCGUAGUGGGUAAAGCUGCACUAUGGGCGCAGCAAACAUGGCAGUC |
| | | CUAAGGACGCGGGUUUUGCUGACGGUACAGGCCACAUGGCAGUCGUA |
| | | ACGACGCGGGUGGUAUAGUGCAGCAUCAAAG |
| 2315 | 254 | ACUGGCGCUUUUAUCUGAUUACUUUGAGAGCCAUCACCAGCGACUAU |
| | | GUCGUAGUGGGUAAAGCUGCACUAUGGGCGCAGACAUGGCAGUCGUA |
| | | ACGACGCGGGUCUGACGGUACAGGCCACAUGAGGAUCACCCAUGUGGU |
| | | AUAGUGCAGCAUCAAAG |
| 2316 | 255 | ACUGGCGCUUUUAUCUGAUUACUUUGAGAGCCAUCACCAGCGACUAU |
| | | GUCGUAGUGGGUAAAGCUGCACUAAGGAGUUUAUAUGGAAACCCUUA |
| | | GUGCAGCAUCAAAG |
| 2317 | 256 | ACUGGCGCUUUUAUCUGAUUACUUUGAGAGCCAUCACCAGCGACUAU |
| | | GUCGUAGUGGGUAAAGCUCAGGAAGCACUAUGGGCGCAGCGUCAAUG |
| | | ACGCUGACGGUACAGGCCAGACAAUUAUUGUCUGGUAUAGUGCAGCA |
| | | GCAGAACAAUUUGCUGAGGGCUAUUGAGGCGCAACAGCAUCUGUUGC |
| | | AACUCACAGUCUGGGGCAUCAAGCAGCUCCAGGCAAGAAUCCUGAGCA |
| | | UCAAAG |
| 2318 | 257 | ACUGGCGCUUUUAUCUGAUUACUUUGAGAGCCAUCACCAGCGACUAU |
| | | GUCGUAGUGGGUAAAGCUGCACGCCCUGAAGAAGGGCGUGCAGCAUC |
| | | AAAG |
| 2319 | 258 | ACUGGCGCUUUUAUCUGAUUACUUUGAGAGCCAUCACCAGCGACUAU |
| | | GUCGUAGUGGGUAAAGCUGCACGGCUCGUGUAGCUCAUUAGCUCCGA |
| | | GCCGUGCAGCAUCAAAG |
| 2320 | 259 | ACUGGCGCUUUUAUCUGAUUACUUUGAGAGCCAUCACCAGCGACUAU |
| | | GUCGUAGUGGGUAAAGCUGCACCCGUGUGCAUCCGCAGUGUCGGAUCC |
| | | ACGGGUGCAGCAUCAAAG |
| 2321 | 260 | ACUGGCGCUUUUAUCUGAUUACUUUGAGAGCCAUCACCAGCGACUAU |
| | | GUCGUAGUGGGUAAAGCUGCACGGAAUCCAUUGCACUCCGGAUUUCA |
| | | CUAGGUGCAGCAUCAAAG |
| 2322 | 261 | ACUGGCGCUUUUAUCUGAUUACUUUGAGAGCCAUCACCAGCGACUAU |
| | | GUCGUAGUGGGUAAAGCUGCACAUGCAUGUCUAAGACAGCAUGUGCA |
| | | GCAUCAAAG |
| 2323 | 262 | ACUGGCGCUUUUAUCUGAUUACUUUGAGAGCCAUCACCAGCGACUAU |
| | | GUCGUAGUGGGUAAAGCUGCACAAAACAUAAGGAAAACCUAUGUUGU |
| | | GCAGCAUCAAAG |
| 2324 | 263 | ACUGGCGCUUCUAUCUGAUUACUCUGAGCGCCAUCACCAGCGACUAUG |
| | | UCGUAGUGGGUAAAGCCGCUUACGGACUAUGGGCGCAGCGUCAAUGA |
| | | CGCUGACGGUACAGGCCAGACAAUUAUUGUCUGGUAUAGUCCGUAAG |
| | | AGGCAUCAGAG |
| 2325 | 264 | ACUGGCGCUUCUAUCUGAUUACUCUGAGCGCCAUCACCAGCGACUAUG |
| | | UCGUAGUGGGUAAAGCCGCUUACGGGUGGGCGCAGCGUCAAUGACGC |
| | | UGACGGUACAGGCCAGACAAUUAUUGUCUGGUACCCGUAAGAGGCAU |
| | | CAGAG |
| 2326 | 265 | ACUGGCGCUUCUAUCUGAUUACUCUGAGCGCCAUCACCAGCGACUAUG |
| | | UCGUAGUGGGUAAAGCCGCUUACGGACUAUGGGCGCAGCGUCAAUGA |
| | | CGCUGACGGUACAGGCCACAUGAGGAUCACCCAUGUGGUAUAGUCCGU |
| | | AAGAGGCAUCAGAG |
| 2327 | 266 | ACUGGCGCUUUUAUCUGAUUACUUUGAGAGCCAUCACCAGCGACUAU |
| | | GUCGUAGUGGGUAAAGCUCCCUAUGGGCGCAGCGUCAAUGACGCUGA |
| | | CGGUACAGGCCACAUGAGGAUCACCCAUGUGGUAUAGGGAGCAUCAA |
| | | AG |
| 2328 | 267 | ACUGGCGCUUCUAUCUGAUUACUCUGAGCGCCAUCACCAGCGACUAUG |
| | | UCGUAGUGGGUAAAGCCGCUUACGGACUAUGGGCGCAGCUCAUGAGG |
| | | AUCACCCAUGAGCUGACGGUACAGGCCACAUGAGGAUCACCCAUGUGG |
| | | UAUAGUCCGUAAGAGGCAUCAGAG |
| 2329 | 268 | ACUGGCGCUUUUAUCUGAUUACUUUGAGAGCCAUCACCAGCGACUAU |
| | | GUCGUAGUGGGUAAAGCUCCCUAUGGGCGCAGCUCAUGAGGAUCACCC |
| | | AUGAGCUGACGGUACAGGCCACAUGAGGAUCACCCAUGUGGUAUAGG |
| | | GAGCAUCAAAG |
| 2330 | 269 | ACUGGCGCUUCUAUCUGAUUACUCUGAGCGCCAUCACCAGCGACUAUG |
| | | UCGUAGUGGGUAAAGCCGCUUACGGACUAUGGGCGCAGCGUCAAUGA |
| | | CGCUGACGGUACAGGCCACAUGGCAGUCGUAACGACGCGGGUGGUAU |
| | | AGUCCGUAAGAGGCAUCAGAG |
| 2331 | 270 | ACUGGCGCUUUUAUCUGAUUACUUUGAGAGCCAUCACCAGCGACUAU |
| | | GUCGUAGUGGGUAAAGCUCCCUAUGGGCGCAGCGUCAAUGACGCUGA |
| | | CGGUACAGGCCACAUGGCAGUCGUAACGACGCGGGUGGUAUAGGGAG |
| | | CAUCAAAG |
| 3992 | 271 | ACUGGCGCUUCUAUCUGAUUACUCUGAGCGCCAUCACCAGCGACUAUG |
| | | UCGUAGUGGGUAAAGCCGCUUACGGACUAUGGGCGCAGCAAACAUGG |
| | | CAGUCCUAAGGACGCGGGUUUUGCUGACGGUACAGGCCACAUGGCAG |
| | | UCGUAACGACGCGGGUGGUAUAGUCCGUAAGAGGCAUCAGAG |
| 3993 | 272 | ACUGGCGCUUUUAUCUGAUUACUUUGAGAGCCAUCACCAGCGACUAU |
| | | GUCGUAGUGGGUAAAGCUCCCUAUGGGCGCAGCAAACAUGGCAGUCC |
| | | UAAGGACGCGGGUUUUGCUGACGGUACAGGCCACAUGGCAGUCGUAA |
| | | CGACGCGGGUGGUAUAGGGAGCAUCAAAG |
| 3994 | 273 | ACUGGCGCUUCUAUCUGAUUACUCUGAGCGCCAUCACCAGCGACUAUG |
| | | UCGUAGUGGGUAAAGCCGCUUACGGACUAUGGGCGCAGACAUGGCAG |
| | | UCGUAACGACGCGGGUCUGACGGUACAGGCCACAUGAGGAUCACCCAU |
| | | GUGGUAUAGUCCGUAAGAGGCAUCAGAG |
| 3995 | 274 | ACUGGCGCUUUUAUCUGAUUACUUUGAGAGCCAUCACCAGCGACUAU |
| | | GUCGUAGUGGGUAAAGCUCCCUAUGGGCGCAGACAUGGCAGUCGUAA |
| | | CGACGCGGGUCUGACGGUACAGGCCACAUGAGGAUCACCCAUGUGGUA |
| | | UAGGGAGCAUCAAAG |
| 4028 | 316 | ACTGGCGCTTCTATCTGATTACTCTGAGCGCCATCACCAGCGACTATGTC |
| | | GTAGTGGGTAAAGCTCCCTCTTCGGAGGGAGCATCAGAG |

In some embodiments, the scaffold of the gRNA for use in the siAAV comprises SEQ ID NOS: 2101-2237.

In some embodiments, the scaffold of the gRNA variant(s) encoded by the polynucleotide of the SIRV comprises an exogenous extended stem loop, with such differences from a reference gRNA described as follows. In some embodiments, an exogenous extended stem loop has little or no identity to the reference stem loop regions disclosed herein (e.g., SEQ ID NO: 15). In some embodiments, an exogenous stem loop is at least 10 bp, at least 20 bp, at least 30 bp, at least 40 bp, at least 50 bp, at least 60 bp, at least 70 bp, at least 80 bp, at least 90 bp, at least 100 bp, at least 200 bp, at least 300 bp, at least 400 bp, or at least 500 bp. In some embodiments, the 5′ and 3′ ends of the exogenous stem loop are base paired; i.e., interact to form a region of duplex RNA. In some embodiments, the 5′ and 3′ ends of the exogenous stem loop are base paired, and one or more regions between the 5′ and 3′ ends of the exogenous stem loop are not base paired. In some embodiments, the at least one nucleotide modification comprises: (a) substitution of 1 to 15 consecutive or non-consecutive nucleotides in the gRNA variant in one or more regions; (b) a deletion of 1 to 10 consecutive or non-consecutive nucleotides in the gRNA variant in one or more regions; (c) an insertion of 1 to 10 consecutive or non-consecutive nucleotides in the gRNA variant in one or more regions; (d) a substitution of the scaffold stem loop or the extended stem loop with an RNA stem loop sequence from a heterologous RNA source with proximal 5′ and 3′ ends; or any combination of (a)-(d).

In some embodiments, the scaffold of the gRNA variant(s) encoded by the polynucleotide of the SIRV comprises a sequence or subsequence of any one of SEQ ID NOS: 2238, 2239, 2240, 2242, 2246, 2250, 2251, 2261-2287, 2291, 2296, or 4028 and a sequence of an exogenous stem loop.

In some embodiments, the scaffold of the gRNA variant(s) encoded by the polynucleotide of the SIRV comprises a scaffold stem loop having at least 60% identity to SEQ ID NO: 14. In some embodiments, the gRNA variant comprises a scaffold stem loop having at least 60% identity, at least 70% identity, at least 80% identity, at least 90% identity, at least 95% identity, at least 98% identity or at least 99% identity to SEQ ID NO: 14. In some embodiments, the gRNA variant comprises a scaffold stem loop comprising SEQ ID NO: 14.

In some embodiments, the scaffold of the gRNA variant(s) encoded by the polynucleotide of the SIRV comprises a scaffold stem loop sequence of CCAGCGACUAUGUCGUAGUGG (SEQ ID NO: 36). In some embodiments, the gRNA variant comprises a scaffold stem loop sequence of CCAGCGACUAUGUCGUAGUGG (SEQ ID NO: 36) with at least 1, 2, 3, 4, or 5 mismatches thereto.

In some embodiments, the scaffold of the gRNA variant(s) encoded by the polynucleotide of the SIRV comprises one or more modifications relative to the sequence of another gRNA variant.

In some embodiments, the scaffold of the gRNA variant(s) encoded by the polynucleotide of the SIRV comprises a sequence of SEQ ID NO:2104, SEQ ID NO:2106, SEQ ID NO:2163, SEQ ID NO:2107, SEQ ID NO:2164, SEQ ID NO:2165, SEQ ID NO:2166, SEQ ID NO:2103, SEQ ID NO:2167, SEQ ID NO:2105, SEQ ID NO:2108, SEQ ID NO:2112, SEQ ID NO:2160, SEQ ID NO:2170, SEQ ID NO:2114, SEQ ID NO:2171, SEQ ID NO:2112, SEQ ID NO:2173, SEQ ID NO:2102, SEQ ID NO:2174, SEQ ID NO:2175, SEQ ID NO:2109, SEQ ID NO:2176, SEQ ID NO:2238, SEQ ID NO:2239, SEQ ID NO:2240, SEQ ID NO:2241, SEQ ID NO:2274, SEQ ID NO:2276, SEQ ID NO: 2279, SEQ ID NO: 2286, SEQ ID NO: 2289, SEQ ID NO: 2296, or SEQ ID NO: 4028.

In some embodiments, the scaffold of the gRNA variant(s) encoded by the polynucleotide of the SIRV comprises one or more modifications relative to the sequence of another gRNA variant. In some embodiments, the gRNA variant comprises one or more additional changes to a sequence of any one of SEQ ID NOs: 2201-2286. In some embodiments, the gRNA variant comprises a sequence of any one of SEQ ID NOS: 2238, 2239, 2240, 2243, 2246, 2250, 2251, 2261-2286, 2289, 2296, or 4028, or a sequence having at least about 70%, at least about 80%, at least about 90%, at least about 95%, at least about 96%, at least about 97%, at least about 98%, at least about 99% identity thereto.

In some embodiments, the scaffold of the gRNA variant(s) encoded by the polynucleotide of the SIRV comprises the sequence of any one of SEQ ID NOS: 2201-2286, 2289, 2296, or 4028 of Table 2. In some embodiments, the scaffold of the gRNA consists or consists essentially of the sequence of any one of SEQ ID NOS: 2201-2286, 2289 2296, or 4028. In some embodiments, the scaffold of the gRNA variant sequence is at least about 60% identical, at least about 65% identical, at least about 70% identical, at least about 75% identical, at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical or at least about 99% identical to any one of SEQ ID NOS: 2201-2286, 2289, 2296, or 4028. In exemplary embodiments the gRNA variant retains the ability to bind a CasX. In a particular embodiment, the gRNA variant comprises a sequence of any one of SEQ ID NOS: 2238, 2239, or 2296.

In some embodiments, the encoded gRNA variant of the SIRV further comprises a spacer (or targeting sequence) region located at the 3′ end of the gRNA, described more fully, supra, which comprises at least 14 to about 20 nucleotides wherein the spacer is designed with a sequence that is complementary to a target DNA. In some embodiments, the targeting sequence has 14, 15, 16, 17, 18, 19, or 20 nucleotides. In some embodiments, the encoded gRNA variant comprises a targeting sequence having 20 nucleotides. In some embodiments, the targeting sequence has 19 nucleotides. In some embodiments, the targeting sequence has 18 nucleotides. In some embodiments, the targeting sequence has 17 nucleotides. In some embodiments, the targeting sequence has 16 nucleotides. In some embodiments, the targeting sequence has 15 nucleotides. In some embodiments, the targeting sequence has 14 nucleotides.

V. CRISPR Proteins of the SIRV and siAAV Systems

The present disclosure provides SIRV and siAAV systems encoding a CRISPR nuclease that have utility in genome editing or modification of eukaryotic cells, as well as being an integral component of the self-inactivating feature of the construct. In some embodiments, the CRISPR nuclease employed in the genome-editing systems is a Class 2, Type V nuclease. Although members of Class 2, Type V CRISPR-Cas systems have differences, they share some common characteristics that distinguish them from the Cas9 systems. Firstly, the Class 2, Type V nucleases possess a single RNA-guided RuvC domain-containing effector but no HNH domain, and they recognize T-rich PAM 5′ upstream to the target region on the non-targeted strand, which is different from Cas9 systems which rely on G-rich PAM at 3′ side of target sequences. Type V nucleases generate staggered double-stranded breaks distal to the PAM sequence, unlike Cas9, which generates a blunt end in the proximal site close to the PAM. In addition, Type V nucleases degrade ssDNA in trans when activated by target dsDNA or ssDNA binding in cis. In some embodiments, the expressed Type V nucleases of the SIRV and siAAV embodiments recognize a 5′-TC PAM motif and produce staggered ends cleaved solely by the RuvC domain. In some embodiments, the Type V nuclease is selected from the group consisting of Cas12a (Cpf1), Cas12b (C2c1), Cas12c (C2c3), Cas12d (CasY), Cas12e (CasX), Cas12f, Cas12g, Cas12h, Cas12i, Cas12j, Cas12k, Cas14, and Cas(D. In some embodiments, the Type V nuclease for incorporation in the SIRV and siAAV of the disclosure has an encoding DNA sequence of less than about 2950 nucleotides, less than about 2940 nucleotides, less than about 2900 nucleotides, less than about 2850 nucleotides, less than about 2800 nucleotides, less than about 2750 nucleotides, less than about 2700 nucleotides, less than about 2650 nucleotides, less than about 2600 nucleotides, less than about 2550 nucleotides, less than about 2450 nucleotides, or less than about 2450 nucleotides.

In some embodiments, the present disclosure provides SIRV and siAAV systems encoding a Class 2 Type V protein, e.g., a CasX protein and one or more gRNA acids that upon expression in a cell are able to form an RNP complex and are specifically designed to modify a target nucleic acid sequence in eukaryotic cells, as well as cleave the self-inactivating segments utilized in the polynucleotide comprising the transgene of the SIRV construct.

The term “CasX protein”, as used herein, refers to a family of proteins, and encompasses all naturally occurring CasX proteins, proteins that share at least 50% identity to naturally occurring CasX proteins, as well as CasX variants possessing one or more improved characteristics relative to a naturally-occurring CasX protein, described more fully, below. CasX proteins of the disclosure comprise at least the following domains: a non-target strand binding (NTSB) domain, a target strand loading (TSL) domain, a helical I domain, a helical II domain, an oligonucleotide binding domain (OBD), and a RuvC DNA cleavage domain, or a subdomain thereof, as listed in Tables 3 and 4.

A CasX protein functions as an endonuclease that catalyzes a double strand break at a specific sequence in a targeted double-stranded DNA (dsDNA). In some embodiments, the encoded CasX of the system is a reference CasX. In other embodiments, the CasX protein is not a naturally-occurring protein (e.g., the CasX protein is a CasX variant protein, a chimeric protein, and the like).

The editing specificity of the CasX:gRNA RNP is provided by the targeting sequence of the associated gRNA, which hybridizes to a sequence within the target nucleic acid sequence or the self-inactivating segment, as described supra.

In some embodiments, a CasX protein can bind and/or modify (e.g., cleave, nick, methylate, demethylate, etc.) a target nucleic acid sequence and/or a polypeptide associated with the target nucleic acid sequence (e.g., methylation or acetylation of a histone tail).

a. Reference CasX Proteins

The disclosure provides wild-type reference CasX proteins and the polynucleotides that encode them for use in the SIRV and siAAV systems. In some embodiments, the reference CasX proteins are modified to create CasX variants for use in the SIRV and siAAV systems. In some embodiments, a reference CasX protein is derived from a naturally-occurring protein. For example, reference CasX proteins can be isolated or cloned from naturally occurring prokaryotes, such as Deltaproteobacter, Planctomycetes, or Candidatus Sungbacteria species. A reference CasX protein (interchangeably referred to herein as a reference CasX polypeptide) is a type II CRISPR/Cas endonuclease belonging to the CasX (interchangeably referred to as Cas12e) family of proteins that interacts with a guide RNA to form a ribonucleoprotein (RNP) complex.

In some cases, a reference CasX protein is isolated or derived from Deltaproteobacter having a sequence of:

| (SEQ ID NO: 1) | |
|---|---|
| 1 | MEKRINKIRK KLSADNATKP VSRSGPMKTL LVRVMTDDLK KRLEKRRKKP EVMPQVISNN |
| 61 | AANNLRMLLD DYTKMKEAIL QVYWQEFKDD HVGLMCKFAQ PASKKIDQNK LKPEMDEKGN |
| 121 | LTTAGFACSQ CGQPLFVYKL EQVSEKGKAY TNYFGRCNVA EHEKLILLAQ LKPEKDSDEA |
| 181 | VTYSLGKFGQ RALDFYSIHV TKESTHPVKP LAQIAGNRYA SGPVGKALSD ACMGTIASFL |
| 241 | SKYQDIIIEH QKVVKGNQKR LESLRELAGK ENLEYPSVTL PPQPHTKEGV DAYNEVIARV |
| 301 | RMWVNLNLWQ KLKLSRDDAK PLLRLKGFPS FPVVERRENE VDWWNTINEV KKLIDAKRDM |
| 361 | GRVFWSGVTA EKRNTILEGY NYLPNENDHK KREGSLENPK KPAKRQFGDL LLYLEKKYAG |
| 421 | DWGKVFDEAW ERIDKKIAGL TSHIEREEAR NAEDAQSKAV LTDWLRAKAS FVLERLKEMD |
| 481 | EKEFYACEIQ LQKWYGDLRG NPFAVEAENR VVDISGFSIG SDGHSIQYRN LLAWKYLENG |
| 541 | KREFYLLMNY GKKGRIRFTD GTDIKKSGKW QGLLYGGGKA KVIDLTFDPD DEQLIILPLA |
| 601 | FGTRQGREFI WNDLLSLETG LIKLANGRVI EKTIYNKKIG RDEPALFVAL TFERREVVDP |
| 661 | SNIKPVNLIG VDRGENIPAV IALTDPEGCP LPEFKDSSGG PTDILRIGEG YKEKQRAIQA |
| 721 | AKEVEQRRAG GYSRKFASKS RNLADDMVRN SARDLFYHAV THDAVLVFEN LSRGFGRQGK |
| 781 | RTFMTERQYT KMEDWLTAKL AYEGLTSKTY LSKTLAQYTS KTCSNCGFTI TTADYDGMLV |
| 841 | RLKKTSDGWA TTLNNKELKA EGQITYYNRY KRQTVEKELS AELDRLSEES GNNDISKWTK |
| 901 | GRRDEALFLL KKRFSHRPVQ EQFVCLDCGH EVHADEQAAL NIARSWLFLN SNSTEFKSYK |
| 961 | SGKQPFVGAW QAFYKRRLKE VWKPNA. |

In some cases, a reference CasX protein is isolated or derived from Planctomycetes having a sequence of:

| (SEQ ID NO: 2) | |
|---|---|
| 1 | MQEIKRINKI RRRLVKDSNT KKAGKTGPMK TLLVRVMTPD LRERLENLRK KPENIPQPIS |
| 61 | NTSRANLNKL LTDYTEMKKA ILHVYWEEFQ KDPVGLMSRV AQPAPKNIDQ RKLIPVKDGN |
| 121 | ERLTSSGFAC SQCCQPLYVY KLEQVNDKGK PHTNYFGRCN VSEHERLILL SPHKPEANDE |
| 181 | LVTYSLGKFG QRALDFYSIH VTRESNHPVK PLEQIGGNSC ASGPVGKALS DACMGAVASF |
| 241 | LTKYQDIILE HQKVIKKNEK RLANLKDIAS ANGLAFPKIT LPPQPHTKEG IEAYNNVVAQ |
| 301 | IVIWVNLNLW QKLKIGRDEA KPLQRLKGFP SFPLVERQAN EVDWWDMVCN VKKLINEKKE |
| 361 | DGKVFWQNLA GYKRQEALLP YLSSEEDRKK GKKFARYQFG DLLLHLEKKH GEDWGKVYDE |
| 421 | AWERIDKKVE GLSKHIKLEE ERRSEDAQSK AALTDWLRAK ASFVIEGLKE ADKDEFCRCE |
| 481 | LKLQKWYGDL RGKPFAIEAE NSILDISGFS KQYNCAFIWQ KDGVKKLNLY LIINYFKGGK |
| 541 | LRFKKIKPEA FEANRFYTVI NKKSGEIVPM EVNFNFDDPN LIILPLAFGK RQGREFIWND |
| 601 | LLSLETGSLK LANGRVIEKT LYNRRTRQDE PALFVALTFE RREVLDSSNI KPMNLIGIDR |
| 661 | GENIPAVIAL TDPEGCPLSR FKDSLGNPTH ILRIGESYKE KQRTIQAAKE VEQRRAGGYS |
| 721 | RKYASKAKNL ADDMVRNTAR DLLYYAVTQD AMLIFENLSR GFGRQGKRTF MAERQYTRME |
| 781 | DWLTAKLAYE GLPSKTYLSK TLAQYTSKTC SNCGFTITSA DYDRVLEKLK KTATGWMTTI |
| 841 | NGKELKVEGQ ITYYNRYKRQ NVVKDLSVEL DRLSEESVNN DISSWTKGRS GEALSLLKKR |
| 901 | FSHRPVQEKF VCLNCGFETH ADEQAALNIA RSWLFLRSQE YKKYQTNKTT GNTDKRAFVE |
| 961 | TWQSFYRKKL KEVWKPAV. |

In some cases, a reference CasX protein is isolated or derived from Candidatus Sungbacteria having a sequence of

| (SEQID NO: 3 | |
|---|---|
| 1 | MDNANKPSTK SLVNITRISD HFGVTPGQVT RVFSFGIIPT KRQYAIIERW FAAVEAARER |
| 61 | LYGMLYAHFQ ENPPAYLKEK FSYETFFKGR PVLNGLRDID PTIMTSAVFT ALRHKAEGAM |
| 121 | AAFHTNHRRL FEEARKKMRE YAECLKANEA LLRGAADIDW DKIVNALRTR LNTCLAPEYD |
| 181 | AVIADFGALC AFRALIAETN ALKGAYNHAL NQMLPALVKV DEPEEAEESP RLRFFNGRIN |
| 241 | DLPKFPVAER ETPPDTETII RQLEDMARVI PDTAEILGYI HRIRHKAARR KPGSAVPLPQ |
| 301 | RVALYCAIRM ERNPEEDPST VAGHFLGEID RVCEKRRQGL VRTPFDSQIR ARYMDIISFR |
| 361 | ATLAHPDRWT EIQFLRSNAA SRRVRAETIS APFEGFSWTS NRTNPAPQYG MALAKDANAP |
| 421 | ADAPELCICL SPSSAAFSVR EKGGDLIYMR PTGGRRGKDN PGKEITWVPG SFDEYPASGV |
| 481 | ALKLRLYFGR SQARRMLTNK TWGLLSDNPR VFAANAELVG KKRNPQDRWK LFFHMVISGP |
| 541 | PPVEYLDFSS DVRSRARTVI GINRGEVNPL AYAVVSVEDG QVLEEGLLGK KEYIDQLIET |
| 601 | RRRISEYQSR EQTPPRDLRQ RVRHLQDTVL GSARAKIHSL IAFWKGILAI ERLDDQFHGR |
| 661 | EQKIIPKKTY LANKTGFMNA LSFSGAVRVD KKGNPWGGMI EIYPGGISRT CTQCGTVWLA |
| 721 | RRPKNPGHRD AMVVIPDIVD DAAATGFDNV DCDAGTVDYG ELFTLSREWV RLTPRYSRVM |
| 781 | RGTLGDLERA IRQGDDRKSR QMLELALEPQ PQWGQFFCHR CGFNGQSDVL AATNLARRAI |
| 841 | SLIRRLPDTD TPPTP. |

b. Class 2 Type V: CasX Variant Proteins

The present disclosure provides variants of a reference CasX protein (interchangeably referred to herein as “CasX variant” or “CasX variant protein”) for use in the SIRV and siAAV systems, wherein the CasX variants comprise one or more modifications in at least one domain relative to the reference CasX protein, including the sequences of SEQ ID NOS:1-3, or one or more modifications relative to another CasX variant from which it was derived; e.g. CasX 491 (SEQ ID NO: 138) or CasX 515 (SEQ ID NO: 145). Any change in amino acid sequence of a reference CasX protein that leads to an improved characteristic of the CasX protein is considered a CasX variant protein of the disclosure. For example, CasX variants can comprise one or more amino acid substitutions, insertions, deletions, or swapped domains, or any combinations thereof, relative to a reference CasX protein sequence. Any permutation of the substitution, insertion and deletion embodiments described herein can be combined to generate a CasX variant protein of the disclosure.

Exemplary improved characteristics of the CasX variant embodiments include, but are not limited to improved folding of the variant, improved binding affinity to the gRNA, improved binding affinity to the target nucleic acid, improved ability to utilize a greater spectrum of PAM sequences in the editing and/or binding of target DNA, improved unwinding of the target DNA, increased editing activity, improved editing efficiency, improved editing specificity, increased percentage of a eukaryotic genome that can be efficiently edited, increased activity of the nuclease, increased target strand loading for double strand cleavage, decreased target strand loading for single strand nicking, decreased off-target cleavage, improved binding of the non-target strand of DNA, improved protein stability, improved protein:gRNA (RNP) complex stability, improved protein solubility, improved protein:gRNA (RNP) complex solubility, improved protein yield, improved protein expression, and improved fusion characteristics, as described more fully, below. Exemplary improved characteristics are described in WO2020247882A1 and PCT/US20/36505, incorporated by reference herein. In the foregoing embodiments, the one or more of the improved characteristics of the CasX variant is at least about 1.1 to about 100,000-fold improved relative to the reference CasX protein of SEQ ID NO: 1, SEQ ID NO: 2, or SEQ ID NO: 3, when assayed in a comparable fashion. In other embodiments, the improvement is at least about 1.1-fold, at least about 2-fold, at least about 5-fold, at least about 10-fold, at least about 50-fold, at least about 100-fold, at least about 500-fold, at least about 1000-fold, at least about 5000-fold, at least about 10,000-fold, or at least about 100,000-fold compared to the reference CasX protein of SEQ ID NO: 1, SEQ ID NO: 2, or SEQ ID NO: 3, or CasX 491 (SEQ ID NO: 138) or CasX 515 (SEQ ID NO: 145) when assayed in a comparable fashion. In other embodiments, the improvement is at least about 1.1-fold, at least about 2-fold, at least about 5-fold, at least about 10-fold, at least about 50-fold, at least about 100-fold, at least about 500-fold, at least about 1000-fold, at least about 5000-fold, at least about 10,000-fold, or at least about 100,000-fold compared to the reference CasX protein of SEQ ID NO: 1, SEQ ID NO: 2, or SEQ ID NO: 3, or CasX 491 (SEQ ID NO: 138) or CasX 515 (SEQ ID NO: 145) when assayed in a comparable fashion. In other cases, the one or more improved characteristics of an RNP of the CasX variant and the gRNA variant are at least about 1.1, at least about 10, at least about 100, at least about 1000, at least about 10,000, at least about 100,000-fold or more improved relative to an RNP of the reference CasX protein of SEQ ID NO:1, SEQ ID NO:2, or SEQ ID NO:3 and the reference gRNA of SEQ ID NOS: 4-16 of Table 1 or the RNP of CasX 491 (SEQ ID NO: 138) or CasX 515 (SEQ ID NO: 145) and gRNA variants of SEQ ID NOS: of Table 2, optionally with gRNA 174 (SEQ ID NO: 2238). In other cases, the one or more of the improved characteristics of an RNP of the CasX variant and the gRNA variant are about 1.1 to 100,00-fold, about 1.1 to 10,00-fold, about 1.1 to 1,000-fold, about 1.1 to 500-fold, about 1.1 to 100-fold, about 1.1 to 50-fold, about 1.1 to 20-fold improved relative to an RNP of the reference CasX protein of SEQ ID NO:1, SEQ ID NO:2, or SEQ ID NO:3 and the reference gRNA of SEQ ID NOS: 4-16 of Table 1 or the RNP of CasX 491 (SEQ ID NO: 138) or CasX 515 (SEQ ID NO: 145) and gRNA variants of SEQ ID NOS: of Table 2, optionally with gRNA 174 (SEQ ID NO: 2238), when assayed in a comparable fashion.

An exemplary improved characteristic includes improved editing efficiency, wherein an RNP of a CasX variant and a gRNA variant exhibit an improved cleavage rate of a target nucleic acid of at least 2-fold, at least 3-fold, at least 4-fold, at least 5-fold, at lease 6-fold, at least 7-fold, at least 8-fold, or at least 10-fold or greater compared to an RNP of a reference wild-type CasX and reference gRNA, when assayed in vitro under comparable conditions, as demonstrated in the Examples, below. In some embodiments of the SIRV and siAAV, upon expression and the forming of the RNP complex, the RNP of a CasX variant and a gRNA variant at a concentration of 20 μM or less, is capable of cleaving a double stranded DNA target with an efficiency of at least 40%, at least 50%, at least 60%, at least 70%, at least 80%, at least 85%, at least 90% or at least 95%. In some embodiments, the RNP of a CasX variant and a gRNA variant at a concentration of 50 μM or less, 40 μM or less, 30 μM or less, 20 μM or less, 10 μM or less, or 5 μM or less, is capable of cleaving a double stranded DNA target with an efficiency of at least 40%, at least 50%, at least 60%, at least 70%, at least 80%, at least 85%, at least 90% or at least 95%, greatly exceeding the performance of RNP of an RNP of a reference wild-type CasX and reference gRNA. The improved editing efficiency of the CasX variants, in combination with the gRNA variants of the disclosure, make them well-suited for inclusion in the SIRV and siAAV of the disclosure compared to a reference wild-type CasX and reference gRNA.

In some embodiments, the modification of the CasX variant is a mutation in one or more amino acids of the reference CasX. In other embodiments, the modification is an insertion or substitution of a part or all of a domain from a different CasX protein. In a particular embodiment, the CasX variants of 514-791 have a NTSB and helical Ib domain of SEQ ID NO: 1, while the other domains are derived from SEQ ID NO: 2, in addition to individual modifications in select domains, described herein and, thus, the CasX variants are chimeric. In some embodiment, the disclosure provides CasX variants for use in the SIRV and siAAV wherein the CasX comprises a RuvC cleavage domain, wherein the RuvC cleavage domain comprises the sequence of amino acids 648-812 of SEQ ID NO: 2 with one or more amino acid modifications relative to said RuvC cleavage domain sequence. In some embodiments, the one or more amino acid modifications of the RuvC domain comprise a modification at a position selected from the group consisting of 1658, A708, and P793. Mutations can be introduced in any one or more domains of the reference CasX protein or in a CasX variant to result in a CasX variant, and may include, for example, deletion of part or all of one or more domains, or one or more amino acid substitutions, deletions, or insertions in any domain of the reference CasX protein or the CasX variant from which it was derived.

In some embodiments, the CasX variant protein comprises at least one modification in at least 1 domain, in at least each of 2 domains, in at least each of 3 domains, in at least each of 4 domains or in at least each of 5 domains of the reference CasX protein, including the sequences of SEQ ID NOS: 1-3, or a CasX variant from which it was derived.

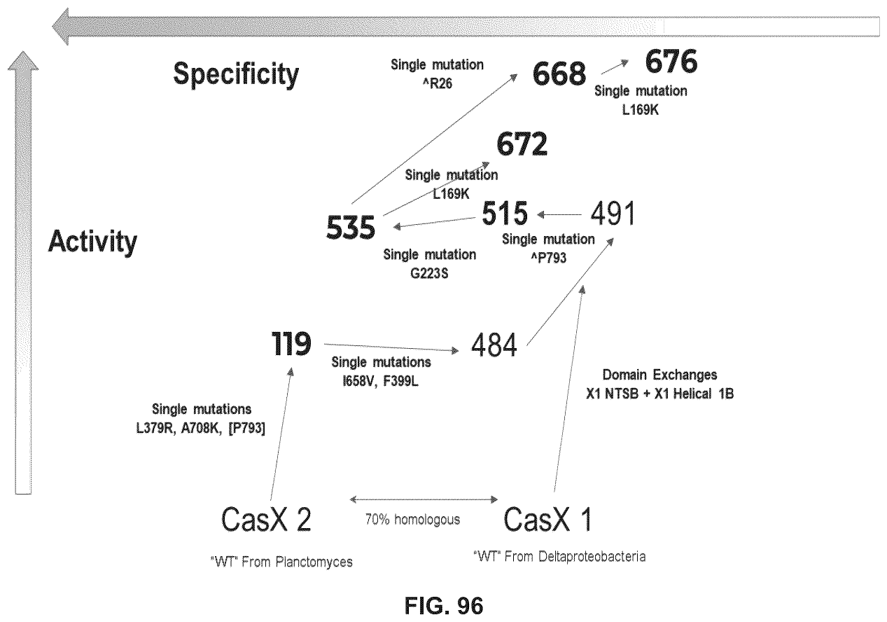

In other embodiments, the disclosure provides CasX variants for use in the SIRV and siAAV wherein the CasX variants comprise at least one modification relative to another CasX variant; e.g., CasX variant 515 and 527 is a variant of CasX variant 491 and CasX variants 668 and 672 are variants of CasX 535 (see, FIG. 96). In some embodiments, the at least one modification is selected from the group consisting of an amino acid insertion, deletion, or substitution. All variants that improve one or more functions or characteristics of the CasX variant protein when compared to a reference CasX protein or the variant from which it was derived described herein are envisaged as being within the scope of the disclosure. A CasX variant can be mutagenized to create another CasX variant. In a particular embodiment, the disclosure provides variants of CasX 515 created by introducing modifications to the encoding sequence resulting in amino acid substitutions, deletions, or insertions at one or more positions in one or more domains or subdomains of CasX 515.

Suitable mutagenesis methods for generating CasX variant proteins of the disclosure may include, for example, Deep Mutational Evolution (DME), deep mutational scanning (DMS), error prone PCR, cassette mutagenesis, random mutagenesis, staggered extension PCR, gene shuffling, or domain swapping (described in PCT/US20/36506 and WO2020247883A2, incorporated by reference herein). In some embodiments, the CasX variants are designed, for example by selecting multiple desired mutations in a CasX variant identified using assays described in the Examples. In certain embodiments, the activity of a reference CasX or the CasX variant protein prior to mutagenesis is used as a benchmark against which the activity of one or more resulting CasX variants are compared, thereby measuring improvements in function of the new CasX variants.

The CasX variants of the embodiments described herein have the ability to form an RNP complex with the gRNA variants disclosed herein. The CasX variant proteins of the disclosure have an enhanced ability to efficiently edit and/or bind target DNA, when complexed with a gRNA variant as an RNP, utilizing a PAM TC motif, including PAM sequences selected from TTC, ATC, GTC, or CTC, compared to an RNP of a reference CasX protein and reference gRNA. In the foregoing, the PAM sequence is located at least 1 nucleotide 5′ to the non-target strand of the protospacer having identity with the targeting sequence of the gRNA variant in an assay system compared to the editing efficiency and/or binding of an RNP comprising a reference CasX protein and reference gRNA in a comparable assay system. In one embodiment, an RNP of a CasX variant and gRNA variant exhibits greater editing efficiency and/or binding of a target sequence in the target DNA compared to an RNP comprising a reference CasX protein and a reference gRNA in a comparable assay system, wherein the PAM sequence of the target DNA is TTC. In another embodiment, an RNP of a CasX variant and gRNA variant exhibits greater editing efficiency and/or binding of a target sequence in the target DNA compared to an RNP comprising a reference CasX protein and a reference gRNA in a comparable assay system, wherein the PAM sequence of the target DNA is ATC. In another embodiment, an RNP of a CasX variant and gRNA variant exhibits greater editing efficiency and/or binding of a target sequence in the target DNA compared to an RNP comprising a reference CasX protein and a reference gRNA in a comparable assay system, wherein the PAM sequence of the target DNA is CTC. In another embodiment, an RNP of a CasX variant and gRNA variant exhibits greater editing efficiency and/or binding of a target sequence in the target DNA compared to an RNP comprising a reference CasX protein and a reference gRNA in a comparable assay system, wherein the PAM sequence of the target DNA is GTC. In the foregoing embodiments, the increased editing efficiency and/or binding affinity for the one or more PAM sequences is at least 1.5-fold greater or more compared to the editing efficiency and/or binding affinity of an RNP of any one of the CasX proteins of SEQ ID NOS:1-3 and the gRNA of Table 1 for the PAM sequences.

The term “CasX variant” is inclusive of variants that are fusion proteins; i.e., the CasX is “fused to” a heterologous sequence. This includes CasX variants comprising CasX variant sequences and N-terminal, C-terminal, or internal fusions of the CasX to a heterologous protein or domain thereof.

In some embodiments, the CasX variant protein comprises between 400 and 2000 amino acids, between 500 and 1500 amino acids, between 700 and 1200 amino acids, between 800 and 1100 amino acids or between 900 and 1000 amino acids.

c. CasX Variant Proteins with Domains from Multiple Source Proteins

In certain embodiments, the disclosure provides a chimeric CasX protein for use in the SIRV and siAAV systems comprising protein domains from two or more different CasX proteins, such as two or more reference CasX proteins, or two or more CasX variant protein sequences as described herein, or a reference CasX protein and a CasX variant protein. As used herein, a “chimeric CasX protein” refers to a CasX containing at least two domains isolated or derived from different sources, such as two naturally occurring proteins, which may, in some embodiments, be isolated from different species. In a particular embodiment, the CasX variants of 514-791 have a NTSB and helical 1b domain derived from the sequence of SEQ ID NO: 1, while the other domains are derived from SEQ ID NO: 2, it being understood that the variants have additional amino acid changes at select locations. In another particular, embodiment, the CasX variant of 494 has a NTSB domain derived from the sequence of SEQ ID NO: 1, while the other domains are derived from SEQ ID NO: 2.

In some embodiments of the SIRV and siAAV systems, a CasX variant protein comprises at least one chimeric domain comprising a first part from a first CasX protein and a second part from a second, different CasX protein. As used herein, a “chimeric domain” refers to a domain containing at least two parts isolated or derived from different sources, such as two naturally occurring proteins or portions of domains from two reference CasX proteins, the domain coordinates of which are provided in Table 3 and the sequences of which are provided in Table 4. The at least one chimeric domain can be any of the NTSB, TSL, helical I, helical II, OBD or RuvC domains as described herein. As an example of the foregoing, the chimeric RuvC domain comprises amino acids 661 to 824 of SEQ ID NO: 1 and amino acids 922 to 978 of SEQ ID NO: 2. As an alternative example of the foregoing, a chimeric RuvC domain comprises amino acids 648 to 812 of SEQ ID NO: 2 and amino acids 935 to 986 of SEQ ID NO: 1. In the case of split or non-contiguous domains such as helical I, RuvC and OBD, a portion of the non-contiguous domain can be replaced with the corresponding portion from any other source. For example, the helical I-I domain (sometimes referred to as helical I-a) in SEQ ID NO: 2 can be replaced with the corresponding helical I-I sequence from SEQ ID NO: 1, and the like. Domain sequences from reference CasX proteins, and their coordinates, are shown in Tables 3 and 4. Representative examples of chimeric CasX proteins include the variants of CasX 472-483, 485-491 and 515, the sequences of which are set forth in Table 5.

TABLE 3
Domain coordinates in Reference CasX proteins

| | Coordinates in | Coordinates in |
|---|---|---|
| Domain Name | SEQ ID NO: 1 | SEQ ID NO: 2 |
| OBD a | 1-55 | 1-57 |
| helical I a | 56-99 | 58-101 |
| NTSB | 100-190 | 102-191 |
| helical I b | 191-331 | 192-332 |
| helical II | 332-508 | 333-500 |
| OBD b | 509-659 | 501-646 |
| RuvC a | 660-823 | 647-810 |
| TSL | 824-933 | 811-920 |
| RuvC b | 934-986 | 921-978 |

*OBD a and b, helical I a and b, and RuvC a and b are also referred to herein as OBD I and II, helical I-I and I-II, and RuvC I and II.

Exemplary domain sequences are provided in Table 4 below.

TABLE 4
Exemplary Domain Sequences in Reference
CasX proteins

| | | |
|---|---|---|
| SEQ | | |
| ID | | |
| NO | Domain | Sequence |

Deltaproteobacter sp. (reference CasX of
SEQ ID NO: 1)

| | | |
|---|---|---|
| 2338 | OBD-I | EKRINKIRKKLSADNATKPVSRSGPMKTLLVRVM |
| | | TDDLKKRLEKRRKKPEVMPQ |
| 2339 | helical | VISNNAANNLRMLLDDYTKMKEAILQVYWQEFKD |
| | I-I | DHVGLMCKFA |
| 2340 | NTSB | QPASKKIDQNKLKPEMDEKGNLTTAGFACSQCGQ |
| | | PLFVYKLEQVSEKGKAYTNYFGRCNVAEHEKLIL |
| | | LAQLKPEKDSDEAVTYSLGKFGQ |
| 2341 | helical | RALDFYSIHVTKESTHPVKPLAQIAGNRYASGPV |
| | I-II | GKALSDACMGTIASFLSKYQDIIIEHQKVVKGNQ |
| | | KRLESLRELAGKENLEYPSVTLPPQPHTKEGVDA |
| | | YNEVIARVRMWVNLNLWQ KLKLSRDDAKPLLRL |
| | | KGFPSF |
| 2342 | helical | PVVERRENEVDWWNTINEVKKLIDAKRDMGRVFW |
| | II | SGVTAEKRNTILEGYNYLPNENDHKKREGSLENP |
| | | KKPAKRQFGDLLLYLEKKYAGDWGKVFDEAWERI |
| | | DKKIAGLTSHIEREEARNAEDAQSKAVLTDWLRA |
| | | KASFVLERLKEMDEKEFYACEIQLQKWYGDLRG |
| | | NPFAVEAE |
| 2343 | OBD-II | NRVVDISGFSIGSDGHSIQYRNLLAWKYLENGKR |
| | | EFYLLMNYGKKGRIRFTDGTDIKKSGKWQGLLYG |
| | | GGKAKVIDLTFDPDDEQLIILPLAFGTRQGREFI |
| | | WNDLLSLETGLIKLANGRVIEKTIYNKKIG RDE |
| | | PALFVALTFERREVVD |
| 2344 | RuvC-I | PSNIKPVNLIGVDRGENIPAVIALTDPEGCPLPE |
| | | FKDSSGGPTDILRIGEGYKEKQRAIQAAKEVEQR |
| | | RAGGYSRKFASKSRNLADDMVRNSARDLFYHAVT |
| | | HDAVLVFENLSRGFGRQGKRTFMTERQYTKMEDW |
| | | LTAKLAYEGLTSKTYLSKTLAQYTSKTC |
| 2345 | TSL | SNCGFTITTADYDGMLVRLKKTSDGWATTLNNKE |
| | | LKAEGQITYYNRYKRQTVEKELSAELDRLSEESG |
| | | NNDISKWTKGRRDEALFLLKKRFSHRPVQEQFVC |
| | | LDCGHEVH |
| 2346 | RuvC-II | ADEQAALNIARSWLFLN SNSTEFKSYKSGKQPF |
| | | VGAWQAFYKRRLKEVWKPNA |

Planctomycetes sp. (Reference CasX of
SEQ ID NO: 2)

| | | |
|---|---|---|
| 2347 | OBD-I | QEIKRINKIRRRLVKDSNTKKAGKTGPMKTLLVR |
| | | VMTPDLRERLENLRKKPENIPQ |
| 2348 | helical | PISNTSRANLNKLLTDYTEMKKAILHVYWEEFQK |
| | I-II | DPVGLMSRVA |
| 2349 | NTSB | QPAPKNIDQRKLIPVKDGNERLTSSGFACSQCCQ |
| | | PLYVYKLEQVNDKGKPHTNYFGRCNVSEHERLIL |
| | | LSPHKPEANDELVTYSLGKFGQ |
| 2350 | helical | RALDFYSIHVTRESNHPVKPLEQIGGNSCASGPV |
| | I-II | GKALSDACMGAVASFLTKYQDIILEHQKVIKKNE |
| | | KRLANLKDIASANGLAFPKITLPPQPHTKEGIEA |
| | | YNNVVAQIVIWVNLNLWQKLKIGRDEAKPLQRLK |
| | | GFPSF |
| 2351 | helical | PLVERQANEVDWWDMVCNVKKLINEKKEDGKVFW |
| | II | QNLAGYKRQEALLPYLSSEEDRKKGKKFARYQFG |
| | | DLLLHLEKKHGEDWGKVYDEAWERIDKKVEGLSK |
| | | HIKLEEERRSEDAQSKAALTDWLRAKASFVIEGL |
| | | KEADKDEFCRCELKLQKWYGDLRGKPFAIEAE |
| 2352 | OBD-II | NSILDISGFSKQYNCAFIWQKDGVKKLNLYLIIN |
| | | YFKGGKLRFKKIKPEAFEANRFYTVINKKSGEIV |
| | | PMEVNFNFDDPNLIILPLAFGKRQGREFIWNDLL |
| | | SLETGSLKLANGRVIEKTLYNRRTRQDEPALFVA |
| | | LTFERREVLD |
| 2353 | RuvC-I | SSNIKPMNLIGIDRGENIPAVIALTDPEGCPLSR |
| | | FKDSLGNPTHILRIGESYKEKQRTIQAAKEVEQR |
| | | RAGGYSRKYASKAKNLADDMVRNTARDLLYYAVT |
| | | QDAMLIFENLSRGFGRQGKRTFMAERQYTRMED |
| | | LTAKLAYEGLPSKTYLSKTLAQYTSKTCW |
| 2354 | TSL | SNCGFTITSADYDRVLEKLKKTATGWMTTINGKE |
| | | LKVEGQITYYNRYKRQNVVKDLSVELDRLSEESV |
| | | NNDISSWTKGRSGEALSLLKKRFSHRPVQEKFVC |
| | | LNCGFETH |
| 2355 | RuvC- | ADEQAALNIARSWLFLRSQEYKKYQTNKTTGNTD |
| | II | KRAFVETWQSFYRKKLKEVWKPAV |

d. Exemplary CasX Variants

In some embodiments, a Class 2 Type V, CasX variant protein for use in the SIRV and siAAV systems comprises a sequence of SEQ ID NOS: 49-321 and 2356-2488, or a sequence as set forth in Table 5. In some embodiments, a CasX variant protein for use in the SIRV and siAAV systems comprises a sequence set forth in Table 5, including the sequences of SEQ ID NOS: 72-321 and 2356-2488. In some embodiments, a CasX variant protein consists of a sequence selected from the group consisting of SEQ ID NOS: 72-321 and 2356-2488. In other embodiments, a Class 2 Type V, CasX variant protein comprises a sequence at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical to a sequence selected from the group consisting of SEQ ID NOS: 49-321 and 2356-2488. In a particular embodiment, a CasX variant protein for use in the SIRV and siAAV systems comprises the sequence of SEQ ID NO: 138, or a sequence having at least about 70%, at least about 80%, at least about 80%, at least about 85%, at least about 90%, or at least about 95%, or at least about 95%, or at least about 96%, or at least about 97%, or at least about 98%, or at least about 99% sequence identity thereto. In another particular embodiment, a CasX variant protein for use in the SIRV and siAAV systems comprises the sequence of SEQ ID NO: 145, or a sequence having at least about 70%, at least about 80%, at least about 80%, at least about 85%, at least about 90%, or at least about 95%, or at least about 95%, or at least about 96%, or at least about 97%, or at least about 98%, or at least about 99% sequence identity thereto. In a particular embodiment, a CasX variant protein for use in the SIRV and siAAV systems comprises the sequence of SEQ ID NO: 303, or a sequence having at least about 70%, at least about 80%, at least about 80%, at least about 85%, at least about 90%, or at least about 95%, or at least about 95%, or at least about 96%, or at least about 97%, or at least about 98%, or at least about 99% sequence identity thereto. In exemplary embodiments, the CasX retain nuclease activity and the ability to form an RNP with a gRNA. It will be understood that in most cases, upon expression, the CasX variant will not have the N-terminal methionine due to post-translational modification.

TABLE 5
CasX Variant Sequences

| SEQ | |
|---|---|
| ID NO: | Description |
| 72 | 119 |
| 73 | substitution of L379R, a substitution of C477K, a substitution of |
| | A708K, a deletion of P at position 793 and a substitution of |
| | M771N of SEQ ID NO: 2. |
| 74 | substitution of A708K, a deletion of P at position 793 and a |
| | substitution of E386S of SEQ ID NO: 2. |
| 75 | substitution of L379R, a substitution of C477K, a substitution of |
| | A708K and a deletion of P at position 793 of SEQ ID NO: 2. |
| 76 | substitution of L792D of SEQ ID NO: 2. |
| 77 | substitution of G791F of SEQ ID NO: 2. |
| 78 | substitution of A708K, a deletion of P at position 793 and a |
| | substitution of A739V of SEQ ID NO: 2. |
| 79 | substitution of L379R, a substitution of A708K, a deletion of P |
| | at position 793 and a substitution of A739V of SEQ ID NO: 2. |
| 80 | substitution of C477K, a substitution of A708K and a deletion of |
| | P at position 793 of SEQ ID NO: 2. |
| 81 | substitution of L249I and a substitution of M771N of SEQ ID |
| | NO: 2. |
| 82 | substitution of V747K of SEQ ID NO: 2. |
| 83 | substitution of L379R, a substitution of C477K, a substitution of |
| | A708K, a deletion of P at position 793 and a substitution of |
| | M779N of SEQ ID NO: 2. |
| 84 | substitution of L379R, substitution of F755M of SEQ ID NO: 2. |
| 85 | 429 |
| 86 | 430 |
| 87 | 431 |
| 88 | 432 |
| 89 | 433 |
| 90 | 434 |
| 91 | 435 |
| 92 | 436 |
| 93 | 437 |
| 94 | 438 |
| 95 | 439 |
| 96 | 440 |
| 97 | 441 |
| 98 | 442 |
| 99 | 443 |
| 100 | 444 |
| 101 | 445 |
| 102 | 446 |
| 103 | 447 |
| 104 | 448 |
| 105 | 449 |
| 106 | 450 |
| 107 | 451 |
| 108 | 452 |
| 109 | 453 |
| 110 | 454 |
| 111 | 455 |
| 112 | 456 |
| 113 | 457 |
| 114 | 458 |
| 115 | 459 |
| 116 | 460 |
| 117 | 278 |
| 118 | 279 |
| 119 | 280 |
| 120 | 285 |
| 121 | 286 |
| 122 | 287 |
| 123 | 288 |
| 124 | 290 |
| 125 | 291 |
| 126 | 293 |
| 127 | 300 |
| 128 | 492 |
| 129 | 493 |
| 130 | 387 |
| 131 | 395 |
| 132 | 485 |
| 133 | 486 |
| 134 | 487 |
| 135 | 488 |
| 136 | 489 |
| 137 | 490 |
| 138 | 491 |
| 139 | 494 |
| 140 | 328 |
| 141 | 388 |
| 142 | 389 |
| 143 | 390 |
| 144 | 514 |
| 145 | 515 |
| 146 | 516 |
| 147 | 517 |
| 148 | 518 |
| 149 | 519 |
| 150 | 520 |
| 151 | 522 |
| 152 | 523 |
| 153 | 524 |
| 154 | 525 |
| 155 | 526 |
| 156 | 527 |
| 157 | 528 |
| 158 | 529 |
| 159 | 530 |
| 160 | 531 |
| 161 | 532 |
| 162 | 533 |
| 163 | 534 |
| 164 | 535 |
| 165 | 536 |
| 166 | 537 |
| 167 | 538 |
| 168 | 539 |
| 169 | 540 |
| 170 | 541 |
| 171 | 542 |
| 172 | 543 |
| 173 | 544 |
| 174 | 545 |
| 175 | 546 |
| 176 | 547 |
| 177 | 548 |
| 178 | 550 |
| 179 | 551 |
| 180 | 552 |
| 181 | 553 |
| 182 | 554 |
| 183 | 555 |
| 184 | 556 |
| 185 | 557 |
| 186 | 558 |
| 187 | 559 |
| 188 | 560 |
| 189 | 561 |
| 190 | 562 |
| 191 | 563 |
| 192 | 564 |
| 193 | 565 |
| 194 | 566 |
| 195 | 567 |
| 196 | 568 |
| 197 | 569 |
| 198 | 570 |
| 199 | 571 |
| 200 | 572 |
| 201 | 573 |
| 202 | 574 |
| 203 | 575 |
| 204 | 576 |
| 205 | 577 |
| 206 | 578 |
| 207 | 579 |
| 208 | 580 |
| 209 | 581 |
| 210 | 582 |
| 211 | 583 |
| 212 | 584 |
| 213 | 585 |
| 214 | 586 |
| 215 | 587 |
| 216 | 588 |
| 217 | 589 |
| 218 | 590 |
| 219 | 591 |
| 220 | 592 |
| 221 | 593 |
| 222 | 594 |
| 223 | 595 |
| 224 | 596 |
| 225 | 597 |
| 226 | 598 |
| 227 | 599 |
| 228 | 600 |
| 229 | 601 |
| 230 | 602 |
| 231 | 603 |
| 232 | 604 |
| 233 | 605 |
| 234 | 606 |
| 235 | 607 |
| 236 | 608 |
| 237 | 609 |
| 238 | 610 |
| 239 | 611 |
| 240 | 612 |
| 241 | 613 |
| 242 | 614 |
| 243 | 615 |
| 244 | 616 |
| 245 | 617 |
| 246 | 618 |
| 247 | 619 |
| 248 | 620 |
| 249 | 621 |
| 250 | 622 |
| 251 | 623 |
| 252 | 624 |
| 253 | 625 |
| 254 | 626 |
| 255 | 627 |
| 256 | 628 |
| 257 | 629 |
| 258 | 630 |
| 259 | 631 |
| 260 | 632 |
| 261 | 633 |
| 262 | 634 |
| 263 | 635 |
| 264 | 636 |
| 265 | 637 |
| 266 | 638 |
| 267 | 639 |
| 268 | 640 |
| 269 | 641 |
| 270 | 642 |
| 271 | 643 |
| 272 | 644 |
| 273 | 645 |
| 274 | 646 |
| 275 | 647 |
| 276 | 648 |
| 277 | 649 |
| 278 | 650 |
| 279 | 651 |
| 280 | 652 |
| 281 | 653 |
| 282 | 654 |
| 283 | 655 |
| 284 | 656 |
| 285 | 657 |
| 286 | 658 |
| 287 | 659 |
| 288 | 660 |
| 289 | 661 |
| 290 | 662 |
| 291 | 663 |
| 292 | 664 |
| 293 | 665 |
| 294 | 666 |
| 295 | 667 |
| 296 | 668 |
| 297 | 669 |
| 298 | 671 |
| 299 | 672 |
| 300 | 673 |
| 301 | 674 |
| 302 | 675 |
| 303 | 676 |
| 304 | 677 |
| 305 | 678 |
| 306 | 679 |
| 307 | 680 |
| 308 | 681 |
| 309 | 682 |
| 310 | 683 |
| 311 | 684 |
| 312 | 685 |
| 313 | 686 |
| 314 | 687 |
| 315 | 688 |
| 316 | 689 |
| 317 | 690 |
| 318 | 691 |
| 319 | 692 |
| 320 | 693 |
| 321 | 694 |
| 2356 | 701 |
| 2357 | 702 |
| 2358 | 703 |
| 2359 | 704 |
| 2360 | 705 |
| 2361 | 706 |
| 2362 | 707 |
| 2363 | 708 |
| 2364 | 709 |
| 2365 | 710 |
| 2366 | 711 |
| 2367 | 712 |
| 2368 | 713 |
| 2369 | 714 |
| 2370 | 715 |
| 2371 | 716 |
| 2372 | 717 |
| 2373 | 718 |
| 2374 | 719 |
| 2375 | 720 |
| 2376 | 721 |
| 2377 | 722 |
| 2378 | 723 |
| 2379 | 724 |
| 2380 | 725 |
| 2381 | 726 |
| 2382 | 727 |
| 2383 | 728 |
| 2384 | 729 |
| 2385 | 730 |
| 2386 | 731 |
| 2387 | 732 |
| 2388 | 733 |
| 2389 | 734 |
| 2390 | 735 |
| 2391 | 736 |
| 2392 | 737 |
| 2393 | 738 |
| 2394 | 739 |
| 2395 | 740 |
| 2396 | 741 |
| 2397 | 742 |
| 2398 | 743 |
| 2399 | 744 |
| 2400 | 745 |
| 2401 | 746 |
| 2402 | 747 |
| 2403 | 748 |
| 2404 | 749 |
| 2405 | 750 |
| 2406 | 751 |
| 2407 | 752 |
| 2408 | 753 |
| 2409 | 754 |
| 2410 | 755 |
| 2411 | 756 |
| 2412 | 757 |
| 2413 | 758 |
| 2414 | 759 |
| 2415 | 760 |
| 2416 | 761 |
| 2417 | 762 |
| 2418 | 763 |
| 2419 | 764 |
| 2420 | 765 |
| 2421 | 766 |
| 2422 | 767 |
| 2423 | 768 |
| 2424 | 769 |
| 2425 | 770 |
| 2426 | 777 |
| 2427 | 778 |
| 2428 | 779 |
| 2429 | 780 |
| 2430 | 781 |
| 2431 | 782 |
| 2432 | 783 |
| 2433 | 784 |
| 2434 | 785 |
| 2435 | 786 |
| 2436 | 787 |
| 2437 | 788 |
| 2438 | 789 |
| 2439 | 790 |
| 2440 | 791 |
| 2441 | 793 |
| 2442 | 794 |
| 2443 | 795 |
| 2444 | 796 |
| 2445 | 797 |
| 2446 | 798 |
| 2447 | 799 |
| 2448 | 800 |
| 2449 | 801 |
| 2450 | 802 |
| 2451 | 803 |
| 2452 | 804 |
| 2453 | 805 |
| 2454 | 806 |
| 2455 | 807 |
| 2456 | 808 |
| 2457 | 809 |
| 2458 | 810 |
| 2459 | 811 |
| 2460 | 812 |
| 2461 | 813 |
| 2462 | 814 |
| 2463 | 815 |
| 2464 | 816 |
| 2465 | 817 |
| 2466 | 818 |
| 2467 | 819 |
| 2468 | 820 |
| 2469 | 821 |
| 2470 | 822 |
| 2471 | 823 |
| 2472 | 824 |
| 2473 | 825 |
| 2474 | 826 |
| 2475 | 827 |
| 2476 | 828 |
| 2477 | 829 |
| 2478 | 830 |
| 2479 | 831 |
| 2480 | 832 |
| 2481 | 833 |
| 2482 | 834 |
| 2483 | 835 |
| 2484 | 836 |
| 2485 | 837 |
| 2486 | 838 |
| 2487 | 839 |
| 2488 | 840 |

In some embodiments, a CasX variant sequence comprises a sequence of SEQ ID NOS: 49-71, presented in the sequence listing which accompanies the instant specification.

e. Class 2 Type V, CasX Variants Derived from Other Class 2 Type V, CasX Variants

In further iterations of the generation of variant proteins, a variant protein can be utilized to generate additional CasX variants of the disclosure. For example, CasX 119 (SEQ ID NO: 72), CasX 491 (SEQ ID NO: 138), and CasX 515 (SEQ ID NO: 145) are exemplary variant proteins that are modified to generate additional CasX variants of the disclosure having improvements or additional properties relative to a reference CasX or CasX variants from which they were derived. CasX 119 contains a substitution of L379R, a substitution of A708K and a deletion of P at position 793 of SEQ ID NO: 2. CasX 491 contains an NTSB and Helical 1B domain swap from SEQ ID NO: 1. CasX 515 was derived from CasX 491 by insertion of P at position 793 (relative to SEQ ID NO: 2) and was used to create additional CasX variants. For example, CasX 668 has an insertion of R at position 26 and a substitution of G223S relative to CasX 515. CasX 672 has substitutions of L169K and G223S relative to CasX 515. CasX 676 has substitutions of L169K and G223S and an insertion of R at position 26 relative to CasX 515.

Exemplary methods used to generate and evaluate CasX variants derived from other CasX variants are described in the Examples, which were created by introducing modifications to the encoding sequence resulting in amino acid substitutions, deletions, or insertions at one or more positions in one or more domains of the CasX variant. The Examples describe the methods used to create variants of CasX 515 (SEQ ID NO: 145) that were then assayed to determine those positions in the sequence that, when modified by an amino acid insertion, deletion or substitution, resulted in an enrichment or improvement in the assays. For purposes of the disclosure, the sequences of the domains of CasX 515 are provided in Table 6 and include an OBD-I domain having the sequence of SEQ ID NO: 2489, an OBD-II domain having the sequence of SEQ ID NO: 2494, NTSB domain having the sequence of SEQ ID NO: 2491, a helical I-I domain having the sequence of SEQ ID NO: 2490, a helical I-II domain having the sequence of SEQ ID NO: 2492, a helical II domain having the sequence of SEQ ID NO: 2493, a RuvC-I domain having the sequence of SEQ ID NO: 2495, a RuvC-II domain having the sequence of SEQ ID NO: 2497, and a TSL domain having the sequence of SEQ ID NO: 2496. By the methods of the disclosure, individual positions in the domains of CasX 515 were modified, assayed, and the resulting positions and exemplary modifications leading to an enrichment or improvement that follow are provided, relative to their position in each domain or subdomain. In some cases, such positions are disclosed in Tables 49-52 of the Examples. In some embodiments, the disclosure provides CasX variants derived from CasX 515 comprising one or more modifications (i.e., an insertion, a deletion, or a substitution) at one or more amino acid positions in the NTSB domain relative to SEQ ID NO: 2491 selected from the group consisting of P2, S4, Q9, E15, G20, G33, L41, Y51, F55, L68, A70, E75, K88, and G90, wherein the modification results in an improved characteristic relative to CasX 515. In a particular embodiment, the one or more modifications at one or more amino acid positions in the NTSB domain relative to SEQ ID NO: 14533 are selected from the group consisting of \hat{ }G2, \hat{ }I4, \hat{ }L4, Q9P, E15S, G20D, [S30], G33T, L41A, Y51T, F55V, L68D, L68E, L68K, A70Y, A70S, E75A, E75D, E75P, K88Q, and G90Q (where “\hat{ }” represents and insertion and “[ ]” represents a deletion at that position). In some embodiments, the disclosure provides CasX variants derived from CasX 515 comprising one or more modifications at one or more amino acid positions in the helical I-II domain relative to SEQ ID NO: 2492 selected from the group consisting of 124, A25, Y29 G32, G44, S48, S51, Q54, 156, V63, S73, L74, K97, V100, M112, L116, G137, F138, and S140, wherein the modification results in an improved characteristic relative to CasX 515. In a particular embodiment, the one or more modifications at one or more amino acid positions in the helical I-II domain are selected from the group consisting of \hat{ }T24, \hat{ }C25, Y29F, G32Y, G32N, G32H, G32S, G32T, G32A, G32V, [G32], G32S, G32T, G44L, G44H, S48H, S48T, S51T, Q54H, I56T, V63T, S73H, L74Y, K97G, K97S, K97D, K97E, V100L, M112T, M112W, M112R, M112K, L116K, G137R, G137K, G137N, \hat{ }Q138, and S140Q. In some embodiments, the disclosure provides CasX variants derived from CasX 515 comprising one or more modifications at one or more amino acid positions in the helical II domain relative to SEQ ID NO: 2493 selected from the group consisting of L2, V3, E4, R5, Q6, A7, E9, V10, D1i, W12, W13, D14, M15, V16, C17, N18, V19, K₂O, L22, 123, E25, K26, K31, Q35, L37, A38, K41, R42, Q43, E44, L46, K57, Y65, G68, L70, L71, L72, E75, G79, D81, W82, K84, V85, Y86, D87, 193, K95, K96, E98, L100, K102, 1104, K105, E109, R110, D114, K118, A120, L121, W124, L125, R126, A127, A129, 1133, E134, G135, L136, E138, D140, K141, D142, E143, F144, C145, C147, E148, L149, K150, L151, Q152, K153, L158, E166, and A167, wherein the modification results in an improved characteristic relative to CasX 515. In a particular embodiment, the one or more modifications at one or more amino acid positions in the helical II domain are selected from the group consisting of \hat{ }A2, \hat{ }H2, [L2]+[V3], V3E, V3Q, V3F, [V3], \hat{ }D3, V3P, E4P, [E4], E4D, E4L, E4R, R5N, Q6V, \hat{ }Q6, \hat{ }G7, \hat{ }H9, \hat{ }A9, VD10, \hat{ }T10, [V10], \hat{ }F10, \hat{ }D11, [D11], D11S, [W12], W12T, W12H, \hat{ }P12, \hat{ }Q13, \hat{ }G12, \hat{ }R13, W13P, W13D, \hat{ }D13, W13L, \hat{ }P14, \hat{ }D14, [D14]+[M15], [M15], \hat{ }T16, \hat{ }P17, N18I, V19N, V19H, K20D, L22D, I23S, E25C, E25P, \hat{ }G25, K26T, K27E, K31L, K31Y, Q35D, Q35P, \hat{ }S37, [L37]+[A38], K41L, \hat{ }R42, [Q43]+[E44], L46N, K57Q, Y65T, G68M, L70V, L71C, L72D, L72N, L72W, L72Y, E75F, E75L, E75Y, G79P, \hat{ }E79, \hat{ }T81, \hat{ }R81, \hat{ }W81, \hat{ }Y81, \hat{ }W82, \hat{ }Y82, W82G, W82R, K84D, K84H, K84P, K84T, V85L, V85A, \hat{ }I85, Y86C, D87G, D87M, D87P, I93C, K95T, K96R, E98G, L100A, K102H, I104T, I104S, I104Q, K105D, \hat{ }K109, E109L, R110D, [R110], D114E, \hat{ }D114, K118P, A120R, L121T, W124L, L125C, R126D, A127E, A127L, A129T, A129K, I133E, \hat{ }C133, \hat{ }S134, \hat{ }G134, \hat{ }R135, G135P, L136K, L136D, L136S, L136H, [E138], D140R, \hat{ }D140, \hat{ }P141, \hat{ }D142, [E143]+[F144], \hat{ }Q143, F144K, [F144], [F144]+[C145], C145R, \hat{ }G145, C145K, C147D, \hat{ }V148, E148D, \hat{ }H149, L149R, K150R, L151H, Q152C, K153P, L158S, E166L, and \hat{ }F167. In some embodiments, the disclosure provides CasX variants derived from CasX 515 comprising one or more modifications at one or more amino acid positions in the RuvC-I domain relative to SEQ ID NO: 2495 selected from the group consisting of 14, K5, P6, M7, N8, L9, V12, G49, K63, K80, N83, R90, M125, and L146, wherein the modification results in an improved characteristic relative to CasX 515. In a particular embodiment, the one or more modifications at one or more amino acid positions in the RuvC-I domain are selected from the group consisting of \hat{ }I4, \hat{ }S5, \hat{ }T6, \hat{ }N6, \hat{ }R7, \hat{ }K7, \hat{ }H8, \hat{ }S8, V12L, G49W, G49R, S51R, S51K, K62S, K62T, K62E, V65A, K80E, N83G, R90H, R90G, M125S, M125A, L137Y, \hat{ }P137, [L141], L141R, L141D, \hat{ }Q142, \hat{ }R143, \hat{ }N143, E144N, \hat{ }P146, L146F, P147A, K149Q, T150V, \hat{ }R152, \hat{ }H153, T155Q, \hat{ }H155, \hat{ }R155, \hat{ }L156, [L156], \hat{ }W156, \hat{ }A157, \hat{ }F157, A157S, Q158K, [Y159], T160Y, T160F, \hat{ }I161, S161P, T163P, \hat{ }N163, C164K, and C164M. In some embodiments, the disclosure provides CasX variants derived from CasX 515 comprising one or more modifications at one or more amino acid positions in the OBD-I domain relative to SEQ ID NO: 2489 selected from the group consisting of 14, K5, P6, M7, N8, L9, V12, G49, K63, K80, N83, R90, M125, and L146, wherein the modification results in an improved characteristic relative to CasX 515. In a particular embodiment, the one or more modifications at one or more amino acid positions in the OBD-I domain are selected from the group consisting of \hat{ }G3, I3G, I3E, \hat{ }G4, K4G, K4P, K4S, K4W, K4W, R5P, \hat{ }P5, \hat{ }G5, R5S, \hat{ }S5, R5A, R5P, R5G, R5L, I6A, I6L, \hat{ }G6, N7Q, N7L, N7S, K8G, K15F, D16W, \hat{ }F16, \hat{ }F18, \hat{ }P17, M28P, M28H, V33T, R34P, M36Y, R41P, L47P, \hat{ }P48, E52P, \hat{ }P55, [P55]+[Q56], Q56S, Q56P, \hat{ }D56, \hat{ }T56, and Q56P. In some embodiments, the disclosure provides CasX variants derived from CasX 515 comprising one or more modifications at one or more amino acid positions in the OBD-II domain relative to SEQ ID NO: 2494 selected from the group consisting of 14, K5, P6, M7, N8, L9, V12, G49, K63, K80, N83, R90, M125, and L146, wherein the modification results in an improved characteristic relative to CasX 515. In a particular embodiment, the one or more modifications at one or more amino acid positions in the OBD-I domain are selected from the group consisting of [S2], I3R, I3K, [I3]+[L4], [L4], K11T, \hat{ }P24, K37G, R42E, \hat{ }S53, \hat{ }R58, [K63], M70T, I82T, Q92I, Q92F, Q92V, Q92A, \hat{ }A93, K110Q, R115Q, L121T, \hat{ }A124, \hat{ }R141, \hat{ }D143, \hat{ }A143, \hat{ }W144, and \hat{ }A145. In some embodiments, the disclosure provides CasX variants derived from CasX 515 comprising one or more modifications at one or more amino acid positions in the TSL domain relative to SEQ ID NO: 2496 selected from the group consisting of S1, N2, C3, G4, F5, 17, K18, V58, S67, T76, G78, S80, G81, E82, S85, V96, and E98, wherein the modification results in an improved characteristic relative to CasX 515. In a particular embodiment, the one or more modifications at one or more amino acid positions in the OBD-I domain are selected from the group consisting of \hat{ }M1, [N2], \hat{ }V2, C3S, \hat{ }G4, \hat{ }W4, F5P, \hat{ }W7, K18G, V58D, \hat{ }A67, T76E, T76D, T76N, G78D, [S80], [G81], \hat{ }E82, \hat{ }N82, S85I, V96C, V96T, and E98D. It will be understood that combinations of any of the same foregoing modifications of the paragraph can similarly be introduced into the CasX variants of the disclosure, resulting in a CasX variant with improved characteristics. For example, in one embodiment, the disclosure provides CasX variant 535 (SEQ ID NO: 164), which has a single mutation of G223S relative to CasX 515. In another embodiment, the disclosure provides CasX variant 668 (SEQ ID NO: 296), which has an insertion of R at position 26 and a substitution of G223S relative to CasX 515. In another embodiment, the disclosure provides CasX 672 (SEQ ID NO: 299), which has substitutions of L169K and G223S relative to CasX 515. In another embodiment, the disclosure provides CasX 676 (SEQ ID NO: 303), which has substitutions of L169K and G223S and an insertion of R at position 26 relative to CasX 515. CasX variants with improved characteristics relative to CasX 515 include variants of Table 5.

Exemplary characteristics that can be improved in CasX variant proteins relative to the same characteristics in reference CasX proteins or relative to the CasX variant from which they were derived include, but are not limited to improved folding of the variant, increased binding affinity to the gRNA, increased binding affinity to the target nucleic acid, improved ability to utilize a greater spectrum of PAM sequences in the editing and/or binding of target nucleic acid, improved unwinding of the target DNA, increased editing activity, improved editing efficiency, improved editing specificity for the target nucleic acid, decreased off-target editing or cleavage, increased percentage of a eukaryotic genome that can be efficiently edited, increased activity of the nuclease, increased target strand loading for double strand cleavage, decreased target strand loading for single strand nicking, increased binding of the non-target strand of DNA, improved protein stability, improved protein:gRNA (RNP) complex stability, and improved fusion characteristics. In a particular embodiment, as described in the Examples, such improved characteristics can include, but are not limited to, improved cleavage activity in target nucleic acids having TTC, ATC, and CTC PAM sequences, increased specificity for cleavage of a target nucleic acid sequence, and decreased off-target cleavage of a target nucleic acid.

TABLE 6
CasX 515 domain sequences

| | SEQ | |
|---|---|---|
| | ID | |
| Domain | NO | Amino Acid Sequence |
| ODB-I | 2489 | QEIKRINKIRRRLVKDSNTKKAGKTGPMKTLLVR |
| | | VMTPDLRERLENLRKKPENIPQ |
| Helical | 2490 | PISNTSRANLNKLLTDYTEMKKAILHVYWEEFQK |
| I-I | | DPVGLMSRVA |
| NTSB | 2491 | QPASKKIDQNKLKPEMDEKGNLTTAGFACSQCGQ |
| | | PLFVYKLEQVSEKGKAYTNYFGRCNVAEHEKLIL |
| | | LAQLKPEKDSDEAVTYSLGKFGQ |
| Helical | 2492 | RALDFYSIHVTKESTHPVKPLAQIAGNRYASGPV |
| I-II | | GKALSDACMGTIASFLSKYQDIIIEHQKVVKGNQ |
| | | KRLESLRELAGKENLEYPSVTLPPQPHTKEGVDA |
| | | YNEVIARVRMWVNLNLWQKLKLSRDDAKPLLRLK |
| | | GFPSF |
| Helical | 2493 | PLVERQANEVDWWDMVCNVKKLINEKKEDGKVFW |
| II | | QNLAGYKRQEALRPYLSSEEDRKKGKKFARYQLG |
| | | DLLLHLEKKHGEDWGKVYDEAWERIDKKVEGLSK |
| | | HIKLEEERRSEDAQSKAALTDWLRAKASFVIEGL |
| | | KEADKDEFCRCELKLQKWYGDLRGKPFAIEAE |
| OBD-II | 2494 | NSILDISGFSKQYNCAFIWQKDGVKKLNLYLIIN |
| | | YFKGGKLRFKKIKPEAFEANRFYTVINKKSGEIV |
| | | PMEVNFNFDDPNLIILPLAFGKRQGREFIWNDLL |
| | | SLETGSLKLANGRVIEKTLYNRRTRQDEPALFVA |
| | | LTFERREVLD |
| RuvC-I | 2495 | SSNIKPMNLIGVDRGENIPAVIALTDPEGCPLSR |
| | | FKDSLGNPTHILRIGESYKEKQRTIQAKKEVEQR |
| | | RAGGYSRKYASKAKNLADDMVRNTARDLLYYAVT |
| | | QDAMLIFENLSRGFGRQGKRTFMAERQYTRMEDW |
| | | LTAKLAYEGLPSKTYLSKTLAQYTSKTC |
| TSL | 2496 | SNCGFTITSADYDRVLEKLKKTATGWMTTINGKE |
| | | LKVEGQITYYNRYKRQNVVKDLSVELDRLSEESV |
| | | NNDISSWTKGRSGEALSLLKKRFSHRPVQEKFVC |
| | | LNCGFETH |
| RuvC-II | 2497 | ADEQAALNIARSWLFLRSQEYKKYQTNKTTGNTD |
| | | KRAFVETWQSFYRKKLKEVWKPAV |

f. CasX Fusion Proteins

In some embodiments, the disclosure provides SIRV and siAAV systems encoding CasX proteins comprising a heterologous protein fused to the CasX. In some cases, the CasX is a reference CasX protein. In other cases, the CasX is a CasX variant of any of the embodiments described herein. This includes CasX comprising N-terminal, C-terminal, or internal fusions of the CasX to a heterologous protein or domain thereof. In some embodiments, the CasX protein is fused to one or more proteins or domains thereof that has a different activity of interest, resulting in a fusion protein. For example, in some embodiments, the CasX protein is fused to a protein (or domain thereof) that inhibits transcription, modifies a target nucleic acid, or modifies a polypeptide associated with a nucleic acid (e.g., histone modification). Examples of such fusion partners contemplated for use in the CasX of the disclosure are described in WO2022120095, incorporated by reference herein.

A variety of heterologous polypeptides are suitable for inclusion in a reference CasX or CasX variant fusion protein for use in the SIRV and siAAV systems of the disclosure. In some cases, the fusion partner can modulate transcription (e.g., inhibit transcription, increase transcription) of a target DNA. For example, in some cases the fusion partner is a protein (or a domain from a protein) that inhibits transcription (e.g., a transcriptional repressor, a protein that functions via recruitment of transcription inhibitor proteins, modification of target DNA such as methylation, recruitment of a DNA modifier, modulation of histones associated with target DNA, recruitment of a histone modifier such as those that modify acetylation and/or methylation of histones, and the like). In some cases, the fusion partner is a protein (or a domain from a protein) that increases transcription (e.g., a transcription activator, a protein that acts via recruitment of transcription activator proteins, modification of target DNA such as demethylation, recruitment of a DNA modifier, modulation of histones associated with target DNA, recruitment of a histone modifier such as those that modify acetylation and/or methylation of histones, and the like).

In some cases, a fusion partner has enzymatic activity that modifies a target nucleic acid sequence; e.g., nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity.

In some cases, a fusion partner has enzymatic activity that modifies a polypeptide (e.g., a histone) associated with a target nucleic acid (e.g., methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity).

Examples of proteins (or fragments thereof) that can be used as a fusion partner to increase transcription include but are not limited to: transcriptional activators such as VP16, VP64, VP48, VP160, p65 subdomain (e.g., from NFkB), and activation domain of EDLL and/or TAL activation domain (e.g., for activity in plants); histone lysine methyltransferases such as SET1A, SET1B, MLL1 to 5, ASH1, SYMD2, NSD1, and the like; histone lysine demethylases such as JHDM2a/b, UTX, JMJD3, and the like; histone acetyltransferases such as GCN5, PCAF, CBP, p300, TAF1, TIP60/PLIP, MOZ/MYST3, MORF/MYST4, SRC1, ACTR, P160, CLOCK, and the like; and DNA demethylases such as Ten-Eleven Translocation (TET) dioxygenase 1 (TET1CD), TET1, DME, DML1, DML2, ROS1, and the like.

Examples of proteins (or fragments thereof) that can be used as a fusion partner to decrease transcription include but are not limited to: transcriptional repressors such as the Kruppel associated box (KRAB or SKD); KOX1 repression domain; the Mad mSIN3 interaction domain (SID); the ERF repressor domain (ERD), the SRDX repression domain (e.g., for repression in plants), and the like; histone lysine methyltransferases such as Pr-SET7/8, SUV4-20H1, RIZ1, and the like; histone lysine demethylases such as JMJD2A/JHDM3A, JMJD2B, JMJD2C/GASC1, JMJD2D, JARID1A/RBP2, JARID1B/PLU-1, JARID 1C/SMCX, JARID1D/SMCY, and the like; histone lysine deacetylases such as HDAC1, HDAC2, HDAC3, HDAC8, HDAC4, HDAC5, HDAC7, HDAC9, SIRT1, SIRT2, HDAC11, and the like; DNA methylases such as HhaI DNA m5c-methyltransferase (M.HhaI), DNA methyltransferase 1 (DNMT1), DNA methyltransferase 3a (DNMT3a), DNA methyltransferase 3b (DNMT3b), METI, DRM3 (plants), ZMET2, CMT1, CMT2 (plants), and the like; and periphery recruitment elements such as Lamin A, Lamin B, and the like.

In some cases, a CasX variant protein of the present disclosure for use in the SIRV systems can include an endosomal escape peptide. In some cases, an endosomal escape polypeptide comprises the amino acid sequence GLFXALLXLLXSLWXLLLXA (SEQ ID NO: 48), wherein each X is independently selected from lysine, histidine, and arginine. In some cases, an endosomal escape polypeptide comprises the amino acid sequence GLFHALLHLLHSLWHLLLHA (SEQ ID NO: 342), or HHHHHHHHH (SEQ ID NO: 343).

In some cases, a heterologous polypeptide (a fusion partner) for use in the SIRV and siAAV systems provides for subcellular localization; i.e., the heterologous polypeptide contains a subcellular localization sequence (e.g., a nuclear localization signal (NLS) for targeting to the nucleus, a sequence to keep the fusion protein out of the nucleus, e.g., a nuclear export sequence (NES), a sequence to keep the fusion protein retained in the cytoplasm, a mitochondrial localization signal for targeting to the mitochondria, a chloroplast localization signal for targeting to a chloroplast, an ER retention signal, and the like). In some embodiments, a subject RNA-guided polypeptide or a conditionally active RNA-guided polypeptide and/or subject CasX fusion protein does not include a NLS so that the protein is not targeted to the nucleus (which can be advantageous, e.g., when the target nucleic acid sequence is an RNA that is present in the cytosol). In some embodiments, a fusion partner can provide a tag (i.e., the heterologous polypeptide is a detectable label) for ease of tracking and/or purification (e.g., a fluorescent protein, e.g., green fluorescent protein (GFP), yellow fluorescent protein (YFP), red fluorescent protein (RFP), cyan fluorescent protein (CFP), mCherry, tdTomato, and the like; a histidine tag, e.g., a 6×His tag; a hemagglutinin (HA) tag; a FLAG tag; a Myc tag; and the like).

In some embodiments, sequences encoding one or more NLS are incorporated into the SIRV and siAAV constructs. In some embodiments, the one or more NLS are incorporated at or near the C-terminus of the CasX protein. In some embodiments, the one or more NLS are expressed at or near the N-terminus of the CasX protein. In other embodiments, the one or more NLS located at or near the N-terminus and at or near the C-terminus of the CasX protein.

In some cases, non-limiting examples of NLSs suitable for use with a CasX variant include sequences having at least about 80%, at least about 90%, or at least about 95% identity or are identical to sequences derived from: the NLS of the SV40 virus large T-antigen, having the amino acid sequence PKKKRKV (SEQ ID NO: 344); the NLS from nucleoplasmin (e.g., the nucleoplasmin bipartite NLS with the sequence KRPAATKKAGQAKKKK (SEQ ID NO: 345); the c-myc NLS having the amino acid sequence PAAKRVKLD (SEQ ID NO: 346) or RQRRNELKRSP (SEQ ID NO: 347); the hRNPAl M9 NLS having the sequence NQSSNFGPMKGGNFGGRSSGPYGGGGQYFAKPRNQGGY (SEQ ID NO: 348); the sequence RMRIZFKNKGKDTAELRRRRVEVSVELRKAKKDEQILKRRNV (SEQ ID NO: 349) of the IBB domain from importin-alpha; the sequences VSRKRPRP (SEQ ID NO: 350) and PPKKARED (SEQ ID NO: 351) of the myoma T protein; the sequence PQPKKKPL (SEQ ID NO: 352) of human p53; the sequence SALIKKKKKMAP (SEQ ID NO: 353) of mouse c-abl IV; the sequences DRLRR (SEQ ID NO: 354) and PKQKKRK (SEQ ID NO: 355) of the influenza virus NS1; the sequence RKLKKKIKKL (SEQ ID NO: 356) of the Hepatitis virus delta antigen; the sequence REKKKFLKRR (SEQ ID NO: 357) of the mouse Mxl protein; the sequence KRKGDEVDGVDEVAKKKSKK (SEQ ID NO: 358) of the human poly(ADP-ribose) polymerase; the sequence RKCLQAGMNLEARKTKK (SEQ ID NO: 359) of the steroid hormone receptors (human) glucocorticoid; the sequence PRPRKIPR (SEQ ID NO: 360) of Borna disease virus P protein (BDV-P1); the sequence PPRKKRTVV (SEQ ID NO: 361) of hepatitis C virus nonstructural protein (HCV-NS5A); the sequence NLSKKKKRKREK (SEQ ID NO: 362) of LEF1; the sequence RRPSRPFRKP (SEQ ID NO: 363) of ORF57 simirae; the sequence KRPRSPSS (SEQ ID NO: 364) of EBV LANA; the sequence KRGINDRNFWRGENERKTR (SEQ ID NO: 365) of Influenza A protein; the sequence PRPPKMARYDN (SEQ ID NO: 366) of human RNA helicase A (RHA); the sequence KRSFSKAF (SEQ ID NO: 367) of nucleolar RNA helicase II; the sequence KLKIKRPVK (SEQ ID NO: 368) of TUS-protein; the sequence PKKKRKVPPPPAAKRVKLD (SEQ ID NO: 369) associated with importin-alpha; the sequence PKTRRRPRRSQRKRPPT (SEQ ID NO: 370) from the Rex protein in HTLV-1; the sequence SRRRKANPTKLSENAKKLAKEVEN (SEQ ID NO: 371) from the EGL-13 protein of Caenorhabditis elegans; and the sequences KTRRRPRRSQRKRPPT (SEQ ID NO: 372), RRKKRRPRRKKRR (SEQ ID NO: 373), PKKKSRKPKKKSRK (SEQ ID NO: 374), HKKKHPDASVNFSEFSK (SEQ ID NO: 375), QRPGPYDRPQRPGPYDRP (SEQ ID NO: 376), LSPSLSPLLSPSLSPL (SEQ ID NO: 377), RGKGGKGLGKGGAKRHRK (SEQ ID NO: 378), PKRGRGRPKRGRGR (SEQ ID NO: 379), PKKKRKVPPPPAAKRVKLD (SEQ ID NO: 380), PKKKRKVPPPPKKKRKV (SEQ ID NO: 381), PAKRARRGYKC (SEQ ID NO: 382), KLGPRKATGRW (SEQ ID NO: 383), PRRKREE (SEQ ID NO: 384), PYRGRKE (SEQ ID NO: 385), PLRKRPRR (SEQ ID NO: 386), PLRKRPRRGSPLRKRPRR (SEQ ID NO: 387), PAAKRVKLDGGKRTADGSEFESPKKKRKV (SEQ ID NO: 388), PAAKRVKLDGGKRTADGSEFESPKKKRKVGIHGVPAA (SEQ ID NO: 389), PAAKRVKLDGGKRTADGSEFESPKKKRKVAEAAAKEAAAKEAAAKA (SEQ ID NO: 390), PAAKRVKLDGGKRTADGSEFESPKKKRKVPG (SEQ ID NO: 391), KRKGSPERGERKRHW (SEQ ID NO: 392), KRTADSQHSTPPKTKRKVEFEPKKKRKV (SEQ ID NO: 393), PKKKRKVGGSKRTADSQHSTPPKTKRKVEFEPKKKRKV (SEQ ID NO: 394), MAPKKKRKVSR (SEQ ID NO: 771), and MAPKKKRKVGGSPKKKRKVGGSPKKKRKVGGSPKKKRKVSR (SEQ ID NO: 772). In some cases, NLSs suitable for use with a CasX variant include sequences having at least about 80%, at least about 90%, or at least about 95% identity or are identical to SEQ ID NOS: 538-613. In some embodiments, the one or more NLS are linked to the CRISPR protein or to adjacent NLS with a linker peptide wherein the linker peptide is selected from the group consisting of RS, (G)n (SEQ ID NO: 395), (GS)n (SEQ ID NO: 396), (GSGGS)n (SEQ ID NO: 397), (GGSGGS)n (SEQ ID NO: 398), (GGGS)n (SEQ ID NO: 399), GGSG (SEQ ID NO: 400), GGSGG (SEQ ID NO: 401), GSGSG (SEQ ID NO: 402), GSGGG (SEQ ID NO: 403), GGGSG (SEQ ID NO: 404), GSSSG (SEQ ID NO: 405), GPGP (SEQ ID NO: 406), GGP, PPP, PPAPPA (SEQ ID NO: 407), PPPG (SEQ ID NO: 408), PPPGPPP (SEQ ID NO: 409), PPP(GGGS)n (SEQ ID NO: 410), (GGGS)nPPP (SEQ ID NO: 411), AEAAAKEAAAKEAAAKA (SEQ ID NO: 412), and TPPKTKRKVEFE (SEQ ID NO: 413), where n is 1 to 5. In general, NLS (or multiple NLSs) are of sufficient strength to drive accumulation of a reference or CasX variant fusion protein in the nucleus of a eukaryotic cell. Detection of accumulation in the nucleus may be performed by any suitable technique. For example, a detectable marker may be fused to a reference or CasX variant fusion protein such that location within a cell may be visualized. Cell nuclei may also be isolated from cells, the contents of which may then be analyzed by any suitable process for detecting protein, such as immunohistochemistry, western blot, or enzyme activity assay. Accumulation in the nucleus may also be determined indirectly. In some embodiments, an NLS suitable for use with a CasX variant include any of the sequences of Tables 7, 22, or 23.

The disclosure contemplates assembly of multiple NLS in various configurations for linkage to the CRISPR protein utilized in the SIRV and siAAV of the embodiments described herein. In some embodiments, 1, 2, 3, 4 or more NLS are linked by linker peptides at or near (e.g., within 50 amino acids of) the N-terminus of the CRISPR protein. In other embodiments, 1, 2, 3, 4 or more NLS are linked by linker peptides at or near (e.g., within 50 amino acids of) the C-terminus of the CRISPR protein. In some embodiments, the NLS linked to the N-terminus of the CRISPR protein are identical to the NLS linked to the C-terminus. In other embodiments, the NLS linked to the N-terminus of the CRISPR protein are different to the NLS linked to the C-terminus. In some embodiments, the NLS linked to the N-terminus of the CRISPR protein are selected from the group consisting of the N-terminal sequences as set forth in Table 7 and Table 22. In some embodiments, the NLS linked to the C-terminus of the CRISPR protein are selected from the group consisting of the C-terminal sequences as set forth in Table 7 and Table 23. Detection of accumulation in the nucleus of the CasX variant protein enhanced by the addition of NLS may be performed by any suitable technique; e.g., a detectable marker may be fused to a reference or CasX variant fusion protein such that location within a cell may be visualized by any suitable process for detecting protein, such as immunohistochemistry, Western blot, or enzyme activity assay. Accumulation in the nucleus may also be determined indirectly.

TABLE 7
NLS Sequences

| N Terminal Sequence | C Terminal Sequence |
|---|---|
| (SEQ ID NO) | (SEQ ID NO) |
| 2498 | 2545 |
| 2499 | 2546 |
| 2500 | 2547 |
| 2501 | 2548 |
| 2502 | 2549 |
| 2503 | 2550 |
| 2504 | 2551 |
| 2505 | 2552 |
| 2506 | 2553 |
| 2507 | 2554 |
| 2508 | 2555 |
| 2509 | 2556 |
| 2510 | 2557 |
| 2511 | 2558 |
| 2512 | 2559 |
| 2513 | 2560 |
| 2514 | 2561 |
| 2515 | 2562 |
| 2516 | 2563 |
| 2517 | 2564 |
| 2518 | 2565 |
| 2519 | 2566 |
| 2520 | 2567 |
| 2521 | 2568 |
| 2522 | 2569 |
| 2523 | 2570 |
| 2524 | 2571 |
| 2525 | 2572 |
| 2526 | 2573 |
| 2527 | 2574 |
| 2528 | 2575 |
| 2529 | 2576 |
| 2530 | 2577 |
| 2531 | 2578 |
| 2532 | 2579 |
| 2533 | 2580 |
| 2534 | 2581 |
| 2535 | 2582 |
| 2536 | 2583 |
| 2537 | 2584 |
| 2538 | 2585 |
| 2539 | 2586 |
| 2540 | 2587 |
| 2541 | 2588 |
| 2542 | 2589 |
| 2543 | 2590 |
| 2544 | 2591 |

In some cases, a CasX variant fusion protein for use in the SIRV and siAAV systems includes a “protein transduction domain” or PTD (also known as a CPP—cell penetrating peptide), which refers to a protein, polynucleotide, carbohydrate, or organic or inorganic compound that facilitates traversing a lipid bilayer, micelle, cell membrane, organelle membrane, or vesicle membrane. A PTD attached to another molecule, which can range from a small polar molecule to a large macromolecule and/or a nanoparticle, facilitates the molecule traversing a membrane, for example going from an extracellular space to an intracellular space, or from the cytosol to within an organelle. In some embodiments, a PTD is covalently linked to the amino terminus of a CasX variant fusion protein. In some embodiments, a PTD is covalently linked to the carboxyl terminus of a CasX variant fusion protein. In some cases, the PTD is inserted internally in the sequence of a CasX variant fusion protein at a suitable insertion site. In some cases, a CasX variant fusion protein includes (is conjugated to, is fused to) one or more PTDs (e.g., two or more, three or more, four or more PTDs). In some cases, a PTD includes one or more nuclear localization signals (NLS). Examples of PTDs include, but are not limited to, peptide transduction domain of HIV TAT comprising YGRKKRRQRRR (SEQ ID NO: 414), RKKRRQRR (SEQ ID NO: 415); YARAAARQARA (SEQ ID NO: 416); THRLPRRRRRR (SEQ ID NO: 417); and GGRRARRRRRR (SEQ ID NO: 418); a polyarginine sequence comprising a number of arginine's sufficient to direct entry into a cell (e.g., 3, 4, 5, 6, 7, 8, 9, 10, or 10-50 arginine's, SEQ ID NO: 419); a VP22 domain (Zender et al. (2002) Cancer Gene Ther. 9(6):489-96); a Drosophila Antennapedia protein transduction domain (Noguchi et al. (2003) Diabetes 52(7): 1732-1737); a truncated human calcitonin peptide (Trehin et al. (2004) Pharm. Research 21:1248-1256); polylysine (Wender et al. (2000) Proc. Natl. Acad. Sci. USA 97: 13003-13008); RRQRRTSKLMKR; Transportan GWTLNSAGYLLGKINLKALAALAKKIL (SEQ ID NO: 420); KALAWEAKLAKALAKALAKHLAKALAKALKCEA (SEQ ID NO: 421); and RQIKIWFQNRRMKWKK (SEQ ID NO: 422). In some embodiments, the PTD is an activatable CPP (ACPP) (Aguilera et al. (2009) Integr Biol (Camb) June; 1(5-6): 371-381). ACPPs comprise a polycationic CPP (e.g., Arg9 or “R9”) connected via a cleavable linker to a matching polyanion (e.g., Glu9 or “E9”), which reduces the net charge to nearly zero and thereby inhibits adhesion and uptake into cells. Upon cleavage of the linker, the polyanion is released, locally unmasking the polyarginine and its inherent adhesiveness, thus “activating” the ACPP to traverse the membrane.

In some embodiments, a CasX variant fusion protein can be linked at the C-terminal and/or N-terminal end to a heterologous polypeptide (fusion partner) via a linker polypeptide (e.g., one or more linker polypeptides). The linker polypeptide may have any of a variety of amino acid sequences. Proteins can be joined by a spacer peptide, generally of a flexible nature, although other chemical linkages are not excluded. Suitable linkers include polypeptides of between 4 amino acids and 40 amino acids in length, or between 4 amino acids and 25 amino acids in length. These linkers are generally produced by using synthetic, linker-encoding oligonucleotides to couple the proteins. Peptide linkers with a degree of flexibility can be used. The linking peptides may have virtually any amino acid sequence, bearing in mind that the preferred linkers will have a sequence that results in a generally flexible peptide. The use of small amino acids, such as glycine and alanine, are of use in creating a flexible peptide. The creation of such sequences is routine to those of skill in the art. A variety of different linkers are commercially available and are considered suitable for use. Exemplary linker polypeptides include peptides selected from the group consisting of RS, (G)n (SEQ ID NO: 395), (GS)n (SEQ ID NO: 396), (GSGGS)n (SEQ ID NO: 397), (GGSGGS)n (SEQ ID NO: 398), (GGGS)n (SEQ ID NO: 399), GGSG (SEQ ID NO: 400), GGSGG (SEQ ID NO: 401), GSGSG (SEQ ID NO: 402), GSGGG (SEQ ID NO: 403), GGGSG (SEQ ID NO: 404), GSSSG (SEQ ID NO: 405), GPGP (SEQ ID NO: 406), GGP, PPP, PPAPPA (SEQ ID NO: 407), PPPG (SEQ ID NO: 408), PPPGPPP (SEQ ID NO: 409), PPP(GGGS)n (SEQ ID NO: 410), (GGGS)nPPP (SEQ ID NO: 411), AEAAAKEAAAKEAAAKA (SEQ ID NO: 412), and TPPKTKRKVEFE (SEQ ID NO: 413), where n is 1 to 5. The ordinarily skilled artisan will recognize that design of a peptide conjugated to any elements described above can include linkers that are all or partially flexible, such that the linker can include a flexible linker as well as one or more portions that confer less flexible structure.

### VI. Self-Inactivating Viral-Derived Particle Systems Comprising SIRV

In another aspect, the present disclosure relates to use of self-inactivating viral-derived particle systems for delivery of SIRV to target cells for modification of target nucleic acid. A number of viral systems can be utilized to package, or contain the SIRV polynucleotide of the embodiments described herein. Such constructs comprise a viral capsid and an SIRV of any one of the embodiments described herein. Upon transduction and delivery of the SIRV polynucleotide to the cell, the CRISPR components, e.g., a Class 2 Type V protein and a guide RNA, are expressed and effect the desired modification of the target nucleic acid, and then via the self-inactivating mechanisms described herein, the polynucleotide is ultimately cleaved, reducing or eliminating the further expression of one or more of the CRISPR components. Non-limiting examples of viral-derived particle systems contemplated for use in the packaging of the SIRV include adeno associated virus (AAV), adenovirus, lentivirus, and gammaretrovirus.

In some embodiments, the disclosure provides siAAV comprising an AAV capsid protein and a polynucleotide comprising components selected from: i) a 5′ adeno-associated virus (AAV) inverted terminal repeat (ITR) sequence; ii) a 3′ AAV ITR sequence; iii) a sequence encoding a Class 2 Type V CRISPR protein; iv) a first promoter operably linked to the sequence encoding the Class 2 Type V CRISPR protein; v) a sequence encoding a first guide RNA (gRNA) comprising a targeting sequence that is complementary to a target nucleic acid of a cell to be modified and complementary to the one or more self-inactivating segments; vi) a second promoter sequence operably linked to the sequence encoding the first gRNA; and vii) one or more self-inactivating segments of the polynucleotide comprising a protospacer adjacent motif (PAM) sequence and a sequence capable of being bound and cleaved by a ribonuclear protein complex (RNP) of the Class 2 Type V CRISPR protein and the first gRNA, wherein the PAM sequence of the one or more self-inactivating segments is different from the PAM sequence of the target nucleic acid of the cell to be modified and promotes less efficient cleavage or rate of cleavage of the self-inactivating segment by the RNP compared to the PAM sequence 5′ and adjacent to the target nucleic acid of the cell to be modified. As previously described, the selection of the PAM sequence of the self-inactivating segments is based on the PAM sequence of the target nucleic acid sequence to be modified, the preference of the CRISPR nuclease utilized, and the rank-order of the strength of the PAM relative to the foregoing; e.g., if the PAM sequence of the target nucleic acid of the cell to be modified is TTC and the PAM preference of the Class 2 Type V CRISPR protein is TTC, then the PAM sequence of the one or more self-inactivating segments is selected from the group consisting of ATC, CTC, TTT, GTT, and GTC. In some embodiments, the polynucleotide comprising the components of (i)-(vii), above, comprises a sequence selected from the group consisting of SEQ ID NOs 4151-4156, or a sequence having at least about 70%, at least about 80%, at least about 80%, at least about 85%, at least about 90%, or at least about 95%, or at least about 95%, or at least about 96%, or at least about 97%, or at least about 98%, or at least about 99% sequence identity thereto.

In some cases, an additional feature of the siAAV is modification of the self-inactivating segment sequences to introduce mismatches relative to the targeting sequence of the gRNA. In some embodiments, the one or more self-inactivating segments each have between 1 to 5 bases different to corresponding positions in the targeting sequence of the first gRNA such that the self-inactivating segments exhibit less efficient cleavage or rate of cleavage by the RNP compared to the cleavage or rate of cleavage of the target nucleic acid. In the foregoing, the base differences of the one or more self-inactivating segments correspond to positions that are 3′ to the fourth nucleotide of the targeting sequence of the first gRNA when the two sequences are aligned. In the embodiments of the foregoing design, the one or more self-inactivating segments can be located within the transgene polynucleotide at the locations previously described, and the CRISPR nuclease, the gRNA, and the regulatory and accessory elements incorporated in the transgene can be selected from the embodiments described herein.

In another design embodiment, the disclosure provides siAAV comprising an AAV capsid protein and a polynucleotide comprising components selected from: i) a 5′ adeno-associated virus (AAV) inverted terminal repeat (ITR) sequence; ii) a 3′ AAV ITR sequence; iii) a sequence encoding a Class 2 Type V CRISPR protein; iv) a first promoter operably linked to the sequence encoding the Class 2 Type V CRISPR protein; v) a sequence encoding a first guide RNA (gRNA) comprising a targeting sequence that is complementary to a target nucleic acid of a cell to be modified; vi) a second promoter sequence operably linked to the sequence encoding the first gRNA; vii) a sequence encoding a second gRNA having a targeting sequence that is complementary to one or more self-limited segments utilized in the polynucleotide; viii) a third promoter sequence operably linked to the sequence encoding the second gRNA, and ix) one or more self-inactivating segments of the polynucleotide comprising a protospacer adjacent motif (PAM) sequence and a polynucleotide sequence having between 1 to 5 bases different to corresponding positions in the targeting sequence of the first gRNA such that the self-inactivating segments exhibit less efficient cleavage or rate of cleavage by the RNP compared to the cleavage or rate of cleavage of the target nucleic acid. In some cases, an additional feature of the foregoing siAAV design is incorporation of a PAM sequence of the one or more self-inactivating segments that is different from the PAM sequence of the target nucleic acid of the cell to be modified, with the result that the PAM promotes less efficient cleavage or rate of cleavage of the self-inactivating segment by the RNP compared to the PAM sequence 5′ and adjacent to the target nucleic acid of the cell to be modified. As previously described, the selection of the PAM sequence of the self-inactivating segments is based on the PAM sequence of the target nucleic acid sequence to be modified, the preference of the CRISPR nuclease utilized, and the rank-order of the strength of the PAM relative to the foregoing; e.g., if the PAM sequence of the target nucleic acid of the cell to be modified is TTC and the PAM preference of the Class 2 Type V CRISPR protein is TTC, then the PAM sequence of the one or more self-inactivating segments is selected from the group consisting of ATC, CTC, TTT, GTT, and GTC. In some cases, an additional feature of the foregoing siAAV design is use of a sequence of the scaffold of the second gRNA that is different than that of the first gRNA and is less efficient in promoting binding and/or editing of the self-limiting segment by the RNP compared to the binding and editing of the target nucleic acid by an RNP having the first gRNA. In some embodiments of the foregoing, the second guide scaffold comprises a sequence selected from the group consisting of SEQ ID NO: 2101-2238 and 3992-3995 and the first guide scaffold comprises a sequence selected from SEQ ID NOS: 2276-2296 and 4028 corresponding to scaffolds 215 to 235 and scaffold 316. In a particular embodiment of the foregoing, the second guide scaffold comprises the sequence of SEQ ID NO: 2238 and the first guide scaffold comprises the sequence of SEQ ID NO: 2296. In some cases, an additional feature of the foregoing siAAV design is incorporation of a third promoter wherein the sequence is different from the second promoter sequence and is less efficient at initiating transcription of the gRNA compared to the second promoter. In some embodiments, the second and the third promoters are selected from the group consisting of U6, truncated U6, sequence variants of U6, mini U6, 5S, Adenovirus 2 (Ad2) VAI, 7SK, truncated 7SK, sequence variants of 7SK, H1, truncated H1, sequence variants of H1, bidirectional H1, bidirectional U6, bidirectional 7SK, and bidirectional U6. In other embodiments, the second and the third promoters are selected from the group consisting of SEQ ID NOS: 494-513 and 2688-2708 set forth in Table 25, or a sequence having at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical or at least about 99% identical thereto. In the embodiments of the foregoing design, the one or more self-inactivating segments can be located within the transgene polynucleotide at the locations previously described, and the CRISPR nuclease, the gRNA, and the regulatory and accessory elements incorporated in the transgene can be selected from the embodiments described herein.

In another design embodiment, the disclosure provides siAAV comprising an AAV capsid protein and a polynucleotide comprising components selected from: i) a 5′ adeno-associated virus (AAV) inverted terminal repeat (ITR) sequence; ii) a 3′ AAV ITR sequence; iii) a sequence encoding a Class 2 Type V CRISPR protein; iv) a first promoter operably linked to the sequence encoding the Class 2 Type V CRISPR protein; v) a sequence encoding a first guide RNA (gRNA) comprising a targeting sequence that is complementary to a target nucleic acid of a cell to be modified; vi) a second promoter sequence operably linked to the sequence encoding the first gRNA; vii) a sequence encoding a second gRNA having a targeting sequence that is complementary to one or more self-limited segments utilized in the polynucleotide, wherein the sequence of the scaffold of the second gRNA is different than that of the first gRNA and is less efficient in promoting binding and/or editing of the self-limiting segment by the RNP compared to the binding and editing of the target nucleic acid by an RNP having the first gRNA; viii) a third promoter sequence operably linked to the sequence encoding the second gRNA, and ix) one or more self-inactivating segments of the polynucleotide comprising a protospacer adjacent motif (PAM) sequence and a polynucleotide sequence capable of being bound and cleaved by a ribonuclear protein complex (RNP) of the Class 2 Type V CRISPR protein and the second gRNA. In some cases of the foregoing design, the sequence of the scaffold of the second gRNA is identical to that of the first gRNA. In some embodiments of the foregoing, the second guide scaffold comprises a sequence selected from the group consisting of SEQ ID NO: 2101-2238 and 3992-3995 and the first guide scaffold comprises a sequence selected from the group consisting of SEQ ID NOS: 2276-2296 and 4028. In a particular embodiment of the foregoing, the second guide scaffold comprises the sequence of SEQ ID NO: 2238 and the first guide scaffold comprises the sequence of SEQ ID NO: 2296. In some cases, an additional feature of the foregoing siAAV design is incorporation of a PAM sequence of the one or more self-inactivating segments that is different from the PAM sequence of the target nucleic acid of the cell to be modified, with the result, as described previously, that the PAM promotes less efficient cleavage or rate of cleavage of the self-inactivating segment by the RNP compared to the PAM sequence 5′ and adjacent to the target nucleic acid of the cell to be modified. In some cases, an additional feature of the foregoing siAAV design is modification of the self-inactivating segment sequences to introduce mismatches relative to the targeting sequence of the gRNA. In some embodiments, the one or more self-inactivating segments each have between 1 to 5 bases different to corresponding positions in the targeting sequence of the first gRNA such that the self-inactivating segments exhibit less efficient cleavage or rate of cleavage by the RNP compared to the cleavage or rate of cleavage of the target nucleic acid. In the foregoing, the base differences of the one or more self-inactivating segments correspond to positions that are 3′ to the fourth nucleotide of the targeting sequence of the first gRNA when the two sequences are aligned. In some cases, an additional feature of the foregoing siAAV design is incorporation of a third promoter wherein the sequence is different from the second promoter sequence and is less efficient at initiating transcription of the gRNA compared to the second promoter. In the foregoing, the second and the third promoter are selected from the group consisting of U6, mini U6, 5S, Adenovirus 2 (Ad2) VAI, 7SK, H1, bidirectional H1, bidirectional U6, bidirectional 7SK, and bidirectional U6, so long as the choice for the second and the third promoter is dictated by the efficiency of the promoter. In other embodiments, the second and the third promoter are selected from the group consisting of SEQ ID NOS: 494-513 and 2688-2708 set forth in Table 25, or a sequence having at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical or at least about 99% identical thereto. In the embodiments of the foregoing design, the one or more self-inactivating segments can be located within the transgene polynucleotide at the locations previously described, and the CRISPR nuclease, the gRNA, and the regulatory and accessory elements incorporated in the transgene can be selected from the embodiments described herein.

In another design embodiment, the disclosure provides siAAV comprising an AAV capsid protein and a polynucleotide comprising components selected from: i) a 5′ adeno-associated virus (AAV) inverted terminal repeat (ITR) sequence; ii) a 3′ AAV ITR sequence; iii) a sequence encoding a Class 2 Type V CRISPR protein; iv) a first promoter operably linked to the sequence encoding the Class 2 Type V CRISPR protein; v) a sequence encoding a first guide RNA (gRNA) comprising a targeting sequence that is complementary to a target nucleic acid of a cell to be modified; vi) a second promoter sequence operably linked to the sequence encoding the first gRNA; vii) a sequence encoding a second gRNA having a targeting sequence that is complementary to one or more self-limited segments utilized in the polynucleotide; viii) a third promoter sequence operably linked to the sequence encoding the second gRNA wherein the sequence is different from the second promoter sequence and is less efficient at initiating transcription of the gRNA compared to the second promoter, and ix) one or more self-inactivating segments of the polynucleotide comprising a protospacer adjacent motif (PAM) sequence and a polynucleotide sequence capable of being bound and cleaved by a ribonuclear protein complex (RNP) of the Class 2 Type V CRISPR protein and the second gRNA. In some cases, an additional feature of the foregoing siAAV design is incorporation of a PAM sequence of the one or more self-inactivating segments that is different from the PAM sequence of the target nucleic acid of the cell to be modified, with the result, as previously described, that the PAM promotes less efficient cleavage or rate of cleavage of the self-inactivating segment by the RNP compared to the PAM sequence 5′ and adjacent to the target nucleic acid of the cell to be modified. In some cases, an additional feature of the foregoing siAAV design is modification of the self-inactivating segment sequences to introduce 1-5 mismatches in the sequence relative to the targeting sequence of the gRNA, as previously described. In some cases, an additional feature of the foregoing siAAV design is use of a sequence of the scaffold of the second gRNA that is different than that of the first gRNA and is less efficient in promoting binding and/or editing of the self-limiting segment by the RNP compared to the binding and editing of the target nucleic acid by an RNP having the first gRNA. In some embodiments of the foregoing, the second guide scaffold comprises a sequence selected from the group consisting of SEQ ID NO: 2101-2238 and 3992-3995 and the first guide scaffold comprises a sequence selected from SEQ ID NOS: 2276-2296 or 4028. In a particular embodiment of the foregoing, the second guide scaffold comprises the sequence of SEQ ID NO: 2238 and the first guide scaffold comprises the sequence of SEQ ID NO: 2296. In the embodiments of the foregoing design, the one or more self-inactivating segments can be located within the transgene polynucleotide at the locations previously described, and the CRISPR nuclease, the gRNA, and the regulatory and accessory elements incorporated in the transgene can be selected from the embodiments described herein.

The present disclosure provides polynucleotides for production of siAAV transgene plasmids as well as for the production of siAAV viral vectors wherein the SIRV polynucleotide is designed to include one or more self-inactivating segments integrated into the polynucleotide that, depending on additional components incorporated into the polynucleotide, result in diminished or eliminated expression of the CRISPR components. As previously described herein, the polynucleotides encoding the transgenes are specifically designed such that there is a temporal or activity difference between the capacity of the expressed CRISPR nuclease and guide RNA (gRNA), complexed as an RNP, that is capable of binding and cleaving the target nucleic acid of a cell to be modified, compared to the binding and cleaving of the self-inactivating segments utilized in the transgene that results in the self-inactivation of the construct or the reduced expression of one or more of the CRISPR components. In some embodiments, the temporal-limited expression of the CRISPR components of the siAAV; e.g., CasX and/or gRNA, is designed to reduce or eliminate unwanted off-target effects of the endonuclease activity. In other embodiments, the temporal control of the CRISPR nuclease expression imparted by the designs described herein similarly serve to lower or preclude host immune responses to the nuclease, resulting in enhanced safety and an increased therapeutic ratio of the administered composition.

The various designs of the siAAV constructs and the rationale for the mechanisms by which these differential activities are achieved were described herein, supra. The disclosure also contemplates use of different viral systems incorporating the SIRV designs in which the approaches and components are similar, if not identical, to those utilized for the siAAV, but which comprise packaging components particular to the virus system employed. For adenovirus, a key protein in the initiation of packaging is IVa2 (Ahi, Y. S., et al. Components of Adenovirus Genome Packaging. Front Microbiol. 7:1503 (2016)). In the case of lentivirus and gamma retrovirus, the key component for packaging is referred to as Ψ (psi) (Kuzembayeva, M., et al. Life of psi: How full-length HIV-1 RNAs become packaged genomes in the viral particles. Virology 454:362 (2014); Bolche, J. T., et al. Viral vector platforms within the gene therapy landscape. Signal Transduction Targeted Ther. 6: 53 (2021)).

### VII. Systems and Methods for Modification of Target Nucleic Acids

The SIRV and siAAV provided herein are useful for various applications, including therapeutics, diagnostics, and for research. To effect the methods of the disclosure for gene editing or modification, provided herein are programmable systems that are designed to edit target nucleic acid of a cell and then to self-inactivate. The programmable nature of the Class 2 Type V protein, CasX and gRNA components of the systems provided herein allows for the precise targeting to achieve the desired effect (nicking, cleaving, etc.) at one or more regions of predetermined interest in the target nucleic acid sequence. In some embodiments, the SIRV and siAAV systems provided herein comprise sequences encoding a CasX protein and a gRNA wherein the targeting sequence of the gRNA is complementary to, and therefore is capable of hybridizing with, a target nucleic acid sequence. In some cases, the SIRV and siAAV system further comprises a donor template nucleic acid. The SIRV and siAAV constructs further comprise one or more self-inactivating segments that, when cleaved by an RNP of the CRISPR nuclease and gRNA, reduce or eliminate further expression of the CRISPR components, enhancing the safety of the resulting SIRV and siAAV and reducing the potential for eliciting an immune response to the CRISPR protein.

In some embodiments of the disclosure, provided herein are methods of modifying a target nucleic acid sequence utilizing the SIRV or siAAV compositions of the disclosure. In some embodiments, the methods comprise transfecting or transducing a cell comprising the target nucleic acid sequence with an SIRV or siAAV encoding a Class 2 Type V protein, e.g, a CasX protein of the disclosure and a gRNA of the disclosure comprising a targeting sequence, wherein the targeting sequence of the gRNA has a sequence complementary to and that can hybridize with the sequence of the target nucleic acid. Upon hybridization with the target nucleic acid by the expressed CasX and the gRNA, the CasX introduces one or more single-strand breaks or double-strand breaks within or near the target nucleic acid, which may include sequences that contain regulatory elements, coding regions, or non-coding regions of the gene, that results in a permanent indel (deletion or insertion) or mutation in the target nucleic acid, as described herein, with a corresponding modulation of expression or alteration in the function of the gene product, thereby creating an edited cell. The edits can be effected by the cell's repair mechanisms, such as non-homologous end joining (NHEJ), homology-directed repair (HDR), homology-independent targeted integration (HITI), micro-homology mediated end joining (MMEJ), single strand annealing (SSA) or base excision repair (BER).

In other embodiments, the method comprises contacting a cell comprising the target nucleic acid sequence with an SIRV or siAAV encoding a plurality of gRNAs (i.e., two or more) targeted to different or overlapping portions of the target nucleic acid wherein the Class 2 Type V protein, e.g, a CasX protein introduces multiple breaks in the target nucleic acid that result in a permanent indel or mutation in the target nucleic acid, as described herein, with a corresponding modulation of expression or alteration in the function of the gene product, thereby creating an edited cell. In some embodiments, the modification of the target nucleic acid results in reduced expression of a gene product of a gene comprising the target nucleic acid, wherein expression is reduced by at least about 10%, at least about 20%, at least about 30%, at least about 40%, at least about 50%, at least about 60%, at least about 70%, at least about 80%, or at least about 90% in comparison to a cell that has not been modified. In some embodiments, the modification of the target nucleic acid results in a correction or compensation for a mutation in the gene comprising the target nucleic acid such that such that functional protein (or the gene product) is expressed by the modified cells. In some embodiments of the method, expression of the functional protein by the cells of the population is increased by at least about 10%, at least about 20%, at least about 30%, at least about 40%, at least about 50%, at least about 60%, at least about 70%, at least about 80%, or at least about 90% in comparison to a cell where the gene has not been modified. As disclosed, supra, the SIRV and siAAV polynucleotide configurations comprising the self-inactivating segments are designed to permit the editing or modification of the target nucleic acid expression of the CRISPR nuclease and guide to occur, with the less efficient self-inactivating mechanisms permitting a temporal difference between the desired editing or modification and the cleavage of the target nucleic acid and the cleavage of the self-inactivating segment polynucleotide; the latter resulting in decreased or elimination of transcription of the CRISPR components of the SIRV polynucleotide. Examples of such designs exhibiting editing or modification and subsequent inactivation are described herein, in the Examples.

In some embodiments of the method of modifying a target nucleic acid sequence using the SIRV and siAAV compositions of the embodiments, the encoded Class 2 Type V protein, CasX protein is a reference CasX selected from SEQ ID NOS: 1-3, or a CasX variant having at least 50%, at least 60%, at least 70%, at least 80%, or at least 90%, or at least 95%, or at least 99% sequence identity to the reference CasX proteins of SEQ ID NOS:1-3; embodiments of which are more fully described, supra. In some embodiments of the method, the SIRV encodes a Class 2 Type V protein, CasX variant having a sequence of any one of the sequences of SEQ ID NOS: 49-321 and 2356-2488, or a sequence having at least about 50%, at least about 60%, at least about 70%, at least about 80%, at least about 90%, or at least about 95%, or at least about 96%, or at least about 97%, or at least about 98%, or at least about 99% sequence identity thereto wherein the CasX variant protein exhibits at least one or more improved characteristics as compared to a reference CasX protein of SEQ ID NOS:1-3, and the gRNA scaffold comprises any one of the sequences of SEQ ID NOS: 2101-2331, 3992-3995, and 4028, as set forth in Table 2. In some embodiments of the method, the one or more improved characteristics of the CasX variant protein and gRNA variant are selected from the group consisting of improved folding of the CasX protein, improved binding affinity to the guide RNA, improved binding affinity to the target nucleic acid sequence, altered binding affinity to one or more PAM sequences, ability to effectively bind a greater spectrum of PAM sequences of a nucleic acid compared to reference CasX proteins, including TTC, ATC, GTC, and CTC, improved unwinding of the target nucleic acid sequence, increased activity, improved editing efficiency, improved editing specificity, increased activity of the nuclease, increased target strand loading for double strand cleavage, decreased target strand loading for single strand nicking, decreased off-target cleavage, improved binding of the non-target strand of DNA, improved protein stability, improved protein:guide RNA complex stability, improved protein solubility, improved protein:guide RNA complex solubility, improved protein expression, and improved fusion characteristics. In some embodiments of the methods, the improved characteristic of the CasX variant protein is at least about 1.1 to about 100,000-fold improved relative to the reference protein of SEQ ID NO: 1, SEQ ID NO: 2, SEQ ID NO: 3 and the reference gRNA of SEQ ID NOS: 4-16. In some embodiments, the improved characteristic of the CasX variant protein and gRNA variant is at least about 1.1, at least 1.5, at least 10, at least 50, at least 100, at least 500, at least 1,000, at least 5,000, or at least a 10,000-fold improved, as compared to a reference CasX protein of SEQ ID NO: 1, SEQ ID NO: 2, SEQ ID NO: 3 and the reference gRNA of SEQ ID NOS: 4-16.

In some embodiments of the method, the modifying of the target nucleic acid sequence is carried out in vitro inside a cell. In some embodiments, the cell is a eukaryotic cell selected from the group consisting of a rodent cell, a mouse cell, a rat cell, a primate cell, and a non-human primate cell. In particular embodiments, the eukaryotic cell is a human cell. In some embodiments of the method, the modifying of the target nucleic acid sequence is carried out in vivo in a subject. In some embodiments, the subject is selected from the group consisting of mouse, rat, pig, and non-human primate. In another embodiment, the subject is human.

In some embodiments, the method of modifying a target nucleic acid sequence comprises contacting a target nucleic acid of a cell with an SIRV or siAAV vector encoding a CasX protein, one or two gRNA, and further comprising a donor template. The donor template may be inserted into the target nucleic acid such that all, some or none of the gene product is expressed. Depending on whether the system is used to knock-down/knock-out or to knock-in a protein-coding sequence, the donor template can be a short single-stranded or double-stranded oligonucleotide, or can be a long single-stranded or double-stranded oligonucleotide. For knock-down/knock-outs, the donor template sequence need not be identical to the genomic sequence that it replaces and may contain one or more single base changes, insertions, deletions, inversions or rearrangements with respect to the genomic sequence such that expression of the gene product is reduced by at least about 10%, at least about 20%, at least about 30%, at least about 40%, at least about 50%, at least about 60%, at least about 70%, at least about 80%, or at least about 90% in comparison to target nucleic acid that has not been modified. Provided that there are arms with sufficient numbers of nucleotides having sufficient homology flanking the cleavage site(s) of the target nucleic acid sequence targeted by the CasX:gRNA (i.e., 5′ and 3′ to the cleavage site) to support homology-directed repair (“homologous arms”), use of such donor templates can result in a frame-shift or other mutation such that the gene product is not expressed or is expressed at a lower level. In some embodiments, the homologous arms comprise between 10 and 100 nucleotides, facilitating insertion of the donor template sequence by HDR. In other cases, an exogenous donor template may comprise a corrective sequence to be integrated and is flanked by an upstream homologous arm and a downstream homologous arm, each having homology to the target nucleic acid sequence that is introduced into a cell resulting in expression of functional gene product.

Introducing recombinant SIRV and siAAV vectors comprising sequences encoding the transgene components (e.g., the self-inactivating segments, CasX, gRNA, promoters and accessory components and, optionally, the donor template sequences) of the disclosure into cells under in vitro conditions can occur in any suitable culture media and under any suitable culture conditions that promote the survival of the cells and production of the CasX:gRNA. Introducing recombinant SIRV or siAAV vectors into a target cell can be carried out in vivo, in vitro or ex vivo. In some embodiments of the method, vectors may be provided directly to a target host cell such that the vectors are taken up by the cells.

VIII. Methods of Making siAAV Vectors

In other aspects, the disclosure relates to methods to produce the siAAV vectors of any of the embodiments described herein, as well as methods to express and recover the siAAV. In general, the methods include producing a polynucleotide sequence coding for the components of the expression cassette plus the flanking ITRs of any of the embodiments described herein and incorporating the encoding gene into an expression vector appropriate for a host cell. For production of the siAAV vector of any of the embodiments described herein, the methods include transforming an appropriate host cell using a two or three plasmid system with an expression vector comprising the transgene polynucleotide encoding the CRISPR components, together with a pRC plasmid comprising the Rep and Cap sequences provided in trans and the pHelper plasmid (containing essential genes such as E2A, E4 and VA), and culturing the transformed packaging host cell under conditions causing or permitting the resulting siAAV to be produced, which are recovered by methods described herein or by standard purification methods known in the art. Alternatively, the host cell genome may comprise stably integrated Rep and Cap (1 or 2) and helper genes. Suitable packaging cell lines are known to one of ordinary skill in the art, including, but not limited to HEK293, HEK293T, HeLa or A549. See for example, www.cellbiolabs.com/aav-expression-and-packaging. Methods of purifying siAAV produced by host cell lines will be known to one of ordinary skill in the art, and include, without limitation, affinity chromatography, gradient centrifugation, and ion exchange chromatography.

Standard recombinant techniques in molecular biology are used, along with the methods of the Examples, to make the polynucleotides and SIRV and siAAV vectors of the present disclosure. In accordance with the methods of the disclosure, nucleic acid sequences that encode the self-inactivating segment, reference CasX, the CasX variants, or the gRNA of any of the embodiments described herein are used to generate recombinant DNA molecules that direct the expression in appropriate host cells. Several cloning strategies are suitable for performing the methods of the present disclosure, many of which are used to generate a construct that comprises a gene coding for a composition of the present disclosure, or its complement. In some approaches, a construct is first prepared containing the DNA sequences encoding the components of the siAAV vector and transgene. Exemplary methods for the preparation of such constructs are described in the Examples. The nucleic acid sequences encoding the transgene components are inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan. Such techniques are well known in the art and well described in the scientific and patent literature. Various vectors are publicly available. The construct is then used to create an expression vector suitable for transforming a host packaging cell, such as a eukaryotic host cell for the expression and recovery of the siAAV vector comprising the transgene. The eukaryotic host cell can be selected from BHK cells, HEK293 cells, HEK293T cells, NS0 cells, SP2/0 cells, YO myeloma cells, A549 cells, P3X63 mouse myeloma cells, PER cells, PER.C6 cells, hybridoma cells, NIH3T3 cells, COS, HeLa, CHO, or other eukaryotic cells known in the art suitable for the production of recombinant siAAV. A number of transfection techniques are generally known in the art; see, e.g., Sambrook et al. (1989) Molecular Cloning, a laboratory manual, Cold Spring Harbor Laboratories, New York. Particularly suitable transfection methods include calcium phosphate co-precipitation, direct microinjection into cultured cells, electroporation, liposome mediated gene transfer, lipid-mediated transduction, and nucleic acid delivery using high-velocity microprojectiles. Exemplary methods for the creation of expression vectors, the transformation of host cells and the expression and recovery of the nucleic acids and the siAAV vectors are described in the Examples.

The gene encoding the siAAV vector can be made in one or more steps, either fully synthetically or by synthesis combined with enzymatic processes, such as restriction enzyme-mediated cloning, PCR and overlap extension, including methods more fully described in the Examples. The methods disclosed herein can be used, for example, to ligate sequences of polynucleotides encoding the various components (e.g., self-limiting segments, ITRs, Class 2 Type V protein, e.g., a CasX, and gRNA, promoters and accessory elements) of a desired sequence to create the expression vector.

In some embodiments, host cells transduced with the above-described siAAV expression vectors are rendered capable of providing AAV helper functions in order to replicate and encapsidate the nucleotide sequences flanked by the AAV ITRs to produce siAAV viral particles. AAV helper functions are generally AAV-derived coding sequences which can be expressed to provide AAV gene products that, in turn, function in trans for productive AAV replication. AAV helper functions are used herein to complement necessary AAV functions that are missing from the AAV expression vectors. Thus, AAV helper functions include one, or both of the major AAV ORFs (open reading frames), encoding the rep and cap coding regions, or functional homologues thereof. Accessory functions can be introduced into and then expressed in host cells using methods known to those of skill in the art. Commonly, accessory functions are provided by infection of the host cells with an unrelated helper virus. In some embodiments, accessory functions are provided using an accessory function vector. Depending on the host/vector system utilized, any of a number of suitable transcription and translation control elements, including constitutive and inducible promoters, transcription enhancer elements, transcription terminators, etc., may be used in the expression vector.

In some embodiments, the nucleotide sequence encoding the CRISPR protein components of the siAAV vector is codon optimized. This type of optimization can entail a mutation of an encoding nucleotide sequence to mimic the codon preferences of the intended host organism or cell while encoding the same CRISPR protein or other protein component. Thus, the codons can be changed, but the encoded protein remains unchanged. For example, if the intended host cell was a human cell, a human codon-optimized encoding nucleotide sequence could be used. The gene design can be performed using algorithms that optimize codon usage and amino acid composition appropriate for the host cell utilized in the production of the siAAV vector. In one method of the disclosure, a library of polynucleotides encoding the components of the constructs is created and then assembled, as described above. The resulting genes are then assembled and the resulting genes used to transform a host cell and produce and recover the siAAV vector compositions for evaluation of its properties, as described herein.

In some embodiments, the present disclosure provides siAAV vectors in which the CpG motifs of the polynucleotide of the siAAV are reduced or eliminated. By reducing or eliminating the CpG motifs, the immunogenicity of the siAAV is reduced, while retaining their functional characteristics. In particular, CpG dinucleotide motifs (CpG PAMPs) in AAV vectors are immunostimulatory because of their high degree of hypomethylation, relative to mammalian CpG motifs, which have a high degree of methylation. Accordingly, reducing the frequency of unmethylated CpGs in rAAV vector genomes to a level below the threshold that activates human TLR9 is expected to reduce the immune response to exogenously administered AAV-based biologics. In some embodiments, the CpG motifs are reduced or eliminated in the nucleic acid sequences of one or more components of the siAAV selected from the group consisting 5′ ITR, 3′ ITR, Pol III promoter, Pol II promoter, encoding sequence for CRISPR nuclease, encoding sequence for gRNA, accessory element, and poly(A) signal. In some embodiments, the present disclosure provides rAAV vectors wherein one or more components of the transgene are codon-optimized for depletion of CpG dinucleotides by the substitution of homologous nucleotide sequences from mammalian species, wherein the one or more components substantially retain their functional properties upon expression in a transduced cell; e.g., ability to drive expression of the CRISPR nuclease, ability to drive expression of the gRNA, enhance the expression of the CRISPR nuclease and/or the gRNA, and enhanced ability to edit a target nucleic acid sequence. In some embodiments, the present disclosure provides siAAV vectors wherein the transgene comprises less than about 10%, less than about 5%, or less than about 1% CpG dinucleotides. In some embodiments, the present disclosure provides siAAV vectors wherein the one or more siAAV component sequences codon-optimized for depletion of CpG dinucleotides are selected from the group of sequences consisting of SEQ ID NOS: 2904-2915, 2917-2919, 4021-4027, and 4029-4050, or a sequence having at least about 80%, at least about 90%, at least about 95%, at least about 96%, at least about 97%, at least about 98%, or at least about 99% sequence identity thereto.

The siAAV vectors used for providing the nucleic acids encoding gRNAs and the CRISPR proteins, as well as the self-inactivating segments, to a target host cell can include regulatory elements such as suitable promoters or other accessory elements for driving the expression, that is, transcriptional activation of the nucleic acid of interest. In some cases, the encoding nucleic acid of interest will be operably linked to a promoter. In some embodiments, each component (i.e., the CasX and the one or more gRNA) will have a linked promoter chosen to optimize or tailor the transcription of the encoding nucleic acid. This may include ubiquitously acting promoters, for example, the CMV-beta-actin promoter, or inducible promoters, such as promoters that are active in particular cell populations or that respond to the presence of drugs such as tetracycline or kanamycin. By transcriptional activation, it is intended that transcription will be increased above basal levels in the target host cell comprising the vector by at least about 10-fold, by at least about 100-fold, more usually by at least about 1000-fold. In addition, vectors used for providing a nucleic acid encoding a gRNA and/or a CasX protein to a cell may include nucleic acid sequences that encode for selectable markers in the target cells, so as to identify cells that have taken up the CasX protein and/or the gRNA.

In some cases, the promoter is a constitutively active promoter. In some cases, the promoter is a regulatable promoter. In some cases, the promoter is an inducible promoter. In some cases, the promoter is a tissue-specific promoter.

In some embodiments, the promoter is a bidirectional promoter able to control initiation of transcription of two encoded components of the SIRV construct; e.g., two guide RNAs or a gRNA and a CRISPR nuclease. Bidirectional promoters are known in the art (WO2005/035718 and PCT/US2004/032158, incorporated by reference herein), as well as those described in the Examples.

Non-limiting examples of promoters functional in the siAAV constructs include EF-1alpha, EF-1alpha core promoter, Jens Tornoe (JeT), promoters from cytomegalovirus (CMV), CMV immediate early (CMVIE), CMV enhancer, herpes simplex virus (HSV) thymidine kinase, early and late simian virus 40 (SV40), the SV40 enhancer, long terminal repeats (LTRs) from retrovirus, mouse metallothionein-I, adenovirus major late promoter (Ad MLP), CMV promoter full-length promoter, the minimal CMV promoter, the chicken 3-actin promoter (CBA), CBA hybrid (CBh), chicken β-actin promoter with cytomegalovirus enhancer (CB7), chicken beta-Actin promoter and rabbit beta-Globin splice acceptor site fusion (CAG), the rous sarcoma virus (RSV) promoter, the HIV-Ltr promoter, the hPGK promoter, the HSV TK promoter, a 7SK promoter, the Mini-TK promoter, the human synapsin I (SYN) promoter which confers neuron-specific expression, beta-actin promoter, super core promoter 1 (SCP1), the Mecp2 promoter for selective expression in neurons, the minimal IL-2 promoter, the Rous sarcoma virus enhancer/promoter (single), the spleen focus-forming virus long terminal repeat (LTR) promoter, the TBG promoter, promoter from the human thyroxine-binding globulin gene (Liver specific), the PGK promoter, the human ubiquitin C promoter (UBC), the UCOE promoter (Promoter of HNRPA2B1-CBX3), the synthetic CAG promoter, the Histone H2 promoter, the Histone H3 promoter, the U1a1 small nuclear RNA promoter (226 nt), the U1a1 small nuclear RNA promoter (226 nt), the Ulb2 small nuclear RNA promoter (246 nt) 26, the GUSB promoter, the CBh promoter, rhodopsin (Rho) promoter, silencing-prone spleen focus forming virus (SFFV) promoter, a human H1 promoter (H1), a POL1 promoter, the TTR minimal enhancer/promoter, the b-kinesin promoter, mouse mammary tumor virus long terminal repeat (LTR) promoter, the human eukaryotic initiation factor 4A (EIF4A1) promoter, the ROSA26 promoter, the glyceraldehyde 3-phosphate dehydrogenase (GAPDH) promoter, tRNA promoters, and truncated versions and sequence variants of the foregoing. In some embodiments, the promoters used to promote transcription of the gRNA include U6 (Kunkel, G R et al. U6 small nuclear RNA is transcribed by RNA polymerase III. Proc Natl Acad Sci USA. 83(22):8575 (1986)), U6 truncated promoters, U6 bidirectional promoters, mini U6 promoters, 5S promoter, Adenovirus 2 (Ad2) VAI promoter, 7SK promoter, H1 promoter, bidirectional H1 promoter, bidirectional 7SK promoter, bidirectional U6 promoter, and sequence variants thereof. Exemplary sequences are presented as SEQ ID NOS: 425-431, 463-513, and 2688-2708 as set forth in Tables 8, 10, 11, and 25.

Selection of the appropriate promoter is well within the level of ordinary skill in the art, as it relates to controlling expression, e.g., for modifying the SIRV transgene or the target nucleic acid. The expression vector may also contain a ribosome binding site for translation initiation and a transcription terminator. The expression vector may also include appropriate sequences for amplifying expression. The expression vector may also include nucleotide sequences encoding protein tags (e.g., 6×His tag, hemagglutinin tag, fluorescent protein, etc.) that can be fused to the CasX protein, thus resulting in a chimeric CasX protein that are used for purification or detection.

In a feature of the siAAV design, promoters and regulatory elements incorporated into the transgene for the control of transcription of the individual components are chosen to achieve differential levels of transcription activation in order to obtain the desired functional outcome for the expressed component. In the case of constructs incorporating shRNA to control translation of the CRISPR protein in packaging cells (discussed more fully, below), a strong promoter, such as EF-1α, native U6, H1, and 7SK or other pol III or pol II promoters known in the art to be strong, can be utilized to ensure that the shRNA is transcribed in sufficient quantities to knock-down translation of the CRISPR protein.

In the case of constructs intended for specific tissues, tissue-specific promoters and enhancers can be utilized. Non-limiting examples of tissue-specific promoters are provided as SEQ ID NOS: 425-431 in Table 8. In some cases, the promoter is a cell type-specific promoter. In some cases, the promoter is functional in a targeted cell type or targeted cell population.

TABLE 8
Tissue-specific promoters

| Name | SEQ ID NO: |
|---|---|
| p69.hRHO536 | 425 |
| p69.hRHO 536-SV40. | 426 |
| p69.hRHO536-CAG(no RBI). | 427 |
| p69.hGRK93-SV40. | 428 |
| p69.MeP426 SV40 NLS | 429 |
| p69.MeP426 cmyc NLS | 430 |
| p69.Mecp2 | 431 |

In some embodiments, promoters for siAAV constructs intended for use in muscle can include Desmin, CK8e, MHCK7, or MHCK to control translation of the CRISPR protein. In some embodiments, enhancers for siAAV constructs intended for use in muscle can include a sequence selected from the group consisting of SEQ ID NOS: 3779-3809. In some embodiments, promoters for siAAV constructs intended for use in the eye can include RHO, RH0535-CAG, RHO-intron, endogenous G-coupled Rhodopsin Kinase 1 (GRK1), GRK1-SV40, or GRK1-CAG. In the case of siAAV designs incorporating a second gRNA that targets the self-inactivating segments, a weaker promoter can be utilized such that the transcription of the second gRNA is delayed or reduced in comparison to the expression of the CRISPR protein and first guide intended for gene editing, such that inactivation of CRISPR components does not occur prematurely before the desired gene editing has occurred. A non-limiting example of a weaker promoter is a truncated U6 promoter or a sequence variant of a U6 promoter. In an alternative approach, in some embodiments the siAAV comprises a second gRNA that targets the self-inactivating segments wherein the gRNA is less efficient at promoting cleavage when incorporated into an RNP compared to the first gRNA of the construct. A non-limiting example of a less efficient gRNA is gRNA 64 (SEQ ID NO: 2106) compared to gRNA 174 (SEQ ID NO: 2238) or gRNA 225 (SEQ ID NO: 2286), or gRNA 174 (SEQ ID NO: 2238) compared to gRNA 225 (SEQ ID NO: 2286). In such designs, the cleavage of the self-inactivating segment is delayed or reduced, thereby increasing the ability of the CRISPR protein and first guide to effect the intended gene editing of the target nucleic acid prior to the cleavage of the self-inactivating segment.

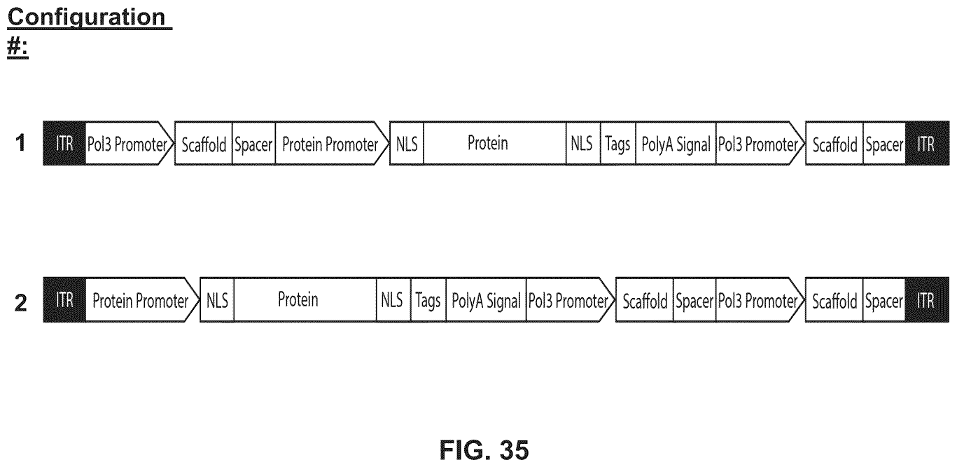

In some embodiments, the disclosure provides siAAV constructs in which the promoters of the transgene driving the expression of the gRNA are placed in either in the forward or reverse orientation (see, e.g., FIGS. 35, 36, 104 and 112) in order to control modulation of expression in siAAV constructs that contain one or multiple guides. It will be understood that in such cases where the promoter is placed in a reverse orientation, the gRNA are expressed from the antisense strand of the episome, while promoter in a forward orientation would express the gRNA from the sense strand.

Exemplary accessory elements for inclusion in the polynucleotide of the siAAV construct include a transcription enhancer element, a transcription termination signal, internal ribosome entry site (IRES) or P2A peptide to permit translation of multiple genes from a single transcript, polyadenylation sequences to promote downstream transcriptional termination, sequences for optimization of initiation of translation, and translation termination sequences. In some embodiments, the accessory element is selected from the group consisting of a poly(A) signal, a gene enhancer element, an intron, a posttranscriptional regulatory element (PTRE), a deaminase, a DNA glycosylase inhibitor, a stimulator of CRISPR-mediated homology-directed repair, an activator or repressor of transcription, and a self-cleaving sequence. In some embodiments, the PTRE is selected from the group consisting of cytomegalovirus immediate/early intronA, hepatitis B virus PRE (HPRE), Woodchuck Hepatitis virus PRE (WPRE), and 5′ untranslated segment (UTR) of human heat shock protein 70 mRNA (Hsp70). Representative, non-limiting examples of promoters and accessory element sequences suitable for incorporation into the siAAV constructs of the disclosure include the promoters of Tables 8, 10, 11, 25, 54-55, and 57-58, the poly(A) signal sequences of Tables 12 and 15, and SEQ ID NOS: 2991-3991, the PTRE of Table 18, enhancers linked to core promoters of Table 16, the NLS of Tables 7, 22, and 23, and the introns of Table 24.

The recombinant expression vectors of the disclosure can also comprise elements that facilitate robust expression or repress expression of the siAAV transgene components of the disclosure (e.g., the self-inactivating segments, Class 2 Type V protein, e.g, a CasX, or the gRNA). For example, recombinant expression vectors can include one or more of a polyadenylation (poly(A)) signal, an intronic sequence or a post-transcriptional accessory element such as a woodchuck hepatitis post-transcriptional regulatory element (PTRE). Exemplary poly(A) signal sequences include hGH poly(A) signal (short), HSV TK poly(A) signal, synthetic polyadenylation signals, SV40 poly(A) signal, β-globin poly(A) signal and the like, including the sequences of SEQ ID NOS: 514-523 as set forth in Table 12, SEQ ID NOS: 2710-2859, and SEQ ID NOS: 2991-3991. In some embodiments, the vectors of the disclosure comprise one or more sequences comprising PTRE selected from the group consisting of SEQ ID NOS: 524-526 set forth in Table 18. A person of ordinary skill in the art will be able to select suitable elements to include in the recombinant expression vectors described herein.

In some embodiments of the method, host cells transduced with the above-described siAAV expression vectors are rendered capable of providing AAV helper functions in order to replicate and encapsidate the nucleotide sequences flanked by the AAV ITRs to produce rAAV viral particles. AAV helper functions are generally AAV-derived coding sequences which can be expressed to provide AAV gene products that, in turn, function in trans for productive AAV replication. AAV helper functions are used herein to complement necessary AAV functions that are missing from the AAV expression vectors. Thus, AAV helper functions include one, or both of the major AAV ORFs (open reading frames) encoding the rep and cap coding regions, or functional homologues thereof. Accessory functions can be introduced into and then expressed in host cells using methods known to those of skill in the art. Commonly, accessory functions are provided by infection of the host cells with an unrelated helper virus. In some embodiments, accessory functions are provided using an accessory function vector. Depending on the host/vector system utilized, any of a number of suitable transcription and translation accessory elements, including constitutive and inducible promoters, transcription enhancer elements, transcription terminators, etc., may be used in the expression vector.

### IX. Therapeutic Methods

The present disclosure provides methods of treating a disease in a subject in need thereof. In some embodiments, the methods of the disclosure can prevent, treat and/or ameliorate a genetic disease of a subject by the administering to the subject of an siAAV composition of the disclosure. In some embodiments, the composition administered to the subject further comprises a pharmaceutically acceptable carrier, diluent or excipient.

In some embodiments, the disclosure provides methods of treating a disease in a subject having a mutation or a sequence that results in a cellular or physiologic abnormality in cells of the subject, the modifying comprising administering to the subject a therapeutically effective dose of an siAAV vector of any of the embodiments described herein wherein the targeting sequence of the encoded gRNA has a sequence that hybridizes with the target nucleic acid, resulting in the modification of the target nucleic acid by the CasX protein. In some embodiments of the method, the modified target nucleic acid comprises a single-stranded break, resulting in a mutation, an insertion, or a deletion effected by the repair mechanisms of the cell. In other embodiments of the method, the modified target nucleic acid comprises a double-stranded break, resulting in a mutation, an insertion, or a deletion effected by the repair mechanisms of the cell. For example, the expressed CasX:gRNA RNP can introduce into the cell an indel; e.g., a frameshift mutation, at or near the initiation point of the gene.

In other embodiments, the method of treatment comprises administering to the subject a therapeutically effective dose of an siAAV vector encoding a plurality (e.g., two or more) of gRNAs targeted to different or overlapping regions of the target nucleic acid with one or more mutations or duplications. In the foregoing, the resulting modification can be an insertion, deletion, substitution, duplication, or inversion of one or more nucleotides as compared to the target nucleic acid sequence.

In other embodiments, the methods of treating a disease in a subject in need thereof comprise administering to the subject a therapeutically effective dose of an siAAV vector of any of the embodiments described herein wherein the targeting sequence of the encoded gRNA has a sequence that hybridizes with the target nucleic acid and wherein the siAAV further comprises a donor template comprises one or more mutations or a heterologous sequence that is inserted into or replaces the target nucleic acid sequence to knock-down or knock-out the gene comprising the target nucleic acid. In the foregoing, the insertion of the donor template serves to disrupt expression of the gene and the resulting gene product. In some embodiments of the foregoing methods, the donor DNA template ranges in size from 10-1,000 nucleotides. In other embodiments of the foregoing methods, the donor template ranges in size from 100-500 nucleotides. In some cases, the donor template is a single-stranded RNA or DNA template.

In other embodiments, the methods of treating a disease in a subject in need thereof comprise administering to the subject a therapeutically effective dose of a lipid nanoparticle (LNP) comprising the SIRV of any of the embodiments described herein wherein the targeting sequence of the encoded gRNA has a sequence that hybridizes with the target nucleic acid resulting in the modification of the target nucleic acid by the CasX protein.

The modified cells of the treated subject can be a eukaryotic cell selected from the group consisting of a rodent cell, a mouse cell, a rat cell, a primate cell, and a non-human primate cell. In some embodiments, the eukaryotic cells of the treated subject is a human cell.

In some embodiments, the method comprises administering to the subject the siAAV vector of the embodiments described herein via an administration route selected from the group consisting of subcutaneous, intradermal, intraneural, intranodal, intramedullary, intramuscular, intralumbar, intrathecal, subarachnoid, intraventricular, intracapsular, intravenous, intraarterial, intralymphatical, intraocular or intraperitoneal routes, wherein the administering method is injection, transfusion, or implantation. In some embodiments of the methods of treating a disease in a subject, the subject is selected from the group consisting of mouse, rat, pig, non-human primate, and human. In a particular embodiment, the subject is a human.

In some embodiments of the method of treatment, the siAAV vector is administered at a dose of at least about 1×10⁵ vector genomes/kg (vg), at least about 1×10⁶ vg/kg, at least about 1×10⁷ vg/kg, at least about 1×10⁸ vg/kg, at least about 1×10⁹ vg/kg, at least about 1×10¹⁰ vg/kg, at least about 1×10¹¹ vg/kg, at least about 1×10¹² vg/kg, at least about 1×10¹³ vg/kg, at least about 1×10¹⁴ vg/kg, at least about 1×10¹⁵ vg/kg, at least about 1×10⁶ vg/kg. In other embodiments of the method of treatment, the siAAV vector is administered to a subject at a dose of at least about 1×10⁵ vg/kg to about 1×10¹⁶ vg/kg, at least about 1×10⁶ vg/kg to about 1×10¹⁵ vg/kg, or at least about 1×10⁷ vg/kg to about 1×10¹⁴ vg/kg.

A number of therapeutic strategies have been used to design the compositions for use in the methods of treatment of a subject with a disease. In some embodiments, the invention provides a method of treatment of a subject having a disease, the method comprising administering to the subject an siAAV vector of any of the embodiments disclosed herein according to a treatment regimen comprising one or more consecutive doses using a therapeutically effective dose. In some embodiments of the treatment regimen, the therapeutically effective dose of the siAAV vector is administered as a single dose. In other embodiments of the treatment regimen, the therapeutically effective dose is administered to the subject as two or more doses over a period of at least two weeks, or at least one month, or at least two months, or at least three months, or at least four months, or at least five months, or at least six months. In some embodiments of the treatment regiment, the effective doses are administered by a route selected from the group consisting of subcutaneous, intradermal, intraneural, intranodal, intramedullary, intramuscular, intralumbar, intrathecal, subarachnoid, intraventricular, intracapsular, intravenous, intraarterial, intralymphatical, intraocular, subretinal, intravitreal, or intraperitoneal routes, wherein the administering method is injection, transfusion, or implantation.

In some embodiments, the administering of the therapeutically effective amount of an siAAV vector to knock down or knock out expression of a gene having one or more mutations leads to the prevention or amelioration of the underlying disease such that an improvement is observed in the subject, notwithstanding that the subject may still be afflicted with the underlying disease. In some embodiments, the administration of the therapeutically effective amount of the siAAV vector leads to an improvement in at least one clinically-relevant parameter for the disease. In some embodiments of the method of treatment, the subject is selected from mouse, rat, pig, dog, and non-human primate. In a particular embodiment, the subject is human.

X. Controlled Production of siAAV Utilizing shRNA

In another aspect, the present disclosure relates to polynucleotide constructs specifically designed to produce siAAV in eukaryotic packaging cells transfected with the plasmids encoding the siAAV while repressing the degradation of the CRISPR nuclease that would otherwise occur due to the self-inactivating features of the constructs.

In some embodiments, the disclosure provides systems of polynucleotides comprising sequences encoding short-hairpin RNA (shRNA) that are processed by the eukaryotic packaging cells into small interfering RNA (siRNA) that are complementary to portions of the Class 2, Type V CRISPR mRNA expressed by the packaging cells, wherein the intracellular processing mechanisms of the cell result in the cleavage of the mRNA such that the expression by translation of the CRISPR protein is repressed. As used herein, the term “repressed” or “repression” includes partial reduction or complete extinction or silencing of expression.

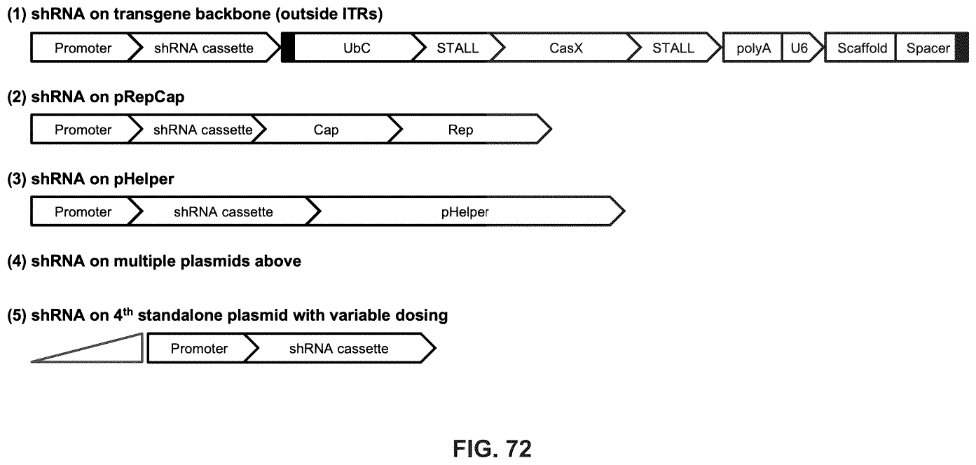

In the embodiments of the system, the expressed shRNA is transcribed and folds within the nucleus of the packaging cells into a short hairpin form that is first processed by the ribonuclease Drosha into pre-siRNA. Next, the pre-siRNA is transported into the cytoplasm, whereupon the pre-siRNA interacts with the endoribonuclease Dicer that processes the double-stranded shRNA into short double-stranded RNA fragments called small interfering RNA (siRNA). A typical siRNA is composed of a passenger strand, and a complementary guide strand, typically 21 nucleotides (nt) in length with 3′ overhangs containing two nucleotides. The guide strand directs binding to a sequence-complementary mRNA, which triggers cleavage by Ago2, resulting in gene silencing (Valenzuela, R., et al. Guide Strand 3′-End Modifications Regulate siRNA Specificity. Chembiochem. 2016 Dec. 14; 17(24): 2340 (2016)). In the context of the present disclosure, the siRNA forms a complex with the RISC protein complex that recruits the siRNA to hybridize with the CRISPR mRNA transcribed from the siAAV transgene, resulting in the targeted cleavage of the CRISPR mRNA such that expression of the CRISPR protein is repressed. In some embodiments, the disclosure provides a polynucleotide, wherein the polynucleotide encodes a shRNA operably linked to a promoter. The encoding sequence and the promoter can be incorporated into different plasmid vectors utilized with the siAAV system, as shown in FIG. 72, to transfect the host packaging cell. In some embodiments, the polynucleotide comprises both the sequence encoding the shRNA and linked promoter and the siAAV transgene, having the general configuration as shown in FIG. 72 and is used to transfect the host packaging cell, along with the pRC and pHelper vectors. In other embodiments, the sequence encoding the shRNA and linked promoter is introduced into the pRC plasmid. In other embodiments, the sequence encoding the shRNA and linked promoter is introduced into the pHelper plasmid. In other embodiments, the sequence encoding the shRNA and linked promoter is introduced into the packaging cell using a separate vector from the AAV transgene vector, while the pRC and pHelper vectors are also transfected into the packaging cell. In other embodiments, the sequence encoding the shRNA is integrated into the packaging cell genome, and the packaging cell is transfected with the AAV transgene and the pRC and pHelper vectors. In still another embodiment, the sequences encoding the shRNA, Rep, Cap, E2, and VA are integrated into the packaging cell genome and the AAV transgene is transfected into the packaging cell. In other embodiments, the polynucleotide comprises a sequence encoding a first shRNA and linked promoter, a second shRNA and linked promoter, and the siAAV transgene. In other embodiments, the polynucleotide comprises a sequence encoding a first shRNA and linked promoter, a second shRNA and linked promoter, a third shRNA and linked promoter, and the siAAV transgene. In other embodiments, the system provides two polynucleotides: a first polynucleotide comprising the gene encodes the shRNA operably linked to a promoter (and, optionally, a second and a third shRNA with operably linked promoters) and a second polynucleotide of the siAAV transgene, having the general configuration as shown in FIG. 72. Designs of such constructs and their resulting properties are provided in the Examples. In some embodiments, upon introduction of the polynucleotide into a host cell, the expressed shRNA of the system is processed into siRNA complementary to a mRNA transcript of the CRISPR protein of the transgene. In a particular embodiment. upon introduction of the polynucleotide into a host cell, the expressed shRNA of the system is processed into siRNA complementary to a mRNA transcript of a CasX protein of any of the embodiments disclosed herein, including the sequences of SEQ ID NOs: 1-3, and the sequence of SEQ ID NOS: 49-321 and 2356-2488, or as set forth in Table 5.

In some embodiments, the shRNA(s) of the system are encoded by DNA sequences selected from the group consisting of SEQ ID NOS: 2640-2687 as set forth in Table 9, or a sequence having at least about 85%, at least about 90%, or at least about 95%, or at least about 95%, or at least about 96%, or at least about 97%, or at least about 98%, or at least about 99% sequence identity thereto. In another embodiment, an encoded shRNA comprises an RNA sequence selected from the group consisting of SEQ ID NOS: 2592-2639 as set forth in Table 9, or a sequence having at least about 85%, at least about 90%, or at least about 95%, or at least about 95%, or at least about 96%, or at least about 97%, or at least about 98%, or at least about 99% sequence identity thereto. In some embodiments, the shRNA cassette is linked to a strong promoter, such as EF-1α, to ensure that shRNA is transcribed in sufficient quantities to repress translation of the CRISPR protein. In some embodiments, the polynucleotide comprises a first and a second, different shRNA wherein the resulting siRNA are both complementary to the mRNA transcript of the CasX.

TABLE 9
shRNA sequences

| | | RNA | DNA |
|---|---|---|---|
| | | SEQ | SEQ |
| | | ID | ID |
| Name | RNA Sequence* | NO: | NO: |
| shRNA1a | GCCUAGGCAACAGAAGGCUAAAGAAGGUAUAUUGCUGUUGAC | 2592 | 2640 |
| | AGUGAGCGACGCUGAUCAUCAAUUACUUACUGUGAAGCCACA | | |
| | GAUGGGUAAGUAAUUGAUGAUCAGCGCUGCCUACUGCCUCGG | | |
| | ACUUCAAGGGGCUACUUUAGGAGCAAUUAUCUUUUUUU | | |
| shRNA1b | GCCUAGGCAACAGAAGGCUCGAGAAGGUAUAUUGCUGUUGAC | 2593 | 2641 |
| | AGUGAGCGUGUACCUGAUCAUCAAUUACUUUAGUGAAGCCAC | | |
| | AGAUGUAAAGUAAUUGAUGAUCAGGUACAUGCCUACUGCCUC | | |
| | GGACUUCAAGGGGGCUACUUUAGGAGCAAUUAUCUUUUUUU | | |
| shRNA1c | GCCUAGGCAACAGAAUGCUAAAGAAGGUAUAUUGCUGUUGAC | 2594 | 2642 |
| | AGUGAGCGACAAGUAAUUGAUGAUCAGGUACACUGUGAAGCC | | |
| | ACAGAUGGGUGUACCUGAAUCAAUUACUUGCUGCCUACUGCC | | |
| | UCGGACUUCAAGGGGCUACUUUAGGAGCAAUUAUCUUUUUUU | | |
| shRNA1d | GCCUAGGCAACAGAAGGCUCGAGAAGGUAUAUUGCUGUUGAC | 2595 | 2643 |
| | AGUGAGCGUGUACCUGAUCAUCAAUUACUUUAGUGAAGCCAC | | |
| | AGAUGUAAAGUAAUUGAUGAUCAGGUACAUGCCUACUGCCUC | | |
| | GGAAUUCAAGGGGGCUACUUUAGGAGCAAUUAUCUUUUUUU | | |
| shRNA2a | GCCUAGGCAACAGAAGGCUAAAGAAGGUAUAUUGCUGUUGAC | 2596 | 2644 |
| | AGUGAGCGACGCAACUGCGCCUUCAUUUACUGUGAAGCCACA | | |
| | GAUGGGUAAAUGAAGGCGCAGUUGCGCUGCCUACUGCCUCGG | | |
| | ACUUCAAGGGGCUACUUUAGGAGCAAUUAUCUUUUUUU | | |
| shRNA2b | GCCUAGGCAACAGAAGGCUCGAGAAGGUAUAUUGCUGUUGAC | 2597 | 2645 |
| | AGUGAGCGCAGUACAACUGCGCCUUCAUUUUAGUGAAGCCAC | | |
| | AGAUGUAAAAUGAAGGCGCAGUUGUACUGUGCCUACUGCCUC | | |
| | GGACUUCAAGGGGGCUACUUUAGGAGCAAUUAUCUUUUUUU | | |
| shRNA2c | GCCUAGGCAACAGAAUGCUAAAGAAGGUAUAUUGCUGUUGAC | 2598 | 2646 |
| | AGUGAGCGACAAAUGAAGGCGCAGUUGUACUGCUGUGAAGCC | | |
| | ACAGAUGGGCAGUACAACCGCCUUCAUUUGCUGCCUACUGCC | | |
| | UCGGACUUCAAGGGGCUACUUUAGGAGCAAUUAUCUUUUUUU | | |
| shRNA2d | GCCUAGGCAACAGAAGGCUCGAGAAGGUAUAUUGCUGUUGAC | 2599 | 2647 |
| | AGUGAGCGCAGUACAACUGCGCCUUCAUUUUAGUGAAGCCAC | | |
| | AGAUGUAAAAUGAAGGCGCAGUUGUACUGUGCCUACUGCCUC | | |
| | GGAAUUCAAGGGGGCUACUUUAGGAGCAAUUAUCUUUUUUU | | |
| shRNA3a | GCCUAGGCAACAGAAGGCUAAAGAAGGUAUAUUGCUGUUGAC | 2600 | 2648 |
| | AGUGAGCGACGCUGAGCAAGCACAUUAAACUGUGAAGCCACA | | |
| | GAUGGGUUUAAUGUGCUUGCUCAGCGCUGCCUACUGCCUCGG | | |
| | ACUUCAAGGGGCUACUUUAGGAGCAAUUAUCUUUUUUU | | |
| shRNA3b | GCCUAGGCAACAGAAGGCUCGAGAAGGUAUAUUGCUGUUGAC | 2601 | 2649 |
| | AGUGAGCGAAGGCCUGAGCAAGCACAUUAAUAGUGAAGCCAC | | |
| | AGAUGUAUUAAUGUGCUUGCUCAGGCCUUUGCCUACUGCCUC | | |
| | GGACUUCAAGGGGGCUACUUUAGGAGCAAUUAUCUUUUUUU | | |
| shRNA3c | GCCUAGGCAACAGAAUGCUAAAGAAGGUAUAUUGCUGUUGAC | 2602 | 2650 |
| | AGUGAGCGACUUAAUGUGCUUGCUCAGGCCUUCUGUGAAGCC | | |
| | ACAGAUGGGAAGGCCUGAAAGCACAUUAAGCUGCCUACUGCC | | |
| | UCGGACUUCAAGGGGCUACUUUAGGAGCAAUUAUCUUUUUUU | | |
| shRNA3d | GCCUAGGCAACAGAAGGCUCGAGAAGGUAUAUUGCUGUUGAC | 2603 | 2651 |
| | AGUGAGCGAAGGCCUGAGCAAGCACAUUAAUAGUGAAGCCAC | | |
| | AGAUGUAUUAAUGUGCUUGCUCAGGCCUUUGCCUACUGCCUC | | |
| | GGAAUUCAAGGGGGCUACUUUAGGAGCAAUUAUCUUUUUUU | | |
| shRNA4a | GCCUAGGCAACAGAAGGCUAAAGAAGGUAUAUUGCUGUUGAC | 2604 | 2652 |
| | AGUGAGCGACGCCUGAUCAUCAAUUACUACUGUGAAGCCACA | | |
| | GAUGGGUAGUAAUUGAUGAUCAGGCGCUGCCUACUGCCUCGG | | |
| | ACUUCAAGGGGCUACUUUAGGAGCAAUUAUCUUUUUUU | | |
| shRNA4b | GCCUAGGCAACAGAAGGCUCGAGAAGGUAUAUUGCUGUUGAC | 2605 | 2653 |
| | AGUGAGCGCUGUACCUGAUCAUCAAUUACUUAGUGAAGCCAC | | |
| | AGAUGUAAGUAAUUGAUGAUCAGGUACAGUGCCUACUGCCUC | | |
| | GGACUUCAAGGGGGCUACUUUAGGAGCAAUUAUCUUUUUUU | | |
| shRNA4c | GCCUAGGCAACAGAAUGCUAAAGAAGGUAUAUUGCUGUUGAC | 2606 | 2654 |
| | AGUGAGCGACAGUAAUUGAUGAUCAGGUACAGCUGUGAAGCC | | |
| | ACAGAUGGGCUGUACCUGCAUCAAUUACUGCUGCCUACUGCC | | |
| | UCGGACUUCAAGGGGCUACUUUAGGAGCAAUUAUCUUUUUUU | | |
| shRNA4d | GCCUAGGCAACAGAAGGCUCGAGAAGGUAUAUUGCUGUUGAC | 2607 | 2655 |
| | AGUGAGCGCUGUACCUGAUCAUCAAUUACUUAGUGAAGCCAC | | |
| | AGAUGUAAGUAAUUGAUGAUCAGGUACAGUGCCUACUGCCUC | | |
| | GGAAUUCAAGGGGGCUACUUUAGGAGCAAUUAUCUUUUUUU | | |
| shRNA5a | GCCUAGGCAACAGAAGGCUAAAGAAGGUAUAUUGCUGUUGAC | 2608 | 2656 |
| | AGUGAGCGACGGUACCUGAUCAUCAAUUACUGUGAAGCCACA | | |
| | GAUGGGUAAUUGAUGAUCAGGUACCGCUGCCUACUGCCUCGG | | |
| | ACUUCAAGGGGCUACUUUAGGAGCAAUUAUCUUUUUUU | | |
| shRNA5b | GCCUAGGCAACAGAAGGCUCGAGAAGGUAUAUUGCUGUUGAC | 2609 | 2657 |
| | AGUGAGCGAACCUGUACCUGAUCAUCAAUUUAGUGAAGCCAC | | |
| | AGAUGUAAAUUGAUGAUCAGGUACAGGUUUGCCUACUGCCUC | | |
| | GGACUUCAAGGGGGCUACUUUAGGAGCAAUUAUCUUUUUUU | | |
| shRNA5c | GCCUAGGCAACAGAAUGCUAAAGAAGGUAUAUUGCUGUUGAC | 2610 | 2658 |
| | AGUGAGCGACAAUUGAUGAUCAGGUACAGGUUCUGUGAAGCC | | |
| | ACAGAUGGGAACCUGUACGAUCAUCAAUUGCUGCCUACUGCC | | |
| | UCGGACUUCAAGGGGCUACUUUAGGAGCAAUUAUCUUUUUUU | | |
| shRNA5d | GCCUAGGCAACAGAAGGCUCGAGAAGGUAUAUUGCUGUUGAC | 2611 | 2659 |
| | AGUGAGCGAACCUGUACCUGAUCAUCAAUUUAGUGAAGCCAC | | |
| | AGAUGUAAAUUGAUGAUCAGGUACAGGUUUGCCUACUGCCUC | | |
| | GGAAUUCAAGGGGGCUACUUUAGGAGCAAUUAUCUUUUUUU | | |
| shRNA6a | GCCUAGGCAACAGAAGGCUAAAGAAGGUAUAUUGCUGUUGAC | 2612 | 2660 |
| | AGUGAGCGACGUUCAUUUGGCAGAAAGAACUGUGAAGCCACA | | |
| | GAUGGGUUCUUUCUGCCAAAUGAACGCUGCCUACUGCCUCGG | | |
| | ACUUCAAGGGGCUACUUUAGGAGCAAUUAUCUUUUUUU | | |
| shRNA6b | GCCUAGGCAACAGAAGGCUCGAGAAGGUAUAUUGCUGUUGAC | 2613 | 2661 |
| | AGUGAGCGGCGCCUUCAUUUGGCAGAAAGAUAGUGAAGCCAC | | |
| | AGAUGUAUCUUUCUGCCAAAUGAAGGCGCUGCCUACUGCCUC | | |
| | GGACUUCAAGGGGGCUACUUUAGGAGCAAUUAUCUUUUUUU | | |
| shRNA6c | GCCUAGGCAACAGAAUGCUAAAGAAGGUAUAUUGCUGUUGAC | 2614 | 2662 |
| | AGUGAGCGACUCUUUCUGCCAAAUGAAGGCGCCUGUGAAGCC | | |
| | ACAGAUGGGGCGCCUUCAUGGCAGAAAGAGCUGCCUACUGCC | | |
| | UCGGACUUCAAGGGGCUACUUUAGGAGCAAUUAUCUUUUUUU | | |
| shRNA6d | GCCUAGGCAACAGAAGGCUCGAGAAGGUAUAUUGCUGUUGAC | 2615 | 2663 |
| | AGUGAGCGGCGCCUUCAUUUGGCAGAAAGAUAGUGAAGCCAC | | |
| | AGAUGUAUCUUUCUGCCAAAUGAAGGCGCUGCCUACUGCCUC | | |
| | GGAAUUCAAGGGGGCUACUUUAGGAGCAAUUAUCUUUUUUU | | |
| shRNA7a | GCCUAGGCAACAGAAGGCUAAAGAAGGUAUAUUGCUGUUGAC | 2616 | 2664 |
| | AGUGAGCGACGCAUCAAUUACUUCAAAGACUGUGAAGCCACA | | |
| | GAUGGGUCUUUGAAGUAAUUGAUGCGCUGCCUACUGCCUCGG | | |
| | ACUUCAAGGGGCUACUUUAGGAGCAAUUAUCUUUUUUU | | |
| shRNA7b | GCCUAGGCAACAGAAGGCUCGAGAAGGUAUAUUGCUGUUGAC | 2617 | 2665 |
| | AGUGAGCGCUGAUCAUCAAUUACUUCAAAGUAGUGAAGCCAC | | |
| | AGAUGUACUUUGAAGUAAUUGAUGAUCAGUGCCUACUGCCUC | | |
| | GGACUUCAAGGGGGCUACUUUAGGAGCAAUUAUCUUUUUUU | | |
| shRNA7c | GCCUAGGCAACAGAAUGCUAAAGAAGGUAUAUUGCUGUUGAC | 2618 | 2666 |
| | AGUGAGCGACCUUUGAAGUAAUUGAUGAUCAGCUGUGAAGCC | | |
| | ACAGAUGGGCUGAUCAUCUUACUUCAAAGGCUGCCUACUGCC | | |
| | UCGGACUUCAAGGGGCUACUUUAGGAGCAAUUAUCUUUUUUU | | |
| shRNA7d | GCCUAGGCAACAGAAGGCUCGAGAAGGUAUAUUGCUGUUGAC | 2619 | 2667 |
| | AGUGAGCGCUGAUCAUCAAUUACUUCAAAGUAGUGAAGCCAC | | |
| | AGAUGUACUUUGAAGUAAUUGAUGAUCAGUGCCUACUGCCUC | | |
| | GGAAUUCAAGGGGGCUACUUUAGGAGCAAUUAUCUUUUUUU | | |
| shRNA8a | GCCUAGGCAACAGAAGGCUAAAGAAGGUAUAUUGCUGUUGAC | 2620 | 2668 |
| | AGUGAGCGACGUACCUGAUCAUCAAUUAACUGUGAAGCCACA | | |
| | GAUGGGUUAAUUGAUGAUCAGGUACGCUGCCUACUGCCUCGG | | |
| | ACUUCAAGGGGCUACUUUAGGAGCAAUUAUCUUUUUUU | | |
| shRNA8b | GCCUAGGCAACAGAAGGCUCGAGAAGGUAUAUUGCUGUUGAC | 2621 | 2669 |
| | AGUGAGCGUCCUGUACCUGAUCAUCAAUUAUAGUGAAGCCAC | | |
| | AGAUGUAUAAUUGAUGAUCAGGUACAGGUUGCCUACUGCCUC | | |
| | GGACUUCAAGGGGGCUACUUUAGGAGCAAUUAUCUUUUUUU | | |
| shRNA8c | GCCUAGGCAACAGAAUGCUAAAGAAGGUAUAUUGCUGUUGAC | 2622 | 2670 |
| | AGUGAGCGACUAAUUGAUGAUCAGGUACAGGUCUGUGAAGCC | | |
| | ACAGAUGGGUCCUGUACCAUCAUCAAUUAGCUGCCUACUGCC | | |
| | UCGGACUUCAAGGGGCUACUUUAGGAGCAAUUAUCUUUUUUU | | |
| shRNA8d | GCCUAGGCAACAGAAGGCUCGAGAAGGUAUAUUGCUGUUGAC | 2623 | 2671 |
| | AGUGAGCGUCCUGUACCUGAUCAUCAAUUAUAGUGAAGCCAC | | |
| | AGAUGUAUAAUUGAUGAUCAGGUACAGGUUGCCUACUGCCUC | | |
| | GGAAUUCAAGGGGGCUACUUUAGGAGCAAUUAUCUUUUUUU | | |
| shRNA9a | GCCUAGGCAACAGAAGGCUAAAGAAGGUAUAUUGCUGUUGAC | 2624 | 2672 |
| | AGUGAGCGACGCUGAUCUUCGAGAAUCUACUGUGAAGCCACA | | |
| | GAUGGGUAGAUUCUCGAAGAUCAGCGCUGCCUACUGCCUCGG | | |
| | ACUUCAAGGGGCUACUUUAGGAGCAAUUAUCUUUUUUU | | |
| shRNA9b | GCCUAGGCAACAGAAGGCUCGAGAAGGUAUAUUGCUGUUGAC | 2625 | 2673 |
| | AGUGAGCGCCAUGCUGAUCUUCGAGAAUCUUAGUGAAGCCAC | | |
| | AGAUGUAAGAUUCUCGAAGAUCAGCAUGGUGCCUACUGCCUC | | |
| | GGACUUCAAGGGGGCUACUUUAGGAGCAAUUAUCUUUUUUU | | |
| shRNA9c | GCCUAGGCAACAGAAUGCUAAAGAAGGUAUAUUGCUGUUGAC | 2626 | 2674 |
| | AGUGAGCGACAGAUUCUCGAAGAUCAGCAUGGCUGUGAAGCC | | |
| | ACAGAUGGGCCAUGCUUCUUCGAGAAUCUGCUGCCUACUGCC | | |
| | UCGGACUUCAAGGGGCUACUUUAGGAGCAAUUAUCUUUUUUU | | |
| shRNA9d | GCCUAGGCAACAGAAGGCUCGAGAAGGUAUAUUGCUGUUGAC | 2627 | 2675 |
| | AGUGAGCGCCAUGCUGAUCUUCGAGAAUCUUAGUGAAGCCAC | | |
| | AGAUGUAAGAUUCUCGAAGAUCAGCAUGGUGCCUACUGCCUC | | |
| | GGAAUUCAAGGGGGCUACUUUAGGAGCAAUUAUCUUUUUUU | | |
| shRNA10a | GCCUAGGCAACAGAAGGCUAAAGAAGGUAUAUUGCUGUUGAC | 2628 | 2676 |
| | AGUGAGCGACGGAAGAAGGGCAAGAAGUACUGUGAAGCCACA | | |
| | GAUGGGUACUUCUUGCCCUUCUUCCGCUGCCUACUGCCUCGG | | |
| | ACUUCAAGGGGCUACUUUAGGAGCAAUUAUCUUUUUUU | | |
| shRNA10b | GCCUAGGCAACAGAAGGCUCGAGAAGGUAUAUUGCUGUUGAC | 2629 | 2677 |
| | AGUGAGCGGACCGGAAGAAGGGCAAGAAGUUAGUGAAGCCAC | | |
| | AGAUGUAACUUCUUGCCCUUCUUCCGGUCUGCCUACUGCCUC | | |
| | GGACUUCAAGGGGGCUACUUUAGGAGCAAUUAUCUUUUUUU | | |
| shRNA10c | GCCUAGGCAACAGAAUGCUAAAGAAGGUAUAUUGCUGUUGAC | 2630 | 2678 |
| | AGUGAGCGACACUUCUUGCCCUUCUUCCGGUCCUGUGAAGCC | | |
| | ACAGAUGGGGACCGGAAGGGGCAAGAAGUGCUGCCUACUGCC | | |
| | UCGGACUUCAAGGGGCUACUUUAGGAGCAAUUAUCUUUUUUU | | |
| shRNA10d | GCCUAGGCAACAGAAGGCUCGAGAAGGUAUAUUGCUGUUGAC | 2631 | 2679 |
| | AGUGAGCGGACCGGAAGAAGGGCAAGAAGUUAGUGAAGCCAC | | |
| | AGAUGUAACUUCUUGCCCUUCUUCCGGUCUGCCUACUGCCUC | | |
| | GGAAUUCAAGGGGGCUACUUUAGGAGCAAUUAUCUUUUUUU | | |
| shRNA11a | GCCUAGGCAACAGAAGGCUAAAGAAGGUAUAUUGCUGUUGAC | 2632 | 2680 |
| | AGUGAGCGACGGAUCAACGAGAAGAAAGACUGUGAAGCCACA | | |
| | GAUGGGUCUUUCUUCUCGUUGAUCCGCUGCCUACUGCCUCGG | | |
| | ACUUCAAGGGGCUACUUUAGGAGCAAUUAUCUUUUUUU | | |
| shRNA11b | GCCUAGGCAACAGAAGGCUCGAGAAGGUAUAUUGCUGUUGAC | 2633 | 2681 |
| | AGUGAGCGAAGCUGAUCAACGAGAAGAAAGUAGUGAAGCCAC | | |
| | AGAUGUACUUUCUUCUCGUUGAUCAGCUUUGCCUACUGCCUC | | |
| | GGACUUCAAGGGGGCUACUUUAGGAGCAAUUAUCUUUUUUU | | |
| shRNA11c | GCCUAGGCAACAGAAUGCUAAAGAAGGUAUAUUGCUGUUGAC | 2634 | 2682 |
| | AGUGAGCGACCUUUCUUCUCGUUGAUCAGCUUCUGUGAAGCC | | |
| | ACAGAUGGGAAGCUGAUCCGAGAAGAAAGGCUGCCUACUGCC | | |
| | UCGGACUUCAAGGGGCUACUUUAGGAGCAAUUAUCUUUUUUU | | |
| shRNA11d | GCCUAGGCAACAGAAGGCUCGAGAAGGUAUAUUGCUGUUGAC | 2635 | 2683 |
| | AGUGAGCGAAGCUGAUCAACGAGAAGAAAGUAGUGAAGCCAC | | |
| | AGAUGUACUUUCUUCUCGUUGAUCAGCUUUGCCUACUGCCUC | | |
| | GGAAUUCAAGGGGGCUACUUUAGGAGCAAUUAUCUUUUUUU | | |
| shRNA12a | GCCUAGGCAACAGAAGGCUAAAGAAGGUAUAUUGCUGUUGAC | 2636 | 2684 |
| | AGUGAGCGACGGAAGAGUUCCAGAAAGAACUGUGAAGCCACA | | |
| | GAUGGGUUCUUUCUGGAACUCUUCCGCUGCCUACUGCCUCGG | | |
| | ACUUCAAGGGGCUACUUUAGGAGCAAUUAUCUUUUUUU | | |
| shRNA12b | GCCUAGGCAACAGAAGGCUCGAGAAGGUAUAUUGCUGUUGAC | 2637 | 2685 |
| | AGUGAGCGACUGGGAAGAGUUCCAGAAAGAUAGUGAAGCCAC | | |
| | AGAUGUAUCUUUCUGGAACUCUUCCCAGUUGCCUACUGCCUC | | |
| | GGACUUCAAGGGGGCUACUUUAGGAGCAAUUAUCUUUUUUU | | |
| shRNA12c | GCCUAGGCAACAGAAUGCUAAAGAAGGUAUAUUGCUGUUGAC | 2638 | 2686 |
| | AGUGAGCGACUCUUUCUGGAACUCUUCCCAGUCUGUGAAGCC | | |
| | ACAGAUGGGACUGGGAAGUUCCAGAAAGAGCUGCCUACUGCC | | |
| | UCGGACUUCAAGGGGCUACUUUAGGAGCAAUUAUCUUUUUUU | | |
| shRNA12d | GCCUAGGCAACAGAAGGCUCGAGAAGGUAUAUUGCUGUUGAC | 2639 | 2687 |
| | AGUGAGCGACUGGGAAGAGUUCCAGAAAGAUAGUGAAGCCAC | | |
| | AGAUGUAUCUUUCUGGAACUCUUCCCAGUUGCCUACUGCCUC | | |
| | GGAAUUCAAGGGGGCUACUUUAGGAGCAAUUAUCUUUUUUU | | |

In some embodiments, the polynucleotides of the shRNA-siAAV system are transfected into a eukaryotic packaging cell, together with a plasmid encoding the AAV capsid protein, wherein the packaging cell is incubated under conditions leading to the expression of the shRNA and the siAAV. In some embodiments, the eukaryotic packaging cell is selected from BHK cells, HEK293 cells, HEK293T cells, NS0 cells, SP2/0 cells, YO myeloma cells, P3X63 mouse myeloma cells, PER cells, PER.C6 cells, hybridoma cells, NIH3T3 cells, COS, HeLa, CHO, or other eukaryotic cells known in the art suitable for the production of recombinant siAAV. The resulting siAAV particles are then recovered by conventional means including, without limitation, affinity chromatography, gradient centrifugation, and ion exchange chromatography.

In another aspect, the present disclosure relates to methods of reducing premature cleavage of an siAAV transgene encoding a Class 2 Type V CRISPR nuclease protein and gRNA in a transfected packaging cell. In some embodiments, the method comprises introducing a sequence encoding a small hairpin RNA (shRNA) into the packaging cell transfected with the siAAV transgene, wherein the shRNA is capable of being expressed and processed into an siRNA sequence, as described above, and wherein the siRNA sequence is complementary to an mRNA of the Class 2 Type V CRISPR nuclease transcribed by the packaging cell. In some embodiments, the method comprises introducing a sequence encoding a small hairpin RNA (shRNA) into the packaging cell transfected with the siAAV transgene, wherein the shRNA is capable of being expressed and processed into an siRNA sequence, as described above, and wherein the siRNA sequence is complementary to an mRNA of the Class 2 Type V CRISPR gRNA transcribed by the packaging cell. In some embodiments, the nucleic acid sequence encoding the shRNA is operably linked to a promoter. In some embodiments, the nucleic acid sequence encoding the shRNA and linked promoter is linked exterior to the AAV transgene in a vector (e.g., is inserted into a bacterial plasmid backbone comprising the AAV transgene but is not within the transgene sequence) that is transfected into the packaging cell, along with the pRC and pHelper vectors. In other embodiments, the nucleic acid sequence encoding the shRNA and linked promoter is introduced into the packaging cell using a separate vector from the AAV transgene vector, while the pRC and pHelper vectors are also transfected into the packaging cell. In other embodiments, the nucleic acid sequence encoding the shRNA is integrated into the packaging cell genome, and the packaging cell is transfected with the AAV transgene and the pRC and pHelper vectors. In still another embodiment, the nucleic acid sequences encoding the shRNA, Rep, Cap, E2, and VA are integrated into the packaging cell genome and the AAV transgene is transfected into the packaging cell. In some embodiments, the packaging cell is selected from the group consisting of BHK, HEK293, HEK293T, NS0, SP2/0, YO myeloma cells, A549, P3X63 mouse myeloma cells, PER, PER.C6, NIH3T3, COS, HeLa, and CHO.

In some embodiments of the disclosure, upon transcription of the shRNA and the Class 2 Type V CRISPR nuclease into mRNA and processing of the shRNA into siRNA by the packaging cell, the siRNA hybridizes with the mRNA of the Class 2 Type V CRISPR nuclease and is degraded by the packaging cell. In some embodiments of the method, expression of the Class 2 Type V CRISPR nuclease protein in the packaging cell is repressed by at least 40%, at least 50%, at least 60%, at least 70%, at least 80%, or at least 90% compared to a transfected packaging cell not comprising the shRNA sequence, when assayed in a timed in vitro assay under comparable conditions. In some embodiments of the method, the Class 2 Type V CRISPR nuclease protein of the siAAV transgene is a CasX, wherein the encoded CasX comprises a sequence selected from the group consisting of SEQ ID NOS: 1-3, and SEQ ID NOS: 49-321 and 2356-2488, or as set forth in Table 5, or a sequence having at least 85%, at least 90%, at least 95%, at least 95%, at least 96%, at least 97%, at least 98%, or at least 99% identity thereto. In other embodiments of the method, the Class 2 Type V CRISPR nuclease protein of the siAAV transgene is a CasX, wherein the encoded CasX comprises a sequence selected from the group consisting of SEQ ID NOS: 1-3, and SEQ ID NOS: 49-321 and 2356-2488, or as set forth in Table 5. In a particular embodiment of the method, a CasX variant protein of the siAAV transgene comprises the sequence of SEQ ID NO: 138, or a sequence having at least about 70%, at least about 80%, at least about 80%, at least about 85%, at least about 90%, or at least about 95%, or at least about 95%, or at least about 96%, or at least about 97%, or at least about 98%, or at least about 99% sequence identity thereto, wherein the mRNA of the transcribed CasX is capable of being bound by the siRNA. In some embodiments, the production of functional CasX protein in a cellular expression system comprising the shRNA cassette is repressed by at least 40%, at least 50%, at least 60%, at least 70%, at least 80%, or at least 90% compared to a system not comprising the shRNA cassette, when assayed in a timed in vitro cellular assay under comparable conditions. Exemplary assay systems are described herein, in the Examples.

In some embodiments of the method, the shRNA used to transfect the packaging cell is encoded by a sequence comprising a sequence selected from the group consisting of SEQ ID NOS: 2640-2687 of Table 9, or a sequence having at least about 85%, at least about 90%, or at least about 95%, or at least about 95%, or at least about 96%, or at least about 97%, or at least about 98%, or at least about 99% sequence identity thereto. In some embodiments, the shRNA comprises a sequence selected from the group consisting of SEQ ID NOS: 2592-2639, or a sequence having at least about 85%, at least about 90%, or at least about 95%, or at least about 95%, or at least about 96%, or at least about 97%, or at least about 98%, or at least about 99% sequence identity thereto, wherein the resulting siRNA (processed from the shRNA by the packaging cell) is capable of binding the mRNA of the transcribed Class 2 Type V, CasX of the siAAV. In other embodiments of the method, the shRNA is encoded by a sequence comprising a sequence selected from the group consisting of SEQ ID NOS: 2640-2687 of Table 9. In some embodiments, the shRNA comprises a sequence selected from the group consisting of SEQ ID NOS: 2592-2639, wherein the resulting siRNA (processed from the shRNA by the packaging cell) is capable of binding the mRNA of the transcribed CasX of the siAAV. In other embodiments of the method, a first and a second, different shRNA sequence is transfected into the packaging cell.

In other embodiments of the method, upon transcription of the shRNA and the Class 2 Type V CRISPR gRNA, and the processing of the shRNA into siRNA by the packaging cell, the siRNA hybridizes with the gRNA and is degraded by the packaging cell. In some embodiments of the method, expression of the Class 2 Type V CRISPR gRNA in the packaging cell is repressed by at least 40%, at least 50%, at least 60%, at least 70%, at least 80%, or at least 90% compared to a transfected packaging cell not comprising the shRNA sequence, when assayed in a timed in vitro assay under comparable conditions.

XI. Controlled Production of siAAV Utilizing RNAi or Anti-Sense RNA

In another aspect, the present disclosure relates to methods of reducing premature cleavage of siAAV transgenes in transfected packaging cells using polynucleotide constructs using interfering RNA (RNAi) or anti-sense RNA (asRNA). In some embodiments, the disclosure provides systems of polynucleotides comprising one or more sequences encoding RNAi or asRNA wherein the sequences are complementary to either the gRNA transcribed by the packaging cell that target the self-inactivating segments utilized in the siAAV transgene or are complementary to the mRNA encoding the Class 2 CRISPR nuclease protein transcribed by the packaging cell. In some embodiments of the method, the RNAi or asRNA sequences are linked to a promoter wherein the sequence encoding the RNAi or asRNA and linked promoter is linked to the 5′ end of the siAAV transgene (i.e., 5′ to the packaging component) transfected into the packaging cell. In other embodiments of the method, the RNAi or asRNA sequences are linked to a promoter wherein the sequence encoding the RNAi or asRNA and linked promoter is transfected into the packaging cell using a separate vector than that of the siAAV. In some embodiments, the packaging cell selected from the group consisting of BHK, HEK293, HEK293T, NS0, SP2/0, YO myeloma cells, A549, P3X63 mouse myeloma cells, PER, PER.C6, NIH3T3, COS, HeLa, and CHO.

In some embodiments of the method, upon transcription of the gRNA and RNAi or asRNA by the packaging cell, the RNAi or asRNA hybridizes with the gRNA, interfering with the ability of the gRNA to complex with the expressed CRISPR nuclease to form an RNP. In other embodiments of the method, upon transcription of the mRNA of the CRISPR nuclease and the RNAi or asRNA by the packaging cell, the RNAi or asRNA hybridizes with the mRNA of the CRISPR nuclease, repressing expression of the CRISPR nuclease protein in the packaging cell. It will be understood that as the amount of CRISPR nuclease protein is reduced in the packaging cell, the ability of the gRNA to complex with the CRISPR nuclease to form an RNP is similarly reduced. As will be appreciated, as it is the RNP that is required to effect the cleavage of the self-inactivating segments, any repression of the formation of the RNP reduces the amount of cleavage of the transgene and increases the ability of the packaging cell to create AAV with an intact transgene. In some embodiments of the method, the formation of the RNP in the packaging cell is repressed by at least 40%, at least 50%, at least 60%, at least 70%, at least 80%, or at least 90% compared to a transfected packaging cell not comprising the RNAi or asRNA sequence, when assayed in a timed in vitro assay under comparable conditions. In other embodiments of the method, the cleavage of the siAAV transgene in the packaging cell is repressed by at least 40%, at least 50%, at least 60%, at least 70%, at least 80%, or at least 90% compared to a transfected packaging cell not comprising the RNAi or asRNA sequence, when assayed in a timed in vitro assay under comparable conditions.

In some embodiments of the method, the encoded Class 2 CRISPR nuclease protein of the transgene is a CasX wherein the encoded sequence is selected from the group consisting of SEQ ID NOS: 1-3, and the sequences of SEQ ID NOS: 49-321 and 2356-2488, or as set forth in Table 5, or a sequence having at least 85%, at least 90%, at least 95%, at least 95%, at least 96%, at least 97%, at least 98%, or at least 99% identity thereto. In some embodiments of the method, the encoded Class 2 CRISPR nuclease protein of the transgene is a CasX wherein the encoded sequence is selected from the group consisting of SEQ ID NOS: 1-3, and the sequences of SEQ ID NOS: 49-321 and 2356-2488, or as set forth in Table 5. In some embodiments of the method, the encoded gRNA has a scaffold comprising a sequence selected from the group of sequences consisting of the sequences of SEQ ID NOS: 2101-2331 and 3992-3995 as set forth in Table 2, or a sequence having at least at least about 50%, at least about 60%, at least about 70%, at least about 80%, at least about 80%, at least about 85%, at least about 90%, or at least about 95%, or at least about 95%, or at least about 96%, or at least about 97%, or at least about 98%, or at least about 99% sequence identity thereto. In some embodiments of the method, the encoded gRNA has a scaffold comprising a sequence selected from the group of sequences consisting of the sequences of SEQ ID NOS: 2101-2331 and 3992-3995 as set forth in Table 2. In some embodiments of the method, the encoded Class 2 CRISPR nuclease protein of the transgene is a CasX of SEQ ID NO: 138 and the encoded gRNA has a scaffold comprising a sequence of SEQ ID NO: 2296.

XII. Controlled Production of siAAV Utilizing Non-Targeting gRNA (“Decoy gRNA”)

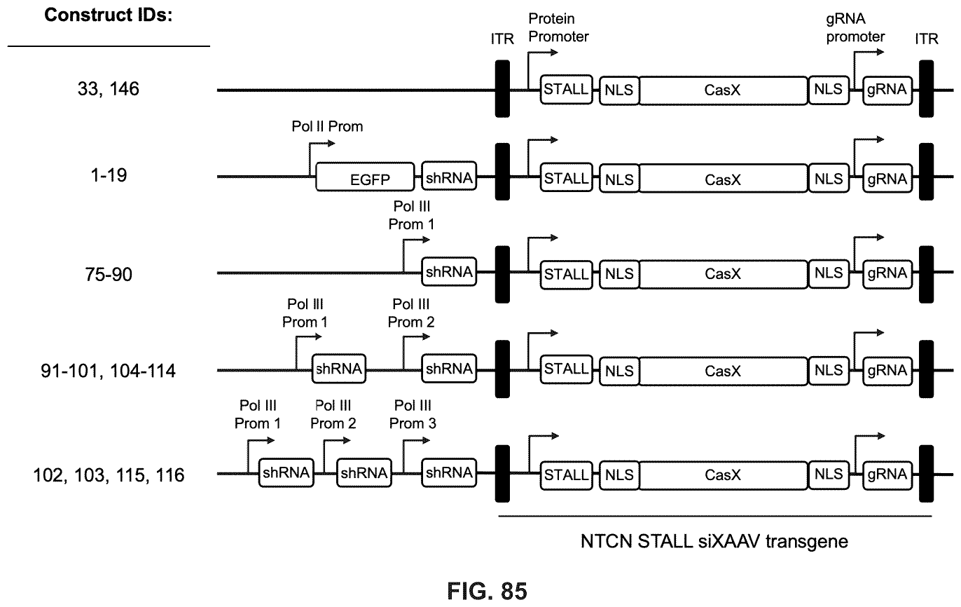

In another aspect, the present disclosure relates to methods of reducing premature cleavage of siAAV transgenes in transfected packaging cells using polynucleotide constructs comprising non-targeting gRNA. In some embodiments, the disclosure provides siAAV systems of polynucleotides comprising, in addition to the siAAV transgene of any of the embodiments described herein that are used to transfect the packaging cell, a sequence encoding a gRNA wherein the gRNA either has a non-targeting targeting sequence (meaning the targeting sequence is not able to hybridize with a target nucleic acid) or the scaffold does not comprise a targeting sequence; i.e., the gRNA is only the scaffold (and in either case would be considered non-targeting). Such gRNA are referred to herein as “decoy gRNA” in that upon expression in the transfected packaging cell, they are able to compete with any expressed targeting gRNA for complexing with expressed CRISPR nuclease protein. When such decoy gRNA form an RNP with the expressed CRISPR nuclease protein, the RNP is unable to cleave the self-inactivating sequences of the siAAV transgene, thereby increasing the number of intact siAAV that can be produced by the host cell. In some embodiments of the method, the non-targeting gRNA sequence is linked to a stronger promoter compared to the promoter linked to the gRNA of the siAAV transgene; embodiments of which are described herein, supra. In other embodiments, the non-targeting gRNA sequence is linked to a promoter that is identical to the promoter linked to the gRNA of the siAAV transgene. In some embodiments, the sequence encoding the non-targeting gRNA and linked promoter is linked to the 5′ end of the siAAV transgene (i.e., 5′ to the packaging element) transfected into the packaging cell. In other embodiments of the method, the non-targeting gRNA sequence and linked promoter is transfected into the packaging cell using a separate vector than that of the siAAV. Representative schematics of such configurations are shown in FIG. 85. In some embodiments, the packaging cell selected from the group consisting of BHK, HEK293, HEK293T, NS0, SP2/0, YO myeloma cells, A549, P3X63 mouse myeloma cells, PER, PER.C6, NIH3T3, COS, HeLa, and CHO.

It will be understood that by use of a stronger promoter with the non-targeting gRNA, compared to the targeting gRNA, the former would be expressed to a greater extent and would complex a larger percentage of the CRISPR nuclease, thereby reducing the amount of premature cleavage of the transgene and increasing the ability of the packaging cell to create siAAV with an intact transgene. In some embodiments, the stronger promoter linked to the non-targeting gRNA is U6, while the promoter linked to the targeting gRNA is selected from the group consisting of H1, 7SK, and mini U6. In some embodiments of the method, the cleavage of the siAAV transgene in the packaging cell is repressed by at least 40%, at least 50%, at least 60%, at least 70%, at least 80%, or at least 90% compared to a transfected packaging cell not comprising the non-targeting gRNA sequence, when assayed in a timed in vitro assay under comparable conditions. In some embodiments of the method, the titer of the siAAV produced by the packaging cell comprising an encoding a decoy RNA is at least about 2-fold, at least about 3-fold, at least about 4-fold, at least about 5-fold, at least about 6-fold, at least about 7-fold, at least about 8-fold, at least about 9-fold, at least about 10-fold higher compared to the titer produced using a comparable siAAV construct not comprising the decoy gRNA.

In some embodiments of the method, the encoded Class 2 Type V CRISPR nuclease protein of the transgene is a CasX wherein the encoded sequence is selected from the group consisting of SEQ ID NOS: 1-3 and the sequences of SEQ ID NOS: 49-321 and 2356-2488, or as set forth in Table 5, or a sequence having at least 85%, at least 90%, at least 95%, at least 95%, at least 96%, at least 97%, at least 98%, or at least 99% identity thereto. In some embodiments of the method, the encoded Class 2 Type V CRISPR nuclease protein of the transgene is a CasX wherein the encoded sequence is selected from the group consisting of SEQ ID NOS: 1-3 and the sequences of SEQ ID NOS: 49-321 and 2356-2488, or as set forth in Table 5. In some embodiments of the method, the encoded gRNA of the transgene and the non-targeting decoy gRNA each has a scaffold comprising a sequence selected from the group of sequences consisting of the sequences of SEQ ID NOS: 2101-2331 and 3992-3995 as set forth in Table 2, or a sequence having at least at least about 50%, at least about 60%, at least about 70%, at least about 80%, at least about 80%, at least about 85%, at least about 90%, or at least about 95%, or at least about 95%, or at least about 96%, or at least about 97%, or at least about 98%, or at least about 99% sequence identity thereto. In some embodiments of the method, the encoded gRNA of the transgene and the non-targeting decoy gRNA each has a scaffold comprising a sequence selected from the group of sequences consisting of the sequences of SEQ ID NOS: 2101-2331 and 3992-3995 as set forth in Table 2. In some embodiments of the method, the encoded decoy gRNAs has a stronger binding affinity to the CRISPR nuclease than the targeting gRNA. In some embodiments, the decoy gRNA comprises a scaffold of SEQ ID NO: 2291 or 2296, while the targeting gRNA comprises a scaffold of SEQ ID NO: 2238.

### XIII. Kits and Articles of Manufacture

In other embodiments, provided herein are kits comprising an SIRV or siAAV vector of any of the embodiments of the disclosure, and a suitable container (for example a tube, vial or plate).

In some embodiments, the kit further comprises a buffer, a nuclease inhibitor, a protease inhibitor, a liposome, a therapeutic agent, a label, a label visualization reagent, or any combination of the foregoing. In some embodiments, the kit further comprises a pharmaceutically acceptable carrier, diluent or excipient.

In some embodiments, the kit comprises appropriate control compositions for gene modifying applications, and instructions for use.

The present description sets forth numerous exemplary configurations, methods, parameters, and the like. It should be recognized, however, that such description is not intended as a limitation on the scope of the present disclosure, but is instead provided as a description of exemplary embodiments.

### Enumerated Embodiments

The disclosure can be understood with respect to the following illustrated, enumerated embodiments:

### Set I

Embodiment I-1. A self-inactivating recombinant vector (SIRV) comprising a polynucleotide comprising one or more components selected from:
a) one or more packaging components;
b) a sequence encoding a Class 2 CRISPR protein having a single RNA-guided RuvC domain;
c) a first promoter operably linked to the sequence encoding the Class 2 CRISPR protein;
d) a sequence encoding a first guide RNA (gRNA) comprising a scaffold sequence and a linked targeting sequence that is complementary to and capable of hybridizing with: 1) a target nucleic acid of a cell to be modified; and 2) one or more self-inactivating segments incorporated in the polynucleotide;
e) a second promoter sequence operably linked to the sequence encoding the first gRNA; and
f) one or more self-inactivating segments of the polynucleotide comprising a protospacer adjacent motif (PAM) sequence and a polynucleotide sequence capable of being bound and cleaved by a ribonuclear protein complex (RNP) comprising the Class 2 CRISPR protein and the first gRNA.

Embodiment I-2. The SIRV of embodiment I-1, wherein the SIRV comprises components (a)-(f).

Embodiment I-3. The SIRV of embodiment I-1 or I-2, wherein the one or more self-inactivating segments of the polynucleotide are located:
a) 5′ or 3′ adjacent to or within the sequence encoding the Class 2 CRISPR protein;
b) 5′ or 3′ adjacent to or within a Kozak sequence located between the first promoter and the sequence encoding the Class 2 CRISPR protein;
c) 5′ or 3′ adjacent to or within to the first promoter sequence;
d) 5′ or 3′ adjacent to or within the second promoter sequence;
e) downstream of the transcriptional start site for the sequence encoding the Class 2 CRISPR protein;
f) within one or more inserted introns in the polynucleotide encoding the Class 2 CRISPR protein;
g) at the 3′ end of the polynucleotide encoding the Class 2 CRISPR protein, between a stop codon and poly(A) termination site for the Class2 CRISPR protein; or
h) any combination of (a)-(g).

Embodiment I-4. The SIRV of embodiments I-1 to I-3, wherein the self-inactivating segment comprises a sequence corresponding to any 15-21 nucleotide portion of the target nucleic acid sequence that is 3′ adjacent to a PAM sequence recognized by an RNP of the Class 2 CRISPR protein and the first gRNA.

Embodiment I-5. The SIRV of any one of embodiments I-1 to I-4, wherein the PAM sequence of the one or more self-inactivating segments:
a) is identical to the PAM sequence of the target nucleic acid of the cell to be modified; and
b) promotes less efficient cleavage or rate of cleavage of the self-inactivating segment by the RNP compared to the PAM sequence 5′ and adjacent to the target nucleic acid of the cell to be modified.

Embodiment I-6. The SIRV of embodiment I-5, wherein the PAM sequence of the target nucleic acid is NTN.

Embodiment I-7. The SIRV of any one of embodiments I-1 to I-6, wherein the PAM sequence of the one or more self-inactivating segments:
a) is different by at least one nucleotide from the PAM sequence of the target nucleic acid of the cell to be modified; and
b) promotes less efficient cleavage or rate of cleavage of the self-inactivating segment by the RNP compared to the target nucleic acid of the cell to be modified.

Embodiment I-8. The SIRV of embodiment I-7, wherein:
a) if the PAM sequence of the target nucleic acid of the cell to be modified is TTC and the PAM preference of the Class 2 CRISPR protein is TTC, then the PAM sequence of the one or more self-inactivating segments is selected from the group consisting of ATC, CTC, TTT, GTT, and GTC;
b) if the PAM sequence of the target nucleic acid of the cell to be modified is ATC and the PAM preference of the Class 2 CRISPR protein is TTC, then the PAM sequence of the one or more self-inactivating segments is selected from the group consisting of CTC, TTT, GTT, and GTC; or
c) if the PAM sequence of the target nucleic acid of the cell to be modified is CTC and the PAM preference of the Class 2 CRISPR protein is TTC, then the PAM sequence of the one or more self-inactivating segments is TTT, GTT, ATC, or GTC.

Embodiment I-9. The SIRV of embodiment I-7, wherein:
a) if the PAM sequence of the target nucleic acid of the cell to be modified is ATC and the PAM preference of the Class 2 CRISPR protein is ATC, then the PAM sequence of the one or more self-inactivating segments is selected from the group consisting of TTC, CTC, TTT, GTT, and GTC;
b) if the PAM sequence of the target nucleic acid of the cell to be modified is CTC and the PAM preference of the Class 2 CRISPR protein is ATC, then the PAM sequence of the one or more self-inactivating segments is selected from the group consisting of TTC, TTT, GTT, and GTC; or
c) if the PAM sequence of the target nucleic acid of the cell to be modified is GTC and the PAM preference of the Class 2 CRISPR protein is ATC, then the PAM sequence of the one or more self-inactivating segments is TTC, TTT, CTC, or GTT.

Embodiment I-10. The SIRV of embodiment I-7, wherein:
a) if the PAM sequence of the target nucleic acid of the cell to be modified is GTC and the PAM preference of the Class 2 CRISPR protein is GTC, then the PAM sequence of the one or more self-inactivating segments is selected from the group consisting of ATC, CTC, TTT, GTT, and TTC.
b) if the PAM sequence of the target nucleic acid of the cell to be modified is GTC and the PAM preference of the Class 2 CRISPR protein is ATC, then the PAM sequence of the one or more self-inactivating segments is selected from the group consisting of TTC, TTT, GTT, and CTC; or
c) if the PAM sequence of the target nucleic acid of the cell to be modified is CTC and the PAM preference of the Class 2 CRISPR protein is GTC, then the PAM sequence of the one or more self-inactivating segments is TTC, TTT, ATC, or GTT.

Embodiment I-11. The SIRV of embodiment I-7, wherein:
a) if the PAM sequence of the target nucleic acid of the cell to be modified is CTC and the PAM preference of the Class 2 CRISPR protein is CTC, then the PAM sequence of the one or more self-inactivating segments is selected from the group consisting of ATC, GTC, TTT, GTT, and TTC.
b) if the PAM sequence of the target nucleic acid of the cell to be modified is ATC and the PAM preference of the Class 2 CRISPR protein is CTC, then the PAM sequence of the one or more self-inactivating segments is selected from the group consisting of GTC, TTT, GTT, and TTC; or
c) if the PAM sequence of the target nucleic acid of the cell to be modified is GTC and the PAM preference of the Class 2 CRISPR protein is CTC, then the PAM sequence of the one or more self-inactivating segments is TTC, TTT, ATC, or GTT.

Embodiment I-12. The SIRV of any one of embodiments I-1 to I-11, wherein the one or more self-inactivating segments each have between about 1 to about 5 bases that are not individually complementary to corresponding positions in the targeting sequence of the first gRNA.

Embodiment I-13. The SIRV of embodiment I-12, wherein the one or more self-inactivating segments each have between 1 to 3 bases that are not complementary to corresponding positions in the targeting sequence of the first gRNA.

Embodiment I-14. The SIRV of embodiment I-12 or I-13, wherein the base differences of the one or more self-inactivating segments correspond to positions that are 3′ to the fourth nucleotide of the targeting sequence of the first gRNA when the two sequences are aligned.

Embodiment I-15. The SIRV of any one of embodiments I-1 to I-14, wherein the percent cleavage by the RNP of the self-inactivating segments of the polynucleotide in a cell transfected or transduced with the SIRV is at least about 10%, at least about 20%, at least about 30%, at least about 40%, at least about 50%, at least about 60%, at least about 70%, at least about 80%, or at least about 90% less than the cleavage of the target nucleic acid in the cell in a timed in vitro cell-based assay, when assayed under comparable conditions.

Embodiment I-16. The SIRV of any one of embodiments I-1 to I-15, wherein the time to achieve 90% cleavage by the RNP of the self-inactivating segments of the polynucleotide in a cell transfected or transduced with the SIRV is delayed, relative to the time to achieve 90% editing of the target nucleic acid in the cell, by at least about 1 day, at least about 2 days, at least about 3 days, at least about 4 days, at least about 5 days, at least about 6 days, at least about 7 days, at least about 8 days, or at least about 9 days, when assayed in an in vitro assay under comparable conditions.

Embodiment I-17. The SIRV of any one of embodiments I-1 to I-16, wherein cleavage by the RNP of the self-inactivating segments of the polynucleotide in a cell transfected or transduced with the SIRV has a k_{cleave} rate that is at least about 2-fold, at least about 4-fold, at least about 5-fold, at least about 6-fold, at least about 7-fold, at least about 8-fold, at least about 9-fold, or at least about 10-fold less than the k_{cleave} rate of the target nucleic acid in an in vitro cell-based assay, when assayed under comparable conditions.

Embodiment I-18. The SIRV of any one of embodiments I-1 to I-17, wherein cleavage by the RNP of the self-inactivating segment of the polynucleotide in a cell transduced or transfected with the SIRV results in reduced or eliminated expression of the Class 2 CRISPR protein or the gRNA encoded by the polynucleotide.

Embodiment I-19. The SIRV of any one of embodiments I-1 to I-18, wherein the Class 2 CRISPR protein further comprises one or more nuclear localization signals (NLS).

Embodiment I-20. The SIRV of embodiment I-19, wherein the one or more NLS are expressed at or near the C-terminus of the Class 2 CRISPR protein.

Embodiment I-21. The SIRV of embodiment I-19, wherein the one or more NLS are expressed at or near the N-terminus of the Class 2 CRISPR protein.

Embodiment I-22. The SIRV of embodiment I-19, comprising one or more NLS located at or near the N-terminus and at or near the C-terminus of the Class 2 CRISPR protein.

Embodiment I-23. The SIRV of any one of embodiments I-19 to I-22, wherein the one or more NLS are selected from the group of sequences consisting of PKKKRKV (SEQ ID NO: 344), KRPAATKKAGQAKKKK (SEQ ID NO: 345), PAAKRVKLD (SEQ ID NO: 346), RQRRNELKRSP (SEQ ID NO: 347), NQSSNFGPMKGGNFGGRSSGPYGGGGQYFAKPRNQGGY (SEQ ID NO: 348), RMRIZFKNKGKDTAELRRRRVEVSVELRKAKKDEQILKRRNV (SEQ ID NO: 349), VSRKRPRP (SEQ ID NO: 350), PPKKARED (SEQ ID NO: 351), PQPKKKPL (SEQ ID NO:352), SALIKKKKKMAP (SEQ ID NO: 353), DRLRR (SEQ ID NO: 354), PKQKKRK (SEQ ID NO: 355), RKLKKKIKKL (SEQ ID NO: 356), REKKKFLKRR (SEQ ID NO: 357), KRKGDEVDGVDEVAKKKSKK (SEQ ID NO: 358), RKCLQAGMNLEARKTKK (SEQ ID NO: 359), PRPRKIPR (SEQ ID NO: 360), PPRKKRTVV (SEQ ID NO: 361), NLSKKKKRKREK (SEQ ID NO: 362), RRPSRPFRKP (SEQ ID NO: 363), KRPRSPSS (SEQ ID NO: 364), KRGINDRNFWRGENERKTR (SEQ ID NO: 365), PRPPKMARYDN (SEQ ID NO: 366), KRSFSKAF (SEQ ID NO: 367), KLKIKRPVK (SEQ ID NO: 368), PKTRRRPRRSQRKRPPT (SEQ ID NO: 370), SRRRKANPTKLSENAKKLAKEVEN (SEQ ID NO: 371), KTRRRPRRSQRKRPPT (SEQ ID NO: 372), RRKKRRPRRKKRR (SEQ ID NO: 373), PKKKSRKPKKKSRK (SEQ ID NO: 374), HKKKHPDASVNFSEFSK (SEQ ID NO: 375), QRPGPYDRPQRPGPYDRP (SEQ ID NO: 376), LSPSLSPLLSPSLSPL (SEQ ID NO: 377), RGKGGKGLGKGGAKRHRK (SEQ ID NO: 378), PKRGRGRPKRGRGR (SEQ ID NO: 379), MSRRRKANPTKLSENAKKLAKEVEN (SEQ ID NO: 598), PKKKRKVPPPPAAKRVKLD (SEQ ID NO: 380), PKKKRKVPPPPKKKRKV (SEQ ID NO: 381), PAKRARRGYKC (SEQ ID NO: 382); KLGPRKATGRW (SEQ ID NO: 383), PRRKREE (SEQ ID NO: 384), PLRKRPRR (SEQ ID NO: 386), PLRKRPRRGSPLRKRPRR (SEQ ID NO: 387), PAAKRVKLDGGKRTADGSEFESPKKKRKV (SEQ ID NO: 388), PAAKRVKLDGGKRTADGSEFESPKKKRKVGIHGVPAA (SEQ ID NO: 389), PAAKRVKLDGGKRTADGSEFESPKKKRKVAEAAAKEAAAKEAAAKA (SEQ ID NO: 390), PAAKRVKLDGGKRTADGSEFESPKKKRKVPG (SEQ ID NO: 391), KRKGSPERGERKRHW (SEQ ID NO: 392), KRTADSQHSTPPKTKRKVEFEPKKKRKV (SEQ ID NO: 393), PKKKRKVGGSKRTADSQHSTPPKTKRKVEFEPKKKRKV (SEQ ID NO: 394), MAPKKKRKVSR (SEQ ID NO: 771), and MAPKKKRKVGGSPKKKRKVGGSPKKKRKVGGSPKKKRKVSR (SEQ ID NO: 772) wherein the one or more NLS are linked to the CRISPR protein or to adjacent NLS with a linker peptide wherein the linker peptide is selected from the group consisting of RS, (G)n (SEQ ID NO: 395), (GS)n (SEQ ID NO: 396), (GSGGS)n (SEQ ID NO: 397), (GGSGGS)n (SEQ ID NO: 398), (GGGS)n (SEQ ID NO: 399), GGSG (SEQ ID NO: 400), GGSGG (SEQ ID NO: 401), GSGSG (SEQ ID NO: 402), GSGGG (SEQ ID NO: 403), GGGSG (SEQ ID NO: 404), GSSSG (SEQ ID NO:405), GPGP (SEQ ID NO: 406), GGP, PPP, PPAPPA (SEQ ID NO: 407), PPPG (SEQ ID NO: 408), PPPGPPP (SEQ ID NO: 409), PPP(GGGS)n (SEQ ID NO: 410), (GGGS)nPPP (SEQ ID NO: 411), AEAAAKEAAAKEAAAKA (SEQ ID NO: 412), and TPPKTKRKVEFE (SEQ ID NO: 413), where n is 1 to 5.

Embodiment I-24. The SIRV of any one of embodiments I-19 to I-22, wherein the one or more encoded NLS are selected from the group consisting of SEQ ID NOS: 538-613 set for in Table 22 and Table 23, or a sequence having at least 85%, at least 90%, at least 95%, at least 95%, at least 96%, at least 97%, at least 98% identity thereto.

Embodiment I-25. The SIRV of any one of embodiments I-19 to I-22, wherein the one or more encoded NLS are selected from the group consisting of SEQ ID NOS: 538-597, 599-610, 613, 771-772, 844-846 and 2498-2591 set forth in Table 7, Table 22, and Table 23.

Embodiment I-26. The SIRV of any one of embodiments I-1 to I-25, wherein the Class 2 CRISPR protein is a CasX protein selected from the group of sequences consisting of SEQ ID NOs: 1-3, 49-321 and 2356-2488, or a sequence having at least about 70%, at least about 80%, at least about 80%, at least about 85%, at least about 90%, or at least about 95%, or at least about 95%, or at least about 96%, or at least about 97%, or at least about 98%, or at least about 99% sequence identity thereto.

Embodiment I-27. The SIRV of any one of embodiments I-1 to I-25, wherein the Class 2 CRISPR protein is a CasX protein having a sequence selected from the group of sequences consisting of SEQ ID NOs: 1-3 and 49-321 and 2356-2488.

Embodiment I-28. The SIRV of any one of embodiments I-1 to I-25, wherein the Class 2 CRISPR protein is a CasX protein comprising the sequence of SEQ ID NO: 138, or a sequence having at least about 70%, at least about 80%, at least about 80%, at least about 85%, at least about 90%, or at least about 95%, or at least about 95%, or at least about 96%, or at least about 97%, or at least about 98%, or at least about 99% sequence identity thereto.

Embodiment I-29. The SIRV of any one of embodiments I-1 to I-28, wherein the first gRNA has a scaffold comprising a sequence selected from the group of sequences consisting of SEQ ID NOS: 2101-2331 and 3992-3995, or a sequence having at least about 70%, at least about 80%, at least about 80%, at least about 85%, at least about 90%, or at least about 95%, or at least about 95%, or at least about 96%, or at least about 97%, or at least about 98%, or at least about 99% sequence identity thereto.

Embodiment I-30. The SIRV of any one of embodiments I-1 to I-28, wherein the first gRNA has a scaffold comprising a sequence selected from the group of sequences consisting of SEQ ID NOS: 2101-2331 and 3992-3995.

Embodiment I-31. The SIRV of any one of embodiments I-1 to I-28, wherein the first gRNA has a scaffold comprising the sequence of SEQ ID NO: 2296, or a sequence having at least about 70%, at least about 80%, at least about 80%, at least about 85%, at least about 90%, or at least about 95%, or at least about 95%, or at least about 96%, or at least about 97%, or at least about 98%, or at least about 99% sequence identity thereto.

Embodiment I-32. The SIRV of any one of embodiments I-1 to I-31, wherein the first gRNA comprises a targeting sequence having 15 nucleotides, 16 nucleotides, 17, nucleotides, 18 nucleotides, 19 nucleotides, or 20 nucleotides.

Embodiment I-33. The SIRV of any one of embodiments I-26 to I-31, wherein the CasX protein is capable of forming a ribonuclear protein complex (RNP) with the first gRNA upon expression in the cell.

Embodiment I-34. The SIRV of embodiment I-33, wherein the RNP is capable of cleaving the target nucleic acid and the self-inactivating segment.

Embodiment I-35. The SIRV of any one of embodiments I-1 to I-34, wherein the polynucleotide further comprises at least one accessory element sequence.

Embodiment I-36. The SIRV of embodiment I-35, wherein the at least one accessory element is selected from the group consisting of a poly(A) signal, a gene enhancer element, an intron, a posttranscriptional regulatory element (PTRE), a deaminase, a DNA glycosylase inhibitor, a promoter, a stimulator of CRISPR-mediated homology-directed repair, an activator or repressor of transcription, and a self-cleaving sequence.

Embodiment I-37. The SIRV of embodiment I-36, wherein the PTRE is selected from the group consisting of cytomegalovirus immediate/early intronA, hepatitis B virus PRE (HPRE), Woodchuck Hepatitis virus PRE (WPRE), and 5′ untranslated segment (UTR) of human heat shock protein 70 mRNA (Hsp70).

Embodiment I-38. The SIRV of embodiment I-36, wherein the PTRE comprises a sequence selected from the group consisting of SEQ ID NOS: 524-526.

Embodiment I-39. The SIRV of any one of embodiments I-35 to I-38, wherein the at least one accessory element enhances the expression, binding, activity, or performance of the CRISPR protein in the cell transduced or transfected with the SIRV as compared to the CRISPR protein in the absence of said accessory element.

Embodiment I-40. The SIRV of embodiment I-39, wherein the enhancement results in an increase in editing of a target nucleic acid in a cell-based timed in vitro assay of at least about 10%, at least about 20%, at least about 30%, at least about 40%, at least about 50%, at least about 60%, at least about 70%, at least about 80%, at least about 90%, at least about 100%, at least about 150%, at least about 200%, or at least about 300%.

Embodiment I-41. The SIRV of any one of embodiments I-1 to I-40, wherein the packaging element is selected from the group consisting of AAV 5′ and 3′ inverted terminal repeats (ITR), adenovirus packaging protein, lentiviral psi packaging element, and gammaretroviral psi packaging element.

Embodiment I-42. The SIRV of embodiment I-41, wherein the AAV 5′ and 3′ ITRs are derived from serotype AAV1, AAV2, AAV3, AAV4, AAV5, AAV6, AAV7, AAV8, AAV9, AAV10, AAV11, AAV12, AAV 44.9, AAV 9.45, AAV 9.61, AAV-Rh74, AAVRh10, or chimeric combinations thereof.

Embodiment I-43. The SIRV of embodiment I-42, wherein the ITRs are derived from serotype AAV2.

Embodiment I-44. An SIRV comprising a polynucleotide comprising one or more components selected from:
a) one or more packaging components;
b) a sequence encoding a Class 2 CRISPR protein;
c) a first promoter operably linked to the sequence encoding the Class 2 CRISPR protein;
d) a sequence encoding a first guide RNA (gRNA) comprising a scaffold sequence and a linked targeting sequence that is complementary to a target nucleic acid of a cell to be modified;
e) a second promoter sequence operably linked to the sequence encoding the first gRNA;
f) a sequence encoding a second gRNA comprising a targeting sequence complementary to one or more self-inactivating segments of the SIRV, wherein the second gRNA comprises a scaffold sequence identical to the scaffold sequence of the first gRNA and the targeting sequence has a lower binding affinity to one or more self-inactivating segments compared to the binding affinity of the targeting sequence of the first gRNA to the target nucleic acid;
g) a sequence encoding a second gRNA comprising a targeting sequence complementary to the one or more self-inactivating segments, the second gRNA comprising a scaffold sequence different from the scaffold sequence of the first gRNA, wherein the second gRNA promotes less efficient editing and/or cleavage by an RNP comprising the Class 2 CRISPR protein and the second gRNA compared to an RNP comprising the Class 2 CRISPR protein and the first gRNA;
h) a sequence encoding a second gRNA comprising a targeting sequence complementary to both a target nucleic acid of a cell to be modified and to one or more self-inactivating segments of the SIRV, wherein the second gRNA comprises a scaffold sequence identical to the scaffold sequence of the first gRNA, wherein:
   i) the PAM sequence of the one or more self-inactivating segments is different by at least one nucleotide from the PAM sequence of the target nucleic acid of the cell to be modified and promotes less efficient cleavage or rate of cleavage of the self-inactivating segment by the RNP compared to the target nucleic acid of the cell to be modified; and
   ii) the targeting sequence of the second gRNA is complementary to different or overlapping regions of the target nucleic acid sequence compared to the targeting sequence of the first gRNA;
i) a third promoter sequence operably linked to the sequence encoding the second gRNA; and
j) one or more self-inactivating segments comprising a protospacer adjacent motif (PAM) sequence and a polynucleotide sequence capable of being bound and cleaved by a ribonuclear protein complex (RNP) of the Class 2 CRISPR protein and the second gRNA.

Embodiment I-45. The SIRV of embodiment I-44, comprising components (a)-(f), (i) and (j).

Embodiment I-46. The SIRV of embodiment I-44, comprising components (a)-(e), (g), (i) and (j).

Embodiment I-47. The SIRV of embodiment I-44, comprising components (a)-(e), and (h)-(j).

Embodiment I-48. The SIRV of any one of embodiments I-44 to I-46, wherein the one or more self-inactivating segments of the polynucleotide are located:
a) 5′ or 3′ adjacent to or within the sequence encoding the Class 2 CRISPR protein;
b) 5′ or 3′ adjacent to or within a Kozak sequence located between the first promoter and the sequence encoding the Class 2 CRISPR protein;
c) 5′ or 3′ adjacent to or within to the first promoter sequence;
d) 5′ or 3′ adjacent to or within the second promoter sequence;
e) 5′ or 3′ adjacent to or within the third promoter sequence;
f) downstream of the transcriptional start site for the sequence encoding the Class 2 CRISPR protein;
g) within one or more inserted introns in the polynucleotide encoding the Class 2 CRISPR protein;
h) at the 3′ end of the polynucleotide encoding the Class 2 CRISPR protein, between a stop codon and poly(A) termination site of the sequence encoding the Class 2 CRISPR; or
i) any combination of (a)-(h).

Embodiment I-49. The SIRV of embodiment I-47, wherein the self-inactivating segment comprises a 15-21 nucleotide sequence complementary to the targeting sequence of the second gRNA that is 3′ adjacent to a PAM sequence recognized by an RNP of the Class 2 CRISPR protein and the second gRNA.

Embodiment I-50. The SIRV of any one of embodiments I-44 to I-49, wherein cleavage of the self-inactivating segments in a cell transduced or transfected with the SIRV by the RNP of the Class 2 CRISPR protein and the second gRNA results in reduced or eliminated expression of the Class 2 CRISPR protein or the gRNA encoded by the polynucleotide.

Embodiment I-51. The SIRV of any one of embodiments I-44 to I-50, wherein the PAM sequence of the one or more self-inactivating segments:
a) is identical to the PAM sequence of the target nucleic acid of the cell to be modified; and
b) promotes less efficient cleavage or rate of cleavage of the self-inactivating segment by the RNP of the Class 2 CRISPR protein and the second gRNA compared to the PAM sequence 5′ and adjacent to the target nucleic acid of the cell to be modified.

Embodiment I-52. The SIRV of embodiment I-51, wherein the PAM sequence of the target nucleic acid is NTN.

Embodiment I-53. The SIRV of any one of embodiments I-44 to I-52, wherein the PAM sequence of the one or more self-inactivating segments:
a) is different from the PAM sequence of the target nucleic acid of the cell to be modified; and
b) promotes less efficient cleavage or rate of cleavage of the self-inactivating segment by the RNP of the Class 2 CRISPR protein and the second gRNA compared to the PAM of the target nucleic acid of the cell to be modified.

Embodiment I-54. The SIRV of embodiment I-53, wherein:
a) if the PAM sequence of the target nucleic acid of the cell to be modified is TTC and the PAM preference of the Class 2 CRISPR protein is TTC, then the PAM sequence of the one or more self-inactivating segments is selected from the group consisting of ATC, CTC, TTT, GTT, and GTC;
b) if the PAM sequence of the target nucleic acid of the cell to be modified is ATC and the PAM preference of the Class 2 CRISPR protein is TTC, then the PAM sequence of the one or more self-inactivating segments is selected from the group consisting of CTC, TTT, GTT, and GTC; or
c) if the PAM sequence of the target nucleic acid of the cell to be modified is CTC and the PAM preference of the Class 2 CRISPR protein is TTC, then the PAM sequence of the one or more self-inactivating segments is GTC, TTT, ATC, or GTT.

Embodiment I-55. The SIRV of embodiment I-53, wherein:
a) if the PAM sequence of the target nucleic acid of the cell to be modified is ATC and the PAM preference of the Class 2 CRISPR protein is ATC, then the PAM sequence of the one or more self-inactivating segments is selected from the group consisting of TTC, CTC, TTT, GTT, and GTC;
b) if the PAM sequence of the target nucleic acid of the cell to be modified is CTC and the PAM preference of the Class 2 CRISPR protein is ATC, then the PAM sequence of the one or more self-inactivating segments is selected from the group consisting of TTC, TTT, GTT, and GTC; or
c) if the PAM sequence of the target nucleic acid of the cell to be modified is GTC and the PAM preference of the Class 2 CRISPR protein is ATC, then the PAM sequence of the one or more self-inactivating segments is TTC, TTT, CTC, or GTT.

Embodiment I-56. The SIRV of embodiment I-53, wherein:
a) if the PAM sequence of the target nucleic acid of the cell to be modified is GTC and the PAM preference of the Class 2 CRISPR protein is GTC, then the PAM sequence of the one or more self-inactivating segments is selected from the group consisting of ATC, CTC, TTT, GTT, and TTC.
b) if the PAM sequence of the target nucleic acid of the cell to be modified is GTC and the PAM preference of the Class 2 CRISPR protein is ATC, then the PAM sequence of the one or more self-inactivating segments is selected from the group consisting of TTC, TTT, GTT, and CTC; or
c) if the PAM sequence of the target nucleic acid of the cell to be modified is CTC and the PAM preference of the Class 2 CRISPR protein is GTC, then the PAM sequence of the one or more self-inactivating segments is TTC, TTT, ATC, or GTT.

Embodiment I-57. The SIRV of embodiment I-53, wherein:
a) if the PAM sequence of the target nucleic acid of the cell to be modified is CTC and the PAM preference of the Class 2 CRISPR protein is CTC, then the PAM sequence of the one or more self-inactivating segments is selected from the group consisting of ATC, GTC, TTT, GTT, and TTC.
b) if the PAM sequence of the target nucleic acid of the cell to be modified is ATC and the PAM preference of the Class 2 CRISPR protein is CTC, then the PAM sequence of the one or more self-inactivating segments is selected from the group consisting of GTC, TTT, GTT, and TTC; or
c) if the PAM sequence of the target nucleic acid of the cell to be modified is GTC and the PAM preference of the Class 2 CRISPR protein is CTC, then the PAM sequence of the one or more self-inactivating segments is TTC, TTT, ATC, or GTT.

Embodiment I-58. The SIRV of any one of embodiments I-44 to I-57, wherein the one or more self-inactivating segment sequences each have between 1 to 5 bases that are not complementary to corresponding positions in the targeting sequence of the second gRNA.

Embodiment I-59. The SIRV of embodiment I-58, wherein the one or more self-inactivating segments each have between 1 to 3 bases that are not complementary to corresponding positions in the targeting sequence of the second gRNA.

Embodiment I-60. The SIRV of embodiment I-58 or I-59, wherein the base differences of the one or more self-inactivating segments are relative to positions that correspond to positions that are 3′ to the fourth nucleotide of the targeting sequence of the second gRNA when the two sequences are aligned.

Embodiment I-61. The SIRV of any of embodiments I-51 to I-60, wherein the RNP of the Class 2 CRISPR protein and second gRNA exhibit less efficient cleavage of the self-inactivating segment compared to the cleavage of the target nucleic acid of the cell by the RNP of the Class 2 CRISPR protein and first gRNA.

Embodiment I-62. The SIRV of any one of embodiments I-44 to I-61, wherein the third promoter sequence is different from the second promoter sequence and is less efficient at initiating transcription of the second gRNA compared to the second promoter initiating transcription of the first gRNA.

Embodiment I-63. The SIRV of embodiment I-62, wherein the second and the third promoter are selected from the group consisting of U6, mini U6, 5S, Adenovirus 2 (Ad2) VAI, 7SK, H1, bidirectional H1, bidirectional U6, bidirectional 7SK, and bidirectional U6.

Embodiment I-64. The SIRV of embodiment I-62, wherein the second and the third promoter are selected from the group consisting of the sequences of SEQ ID NOS: 494-513, and 2688-2708 as set forth in Table 25, or a sequence at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical or at least about 99% identical thereto.

Embodiment I-65. The SIRV of any one of embodiments I-62 to I-64, wherein the second promoter is U6 and the third promoter is selected from the group consisting of H1, 7SK, and mini U6.

Embodiment I-66. The SIRV of any one of embodiments I-44 to I-65, wherein the Class 2 CRISPR protein further comprises one or more nuclear localization signals (NLS).

Embodiment I-67. The SIRV of embodiment I-66, wherein the one or more NLS are expressed at or near the C-terminus of the CRISPR protein.

Embodiment I-68. The SIRV of embodiment I-66, wherein the one or more NLS are expressed at or near the N-terminus of the CRISPR protein.

Embodiment I-69. The SIRV of embodiment I-66, comprising one or more NLS located at or near the N-terminus and at or near the C-terminus of the CRISPR protein.

Embodiment I-70. The SIRV of any one of embodiments I-66 to I-69, wherein the one or more NLS are selected from the group of sequences consisting of PKKKRKV (SEQ ID NO: 344), KRPAATKKAGQAKKKK (SEQ ID NO: 345), PAAKRVKLD (SEQ ID NO: 346), RQRRNELKRSP (SEQ ID NO: 347), NQSSNFGPMKGGNFGGRSSGPYGGGGQYFAKPRNQGGY (SEQ ID NO: 348), RMRIZFKNKGKDTAELRRRRVEVSVELRKAKKDEQILKRRNV (SEQ ID NO: 349), VSRKRPRP (SEQ ID NO: 350), PPKKARED (SEQ ID NO: 351), PQPKKKPL (SEQ ID NO:352), SALIKKKKKMAP (SEQ ID NO: 353), DRLRR (SEQ ID NO: 354), PKQKKRK (SEQ ID NO: 355), RKLKKKIKKL (SEQ ID NO: 356), REKKKFLKRR (SEQ ID NO: 357), KRKGDEVDGVDEVAKKKSKK (SEQ ID NO: 358), RKCLQAGMNLEARKTKK (SEQ ID NO: 359), PRPRKIPR (SEQ ID NO: 360), PPRKKRTVV (SEQ ID NO: 361), NLSKKKKRKREK (SEQ ID NO: 362), RRPSRPFRKP (SEQ ID NO: 363), KRPRSPSS (SEQ ID NO: 364), KRGINDRNFWRGENERKTR (SEQ ID NO: 365), PRPPKMARYDN (SEQ ID NO: 366), KRSFSKAF (SEQ ID NO: 367), KLKIKRPVK (SEQ ID NO: 368), PKTRRRPRRSQRKRPPT (SEQ ID NO: 370), SRRRKANPTKLSENAKKLAKEVEN (SEQ ID NO: 371), KTRRRPRRSQRKRPPT (SEQ ID NO: 372), RRKKRRPRRKKRR (SEQ ID NO: 373), PKKKSRKPKKKSRK (SEQ ID NO: 374), HKKKHPDASVNFSEFSK (SEQ ID NO: 375), QRPGPYDRPQRPGPYDRP (SEQ ID NO: 376), LSPSLSPLLSPSLSPL (SEQ ID NO: 377), RGKGGKGLGKGGAKRHRK (SEQ ID NO: 378), PKRGRGRPKRGRGR (SEQ ID NO: 379), MSRRRKANPTKLSENAKKLAKEVEN (SEQ ID NO: 598), PKKKRKVPPPPAAKRVKLD (SEQ ID NO: 380), PKKKRKVPPPPKKKRKV (SEQ ID NO: 381), PAKRARRGYKC (SEQ ID NO: 382); KLGPRKATGRW (SEQ ID NO: 383), PRRKREE (SEQ ID NO: 384), PLRKRPRR (SEQ ID NO: 386), PLRKRPRRGSPLRKRPRR (SEQ ID NO: 387), PAAKRVKLDGGKRTADGSEFESPKKKRKV (SEQ ID NO: 388), PAAKRVKLDGGKRTADGSEFESPKKKRKVGIHGVPAA (SEQ ID NO: 389), PAAKRVKLDGGKRTADGSEFESPKKKRKVAEAAAKEAAAKEAAAKA (SEQ ID NO: 390), PAAKRVKLDGGKRTADGSEFESPKKKRKVPG (SEQ ID NO: 391), KRKGSPERGERKRHW (SEQ ID NO: 392), KRTADSQHSTPPKTKRKVEFEPKKKRKV (SEQ ID NO: 393), PKKKRKVGGSKRTADSQHSTPPKTKRKVEFEPKKKRKV (SEQ ID NO: 394), MAPKKKRKVSR (SEQ ID NO: 771), and MAPKKKRKVGGSPKKKRKVGGSPKKKRKVGGSPKKKRKVSR (SEQ ID NO: 772) wherein the one or more NLS are linked to the CRISPR protein or to adjacent NLS with a linker peptide wherein the linker peptide is selected from the group consisting of (G)n (SEQ ID NO: 395), (GS)n (SEQ ID NO: 396), (GSGGS)n (SEQ ID NO: 397), (GGSGGS)n (SEQ ID NO: 398), (GGGS)n (SEQ ID NO: 399), GGSG (SEQ ID NO: 400), GGSGG (SEQ ID NO: 401), GSGSG (SEQ ID NO: 402), GSGGG (SEQ ID NO: 403), GGGSG (SEQ ID NO: 404), GSSSG (SEQ ID NO:405), GPGP (SEQ ID NO: 406), GGP, PPP, PPAPPA (SEQ ID NO: 407), PPPG (SEQ ID NO: 408), PPPGPPP (SEQ ID NO: 409), PPP(GGGS)n (SEQ ID NO: 410), (GGGS)nPPP (SEQ ID NO: 411), AEAAAKEAAAKEAAAKA (SEQ ID NO: 412), wherein the one or more NLS are linked to the CRISPR protein or to adjacent NLS with a linker peptide wherein the linker peptide is selected from the group consisting of RS, (G)n (SEQ ID NO: 395), (GS)n (SEQ ID NO: 396), (GSGGS)n (SEQ ID NO: 397), (GGSGGS)n (SEQ ID NO: 398), (GGGS)n (SEQ ID NO: 399), GGSG (SEQ ID NO: 400), GGSGG (SEQ ID NO: 401), GSGSG (SEQ ID NO: 402), GSGGG (SEQ ID NO: 403), GGGSG (SEQ ID NO: 404), GSSSG (SEQ ID NO:405), GPGP (SEQ ID NO: 406), GGP, PPP, PPAPPA (SEQ ID NO: 407), PPPG (SEQ ID NO: 408), PPPGPPP (SEQ ID NO: 409), PPP(GGGS)n (SEQ ID NO: 410), (GGGS)nPPP (SEQ ID NO: 411), AEAAAKEAAAKEAAAKA (SEQ ID NO: 412), and TPPKTKRKVEFE (SEQ ID NO: 413), where n is 1 to 5.

Embodiment I-71. The SIRV of any one of embodiments I-66 to I-69, wherein the one or more encoded NLS are selected from the group consisting of SEQ ID NOS: 538-597, 599-610, 613, 771-772, 844-846, and 2498-2591, or a sequence having at least 85%, at least 90%, at least 95%, at least 95%, at least 96%, at least 97%, or at least 98% identity thereto.

Embodiment I-72. The SIRV of any one of embodiments I-66 to I-69, wherein the one or more encoded NLS are selected from the group consisting of SEQ ID NOS: 538-597, 599-610, 613, 771-772, 844-846, 2498-2591.

Embodiment I-73. The SIRV of any one of embodiments I-44 to I-72, wherein the CRISPR protein is a CasX protein selected from the group consisting of SEQ ID NOs: 1-3 and 49-321 and 2356-2488, or a sequence having at least about 70%, at least about 80%, at least about 80%, at least about 85%, at least about 90%, or at least about 95%, or at least about 95%, or at least about 96%, or at least about 97%, or at least about 98%, or at least about 99% sequence identity thereto.

Embodiment I-74. The SIRV of any one of embodiments I-44 to I-72, wherein the CRISPR protein is a CasX protein selected from the group consisting of SEQ ID NOs: 1-3 and 49-321 and 2356-2488.

Embodiment I-75. The SIRV of any one of embodiments I-44 to I-72, wherein the Class 2 CRISPR protein is a CasX protein comprising the sequence of SEQ ID NO: 138, or a sequence having at least about 70%, at least about 80%, at least about 80%, at least about 85%, at least about 90%, or at least about 95%, or at least about 95%, or at least about 96%, or at least about 97%, or at least about 98%, or at least about 99% sequence identity thereto.

Embodiment I-76. The SIRV of any one of embodiments I-44 to I-74, wherein the second guide comprises a sequence selected from the group consisting of SEQ ID NO: 2101-2238 and the first guide comprises a sequence selected from the group consisting of SEQ ID NOS: 2276-2296.

Embodiment I-77. The SIRV of embodiment I-75, wherein the second guide comprises the sequence of SEQ ID NO: 2238 and the first guide comprises the sequence of SEQ ID NO: 2296.

Embodiment I-78. The SIRV of any one of embodiments I-44 to I-77, wherein the first and second gRNA each comprise a targeting sequence having 15 nucleotides, 16 nucleotides, 17, nucleotides, 18 nucleotides, 19 nucleotides, or 20 nucleotides.

Embodiment I-79. The SIRV of any one of embodiments I-73 to I-78, wherein the CasX protein is capable of forming a ribonuclear protein complex (RNP) with the first gRNA and the second gRNA upon expression in a cell transduced or transfected with the SIRV.

Embodiment I-80. The SIRV of embodiment I-79, wherein the RNP of the CasX protein and the first gRNA is capable of cleaving the target nucleic acid.

Embodiment I-81. The SIRV of embodiment I-79, wherein the RNP of the CasX protein and the second gRNA is capable of cleaving the self-inactivating segment.

Embodiment I-82. The SIRV of embodiment I-81, wherein the RNP of the CasX protein and the second gRNA exhibit a cleavage rate of the self-inactivating segments that is less efficient compared to the cleavage or rate of cleavage of the target nucleic acid by an RNP of the CasX protein and the first gRNA.

Embodiment I-83. The SIRV of embodiment I-81, wherein the percent cleavage of the self-inactivating segments by the RNP of the CasX protein and the second gRNA is at least about 10%, at least about 20%, at least about 30%, at least about 40%, at least about 50%, at least about 60%, at least about 70%, at least about 80%, or at least about 90% less than the cleavage of the target nucleic acid in a timed in vitro cell-based assay, when assayed under comparable conditions.

Embodiment I-84. The SIRV of embodiment I-81, wherein the time to achieve 90% cleavage of the self-inactivating segments by the RNP of the CasX protein and the second gRNA is delayed, relative to the time to achieve 90% editing of the target nucleic acid by an RNP of the CasX protein and the first gRNA, by at least about 1 day, at least about 2 days, at least about 3 days, at least about 4 days, at least about 5 days, at least about 6 days, at least about 7 days, at least about 8 days, or at least about 9 days, when assayed in an in vitro assay under comparable conditions.

Embodiment I-85. The SIRV of embodiment I-81, wherein cleavage of the self-inactivating segments by the RNP of the CasX protein and the second gRNA has a k_{cleave} rate that is at least about 2-fold, at least about 4-fold, at least about 5-fold, at least about 6-fold, at least about 7-fold, at least about 8-fold, at least about 9-fold, or at least about 10-fold less than the k_{cleave} rate of the target nucleic acid by an RNP of the CasX protein and the first gRNA in an in vitro cell-based assay, when assayed under comparable conditions.

Embodiment I-86. The SIRV of any one of embodiments I-44 to I-85, further comprising at least one accessory element sequence.

Embodiment I-87. The SIRV of embodiment I-86, wherein the accessory element is selected from the group consisting of a poly(A) signal, a gene enhancer element, an intron, a posttranscriptional regulatory element (PTRE), a deaminase, a DNA glycosylase inhibitor, a promoter, a stimulator of CRISPR-mediated homology-directed repair, an activator or repressor of transcription, and a self-inactivating sequence.

Embodiment I-88. The SIRV of embodiment I-87, wherein the PTRE is selected from the group consisting of cytomegalovirus immediate/early intronA, hepatitis B virus PRE (HPRE), Woodchuck Hepatitis virus PRE (WPRE), and 5′ untranslated segment (UTR) of human heat shock protein 70 mRNA (Hsp70).

Embodiment I-89. The SIRV of any one of embodiments I-86 to I-88, wherein the accessory element(s) enhance the expression, binding, activity, or performance of the CRISPR protein in the transduced or transfected cell as compared to the CRISPR protein in the absence of said accessory element.

Embodiment I-90. The SIRV of embodiment I-89, wherein the enhancement is an increase in editing of a target nucleic acid in a timed in vitro assay of at least about 10%, at least about 20%, at least about 30%, at least about 40%, at least about 50%, at least about 60%, at least about 70%, at least about 80%, at least about 90%, at least about 100%, at least about 150%, at least about 200%, or at least about 300%.

Embodiment I-91. The SIRV of any one of embodiments I-44 to I-90, wherein the packaging element is selected from the group consisting of AAV 5′ and 3′ inverted terminal repeats (ITR), adenovirus packaging protein, lentiviral psi packaging element, and gammaretroviral psi packaging element.

Embodiment I-92. The SIRV of embodiment I-91, wherein the AAV 5′ and 3′ ITRs are derived from serotype AAV1, AAV2, AAV3, AAV4, AAV5, AAV6, AAV7, AAV8, AAV9, AAV10, AAV11, AAV12, AAV 44.9, AAV 9.45, AAV 9.61, AAV-Rh74, AAVRh10, or a chimeric combination thereof.

Embodiment I-93. The SIRV of embodiment I-92, wherein the ITRs are derived from serotype AAV2.

Embodiment I-94. A SIRV comprising a polynucleotide comprising one or more components selected from:
a) one or more packaging components;
b) a sequence encoding a Class 2 CRISPR protein;
c) a first promoter operably linked to the sequence encoding the Class 2 CRISPR protein;
d) a sequence encoding a first guide RNA (gRNA) scaffold and a targeting sequence that is complementary to a target nucleic acid of a cell to be modified;
e) a second promoter sequence operably linked to the sequence encoding the first gRNA;
f) a sequence encoding a second guide RNA (gRNA) and a targeting sequence complementary to one or more self-inactivating segments of the SIRV;
g) a third promoter sequence operably linked to the sequence encoding the second gRNA, wherein the third promoter has a sequence different from the sequence of the second promoter; and
h) one or more self-inactivating segments of the polynucleotide comprising a protospacer adjacent motif (PAM) sequence and a polynucleotide sequence capable of being bound and cleaved by a ribonuclear protein complex (RNP) of the Class 2 CRISPR protein and the second gRNA.

Embodiment I-95. The SIRV of embodiment I-94, comprising components (a)-(h).

Embodiment I-96. The SIRV of embodiment I-94 or I-95, wherein the third promoter is less efficient at initiating transcription of the second gRNA compared to the ability of the second promoter to initiate transcription of the first gRNA.

Embodiment I-97. The SIRV of any one of embodiments I-94 to I-96, wherein the one or more self-inactivating segments of the polynucleotide are located at a position:
a) 5′ or 3′ adjacent to or within the sequence encoding the Class 2 CRISPR protein;
b) 5′ or 3′ adjacent to or within a Kozak sequence located between the first promoter and the sequence encoding the Class 2 CRISPR protein;
c) 5′ or 3′ adjacent to or within to the first promoter sequence;
d) 5′ or 3′ adjacent to or within the second promoter sequence;
e) downstream of the transcriptional start site for the sequence encoding the Class 2 CRISPR protein;
f) within one or more inserted introns in the polynucleotide encoding the Class 2 CRISPR protein;
g) at the 3′ end of the polynucleotide encoding the Class 2 CRISPR protein, between the stop codon and poly(A) termination site; or
h) any combination of (a)-(g).

Embodiment I-98. The SIRV of embodiment I-97, wherein the self-inactivating segment comprises any 15-21 nucleotide sequence portion of the positions of embodiment I-97 that is 3′ adjacent to a PAM sequence recognized by an RNP of the Class 2 CRISPR protein and the second gRNA.

Embodiment I-99. The SIRV of any one of embodiments I-94 to I-98, wherein the second and the third promoters are independently selected from the group consisting of U6, mini U6, 5S, Adenovirus 2 (Ad2) VAI, 7SK, H1, bidirectional H1, bidirectional U6, bidirectional 7SK, and bidirectional U6.

Embodiment I-100. The SIRV of any one of embodiments I-94 to I-99, wherein the second and the third promoters are selected from the group consisting of the sequences of SEQ ID NOS: 494-513, and 2688-2708, or a sequence having at least about 80% identical, at least about 85% identical, at least about 90% identical, at least about 91% identical, at least about 92% identical, at least about 93% identical, at least about 94% identical, at least about 95% identical, at least about 96% identical, at least about 97% identical, at least about 98% identical or at least about 99% identical thereto.

Embodiment I-101. The SIRV of any one of embodiments I-94 to I-99, wherein the second and the third promoters are selected from the group consisting of the sequences of SEQ ID NOS: 494-513 and 2688-2708.

Embodiment I-102. The SIRV of any one of embodiments I-99 to I-101, wherein the second promoter is U6 and the third promoter is selected from the group consisting of H1, 7SK, and mini U6.

Embodiment I-103. The SIRV of any one of embodiments I-94 to I-102, wherein cleavage of the self-inactivating segments of the polynucleotide in a cell transduced or transfected with the SIRV by the RNP of the Class 2 CRISPR protein and the second gRNA results in reduced or eliminated expression of the Class 2 CRISPR protein or the gRNA encoded by the polynucleotide.

Embodiment I-104. The SIRV of any one of embodiments I-94 to I-103, wherein the PAM sequence of the one or more self-inactivating segments:
a) is identical to the PAM sequence of the target nucleic acid of the cell to be modified; and
b) promotes less efficient cleavage or rate of cleavage of the self-inactivating segment by the RNP compared to the PAM sequence 5′ and adjacent to the target nucleic acid of the cell to be modified.

Embodiment I-105. The SIRV of embodiment I-104, wherein the PAM sequence of the target nucleic acid is NTN.

Embodiment I-106. The SIRV of any one of embodiments I-94 to I-105, wherein the PAM sequence of the one or more self-inactivating segments:
a) is different from the PAM sequence of the target nucleic acid of the cell to be modified; and
b) promotes less efficient cleavage or cleavage rate of the self-inactivating segment by the RNP compared to the PAM of the target nucleic acid of the cell.

Embodiment I-107. The SIRV of embodiment I-106, wherein:
a) if the PAM sequence of the target nucleic acid of the cell to be modified is TTC and the PAM preference of the Class 2 CRISPR protein is TTC, then the PAM sequence of the one or more self-inactivating segments is selected from the group consisting of ATC, CTC, TTT, GTT, and GTC;
b) if the PAM sequence of the target nucleic acid of the cell to be modified is ATC and the PAM preference of the Class 2 CRISPR protein is TTC, then the PAM sequence of the one or more self-inactivating segments is selected from the group consisting of CTC, TTT, GTT, and GTC; or
c) if the PAM sequence of the target nucleic acid of the cell to be modified is CTC and the PAM preference of the Class 2 CRISPR protein is TTC, then the PAM sequence of the one or more self-inactivating segments is GTC, TTT, or GTT.

Embodiment I-108. The SIRV of embodiment I-106, wherein:
a) if the PAM sequence of the target nucleic acid of the cell to be modified is ATC and the PAM preference of the Class 2 CRISPR protein is ATC, then the PAM sequence of the one or more self-inactivating segments is selected from the group consisting of TTC, CTC, TTT, GTT, and GTC;
b) if the PAM sequence of the target nucleic acid of the cell to be modified is CTC and the PAM preference of the Class 2 CRISPR protein is ATC, then the PAM sequence of the one or more self-inactivating segments is selected from the group consisting of TTC, TTT, GTT, and GTC; or
c) if the PAM sequence of the target nucleic acid of the cell to be modified is GTC and the PAM preference of the Class 2 CRISPR protein is ATC, then the PAM sequence of the one or more self-inactivating segments is TTC.

Embodiment I-109. The SIRV of embodiment I-106, wherein:
a) if the PAM sequence of the target nucleic acid of the cell to be modified is GTC and the PAM preference of the Class 2 CRISPR protein is GTC, then the PAM sequence of the one or more self-inactivating segments is selected from the group consisting of ATC, CTC, TTT, GTT, and TTC.
b) if the PAM sequence of the target nucleic acid of the cell to be modified is GTC and the PAM preference of the Class 2 CRISPR protein is ATC, then the PAM sequence of the one or more self-inactivating segments is selected from the group consisting of TTC, TTT, GTT, and CTC; or
c) if the PAM sequence of the target nucleic acid of the cell to be modified is CTC and the PAM preference of the Class 2 CRISPR protein is GTC, then the PAM sequence of the one or more self-inactivating segments is TTC, TTT, or GTT.

Embodiment I-110. The SIRV of embodiment I-106, wherein:
a) if the PAM sequence of the target nucleic acid of the cell to be modified is CTC and the PAM preference of the Class 2 CRISPR protein is CTC, then the PAM sequence of the one or more self-inactivating segments is selected from the group consisting of ATC, GTC, TTT, GTT, and TTC.
b) if the PAM sequence of the target nucleic acid of the cell to be modified is ATC and the PAM preference of the Class 2 CRISPR protein is CTC, then the PAM sequence of the one or more self-inactivating segments is selected from the group consisting of GTC, TTT, GTT, and TTC; or
c) if the PAM sequence of the target nucleic acid of the cell to be modified is GTC and the PAM preference of the Class 2 CRISPR protein is CTC, then the PAM sequence of the one or more self-inactivating segments is TTC, TTT, or GTT.

Embodiment I-111. The SIRV of any one of embodiments I-94 to I-110, wherein the one or more self-inactivating segment sequences:
a) each have between 1 to 5 bases that are not complementary to corresponding positions in the targeting sequence of the second gRNA; and
b) exhibit less efficient cleavage of the self-inactivating segment by the RNP of the Class 2 CRISPR protein and second gRNA compared to the cleavage of the target nucleic acid of the cell by the RNP of the Class 2 CRISPR protein and first gRNA.

Embodiment I-112. The SIRV of embodiment I-111, wherein the one or more self-inactivating segments each have between 1 to 3 bases that are not complementary to corresponding positions in the targeting sequence of the second gRNA.

Embodiment I-113. The SIRV of embodiments I-111 and I-112, wherein the base differences of the one or more self-inactivating segments are relative to positions that correspond to positions that are 3′ to the fourth nucleotide of the targeting sequence of the first gRNA when the two sequences are aligned.

Embodiment I-114. The SIRV of any one of embodiments I-101 to I-113, wherein the cleavage of the self-inactivating segments is at least about 10%, at least about 20%, at least about 30%, at least about 40%, at least about 50%, at least about 60%, at least about 70%, at least about 80%, or at least about 90% less than the cleavage of the target nucleic acid in a timed in vitro cell-based assay, when assayed under comparable conditions.

Embodiment I-115. The SIRV of any one of embodiments I-101 to I-113, wherein the cleavage of the self-inactivating segments in a cell transduced or transfected with the SIRV by the RNP to achieve 90% cleavage is delayed, relative to the time to achieve 90% editing of the target nucleic acid in the cell by at least about 1 day, at least about 2 days, at least about 3 days, at least about 4 days, at least about 5 days, at least about 6 days, at least about 7 days, at least about 8 days, or at least about 9 days, when assayed in an in vitro assay under comparable conditions.

Embodiment I-116. The SIRV of any one of embodiments I-101 to I-113, wherein cleavage of the self-inactivating segments in a cell transduced or transfected with the SIRV by the RNP has a k_{cleave} rate that is at least about 2-fold, at least about 4-fold, at least about 5-fold, at least about 6-fold, at least about 7-fold, at least about 8-fold, at least about 9-fold, or at least about 10-fold less than the k_{cleave} rate of the target nucleic acid in an in vitro cell-based assay, when assayed under comparable conditions.

Embodiment I-117. The SIRV of any one of embodiments I-94 to I-116, wherein the Class 2 CRISPR protein further comprises one or more nuclear localization signals (NLS).

Embodiment I-118. The SIRV of embodiment I-117, wherein the one or more NLS are expressed at or near the C-terminus of the CRISPR protein.

Embodiment I-119. The SIRV of embodiment I-117, wherein the one or more NLS are expressed at or near the N-terminus of the CRISPR protein.

Embodiment I-120. The SIRV of embodiment I-117, comprising one or more NLS located at or near the N-terminus and at or near the C-terminus of the CRISPR protein.

Embodiment I-121. The SIRV of any one of embodiments I-117 to I-120, wherein the one or more NLS are selected from the group of sequences consisting of PKKKRKV (SEQ ID NO: 344), KRPAATKKAGQAKKKK (SEQ ID NO: 345), PAAKRVKLD (SEQ ID NO: 346), RQRRNELKRSP (SEQ ID NO: 347), NQSSNFGPMKGGNFGGRSSGPYGGGGQYFAKPRNQGGY (SEQ ID NO: 348), RMRIZFKNKGKDTAELRRRRVEVSVELRKAKKDEQILKRRNV (SEQ ID NO: 349), VSRKRPRP (SEQ ID NO: 350), PPKKARED (SEQ ID NO: 351), PQPKKKPL (SEQ ID NO:352), SALIKKKKKMAP (SEQ ID NO: 353), DRLRR (SEQ ID NO: 354), PKQKKRK (SEQ ID NO: 355), RKLKKKIKKL (SEQ ID NO: 356), REKKKFLKRR (SEQ ID NO: 357), KRKGDEVDGVDEVAKKKSKK (SEQ ID NO: 358), RKCLQAGMNLEARKTKK (SEQ ID NO: 359), PRPRKIPR (SEQ ID NO: 360), PPRKKRTVV (SEQ ID NO: 361), NLSKKKKRKREK (SEQ ID NO: 362), RRPSRPFRKP (SEQ ID NO: 363), KRPRSPSS (SEQ ID NO: 364), KRGINDRNFWRGENERKTR (SEQ ID NO: 365), PRPPKMARYDN (SEQ ID NO: 366), KRSFSKAF (SEQ ID NO: 367), KLKIKRPVK (SEQ ID NO: 368), PKTRRRPRRSQRKRPPT (SEQ ID NO: 370), SRRRKANPTKLSENAKKLAKEVEN (SEQ ID NO: 371), KTRRRPRRSQRKRPPT (SEQ ID NO: 372), RRKKRRPRRKKRR (SEQ ID NO: 373), PKKKSRKPKKKSRK (SEQ ID NO: 374), HKKKHPDASVNFSEFSK (SEQ ID NO: 375), QRPGPYDRPQRPGPYDRP (SEQ ID NO: 376), LSPSLSPLLSPSLSPL (SEQ ID NO: 377), RGKGGKGLGKGGAKRHRK (SEQ ID NO: 378), PKRGRGRPKRGRGR (SEQ ID NO: 379), MSRRRKANPTKLSENAKKLAKEVEN (SEQ ID NO: 598), PKKKRKVPPPPAAKRVKLD (SEQ ID NO: 380), PKKKRKVPPPPKKKRKV (SEQ ID NO: 381), PAKRARRGYKC (SEQ ID NO: 382); KLGPRKATGRW (SEQ ID NO: 383), PRRKREE (SEQ ID NO: 384), PLRKRPRR (SEQ ID NO: 386), PLRKRPRRGSPLRKRPRR (SEQ ID NO: 387), PAAKRVKLDGGKRTADGSEFESPKKKRKV (SEQ ID NO: 388), PAAKRVKLDGGKRTADGSEFESPKKKRKVGIHGVPAA (SEQ ID NO: 389), PAAKRVKLDGGKRTADGSEFESPKKKRKVAEAAAKEAAAKEAAAKA (SEQ ID NO: 390), PAAKRVKLDGGKRTADGSEFESPKKKRKVPG (SEQ ID NO: 391), KRKGSPERGERKRHW (SEQ ID NO: 392), KRTADSQHSTPPKTKRKVEFEPKKKRKV (SEQ ID NO: 393), PKKKRKVGGSKRTADSQHSTPPKTKRKVEFEPKKKRKV (SEQ ID NO: 394), MAPKKKRKVSR (SEQ ID NO: 771), and MAPKKKRKVGGSPKKKRKVGGSPKKKRKVGGSPKKKRKVSR (SEQ ID NO: 772) wherein the one or more NLS are linked to the CRISPR protein or to adjacent NLS with a linker peptide wherein the linker peptide is selected from the group consisting of RS, (G)n (SEQ ID NO: 395), (GS)n (SEQ ID NO: 396), (GSGGS)n (SEQ ID NO: 397), (GGSGGS)n (SEQ ID NO: 398), (GGGS)n (SEQ ID NO: 399), GGSG (SEQ ID NO: 400), GGSGG (SEQ ID NO: 401), GSGSG (SEQ ID NO: 402), GSGGG (SEQ ID NO: 403), GGGSG (SEQ ID NO: 404), GSSSG (SEQ ID NO:405), GPGP (SEQ ID NO: 406), GGP, PPP, PPAPPA (SEQ ID NO: 407), PPPG (SEQ ID NO: 408), PPPGPPP (SEQ ID NO: 409), PPP(GGGS)n (SEQ ID NO: 410), (GGGS)nPPP (SEQ ID NO: 411), AEAAAKEAAAKEAAAKA (SEQ ID NO: 412), wherein the one or more NLS are linked to the CRISPR protein or to adjacent NLS with a linker peptide wherein the linker peptide is selected from the group consisting of (G)n (SEQ ID NO: 395), (GS)n (SEQ ID NO: 396), (GSGGS)n (SEQ ID NO: 397), (GGSGGS)n (SEQ ID NO: 398), (GGGS)n (SEQ ID NO: 399), GGSG (SEQ ID NO: 400), GGSGG (SEQ ID NO: 401), GSGSG (SEQ ID NO: 402), GSGGG (SEQ ID NO: 403), GGGSG (SEQ ID NO: 404), GSSSG (SEQ ID NO:405), GPGP (SEQ ID NO: 406), GGP, PPP, PPAPPA (SEQ ID NO: 407), PPPG (SEQ ID NO: 408), PPPGPPP (SEQ ID NO: 409), PPP(GGGS)n (SEQ ID NO: 410), (GGGS)nPPP (SEQ ID NO: 411), AEAAAKEAAAKEAAAKA (SEQ ID NO: 412), and TPPKTKRKVEFE (SEQ ID NO: 413), where n is 1 to 5.

Embodiment I-122. The SIRV of any one of embodiments I-117 to I-120, wherein the one or more encoded NLS are selected from the group consisting of SEQ ID NOS: 538-597, 599-610, 613, 771-772, 844-846, and 2498-2591 or a sequence having at least 85%, at least 90%, at least 95%, at least 95%, at least 96%, at least 97%, at least 98% identity thereto.

Embodiment I-123. The SIRV of any one of embodiments I-117 to I-120, wherein the one or more encoded NLS are selected from the group consisting of SEQ ID NOS: 538-597, 599-610, 613, 771-772, 844-846, and 2498-2591.

Embodiment I-124. The SIRV of any one of embodiments I-94 to I-123, wherein the CRISPR protein is a CasX protein selected from the group of sequences of SEQ ID NOs: 1-3, 49-321 and 2356-2488, or a sequence having at least about 70%, at least about 80%, at least about 90%, or at least about 95%, or at least about 95%, or at least about 96%, or at least about 97%, or at least about 98%, or at least about 99% sequence identity thereto.

Embodiment I-125. The SIRV of any one of embodiments I-94 to I-123, wherein the CRISPR protein is a CasX protein selected from the group of sequences of SEQ ID NOs: 1-3, 49-321 and 2356-2488.

Embodiment I-126. The SIRV of any one of embodiments I-94 to I-124, wherein the Class 2 CRISPR protein is a CasX protein comprising the sequence of SEQ ID NO: 138, or a sequence having at least about 70%, at least about 80%, at least about 80%, at least about 85%, at least about 90%, or at least about 95%, or at least about 95%, or at least about 96%, or at least about 97%, or at least about 98%, or at least about 99% sequence identity thereto.

Embodiment I-127. The SIRV of any one of embodiments I-94 to I-125, wherein the first gRNA has a scaffold comprising a sequence selected from the group of sequences of SEQ ID NOS: 2101-2331 and 3992-3995, or a sequence having at least about 70%, at least about 80%, at least about 90%, at least about 95%, at least about 96%, at least about 97%, at least about 98%, at least about 99%, or 100% sequence identity thereto.

Embodiment I-128. The SIRV of any one of embodiments I-94 to I-125, wherein the first gRNA has a scaffold comprising a sequence selected from the group of sequences of SEQ ID NOS: 2101-2331 and 3992-3995.

Embodiment I-129. The SIRV of any one of embodiments I-94 to I-128, wherein the second gRNA has a scaffold comprising a sequence selected from the group of sequences of SEQ ID NOS: 2101-2331 and 3992-3995, or a sequence having at least about 70%, at least about 80%, at least about 90%, at least about 95%, at least about 96%, at least about 97%, at least about 98%, at least about 99%, or 100% sequence identity thereto.

Embodiment I-130. The SIRV of any one of embodiments I-94 to I-128, wherein the second gRNA has a scaffold comprising a sequence selected from the group consisting of sequences SEQ ID NOS: 2101-2331 and 3992-3995.

Embodiment I-131. The SIRV of any one of embodiments I-94 to I-130, wherein the second guide comprises a sequence selected from the group consisting of SEQ ID NO: 2101-2238 and the first guide comprises a sequence selected from the group consisting of SEQ ID NOS: 2276-2296.

Embodiment I-132. The SIRV of embodiment I-131, wherein the second guide comprises the sequence of SEQ ID NO: 2238 and the first guide comprises the sequence of SEQ ID NO: 2296.

Embodiment I-133. The SIRV of any one of embodiments I-94 to I-132, wherein the first and second gRNA each comprise a targeting sequence having 15 nucleotides, 16 nucleotides, 17, nucleotides, 18 nucleotides, 19 nucleotides, or 20 nucleotides.

Embodiment I-134. The SIRV of any one of embodiments I-94 to I-133, wherein upon expression in a transduced or transfected cell, the CasX protein is capable of forming a ribonuclear protein complex (RNP) with the first gRNA and the second gRNA upon expression in the transduced or transfected cell.

Embodiment I-135. The SIRV of embodiment I-134, wherein the RNP comprising the first gRNA exhibits, upon binding to the target nucleic acid sequence in an in vitro editing assay, an improved characteristic as compared to an RNP comprising the second gRNA upon binding to its respective target nucleic acid sequence.

Embodiment I-136. The SIRV of embodiment I-135, wherein the improved characteristic is selected from the group consisting of increased percentage of cleavage-competent conformation, increased cleavage rate, and increased initial cleavage velocity.

Embodiment I-137. The SIRV of any one of embodiments I-94 to I-136, further comprising at least one accessory element sequence.

Embodiment I-138. The SIRV of embodiment I-137, wherein the accessory element is selected from the group consisting of a poly(A) signal, a gene enhancer element, an intron, a posttranscriptional regulatory element (PTRE), a deaminase, a DNA glycosylase inhibitor, a promoter, a stimulator of CRISPR-mediated homology-directed repair, an activator or repressor of transcription, and a self-cleaving sequence.

Embodiment I-139. The SIRV of embodiment I-138, wherein the PTRE is selected from the group consisting of cytomegalovirus immediate/early intronA, hepatitis B virus PRE (HPRE), Woodchuck Hepatitis virus PRE (WPRE), and 5′ untranslated segment (UTR) of human heat shock protein 70 mRNA (Hsp70).

Embodiment I-140. The SIRV of any one of embodiments I-137 to I-139, wherein the accessory element(s) enhance the expression, binding, activity, or performance of the CRISPR protein in the cell as compared to the CRISPR protein in the absence of said accessory element.

Embodiment I-141. The SIRV of embodiment I-140, wherein the enhancement is an increase in editing of the target nucleic acid in a timed in vitro assay of at least about 10%, at least about 20%, at least about 30%, at least about 40%, at least about 50%, at least about 60%, at least about 70%, at least about 80%, at least about 90%, at least about 100%, at least about 150%, at least about 200%, or at least about 300%.

Embodiment I-142. The SIRV of any one of embodiments I-94 to I-141, wherein the packaging element is selected from the group consisting of AAV 5′ and 3′ inverted terminal repeats (ITR), adenovirus packaging protein, lentiviral psi packaging element, and gammaretroviral psi packaging element.

Embodiment I-143. The SIRV of embodiment I-142, wherein the AAV 5′ and 3′ ITRs are derived from serotype AAV1, AAV2, AAV3, AAV4, AAV5, AAV6, AAV7, AAV8, AAV9, AAV10, AAV11, AAV12, AAV 44.9, AAV 9.45, AAV 9.61, AAV-Rh74, or AAVRh10.

Embodiment I-144. The SIRV of embodiment I-143, wherein the ITRs are derived from serotype AAV2.

Embodiment I-145. The SIRV of any one of embodiments I-1 to I-144, wherein the polynucleotide comprises a sequence selected from the group consisting of SEQ ID NOs 4151-4156, or a sequence having at least about 70%, at least about 80%, at least about 80%, at least about 85%, at least about 90%, or at least about 95%, or at least about 95%, or at least about 96%, or at least about 97%, or at least about 98%, or at least about 99% sequence identity thereto.

Embodiment I-146. The SIRV of any one of embodiments I-1 to I-144, wherein the polynucleotide comprises or encodes one or more components in which the sequence comprises less than about 10%, less than about 5%, or less than about 1% CpG dinucleotides.

Embodiment I-147. The SIRV of embodiment I-146, wherein the components are selected from the group consisting of 5′ ITR, 3′ ITR, Pol III promoter, Pol II promoter, encoding sequence for CRISPR nuclease, encoding sequence for gRNA, accessory element, and poly(A) signal.

Embodiment I-148. A self-inactivating viral-derived particle comprising
a) a viral capsid; and
b) the SIRV of any one of embodiments I-1 to I-147.

Embodiment I-149. The self-inactivating viral-derived particle of embodiment I-148, wherein the viral capsid is derived from an adeno associated virus (AAV), an adenovirus, a lentivirus, or a gammaretrovirus.

Embodiment I-150. The self-inactivating viral-derived particle of embodiment I-149, wherein the capsid is derived from an AAV serotype selected from the group consisting of AAV1, AAV2, AAV3, AAV4, AAV5, AAV6, AAV7, AAV8, AAV9, AAV10, AAV11, AAV12, AAV 44.9, AAV 9.45, AAV 9.61, AAV-Rh74, AAVRh10, and chimeras thereof.

Embodiment I-151. The self-inactivating viral-derived particle of embodiment I-150, wherein the SIRV packaging component is 5′ and 3′ AAV ITR selected from the same serotype as the AAV capsid.

Embodiment I-152. The self-inactivating viral-derived particle of embodiment I-150, wherein the SIRV packaging component is 5′ and 3′ AAV ITR selected from a different serotype as the AAV capsid.

Embodiment I-153. The self-inactivating viral-derived particle of embodiment I-150, wherein the SIRV packaging component is 5′ and 3′ AAV ITR selected from serotype AAV2.

Embodiment I-154. A pharmaceutical composition, comprising the self-inactivating viral-derived particle of any one of embodiments I-148 to I-153, and a pharmaceutically acceptable carrier, diluent or excipient.

Embodiment I-155. A method of modifying a target nucleic acid in a cell, comprising transfecting the cell with the SIRV of any one of embodiments I-1 to I-147, wherein the target nucleic acid is modified by an RNP of the expressed Class 2 CRISPR protein and the first gRNA.

Embodiment I-156. The method of embodiment I-155, wherein the modifying comprises introducing a single-stranded break in the target nucleic acid sequence of the cell.

Embodiment I-157. The method of embodiment I-155, wherein the modifying comprises introducing a double-stranded break in the target nucleic acid sequence of the cell.

Embodiment I-158. The method of any one of embodiments I-155 to I-157, wherein the self-inactivating segment is cleaved by an RNP of the Class 2 CRISPR protein and the first gRNA subsequent to the modifying of the target nucleic acid of the cell.

Embodiment I-159. The method of any one of embodiments I-155 to I-157, wherein the self-inactivating segment is cleaved by an RNP of the Class 2 CRISPR protein and the second gRNA subsequent to the modifying of the target nucleic acid.

Embodiment I-160. The method of embodiment I-158 or I-159, wherein the self-inactivating segment is cleaved at least 1 day, at least 2 days, at least 3 days, at least 4 days, at least 5 days, at least 6 days, or at least 7 days after the modifying of the target nucleic acid.

Embodiment I-161. The method of any one of embodiments I-155 to I-159, wherein the cleavage of the self-inactivating segment results in reduced off-target modifying of nucleic acid in the cell compared to a cell transduced with an SIRV not comprising the self-inactivating segments.

Embodiment I-162. The method of any one of embodiments I-155 to I-159, wherein the cleavage of the self-inactivating segment results in reduced or eliminated expression of the Class 2 CRISPR protein in the cell.

Embodiment I-163. A method of modifying a target nucleic acid in a population of cells of a subject, comprising administering a therapeutic dose of the self-inactivating viral-derived particle of any one of embodiments I-148 to I-153 to the subject, wherein the target nucleic acid of the cells transduced is modified by an RNP of the Class 2 CRISPR protein and the gRNA expressed in the cells.

Embodiment I-164. The method of embodiment I-163, wherein the self-inactivating viral-derived particle is administered to the subject at a dose of at least about 1×10⁵ vector genomes/kg (vg/kg), at least about 1×10⁶ vg/kg, at least about 1×10⁷ vg/kg, at least about 1×10⁸ vg/kg, at least about 1×10⁹ vg/kg, at least about 1×10¹⁰ vg/kg, at least about 1×10¹¹ vg/kg, at least about 1×10¹² vg/kg, at least about 1×10¹³ vg/kg, at least about 1×10¹⁴ vg/kg, at least about 1×10¹⁵ vg/kg, or at least about 1×10¹⁶ vg/kg.

Embodiment I-165. The method of embodiment I-163, wherein the self-inactivating viral-derived particle is administered to the subject at a dose of at least about 1×10⁵ vg/kg to about 1×10¹⁶ vg/kg, at least about 1×10⁶ vg/kg to about 1×10¹⁵ vg/kg, or at least about 1×10⁷ vg/kg to about 1×10¹⁴ vg/kg.

Embodiment I-166. The method of any one of embodiments I-163 to I-165, wherein the subject is selected from the group consisting of rodent, mouse, rat, and non-human primate.

Embodiment I-167. The method of any one of embodiments I-163 to I-165, wherein the subject is a human.

Embodiment I-168. The method of any one of embodiments I-163 to I-167, wherein the self-inactivating viral-derived particle is administered to the subject according to a treatment regimen comprising one or more consecutive doses using a therapeutically effective dose of the self-inactivating viral derived particle.

Embodiment I-169. The method of any one of embodiments I-163 to I-168, wherein the therapeutically effective dose is administered to the subject as two or more doses over a period of at least two weeks, or at least one month, or at least two months, or at least three months, or at least four months, or at least five months, or at least six months, or once a year, or every 2 or 3 years.

Embodiment I-170. The method of any one of embodiments I-163 to I-169, wherein the therapeutically effective dose is administered by a route of administration selected from the group consisting of subcutaneous, intradermal, intraneural, intranodal, intramedullary, intramuscular, intravenous, intracerebroventricular, intracisternal, intrathecal, intracranial, intralumbar, intratracheal, intraosseous, inhalatory, intracontralateral striatum, intraocular, intravitreal, intralymphatical, intraperitoneal routes and sub-retinal routes, wherein the administering method is injection, transfusion, or implantation.

Embodiment I-171. The method of any one of embodiments I-163 to I-170, wherein the modifying comprises introducing a single-stranded break in the target nucleic acid sequence of the cells of the subject.

Embodiment I-172. The method of any one of embodiments I-163 to I-170, wherein the modifying comprises introducing a double-stranded break in the target nucleic acid sequence of the cells of the subject.

Embodiment I-173. The method of embodiment I-171 or I-172, wherein the modifying results in an insertion, deletion, or mutation in the target nucleic acid sequence of the cells of the subject.

Embodiment I-174. The method of any one of embodiments I-163 to I-173, wherein the self-inactivating segment is cleaved by an RNP of the Class 2 CRISPR protein and the first gRNA subsequent to the modifying of the target nucleic acid of the cells of the subject.

Embodiment I-175. The method of any one of embodiments I-163 to I-173, wherein the self-inactivating segment is cleaved by an RNP of the Class 2 CRISPR protein and the second gRNA subsequent to the modifying of the target nucleic acid of the cells of the subject.

Embodiment I-176. The method of embodiment I-174 or I-175, wherein the self-inactivating segment is cleaved at least 1 day, at least 2 days, at least 3 days, at least 4 days, at least 5 days, at least 6 days, at least 7 days, at least 8 days, or at least 9 days after the modifying of the target nucleic acid of the cells of the subject.

Embodiment I-177. The method of any one of embodiments I-174 to I-176, wherein the cleavage of the self-inactivating segment results in reduced or eliminated expression of the Class 2 CRISPR protein in the cell of the cells of the subject.

Embodiment I-178. The method of any one of embodiments I-174 to I-177, wherein the cleavage of the self-inactivating segment results in reduced off-target modifying of nucleic acid in the cells compared to cells transduced with an AAV not comprising the self-inactivating segments.

Embodiment I-179. A composition comprising:
a) an AAV expression cassette; and
b) a polynucleotide comprising sequences encoding one or more small hairpin RNA (shRNA) sequences, each operably linked to a promoter.

Embodiment I-180. The composition of embodiment I-179, wherein the AAV expression cassette comprises
a) a first adeno-associated virus (AAV) inverted terminal repeat (ITR) sequence;
b) a second AAV ITR sequence;
c) a sequence encoding a Class 2 CRISPR protein having a single RNA-guided RuvC domain;
d) a first promoter operably linked to the sequence encoding the Class 2 CRISPR protein;
e) a sequence encoding a first guide RNA (gRNA) comprising a scaffold sequence and a linked targeting sequence that is complementary to and capable of hybridizing with a target nucleic acid of a cell to be modified; and
f) a second promoter sequence operably linked to the sequence encoding the first gRNA.

Embodiment I-181. The composition of embodiment I-179 or I-180, wherein the polynucleotide comprises an encoding sequence for a single shRNA and linked promoter.

Embodiment I-182. The composition of embodiment I-179 or I-180, wherein the polynucleotide comprises an encoding sequence for two shRNA and linked promoters.

Embodiment I-183. The composition of embodiment I-179 or I-180, wherein the polynucleotide comprises an encoding sequence for three shRNA and linked promoters.

Embodiment I-184. The composition of any one of embodiments I-179 to I-183, wherein the shRNA encoding sequence comprises a sequence selected from the group consisting of SEQ ID NOS: 2640-2687, or a sequence having at least 85%, at least 90%, at least 95%, at least 95%, at least 96%, at least 97%, at least 98% identity thereto.

Embodiment I-185. The composition of any one of embodiments I-179 to I-183, wherein the shRNA encoding sequence comprises a sequence selected from the group consisting of SEQ ID NOS: 2640-2687.

Embodiment I-186. The composition of any one of embodiments I-179 to I-185, wherein the polynucleotide comprising the shRNA and linked promoters are linked exterior to the AAV transgene inserted into a bacterial plasmid backbone.

Embodiment I-187. The composition of any one of embodiments I-179 to I-185, wherein the polynucleotide comprising the shRNA and linked promoters are inserted into
a) an AAV RepCap plasmid;
b) an AAV Helper plasmid; and/or
c) a separate vector.

Embodiment I-188. The composition of any one of embodiments I-180 to I-187, wherein the encoded Class 2, CRISPR protein is a CasX.

Embodiment I-189. The composition of embodiment I-188, wherein the encoded CasX comprises a sequence selected from the group consisting of SEQ ID NOS: 1-3, 49-321 and 2356-2488, or a sequence having at least 85%, at least 90%, at least 95%, at least 95%, at least 96%, at least 97%, at least 98%, or at least 99% identity thereto.

Embodiment I-190. The composition of embodiment I-188, wherein the encoded CasX comprises a sequence selected from the group consisting of the sequences of SEQ ID NOS: 1-3, 49-321 and 2356-2488.

Embodiment I-191. The composition of embodiment I-188, wherein the encoded Class 2 CRISPR protein is a CasX protein comprising the sequence of SEQ ID NO: 138, or a sequence having at least about 70%, at least about 80%, at least about 80%, at least about 85%, at least about 90%, or at least about 95%, or at least about 95%, or at least about 96%, or at least about 97%, or at least about 98%, or at least about 99% sequence identity thereto.

Embodiment I-192. The composition of any one of embodiments I-180 to I-191, wherein the gRNA has a scaffold comprising a sequence selected from the group of sequences consisting of SEQ ID NOS: 2101-2331 and 3992-3995, or a sequence having at least about 70%, at least about 80%, at least about 80%, at least about 85%, at least about 90%, or at least about 95%, or at least about 95%, or at least about 96%, or at least about 97%, or at least about 98%, or at least about 99% sequence identity thereto.

Embodiment I-193. The composition of any one of embodiments I-188 to I-192, wherein the CasX and gRNA encoding sequences are capable of being transcribed in a packaging cell transfected with the AAV expression cassette.

Embodiment I-194. The composition of embodiment I-192, wherein the AAV expression cassette for transfection is encapsulated in a lipid nanoparticle (LNP).

Embodiment I-195. The composition of embodiment I-191 or I-194, wherein the shRNA is capable of being expressed and processed in a packaging cell transfected with the polynucleotide into a siRNA sequence complementary to and capable of hybridizing with an mRNA of the CasX transcribed by the packaging cell.

Embodiment I-196. The composition of any one of embodiments I-192 to I-195, wherein the packaging cell is selected from the group consisting of baby hamster kidney (BHK), human embryonic kidney 293 (HEK293), HEK293T, NS0, SP2/0, YO myeloma cells, A549, P3X63 mouse myeloma cells, PER, PER.C6, NIH3T3, COS, HeLa, and Chinese hamster ovary (CHO).

Embodiment I-197. The composition of embodiment I-194 or I-196, wherein upon hybridization of the siRNA sequence to the mRNA of the CasX, the CasX mRNA is degraded such that expression of the CasX protein is reduced or eliminated in the packaging cell.

Embodiment I-198. The composition of embodiment I-197, wherein expression of the CasX protein is reduced by at least 40%, at least 50%, at least 60%, at least 70%, at least 80%, or at least 90% compared to a transfected packaging cell not comprising the shRNA, when assayed in a timed in vitro assay under comparable conditions.

Embodiment I-199. The composition of any one of embodiments I-180 to I-198, wherein the first gRNA comprises a sequence selected from the group of sequences consisting of SEQ ID NOS: 2101-2331 and 3992-3995, or a sequence having at least 85%, at least 90%, at least 95%, at least 95%, at least 96%, at least 97%, at least 98% identity thereto.

Embodiment I-200. The composition of any one of embodiments I-180 to I-198, wherein the first gRNA comprises a sequence selected from the group of sequences consisting of SEQ ID NOS: 2101-2331 and 3992-3995.

Embodiment I-201. The composition of any one of embodiments I-180 to I-198, wherein the first gRNA has a scaffold comprising the sequence of SEQ ID NO: 2296, or a sequence having at least about 70%, at least about 80%, at least about 80%, at least about 85%, at least about 90%, or at least about 95%, or at least about 95%, or at least about 96%, or at least about 97%, or at least about 98%, or at least about 99% sequence identity thereto.

Embodiment I-202. The composition of any one of embodiments I-180 to I-201, wherein the first gRNA comprises a targeting sequence complementary to a target nucleic acid sequence in a cell, wherein the targeting sequence has at least 15, at least 16, at least 17, at least 18, at least 19, at least 20, or at least 21 nucleotides.

Embodiment I-203. The composition of any one of embodiments I-180 to I-202, wherein the AAV expression cassette comprises
a) one or more self-inactivating segments comprising a protospacer adjacent motif (PAM) sequence and a polynucleotide sequence capable of being bound and cleaved by a ribonuclear protein complex (RNP) of the Class 2 CRISPR protein and a second gRNA;
b) a sequence encoding a second gRNA comprising a targeting sequence complementary to the self-inactivating segment; and
c) a third promoter operably linked to the second gRNA.

Embodiment I-204. The composition of embodiment I-203, wherein the one or more self-inactivating segments of the polynucleotide are located:
a) 5′ or 3′ adjacent to or within the sequence encoding the Class 2 CRISPR protein;
b) 5′ or 3′ adjacent to or within a Kozak sequence located between the first promoter and the sequence encoding the Class 2 CRISPR protein;
c) 5′ or 3′ adjacent to or within to the first promoter sequence;
d) 5′ or 3′ adjacent to or within the second promoter sequence;
e) 5′ or 3′ adjacent to or within the third promoter sequence;
f) downstream of the transcriptional start site for the sequence encoding the Class 2 CRISPR protein;
g) within one or more inserted introns in the polynucleotide encoding the Class 2 CRISPR protein;
h) at the 3′ end of the polynucleotide encoding the Class 2 CRISPR protein, between a stop codon and poly(A) termination site of the sequence encoding the Class 2 CRISPR protein; or
i) any combination of (a)-(h).

Embodiment I-205. The composition of embodiment I-203 or I-204, wherein the self-inactivating segment comprises a 15-21 nucleotide sequence complementary to the targeting sequence of the second gRNA that is 3′ adjacent to a PAM sequence recognized by an RNP of the Class 2 CRISPR protein and the second gRNA.

Embodiment I-206. The composition of any one of embodiments I-203 to I-205, wherein cleavage of the self-inactivating segments in a cell transfected with the composition by the RNP of the Class 2 CRISPR protein and the second gRNA results in reduced or eliminated expression of the Class 2 CRISPR protein or the gRNA encoded by the polynucleotide.

Embodiment I-207. The composition of any one of embodiments I-203 to I-206, wherein the PAM sequence of the one or more self-inactivating segments promotes less efficient cleavage or rate of cleavage of the self-inactivating segment by the RNP of the Class 2 CRISPR protein and the second gRNA compared to the PAM sequence 5′ and adjacent to the target nucleic acid of the cell to be modified.

Embodiment I-208. A method for reducing premature cleavage of an self-inactivating AAV (siAAV) transgene encoding a Class 2 CRISPR nuclease protein and one or more gRNAs in a packaging cell, comprising introducing a polynucleotide sequence encoding one or more small hairpin RNA (shRNA) into the packaging cell comprising the siAAV transgene, wherein the shRNA is capable of being expressed and processed into an siRNA sequence, and wherein the siRNA sequence is complementary to an mRNA of the Class 2 CRISPR nuclease transcribed by the packaging cell.

Embodiment I-209. The method of embodiment I-208, wherein the packaging cell is transfected with the siAAV transgene.

Embodiment I-210. The method of embodiment I-208 or I-209, wherein the transgene comprises
a) a first adeno-associated virus (AAV) inverted terminal repeat (ITR) sequence;
b) a second AAV ITR sequence;
c) a sequence encoding a Class 2 CRISPR protein having a single RNA-guided RuvC domain;
d) a first promoter operably linked to the sequence encoding the Class 2 CRISPR protein;
e) a sequence encoding a first guide RNA (gRNA) comprising a targeting sequence that is complementary to and capable of hybridizing with a target nucleic acid of a cell to be modified; and
f) a second promoter sequence operably linked to the sequence encoding the first gRNA
g) a sequence encoding a second guide RNA (gRNA) comprising a scaffold sequence and a linked targeting sequence complementary to one or more self-inactivating segments of the transgene;
h) a third promoter sequence operably linked to the sequence encoding the second gRNA, wherein the third promoter has a sequence different from the sequence of the second promoter; and
i) one or more self-inactivating segments of the polynucleotide comprising a protospacer adjacent motif (PAM) sequence and a polynucleotide sequence capable of being bound and cleaved by a ribonuclear protein complex (RNP) of the Class 2 CRISPR protein and the second gRNA.

Embodiment I-211. The method of any one of embodiments I-208 to I-210, wherein the polynucleotide comprises an encoding sequence for a single shRNA and linked promoter.

Embodiment I-212. The method of any one of embodiments I-208 to I-210, wherein the polynucleotide comprises an encoding sequence for two shRNA and linked promoters.

Embodiment I-213. The method of any one of embodiments I-208 to I-210, wherein the polynucleotide comprises an encoding sequence for three shRNA and linked promoters.

Embodiment I-214. The method of any one of embodiments I-208 to I-213, wherein the shRNA encoding sequence comprises a sequence selected from the group consisting of SEQ ID NOS: 2640-2687, or a sequence having at least 85%, at least 90%, at least 95%, at least 95%, at least 96%, at least 97%, at least 98% identity thereto.

Embodiment I-215. The method of any one of embodiments I-208 to I-213, wherein the shRNA encoding sequence comprises a sequence selected from the group consisting of SEQ ID NOS: 2640-2687.

Embodiment I-216. The method of any one of embodiments I-210 to I-215, wherein the polynucleotide comprising the shRNA and linked promoters are linked exterior to the AAV transgene inserted into a bacterial plasmid backbone.

Embodiment I-217. The method of any one of embodiments I-210 to I-215, wherein the polynucleotide comprising the shRNA and linked promoters are inserted into;
a) an AAV RepCap plasmid;
b) an AAV Helper plasmid; and/or
c) a separate vector.

Embodiment I-218. The method of any one of embodiments I-208 to I-217, wherein the packaging cell is selected from the group consisting of BHK, HEK293, HEK293T, NS0, SP2/0, YO myeloma cells, A549, P3X63 mouse myeloma cells, PER, PER.C6, NIH3T3, COS, HeLa, and CHO.

Embodiment I-219. The method of any one of embodiments I-208 to I-218, wherein upon transcription of the shRNA and Class 2 Type V CRISPR nuclease sequences, the shRNA is processed into siRNA which hybridizes with the mRNA of the Class 2 CRISPR nuclease and is degraded by the packaging cell.

Embodiment I-220. The method of embodiment I-219, wherein expression of the Class 2 CRISPR nuclease protein in the packaging cell is repressed by at least 40%, at least 50%, at least 60%, at least 70%, at least 80%, or at least 90% compared to a transfected packaging cell not comprising the shRNA sequence, when assayed in a timed in vitro assay under comparable conditions.

Embodiment I-221. The method of any one of embodiments I-208 to I-220, wherein the Class 2 CRISPR nuclease protein is a CasX.

Embodiment I-222. The method of embodiment I-221, wherein the encoded CasX comprises a sequence selected from the group consisting of SEQ ID NOS: 1-3, 49-321 and 2356-2488, or a sequence having at least 85%, at least 90%, at least 95%, at least 95%, at least 96%, at least 97%, at least 98%, or at least 99% identity thereto.

Embodiment I-223. The method of embodiment I-221, wherein the encoded CasX comprises a sequence selected from the group consisting of the sequences of SEQ ID NOS: 1-3, 49-321 and 2356-2488.

Embodiment I-224. The method of embodiment I-221, wherein the encoded CasX comprises the sequence of SEQ ID NO: 138, or a sequence having at least about 70%, at least about 80%, at least about 80%, at least about 85%, at least about 90%, or at least about 95%, or at least about 95%, or at least about 96%, or at least about 97%, or at least about 98%, or at least about 99% sequence identity thereto.

Embodiment I-225. The method of any one of embodiments I-208 to I-218, wherein upon transcription of the shRNA and gRNA sequences, shRNA is processed by the packaging cell into siRNA capable of hybridizing with the gRNA.

Embodiment I-226. The method of embodiment I-225, wherein the processed siRNA hybridizes with the gRNA and the gRNA is degraded by the packaging cell.

Embodiment I-227. The method of embodiment I-226, wherein the amount of gRNA in the packaging cell is reduced by at least 40%, at least 50%, at least 60%, at least 70%, at least 80%, or at least 90% compared to a transfected packaging cell not comprising the shRNA sequence, when assayed in a timed in vitro assay under comparable conditions.

Embodiment I-228. The method of any one of embodiments I-210 to I-227, wherein the first gRNA has a scaffold comprising a sequence selected from the group of sequences of SEQ ID NOS: 2101-2331 and 3992-3995, or a sequence having at least about 70%, at least about 80%, at least about 90%, at least about 95%, at least about 96%, at least about 97%, at least about 98%, at least about 99%, or 100% sequence identity thereto.

Embodiment I-229. The method of any one of embodiments I-210 to I--227, wherein the first gRNA has a scaffold comprising a sequence selected from the group of sequences of SEQ ID NOS: 2101-2331 and 3992-3995.

Embodiment I-230. The method of any one of embodiments I-210 to I-229, wherein the second gRNA has a scaffold comprising a sequence selected from the group of sequences of SEQ ID NOS: 2101-2331 and 3992-3995, or a sequence having at least about 70%, at least about 80%, at least about 90%, at least about 95%, at least about 96%, at least about 97%, at least about 98%, at least about 99% sequence identity thereto.

Embodiment I-231. The method of any one of embodiments I-210 to I-229, wherein the second gRNA has a scaffold comprising a sequence selected from the group consisting of sequences SEQ ID NOS: 2101-2331 and 3992-3995.

Embodiment I-232. The method of any one of embodiments I-210 to I-227, wherein the second guide comprises a sequence selected from the group consisting of SEQ ID NO: 2101-2238 and the first guide comprises a sequence selected from the group consisting of SEQ ID NOS: 2276-2296.

Embodiment I-233. The method of embodiment I-232, wherein the second guide comprises the sequence of SEQ ID NO: 2238 or a sequence having at least about 70%, at least about 80%, at least about 90%, at least about 95%, at least about 96%, at least about 97%, at least about 98%, at least about 99% sequence identity thereto and the first guide comprises the sequence of SEQ ID NO: 2296 or a sequence having at least about 70%, at least about 80%, at least about 90%, at least about 95%, at least about 96%, at least about 97%, at least about 98%, at least about 99% sequence identity thereto.

Embodiment I-234. A method for reducing premature cleavage of an siAAV transgene encoding a Class 2 CRISPR nuclease protein and one or more gRNA in a packaging cell, comprising introducing a sequence encoding an interfering RNA (RNAi) into the packaging cell comprising the siAAV transgene, wherein the RNAi is capable of being expressed, and wherein the RNAi sequence is complementary to the gRNA or the mRNA encoding the Class 2 CRISPR nuclease protein transcribed by the packaging cell.

Embodiment I-235. The method of embodiment I-234, wherein the transgene comprises
a) a first adeno-associated virus (AAV) inverted terminal repeat (ITR) sequence;
b) a second AAV ITR sequence;
c) a sequence encoding a Class 2 CRISPR protein having a single RNA-guided RuvC domain;
d) a first promoter operably linked to the sequence encoding the Class 2 CRISPR protein;
e) a sequence encoding a first guide RNA (gRNA) comprising a targeting sequence that is complementary to and capable of hybridizing with a target nucleic acid of a cell to be modified; and
f) a second promoter sequence operably linked to the sequence encoding the first gRNA;
g) a sequence encoding a second guide RNA (gRNA) and a targeting sequence complementary to one or more self-inactivating segments of the transgene;
h) a third promoter sequence operably linked to the sequence encoding the second gRNA, wherein the third promoter has a sequence different from the sequence of the second promoter; and
i) one or more self-inactivating segments of the polynucleotide comprising a protospacer adjacent motif (PAM) sequence and a polynucleotide sequence capable of being bound and cleaved by a ribonuclear protein complex (RNP) of the Class 2 CRISPR protein and the second gRNA.

Embodiment I-236. The method of embodiment I-234 or I-235, wherein the sequence encoding the RNAi is operably linked to a promoter.

Embodiment I-237. The method of embodiment I-236, wherein the sequence encoding the RNAi and linked promoter is linked exterior to the sequence of the siAAV transgene in a bacterial plasmid backbone.

Embodiment I-238. The method of embodiment I-236, wherein the sequence encoding the RNAi and linked promoter is introduced into the packaging cell using a separate vector.

Embodiment I-239. The method of any one of embodiments I-234 to I-238, wherein the cell is a packaging cell selected from the group consisting of BHK, HEK293, HEK293T, NS0, SP2/0, YO myeloma cells, A549, P3X63 mouse myeloma cells, PER, PER.C6, NIH3T3, COS, HeLa, and CHO.

Embodiment I-240. The method of any one of embodiments I-234 to I-239, wherein upon transcription of the gRNA and RNAi, the RNAi hybridizes with the gRNA, interfering with the formation of an RNP of the gRNA and CRISPR nuclease.

Embodiment I-241. The method of any one of embodiments I-234 to I-240, wherein upon transcription of the RNAi, and the mRNA encoding the Class 2 CRISPR nuclease protein, the RNAi hybridizes with the mRNA, interfering with the formation of an RNP of the gRNA and the Class 2 CRISPR nuclease.

Embodiment I-242. The method of embodiment I-240 or I-241, wherein the formation of the RNP in the packaging cell is repressed by at least 40%, at least 50%, at least 60%, at least 70%, at least 80%, or at least 90% compared to a transfected packaging cell not comprising the RNAi sequence, when assayed in a timed in vitro assay under comparable conditions.

Embodiment I-243. The method of any one of embodiments I-240 to I-242, wherein the cleavage of the siAAV transgene in the packaging cell is repressed by at least 40%, at least 50%, at least 60%, at least 70%, at least 80%, or at least 90% compared to a transfected packaging cell not comprising the RNAi sequence, when assayed in a timed in vitro assay under comparable conditions.

Embodiment I-244. The method of any one of embodiments I-234 to I-243, wherein the Class 2 CRISPR nuclease protein is a CasX.

Embodiment I-245. The method of embodiment I-244, wherein the encoded CasX comprises a sequence selected from the group consisting of SEQ ID NOS: 1-3, 49-321 and 2356-2488, or a sequence having at least 85%, at least 90%, at least 95%, at least 95%, at least 96%, at least 97%, at least 98%, or at least 99% identity thereto.

Embodiment I-246. The method of embodiment I-244, wherein the encoded CasX comprises a sequence selected from the group consisting of the sequences of SEQ ID NOS: 1-3, 49-321 and 2356-2488.

Embodiment I-247. The method of embodiment I-244, wherein the encoded CasX comprises the sequence of SEQ ID NO: 138, or a sequence having at least about 70%, at least about 80%, at least about 80%, at least about 85%, at least about 90%, or at least about 95%, or at least about 95%, or at least about 96%, or at least about 97%, or at least about 98%, or at least about 99% sequence identity thereto.

Embodiment I-248. The method of any one of embodiments I-234 to I-247, wherein the first gRNA has a scaffold comprising a sequence selected from the group of sequences of SEQ ID NOS: 2101-2331, 3992-3995 and 4028, or a sequence having at least about 70%, at least about 80%, at least about 90%, at least about 95%, at least about 96%, at least about 97%, at least about 98%, at least about 99%, or 100% sequence identity thereto.

Embodiment I-249. The method of any one of embodiments I-234 to I-247, wherein the first gRNA has a scaffold comprising a sequence selected from the group of sequences of SEQ ID NOS: 2101-2331, 3992-3995 and 4028.

Embodiment I-250. The method of any one of embodiments I-234 to I-249, wherein the second gRNA has a scaffold comprising a sequence selected from the group of sequences of SEQ ID NOS: 2101-2331, 3992-3995, and 4028, or a sequence having at least about 70%, at least about 80%, at least about 90%, at least about 95%, at least about 96%, at least about 97%, at least about 98%, at least about 99%, or 100% sequence identity thereto.

Embodiment I-251. The method of any one of embodiments I-234 to I-249, wherein the second gRNA has a scaffold comprising a sequence selected from the group consisting of sequences SEQ ID NOS: 2101-2331, 3992-3995, and 4028.

Embodiment I-252. The method of any one of embodiments I-234 to I-247, wherein the second guide comprises a sequence selected from the group consisting of SEQ ID NO: 2101-2238 and the first guide comprises a sequence selected from the group consisting of SEQ ID NOS: 2276-2296.

Embodiment I-253. The method of embodiment I-252, wherein the second guide comprises the sequence of SEQ ID NO: 2238 and the first guide comprises the sequence of SEQ ID NO: 2296.

Embodiment I-254. A method for reducing premature cleavage of an siAAV transgene encoding a Class 2 CRISPR nuclease protein and one or more gRNA in a packaging cell, comprising introducing a sequence encoding an anti-sense RNA (asRNA) into the packaging cell comprising the siAAV transgene, wherein the asRNA is capable of being expressed, and wherein the asRNA sequence is complementary to the gRNA or the mRNA encoding the Class 2 CRISPR nuclease protein transcribed by the packaging cell.

Embodiment I-255. The method of embodiment I-254, wherein the transgene comprises
a) a first adeno-associated virus (AAV) inverted terminal repeat (ITR) sequence;
b) a second AAV ITR sequence;
c) a sequence encoding a Class 2 CRISPR protein having a single RNA-guided RuvC domain;
d) a first promoter operably linked to the sequence encoding the Class 2 CRISPR protein;
e) a sequence encoding a first guide RNA (gRNA) comprising a targeting sequence that is complementary to and capable of hybridizing with a target nucleic acid of a cell to be modified; and
f) a second promoter sequence operably linked to the sequence encoding the first gRNA;
g) a sequence encoding a second guide RNA (gRNA) and a targeting sequence complementary to one or more self-inactivating segments of the transgene;
h) a third promoter sequence operably linked to the sequence encoding the second gRNA, wherein the third promoter has a sequence different from the sequence of the second promoter; and
i) one or more self-inactivating segments of the polynucleotide comprising a protospacer adjacent motif (PAM) sequence and a polynucleotide sequence capable of being bound and cleaved by a ribonuclear protein complex (RNP) of the Class 2 CRISPR protein and the second gRNA.

Embodiment I-256. The method of embodiment I-254 or I-255, wherein the sequence encoding the asRNA is operably linked to a promoter.

Embodiment I-257. The method of embodiment I-256, wherein the sequence encoding the asRNA and linked promoter is linked to the 5′ end of the siAAV transgene.

Embodiment I-258. The method of embodiment I-256, wherein the sequence encoding the asRNA and linked promoter is introduced into the packaging cell using a separate vector.

Embodiment I-259. The method of any one of embodiments I-254 to I-258, wherein the cell is a packaging cell selected from the group consisting of BHK, HEK293, HEK293T, NS0, SP2/0, YO myeloma cells, A549, P3X63 mouse myeloma cells, PER, PER.C6, NIH3T3, COS, HeLa, and CHO.

Embodiment I-260. The method of any one of embodiments I-254 to I-259, wherein upon transcription of the gRNA and asRNA in the packaging cell, the asRNA hybridizes with the gRNA, interfering with the formation of an RNP of the gRNA and CRISPR nuclease.

Embodiment I-261. The method of any one of embodiments I-254 to I-259, wherein upon transcription of the mRNA of the Class 2 CRISPR nuclease protein and the asRNA, the asRNA hybridizes with the mRNA, repressing expression of the Class 2 CRISPR nuclease protein in the packaging cell.

Embodiment I-262. The method of embodiment I-260 or I-261, wherein the formation of the RNP in the packaging cell is repressed by at least 40%, at least 50%, at least 60%, at least 70%, at least 80%, or at least 90% compared to a transfected packaging cell not comprising the asRNA sequence, when assayed in a timed in vitro assay under comparable conditions.

Embodiment I-263. The method of embodiment I-260 or I-261, wherein the cleavage of the siAAV transgene in the packaging cell is repressed by at least 40%, at least 50%, at least 60%, at least 70%, at least 80%, or at least 90% compared to a transfected packaging cell not comprising the asRNA sequence, when assayed in a timed in vitro assay under comparable conditions.

Embodiment I-264. The method of embodiment I-260 or I-261, wherein the titer of the siAAV produced by the packaging cell comprising the asRNA is at least about 2-fold, at least about 3-fold, at least about 4-fold, at least about 5-fold, at least about 6-fold, at least about 7-fold, at least about 8-fold, at least about 9-fold, at least about 10-fold higher compared to the titer produced using a comparable siAAV construct not comprising the asRNA.

Embodiment I-265. The method of any one of embodiments I-254 to I-264, wherein the Class 2 CRISPR nuclease protein is a CasX.

Embodiment I-266. The method of embodiment I-265, wherein the encoded CasX comprises a sequence selected from the group consisting of SEQ ID NOS: 1-3, 49-321 and 2356-2488, or a sequence having at least 85%, at least 90%, at least 95%, at least 95%, at least 96%, at least 97%, at least 98%, or at least 99% identity thereto.

Embodiment I-267. The method of embodiment I-265, wherein the encoded CasX comprises a sequence selected from the group consisting of the sequences of SEQ ID NOS: 1-3, 49-321 and 2356-2488.

Embodiment I-268. The method of embodiment I-267, wherein the encoded CasX comprises the sequence of SEQ ID NO: 138, or a sequence having at least about 70%, at least about 80%, at least about 80%, at least about 85%, at least about 90%, or at least about 95%, or at least about 95%, or at least about 96%, or at least about 97%, or at least about 98%, or at least about 99% sequence identity thereto.

Embodiment I-269. The method of any one of embodiments I-254 to I-267, wherein the gRNA has a scaffold comprising a sequence selected from the group of sequences consisting of the sequences of SEQ ID NOS: 2101-2331, 3992-3995, and 4028, or a sequence having at least at least about 70%, at least about 80%, at least about 80%, at least about 85%, at least about 90%, or at least about 95%, or at least about 95%, or at least about 96%, or at least about 97%, or at least about 98%, or at least about 99% sequence identity thereto.

Embodiment I-270. The method of any one of embodiments I-254 to I-267, wherein the gRNA has a scaffold comprising a sequence selected from the group consisting of the sequences of SEQ ID NOS: 2101-2331, 3992-3995, and 4028.

Embodiment I-271. The method of embodiment I-270, wherein the gRNA has a scaffold comprising the sequence of SEQ ID NO: 2296, or a sequence having at least about 70%, at least about 80%, at least about 80%, at least about 85%, at least about 90%, or at least about 95%, or at least about 95%, or at least about 96%, or at least about 97%, or at least about 98%, or at least about 99% sequence identity thereto.

Embodiment I-272. A method for reducing premature cleavage of an siAAV transgene in a transfected packaging cell, wherein the siAAV transgene comprises one or more self-inactivating sequences and encodes a Class 2 CRISPR nuclease protein, a first gRNA comprising a targeting sequence that is complementary to and capable of hybridizing with a target nucleic acid of a cell to be modified, a second gRNA comprising a targeting sequence that is complementary to and capable of hybridizing with the one or more self-inactivating sequences, wherein the method comprises introducing a sequence encoding a third, non-targeting gRNA into the packaging cell transfected with the transgene, wherein the CRISPR nuclease protein and the first, the second, and the third gRNA are each capable of being expressed and each are capable of binding to the CRISPR nuclease protein.

Embodiment I-273. The method of embodiment I-272, wherein the sequence encoding the third gRNA is operably linked to a promoter of equal or stronger strength compared to a promoter operably linked to the first and the second gRNA.

Embodiment I-274. The method of embodiment I-273, wherein the promoters are selected from the group consisting of U6, mini U6, 5S, Adenovirus 2 (Ad2) VAI, 7SK, H1, bidirectional H1, bidirectional U6, bidirectional 7SK, and bidirectional U6.

Embodiment I-275. The method of embodiment I-274, wherein the third promoter is U6 and the first and second promoters are selected from the group consisting of H1, 7SK, and mini U6.

Embodiment I-276. The method of any one of embodiments I-272 to I-275, wherein the sequence encoding the third gRNA and linked promoter is linked to the 5′ end of the siAAV transgene.

Embodiment I-277. The method of any one of embodiments I-272 to I-275, wherein the sequence encoding the third gRNA and linked promoter is introduced into the packaging cell using a separate vector.

Embodiment I-278. The method of any one of embodiments I-272 to I-277, wherein the cell is a packaging cell selected from the group consisting of BHK, HEK293, HEK293T, NS0, SP2/0, YO myeloma cells, A549, P3X63 mouse myeloma cells, PER, PER.C6, NIH3T3, COS, HeLa, and CHO.

Embodiment I-279. The method of any one of embodiments I-272 to I-278, wherein upon expression of the third gRNA and CRISPR nuclease protein in the packaging cell, the third gRNA competes with the second gRNA for complexing with the CRISPR nuclease protein as an RNP, reducing the ability of the CRISPR nuclease to cleave the siAAV transgene.

Embodiment I-280. The method of embodiment I-279, wherein the cleavage of the siAAV transgene in the packaging cell is reduced by at least 40%, at least 50%, at least 60%, at least 70%, at least 80%, or at least 90% compared to a transfected packaging cell not comprising the third gRNA sequence, when assayed in a timed in vitro assay under comparable conditions.

Embodiment I-281. The method of embodiment I-279, wherein the titer of the siAAV produced by the packaging cell comprising the non-targeting gRNA is at least about 2-fold, at least about 3-fold, at least about 4-fold, at least about 5-fold, at least about 6-fold, at least about 7-fold, at least about 8-fold, at least about 9-fold, at least about 10-fold higher compared to the titer produced using a comparable siAAV construct not comprising the non-targeting gRNA.

Embodiment I-282. The method of any one of embodiments I-272 to I-281, wherein the Class 2 CRISPR nuclease protein is a CasX.

Embodiment I-283. The method of embodiment I-282, wherein the encoded CasX comprises a sequence selected from the group consisting of SEQ ID NOS: 1-3, 49-321 and 2356-2488, or a sequence having at least 85%, at least 90%, at least 95%, at least 95%, at least 96%, at least 97%, at least 98%, or at least 99% identity thereto.

Embodiment I-284. The method of embodiment I-282, wherein the encoded CasX comprises a sequence selected from the group consisting of the sequences of SEQ ID NOS: 1-3, 49-321 and 2356-2488.

Embodiment I-285. The method of embodiment I-282, wherein the Class 2 CRISPR protein is a CasX protein comprising the sequence of SEQ ID NO: 138, or a sequence having at least about 70%, at least about 80%, at least about 80%, at least about 85%, at least about 90%, or at least about 95%, or at least about 95%, or at least about 96%, or at least about 97%, or at least about 98%, or at least about 99% sequence identity thereto.

Embodiment I-286. The method of any one of embodiments I-272 to I-285, wherein the gRNA each have a scaffold comprising a sequence selected from the group of sequences consisting of the sequences of SEQ ID NOS: 2101-2331, 3992-3995, and 4028, or a sequence having at least at least about 70%, at least about 80%, at least about 80%, at least about 85%, at least about 90%, or at least about 95%, or at least about 95%, or at least about 96%, or at least about 97%, or at least about 98%, or at least about 99% sequence identity thereto.

Embodiment I-287. The method of any one of embodiments I-272 to I-285, wherein the gRNA each have a scaffold comprising a sequence selected from the group consisting of the sequences of SEQ ID NOS: 2101-2331, 3992-3995, 4028.

Embodiment I-288. The method of embodiment I-287, wherein the gRNA each have a scaffold comprising a sequence of SEQ ID NO: 2296.

### Set II

Embodiment II-1. A self-inactivating recombinant vector (SIRV) comprising a polynucleotide comprising:
a) one or more packaging components;
b) a sequence encoding a Class 2 Type V protein comprising a single RNA-guided RuvC domain;
c) a first promoter operably linked to the sequence encoding the Class 2 Type V protein;
d) a sequence encoding a first guide RNA (gRNA) comprising a scaffold sequence linked to a targeting sequence that is complementary to and capable of hybridizing with: 1) a target nucleic acid of a cell to be modified; and 2) one or more self-inactivating segments incorporated in the polynucleotide;
e) a second promoter sequence operably linked to the sequence encoding the first gRNA; and
f) one or more self-inactivating segments comprising a protospacer adjacent motif (PAM) sequence and a polynucleotide sequence capable of being bound and cleaved by a ribonuclear protein complex (RNP) comprising the Class 2 Type V protein and the first gRNA.

Embodiment II-2. The SIRV of embodiment II-1, wherein the one or more self-inactivating segments of the polynucleotide are located:
a) 5′ or 3′ adjacent to or within the sequence encoding the Class 2 Type V protein;
b) 5′ or 3′ adjacent to or within a Kozak sequence located between the first promoter and the sequence encoding the Class 2 Type V protein;
c) 5′ or 3′ adjacent to or within to the first promoter sequence;
d) 5′ or 3′ adjacent to or within the second promoter sequence;
e) 3′ downstream of the transcriptional start site for the sequence encoding the Class 2 Type V protein;
f) within one or more inserted introns in the polynucleotide encoding the Class 2 Type V protein;
g) at the 3′ end of the polynucleotide encoding the Class 2 Type V protein, between a stop codon and poly(A) termination site for the Class2 Type V protein; or
h) any combination of (a)-(g).

Embodiment II-3. The SIRV of any one of embodiments II-1 to II-2, wherein the self-inactivating segment comprises a sequence corresponding to any 15-21 nucleotide portion of the target nucleic acid sequence that is 3′ adjacent to a PAM sequence recognized by an RNP of the Class 2 Type V protein and the first gRNA.

Embodiment II-4. The SIRV of any one of embodiments II-1 to II-3, wherein the PAM sequence of the one or more self-inactivating segments:
a) is different by at least one nucleotide from the PAM sequence of the target nucleic acid of the cell to be modified; and
b) promotes less efficient cleavage or rate of cleavage of the self-inactivating segment by the RNP compared to the target nucleic acid of the cell to be modified.

Embodiment II-5. The SIRV of embodiment II-4, wherein:
a) if the PAM sequence of the target nucleic acid of the cell to be modified is TTC and the PAM preference of the Class 2 Type V protein is TTC, then the PAM sequence of the one or more self-inactivating segments is selected from the group consisting of ATC, CTC, TTT, GTT, and GTC;
b) if the PAM sequence of the target nucleic acid of the cell to be modified is ATC and the PAM preference of the Class 2 Type V protein is TTC, then the PAM sequence of the one or more self-inactivating segments is selected from the group consisting of CTC, TTT, GTT, and GTC; or
c) if the PAM sequence of the target nucleic acid of the cell to be modified is CTC and the PAM preference of the Class 2 Type V protein is TTC, then the PAM sequence of the one or more self-inactivating segments is TTT, GTT, ATC, or GTC.

Embodiment II-6. The SIRV of embodiment II-4, wherein:
a) if the PAM sequence of the target nucleic acid of the cell to be modified is ATC and the PAM preference of the Class 2 Type V protein is ATC, then the PAM sequence of the one or more self-inactivating segments is selected from the group consisting of TTC, CTC, TTT, GTT, and GTC;
b) if the PAM sequence of the target nucleic acid of the cell to be modified is CTC and the PAM preference of the Class 2 Type V protein is ATC, then the PAM sequence of the one or more self-inactivating segments is selected from the group consisting of TTC, TTT, GTT, and GTC; or
c) if the PAM sequence of the target nucleic acid of the cell to be modified is GTC and the PAM preference of the Class 2 Type V protein is ATC, then the PAM sequence of the one or more self-inactivating segments is TTC, TTT, CTC, or GTT.

Embodiment II-7. The SIRV of embodiment II-4, wherein:
a) if the PAM sequence of the target nucleic acid of the cell to be modified is GTC and the PAM preference of the Class 2 Type V protein is GTC, then the PAM sequence of the one or more self-inactivating segments is selected from the group consisting of ATC, CTC, TTT, GTT, and TTC.
b) if the PAM sequence of the target nucleic acid of the cell to be modified is GTC and the PAM preference of the Class 2 Type V protein is ATC, then the PAM sequence of the one or more self-inactivating segments is selected from the group consisting of TTC, TTT, GTT, and CTC; or
c) if the PAM sequence of the target nucleic acid of the cell to be modified is CTC and the PAM preference of the Class 2 Type V protein is GTC, then the PAM sequence of the one or more self-inactivating segments is TTC, TTT, ATC, or GTT.

Embodiment II-8. The SIRV of embodiment II-4, wherein:
a) if the PAM sequence of the target nucleic acid of the cell to be modified is CTC and the PAM preference of the Class 2 Type V protein is CTC, then the PAM sequence of the one or more self-inactivating segments is selected from the group consisting of ATC, GTC, TTT, GTT, and TTC.
b) if the PAM sequence of the target nucleic acid of the cell to be modified is ATC and the PAM preference of the Class 2 Type V protein is CTC, then the PAM sequence of the one or more self-inactivating segments is selected from the group consisting of GTC, TTT, GTT, and TTC; or
c) if the PAM sequence of the target nucleic acid of the cell to be modified is GTC and the PAM preference of the Class 2 Type V protein is CTC, then the PAM sequence of the one or more self-inactivating segments is TTC, TTT, ATC, or GTT.

Embodiment II-9. The SIRV of any one of embodiments II-1 to II-8, wherein the one or more self-inactivating segments each have between about 1 to about 5 bases that are not individually complementary to corresponding positions in the targeting sequence of the first gRNA.

Embodiment II-10. The SIRV of any one of embodiments II-1 to II-9, wherein the percent cleavage by the RNP of the self-inactivating segments of the polynucleotide in a cell transfected or transduced with the SIRV is at least about 10%, at least about 20%, at least about 30%, at least about 40%, at least about 50%, at least about 60%, at least about 70%, at least about 80%, or at least about 90% less than the cleavage of the target nucleic acid in the cell in a timed in vitro cell-based assay, when assayed under comparable conditions.

Embodiment II-11. The SIRV of any one of embodiments II-1 to II-9, wherein the time to achieve 90% cleavage by the RNP of the self-inactivating segments of the polynucleotide in a cell transfected or transduced with the SIRV is delayed, relative to the time to achieve 90% editing of the target nucleic acid in the cell, by at least about 1 day, at least about 2 days, at least about 3 days, at least about 4 days, at least about 5 days, at least about 6 days, at least about 7 days, at least about 8 days, or at least about 9 days, when assayed in an in vitro assay under comparable conditions.

Embodiment II-12. The SIRV of any one of embodiments II-1 to II-11, wherein cleavage by the RNP of the self-inactivating segments of the polynucleotide in a cell transfected or transduced with the SIRV has a k_{cleave} rate that is at least about 2-fold, at least about 4-fold, at least about 5-fold, at least about 6-fold, at least about 7-fold, at least about 8-fold, at least about 9-fold, or at least about 10-fold less than the k_{cleave} rate of the target nucleic acid in an in vitro cell-based assay, when assayed under comparable conditions.

Embodiment II-13. The SIRV of any one of embodiments II-1 to II-12, wherein cleavage by the RNP of the self-inactivating segment of the polynucleotide in a cell transduced or transfected with the SIRV results in reduced or eliminated expression of the Class 2 Type V protein or the gRNA encoded by the polynucleotide.

Embodiment II-14. The SIRV of any one of embodiments II-1 to II-13, wherein the Class 2 Type V protein further comprises one or more nuclear localization signals (NLS) located at or near the N-terminus and/or at or near the C-terminus of the Class 2 Type V protein.

Embodiment II-15. The SIRV of any one of embodiments II-1 to II-14, wherein the one or more NLS are selected from the group of sequences consisting of PKKKRKV (SEQ ID NO: 344), KRPAATKKAGQAKKKK (SEQ ID NO: 345), PAAKRVKLD (SEQ ID NO: 346), RQRRNELKRSP (SEQ ID NO: 347), NQSSNFGPMKGGNFGGRSSGPYGGGGQYFAKPRNQGGY (SEQ ID NO: 348), RMRIZFKNKGKDTAELRRRRVEVSVELRKAKKDEQILKRRNV (SEQ ID NO: 349), VSRKRPRP (SEQ ID NO: 350), PPKKARED (SEQ ID NO: 351), PQPKKKPL (SEQ ID NO:352), SALIKKKKKMAP (SEQ ID NO: 353), DRLRR (SEQ ID NO: 354), PKQKKRK (SEQ ID NO: 355), RKLKKKIKKL (SEQ ID NO: 356), REKKKFLKRR (SEQ ID NO: 357), KRKGDEVDGVDEVAKKKSKK (SEQ ID NO: 358), RKCLQAGMNLEARKTKK (SEQ ID NO: 359), PRPRKIPR (SEQ ID NO: 360), PPRKKRTVV (SEQ ID NO: 361), NLSKKKKRKREK (SEQ ID NO: 362), RRPSRPFRKP (SEQ ID NO: 363), KRPRSPSS (SEQ ID NO: 364), KRGINDRNFWRGENERKTR (SEQ ID NO: 365), PRPPKMARYDN (SEQ ID NO: 366), KRSFSKAF (SEQ ID NO: 367), KLKIKRPVK (SEQ ID NO: 368), PKTRRRPRRSQRKRPPT (SEQ ID NO: 370), SRRRKANPTKLSENAKKLAKEVEN (SEQ ID NO: 371), KTRRRPRRSQRKRPPT (SEQ ID NO: 372), RRKKRRPRRKKRR (SEQ ID NO: 373), PKKKSRKPKKKSRK (SEQ ID NO: 374), HKKKHPDASVNFSEFSK (SEQ ID NO: 375), QRPGPYDRPQRPGPYDRP (SEQ ID NO: 376), LSPSLSPLLSPSLSPL (SEQ ID NO: 377), RGKGGKGLGKGGAKRHRK (SEQ ID NO: 378), PKRGRGRPKRGRGR (SEQ ID NO: 379), MSRRRKANPTKLSENAKKLAKEVEN (SEQ ID NO: 598), PKKKRKVPPPPAAKRVKLD (SEQ ID NO: 380), PKKKRKVPPPPKKKRKV (SEQ ID NO: 381), PAKRARRGYKC (SEQ ID NO: 382); KLGPRKATGRW (SEQ ID NO: 383), PRRKREE (SEQ ID NO: 384), PLRKRPRR (SEQ ID NO: 386), PLRKRPRRGSPLRKRPRR (SEQ ID NO: 387), PAAKRVKLDGGKRTADGSEFESPKKKRKV (SEQ ID NO: 388), PAAKRVKLDGGKRTADGSEFESPKKKRKVGIHGVPAA (SEQ ID NO: 389), PAAKRVKLDGGKRTADGSEFESPKKKRKVAEAAAKEAAAKEAAAKA (SEQ ID NO: 390), PAAKRVKLDGGKRTADGSEFESPKKKRKVPG (SEQ ID NO: 391), KRKGSPERGERKRHW (SEQ ID NO: 392), KRTADSQHSTPPKTKRKVEFEPKKKRKV (SEQ ID NO: 393), PKKKRKVGGSKRTADSQHSTPPKTKRKVEFEPKKKRKV (SEQ ID NO: 394), MAPKKKRKVSR (SEQ ID NO: 771), and MAPKKKRKVGGSPKKKRKVGGSPKKKRKVGGSPKKKRKVSR (SEQ ID NO: 772) wherein the one or more NLS are linked to the Type V protein or to adjacent NLS with a linker peptide wherein the linker peptide is selected from the group consisting of RS, (G)n (SEQ ID NO: 395), (GS)n (SEQ ID NO: 396), (GSGGS)n (SEQ ID NO: 397), (GGSGGS)n (SEQ ID NO: 398), (GGGS)n (SEQ ID NO: 399), GGSG (SEQ ID NO: 400), GGSGG (SEQ ID NO: 401), GSGSG (SEQ ID NO: 402), GSGGG (SEQ ID NO: 403), GGGSG (SEQ ID NO: 404), GSSSG (SEQ ID NO:405), GPGP (SEQ ID NO: 406), GGP, PPP, PPAPPA (SEQ ID NO: 407), PPPG (SEQ ID NO: 408), PPPGPPP (SEQ ID NO: 409), PPP(GGGS)n (SEQ ID NO: 410), (GGGS)nPPP (SEQ ID NO: 411), AEAAAKEAAAKEAAAKA (SEQ ID NO: 412), and TPPKTKRKVEFE (SEQ ID NO: 413), wherein n is 1 to 5.

Embodiment II-16. The SIRV of any one of embodiments II-1 to II-14, wherein the one or more encoded NLS are selected from the group consisting of SEQ ID NOS: 538-597, 599-610, 613, 771-772, 844-846, and 2498-2591 set forth in Table 7, Table 22 and Table 23.

Embodiment II-17. The SIRV of any one of embodiments II-1 to II-16, wherein the Class 2 Type V protein is a CasX protein selected from the group of sequences consisting of SEQ ID NOs: 1-3, 49-321 and 2356-2488, or a sequence having at least about 70%, at least about 80%, at least about 80%, at least about 85%, at least about 90%, or at least about 95%, or at least about 95%, or at least about 96%, or at least about 97%, or at least about 98%, or at least about 99% sequence identity thereto.

Embodiment II-18. The SIRV of any one of embodiments II-1 to II-17, wherein the first gRNA has a scaffold comprising a sequence selected from the group of sequences consisting of SEQ ID NOS: 2101-2331, 3992-3995, and 4028 or a sequence having at least about 70%, at least about 80%, at least about 80%, at least about 85%, at least about 90%, or at least about 95%, or at least about 95%, or at least about 96%, or at least about 97%, or at least about 98%, or at least about 99% sequence identity thereto.

Embodiment II-19. The SIRV of any one of embodiments II-1 to II-19, wherein the first gRNA comprises a targeting sequence having 15 nucleotides, 16 nucleotides, 17, nucleotides, 18 nucleotides, 19 nucleotides, or 20 nucleotides.

Embodiment II-20. The SIRV of any one of embodiments II-17 to II-19, wherein the CasX protein is capable of forming a ribonuclear protein complex (RNP) with the first gRNA upon expression in the cell.

Embodiment II-21. The SIRV of embodiment II-20, wherein the RNP is capable of cleaving the target nucleic acid and the self-inactivating segment.

Embodiment II-22. The SIRV of any one of embodiments II-1 to II-21, wherein the packaging element comprises AAV 5′ and 3′ inverted terminal repeats (ITR), wherein the AAV 5′ and 3′ ITRs are derived from serotype AAV1, AAV2, AAV3, AAV4, AAV5, AAV6, AAV7, AAV8, AAV9, AAV10, AAV11, AAV12, AAV 44.9, AAV 9.45, AAV 9.61, AAV-Rh74, AAVRh10, or chimeric combinations thereof.

Embodiment II-23. The SIRV of embodiment II-22, wherein the ITRs are derived from serotype AAV2.

Embodiment II-24. An SIRV comprising a polynucleotide comprising:
a) one or more packaging components;
b) a sequence encoding a Class 2 Type V protein;
c) a first promoter operably linked to the sequence encoding the Class 2 Type V protein;
d) a sequence encoding a first guide RNA (gRNA) comprising a scaffold sequence and a linked targeting sequence that is complementary to a target nucleic acid of a cell to be modified;
e) a second promoter sequence operably linked to the sequence encoding the first gRNA; and one or more of:
f) a sequence encoding a second gRNA comprising a targeting sequence complementary to both a target nucleic acid of a cell to be modified and to one or more self-inactivating segments of the SIRV, wherein the second gRNA comprises a scaffold sequence identical to the scaffold sequence of the first gRNA, wherein:
   1) the sequence of the one or more self-inactivating segments is different by one or more nucleotides from the sequence of the target nucleic acid of the cell to be modified and promotes less efficient cleavage or rate of cleavage of the self-inactivating segment by the RNP compared to the target nucleic acid of the cell to be modified; and
   2) the targeting sequence of the second gRNA is complementary to different or overlapping regions of the target nucleic acid sequence compared to the targeting sequence of the first gRNA;
g) a sequence encoding a second gRNA comprising a targeting sequence complementary to the one or more self-inactivating segments, the second gRNA comprising a scaffold sequence different from the scaffold sequence of the first gRNA, wherein the second gRNA promotes less efficient editing and/or cleavage by an RNP comprising the Class 2 Type V protein and the second gRNA compared to an RNP comprising the Class 2 Type V protein and the first gRNA;
h) a sequence encoding a second gRNA comprising a targeting sequence complementary to both a target nucleic acid of a cell to be modified and to one or more self-inactivating segments of the SIRV, wherein the second gRNA comprises a scaffold sequence identical to the scaffold sequence of the first gRNA, wherein:
   1) the PAM sequence of the one or more self-inactivating segments is different by at least one nucleotide from the PAM sequence of the target nucleic acid of the cell to be modified and promotes less efficient cleavage or rate of cleavage of the self-inactivating segment by the RNP compared to the target nucleic acid of the cell to be modified; and
   2) the targeting sequence of the second gRNA is complementary to different or overlapping regions of the target nucleic acid sequence compared to the targeting sequence of the first gRNA; and
i) a third promoter sequence operably linked to the sequence encoding the second gRNA;
wherein the polynucleotide comprises one or more self-inactivating segments comprising a protospacer adjacent motif (PAM) sequence and a polynucleotide sequence capable of being bound and cleaved by a ribonuclear protein complex (RNP) of the Class 2 Type V protein and the second gRNA.

Embodiment II-25. The SIRV of embodiment II-24, comprising components (a)-(f), and (i).

Embodiment II-26. The SIRV of embodiment II-24, comprising components (a)-(e), (g), and (i).

Embodiment II-27. The SIRV of embodiment II-24, comprising components (a)-(e), (h) and (i).

Embodiment II-28. The SIRV of any one of embodiments II-24 to II-27, wherein the one or more self-inactivating segments of the polynucleotide are located:
a) 5′ or 3′ adjacent to or within the sequence encoding the Class 2 Type V protein;
b) 5′ or 3′ adjacent to or within a Kozak sequence located between the first promoter and the sequence encoding the Class 2 Type V protein;
c) 5′ or 3′ adjacent to or within to the first promoter sequence;
d) 5′ or 3′ adjacent to or within the second promoter sequence;
e) 5′ or 3′ adjacent to or within the third promoter sequence;
f) 3′ downstream of the transcriptional start site for the sequence encoding the Class 2 Type V protein;
g) within one or more inserted introns in the sequence encoding the Class 2 Type V protein;
h) at the 3′ end of the polynucleotide encoding the Class 2 Type V protein, between a stop codon and poly(A) termination site of the sequence encoding the Class 2 Type V; or
i) any combination of (a)-(h).

Embodiment II-29. The SIRV of embodiment II-28, wherein the self-inactivating segment comprises a 15-21 nucleotide sequence complementary to the targeting sequence of the second gRNA and that is 3′ adjacent to a PAM sequence recognized by an RNP of the Class 2 Type V protein and the second gRNA.

Embodiment II-30. The SIRV of any one of embodiments II-24 to II-29, wherein cleavage of the self-inactivating segments in a cell transduced or transfected with the SIRV by the RNP of the Class 2 Type V protein and the second gRNA results in reduced or eliminated expression of the Class 2 Type V protein or the gRNA encoded by the polynucleotide.

Embodiment II-31. The SIRV of any one of embodiments II-24 to II-30, wherein the PAM sequence of the one or more self-inactivating segments:
a) is different from the PAM sequence of the target nucleic acid of the cell to be modified; and
b) promotes less efficient cleavage or rate of cleavage of the self-inactivating segment by the RNP of the Class 2 Type V protein and the second gRNA compared to the PAM of the target nucleic acid of the cell to be modified.

Embodiment II-32. The SIRV of embodiment II-31, wherein:
a) if the PAM sequence of the target nucleic acid of the cell to be modified is TTC and the PAM preference of the Class 2 Type V protein is TTC, then the PAM sequence of the one or more self-inactivating segments is selected from the group consisting of ATC, CTC, TTT, GTT, and GTC;
b) if the PAM sequence of the target nucleic acid of the cell to be modified is ATC and the PAM preference of the Class 2 Type V protein is TTC, then the PAM sequence of the one or more self-inactivating segments is selected from the group consisting of CTC, TTT, GTT, and GTC; or
c) if the PAM sequence of the target nucleic acid of the cell to be modified is CTC and the PAM preference of the Class 2 Type V protein is TTC, then the PAM sequence of the one or more self-inactivating segments is GTC, TTT, ATC, or GTT.

Embodiment II-33. The SIRV of embodiment II-31, wherein:
a) if the PAM sequence of the target nucleic acid of the cell to be modified is ATC and the PAM preference of the Class 2 Type V protein is ATC, then the PAM sequence of the one or more self-inactivating segments is selected from the group consisting of TTC, CTC, TTT, GTT, and GTC;
b) if the PAM sequence of the target nucleic acid of the cell to be modified is CTC and the PAM preference of the Class 2 Type V protein is ATC, then the PAM sequence of the one or more self-inactivating segments is selected from the group consisting of TTC, TTT, GTT, and GTC; or
c) if the PAM sequence of the target nucleic acid of the cell to be modified is GTC and the PAM preference of the Class 2 Type V protein is ATC, then the PAM sequence of the one or more self-inactivating segments is TTC, TTT, CTC, or GTT.

Embodiment II-34. The SIRV of embodiment II-31, wherein:
a) if the PAM sequence of the target nucleic acid of the cell to be modified is GTC and the PAM preference of the Class 2 Type V protein is GTC, then the PAM sequence of the one or more self-inactivating segments is selected from the group consisting of ATC, CTC, TTT, GTT, and TTC.
b) if the PAM sequence of the target nucleic acid of the cell to be modified is GTC and the PAM preference of the Class 2 Type V protein is ATC, then the PAM sequence of the one or more self-inactivating segments is selected from the group consisting of TTC, TTT, GTT, and CTC; or
c) if the PAM sequence of the target nucleic acid of the cell to be modified is CTC and the PAM preference of the Class 2 Type V protein is GTC, then the PAM sequence of the one or more self-inactivating segments is TTC, TTT, ATC, or GTT.

Embodiment II-35. The SIRV of embodiment II-31, wherein:
a) if the PAM sequence of the target nucleic acid of the cell to be modified is CTC and the PAM preference of the Class 2 Type V protein is CTC, then the PAM sequence of the one or more self-inactivating segments is selected from the group consisting of ATC, GTC, TTT, GTT, and TTC.
b) if the PAM sequence of the target nucleic acid of the cell to be modified is ATC and the PAM preference of the Class 2 Type V protein is CTC, then the PAM sequence of the one or more self-inactivating segments is selected from the group consisting of GTC, TTT, GTT, and TTC; or
c) if the PAM sequence of the target nucleic acid of the cell to be modified is GTC and the PAM preference of the Class 2 Type V protein is CTC, then the PAM sequence of the one or more self-inactivating segments is TTC, TTT, ATC, or GTT.

Embodiment II-36. The SIRV of any one of embodiments II-24 to II-35, wherein the one or more self-inactivating segment sequences each have between 1 to 5 bases that are not complementary to corresponding positions in the targeting sequence of the second gRNA.

Embodiment II-37. The SIRV of any of embodiments II-24 to II-36, wherein the RNP of the Class 2 Type V protein and second gRNA exhibit less efficient cleavage of the self-inactivating segment compared to the cleavage of the target nucleic acid of the cell by the RNP of the Class 2 Type V protein and first gRNA.

Embodiment II-38. The SIRV of any one of embodiments II-24 to II-37, wherein the third promoter sequence is different from the second promoter sequence and is less efficient at initiating transcription of the second gRNA compared to the second promoter initiating transcription of the first gRNA.

Embodiment II-39. The SIRV of embodiment II-38, wherein the second promoter is U6 and the third promoter is selected from the group consisting of H1, 7SK, and mini U6.

Embodiment II-40. The SIRV of any one of embodiments II-24 to II-39, wherein the Class 2 Type V protein further comprises one or more nuclear localization signals (NLS).

Embodiment II-41. The SIRV of any one of embodiments II-24 to II-40, wherein the Type V protein is a CasX protein selected from the group consisting of SEQ ID NOs: 1-3 and 49-321 and 2356-2488, or a sequence having at least about 70%, at least about 80%, at least about 80%, at least about 85%, at least about 90%, or at least about 95%, or at least about 95%, or at least about 96%, or at least about 97%, or at least about 98%, or at least about 99% sequence identity thereto.

Embodiment II-42. The SIRV of any one of embodiments II-24 to II-41, wherein the second guide comprises a sequence selected from the group consisting of SEQ ID NO: 2101-2238 and the first guide comprises a sequence selected from the group consisting of SEQ ID NOS: 2276-2296.

Embodiment II-43. The SIRV of any one of embodiments II-24 to II-42, wherein the first and second gRNA each comprise a targeting sequence having 15 nucleotides, 16 nucleotides, 17, nucleotides, 18 nucleotides, 19 nucleotides, or 20 nucleotides.

Embodiment II-44. The SIRV of any one of embodiments II-41 to II-43, wherein the CasX protein is capable of forming a ribonuclear protein complex (RNP) with the first gRNA and the second gRNA upon expression in a cell transduced or transfected with the SIRV.

Embodiment II-45. The SIRV of embodiment II-44, wherein the RNP of the CasX protein and the first gRNA is capable of cleaving the target nucleic acid and wherein the RNP of the CasX protein and the second gRNA is capable of cleaving the self-inactivating segment, and wherein the RNP of the CasX protein and the second gRNA exhibit a cleavage rate of the self-inactivating segments that is less efficient compared to the cleavage or rate of cleavage of the target nucleic acid by an RNP of the CasX protein and the first gRNA.

Embodiment II-46. The SIRV of any one of embodiments II-24 to II-45, wherein the packaging element is an AAV 5′ and 3′ inverted terminal repeat (ITR), wherein the AAV 5′ and 3′ ITRs are derived from serotype AAV1, AAV2, AAV3, AAV4, AAV5, AAV6, AAV7, AAV8, AAV9, AAV10, AAV11, AAV12, AAV 44.9, AAV 9.45, AAV 9.61, AAV-Rh74, AAVRh10, or a chimeric combination thereof.

Embodiment II-47. The SIRV of any one of embodiments II-1 to II-46, wherein the polynucleotide comprises a sequence selected from the group consisting of SEQ ID NOs 4151-4156, or a sequence having at least about 70%, at least about 80%, at least about 80%, at least about 85%, at least about 90%, or at least about 95%, or at least about 95%, or at least about 96%, or at least about 97%, or at least about 98%, or at least about 99% sequence identity thereto.

Embodiment II-48. A self-inactivating viral-derived particle comprising
a) a viral capsid derived from an AAV serotype selected from the group consisting of AAV1, AAV2, AAV3, AAV4, AAV5, AAV6, AAV7, AAV8, AAV9, AAV10, AAV11, AAV12, AAV 44.9, AAV 9.45, AAV 9.61, AAV-Rh74, AAVRh10, and chimeras thereof;
b) 5′ and 3′ AAV ITR packaging components selected from the same serotype as the AAV capsid; and
c) the SIRV of any one of embodiments II-1 to II-47.

Embodiment II-49. A method of modifying a target nucleic acid sequence in a cell, comprising transfecting the cell with the SIRV of any one of embodiments II-1 to II-47, wherein the target nucleic acid sequence is modified by an RNP of the expressed Class 2 Type V protein and the first gRNA.

Embodiment II-50. The method of embodiment II-49, wherein the modifying comprises introducing a single-stranded break or a double-stranded break in the target nucleic acid sequence of the cell.

Embodiment II-51. The method of embodiment II-49, wherein the modifying comprises introducing an insertion, deletion, or mutation in the target nucleic acid sequence of the cell.

Embodiment II-52. The method of any one of embodiments II-49 to II-51, wherein the self-inactivating segment is cleaved by an RNP of the Class 2 Type V protein and the first gRNA subsequent to the modifying of the target nucleic acid sequence of the cell.

Embodiment II-53. The method of embodiment II-52, wherein the self-inactivating segment is cleaved at least 1 day, at least 2 days, at least 3 days, at least 4 days, at least 5 days, at least 6 days, or at least 7 days after the modifying of the target nucleic acid sequence.

Embodiment II-54. The method of any one of embodiments II-49 to II-53, wherein the cleavage of the self-inactivating segment results in reduced off-target modifying of nucleic acid sequence in the cell compared to a cell transduced with an SIRV not comprising the self-inactivating segments.

Embodiment II-55. The method of any one of embodiments II-49 to II-53, wherein the cleavage of the self-inactivating segment results in reduced or eliminated expression of the Class 2 Type V protein in the cell.

Embodiment II-56. A composition comprising:
a) an AAV expression cassette; and
b) a polynucleotide comprising sequences encoding one or more small hairpin RNA (shRNA) sequences, each operably linked to a promoter.

Embodiment II-57. The composition of embodiment II-56, wherein the AAV expression cassette comprises
a) a first adeno-associated virus (AAV) inverted terminal repeat (ITR) sequence;
b) a second AAV ITR sequence;
c) a sequence encoding a Class 2 Type V protein having a single RNA-guided RuvC domain;
d) a first promoter operably linked to the sequence encoding the Class 2 Type V protein;
e) a sequence encoding a first guide RNA (gRNA) comprising a scaffold sequence and a linked targeting sequence that is complementary to and capable of hybridizing with a target nucleic acid of a cell to be modified; and
f) a second promoter sequence operably linked to the sequence encoding the first gRNA.

Embodiment II-58. The composition of embodiment II-56 or II-57, wherein the polynucleotide comprises an encoding sequence for one, two, or three shRNA and linked promoters.

Embodiment II-59. The composition of any one of embodiments II-56 to II-58, wherein the shRNA encoding sequence comprises a sequence selected from the group consisting of SEQ ID NOS: 2640-2687, or a sequence having at least 85%, at least 90%, at least 95%, at least 95%, at least 96%, at least 97%, at least 98% identity thereto.

Embodiment II-60. The composition of any one of embodiments II-56, wherein the polynucleotide comprising the shRNA and linked promoters are linked exterior to the AAV transgene incorporated into a bacterial plasmid backbone.

Embodiment II-61. The composition of any one of embodiments II-56 to II-60, wherein the polynucleotide comprising the shRNA and linked promoters are inserted into
a) an AAV RepCap plasmid;
b) an AAV Helper plasmid; and/or
c) a separate vector.

Embodiment II-62. The composition of any one of embodiments II-57 to II-61, wherein the encoded Class 2, Type V protein comprises a sequence selected from the group consisting of SEQ ID NOS: 1-3, 49-321 and 2356-2488, or a sequence having at least 85%, at least 90%, at least 95%, at least 95%, at least 96%, at least 97%, at least 98%, or at least 99% identity thereto.

Embodiment II-63. The composition of any one of embodiments II-57 to II-62, wherein the first gRNA comprises a scaffold sequence selected from the group of sequences consisting of SEQ ID NOS: 2101-2331, 3992-3995, and 4028 or a sequence having at least 85%, at least 90%, at least 95%, at least 95%, at least 96%, at least 97%, at least 98% identity thereto.

Embodiment II-64. The composition of any one of embodiments II-57 to II-63, wherein the Class 2, Type V protein and gRNA encoding sequences are capable of being transcribed in a packaging cell transfected with the AAV expression cassette.

Embodiment II-65. The composition of any one of embodiments II-56 to II-64, wherein the shRNA is capable of being expressed and processed in a packaging cell transfected with the polynucleotide into a siRNA sequence complementary to and capable of hybridizing with an mRNA of the Class 2, Type V protein transcribed by the packaging cell.

Embodiment II-66. The composition of embodiment II-65, wherein the packaging cell is selected from the group consisting of baby hamster kidney (BHK), human embryonic kidney 293 (HEK293), HEK293T, NS0, SP2/0, YO myeloma cells, A549, P3X63 mouse myeloma cells, PER, PER.C6, NIH3T3, COS, HeLa, and Chinese hamster ovary (CHO).

Embodiment II-67. The composition of embodiment II-65 or II-66, wherein upon hybridization of the siRNA sequence to the mRNA of the Class 2, Type V protein, the Class 2, Type V protein mRNA is degraded such that expression of the Class 2, Type V protein is reduced or eliminated in the packaging cell.

Embodiment II-68. The composition of embodiment II-67, wherein expression of the Class 2, Type V protein is reduced by at least 40%, at least 50%, at least 60%, at least 70%, at least 80%, or at least 90% compared to a transfected packaging cell not comprising the shRNA, when assayed in a timed in vitro assay under comparable conditions.

Embodiment II-69. The composition of any one of embodiments II-57 to II-68, wherein the AAV expression cassette comprises
a) one or more self-inactivating segments comprising a protospacer adjacent motif (PAM) sequence and a polynucleotide sequence capable of being bound and cleaved by a ribonuclear protein complex (RNP) of the Class 2 Type V protein and a second gRNA;
b) a sequence encoding a second gRNA comprising a targeting sequence complementary to the self-inactivating segment; and
c) a third promoter operably linked to the second gRNA.

Embodiment II-70. The composition of embodiment II-69, wherein the second gRNA comprises a scaffold sequence selected from the group of sequences consisting of SEQ ID NOS: 2101-2331, 3992-3995, and 4028 or a sequence having at least 85%, at least 90%, at least 95%, at least 95%, at least 96%, at least 97%, at least 98% identity thereto.

Embodiment II-71. The composition of embodiment II-69 or II-70, wherein the one or more self-inactivating segments of the polynucleotide are located:
a) 5′ or 3′ adjacent to or within the sequence encoding the Class 2 Type V protein;
b) 5′ or 3′ adjacent to or within a Kozak sequence located between the first promoter and the sequence encoding the Class 2 Type V protein;
c) 5′ or 3′ adjacent to or within to the first promoter sequence;
d) 5′ or 3′ adjacent to or within the second promoter sequence;
e) 5′ or 3′ adjacent to or within the third promoter sequence;
f) 3′ downstream of the transcriptional start site for the sequence encoding the Class 2 Type V protein;
g) within one or more inserted introns in the polynucleotide encoding the Class 2 Type V protein;
h) at the 3′ end of the polynucleotide encoding the Class 2 Type V protein, between a stop codon and poly(A) termination site of the sequence encoding the Class 2 Type V protein; or
i) any combination of (a)-(h).

Embodiment II-72. The composition of any one of embodiments II-69 to II-71, wherein the self-inactivating segment comprises a 15-21 nucleotide sequence complementary to the targeting sequence of the second gRNA and that is 3′ adjacent to a PAM sequence recognized by an RNP of the Class 2 Type V protein and the second gRNA.

Embodiment II-73. The composition of any one of embodiments II-69 to II-72, wherein cleavage of the self-inactivating segments in a cell transfected with the composition by the RNP of the Class 2 Type V protein and the second gRNA results in reduced or eliminated expression of the Class 2 Type V protein or the gRNA encoded by the polynucleotide.

Embodiment II-74. The composition of any one of embodiments II-69 to II-73, wherein the PAM sequence of the one or more self-inactivating segments promotes less efficient cleavage or rate of cleavage of the self-inactivating segment by the RNP of the Class 2 Type V protein and the second gRNA compared to the PAM sequence 5′ and adjacent to the target nucleic acid of the cell to be modified.

Embodiment II-75. A method for reducing premature cleavage of a self-inactivating AAV (siAAV) transgene encoding a Class 2 Type V nuclease protein and one or more gRNAs in a packaging cell, comprising introducing a polynucleotide sequence encoding one or more small hairpin RNA (shRNA) into the packaging cell comprising the siAAV transgene, wherein the shRNA is capable of being expressed and processed into an siRNA sequence, and wherein the siRNA sequence is complementary to an mRNA of the Class 2 Type V nuclease transcribed by the packaging cell.

Embodiment II-76. The method of embodiment II-75, wherein the packaging cell is transfected with the siAAV transgene.

Embodiment II-77. The method of embodiment II-75 or II-76, wherein the transgene comprises
a) a first adeno-associated virus (AAV) inverted terminal repeat (ITR) sequence;
b) a second AAV ITR sequence;
c) a sequence encoding a Class 2 Type V protein having a single RNA-guided RuvC domain;
d) a first promoter operably linked to the sequence encoding the Class 2 Type V protein;
e) a sequence encoding a first guide RNA (gRNA) comprising a scaffold sequence and a linked targeting sequence that is complementary to and capable of hybridizing with a target nucleic acid of a cell to be modified; and
f) a second promoter sequence operably linked to the sequence encoding the first gRNA
g) a sequence encoding a second guide RNA (gRNA) comprising a scaffold sequence and a linked targeting sequence complementary to one or more self-inactivating segments of the transgene;
h) a third promoter sequence operably linked to the sequence encoding the second gRNA, wherein the third promoter has a sequence different from the sequence of the second promoter; and
i) one or more self-inactivating segments of the polynucleotide comprising a protospacer adjacent motif (PAM) sequence and a polynucleotide sequence capable of being bound and cleaved by a ribonuclear protein complex (RNP) of the Class 2 Type V protein and the second gRNA.

Embodiment II-78. The method of any one of embodiments II-75 to II-77, wherein the polynucleotide comprises an encoding sequence for one, two, or three shRNA and linked promoters.

Embodiment II-79. The method of any one of embodiments II-75 to II-78, wherein the shRNA encoding sequence comprises a sequence selected from the group consisting of SEQ ID NOS: 2640-2687, or a sequence having at least 85%, at least 90%, at least 95%, at least 95%, at least 96%, at least 97%, at least 98% identity thereto.

Embodiment II-80. The method of embodiment II-78 or II-79, wherein the polynucleotide comprising the shRNA and linked promoters are linked exterior to the AAV transgene inserted into a bacterial plasmid backbone.

Embodiment II-81. The method of any one of embodiments II-78 to II-80, wherein the polynucleotide comprising the shRNA and linked promoters are inserted into;
a) an AAV RepCap plasmid;
b) an AAV Helper plasmid; and/or
c) a separate vector.

Embodiment II-82. The method of any one of embodiments II-75 to II-81, wherein the packaging cell is selected from the group consisting of BHK, HEK293, HEK293T, NS0, SP2/0, YO myeloma cells, A549, P3X63 mouse myeloma cells, PER, PER.C6, NIH3T3, COS, HeLa, and CHO.

Embodiment II-83. The method of any one of embodiments II-75 to II-82, wherein upon transcription of the shRNA and Class 2 Type V Type V nuclease sequences, the shRNA is processed into siRNA which hybridizes with the mRNA of the Class 2 Type V nuclease and is degraded by the packaging cell.

Embodiment II-84. The method of embodiment II-83, wherein expression of the Class 2 Type V nuclease protein in the packaging cell is repressed by at least 40%, at least 50%, at least 60%, at least 70%, at least 80%, or at least 90% compared to a transfected packaging cell not comprising the shRNA sequence, when assayed in a timed in vitro assay under comparable conditions.

Embodiment II-85. The method of any one of embodiments II-75 to II-84, wherein the Class 2 Type V nuclease protein is a CasX comprising a sequence selected from the group consisting of SEQ ID NOS: 1-3, 49-321 and 2356-2488, or a sequence having at least 85%, at least 90%, at least 95%, at least 95%, at least 96%, at least 97%, at least 98%, or at least 99% identity thereto.

Embodiment II-86. The method of any one of embodiments II-77 to II-85, wherein the first and second gRNA each have a scaffold comprising a sequence selected from the group of sequences of SEQ ID NOS: 2101-2331, 3992-3995, and 4028 or a sequence having at least about 70%, at least about 80%, at least about 90%, at least about 95%, at least about 96%, at least about 97%, at least about 98%, at least about 99%, or 100% sequence identity thereto.

Embodiment II-87. The method of any one of embodiments II-77 to II-85, wherein the second guide comprises a sequence selected from the group consisting of SEQ ID NO: 2101-2238 and the first guide comprises a sequence selected from the group consisting of SEQ ID NOS: 2276-2296.

### EXAMPLES

### Example 1: Small CRISPR Proteins can Edit the Genome when Expressed from an AAV Episome In Vitro

This experiment demonstrated that small CRISPR proteins (such as CasX) can edit a genome when expressed from an AAV plasmid or an AAV vector in vitro.

### Materials and Methods

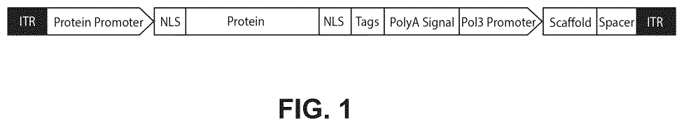

The AAV transgene between the ITRs was broken into different parts, which consisted of the therapeutic cargo and accessory elements relevant to expression of the therapeutic cargo in mammalian cells. AAV vectors were designed, built, and tested in both plasmid and AAV form in mammalian cells. A schematic of a representative AAV transgene and one configuration of its components is shown in FIG. 1.

In this example, three plasmids were constructed (AAV construct 1, AAV construct 2, and AAV construct 3; see Table 64 for component sequences), where the only difference in the plasmid sequence between the ITRs was in the affinity tag region.

### Cloning and Quality Control (QC):

AAV vectors were cloned using a 4-part Golden Gate Assembly consisting of a pre-digested AAV backbone, small CRISPR protein-encoding DNA, and flanking 5′ and 3′ DNA sequences. 5′ sequences contained enhancer, protein promoter and N-terminal NLS, while 3′ sequences contained C-terminal NLS, Woodchuck Hepatitis Virus (WHV) Posttranscriptional Regulatory Element (WPRE), poly(A) signal, RNA promoter and guide RNA containing spacer 12.7, targeting tdTomato (DNA sequence: CTGCATTCTAGTTGTGGTTT, SEQ ID NO: 462). 5′ and 3′ parts were ordered as gene fragments, PCR-amplified, and assembled into AAV vectors through cyclical Golden Gate reactions using T4 Ligase and BbsI.

Assembled AAV vectors were then transformed into chemically-competent E. coli (Stbl3s). Transformed cells were recovered for 1 hour in a 37° C. shaking incubator, plated on Kanamycin LB-Agar plates and allowed to grow at 37° C. for 12-16 hours. Colony PCR was performed to determine clones that contained full transgenes. Correct clones were inoculated in 50 mL of LB media with kanamycin and grown overnight. Plasmids were then midi-prepped the following day and sequence-verified. To assess the quality of midipreps, constructs were processed in restriction digests with XmaI (which cuts in each of the ITRs) and XhoI (which cuts once in the AAV genome). Digests and uncut constructs were then run on a 1% agarose gel and imaged on a ChemiDoc. If the plasmid was >90% supercoiled, the correct size, and the ITRs were intact, the construct was tested via nucleofection and/or transduction.

### Method for Plasmid Nucleofection:

Plasmids containing the AAV genome were transfected in a mouse immortalized neural progenitor cell line isolated from the Ai9-tdTomato mouse (tdTomato mNPCs) using the Lonza P3 Primary Cell 96-well Nucleofector Kit. Briefly, Ai9 is a Cre reporter tool strain designed to have a loxP flanked STOP cassette preventing the transcription of a CAG promoter-driven tdTomato marker. Ai9 mice, or Ai9 mNPCs, express tdTomato following Cre-mediated recombination to remove the STOP cassette. Sequence-validated plasmids were diluted to concentrations of 200 ng/μl, 100 ng/μl, 50 ng/μL and 25 ng/μL, and 5 μL of each (1000 ng, 500 ng, 250 ng and 125 ng) were added to P3 solution containing 200,000 tdTomato mNPCs. The combined solution was nucleofected using a Lonza 4D Nucleofector System following program EH-100. Following nucleofection, the solution was quenched with pre-equilibrated mNPC medium (DMEM/F12 with GlutaMax™, 10 mM HEPES, 1×MEM Non-Essential Amino Acids, 1× penicillin/streptomycin, 1:1000 2-mercaptoethanol, 1×B-27 supplement, minus vitamin A, 1×N2 with supplemented growth factors bFGF and EGF (20 ng/mL final concentration). The solution was then aliquoted in triplicate (approx. 67,000 cells per well) in a 96-well plate coated with PLF (1×Poly-DL-ornithine hydrobromide, 10 mg/mL in sterile diH₂O, 1× laminin, and 1× fibronectin). 48 hours after transfection, treated cells were replenished with fresh mNPC media containing growth factors. 5 days after transfection, tdTomato mNPCs were lifted and activity was assessed by FACS.

### AAV Production:

Suspension HEK293T cells were adapted from parental HEK293T and grown in FreeStyle 293 media. For screening purposes, small scale cultures (20-30 mL cultured in 125 mL Erlenmeyer flasks and agitated at 110 rpm) were diluted to a density of 1.5e+6 cells/mL on the day of transfection. Endotoxin-free pAAV plasmids with the transgene flanked by ITR repeats were co-transfected with plasmids supplying the adenoviral helper genes for replication and AAV rep/cap genome using PEIMax® (Polysciences) in serum-free OPTI-MEM® media. Cultures were supplemented with 10% CDM4HEK293 (HyClone) 3 hours post-transfection. Three days later, cultures were centrifuged at 1000 rpm for 10 minutes to separate the supernatant from the cell pellet. The supernatant was mixed with 40% PEG 2.5M NaCl (8% final concentration) and incubated on ice for at least 2 hours to precipitate AAV viral particles. The cell pellet, containing the majority of the AAV vectors, was resuspended in lysis media (0.15 M NaCl, 50 mM Tris HCl, 0.05% Tween, pH 8.5), sonicated on ice (15 seconds, 30% amplitude) and treated with Benzonase (250 U/μL, Novagen) for 30 minutes at 37° C. Crude lysate and PEG-treated supernatant were then centrifuged at 4000 rpm for 20 minutes at 4° C. to resuspend the PEG precipitated AAV (pellet) with cell debris-free crude lysate (supernatant), and then clarified further using a 0.45 μM filter.

To determine the viral genome titer, 1 μL from crude lysate containing viruses was digested with DNase and ProtK, followed by quantitative PCR. 5 μL of digested virus was used in a 25 μL qPCR reaction composed of IDT primetime master mix and a set of primer and 6′FAM/Zen/IBFQ probe (IDT) designed to amplify the CMV promoter region (Fwd 5′-CATCTACGTATTAGTCATCGCTATTACCA-3′ (SEQ ID NO: 456); Rev 5′-GAAATCCCCGTGAGTCAAACC-3′ (SEQ ID NO: 457), Probe 5′-TCAATGGGCGTGGATAG-3′; SEQ ID NO: 458) or a 62 nucleotide-fragment located in the AAV2-ITR (Fwd 5′-GGAACCCCTAGTGATGGAGTT-3′ (SEQ ID NO: 459); Rev 5′-CGGCCTCAGTGAGCGA-3′ (SEQ ID NO: 460), Probe 5′-CACTCCCTCTCTGCGCGCTCG-3′; SEQ ID NO: 461). Ten-fold serial dilutions (5 μl each of 2e+9 to 2e+4 DNA copies/mL) of an AAV ITR plasmid were used as reference standards to calculate the titer (viral genome (vg)/mL) of viral samples. The qPCR program was set up as: initial denaturation step at 95° C. for 5 minutes, followed by 40 cycles of denaturation at 95° C. for 1 min and annealing/extension at 60° C. for 1 min.

### AAV Transduction:

10,000 cells/well of mNPCs were seeded on PLF-coated wells in 96-well plates 48-hours before AAV transduction. All viral infection conditions were performed in triplicate, with normalized number of vg among experimental vectors, in a series of 3-fold dilution of multiplicity of infection (MOI) ranging from about 1.0e+4 to 1.0e+6 vg/cell. Calculations were based on an estimated number of 20,000 cells per well at the time of transfection. Final volumes of 50 μL of AAV vectors diluted in pre-equilibrated mNPC medium supplemented with bFGF/EGF growth factors (20 ng/ml final concentration) were applied to each well. 48 hours post-transfection, a complete media change was performed with fresh media supplemented with growth factors. Editing activity (tdT+ cell quantification) was assessed by FACS 5 days post-transfection.

### Method for Assessing Activity by FACS:

Five days after transfection, treated tdTomato mNPCs in 96-well plates were washed with dPBS and treated with 50 μL TrypLE for 15 minutes. Following cell dissociation, treated wells were quenched with media containing DMEM, 10% FBS and 1× penicillin/streptomycin. Resuspended cells were transferred to round-bottom 96-well plates and centrifuged for 5 min at 1000×g. Cell pellets were then resuspended with dPBS containing 1×DAPI, and plates were loaded into an Attune™ NxT Flow Cytometer Autosampler. The Attune™ NxT flow cytometer was run using the following gating parameters: FSC-A×SSC-A to select cells, FSC-H×FSC-A to select single cells, FSC-A×VL1-A to select DAPI-negative alive cells, and FSC-A×YL1-A to select tdTomato positive cells.

### Results:

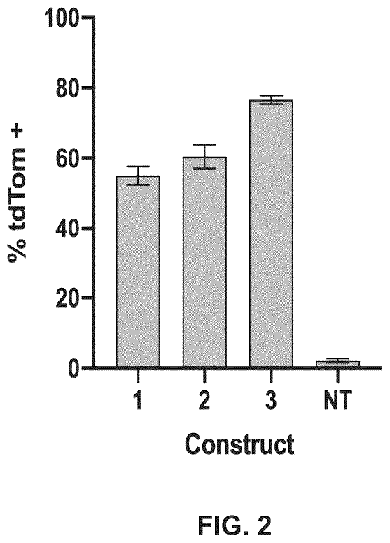

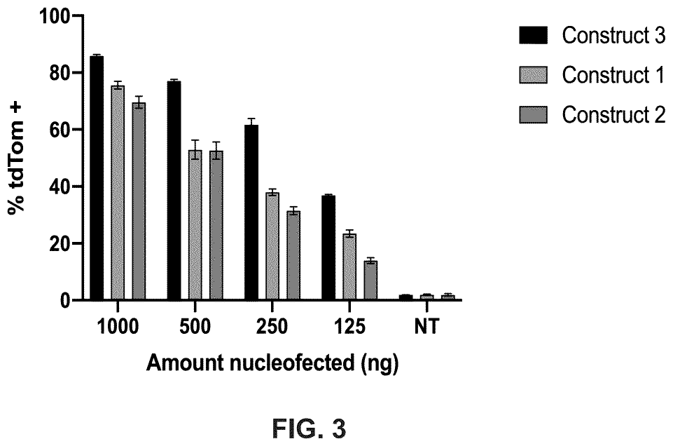

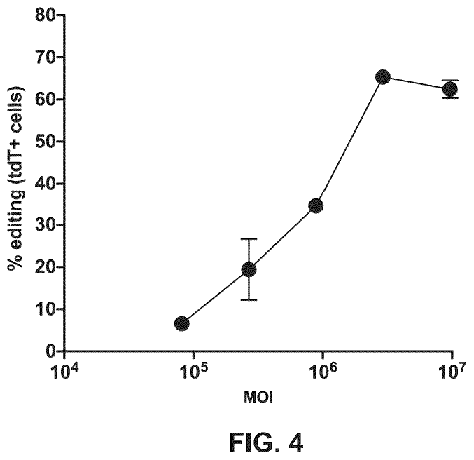

The results in graph in FIG. 2 show that CasX variant 491 and guide variant 174 with a spacer targeting the tdTomato stop cassette (spacer 12.7, with sequence CTGCATTCTAGTTGTGGTTT; SEQ ID NO: 462), when delivered by nucleofection of an AAV transgene plasmid, was able to edit the target stop cassette in mNPCs (measured by percentage of cells that are tdTom+ by FACS). Among the vectors tested, CasX 491.174 delivered in construct AAV.3 (with 80% tdTomato+ cells) outperformed the others. FIG. 3 shows that all three vectors tested achieved editing at the tdTomato locus in a dose-dependent manner. FIG. 4 shows results of editing using AAV construct 3 in an AAV vector, which demonstrated a dose-dependent response, achieving a high degree of editing.

The experiments demonstrate that small CRISPR proteins (such as CasX) and targeted guides can edit the genome when expressed from an AAV transgene plasmid or episome in vitro.

### Example 2: Packaging of Small CRISPR Systems within an AAV Vector

This experiment demonstrated that small CRISPR proteins (such as CasX) and gRNA can be encoded and efficiently packaged within an AAV vector.

### Materials and Methods

AAV vectors were generated using standard methods for AAV production, purification and characterization, as described in Example 1. For characterization, AAV viral genome titer was measured by qPCR, and the empty-full ratio was quantified using scanning transmission electron microscopy (STEM).

### Results:

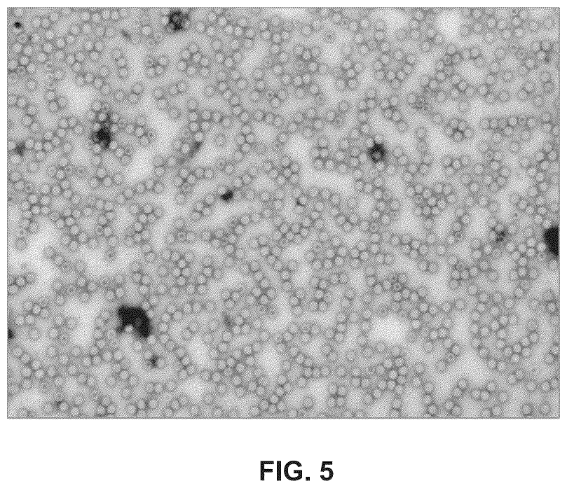

The genomic DNA titer (by qPCR) for this AAV preparation was measured to be 6e12 vg/mL, and was generated from 1L of HEK293T cell culture. FIG. 5 is an image from a scanning transmission electron microscopy (STEM) micrograph showing that an estimated 90% of the particles in this AAV formulation contained viral genomes; i.e., were loaded with the CRISPR cargo. These results demonstrate that sequences encoding CasX variant proteins and gRNA can be efficiently packaged in an AAV vector, resulting in high titers and high packaging efficiency.

### Example 3: In Vivo Editing of a Genome with Small CRISPR Proteins Expressed from an AAV Episome

This experiment demonstrates that small CRISPR proteins (such as CasX) and gRNA are capable of being delivered by AAV and can edit the genome when expressed from an AAV episome in vivo.

### Materials and Methods

AAV vectors were generated using standard methods for AAV production, purification and characterization, as described in Example 1.

In vivo AAV administration and tissue processing:

P0-P1 pups from Ai9 mice were injected with AAV with a transgene encoding CasX variant 491 and guide variant 174 with spacer 12.7. Briefly, mice were cryo-anesthetized and 1-2 μL of AAV vector (˜1e11 viral genomes (vg)) was unilaterally injected into the intracerebroventricular (ICV) space using a Hamilton syringe (10 μL, Model 1701 RN SYR Cat No: 7653-01) fitted with a 33-gauge needle (small hub RN NDL—custom length 0.5 inches, point 4 (45 degrees)). Post-injection, pups recovered on a warm heating pad before being returned to their cages. 1 month after ICV injections, animals were terminally anesthetized with an intraperitoneal injection of ketamine/xylazine, and perfused transcardially with saline and fixative (4% paraformaldehyde). Brains were dissected and further post-fixed in 4% paraformaldehyde (PFA), followed by infiltration with 30% sucrose solution, and embedding in OCT compound. OCT-embedded brains were coronally sectioned using a cryostat. Sections were then mounted on slides, counter-stained with DAPI to label cell nuclei, coverslips were added, and the slides were imaged on a fluorescence microscope. Images were processed using ImageJ software and editing levels were quantified by counting the number of tdTom+ cells as a percentage of DAPI-labeled nuclei.

In a subsequent experiment to assess editing in peripheral tissues, particularly in the liver and in the heart, P0-P1 pups from Ai9 mice were cryo-anesthetized and were intravenously injected with ˜1e12 viral genomes (vg) of the same AAV construct in a 40 μL volume. Post-injection, pups recovered on a warm heating pad before being returned to their cages. 1-month post-administration, animals were terminally anesthetized and heart and liver tissues were necropsied and processed as described above.

### Results:

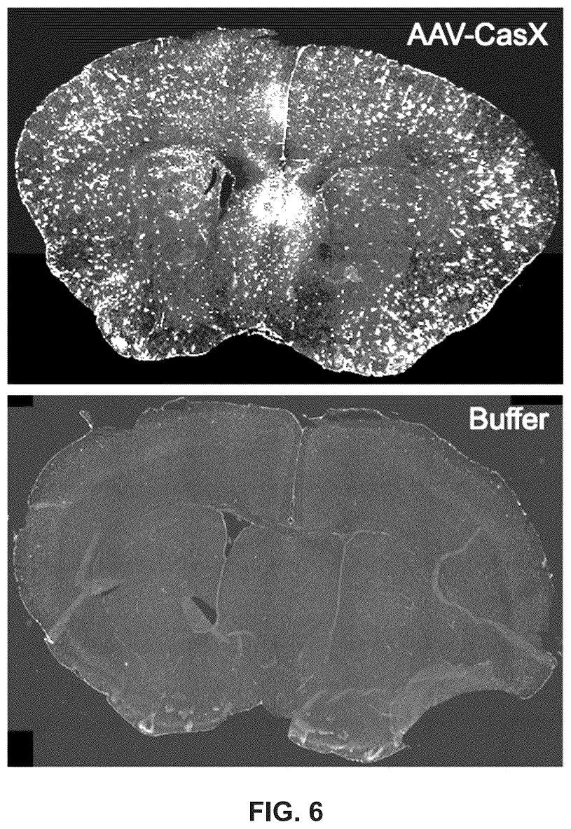

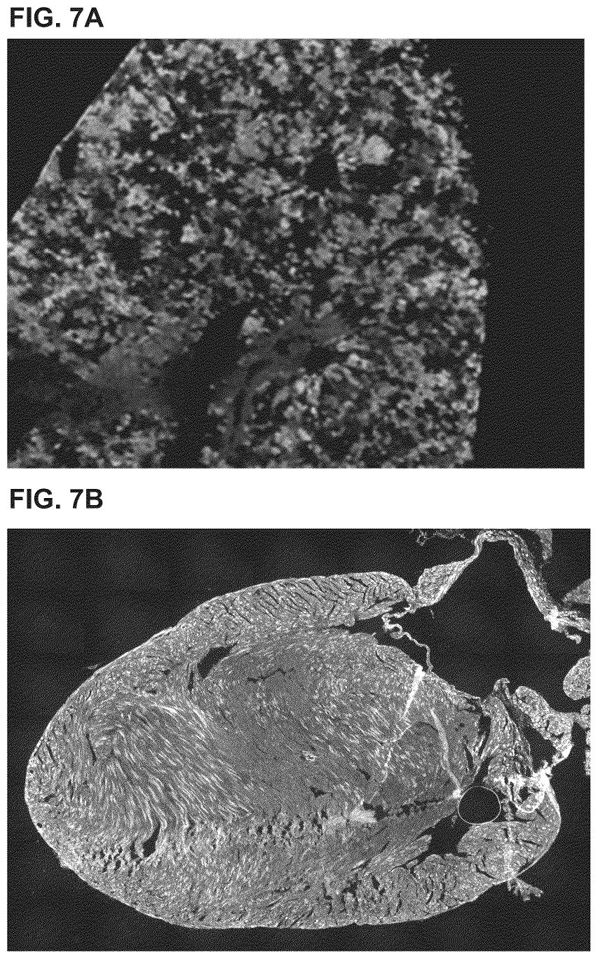

FIG. 6 provides comparative immunohistochemistry (IHC) images of brain tissue processed from an Ai9 mouse that received an ICV injection of AAV packaging CasX variant 491 and guide scaffold 174 with spacer 12.7. The tissue was stained with 4′,6-diamidino-2-phenylindole (top panel). The signal from cells in the tdTom channel indicates that the tdTom locus within these cells was successfully edited. The tdTom+ cells (in white) are distributed evenly across all regions of the brain, indicating that ICV-administered AAV carrying CasX, guide and spacer were able to reach and edit these cells as compared to a buffer control (bottom panel). The images are representative of those obtained from 3 mice for each group. Additionally, the results presented in FIG. 7A (liver) and FIG. 7B (heart) demonstrate that, under the conditions of the experiments, the AAV were able to distribute within the liver and the heart (edited cells in white) and edit the genome when expressed from single AAV episomes in vivo.

The results demonstrate that AAV encoding small CRISPR proteins (such as CasX) and a targeting guide can distribute within the tissues, when delivered either locally (brain) or systemically and edit the target genome when expressed from single AAV episomes in vivo.

### Example 4: Protein Promoter Selection Enhances AAV Vector Potency

This experiment demonstrated that small CRISPR protein expression and editing, can be enhanced by utilizing different promoters in an AAV construct for the encoded protein. Cargo space in the AAV transgene can be maximized with the use of short promoters in combination with small CRISPR proteins such as CasX. Additionally, these experiments demonstrate that expression can be enhanced with the use of promoters that would otherwise be too long to be efficiently packaged in AAV vector, if they were combined with larger CRISPR proteins, such as Cas9. The use of long, cell-type-specific promoters to enhance small CRISPR proteins is an advantage to the AAV system, and not possible in traditional CRISPR systems due to the size of traditional CRISPR proteins.

### Materials and Methods

Cloning and molecular biology methods were conducted as described in Example 1. Promoter variants (Table 10) were cloned upstream of CasX protein in an AAV-cis plasmid. The sequences of the additional components of the AAV constructs, with the exception of sequences encoding the CasX (Table 63) and the one or more gRNA (Table 26), are listed in Table 64.

TABLE 10
Promoter variant sequences

| AAV Construct | Promoter | Size | SEQ |
|---|---|---|---|
| ID | based on | (bp) | ID NO |
| 1, 2, 3, 7, 44 | CMV | 584 | 463 |
| 4 | UbC | 400 | 464 |
| 5 | EFS | 234 | 465 |
| 6 | CMV-s | 335 | 466 |
| 8 | CMVd1 | 100 | 467 |
| 9 | CMVd2 | 52 | 468 |
| 10 | miniCMV | 39 | 469 |
| 11, 26 | HSVTK | 146 | 470 |
| 12 | miniTK | 63 | 471 |
| 13 | miniIL2 | 114 | 472 |
| 14 | GRP94 | 710 | 473 |
| 15 | Supercore 1 | 81 | 474 |
| 16 | Supercore 2 | 81 | 475 |
| 17 | Supercore 3 | 81 | 476 |
| 18 | Mecp2 | 229 | 477 |
| 19 | CMVmini | 68 | 478 |
| 20 | CMVmini2 | 65 | 479 |
| 21 | miniCMVIE | 39 | 480 |
| 22 | adML | 81 | 481 |
| 23 | hepB | 107 | 482 |
| 54 | RSV | 227 | 483 |
| 55 | hSyn | 448 | 484 |
| 56 | SV40 | 330 | 485 |
| 57 | hPGK | 551 | 486 |
| 58 | JeT | 164 | 487 |
| 59 | JeT + UsP intron | 326 | 488 |
| 60 | hRLP30 | 325 | 489 |
| 61 | hRPS18 | 243 | 490 |
| 62 | CBA | 493 | 491 |
| 63 | CBH | 565 | 492 |
| 64 | CMV core | 204 | 493 |

### Method for Plasmid Nucleofection:

Immortalized neural progenitor cells were nucleofected as described in Example 1. Sequence-validated plasmids were diluted to concentrations of 200 ng/ul, 100 ng/ul, 50 ng/μL and 25 ng/μL, and 5 μL of each (1000 ng, 500 ng, 250 ng and 125 ng) were added to P3 solution containing 200,000 tdTomato mNPCs.

AAV viral production and characterization, and AAV transduction and editing level assessment in mNPTC-tdT cells by FACS were conducted as described in Example 1.

### Results:

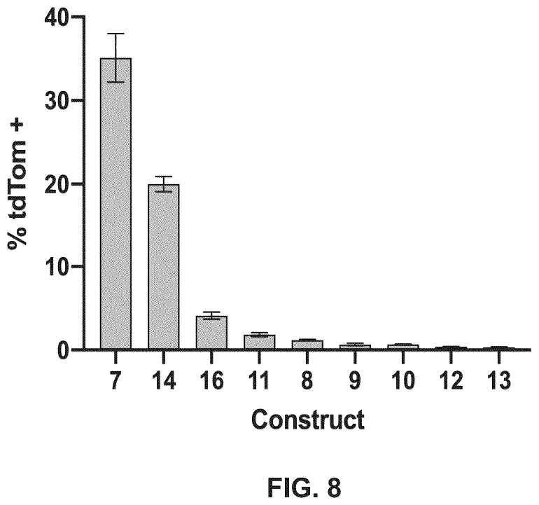

The results shown in FIG. 8 demonstrate that several different promoters with CasX protein 438, scaffold variant 174 and spacer targeting the tdTomato stop cassette (spacer 12.7, with sequence CTGCATTCTAGTTGTGGTTT, SEQ ID NO: 462), when delivered by nucleofection of AAV transgene plasmid, were able to edit the target stop cassette in mNPCs at a dose of 1000 ng. These promoters ranged in length from over 700 nucleotides to as short as 81 nucleotides (Table 10, the promoters used correspond to the construct ID numbers in the left-most column). Among the promoters tested, constructs AAV7 (CMV promoter) and AAV14 (GRP94 promoter) showed considerable editing potency.

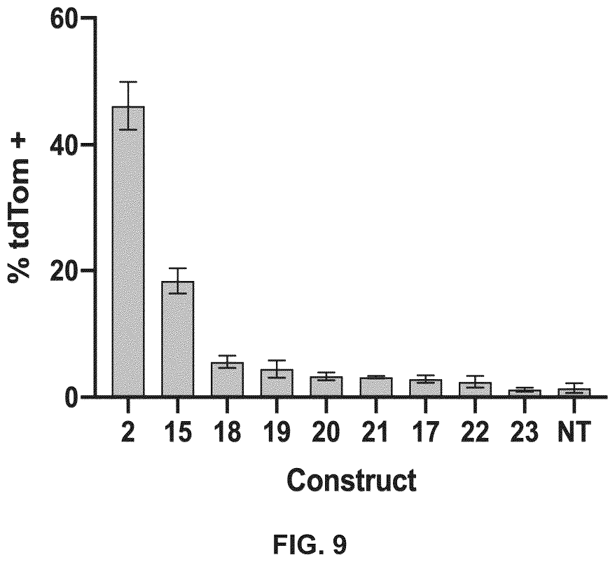

The results shown in FIG. 9 demonstrate that several short promoters combined with CasX variant 491, scaffold variant 174 and spacer 12.7, when delivered by nucleofection of AAV transgene plasmid, edit the target stop cassette in mNPCs at a dose of 500 ng. Other than construct AAV.2, which had a promoter of 584 nucleotides, all constructs had promoters that were less than 250 nucleotides in length. Among the protein promoters tested, construct AAV15 showed considerable editing potency, especially given its short length (81 nucleotides).

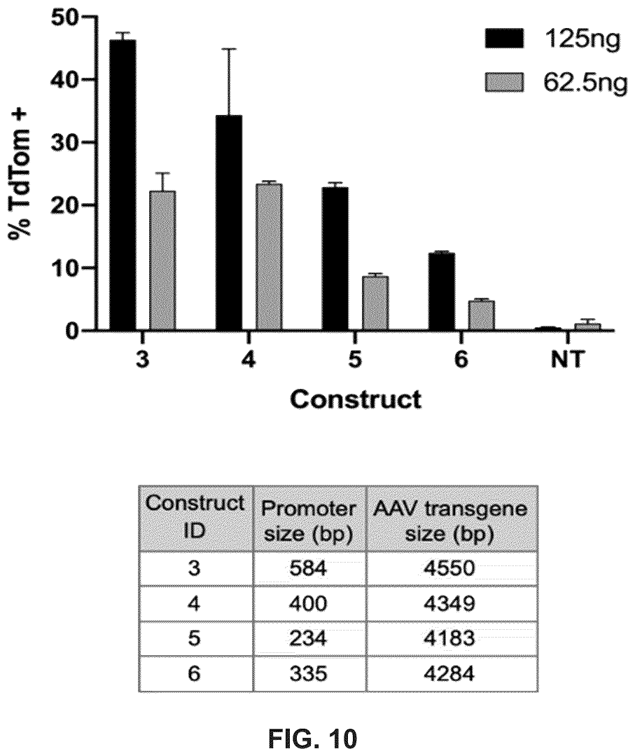

The results shown in FIG. 10 demonstrate that four promoters with CasX variant 491 and scaffold variant 174 with spacer 12.7, when delivered by nucleofection of AAV transgene plasmid, edit the target stop cassette in mNPCs at doses of 125 ng and 62.5 ng. Constructs AAV4, AAV5 and AAV6 have promoter lengths less than or equal to 400 nucleotides, and thus may maximize editing potency while minimizing AAV cargo capacity.

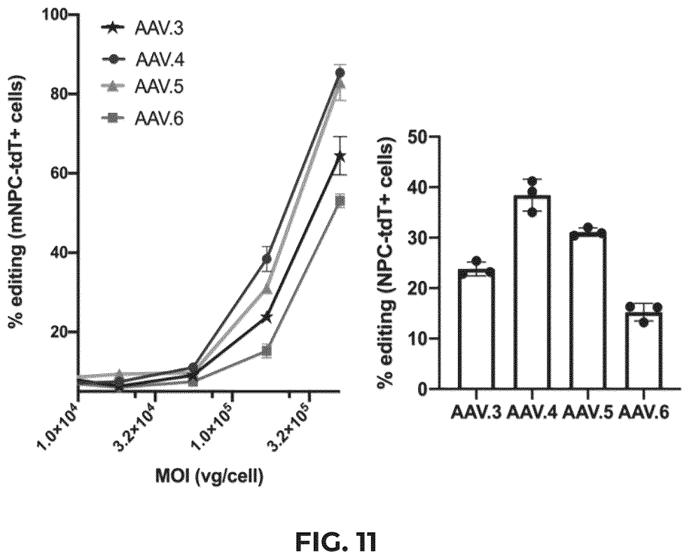

The results shown in FIG. 11 demonstrate that use of four promoter variants in the AAV also result in robust editing. Briefly, AAVs with transgene constructs AAV.3 (CMV), AAV.4 (UbC), AAV.5 (EFS) and AAV.6 (CMV-s) were generated. Each construct showed dose-dependent editing at the target locus (FIG. 11, left panel). At an MOI of 2e5, AAV.4 showed editing at 38%±3% at the target locus, outperforming the other constructs (FIG. 11, right panel).

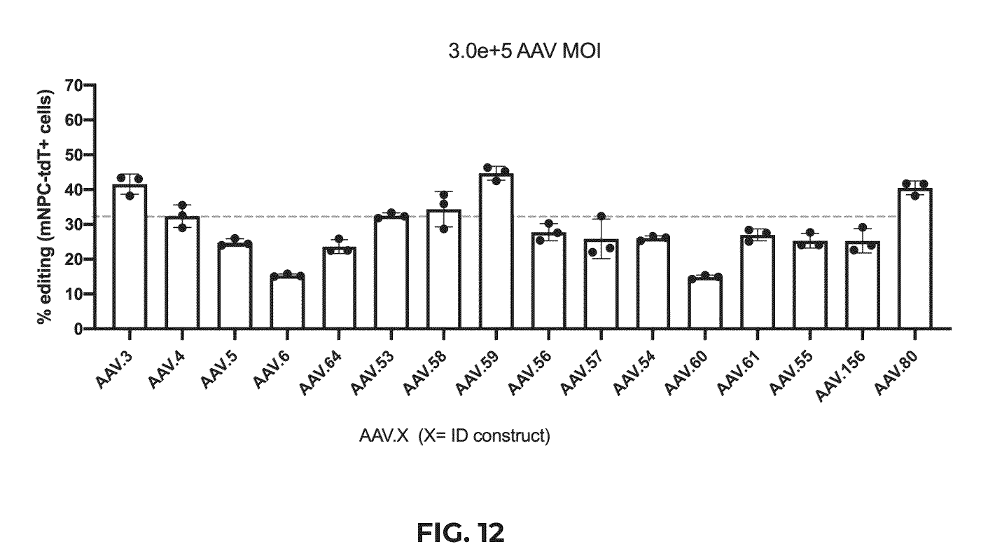

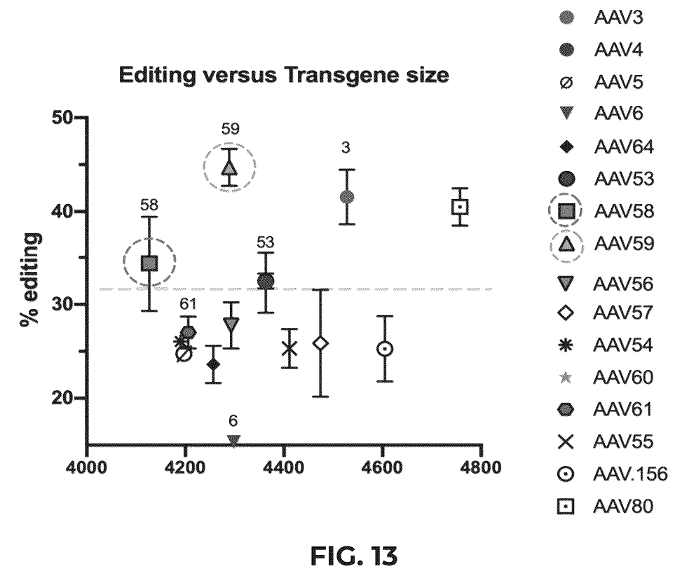

In the experiments performed for the results portrayed in FIG. 12, several new protein promoters were compared against the top 4 protein promoter variants identified previously (AAV.3, AAV.4, AAV.5 and AAV.6). Briefly, AAVs were generated with corresponding transgene constructs indicated by the ID numbers in FIG. 12, and transduced in tdTomato mNPCs. At an MOI of 3e5, 5 days after transduction, multiple promoters displayed improved editing (FIG. 12). In particular, constructs AAV.58 (JeT) and AAV.59 (JeT+UsP intron) had editing activity above 30% while minimizing transgene size (see FIG. 13 for a summary). Constructs AAV.58 and AAV.59 contained promoters that are 420 and 258 bp smaller, respectively, than construct AAV.3, yet resulted in similar or improved editing of the target locus. In particular, inclusion of an intron in the promoter of construct AAV.59 led to increased editing compared to construct AAV.58, which lacked the intron, demonstrating that the inclusion of introns in the AAV construct promoters is beneficial.

The results demonstrate that expression of small CRISPR proteins (such as CasX) can be enhanced by utilizing long promoters that would otherwise be unusable in AAV constructs with traditional CRISPR proteins due to the size constraints of the AAV genome. Furthermore, combining short promoters with small CRISPR proteins (such as CasX) allows for significant reductions in AAV transgene cargo without compromising expression efficiency. This conservation of space allows for the inclusion of additional accessory elements, such as enhancers and regulatory elements in the transgene, which would enable increased editing potential.

### Example 5: Potency of Small CRISPR Systems is Enhanced by AAV RNA Promoter Choice

Experiments were performed to demonstrate that the editing potency of small CRISPR systems, such as CasX, can be enhanced if certain promoters are chosen for expression of the gRNA, which recognizes target DNA for editing, in an AAV vector. By using RNA promoters with different strengths, guide RNA expression can be modulated, which affects editing potency. The AAV platform based on the CasX system provides enough cargo space in the AAV to include at least 2 independent promoters for the expression of two incorporated guide RNAs. By combining different promoters, expression of multiple guide RNAs can be tuned within a single AAV transgene. Engineering shorter versions of RNA promoters that still retain editing potency also results in increased space in the vector for the inclusion of other accessory elements in the AAV transgene.

### Materials and Methods

The methods of Example 1 were used for cloning and quality control of the constructs, as well as for plasmid nucleofection and AAV production, transduction, and FACS analysis. The sequences of the Pol III promoters are presented in Table 11. The sequences of the additional components of AAV constructs, with the exception of sequences encoding the CasX (Table 63) and the one or more gRNA (Table 26), are listed in Table 64.

TABLE 11
Sequences of engineered Pol III promoters

| SEQ | AAV construct | Pol III | Promoter size |
|---|---|---|---|
| ID NO: | ID | promoter | (bp) |
| 661 | 3, 53, 157 | hU6 isoform 1 | 241 |
| 662 | 32, 158 | H1 | 215 |
| 496 | 33 | 7SK | 267 |
| 497 | 85/89 | hU6 variant 1 | 103 |
| 498 | 86 | hU6 variant 2 | 38 |
| 499 | 87 | hU6 variant 3 | 67 |
| 500 | 88 | hU6 variant 4 | 79 |
| 501 | 90 | hU6 variant 5 | 111 |
| 502 | 91 | hU6 variant 6 | 127 |
| 503 | 92 | hU6 variant 7 | 123 |
| 504 | 93 | hU6 variant 8 | 143 |
| 505 | 94 | hU6 variant 9 | 131 |
| 506 | 95 | hU6 variant 10 | 159 |
| 507 | 96 | hU6 variant 11 | 103 |
| 508 | 97 | hU6 variant 12 | 111 |
| 509 | 98 | hU6 variant 13 | 127 |
| 510 | 99 | hU6 variant 14 | 103 |
| 511 | 100 | hU6 variant 15 | 131 |
| 512 | 101 | hU6 variant 16 | 159 |
| 513 | 102 | hU6 variant 17 | 128 |
| 2688 | 159 | H1 core | 91 |
| 2689 | 160 | H1 core + 7SK hybrid 1 | 92 |
| 2690 | 161 | H1 core + 7SK hybrid 2 | 92 |
| 2691 | 162 | H1 core + 7SK hybrid 3 | 91 |
| 2692 | 163 | H1 core + 7SK hybrid 4 | 91 |
| 2693 | 164 | H1 core + 7SK hybrid 5 | 92 |
| 2694 | 165 | H1 core + 7SK hybrid 6 | 91 |
| 2695 | 166 | H1 core + 7SK hybrid 7 | 91 |
| 2696 | 167 | H1 core + 7SK hybrid 8 | 91 |
| 2697 | 168 | H1 core + 7SK hybrid 9 | 92 |
| 2698 | 169 | H1 core + U6 hybrid 1 | 91 |
| 2699 | 170 | H1 core + U6 hybrid 2 | 94 |
| 2700 | 171 | H1core + 7SK + U6 hybrid 1 | 92 |
| 2701 | 172 | H1 core + U6 hybrid 3 | 90 |
| 2702 | 173 | H1 core + 7SK + U6 hybrid 2 | 94 |
| 2703 | 174 | H1 core + 7SK + U6 hybrid 3 | 94 |
| 2704 | — | hU6 isoform 2 | 249 |
| 2705 | — | hU6 isoform 3 | 249 |
| 2706 | — | hU6 isoform 4 | 249 |
| 2707 | — | hU6 isoform 5 | 249 |
| 2708 | — | mU6 | 304 |
| 3996 | — | mU6 isoform | 314 |

### Results:

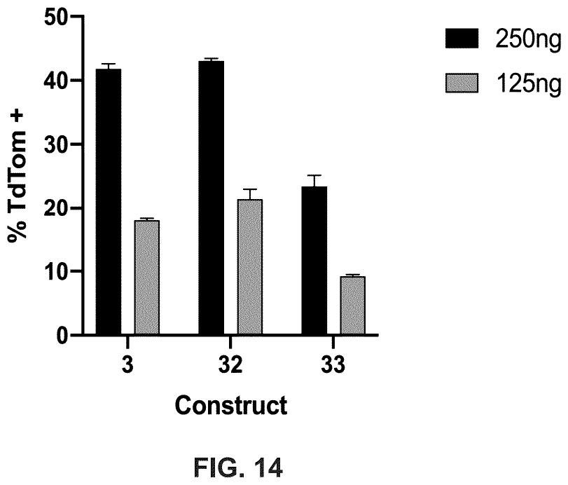

The results portrayed in FIG. 14 demonstrate that AAV vectors using three distinct RNA promoters, in combination with CasX protein 491, scaffold variant 174 and spacer 12.7, when delivered by nucleofection of the AAV transgene plasmid, edit the target stop cassette in mNPCs at doses of 250 ng and 125 ng. Constructs AAV3 (U6 promoter) and AAV32 (H1 promoter) have similar activity, editing at the target locus with 42% efficiency. Construct 33 (7SK promoter) shows ˜56% of the activity of constructs AAV3 and AAV32.

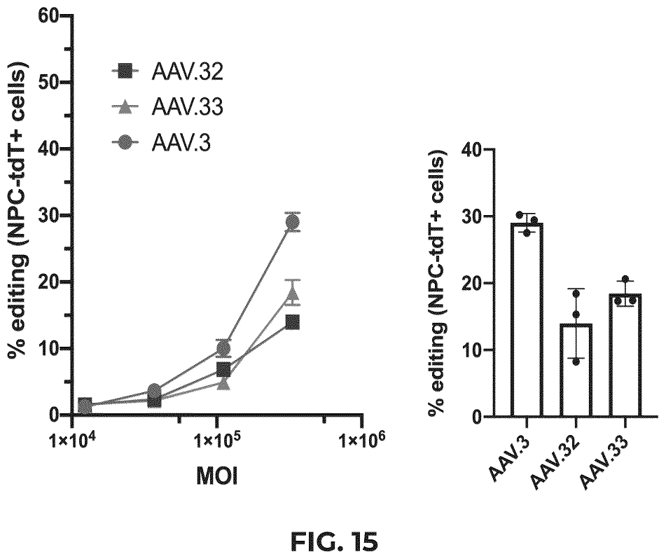

The results portrayed in FIG. 15 demonstrate that the same three distinct promoters, in combination with CasX protein 491, scaffold variant 174 and spacer 12.7, when delivered as AAV, edit the target stop cassette in mNPCs. AAV.3, AAV.32, AAV.33 were generated with transgene constructs 3, 32 and 33 respectively. Each vector displayed dose-dependent editing at the target locus (FIG. 15, left panel). At an MOI of 3e5, AAV.32 and AAV.33 had 50-60% of the potency of AAV.3 (FIG. 15, right panel).

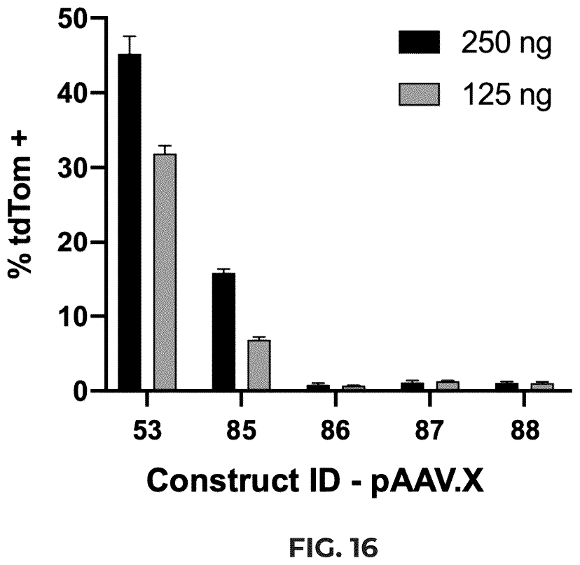

The results shown in FIG. 16 demonstrate that constructs having one of four different truncations of the U6 promoter, in combination with CasX protein 491, scaffold variant 174 and spacer 12.7, when delivered by nucleofection of the AAV transgene plasmid, were each able to edit the target stop cassette at different levels in mNPCs at doses of 250 ng and 125 ng. Construct AAV85 (hU6 variant 1) had 33% of the potency of the base construct AAV53 (hU6), while constructs AAV86 (hU6 variant 2), AAV87 (hU6 variant 3) and AAV88 (hU6 variant 4) did not show any editing and were comparable to a non-targeting control.

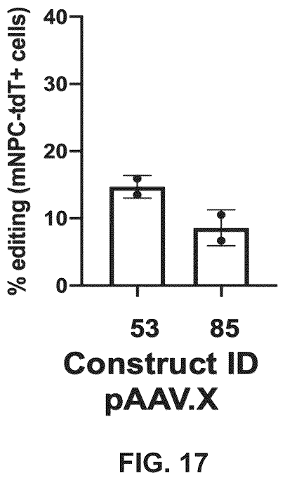

FIG. 17 presents results of an experiment comparing editing in mNPCs between AAV generated with base construct AAV53 (hU6 promoter) to AAV generated with construct AAV85 (hU6 variant 1). When delivered as AAV, AAV.85 was able to edit at 7% compared to 15% for AAV.53 at an MOI of 3e5, consistent with the results from FIG. 16.

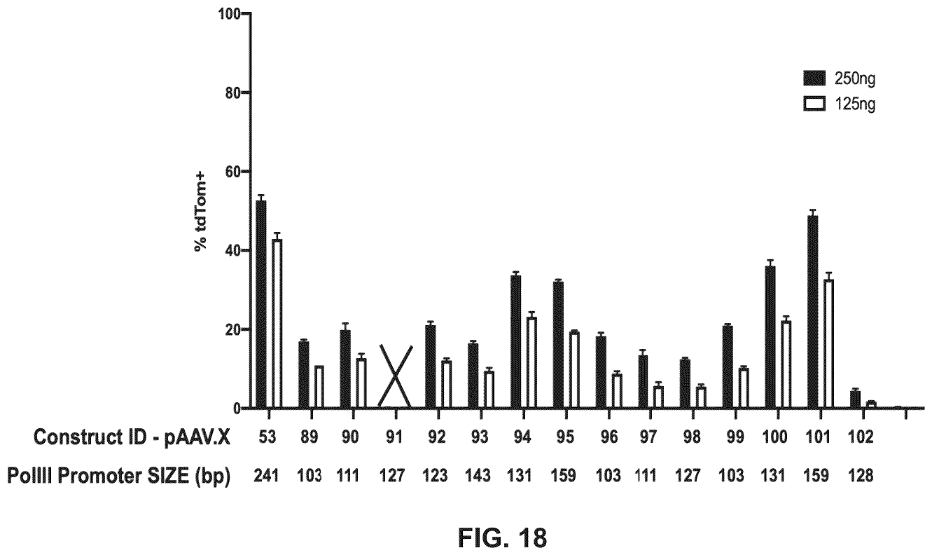

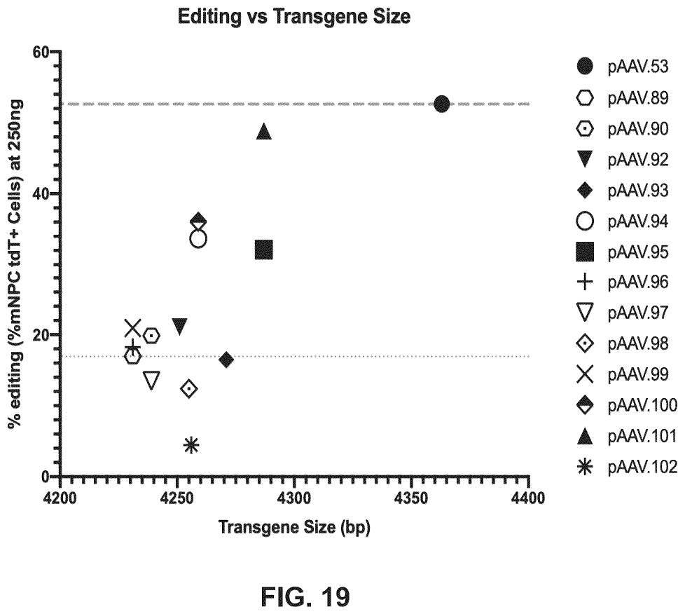

The results of FIG. 18 demonstrate that constructs with engineered U6 promoters were able to edit the target stop cassette at differential levels in mNPCs at doses of 250 ng and 125 ng. Engineered U6 promoters were designed to minimize the size of the promoter relative to the base U6 promoter. Construct AAV.53 carried the hU6 promoter, in combination with encoded CasX protein 491, scaffold variant 174 and spacer 12.7, and the constructs with the variant promoters carried the same CasX, scaffold and spacer as AAV.53. Constructs were delivered to mNPCs by nucleofection of AAV transgene plasmid, and were able to edit the target stop cassette at different levels in mNPCs at doses of 250 ng and 125 ng. One cluster of constructs (AAV.89 (hU6 variant 1), 90 (hU6 variant 5), 92 (hU6 variant 7), 93 (hU6 variant 8), 96 (hU6 variant 11), 97 (hU6 variant 12), 98 (hU6 variant 13), and 99 (hU6 variant 14)) all edited in the range of 15-20%, compared to 55% for construct AAV53. Other Pol III variants (constructs AAV94 (hU6 variant 9), 95 (hU6 variant 10) and 100 (hU6 variant 15)) all exhibited higher levels of editing at around 32% editing while construct 101 resulted in 48% editing. These promoters are all smaller than the Pol III promoter in the base construct AAV53, as shown in the scatterplot of FIG. 19, depicting transgene size of all AAV variants tested having engineered U6 RNA promoters on the X-axis vs. percent of mNPCs edited on the Y-axis.

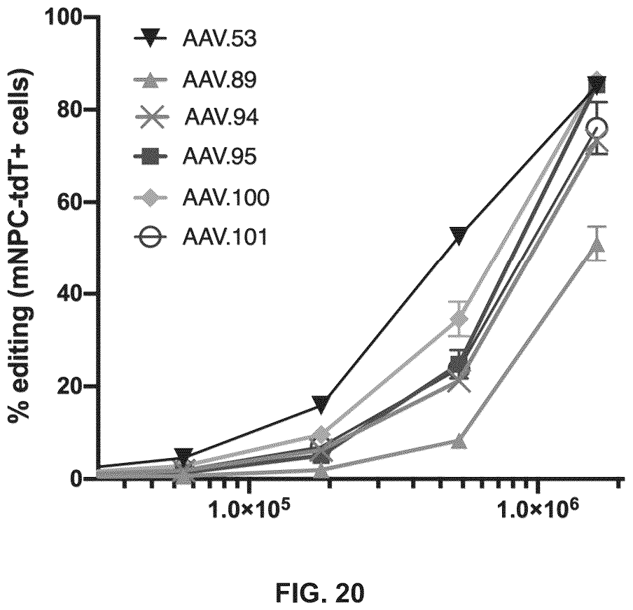

The results depicted in FIG. 20 show that these constructs with engineered U6 promoters with CasX 491, scaffold variant 174, and spacer 12.7, when delivered as AAV, were able to edit the target stop cassette in mNPCs in a dose-dependent fashion. Variable rates of editing mediated by AAV with constructs AAV.94, AAV.95, AAV.100, and AAV.101 were seen, all editing at rates between the base construct AAV.53 and AAV.89, which has the same Pol III promoter as AAV.85 from FIG. 16.

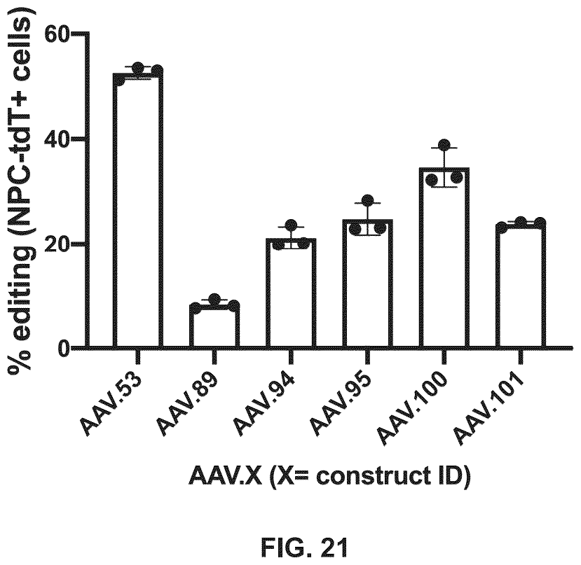

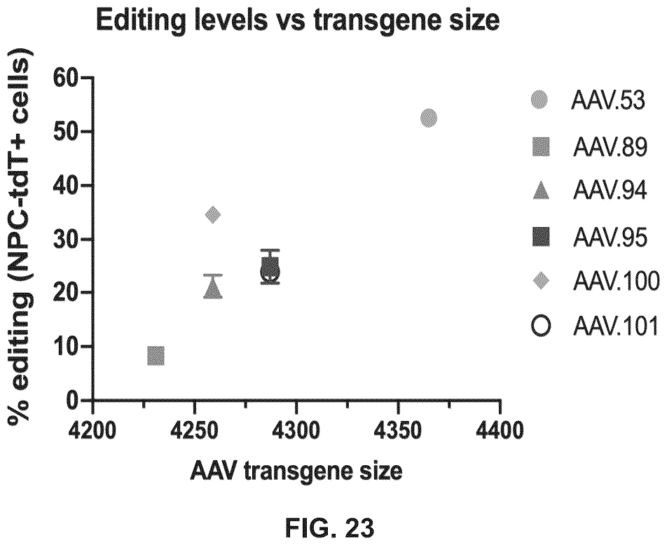

The results depicted in FIG. 21 show that constructs with engineered U6 promoters combined with CasX protein 491, scaffold variant 174 and spacer 12.7, when delivered as AAV, were able to edit the target stop cassette in mNPCs. Variable rates of editing with AAV with constructs AAV.94, AAV.95, AAV.100, and AAV.101 were seen, all editing at rates between the base construct AAV.53 and AAV.89, which has the same Pol III promoter as AAV.85 from FIG. 16. FIG. 23 shows the results as a scatterplot of editing versus transgene size.

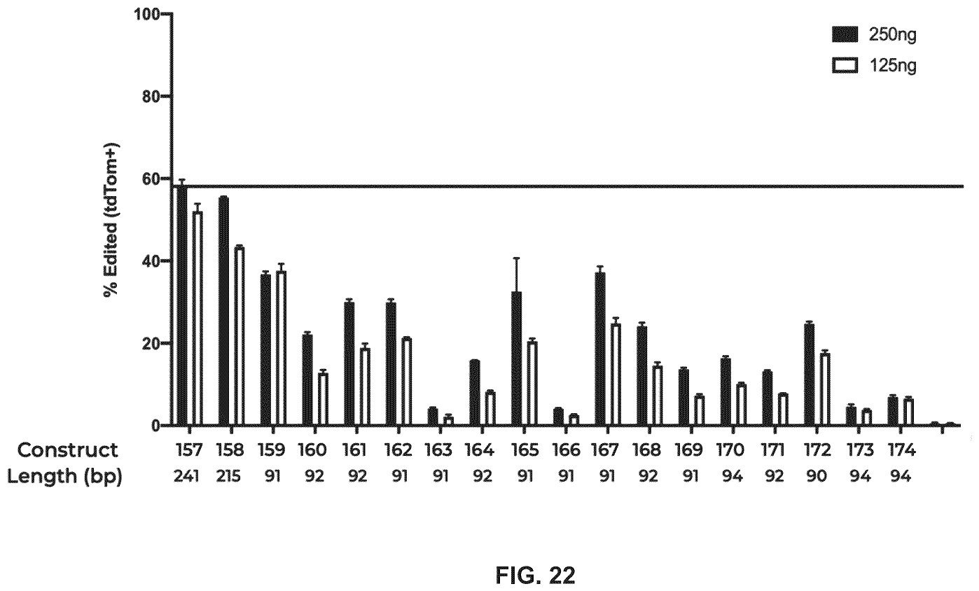

The results depicted in FIG. 22 demonstrate that AAV constructs with rationally engineered Pol III promoters, sequences encoding for CasX protein 491, and scaffold variant 174 with spacer 12.7, were able to edit the target tdTomato stop cassette at varying efficiencies when nucleofected as AAV transgene plasmids into mouse NPCs at doses 250 ng and 125 ng. Constructs 159 to 174 were designed to minimize the size of the promoter relative to the base U6 (construct ID 157) or H1 (construct ID 158) promoter, and constructs 160 to 174 were engineered as short, hybrid variants based on a core region of the H1 promoter (construct ID 159) with variations of domain swaps from 7SK and/or U6 promoters. The results of FIG. 22 show that most of these promoter variants, which are substantially shorter than the base U6 and H1 promoters, were able to function as Pol III promoters to drive sufficient gRNA transcription and editing at the tdTomato locus. Specifically, constructs 159, 161, 162, 165, and 167 were able to achieve at least 30% editing at the higher dose of 250 ng. These variants serve as promoter alternatives in AAV construct design that would permit significant reductions in AAV cargo capacity while driving adequate gRNA expression for targeted editing.

The results of these experiments demonstrate that expression of small CRISPR systems, such as CasX and gRNAs, can be modulated in various ways by utilizing alternative RNA promoters to express the gRNA. While most other CRISPR systems utilized in AAV do not have sufficient space in the transgene to include a separate promoter to express the gRNA, the CasX CRISPR system, and other systems with similarly small size, enable the use of multiple gRNA promoters of varying lengths within a single AAV transgene. These promoters can be used to differentially control expression and editing by the AAV transgene. The data also show that shorter versions of Pol III promoters can be engineered to retain their ability to facilitate transcription of functional guides. This increases the capacity of the AAV transgene to include additional promoters and/or accessory elements. Furthermore, adjusting other elements in this AAV transgene allows for the combination of multiple gRNA transcriptional units that could result in the following: 1) increased gRNA expression and thus CasX-mediated editing; or 2) driving the expression of more than one gRNA from a single AAV system, which would enable the ability to deliver CasX with a dual-gRNA system from a single AAV vector for targeted editing at different locations in the target nucleic acid (further discussed in Example 9).

### Example 6: Choice of Poly(A) Signal Enhances Potency of AAV Vectors

Experiments were conducted to demonstrate that small CRISPR proteins, such as CasX, can be expressed from an AAV genome utilizing a variety of polyadenylation (poly(A)) signals. Specifically, use of sequences encoding smaller CRISPR systems enable the inclusion of larger poly(A) signal sequences in the transgene of AAV vectors. In addition, experiments were conducted to demonstrate that the inclusion of shorter synthetic poly(A) signal sequences in the AAV constructs allows for further reductions in AAV transgene cargo capacity.

### Materials and Methods

AAV plasmid cloning: Poly(A) signal sequences were ordered as gene fragments and cloned into vector restriction sites according to standard techniques.

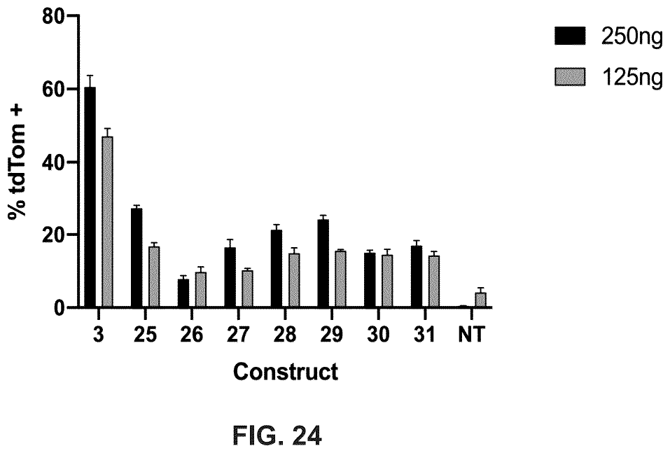

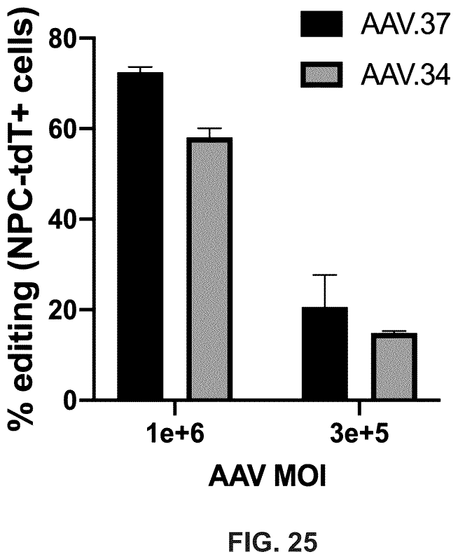

To generate the AAV plasmids assessed in the experiment data presented in FIG. 24 and FIG. 25, the methods of Example 1 were used for cloning and quality control of the constructs, as well as for plasmid nucleofection and FACS analysis. The sequences of the poly(A) signals are presented in Table 12. The sequences of the additional components of AAV constructs, with the exception of sequences encoding the CasX (Table 63) and the one or more gRNA (Table 26), are listed in Table 64.

TABLE 12
Poly(A) signal sequences

| AAV Construct | Poly(A) | Size | SEQ |
|---|---|---|---|
| ID | signal | (bp) | ID NO |
| 1, 3, 37, 232 | bGH | 208 | 514 |
| 24, 227 | hGH | 623 | 515 |
| 25, 228 | hGHshort | 477 | 516 |
| 26, 231 | HSVTK | 49 | 517 |
| 27, 221 | SynPolyA | 49 | 518 |
| 28, 226 | SV40 | 122 | 519 |
| 29 | SV40short | 82 | 520 |
| 30, 229 | bglob | 395 | 521 |
| 31, 230 | bglobshort | 56 | 522 |
| 34 | SV40polyA late (SL) | 181 | 523 |
| 222 | T7 Tphi | 119 | 3997 |
| 223 | CaMV | 175 | 3998 |
| 224 | RDH1 | 171 | 3999 |
| 225 | Sv40 polyA late | 241 | 4000 |

Methods for plasmid nucleofection and assessing activity by FACS were conducted as described in Example 1.

### Neuronal Cell Culture:

All neuronal cell culture was performed using N2B27-based media. To induce neuronal differentiation, iPSCs were plated in neuronal plating media (N2B27 base media with 1 μg/mL doxycycline, 200 μM L-ascorbic acid, 1 μM dibutyryl cAMP sodium salt, 10 μM CultureOne, 100 ng/ml of BDNF, 100 ng/ml of GDNF). iNs (induced neurons) were dissociated, aliquoted, and frozen for long term storage after three days of differentiation (DIV3). DIV3 iNs were thawed and seeded on a 96-well plate at ˜30,000-50,000 cells per well. iNs were cultured for one week in plating media and thereafter, half-media changes were performed once every week using feeding media (N2B27 base media with 200 μM L-ascorbic acid, 1 μM dibutyryl cAMP sodium salt, 200 ng/ml of BDNF, 200 ng/ml of GDNF).

### AAV Transduction of iNs In Vitro:

24 hours prior to transduction, ˜30,000-50,000 iNs per well were seeded on Matrigel-coated 96-well plates. AAVs expressing the CasX:gRNA system, which included constructs encoding for poly(A) signal sequences listed in Table 12, were then diluted in neuronal plating media and added to cells. Cells were transduced at two MOIs (1E2 or 1E3 vg/cell). Seven days post-transduction, iNs were replenished using feeding media. Seven days post-transduction, cells were lifted using lysis buffer, 4-well replicates were pooled per experimental condition, and genomic DNA (gDNA) was harvested and prepared for editing analysis at the B2M locus using next generation sequencing (NGS).

### NGS Processing and Analysis:

Genomic DNA (gDNA) from harvested cells were extracted using the Zymo Quick-DNA™ Miniprep Plus kit following the manufacturer's instructions. Target amplicons were formed by amplifying regions of interest from 200 ng of extracted gDNA with a set of primers specific to the target locus, such as the human B2M gene. These gene-specific primers contained an additional sequence at the 5′ end to introduce an Illumina™ adapter and a 16-nucleotide unique molecule identifier. Amplified DNA products were purified with the Ampure XP DNA cleanup kit. Quality and quantification of the amplicon were assessed using a Fragment Analyzer DNA Analysis kit (Agilent, dsDNA 35-1500 bp). Amplicons were sequenced on the Illumina™ Miseq™ according to the manufacturer's instructions. Raw fastq files from sequencing were quality-controlled and processed using cutadapt v2.1, flash2 v2.2.00, and CRISPResso2 v2.0.29. Each sequence was quantified for containing an insertion or deletion (indel) relative to the reference sequence, in a window around the 3′ end of the spacer (30 bp window centered at −3 bp from 3′ end of spacer). CasX activity was quantified as the total percent of reads that contain insertions, substitutions, and/or deletions anywhere within this window for each sample.

### Cloning Poly(A) Library for High-Throughput Screen:

To identify poly(A) signals that enable CasX to be expressed in an AAV genome in a high-throughput manner, a massively parallel reporter assay was conducted. Briefly, 10,000 poly(A) constructs (1,000 unique poly(A) signal sequences×10 barcodes per poly(A) signal sequence) were amplified, digested, and ligated into a restriction enzyme-digested AAV plasmid backbone harboring sequences coding for CasX protein 491 and gRNA scaffold variant 235 with spacer 7.37 (GGCCGAGAUGUCUCGCUCCG; SEQ ID NO: 2709) targeting the endogenous B2M (beta-2-microglobulin) locus. The poly(A) signal sequences are provided in SEQ ID NOS: 2991-3991 of the accompanying sequence listing. Cloned AAV plasmids were then transformed into electrocompetent bacterial cells (MegaX DH10B T1^{R} Electrocomp™). Titer of poly(A) signal sequence library transformation was determined by counting E. coli colony-forming units (CFUs) from electroporated library MEGA-X Competent cells. After transformation and overnight growth in liquid cultures, the library was purified using the ZymoPURE™ Midiprep Kit. To determine adequate library coverage, barcoded amplicons were detected via PCR amplification followed by next generation sequencing (NGS) on the Illumina™ MiSeq™. Raw fastq files were processed using cutadapt v3.5, mapped using bowtie2 v9.3.0, and barcodes were extracted using custom software. Barcoded counts were normalized by total read counts to calculate the representation of each library member.

### AAV Vector Production:

AAV vectors were produced according to standard methods, which are described in Example 1.

To determine the viral genome (vg) titer, 1 μL from crude lysate viruses was digested with DNase and Proteinase K, followed by quantitative PCR. 5 μL of digested virus was used in a 25 μL qPCR reaction composed of IDT primetime master mix and a set of primer and 6′FAM/Zen/IBFQ probe (IDT) designed to amplify a 62 bp-fragment located in the AAV2-ITR. Ten-fold serial dilutions of an AAV ITR plasmid was used as reference standards to calculate the titer (vg/mL) of viral samples.

After production, AAVs from the pooled library were lysed to release AAV virion DNA, which was then purified according to standard methods. Barcoded amplicons were PCR-amplified from the viral DNA input, sequenced, and processed as described earlier to determine the coverage of the AAV pool. Barcode counts were normalized by total read counts to calculate an RPM value.

### AAV Transduction and Method for RNA Transcript Analysis:

10,000 HEK293 Ts were seeded per well in PLF-coated 24-well plates 48 hours before AAV transduction. At time of transduction, HEK293 Ts were transduced with the pooled library of AAVs containing the library of poly(A) signal sequences. All viral infection conditions were performed in triplicate, with normalized number of vg among experimental vectors, at an MOI of 1E5 and 1E4 vg/cell. Two days post-transduction, total RNA was isolated and converted into cDNA by reverse transcription. Barcoded amplicons were PCR-amplified from the resulting cDNA, sequenced, and processed as described earlier. Barcode counts were normalized by total read counts to calculate an RPM value. To calculate the RNA abundance ratio for each poly(A) signal sequence from the library, normalized barcode counts from cDNA amplicons were divided by normalized barcode counts from viral DNA input. Poly(A) signal sequences with a high RNA abundance ratio, i.e., with the highest accumulation in HEK293 Ts, were identified as the poly(A) signal sequences of interest for further CasX editing assessments in vitro or in vivo.

### Results:

The results portrayed in FIG. 24 demonstrate that AAV constructs with several alternative poly(A) signals, in combination with CasX variant 491, scaffold variant 174 and spacer 12.7, when delivered by nucleofection of AAV transgene plasmid, were able to edit the target stop cassette in mNPCs at doses of 250 ng and 125 ng. Construct AAV3 (bGH poly(A)) showed the highest potency out of the three constructs tested in this experiment, editing the target locus at 60% efficiency (250 ng dose). Constructs 28 (SV40) and 29 (SV40 short), which have poly(A) signal sequences that are 59% and 39% of the size of the poly(A) signal sequence of construct 3, respectively (see Table 13), edited at 21% and 24% respectively (250 ng dose).

TABLE 13
Poly(A) construct variants

| AAV Construct | Poly(A) Signal Size | AAV Transgene Size |
|---|---|---|
| ID | (bp) | (bp) |
| 3 | 208 | 4550 |
| 25 | 477 | 4795 |
| 26 | 49 | 4367 |
| 27 | 49 | 4367 |
| 28 | 122 | 4440 |
| 29 | 82 | 4400 |
| 30 | 395 | 4713 |
| 31 | 56 | 4374 |
| 34 | 181 | 4565 |
| 37 | 208 | 4619 |

The results portrayed in FIG. 25 demonstrate that the two different poly(A) signals, combined with CasX protein 491, scaffold variant 174 and spacer 12.7, when delivered as an AAV vector, were able to edit the target stop cassette in mNPCs. AAV.34 and AAV.37 were generated with transgene constructs 34 (with a poly(A) signal of 186 nucleotides and a total transgene length of 4565 nucleotides) and 37 (with a poly(A) signal of 208 nucleotides and a total transgene length of 4619 nucleotides), respectively. Each vector displayed dose-dependent editing at the target locus, and AAV.34, which contains a shorter poly(A) signal, had approximately 75% of the editing potency of AAV.37 for both doses.

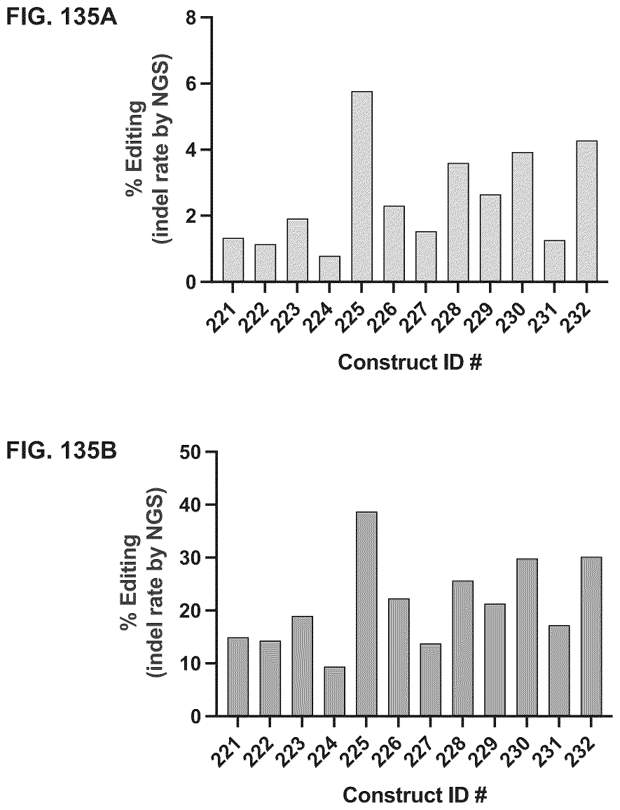

The results portrayed in FIGS. 135A-135B demonstrate that use of AAV constructs containing the SV40 poly(A) late poly(A) signal (construct ID 225) resulted in improved editing compared to that when using constructs with other poly(A) signals. Furthermore, multiple constructs containing poly(A) signals less than 70 bp contained high activity. Each vector displayed dose-dependent editing at the target locus.

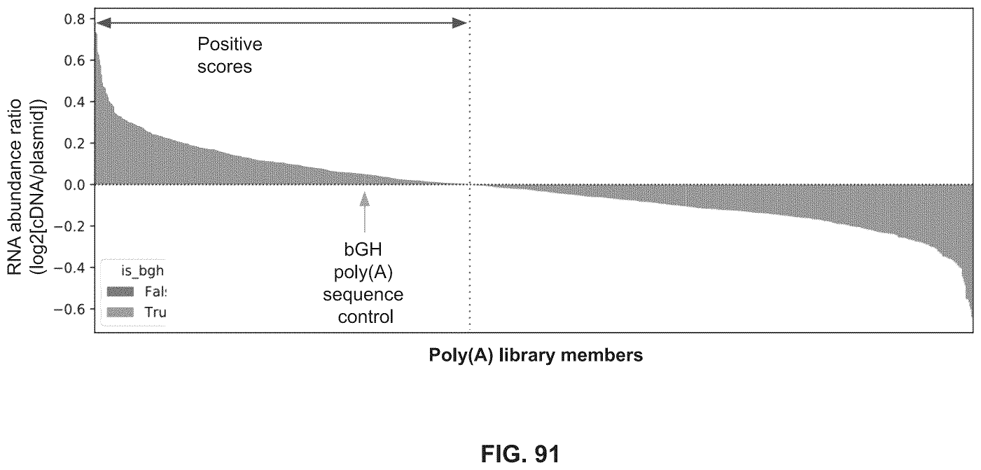

Experiments were performed in HEK293T cells to screen for poly(A) signal sequences for incorporation into future AAV construct designs that would improve CasX expression. As described above, poly(A) signal sequences with a high RNA abundance ratio would be identified as the poly(A) signal sequences of interest for further testing. The RNA abundance ratio was calculated across ten technical replicates by summing the counts across technical replicates and plotted for each unique poly(A) signal sequence from the library for each biological replicate (FIG. 91). Approximately 42% of poly(A) signal sequences screened demonstrated a positive RNA abundance ratio in any of the three biological replicates assessed, indicating that use of these poly(A) signal sequences resulted in higher CasX expression. Here, the bGH poly(A) signal sequence served as a positive control and is annotated in FIG. 91. The mean RNA abundance ratio was also calculated and plotted against the sequence length for each poly(A) signal candidate (data not shown). It was determined that approximately 71% of the poly(A) signal sequences with a positive RNA abundance ratio in any of the three biological replicates also have a sequence length shorter than the sequence of the bGH control (109 bp) from start of the sequence to polyadenylation site. A list of poly(A) signal sequences with a positive mean RNA abundance ratio across all three biological replicates and with a sequence length shorter than bGH across all three biological replicates is presented in SEQ ID NOS: 2710-2859 as set forth in Table 14. These identified poly(A) signal sequences, as well as sequences listed in Table 15, will be incorporated in future AAV construct designs for further assessment in vitro or in vivo. These findings support use of the unique poly(A) signal sequences in designing AAV vectors that would provide additional flexibility for increased AAV transgene cargo capacity while potentially enhancing CasX expression and editing efficiency.

Overall, the results demonstrate that the expression of small CRISPR proteins, such as CasX, can be modulated by poly(A) signals of varying lengths. Longer poly(A) signal sequences can be utilized in the siAAV constructs for enhanced CasX activity, while shorter poly(A) signal sequences can be utilized in the siAAV constructs to make more sequence space available for the inclusion of additional accessory elements within the siAAV transgene.

TABLE 14
List of poly(A) signals identified with a positive mean RNA abundance
ratio and sequence length shorter than bGH control (109 bp)

| Name | SEQ ID NO: | Poly(A) ID |
|---|---|---|
| SPATA24 | 2710 | Poly(A)_812 |
| C14orf28 | 2711 | Poly(A)_269 |
| TSKS | 2712 | Poly(A)_554 |
| NUP153 | 2713 | Poly(A)_833 |
| RPS12 | 2714 | Poly(A)_862 |
| IL17RC | 2715 | Poly(A)_709 |
| CALM2 | 2716 | Poly(A)_594 |
| KLK13 | 2717 | Poly(A)_562 |
| STPG4 | 2718 | Poly(A)_593 |
| RNF181 | 2719 | Poly(A)_612 |
| SPAG4 | 2720 | Poly(A)_663 |
| RHOT2 | 2721 | Poly(A)_306 |
| GLG1 | 2722 | Poly(A)_359 |
| ACTA2 | 2723 | Poly(A)_105 |
| SMPD2 | 2724 | Poly(A)_859 |
| MRPL52 | 2725 | Poly(A)_254 |
| ETV2 | 2726 | Poly(A)_499 |
| TMEM102 | 2727 | Poly(A)_383 |
| CLCNKB | 2728 | Poly(A)_13 |
| AAAS | 2729 | Poly(A)_203 |
| SPATA7 | 2730 | Poly(A)_273 |
| FKBP11 | 2731 | Poly(A)_200 |
| NOP16 | 2732 | Poly(A)_821 |
| CDC34 | 2733 | Poly(A)_450 |
| NLRX1 | 2734 | Poly(A)_188 |
| PRPF8 | 2735 | Poly(A)_370 |
| EIF3K | 2736 | Poly(A)_508 |
| KRTCAP2 | 2737 | Poly(A)_71 |
| RPS15 | 2738 | Poly(A)_455 |
| ELOC | 2739 | Poly(A)_911 |
| TFPT | 2740 | Poly(A)_568 |
| CEP350 | 2741 | Poly(A)_86 |
| RAB33A | 2742 | Poly(A)_993 |
| WDR36 | 2743 | Poly(A)_808 |
| ARMC12 | 2744 | Poly(A)_846 |
| NNMT | 2745 | Poly(A)_184 |
| NAA50 | 2746 | Poly(A)_744 |
| RPL14 | 2747 | Poly(A)_716 |
| RPL18 | 2748 | Poly(A)_537 |
| TMEM205 | 2749 | Poly(A)_474 |
| CCDC180 | 2750 | Poly(A)_946 |
| KCNJ13 | 2751 | Poly(A)_645 |
| SCO2 | 2752 | Poly(A)_707 |
| TUT1 | 2753 | Poly(A)_145 |
| PTH2 | 2754 | Poly(A)_543 |
| THOC1 | 2755 | Poly(A)_442 |
| ACKR1 | 2756 | Poly(A)_76 |
| RPL38 | 2757 | Poly(A)_429 |
| SAMD8 | 2758 | Poly(A)_102 |
| RPS8 | 2759 | Poly(A)_33 |
| SCNM1 | 2760 | Poly(A)_55 |
| HEXDC | 2761 | Poly(A)_441 |
| EXOSC4 | 2762 | Poly(A)_931 |
| IDH3B | 2763 | Poly(A)_652 |
| UBE2D2 | 2764 | Poly(A)_813 |
| CBR3 | 2765 | Poly(A)_678 |
| GPR19 | 2766 | Poly(A)_195 |
| ALKBH7 | 2767 | Poly(A)_462 |
| UBE2D3 | 2768 | Poly(A)_782 |
| SUPV3L1 | 2769 | Poly(A)_99 |
| SENP3 | 2770 | Poly(A)_384 |
| RNF166 | 2771 | Poly(A)_366 |
| EEF1D | 2772 | Poly(A)_927 |
| TEKT1 | 2773 | Poly(A)_380 |
| PSTK | 2774 | Poly(A)_115 |
| RETREG1 | 2775 | Poly(A)_792 |
| RPL7 | 2776 | Poly(A)_910 |
| SLIT2 | 2777 | Poly(A)_775 |
| CALHM6 | 2778 | Poly(A)_861 |
| C1orf53 | 2779 | Poly(A)_87 |
| AURKAIP1 | 2780 | Poly(A)_3 |
| EEF1D | 2781 | Poly(A)_928 |
| CRYGD | 2782 | Poly(A)_636 |
| MSRB3 | 2783 | Poly(A)_215 |
| ABHD1 | 2784 | Poly(A)_584 |
| HCCS | 2785 | Poly(A)_972 |
| POLD1 | 2786 | Poly(A)_558 |
| ZBTB48 | 2787 | Poly(A)_7 |
| UBC | 2788 | Poly(A)_233 |
| MRPL52 | 2789 | Poly(A)_253 |
| CORO7-PAM16 | 2790 | Poly(A)_321 |
| DPM3 | 2791 | Poly(A)_70 |
| RPS9 | 2792 | Poly(A)_570 |
| SYCE1L | 2793 | Poly(A)_362 |
| AKAP8L | 2794 | Poly(A)_479 |
| DHX30 | 2795 | Poly(A)_719 |
| RABGGTA | 2796 | Poly(A)_265 |
| RNF181 | 2797 | Poly(A)_611 |
| PPP1R35 | 2798 | Poly(A)_881 |
| RRP9 | 2799 | Poly(A)_735 |
| VPS29 | 2800 | Poly(A)_225 |
| NUP85 | 2801 | Poly(A)_431 |
| EXOC3L1 | 2802 | Poly(A)_347 |
| TAF10 | 2803 | Poly(A)_126 |
| PPP1R13L | 2804 | Poly(A)_531 |
| POLB | 2805 | Poly(A)_907 |
| INTS8 | 2806 | Poly(A)_916 |
| PLA2G1B | 2807 | Poly(A)_229 |
| ZFAND5 | 2808 | Poly(A)_945 |
| RPL30 | 2809 | Poly(A)_918 |
| SGF29 | 2810 | Poly(A)_329 |
| NUDT14 | 2811 | Poly(A)_279 |
| IFT122 | 2812 | Poly(A)_751 |
| CUTA | 2813 | Poly(A)_842 |
| FBL | 2814 | Poly(A)_516 |
| ZCRB1 | 2815 | Poly(A)_197 |
| C2orf70 | 2816 | Poly(A)_583 |
| CCDC33 | 2817 | Poly(A)_291 |
| MPHOSPH10 | 2818 | Poly(A)_603 |
| BAG6 | 2819 | Poly(A)_836 |
| C11orf80 | 2820 | Poly(A)_167 |
| PPIL6 | 2821 | Poly(A)_858 |
| SNRNP27 | 2822 | Poly(A)_600 |
| NUP93 | 2823 | Poly(A)_339 |
| C17orf98 | 2824 | Poly(A)_405 |
| ATRIP | 2825 | Poly(A)_721 |
| TRO | 2826 | Poly(A)_983 |
| U2AF1L4 | 2827 | Poly(A)_502 |
| PRIM1 | 2828 | Poly(A)_211 |
| S100A2 | 2829 | Poly(A)_65 |
| PRKAA1 | 2830 | Poly(A)_794 |
| CPVL | 2831 | Poly(A)_870 |
| PIP | 2832 | Poly(A)_896 |
| MAPKAPK5 | 2833 | Poly(A)_227 |
| IL13RA2 | 2834 | Poly(A)_988 |
| C2orf70 | 2835 | Poly(A)_582 |
| TMEM219 | 2836 | Poly(A)_331 |
| EXOSC8 | 2837 | Poly(A)_238 |
| CUTA | 2838 | Poly(A)_843 |
| PLCB2 | 2839 | Poly(A)_282 |
| PCGF1 | 2840 | Poly(A)_608 |
| PFDN5 | 2841 | Poly(A)_201 |
| IFT27 | 2842 | Poly(A)_697 |
| VMA21 | 2843 | Poly(A)_994 |
| PSMB1 | 2844 | Poly(A)_864 |
| EEF1D | 2845 | Poly(A)_929 |
| ARMC3 | 2846 | Poly(A)_98 |
| AP1M1 | 2847 | Poly(A)_480 |
| BOLA3 | 2848 | Poly(A)_604 |
| TMEM258 | 2849 | Poly(A)_143 |
| TCTEX1D2 | 2850 | Poly(A)_764 |
| ALAS1 | 2851 | Poly(A)_737 |
| AKAP14 | 2852 | Poly(A)_991 |
| TEX10 | 2853 | Poly(A)_947 |
| CTNNBL1 | 2854 | Poly(A)_665 |
| UBL5 | 2855 | Poly(A)_472 |
| FGF21 | 2856 | Poly(A)_540 |
| PPP2R3C | 2857 | Poly(A)_268 |
| SEPT11 | 2858 | Poly(A)_778 |
| DNLZ | 2859 | Poly(A)_963 |

TABLE 15
List of additional poly(A) signals for incorporation
into AAV construct designs

| Name | Poly(A) ID | SEQ ID NO: |
|---|---|---|
| RPL10 | Poly(A)_1000 | 4110 |
| RPL11 | Poly(A)_16 | 4111 |
| RPL12 | Poly(A)_952 | 4112 |
| RPL13A | Poly(A)_546 | 4113 |
| RPL22 | Poly(A)_6 | 4114 |
| RPL22L1 | Poly(A)_757 | 4115 |
| RPL26 | Poly(A)_390 | 4116 |
| RPL26 | Poly(A)_391 | 4117 |
| RPL26 | Poly(A)_392 | 4118 |
| RPL27A | Poly(A)_127 | 4119 |
| RPL3 | Poly(A)_700 | 4120 |
| RPL30 | Poly(A)_919 | 4121 |
| RPL32 | Poly(A)_713 | 4122 |
| RPL35A | Poly(A)_765 | 4123 |
| RPL35A | Poly(A)_766 | 4124 |
| RPL5 | Poly(A)_43 | 4125 |
| RPL8 | Poly(A)_935 | 4126 |
| RPS11 | Poly(A)_547 | 4127 |
| RPS15A | Poly(A)_326 | 4128 |
| RPS16 | Poly(A)_513 | 4129 |
| RPS16 | Poly(A)_514 | 4130 |
| RPS19 | Poly(A)_521 | 4131 |
| RPS3A | Poly(A)_786 | 4132 |
| RPS5 | Poly(A)_574 | 4133 |
| RPS7 | Poly(A)_578 | 4134 |

### Example 7: Positions of Regulatory Elements Modulate Small CRISPR Protein Potency in AAV Vectors

Orientation (forward or reverse) and position (upstream or downstream of CRISPR gene) of regulatory elements such as the gRNA promoter and guide scaffold complex can modulate the underlying expression of the small CRISPR protein and the overall editing efficiency of CRISPR systems in AAV vectors. Experiments were performed to assess the best orientation and position of regulatory elements within the AAV genome to enhance the potency of small CRISPR proteins and guide RNAs.

### Materials and Methods:

AAV vector production and QC, nucleofection, AAV viral production and editing level assessment in mNPTC-tdT cells by FACS were conducted as described in Example 1.

### Results:

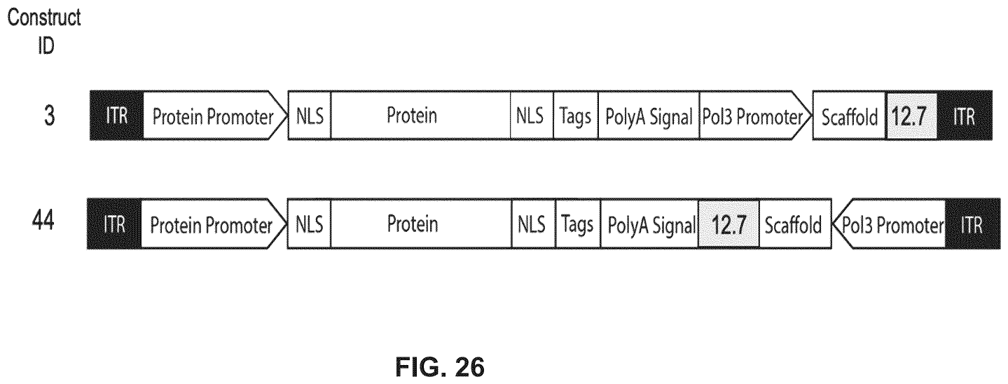

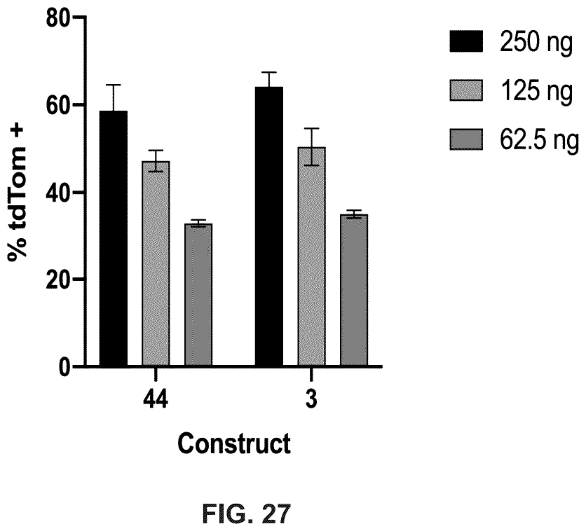

Construct AAV44 (configuration shown in FIG. 26, second from top) contains a Pol III promoter driving expression of guide scaffold 174 and spacer 12.7 in the reverse orientation of construct AAV.3 (top configuration in FIG. 26). The results depicted in FIG. 27 demonstrate that construct AAV44, when delivered by nucleofection of an AAV transgene plasmid, modifies the target stop cassette in mNPCs similarly to construct AAV3 at in a dose-dependent manner.

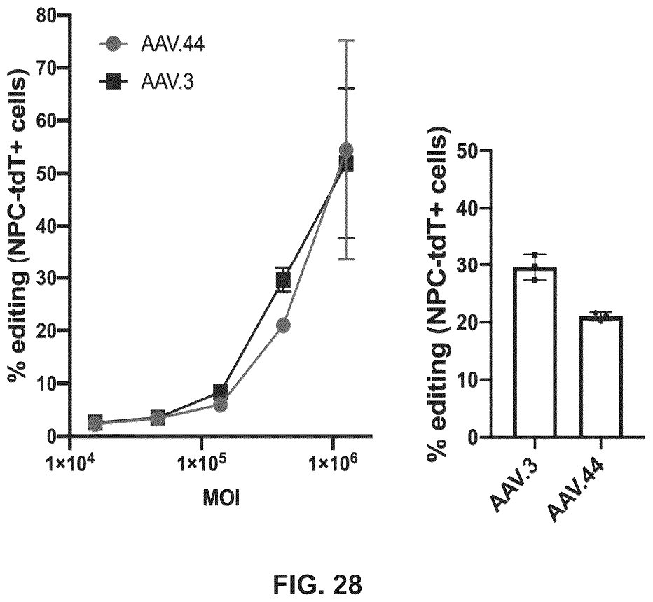

The results depicted in FIG. 28 show that construct AAV44, when delivered as an AAV vector, edits the target stop cassette in mNPCs, further supporting the utility of this construct. AAV.3 and AAV.44 were generated with transgene constructs AAV3 and AAV44, respectively. Each vector displayed dose-dependent editing at the target locus (FIG. 28, left panel, in which the vector was assayed using 3-fold dilutions). FIG. 28, right panel, shows editing results at an MOI of 3×10⁵, in which AAV.44 had 60% of the editing potency of the original configuration of vector AAV.3.

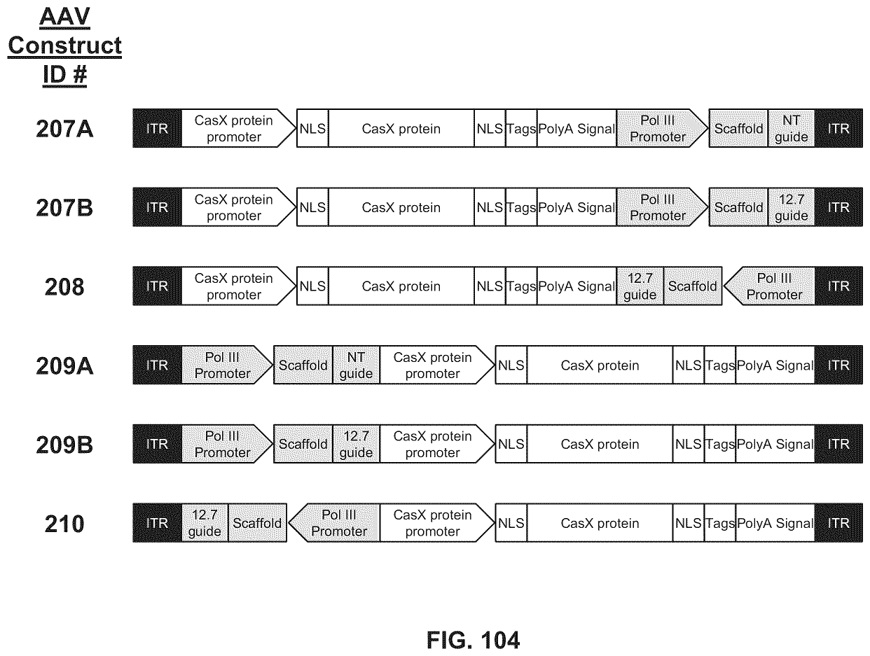

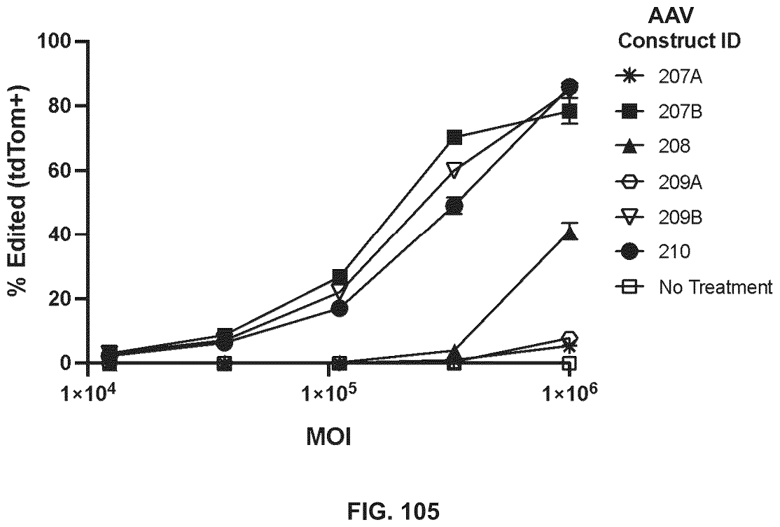

Additional configurations were explored, such that the gRNA transcriptional unit (Pol III U6 promoter driving the expression of the gRNA scaffold and indicated spacer) was placed either upstream or downstream of the CasX gene and was either in the forward or reverse orientation (FIG. 104). Table 16 lists the sequences of key AAV elements with varying positions and orientations of the gRNA promoter to drive gRNA expression (full AAV transgene sequences within ITRs are in Table 17. The resulting AAV constructs were used to produce AAVs, which were used to transduce mNPCs to assess editing level at the tdTomato locus. The results of this experiment are illustrated in FIG. 105. The data demonstrate that AAVs produced from Constructs 207B, 209B, and 210 were able to induce similar levels of editing at the tdTomato locus in a dose-dependent manner. Meanwhile, the configuration used in Construct 208, where the U6-sgRNA transcriptional unit was in the reverse orientation downstream of the CasX gene, appeared to adversely affect gene editing rate at the target locus.

TABLE 16
Sequences of key AAV elements with varying positions and orientations of the
gRNA transcriptional unit.

| AAV | Key Component | DNA | SEQ ID |
|---|---|---|---|
| Construct ID | Name | Sequence | NO: |
| 207A/B, 208, | 5′ ITR | CCTGCAGGCAGCTGCGCGCTCGCTCGCTCACTGAGGCC | 423 |
| 209A/B, 210 | | GCCCGGGCGTCGGGCGACCTTTGGTCGCCCGGCCTCAG | |
| | | TGAGCGAGCGAGCGCGCAGAGAGGGAGTGGCCAACTCC | |
| | | ATCACTAGGGGTTCCT | |
| | UbC promoter | GGCCTCCGCGCCGGGTTTTGGCGCCTCCCGCGGGCGCC | 464 |
| | | CCCCTCCTCACGGCGAGCGCTGCCACGTCAGACGAAGG | |
| | | GCGCAGCGAGCGTCCTGATCCTTCCGCCCGGACGCTCA | |
| | | GGACAGCGGCCCGCTGCTCATAAGACTCGGCCTTAGAA | |
| | | CCCCAGTATCAGCAGAAGGACATTTTAGGACGGGACTT | |
| | | GGGTGACTCTAGGGCACTGGTTTTCTTTCCAGAGAGCG | |
| | | GAACAGGCGAGGAAAAGTAGTCCCTTCTCGGCGATTCT | |
| | | GCGGAGGGATCTCCGTGGGGCGGTGAACGCCGATGATT | |
| | | ATATAAGGACGCGCCGGGTGTGGCACAGCTAGTTCCGT | |
| | | CGCAGCCGGGATTTGGGTCGCGGTTCTTGTTTGTGGAT | |
| | | CGCTGTGATCGTCACTTGGT | |
| | 5′ c-MYC NLS | CCAGCGGCCAAACGGGTGAAGCTGGAC | 838 |
| | CasX 491 | CAAGAGATCAAGAGAATCAACAAGATCAGAAGGAGACT | 791 |
| | | GGTCAAGGACAGCAACACAAAGAAGGCCGGCAAGACAG | |
| | | GCCCCATGAAAACCCTGCTCGTCAGAGTGATGACCCCT | |
| | | GACCTGAGAGAGCGGCTGGAAAACCTGAGAAAGAAGCC | |
| | | CGAGAACATCCCTCAGCCTATCAGCAACACCAGCAGGG | |
| | | CCAACCTGAACAAGCTGCTGACCGACTACACCGAGATG | |
| | | AAGAAAGCCATCCTGCACGTGTACTGGGAAGAGTTCCA | |
| | | GAAAGACCCCGTGGGCCTGATGAGCAGAGTTGCTCAGC | |
| | | CTGCCAGCAAGAAGATCGACCAGAACAAGCTGAAGCCC | |
| | | GAGATGGACGAGAAGGGCAATCTGACCACAGCCGGCTT | |
| | | TGCCTGCTCTCAGTGTGGCCAGCCTCTGTTCGTGTACA | |
| | | AGCTGGAACAGGTGTCCGAGAAAGGCAAGGCCTACACC | |
| | | AACTACTTCGGCAGATGTAACGTGGCCGAGCACGAGAA | |
| | | GCTGATTCTGCTGGCCCAGCTGAAACCTGAGAAGGACT | |
| | | CTGATGAGGCCGTGACCTACAGCCTGGGCAAGTTTGGA | |
| | | CAGAGAGCCCTGGACTTCTACAGCATCCACGTGACCAA | |
| | | AGAAAGCACACACCCCGTGAAGCCCCTGGCTCAGATCG | |
| | | CCGGCAATAGATACGCCTCTGGACCTGTGGGCAAAGCC | |
| | | CTGTCCGATGCCTGCATGGGAACAATCGCCAGCTTCCT | |
| | | GAGCAAGTACCAGGACATCATCATCGAGCACCAGAAGG | |
| | | TGGTCAAGGGCAACCAGAAGAGACTGGAAAGCCTGAGG | |
| | | GAGCTGGCCGGCAAAGAGAACCTGGAATACCCCAGCGT | |
| | | GACCCTGCCTCCTCAGCCTCACACAAAAGAAGGCGTGG | |
| | | ACGCCTACAACGAAGTGATCGCCAGAGTGAGAATGTGG | |
| | | GTCAACCTGAACCTGTGGCAGAAGCTGAAACTGTCCAG | |
| | | GGACGACGCCAAGCCTCTGCTGAGACTGAAGGGCTTCC | |
| | | CTAGCTTCCCTCTGGTGGAAAGACAGGCCAATGAAGTG | |
| | | GATTGGTGGGACATGGTCTGCAACGTGAAGAAGCTGAT | |
| | | CAACGAGAAGAAAGAGGATGGCAAGGTTTTCTGGCAGA | |
| | | ACCTGGCCGGCTACAAGAGACAAGAAGCCCTGAGGCCT | |
| | | TACCTGAGCAGCGAAGAGGACCGGAAGAAGGGCAAGAA | |
| | | GTTCGCCAGATACCAGCTGGGCGACCTGCTGCTGCACC | |
| | | TGGAAAAGAAGCACGGCGAGGACTGGGGCAAAGTGTAC | |
| | | GATGAGGCCTGGGAGAGAATCGACAAGAAGGTGGAAGG | |
| | | CCTGAGCAAGCACATTAAGCTGGAAGAGGAAAGAAGGA | |
| | | GCGAGGACGCCCAATCTAAAGCCGCTCTGACCGATTGG | |
| | | CTGAGAGCCAAGGCCAGCTTTGTGATCGAGGGCCTGAA | |
| | | AGAGGCCGACAAGGACGAGTTCTGCAGATGCGAGCTGA | |
| | | AGCTGCAGAAGTGGTACGGCGATCTGAGAGGCAAGCCC | |
| | | TTCGCCATTGAGGCCGAGAACAGCATCCTGGACATCAG | |
| | | CGGCTTCAGCAAGCAGTACAACTGCGCCTTCATTTGGC | |
| | | AGAAAGACGGCGTCAAGAAACTGAACCTGTACCTGATC | |
| | | ATCAATTACTTCAAAGGCGGCAAGCTGCGGTTCAAGAA | |
| | | GATCAAACCCGAGGCCTTCGAGGCTAACAGATTCTACA | |
| | | CCGTGATCAACAAAAAGTCCGGCGAGATCGTGCCCATG | |
| | | GAAGTGAACTTCAACTTCGACGACCCCAACCTGATTAT | |
| | | CCTGCCTCTGGCCTTCGGCAAGAGACAGGGCAGAGAGT | |
| | | TCATCTGGAACGATCTGCTGAGCCTGGAAACCGGCTCT | |
| | | CTGAAGCTGGCCAATGGCAGAGTGATCGAGAAAACCCT | |
| | | GTACAACAGGAGAACCAGACAGGACGAGCCTGCTCTGT | |
| | | TTGTGGCCCTGACCTTCGAGAGAAGAGAGGTGCTGGAC | |
| | | AGCAGCAACATCAAGCCCATGAACCTGATCGGCGTGGA | |
| | | CCGGGGCGAGAATATCCCTGCTGTGATCGCCCTGACAG | |
| | | ACCCTGAAGGATGCCCACTGAGCAGATTCAAGGACTCC | |
| | | CTGGGCAACCCTACACACATCCTGAGAATCGGCGAGAG | |
| | | CTACAAAGAGAAGCAGAGGACAATCCAGGCCAAGAAAG | |
| | | AGGTGGAACAGCGCAGAGCCGGCGGATACTCTAGGAAG | |
| | | TACGCCAGCAAGGCCAAGAATCTGGCCGACGACATGGT | |
| | | CCGAAACACCGCCAGAGATCTGCTGTACTACGCCGTGA | |
| | | CACAGGACGCCATGCTGATCTTCGAGAATCTGAGCAGA | |
| | | GGCTTCGGCCGGCAGGGCAAGAGAACCTTTATGGCCGA | |
| | | GAGGCAGTACACCAGAATGGAAGATTGGCTCACAGCTA | |
| | | AACTGGCCTACGAGGGACTGAGCAAGACCTACCTGTCC | |
| | | AAAACACTGGCCCAGTATACCTCCAAGACCTGCAGCAA | |
| | | TTGCGGCTTCACCATCACCAGCGCCGACTACGACAGAG | |
| | | TGCTGGAAAAGCTCAAGAAAACCGCCACCGGCTGGATG | |
| | | ACCACCATCAACGGCAAAGAGCTGAAGGTTGAGGGCCA | |
| | | GATCACCTACTACAACAGGTACAAGAGGCAGAACGTCG | |
| | | TGAAGGATCTGAGCGTGGAACTGGACAGACTGAGCGAA | |
| | | GAGAGCGTGAACAACGACATCAGCAGCTGGACAAAGGG | |
| | | CAGATCAGGCGAGGCTCTGAGCCTGCTGAAGAAGAGGT | |
| | | TTAGCCACAGACCTGTGCAAGAGAAGTTCGTGTGCCTG | |
| | | AACTGCGGCTTCGAGACACACGCCGATGAACAGGCTGC | |
| | | CCTGAACATTGCCAGAAGCTGGCTGTTCCTGAGAAGCC | |
| | | AAGAGTACAAGAAGTACCAGACCAACAAGACCACCGGC | |
| | | AACACCGACAAGAGGGCCTTTGTGGAAACCTGGCAGAG | |
| | | CTTCTACAGAAAAAAGCTGAAAGAAGTCTGGAAGCCCG | |
| | | CCGTG | |
| | 3′ c-MYC NLS | CCCGCCGCGAAGCGAGTGAAACTGGAC | 839 |
| | bGH poly(A) | CTGTGCCTTCTAGTTGCCAGCCATCTGTTGTTTGCCCC | 514 |
| | signal | TCCCCCGTGCCTTCCTTGACCCTGGAAGGTGCCACTCC | |
| | | CACTGTCCTTTCCTAATAAAATGAGGAAATTGCATCGC | |
| | | ATTGTCTGAGTAGGTGTCATTCTATTCTGGGGGGTGGG | |
| | | GTGGGGCAGGACAGCAAGGGGGAGGATTGGGAAGAGAA | |
| | | TAGCAGGCATGCTGGGGA | |
| 207A/B, | U6 promoter | GAGGGCCTATTTCCCATGATTCCTTCATATTTGCATAT | 661 |
| 209A/B | (Fwd) | ACGATACAAGGCTGTTAGAGAGATAATTGGAATTAATT | |
| | | TGACTGTAAACACAAAGATATTAGTACAAAATACGTGA | |
| | | CGTAGAAAGTAATAATTTCTTGGGTAGTTTGCAGTTTT | |
| | | AAAATTATGTTTTAAAATGGACTATCATATGCTTACCG | |
| | | TAACTTGAAAGTATTTCGATTTCTTGGCTTTATATATC | |
| | | TTGTGGAAAGGAC | |
| | Scaffold 235 | ACTGGCGCTTCTATCTGATTACTCTGAGCGCCATCACC | 698 |
| | (Fwd) | AGCGACTATGTCGTAGTGGGTAAAGCCGCTTACGGACT | |
| | | TCGGTCCGTAAGAGGCATCAGAG | |
| 207B, 209B | Spacer 12.7 | CTGCATTCTAGTTGTGGTTT | 462 |
| | (Fwd) | | |
| 207A, 209A | Spacer NT (Fwd) | GGGTCTTCGAGAAGACCC | 537 |
| 208, 210 | U6 promoter | GTCCTTTCCACAAGATATATAAAGCCAAGAAATCGAAA | 4001 |
| | (Rev Comp) | TACTTTCAAGTTACGGTAAGCATATGATAGTCCATTTT | |
| | | AAAACATAATTTTAAAACTGCAAACTACCCAAGAAATT | |
| | | ATTACTTTCTACGTCACGTATTTTGTACTAATATCTTT | |
| | | GTGTTTACAGTCAAATTAATTCCAATTATCTCTCTAAC | |
| | | AGCCTTGTATCGTATATGCAAATATGAAGGAATCATGG | |
| | | GAAATAGGCCCTC | |
| | Scaffold 235 | CTCTGATGCCTCTTACGGACCGAAGTCCGTAAGCGGCT | 4002 |
| | (Rev Comp) | TTACCCACTACGACATAGTCGCTGGTGATGGCGCTCAG | |
| | | AGTAATCAGATAGAAGCGCCAGT | |
| | Spacer 12.7 | AAACCACAACTAGAATGCAG | 4003 |
| | (Rev Comp) | | |
| | 3′ ITR | AGGAACCCCTAGTGATGGAGTTGGCCACTCCCTCTCTG | 424 |
| | | CGCGCTCGCTCGCTCACTGAGGCCGGGCGACCAAAGGT | |
| | | CGCCCGACGCCCGGGCTTTGCCCGGGCGGCCTCAGTGA | |
| | | GCGAGCGAGCGCGCAGCTGCCTGCAGG | |

TABLE 17
Sequences of AAV constructs within the AAV ITRs.

| Construct ID | SEQUENCE WITHIN AAV ITR (SEQ ID NO) |
|---|---|
| 207A | 4135 |
| 207B | 4136 |
| 208 | 4137 |
| 209A | 4138 |
| 209B | 4139 |
| 210 | 4140 |

The results of these experiments demonstrate that the orientation of parts within the AAV genome can be varied, yet result in sufficient expression of the CRISPR proteins and the guide RNA. This shows that specific orientations or positions of the regulatory elements relative to the encoded protein or RNA components may allow controlled modulation of expression in CasX-packaging siAAV constructs that contain one or multiple guides.

### Example 8: Small CRISPR Protein Potency is Enhanced by Inclusion of Additional Regulatory Elements in the AAV Vector that are not Possible a Larger Protein

These experiments demonstrated that transcriptional levels mediated by AAV vectors delivering small CRISPR proteins (such as CasX) can be enhanced by inclusion of different regulatory elements (intronic sequences, enhancers, etc.) that do not fit in AAV vectors expressing large transgene (e.g., spCas9) plasmids.

### Materials and Methods

Cloning and QC: A 4-part Golden Gate Assembly consisting of a pre-digested AAV backbone, small CRISPR protein-encoding DNA, and flanking 5′ and 3′ DNA sequences were used to generate AAV-cis plasmid as described in Example 1. 5′ sequences contained enhancer, protein promoter and N-terminal NLS, while 3′ sequences contained C-terminal NLS, WPRE, poly(A) signal, RNA promoter and guide RNA containing spacer 12.7. 5′ and 3′ parts were ordered as gene fragments, PCR-amplified, and assembled and assembled into AAV vectors. Cloning and plasmid QC, nucleofection, and FACS methods were conducted as described in Example 1.

Enhancement of editing by the inclusion of post-translation regulatory elements (PTRE) 1, 2, or 3 in the AAV cis plasmid 3 was tested in combination with different promoters driving expression of CasX. A first set of promoters were tested: transgene plasmids 4, 35, 36 37; transgene plasmids 5, 38, 39, 40 and transgene plasmids 6, 42, 43 have CasX protein expression driven by the CMV, UbC, EFS, CMV-s promoters, respectively. A second set of constructs tested included PTREs between the protein and poly(A) signal sequences and were generated with the Jet and JetUsp promoters compared to UbC promoter (transgenes 58, 72, 73, 74; transgenes 59, 75, 76, 77 and transgenes 53, 80 and 81, respectively) driving expression of CasX. PTRE sequences are listed in Table 18, and enhancer plus promoter sequences are listed in Table 19. The sequences of the additional components of AAV constructs, with the exception of sequences encoding the CasX (Table 63) and the one or more gRNA (Table 26), are listed in Table 64.

TABLE 18
Construct sequences of post-transcription
elements (PTRE) tested on base constructs

| Construct | PTRE | Size (bp) | SEQ ID NO |
|---|---|---|---|
| AAV35, AAV38, AAV72, AAV75 | 1 | 598 | 524 |
| AAV36, AAV39, AAV42, AAV73, | 2 | 593 | 525 |
| AAV76, AAV80 | | | |
| AAV37, AAV40, AAV43, AAV74, | 3 | 247 | 526 |
| AAV77, AAV81 | | | |

TABLE 19
Enhancer elements and sequences tested in
combination with the CMV core promoter

| Construct | Enhancer | Core promoter | Size (bp) | SEQ ID NO |
|---|---|---|---|---|
| AAV3 | CMV | CMV | 584 | 527 |
| AAV64 | N/A | CMV | 204 | 528 |
| AAV65 | Syn 1 | CMV | 414 | 529 |
| AAV66 | NPC5 | CMV | 314 | 530 |
| AAV67 | NPC7 | CMV | 324 | 531 |
| AAV68 | NPC127 | CMV | 304 | 532 |
| AAV69 | NPC190 | CMV | 364 | 533 |
| AAV70 | NPC249 | CMV | 274 | 534 |
| AAV71 | NPC286 | CMV | 354 | 535 |

### Results:

The effects of PTREs on transgene expression were assessed by cloning 3 enhancer sequences (PTRE1, PTRE2, and PTR3, Table 18) into an AAV-cis plasmid (construct AAV3) and construct plasmids containing shorter protein promoters (constructs AAV4-6, AAV53, AAV57 and AAV58 contain 400, 234, 335, 400, 164 and 326 bp promoter sequences, respectively).

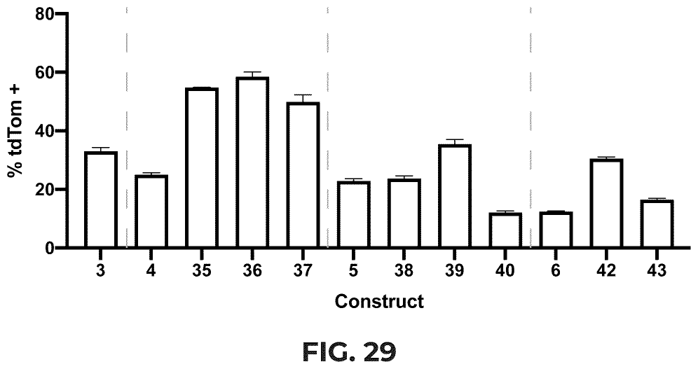

AAV-cis plasmid activity was first confirmed by nucleofection in mNPC-tdT cells. For each vector, addition of PTRE enhanced editing activity at various levels (FIG. 29). Table 20 provides the lengths of promoter and PTREs. The addition of PTRE2 to the transgene cassette showed the highest CasX editing activity enhancement, with a 2-fold increase in editing levels for construct AAV36 compared to construct AAV4 (58.5% vs 25%), a 1.5-fold increase for construct AAV39 (35.4% vs 22.9%) compared to construct AAV5 and a 3-fold increase for construct AAV42 compared to construct AAV6 (30.5% vs 12%). The shortest enhancer sequence, PTRE3, also increased protein activity at various levels among construct AAV37 and AAV43 compared to other vectors.

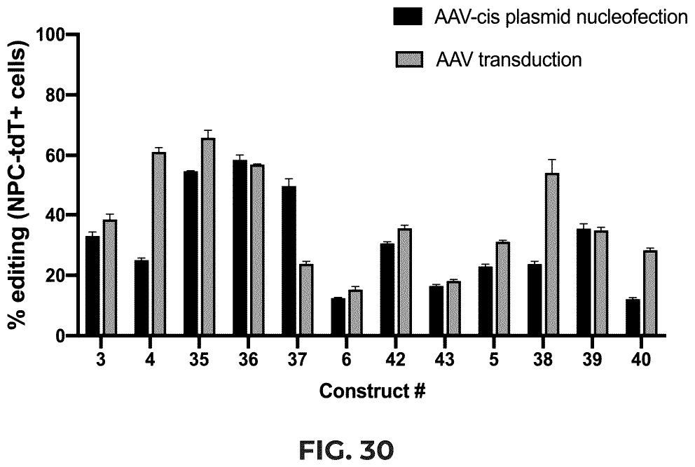

Improvements in editing levels were also observed when constructs were packaged into AAV. Inclusion of PTRE2 in transgene increased editing across the AAV vectors in a similar manner. Trends in on-target editing observed in mNPCs with the AAV infection generally correlated with the AAV plasmid nucleofection data set (FIG. 30).

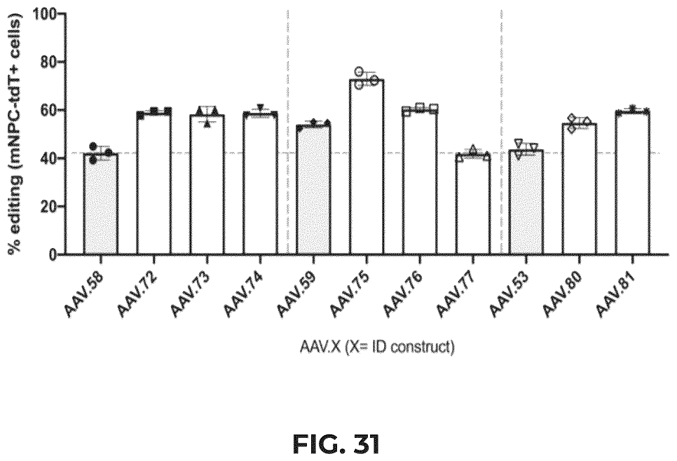

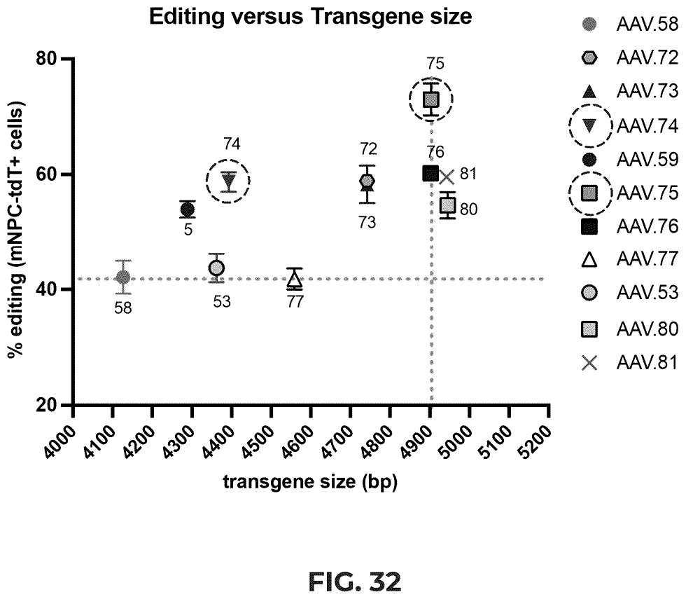

The trend was confirmed by testing another set of promoters with inclusion of these enhancer sequences. Across all AAV vectors tested, constructs including PTRE1 or PTRE2 in genomes yielded an average 1.5-fold increase compared to base vectors (FIG. 31). Unique combinations of short promoter and these post-transcriptional sequences led to the identification of vectors with increased editing levels with shorter promoters (e.g., AAV.74), which represents an advantage both for manufacturing siAAV under the carrying capacity limit of AAV, and for inclusion of more regulatory elements and CRISPR elements e.g., additional guides. Comparisons of editing versus transgene size are plotted in FIG. 32.

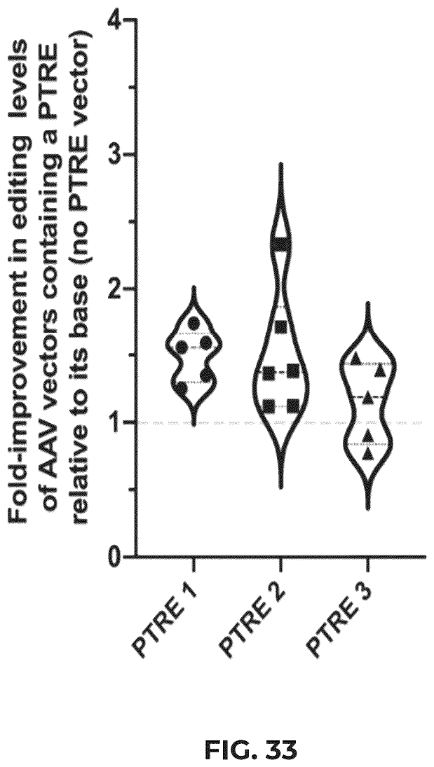

The results also demonstrate that inclusion of PTRE1 in the transgene plasmid improved editing levels across all promoters evaluated (FIG. 33), with less variability, while PTRE2 yielded the highest transgene improvement but with more variability across the promoters tested.

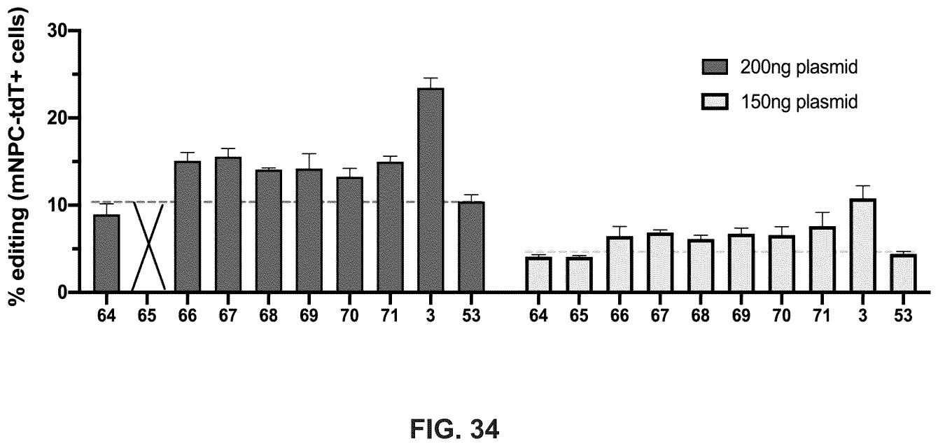

Several constructs with tissue-specific neuronal enhancers upstream of a single constitutive promoter were also tested. In this assay, 7 neuronal enhancer sequences (constructs AAV.65-72, sequences provided in Table 64) were cloned into a single AAV-cis plasmid (64) harboring a core CMV promoter and all demonstrated improved editing via nucleofection over base construct AAV.64 (FIG. 34). These constructs also outperformed construct AAV53, which contains a UbC promoter but did not outperform construct AAV3 which harbors the full CMV promoter (CMV enhancer+CMV core promoter).

TABLE 20
Constructs with or without PTREs and indicated sequence lengths
AAV Construct
(Sequence length indicated below)

| | 3 | 4 | 35 | 36 | 37 | 5 | 38 | 39 | 40 | 6 | 42 | 43 |
|---|---|---|---|---|---|---|---|---|---|---|---|---|
| Promoter | 584 | 400 | | | | 234 | | | | 335 | | |
| Length | | | | | | | | | | | | |
| PTRE 1 | — | — | 592 | — | — | — | 592 | — | — | — | — | — |
| PTRE 2 | — | — | — | 593 | — | — | — | 593 | — | — | 593 | — |
| PTRE 3 | — | — | — | — | 247 | — | — | — | 247 | — | — | 247 |
| AAV | 4550 | 4349 | 4964 | 4965 | 4619 | 4183 | 4798 | 4799 | 4453 | 4284 | 4900 | 4554 |
| transgene | | | | | | | | | | | | |

The results demonstrate that use of small promoters in the AAV transgene constructs permits the inclusion of additional accessory elements. These additional accessory elements, such as post-transcriptional regulatory elements to AAV-transgenes expressing CasX under the control of short but strong promoter sequences enable increased CasX expression and on-target editing while reducing cargo size, such that all components can be incorporated into a single siAAV vector.

### Example 9: Demonstration that a CasX:Dual-gRNA System Expressed from a Single AAV Vector can Edit the Target In Vitro

Experiments were performed to demonstrate the following: 1) Constructs of CasX and dual gRNAs expressed from an all-in-one AAV vector can edit the target locus; 2) the ability to package and deliver CasX with a dual-guide system within a single AAV vector for targeted editing; and 3) editing of a therapeutically-relevant locus by CasX and dual gRNAs delivered via a single AAV vector can excise the targeted genomic region. For the editing at a therapeutically-relevant locus by the CasX-dual-gRNA system, experiments were conducted to demonstrate the ability of CasX and the dual-guide system to mediate excision of a CTG repeat in the 3′UTR region of the human DMPK gene when delivered via AAVs in vitro into HEK293T cells. The ability to demonstrate editing mediated by the CasX:dual-gRNA system delivered and expressed from a single all-in-one AAV vector is significant because this is not achievable with traditionally-used Cas9-based systems.

### Materials and Methods

AAV plasmid cloning and nucleofection were conducted as described in Example 1.

Various configurations of two gRNA transcriptional unit blocks, also referred as “guide RNA stacks”, of the AAV transgene are illustrated in FIGS. 35-36 and FIG. 112.

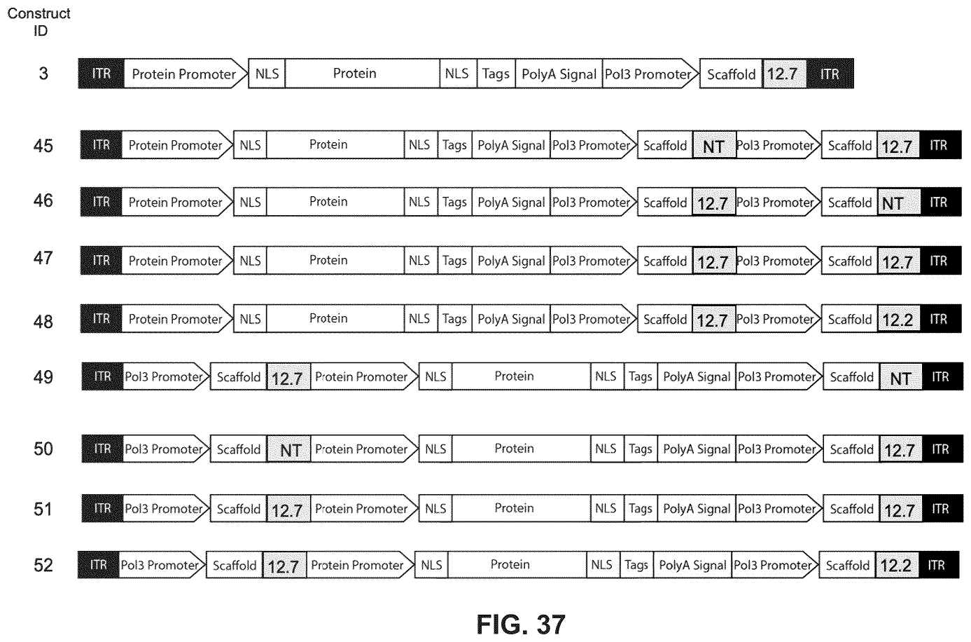

FIG. 37 illustrates the configurations of the dual-guide stacks, with each stack composed of a gRNA scaffold-spacer combination 174.12.7, 174.12.2 or 174.NT driven by the human U6 promoter (Table 11). These specific dual-guide stacks were investigated by cloning two gRNA stacks in a tail-to-tail orientation (Construct ID 45-49) on the 3′ end of the poly(A) or in the same transcriptional orientation as the protein promoter-CasX unit, one on each side of the CasX unit (Construct ID 50-52). Pentagon-shaped boxes for CasX protein promoter and Pol III gRNA promoter depict orientation of transcription (tapered point; 5′ to 3′ or 3′ to 5′ orientation). Spacer sequences are 12.2 (TATAGCATACATTATACGAA, SEQ ID NO: 536); 12.7 (CTGCATTCTAGTTGTGGTTT, SEQ ID NO: 462); and NT (GGGTCTTCGAGAAGACCC, SEQ ID NO: 537).

AAV vector production and titering were conducted as described in Example 1. AAV transduction and editing assessment via FACs sorting were conducted as described in Example 1.

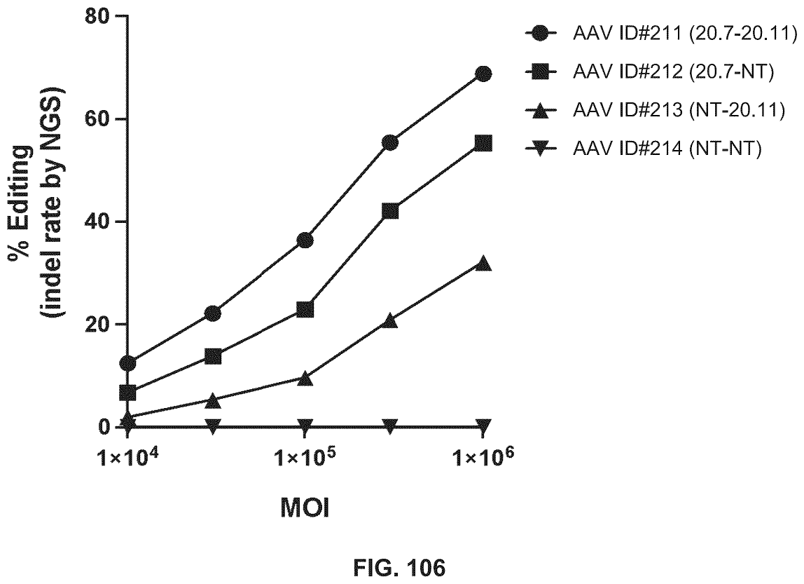

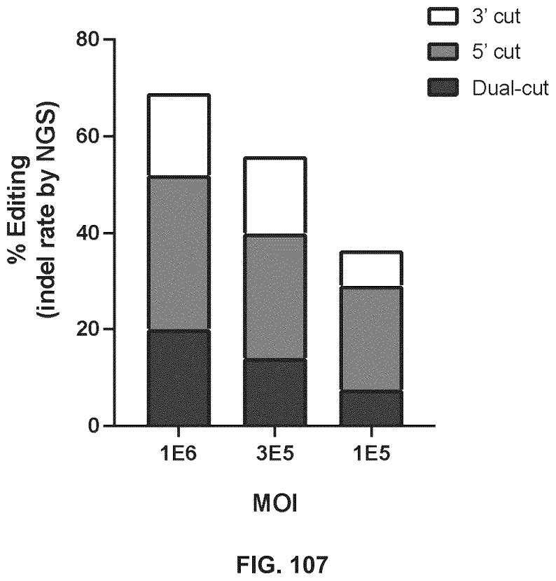

AAV constructs (Construct ID 211-214) assessed in FIGS. 106-107 were generated using methods described in Example 1. Sequences for these AAV plasmids are listed in Table 21.

TABLE 21
Sequences of AAV constructs with dual-guides targeting either side of the CTG
repeat in DMPK 3′ UTR. NT = non-targeting guide*

| Construct | | | SEQ ID |
|---|---|---|---|
| ID | Component Name | DNA Sequence | NO |
| 211 through | 5′ ITR | CCTGCAGGCAGCTGCGCGCTCGCTCGCTCACTGAGGCC | 423 |
| 214 | | GCCCGGGCGTCGGGCGACCTTTGGTCGCCCGGCCTCAG | |
| | | TGAGCGAGCGAGCGCGCAGAGAGGGAGTGGCCAACTCC | |
| | | ATCACTAGGGGTTCCT | |
| | buffer sequence | GCGGCCTCTAGACTCGAGGCGTT | 788 |
| | CMV enhancer + | GACATTGATTATTGACTAGTTATTAATAGTAATCAATT | |
| | promoter | ACGGGGTCATTAGITCATAGCCCATATATGGAGTTCCG | |
| | | CGTTACATAACTTACGGTAAATGGCCCGCCTGGCTGAC | |
| | | CGCCCAACGACCCCCGCCCATTGACGTCAATAATGACG | |
| | | TATGTTCCCATAGTAACGCCAATAGGGACTTTCCATTG | |
| | | ACGTCAATGGGTGGAGTATTTACGGTAAACTGCCCACT | |
| | | TGGCAGTACATCAAGTGTATCATATGCCAAGTACGCCC | |
| | | CCTATTGACGTCAATGACGGTAAATGGCCCGCCTGGCA | |
| | | TTATGCCCAGTACATGACCTTATGGGACTTTCCTACTT | |
| | | GGCAGTACATCTACGTATTAGTCATCGCTATTACCATG | |
| | | GTGATGCGGTTTTGGCAGTACATCAATGGGCGTGGATA | |
| | | GCGGTTTGACTCACGGGGATTTCCAAGTCTCCACCCCA | |
| | | TTGACGTCAATGGGAGTTTGTTTTGGCACCAAAATCAA | |
| | | CGGGACTTTCCAAAATGTCGTAACAACTCCGCCCCATT | |
| | | GACGCAAATGGGCGGTAGGCGTGTACGGTGGGAGGTCT | |
| | | ATATAAGCAGAGCT | |
| | buffer sequence | CTCTGGCTAACTACCGGT | 789 |
| | Kozak | GCCACC | NA |
| | start codon | ATGGCC | NA |
| | SV40 NLS | CCAAAGAAGAAGCGGAAGGTC | 790 |
| | linker | TCTAGA | NA |
| | CasX 491 | CAAGAGATCAAGAGAATCAACAAGATCAGAAGGAGACT | 791 |
| | | GGTCAAGGACAGCAACACAAAGAAGGCCGGCAAGACAG | |
| | | GCCCCATGAAAACCCTGCTCGTCAGAGTGATGACCCCT | |
| | | GACCTGAGAGAGCGGCTGGAAAACCTGAGAAAGAAGCC | |
| | | CGAGAACATCCCTCAGCCTATCAGCAACACCAGCAGGG | |
| | | CCAACCTGAACAAGCTGCTGACCGACTACACCGAGATG | |
| | | AAGAAAGCCATCCTGCACGTGTACTGGGAAGAGTTCCA | |
| | | GAAAGACCCCGTGGGCCTGATGAGCAGAGTTGCTCAGC | |
| | | CTGCCAGCAAGAAGATCGACCAGAACAAGCTGAAGCCC | |
| | | GAGATGGACGAGAAGGGCAATCTGACCACAGCCGGCTT | |
| | | TGCCTGCTCTCAGTGTGGCCAGCCTCTGTTCGTGTACA | |
| | | AGCTGGAACAGGTGTCCGAGAAAGGCAAGGCCTACACC | |
| | | AACTACTTCGGCAGATGTAACGTGGCCGAGCACGAGAA | |
| | | GCTGATTCTGCTGGCCCAGCTGAAACCTGAGAAGGACT | |
| | | CTGATGAGGCCGTGACCTACAGCCTGGGCAAGTTTGGA | |
| | | CAGAGAGCCCTGGACTTCTACAGCATCCACGTGACCAA | |
| | | AGAAAGCACACACCCCGTGAAGCCCCTGGCTCAGATCG | |
| | | CCGGCAATAGATACGCCTCTGGACCTGTGGGCAAAGCC | |
| | | CTGTCCGATGCCTGCATGGGAACAATCGCCAGCTTCCT | |
| | | GAGCAAGTACCAGGACATCATCATCGAGCACCAGAAGG | |
| | | TGGTCAAGGGCAACCAGAAGAGACTGGAAAGCCTGAGG | |
| | | GAGCTGGCCGGCAAAGAGAACCTGGAATACCCCAGCGT | |
| | | GACCCTGCCTCCTCAGCCTCACACAAAAGAAGGCGTGG | |
| | | ACGCCTACAACGAAGTGATCGCCAGAGTGAGAATGTGG | |
| | | GTCAACCTGAACCTGTGGCAGAAGCTGAAACTGTCCAG | |
| | | GGACGACGCCAAGCCTCTGCTGAGACTGAAGGGCTTCC | |
| | | CTAGCTTCCCTCTGGTGGAAAGACAGGCCAATGAAGTG | |
| | | GATTGGTGGGACATGGTCTGCAACGTGAAGAAGCTGAT | |
| | | CAACGAGAAGAAAGAGGATGGCAAGGTTTTCTGGCAGA | |
| | | ACCTGGCCGGCTACAAGAGACAAGAAGCCCTGAGGCCT | |
| | | TACCTGAGCAGCGAAGAGGACCGGAAGAAGGGCAAGAA | |
| | | GTTCGCCAGATACCAGCTGGGCGACCTGCTGCTGCACC | |
| | | TGGAAAAGAAGCACGGCGAGGACTGGGGCAAAGTGTAC | |
| | | GATGAGGCCTGGGAGAGAATCGACAAGAAGGTGGAAGG | |
| | | CCTGAGCAAGCACATTAAGCTGGAAGAGGAAAGAAGGA | |
| | | GCGAGGACGCCCAATCTAAAGCCGCTCTGACCGATTGG | |
| | | CTGAGAGCCAAGGCCAGCTTTGTGATCGAGGGCCTGAA | |
| | | AGAGGCCGACAAGGACGAGTTCTGCAGATGCGAGCTGA | |
| | | AGCTGCAGAAGTGGTACGGCGATCTGAGAGGCAAGCCC | |
| | | TTCGCCATTGAGGCCGAGAACAGCATCCTGGACATCAG | |
| | | CGGCTTCAGCAAGCAGTACAACTGCGCCTTCATTTGGC | |
| | | AGAAAGACGGCGTCAAGAAACTGAACCTGTACCTGATC | |
| | | ATCAATTACTTCAAAGGCGGCAAGCTGCGGTTCAAGAA | |
| | | GATCAAACCCGAGGCCTTCGAGGCTAACAGATTCTACA | |
| | | CCGTGATCAACAAAAAGTCCGGCGAGATCGTGCCCATG | |
| | | GAAGTGAACTTCAACTTCGACGACCCCAACCTGATTAT | |
| | | CCTGCCTCTGGCCTTCGGCAAGAGACAGGGCAGAGAGT | |
| | | TCATCTGGAACGATCTGCTGAGCCTGGAAACCGGCTCT | |
| | | CTGAAGCTGGCCAATGGCAGAGTGATCGAGAAAACCCT | |
| | | GTACAACAGGAGAACCAGACAGGACGAGCCTGCTCTGT | |
| | | TTGTGGCCCTGACCTTCGAGAGAAGAGAGGTGCTGGAC | |
| | | AGCAGCAACATCAAGCCCATGAACCTGATCGGCGTGGA | |
| | | CCGGGGCGAGAATATCCCTGCTGTGATCGCCCTGACAG | |
| | | ACCCTGAAGGATGCCCACTGAGCAGATTCAAGGACTCC | |
| | | CTGGGCAACCCTACACACATCCTGAGAATCGGCGAGAG | |
| | | CTACAAAGAGAAGCAGAGGACAATCCAGGCCAAGAAAG | |
| | | AGGTGGAACAGCGCAGAGCCGGCGGATACTCTAGGAAG | |
| | | TACGCCAGCAAGGCCAAGAATCTGGCCGACGACATGGT | |
| | | CCGAAACACCGCCAGAGATCTGCTGTACTACGCCGTGA | |
| | | CACAGGACGCCATGCTGATCTTCGAGAATCTGAGCAGA | |
| | | GGCTTCGGCCGGCAGGGCAAGAGAACCTTTATGGCCGA | |
| | | GAGGCAGTACACCAGAATGGAAGATTGGCTCACAGCTA | |
| | | AACTGGCCTACGAGGGACTGAGCAAGACCTACCTGTCC | |
| | | AAAACACTGGCCCAGTATACCTCCAAGACCTGCAGCAA | |
| | | TTGCGGCTTCACCATCACCAGCGCCGACTACGACAGAG | |
| | | TGCTGGAAAAGCTCAAGAAAACCGCCACCGGCTGGATG | |
| | | ACCACCATCAACGGCAAAGAGCTGAAGGTTGAGGGCCA | |
| | | GATCACCTACTACAACAGGTACAAGAGGCAGAACGTCG | |
| | | TGAAGGATCTGAGCGTGGAACTGGACAGACTGAGCGAA | |
| | | GAGAGCGTGAACAACGACATCAGCAGCTGGACAAAGGG | |
| | | CAGATCAGGCGAGGCTCTGAGCCTGCTGAAGAAGAGGT | |
| | | TTAGCCACAGACCTGTGCAAGAGAAGTTCGTGTGCCTG | |
| | | AACTGCGGCTTCGAGACACACGCCGATGAACAGGCTGC | |
| | | CCTGAACATTGCCAGAAGCTGGCTGTTCCTGAGAAGCC | |
| | | AAGAGTACAAGAAGTACCAGACCAACAAGACCACCGGC | |
| | | AACACCGACAAGAGGGCCTTTGTGGAAACCTGGCAGAG | |
| | | CTTCTACAGAAAAAAGCTGAAAGAAGTCTGGAAGCCCG | |
| | linker | GGATCC | NA |
| | SV40 NLS | CCAAAAAAGAAGAGAAAGGTA | 792 |
| | HA tag | TACCCATATGATGTCCCTGACTACGCT | 793 |
| | linker + stop | GGATCCTAA | NA |
| | codon | | |
| | buffer sequence | GAATTCCTAGAGCTCGCTGATCAGCCTCGA | 794 |
| | bGH poly(A) | CTGTGCCTTCTAGTTGCCAGCCATCTGTTGTTTGCCCC | 514 |
| | signal | TCCCCCGTGCCTTCCTTGACCCTGGAAGGTGCCACTCC | |
| | | CACTGTCCTTTCCTAATAAAATGAGGAAATTGCATCGC | |
| | | ATTGTCTGAGTAGGTGTCATTCTATTCTGGGGGGTGGG | |
| | | GTGGGGCAGGACAGCAAGGGGGAGGATTGGGAAGAGAA | |
| | | TAGCAGGCATGCTGGGGA | |
| | buffer sequence | GGTACCGT | NA |
| | U6 promoter | GAGGGCCTATTTCCCATGATTCCTTCATATTTGCATAT | 661 |
| | | ACGATACAAGGCTGTTAGAGAGATAATTGGAATTAATT | |
| | | TGACTGTAAACACAAAGATATTAGTACAAAATACGTGA | |
| | | CGTAGAAAGTAATAATTTCTTGGGTAGTTTGCAGTTTT | |
| | | AAAATTATGTTTTAAAATGGACTATCATATGCTTACCG | |
| | | TAACTTGAAAGTATTTCGATTTCTTGGCTTTATATATC | |
| | | TTGTGGAAAGGAC | |
| | buffer sequence | GAAACACC | NA |
| | Scaffold 174 | ACTGGCGCTTTTATCTGATTACTTTGAGAGCCATCACC | 691 |
| | | AGCGACTATGTCGTAGTGGGTAAAGCTCCCTCTTCGGA | |
| | | GGGAGCATCAAAG | |
| | Spacer 1 | See specific dual guide combos below | — |
| | (5′ CTG) | | |
| | buffer sequence | TTTTTTTTGGCTAGC | 4004 |
| | U6 promoter | GAGGGCCTATTTCCCATGATTCCTTCATATTTGCATAT | 661 |
| | | ACGATACAAGGCTGTTAGAGAGATAATTGGAATTAATT | |
| | | TGACTGTAAACACAAAGATATTAGTACAAAATACGTGA | |
| | | CGTAGAAAGTAATAATTTCTTGGGTAGTTTGCAGTTTT | |
| | | AAAATTATGTTTTAAAATGGACTATCATATGCTTACCG | |
| | | TAACTTGAAAGTATTTCGATTTCTTGGCTTTATATATC | |
| | | TTGTGGAAAGGAC | |
| | buffer sequence | GAAACACC | NA |
| | Scaffold 174 | ACTGGCGCTTTTATCTGATTACTTTGAGAGCCATCACC | 691 |
| | | AGCGACTATGTCGTAGTGGGTAAAGCTCCCTCTTCGGA | |
| | | GGGAGCATCAAAG | |
| | Spacer 2 | See specific dual guide combos below | — |
| | (3′ CTG) | | |
| | buffer sequence | TTTTTTTTGGCGGCCGC | 796 |
| | 3′ ITR | AGGAACCCCTAGTGATGGAGTTGGCCACTCCCTCTCTG | 424 |
| | | CGCGCTCGCTCGCTCACTGAGGCCGGGCGACCAAAGGT | |
| | | CGCCCGACGCCCGGGCTTTGCCCGGGCGGCCTCAGTGA | |
| | | GCGAGCGAGCGCGCAGCTGCCTGCAGG | |
| 211 | Spacer 1 (20.7) | CGGCTACAAGGACCCTTCGA | 4005 |
| | Spacer 2 (20.11) | CAGGCCTGCAGTTTGCCCAT | 4006 |
| 212 | Spacer 1 (20.7) | CGGCTACAAGGACCCTTCGA | 4005 |
| | Spacer 2 (NT) | GGGTCTTCGAGAAGACCC | 537 |
| 213 | Spacer 1 (NT) | GGGTCTTCGAGAAGACCC | 537 |
| | Spacer 2 (20.11) | CAGGCCTGCAGTTTGCCCAT | 4006 |
| 214 | Spacer 1 (NT) | GGGTCTTCGAGAAGACCC | 537 |
| | Spacer 2 (NT) | GGGTCTTCGAGAAGACCC | 537 |

*Components are listed in a 5′ to 3′ order within the constructs

Production of AAV vectors from AAV constructs 211-214 and subsequent titering were performed as described in Example 1.

### AAV Transduction of HEK293T Cells:

˜10,000 HEK293T cells per well were seeded in 96-well plates. 24 hours later, seeded cells were treated with AAVs encoding CasX variant 491 with the dual-guide system (i.e., scaffold 174 with spacers 20.7-20.11, 20.7-NT, NT-20.11, or NT-NT; refer to Table 21 for sequences). Viral infection conditions were performed in triplicate, with normalized number of viral genomes (vg) among experimental vectors, in a series of three-fold dilution of multiplicity of infection (MOI) ranging from ˜1E6 to 1E4 vg/cell. Five days post-transduction, AAV-treated HEK293T cells were harvested for gDNA extraction for editing analysis at the DMPK locus by NGS. Briefly, amplicons were amplified from 200 ng of extracted gDNA with a set of primers targeting the CTG repeat region in the DMPK 3′ UTR and processed as described in Example 23.

### Results:

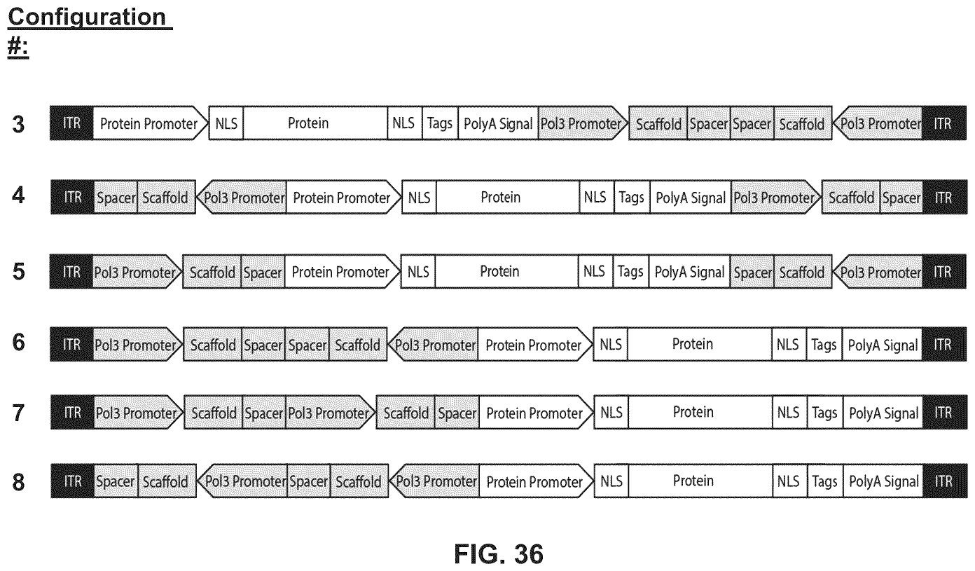

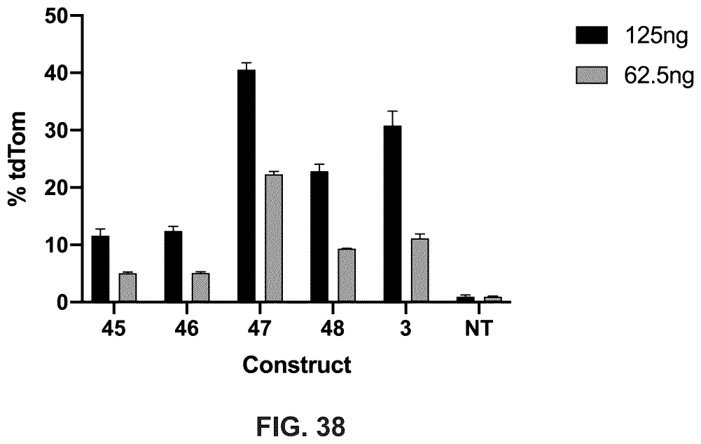

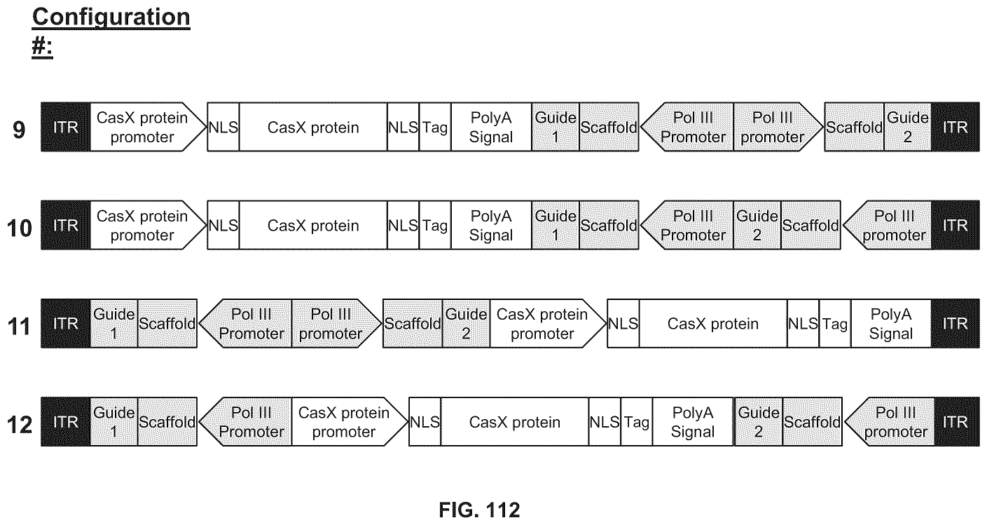

FIG. 35 is a schematic of two AAV construct configurations (architecture 1 and architecture 2). FIG. 36 and FIG. 112 show additional AAV construct configurations. FIG. 37 depicts the specific dual-spacer combinations. The results of the editing assay portrayed in FIG. 38 demonstrate that the constructs delivered as AAV transgene plasmids to mNPCs in architecture 2 edit with enhanced potency. The results from the assay assessing the different combinations of targeting and non-targeting spacers demonstrate that each individual gRNA was active, although, architectures with one targeting spacer and one non-targeting spacer (constructs AAV45 and AAV46) yielded approximately 18% lower editing levels. Certain combinations of targeting spacers yielded increased efficacy. While use of the dual-spacer combination 12.7-12.2 (construct AAV48) resulted in editing with significant potency, use of two sets of 12.7 spacers (construct AAV47) resulted in editing with 10% greater potency than that seen with the single gRNA architecture of construct AAV3 (FIG. 38).

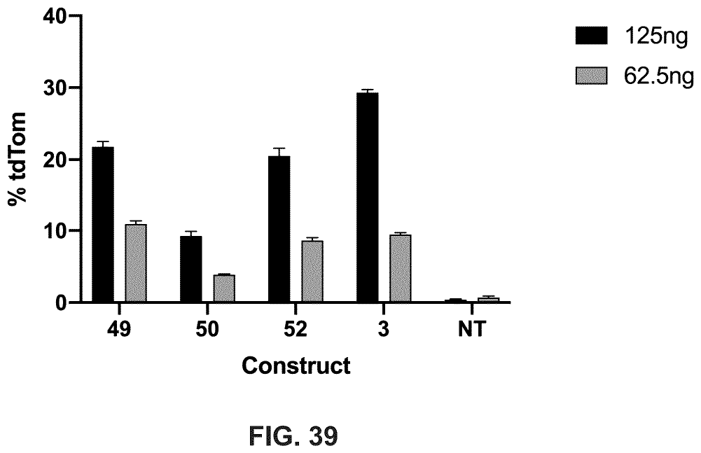

The bar plot in FIG. 39 shows the results that use of AAV constructs 49, 50, and 52, which had the arrangements where two gRNA transcriptional units were placed on either side of the CasX gene, were also able to edit the target nucleic acid when delivered to mNPCs.

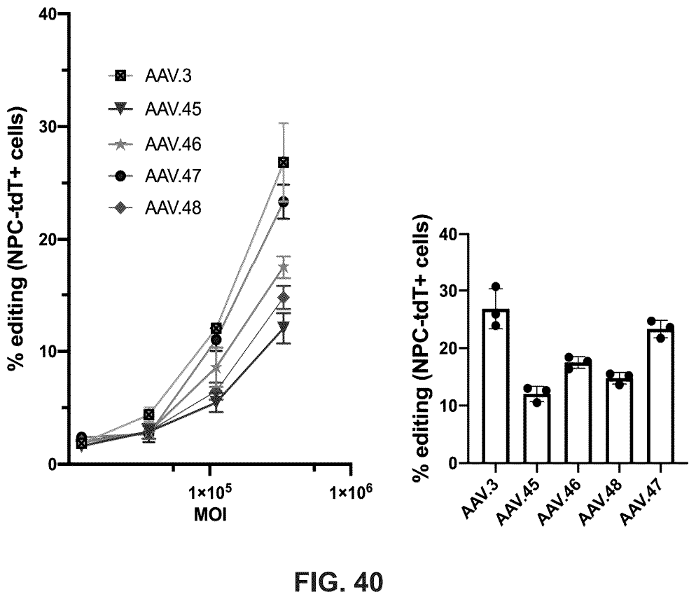

The plots in FIG. 40 show the results that use of AAV constructs 3, 45, 46, 47, and 48, delivered as AAVs, were able to edit the target stop cassette in mNPCs. Each vector displayed dose-dependent editing at the target locus (FIG. 40, left panel). At an MOI of 3e5, AAV.47 had <5% less potency than the original orientation vector AAV.3 (FIG. 40, right panel).

Experiments were also performed to demonstrate the use of CasX and a dual-guide system in targeting and excising the CTG repeat in the 3′UTR region of the human DMPK gene. The significance of evaluating the ability to target this repeat is that the neuromuscular disease myotonic dystrophy type 1 (DM1) is caused by the abnormal CTG repeat expansion in the 3′ noncoding region of the human DMPK gene. Here, HEK293T cells were transduced with dual-guide AAVs harboring either two DMPK-targeting spacers (20.7 and 20.11), the combination of one DMPK-targeting spacer and one non-targeting (NT) spacer (20.7 and NT or NT and 20.11), or two non-targeting spacers (NT-NT) at various MOIs. The results shown in FIG. 106 demonstrate on-target editing at either side or both sides flanking the CTG repeat expansion in transduced HEK293T cells occurred in a dose-dependent manner. The highest level of indel rate was attained with the dual-guide AAV (spacers 20.7 and 20.11), reaching ˜70% editing efficiency at the highest MOI of 1E6 vg/cell. In addition, infecting cells with AAVs expressing the combination of one DMPK-targeting spacer and one NT spacer (20.7 and NT or NT and 20.11) revealed that a higher editing efficiency was achieved on the 5′ region (by spacer 20.7 and NT) of the CTG repeat in comparison to editing on the 3′ region (by spacer NT and 20.11) (FIG. 106). FIG. 107 illustrates the quantification of percent editing of indel rate detected by NGS for the various types of editing (i.e., editing at 5′ or 3′ of CTG repeat, or dual-editing resulting in dropout of CTG repeat) induced by the AAVs harboring two DMPK-targeting spacers (20.7-20.11). Double-cut editing resulting in CTG repeat excision occurred in a dose-dependent manner, with 21% excision rate achieved at the highest MOI of 1E6 (FIG. 107). High levels of editing were similarly observed at the individual 5′ or 3′ region of the CTG repeat, with a majority of indel events occurring in the 5′ region.

Altogether, these experiments demonstrate the feasibility of using dual gRNAs in combination with the full CasX protein sequence in a single AAV, which would not be achievable with the use of larger CRISPR proteins, such as Cas9, due to the packaging constraints of the AAV capsid. The experiments also show that dual guide RNAs in an all-in-one vector construct were able to retain the ability to edit the target nucleic acid. Furthermore, the results demonstrate the ability to package and deliver CasX with the dual-guide system from an all-in-one single AAV vector in vitro, which resulted in efficient editing and excision of the target genomic region. In addition to using a dual-guide system to excise a target genomic region, combining two gRNA transcriptional units could also provide the ability to 1) increase gRNA expression and thus CasX-mediated editing or 2) target two distinct genes that might have cooperative therapeutic effects. The effects of varying the orientation and position of gRNA promoters are further investigated in Example 37.

### Example 10: Nuclear Localization Sequence (NLS) Selection Enhances Small CRISPR Protein Potency

This experiment shows that alteration of the nuclear localization sequence (NLS) utilized in AAV constructs comprising CasX and gRNA can modulate editing.

### Materials and Methods

AAV vectors were cloned and produced according to standard methods, which are described in Example 1. The amino acid sequences of the encoded NLS are presented in Table 22 and Table 23.

Methods for production of AAV vectors and nucleofection were conducted as described in Example 1. The sequences of the additional components of AAV constructs, with the exception of sequences encoding the CasX (Table 63) and the one or more gRNA (Table 26), are listed in Table 64.

TABLE 22
N-terminal NLS sequences

| | | SEQ ID |
|---|---|---|
| NLS Amino Acid Sequence* | NLS ID | NO |
| PKKKRKVSR | 1 | 538 |
| PKKKRKVGGSPKKKRKVGGSPKKKRKVGGSPKKKRKVSR | 2 | 539 |
| PKKKRKVGGSPKKKRKVGGSPKKKRKVGGSPKKKRKVGGSPKKKRKVGGSPKKKRKVSR | 3 | 540 |
| PAAKRVKLDSR | 4 | 541 |
| PAAKRVKLDGGSPAAKRVKLDSR | 5 | 542 |
| PAAKRVKLDGGSPAAKRVKLDGGSPAAKRVKLDGGSPAAKRVKLDSR | 6 | 543 |
| PAAKRVKLDGGSPAAKRVKLDGGSPAAKRVKLDGGSPAAKRVKLDGGSPAAKRVKLDGGSPA | 7 | 544 |
| AKRVKLDSR | | |
| KRPAATKKAGQAKKKKSR | 8 | 545 |
| KRPAATKKAGQAKKKKGGSKRPAATKKAGOAKKKKSR | 9 | 546 |
| PAAKRVKLDGGSPKKKRKVSR | 10 | 547 |
| PAAKKKKLDGGSPKKKRKVSR | 11 | 548 |
| PAAKKKKLDSR | 12 | 549 |
| PAAKKKKLDGGSPAAKKKKLDGGSPAAKKKKLDSR | 13 | 550 |
| PAAKKKKLDGGSPAAKKKKLDGGSPAAKKKKLDGGSPAAKKKKLDSR | 14 | 551 |
| PAKRARRGYKCSR | 15 | 552 |
| PAKRARRGYKCGSPAKRARRGYKCSR | 16 | 553 |
| PRRKREESR | 17 | 554 |
| PYRGRKESR | 18 | 555 |
| PLRKRPRRSR | 19 | 556 |
| PLRKRPRRGSPLRKRPRRSR | 20 | 557 |
| PAAKRVKLDGGKRTADGSEFESPKKKRKVGGS | 21 | 558 |
| PAAKRVKLDGGKRTADGSEFESPKKKRKVPPPPG | 22 | 559 |
| PAAKRVKLDGGKRTADGSEFESPKKKRKVGIHGVPAAPG | 23 | 560 |
| PAAKRVKLDGGKRTADGSEFESPKKKRKVGGGSGGGSPG | 24 | 561 |
| PAAKRVKLDGGKRTADGSEFESPKKKRKVPGGGSGGGSPG | 25 | 562 |
| PAAKRVKLDGGKRTADGSEFESPKKKRKVAEAAAKEAAAKEAAAKAPG | 26 | 563 |
| PAAKRVKLDGGKRTADGSEFESPKKKRKVPG | 27 | 564 |
| PAAKRVKLDGGSPKKKRKVGGS | 28 | 565 |
| PAAKRVKLDPPPPKKKRKVPG | 29 | 566 |
| PAAKRVKLDPG | 30 | 567 |
| PAAKRVKLDGGGSGGGSGGGS | 31 | 568 |
| PAAKRVKLDPPP | 32 | 569 |
| PAAKRVKLDGGGSGGGSGGGSPPP | 33 | 570 |
| PKKKRKVPPP | 34 | 571 |
| PKKKRKVGGS | 35 | 572 |
| MAPKKKRKVSR | 36 | 771 |
| MAPKKKRKVGGSPKKKRKVGGSPKKKRKVGGSPKKKRKVSR | 37 | 772 |

*Sequences in bold are NLS, while unbolded sequences are linkers.

TABLE 23
C-terminal NLS sequences

| | | SEQ ID |
|---|---|---|
| NLS Amino Acid Sequence* | NLS ID | NO |
| GSPKKKRKV | 1 | 573 |
| GSPKKKRKVGGSPKKKRKVGGSPKKKRKVGGSPKKKRKV | 2 | 574 |
| GSPKKKRKVGGSPKKKRKVGGSPKKKRKVGGSPKKKRKVGGSPKKKRKVGGSPKKKRKV | 3 | 575 |
| GSPAAKRVKLD | 4 | 576 |
| GSPAAKRVKLDGGSPAAKRVKLD | 5 | 577 |
| GSPAAKRVKLDGGSPAAKRVKLDGGSPAAKRVKLDGGSPAAKRVKLD | 6 | 578 |
| GSPAAKRVKLDGGSPAAKRVKLDGGSPAAKRVKLDGGSPAAKRVKLDGGSPAAKRVKLDGGS | 7 | 579 |
| PAAKRVKLD | | |
| GSKRPAATKKAGQAKKKK | 8 | 580 |
| KRPAATKKAGQAKKKKGGSKRPAATKKAGQAKKKK | 9 | 581 |
| GSPAAKRVKLGGSPAAKRVKLGGSPKKKRKVGGSPKKKRKV | 10 | 582 |
| GSKLGPRKATGRWGS | 11 | 583 |
| GSKRKGSPERGERKRHWGS | 12 | 584 |
| GSPKKKRKVGSGSKRPAATKKAGQAKKKKLE | 13 | 585 |
| GPKRTADSQHSTPPKTKRKVEFEPKKKRKV | 14 | 586 |
| GGGSGGGSKRTADSQHSTPPKTKRKVEFEPKKKRKV | 15 | 587 |
| AEAAAKEAAAKEAAAKAKRTADSQHSTPPKTKRKVEFEPKKKRKV | 16 | 588 |
| GPPKKKRKVGGSKRTADSQHSTPPKTKRKVEFEPKKKRKV | 17 | 589 |
| GPAEAAAKEAAAKEAAAKAPAAKRVKLD | 18 | 590 |
| GPGGGSGGGSGGGSPAAKRVKLD | 19 | 591 |
| GPPKKKRKVPPPPAAKRVKLD | 20 | 592 |
| GPPAAKRVKLD | 21 | 593 |
| GSPKKKRKV | 22 | 594 |
| GSPAAKRVKLD | 23 | 595 |
| VGSKRPAATKKAGQAKKKK | 24 | 596 |
| TGGGPGGGAAAGSGSPKKKRKVGSGSKRPAATKKAGOAKKKKLE | 25 | 597 |
| GGGPGGGAAAGSGSPKKKRKVGSGSKRPAATKKAGQAKKKKLE | 26 | 844 |
| TGGGPGGGAAAGSGSPKKKRKVGSGS | 27 | 599 |
| PPPPKKKRKVPPP | 28 | 600 |
| GGSPKKKRKVPPP | 29 | 601 |
| PPPPKKKRKV | 30 | 602 |
| GGSPKKKRKV | 31 | 603 |
| GGSPKKKRKVGGSGGSGGS | 32 | 604 |
| GGSPKKKRKVGGSPKKKRKV | 33 | 605 |
| GGSGGSGGSPKKKRKVGGSPKKKRKV | 34 | 606 |
| VGGGSGGGSGGGSPAAKRVKLD | 35 | 607 |
| VPPPPAAKRVKLD | 36 | 608 |
| VPPPGGGSGGGSGGGSPAAKRVKLD | 37 | 609 |
| VGGGSGGGSGGGSPAAKRVKLD | 38 | 610 |
| PPPPAAKRVKLD | 39 | 845 |
| PPPGGGSGGGSGGGSPAAKRVKLD | 40 | 846 |
| VGSPAAKRVKLD | 41 | 613 |

*Sequences in bold are NLS, while un-bolded sequences are linkers.

AAV transduction and editing level assessment in mNPTC-tdT cells by FACS were conducted as described in Example 1.

### Results:

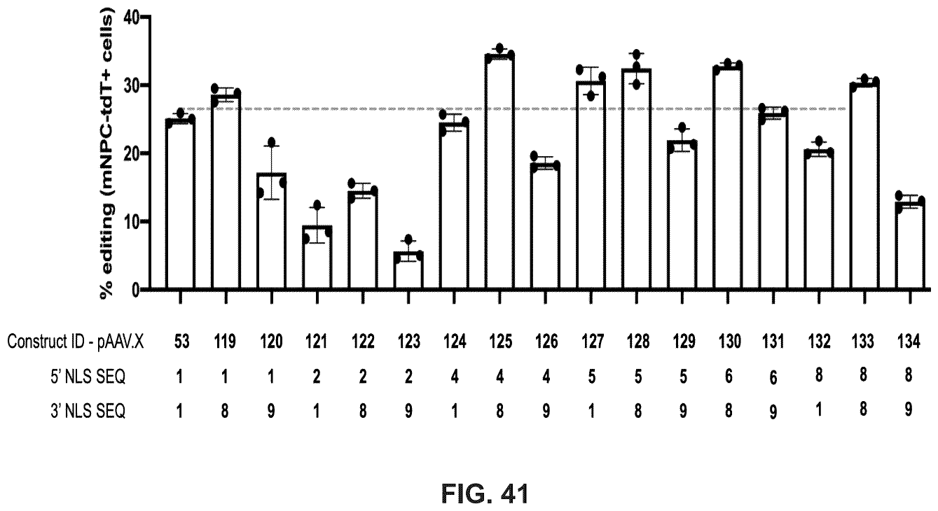

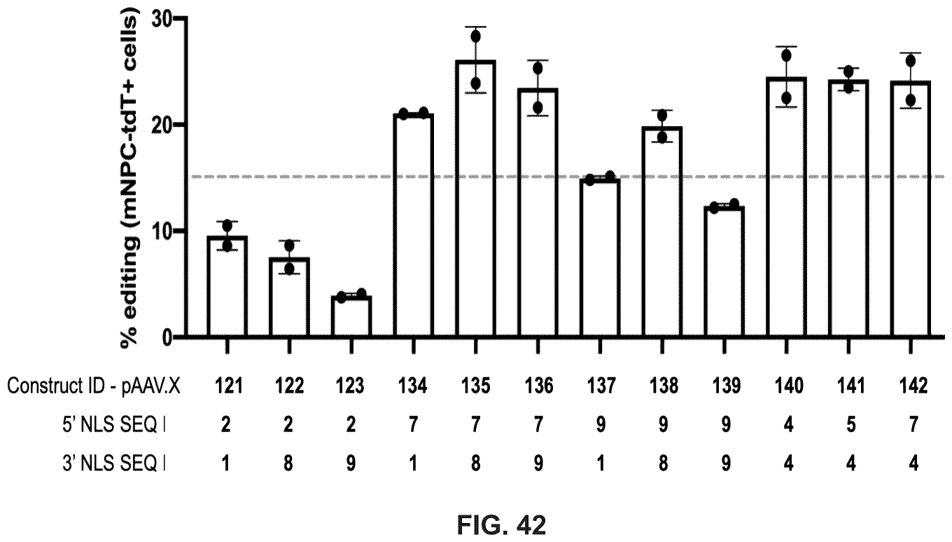

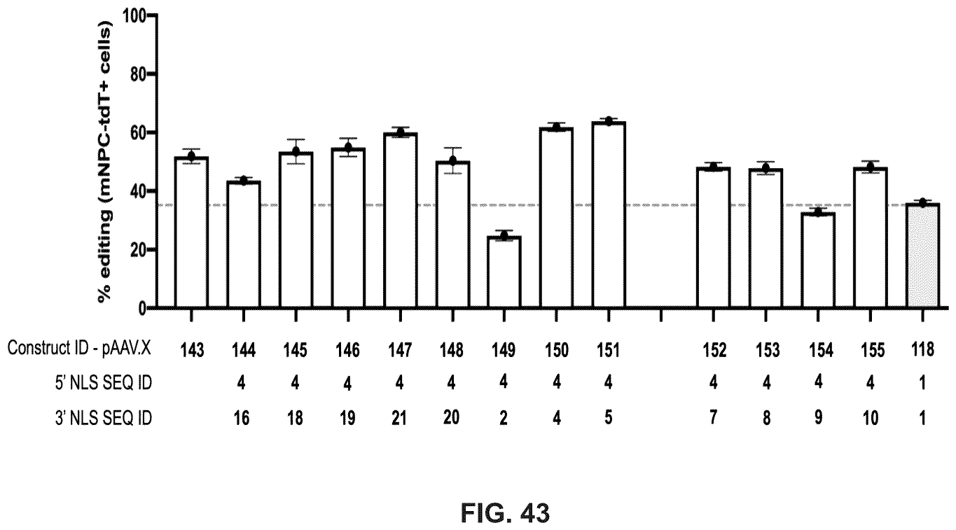

Initial plasmid nucleofection revealed that a number of NLS permutations displayed improved editing when compared to control (1×SV40 NLS on both the N- and C-termini). In particular, N-terminal variants containing Cmyc or Nucleoplasmin NLSs significantly outperformed SV40 NLS combinations (FIG. 41, NLS numbers in FIGS. 41-42 refer to the NLS ID numbers in Table 22 and Table 23). This trend in N-terminal NLS variation was replicated in AAV transduction, where Cmyc and Nucleoplasmin NLS variants again outperformed SV40 NLS variants (FIG. 42). Finally, variations holding the Cmyc constant (FIG. 43) were tested, and the results demonstrate that the constructs with the highest level of editing contained a Cmyc NLS on both the N- and C-terminals.

The data show that selecting the amino acid sequence of the NLS can enhance editing outcomes in the AAV setting. Specifically, N-terminal Cmyc-containing NLS variants showed a clear improvement compared to N-terminal SV40 NLS variants. In addition, C-terminal Cmyc and Nuc variants improve editing over SV40 NLS variants. Repetitions of the SV40 NLS seem to be deleterious for editing efficiency on both the N- and C-terminals.

### Example 11: Introns in the 5′ UTR can Enhance Small CRISPR Protein Expression

This experiment demonstrates that transcriptional levels mediated by AAV vectors delivering small CRISPR proteins (such as CasX) can be enhanced by inclusion of different regulatory elements such as intronic sequences taken from viral, mouse, or human genomes that do not fit in AAV vectors expressing large transgene (e.g., spCas9) plasmids.

### Methods:

AAV cloning and production are as described in Example 1. 5′ sequences used to generate the AAV cis plasmid contain protein promoters including UbC, JeT, CMV, CAG, CBH, hSyn, or another Pol II promoter, intronic region, and N-terminal NLS, while 3′ sequences contain C-terminal NLS, poly A signal, RNA promoter and guide RNA containing spacer 12.7. Non-limiting examples of intron sequences to be incorporated into the constructs are listed in Table 24.

Enhancement in editing by the inclusion of intron 36 (transgene plasmid 59) is tested against transgene plasmid 58, which was the baseline construct not containing the intron. The rest of the introns in Table 24 have been derived from viral, mouse, and human origin.

TABLE 24
Intron sequences for incorporation into base construct AAV58

| Intron | Size (bp) | SEQ ID NO |
|---|---|---|
| 1 | 54 | 614 |
| 2 | 67 | 615 |
| 3 | 62 | 616 |
| 4 | 49 | 617 |
| 5 | 59 | 618 |
| 6 | 67 | 619 |
| 7 | 66 | 620 |
| 8 | 86 | 621 |
| 9 | 67 | 622 |
| 10 | 70 | 623 |
| 11 | 476 | 624 |
| 12 | 69 | 625 |
| 13 | 69 | 626 |
| 14 | 70 | 627 |
| 15 | 70 | 628 |
| 16 | 108 | 629 |
| 17 | 69 | 630 |
| 18 | 206 | 631 |
| 19 | 70 | 632 |
| 20 | 68 | 633 |
| 21 | 299 | 634 |
| 22 | 226 | 635 |
| 23 | 71 | 636 |
| 24 | 69 | 637 |
| 25 | 87 | 638 |
| 26 | 84 | 639 |
| 27 | 82 | 640 |
| 28 | 66 | 641 |
| 29 | 65 | 642 |
| 30 | 66 | 643 |
| 31 | 106 | 644 |
| 32 | 69 | 645 |
| 33 | 68 | 646 |
| 34 | 68 | 647 |
| 35 | 97 | 648 |
| 36 | 140 | 649 |
| 37 | 133 | 650 |
| 38 | 190 | 651 |
| 39 | 271 | 652 |
| 40 | 96 | 653 |
| 41 | 110 | 654 |
| 42 | 270 | 655 |
| 43 | 116 | 656 |
| 44 | 67 | 657 |
| 45 | 66 | 658 |

### Results:

The effects of introns on transgene expression are assessed by cloning 50 different introns into AAV-cis plasmid and then assaying for editing in the tdTomato assay used in the Examples supra.

When compared to the base construct without an intron, the addition of an intronic sequence increases the overall editing efficiency of AAV transgenes.

The results are expected to support that the addition of introns to siAAV-transgenes expressing CasX under the control of short but strong promoter sequences will enable increased CasX expression and on-target editing while reducing cargo size, further optimizing the AAV system.

### Example 12: Self-Targeting Alternative Linked Loci (STALL) Efficiency can be Regulated by Using Alternative PAMs in AAV Constructs

A self-inactivating AAV-CRISPR (siAAV-CRISPR) system was designed and evaluated for its ability to progressively decrease expression of the CRISPR nuclease after achieving the desirable editing outcome. The siAAV-CRISPR system included a self-targeting AAV-CRISPR that was designed such that it could be modulated by the incorporation of alternative protospacer adjacent motif (PAM) sequences adjacent to self-limiting segments incorporated into the construct, thereby mediating the levels of editing and self-inactivation kinetics. The use of weaker PAMs in the self-limiting segments of the vector delays cleavage of the self-limiting segments in comparison to the more active PAM utilized by the nuclease to edit the genomic target and, therefore, permits a longer duration of CasX expression prior to self-targeting and cleavage of the CasX, halting its ability to edit the spacer site on the genome. These experiments assessed differential activity constructs with the weaker PAMs and self-limiting segments. An overview of the AAV vector used in this design strategy for an siAAV-CRISPR system is shown in FIG. 44.

### Materials and Methods

### AAV Plasmid Cloning:

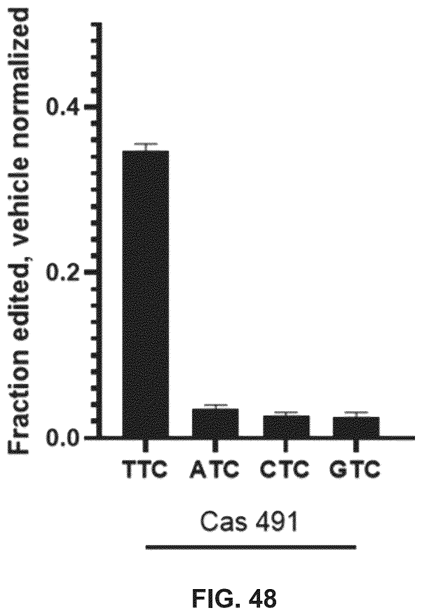

Each part in the AAV genome was separated by restriction enzyme sites to allow for modular cloning. A pAAV plasmid (construct 31) expressing CasX 491 or 676 under the control of a CMV promoter, with guide scaffold 174 and spacer 12.7 (CUGCAUUCUAGUUGUGGUUU, SEQ ID NO: 2860) under the control of the human U6 promoter was used to test the self-inactivation system modulated by various PAMs. Inserted in between the Pol II promoter and the sequence encoding the CasX protein was an identical copy of spacer 12.7, targeting the complementary strand orientation of the tdTomato locus with various appended PAM recognition motifs (TTC, construct 24; ATC, construct 25; CTC, construct 26; GTC, construct 27), as well as a scramble PAM that is not recognized by CasX (GGGG, construct 28). These various components were cloned into an AAV plasmid flanked by AAV2 ITRs following standard molecular cloning techniques. Cloned and sequence-validated constructs were maxi-prepped and subjected to quality assessment prior to AAV vector production using HEK293 Ts. The resulting constructs were used in experiments to generate the results shown in FIGS. 48, 49A-49B, and 50A-50B.

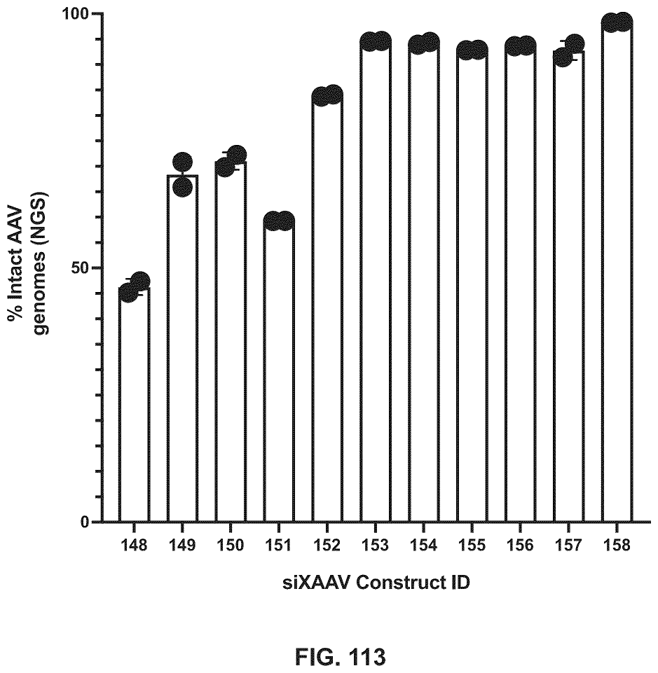

To obtain the results shown in FIG. 113, siAAV plasmids with construct IDs 148-158 were generated using similar methods as described above. Briefly, these siAAV plasmids expressed CasX 491 under the control of a U1A promoter, with guide scaffold 235 and a non-targeting spacer under the control of a human U6 promoter were generated to test the self-inactivation system modulated by the alternative PAMs. Inserted just upstream of the KOZAK sequence in between the Pol II promoter and the sequence encoding the CasX protein was an identical copy of a non-targeting spacer with various appended PAM recognition motifs (TTC, construct 148; ATC, construct 149; CTC, construct 150; GTC, construct 151; TTT construct 152; GTT construct 153; CTT construct 154; ATT construct 155; TGT construct 156; and CGC construct 157), as well as a control construct that did not contain a STALL site (construct 158). These various components were cloned into an AAV plasmid flanked by AAV2 ITRs following standard molecular cloning methods.

### AAV Vector Production was Performed as Described in Example 1.

To determine the viral genome (vg) titer, 1p L from crude lysate or column-purified virus was determined by qPCR using a set of primers and a probe specific for the CMV promoter or a 62 bp fragment located in the AAV2-ITR. Ten-fold serial dilutions of an AAV ITR plasmid was used as reference standards to calculate the titer (vg/mL) of viral samples.

Assessment of ssAAV Genome by NGS:

To assess the presence of indels in the packaged ssAAV genome, ssDNA was isolated from crude lysate or purified viruses by DNase I digest followed by Proteinase K incubation. 1-5 μL of ssDNA was used for amplification of the AAV transgene region flanking the self-inactivating off-target spacer. The amplified DNA was then bead-purified (Beckman Coulter, Agencourt Ampure XP) and re-amplified to incorporate the Illumina™ adapter sequence. Specifically, these primers contained an additional sequence at the 5′ end to introduce an Illumina™ adapter and a 16-nucleotide unique molecular identifier (UMI). Quality and quantification of the amplicon were assessed using a Fragment Analyzer DNA Analysis kit (Agilent, dsDNA 35-1500 bp). Amplicons were sequenced on an Illumina™ Miseq™ according to the manufacturer's instructions. Raw fastq files from sequencing were quality-controlled and processed using cutadapt v2.1 and flash2 v2.2.00, and CRISPResso2 v2.0.29. Each sequence was quantified for containing an insertion or deletion (indel) relative to the reference sequence, in a window around the 3′ end of the spacer (30 bp window centered at −3 bp from 3′ end of spacer). CasX activity was quantified as the total percent of reads that contain insertions, substitutions, and/or deletions anywhere within this window for each sample.

### PASS System to Assess CasX Variant Activity:

A multiplexed pooled approach was performed to assay protein variants using the PASS system. Briefly, a pooled HEK cell line was generated and termed PASS_V1.01. Each cell within the pool contained a genome-integrated single-guide RNA (sgRNA), paired with a specific target site. After transfection of protein-expression constructs, editing at a specific target by a specific spacer could be quantified by NGS. Each guide-target pair was designed to provide data related to activity, specificity, and targetability of the CasX-guide RNP complex. Fraction editing was normalized to a vehicle control.

### Results:

FIG. 48 shows the results of an editing assay assessing editing efficiency of CasX nuclease 491 in a custom HEK293 cell line, PASS_V1.01 assessing on-target editing sites at target sites consisting of the following PAM sequences: 48 TTC, 14 ATC, 22 CTC, and 11 GTC individual sites. Across 4 spacers, the TTC PAM spacers led to higher on-target editing (35% editing), followed by CTC and ATC PAM spacers (˜8% editing), followed by GTC PAM spacers, displaying the weakest editing (5%).

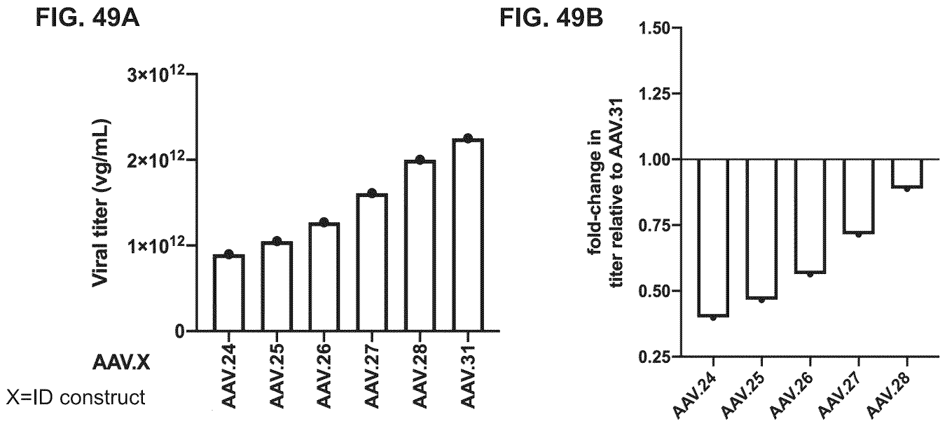

Differences in PAM-mediated inactivation were assessed in self-cleaving assays assessing AAV yield in production runs. Target spacer 12.7 was inserted in front of different PAMs (TTC, CTC, ATC, GTC, GGG) at the junction between the promoter and protein of pAAV.31 in constructs with a single guide targeting 12.7. The resulting siAAV vectors were produced and the titer of packaged viral genomes was quantified. The viral yield (vg/mL) correlated with the strength of the PAM used for the self-limiting segments utilized in the system (FIG. 49A). AAV.24 (TTC PAM) had the lowest viral yield after production, reflecting the higher rate of self-cleavage of the pAAV transgene during production and, therefore, reduction of encapsulation of the transgene into the AAV capsids compared to AAV control (AAV.31, FIG. 49B). The predicted “weakest” PAM, GTC (ID=27) led to a higher fraction of packaged siAAV, reflecting lower rate of self-cleavage during production.

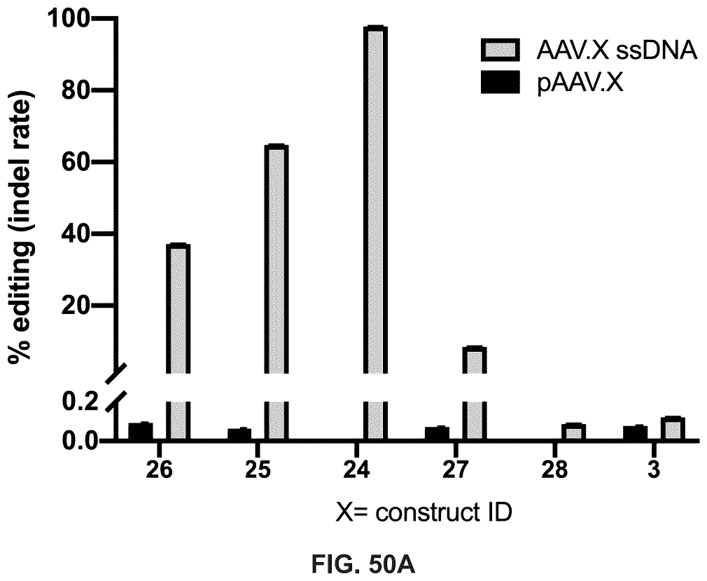

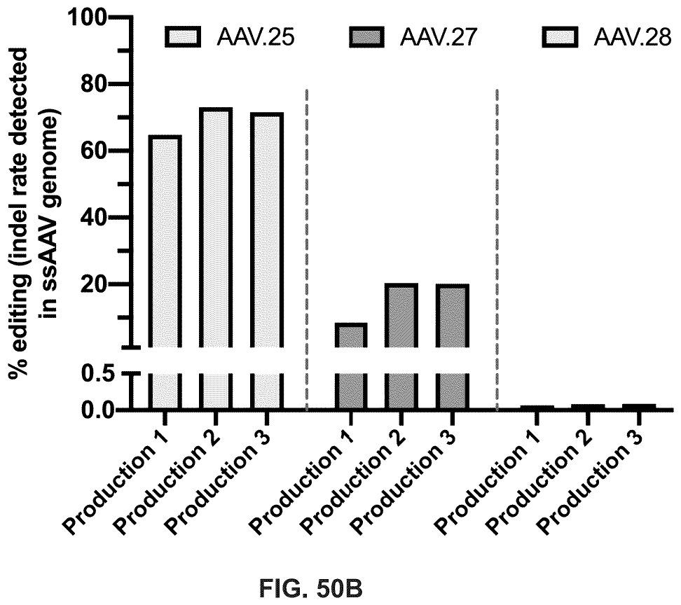

These results were confirmed by NGS analysis of ssDNA at the expected cleavage site in the transgene from the packaged AAV (FIG. 50A). Production plasmids were used as controls and showed no editing at the self-limiting segment site. Levels of indels found in ssDNA from packaged siAAV correlated with the decreases in titers observed; AAV.24 had nearly 100% edited ssDNA, compared to AAV.27 (˜20%). These results were consistent across different production batches and AAV fractions (FIG. 50B).

Experiments resulting the data in FIG. 113 further investigated the effects of using additional alternative PAMs beyond the four NTCN PAMs; the PAMs tested in FIG. 113 were TTC (construct 148), ATC (construct 149), CTC (construct 150), GTC (construct 151), TTT (construct 152), GTT (construct 153), CTT (construct 154), ATT (construct 155), TGT (construct 156), and CGC (construct 157). NGS analysis of the ssDNA packaged in the siAAVs expressing CasX (siAAVs) with STALL sites using the various alternative PAMs revealed that siAAV constructs 148 to 151, which contained the NTCN PAMs, resulted in a higher cleavage rate (and thus lower percentage of intact AAV genomes) of ˜50% to ˜70% (FIG. 113). However, siAAV construct 152 (containing a TTT PAM) resulted in ˜20% cleavage rate. For siAAV constructs 153 to 157, only use of siAAV construct 153 resulted in a true cleavage rate of the AAV genomes (˜2%), since a closer analysis of the indel profile for each siAAV sample revealed a minor background contamination that contributed to an apparent cleavage rate for constructs 154 to 157 (FIG. 113). As anticipated, with the NO STALL experimental control construct 158, at least ˜98% of the AAV genomes remained intact (FIG. 113).

The results confirm that self-inactivating AAV constructs can be designed to affect, in a temporal and quantitative sense, cleavage of the AAV plasmid by taking advantage of the variety of PAM sequences and their “strength” within the CasX system. Such systems are expected to ameliorate safety concerns relating to off-target editing or an immune response triggered by prolonged expression of non-human proteins delivered in traditional AAV systems.

### Example 13: Self-Inactivating Efficiency can be Regulated by Using Alternative Regulatory Elements Such as RNA Pol III Promoter

This experiment demonstrates that the potency of small CRISPR proteins (such as CasX) encoded in AAV constructs can be modulated if certain RNA promoters are chosen for expression of the guide RNA incorporated in the construct. Differences in promoter strength can be exploited to create siAAV-CRISPR systems as follows. By using RNA promoters with different strengths, constructs can be designed that modulate guide RNA expression, which affects editing potency. As the siAAV system provides enough cargo space in the transgene to contain at least 2 independent RNA promoters expressing 2 guide RNAs, multiple guide RNAs under the control of different promoters can be “tuned” within a single AAV transgene. For siAAVs, this can be used to control the expression of a dedicated gRNA for self-inactivation. By identifying RNA promoters of differing strengths, constructs can be designed with pairs of RNA promoters that yield the ideal timing between the therapeutic and inactivating editing outcomes. A schematic of an exemplary vector design used in this siAAV system is presented as FIG. 47. Additionally, RNA promoters were tested that are inverted versions of each other to confirm that these RNA promoters can be used bidirectionally to promote transcription.

### Materials and Methods

AAV vectors were generated and produced as described in Example 1. 5′ sequences used to generate the AAV plasmid contained enhancer, CasX protein promoter and N-terminal NLS, while 3′ sequences contained C-terminal NLS, WPRE, poly(A) signal, RNA promoter (Table 25) and guide RNA containing spacer 12.7.

TABLE 25
RNA promoter sequences used to drive expression of
the sgRNA guide incorporated in the pAAV transgene.

| siAAV construct ID | RNA promoter | Size (bp) | SEQ ID NO |
|---|---|---|---|
| 31/30 | hU6 isoform 1 | 241 | 661 |
| 72 | H1 | 215 | 662 |
| 73 | 7SK | 267 | 496 |
| 54/58 | hU6 variant 1 | 103 | 497 |
| 55 | hU6 variant 2 | 38 | 498 |
| 56 | hU6 variant 3 | 67 | 499 |
| 57 | hU6 variant 4 | 79 | 500 |
| 59 | hU6 variant 5 | 111 | 501 |
| 60 | hU6 variant 6 | 127 | 502 |
| 61 | hU6 variant 7 | 123 | 503 |
| 62 | hU6 variant 8 | 143 | 504 |
| 63 | hU6 variant 9 | 131 | 505 |
| 64 | hU6 variant 10 | 159 | 506 |
| 65 | hU6 variant 11 | 103 | 507 |
| 66 | hU6 variant 12 | 111 | 508 |
| 67 | hU6 variant 13 | 127 | 509 |
| 68 | hU6 variant 14 | 103 | 510 |
| 69 | hU6 variant 15 | 131 | 511 |
| 70 | hU6 variant 16 | 159 | 512 |
| 71 | hU6 variant 17 | 128 | 513 |
| — | H1 core | 91 | 2688 |
| — | H1 core + 7SK hybrid 1 | 92 | 2689 |
| — | H1 core + 7SK hybrid 2 | 92 | 2690 |
| — | H1 core + 7SK hybrid 3 | 91 | 2691 |
| — | H1 core + 7SK hybrid 4 | 91 | 2692 |
| — | H1 core + 7SK hybrid 5 | 92 | 2693 |
| — | H1 core + 7SK hybrid 6 | 91 | 2694 |
| — | H1 core + 7SK hybrid 7 | 91 | 2695 |
| — | H1 core + 7SK hybrid 8 | 91 | 2696 |
| — | H1 core + 7SK hybrid 9 | 92 | 2697 |
| — | H1 core + U6 hybrid 1 | 91 | 2698 |
| — | H1 core + U6 hybrid 2 | 94 | 2699 |
| — | H1 core + 7SK + U6 | 92 | 2700 |
| | hybrid 1 | | |
| — | H1 core + U6 hybrid 3 | 90 | 2701 |
| — | H1 core + 7SK + U6 | 94 | 2702 |
| | hybrid 2 | | |
| — | H1 core + 7SK + U6 | 94 | 2703 |
| | hybrid 3 | | |
| — | hU6 isoform 2 | 247 | 2704 |
| — | hU6 isoform 3 | 249 | 2705 |
| — | hU6 isoform 4 | 249 | 2706 |
| — | hU6 isoform 5 | 249 | 2707 |
| 97-103, 110-116 | mU6 | 304 | 2708 |

mNPC nucleofection, AAV production, transduction, and FACS analysis were conducted as described in Example 1.

### Results:

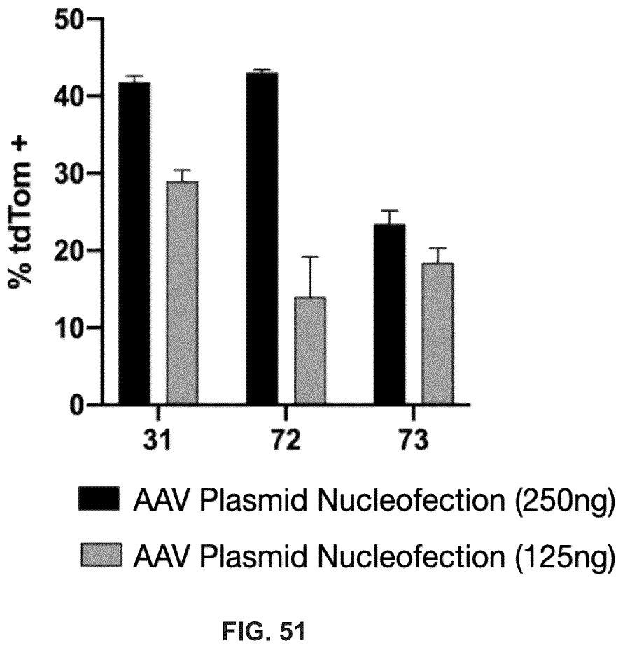

The results shown in FIG. 51 demonstrate variable editing with three distinct RNA promoters with CasX 491, scaffold variant 174 and spacer 12.7. When AAV transgene plasmids were nucleofected into mNPCs at doses of 250 ng and 125 ng, constructs 72 and 73 show ˜40% of the activity of the base construct 31 at the 125 ng dose. At the 250 ng dose, construct 72 shows similar editing to the base construct 31, and construct 73 shows ˜50% of editing with respect to construct 31.

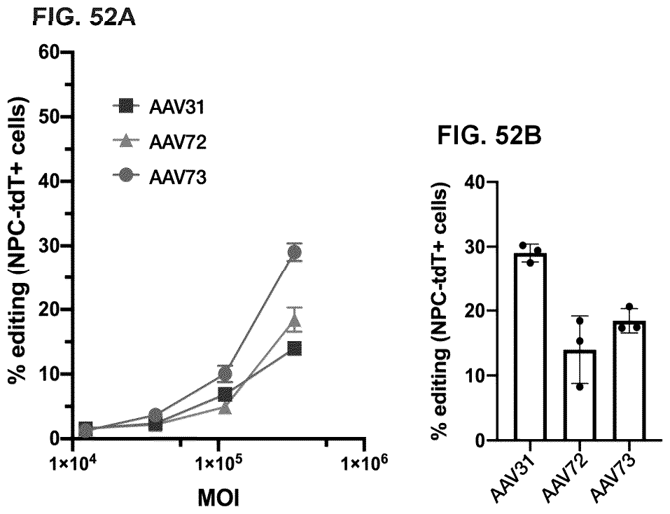

The results in FIGS. 52A-52B demonstrate that the same three distinct RNA promoters with protein 491, scaffold variant 174 and spacer 12.7, when delivered as AAV, edited the target stop cassette in mNPCs. AAV.3, AAV.32, AAV.33 were generated with transgene constructs 3, 32 and 33 respectively. Each vector displayed dose-dependent editing at the target locus (FIG. 52A). At an MOI of 3E5, AAV.32 and AAV.33 had 50-60% of the potency of AAV.3 (FIG. 52B).

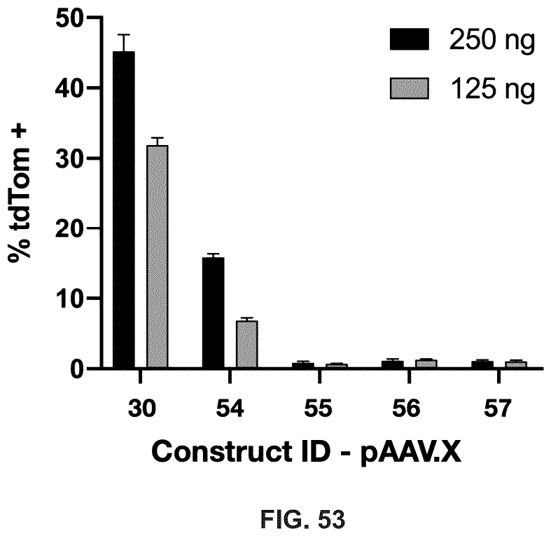

The results shown in FIG. 53 demonstrate that four truncations of an RNA promoter with protein 491, scaffold variant 174 and spacer 12.7, when delivered by nucleofection of AAV transgene plasmid, edited the target stop cassette at differential levels in mNPCs at doses of 250 ng and 125 ng. Construct 54 had 33% of the potency of the base construct 30, while constructs 55, 56 and 57 did not show any editing and were comparable to a non-targeting control.

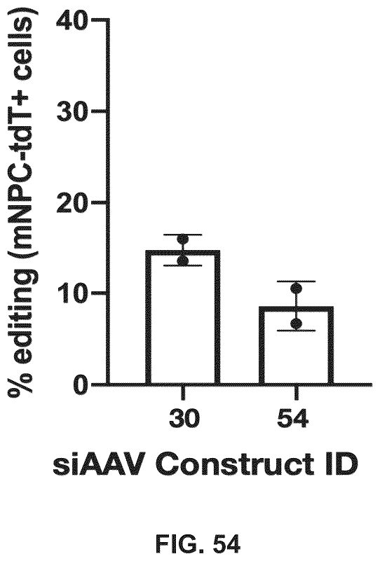

The results shown in FIG. 54 compared editing in mNPCs between base construct 30 to construct 54 when delivered as AAV. AAV.54 was able to edit at 7% compared to 15% for AAV.30 at an MOI of 3E5, consistent with the results from FIG. 54.

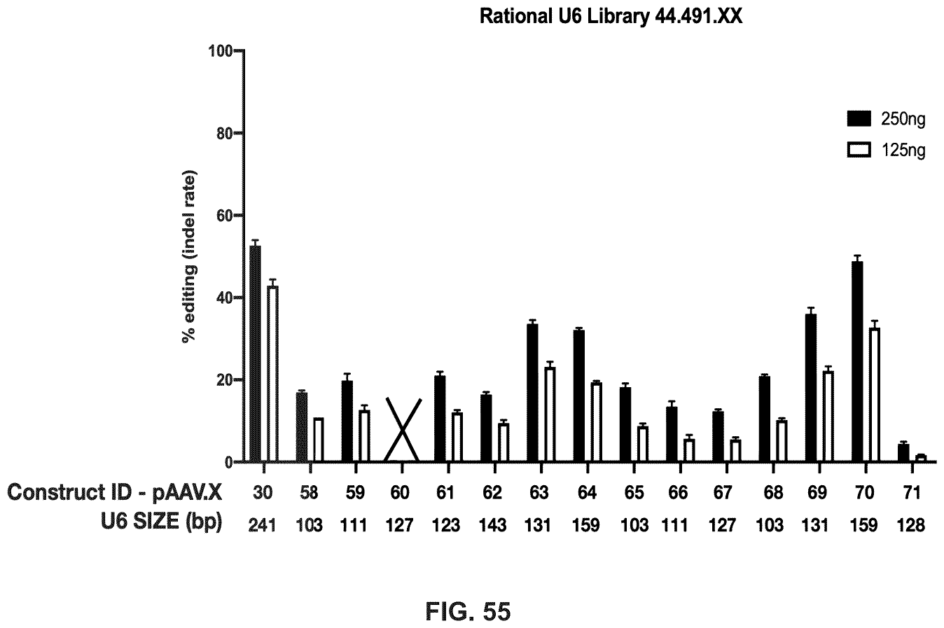

The results shown in FIG. 55 demonstrate that engineered RNA promoters with protein 491, scaffold variant 174 and spacer 12.7, when delivered by nucleofection of AAV transgene plasmid, edited the target stop cassette at differential levels in mNPCs at doses of 250 ng and 125 ng. One cluster of constructs (58, 59, 61, 62, 65, 66, 67, 68) all edit 15-20% compared to 55% for construct 30. Other Pol III variants, i.e., constructs 63, 64 and 69, all exhibited higher levels of editing at around 32% editing while construct 70 displayed 48% editing. These promoters are all smaller than the Pol III promoter in the base construct 30.

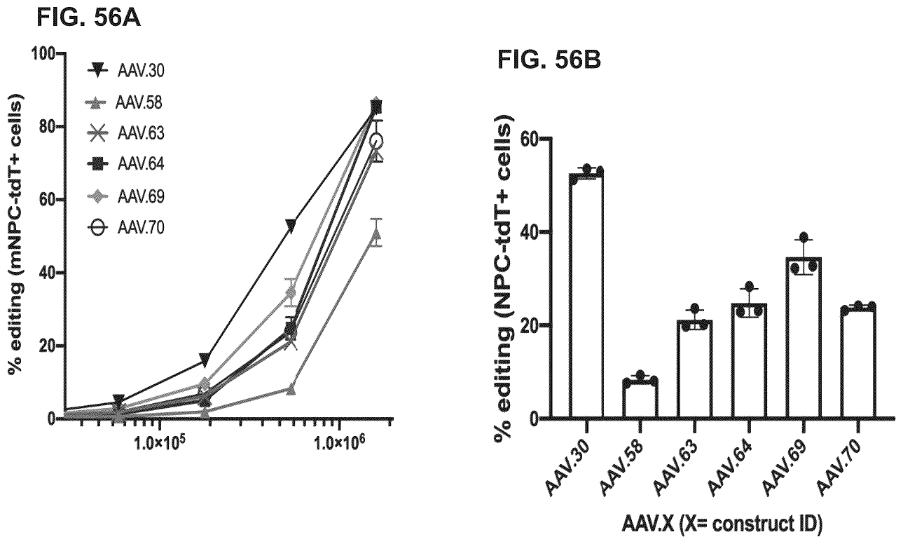

The results shown in FIGS. 56A and 56B demonstrate that engineered RNA promoters with protein 491, scaffold variant 174 and spacer 12.7, when delivered as AAV, edited the target stop cassette in mNPCs. Variable rates of editing with AAV were seen with constructs AAV.63, AAV.64, AAV.69, and AAV.70; all editing at rates between the base constructs AAV.30 and AAV.58.

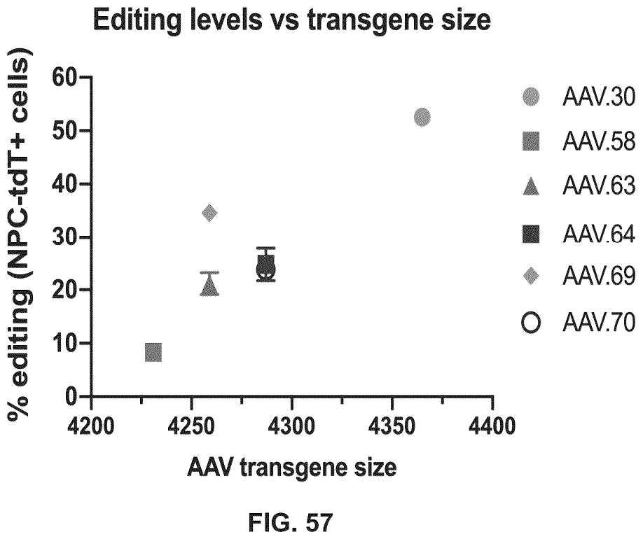

The scatterplot shown in FIG. 57 shows editing as a function of the size of the total transgene size of the AAV variants. Constructs AAV.63, AAV.64, AAV.69, and AAV.70 all are shorter than the base U6 promoter in AAV.30 (see Table 25). This enables a relaxing of size constraints for other components in AAV designs.

The results of these experiments show that when a second promoter and guide are utilized for the self-inactivating siAAV system, a promoter can be selected with a differing strength than the one transcribing the therapeutic guide targeting the host gene to be edited. This is relevant for strategies where the timing of self-inactivation relative to the time required to achieve host gene editing is critical; i.e., a delay in self-inactivation relative to editing of the target nucleic acid is desired.

These experiments also show siAAV can be designed with pairs of RNA promoters that can function in both orientations (AAV.94 and AAV.100, AAV.95 and AAV.101). This indicates that it is possible to express two guides from a single RNA promoter, modulating the strength of transcription initiation simply by positioning guides in forward or reverse orientation relative to the promoter. This approach can be used in siAAV constructs by placing the therapeutic guide in the stronger direction and the self-inactivating guide in the weaker direction. Such constructs also conserve cargo space for inclusion of other elements in the transgene.

### Example 14: Self-Inactivating Efficiency in siAAV can be Regulated by Using Alternative sgRNA-Scaffold Sequences

In order to progressively decrease expression of the CRISPR system nuclease after achieving the desirable editing outcome, constructs of the siAAV system were designed to assess the ability of the self-inactivating segments to be modulated by different “weaker” gRNA-guide variants compared to the gRNA incorporated to edit the target nucleic acid. Addition of a second gRNA guide variant targeting a self-inactivating sequence incorporated in the transgene has many advantages, such as the ability to target any sequences or elements incorporated or inherently occurring in the siAAV transgene. The purpose of these experiments was to show that the use of a weaker scaffold targeted to the self-inactivating segment would result in delayed cleavage with respect to editing of the genomic target that is targeted by a stronger guide scaffold and, therefore, longer durations of CasX expression prior to self-cleavage.

### Materials and Methods

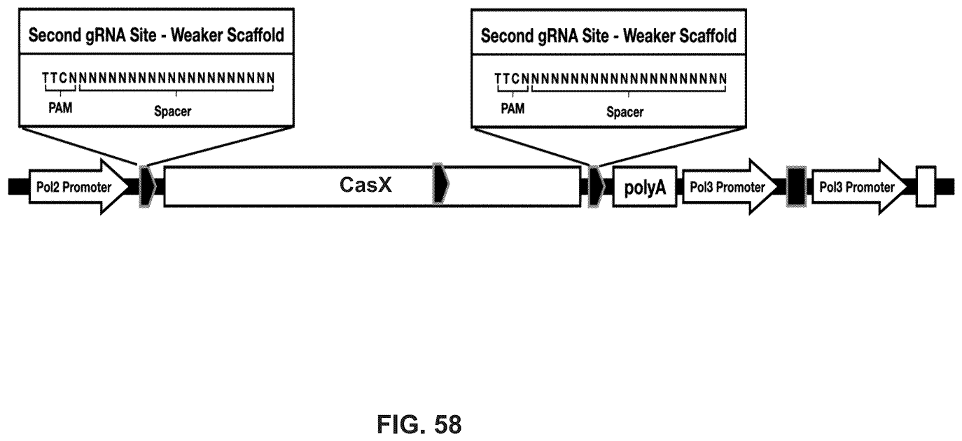

Guide scaffold variants were inserted into an siAAV transgene construct for plasmid and viral vector validation. Representative schematics of the designs are shown in FIG. 46 and FIG. 58. The AAV transgene between the ITRs was conceptually broken up into different parts, which consisted of sequences encoding the nuclease and accessory elements relevant to expression in mammalian cells and the guide scaffold and targeting spacer. The spacer 12.7 and guide sequences utilized in single-guide constructs are listed in Table 26. In constructs with dual guides, the second guide was made with spacer 12.2 (TATAGCATACATTATACGAA, SEQ ID NO: 536) or was non-targeting (CTGCATTCTAGTTGTGGTTT, SEQ ID NO: 462).

For these experiments, siAAV vectors were generated using the previously described methods for AAV production and purification, and nucleofection and editing assays were conducted as described in Examples 1 and 12.

TABLE 26
Guide sequences cloned into p59.491.U6.X.Y plasmids

| | Guide | | | | | | |
|---|---|---|---|---|---|---|---|
| Construct | spacer | Spacer | SEQ | Guide | SEQ | | SEQ |
| ID | variants | Sequence | ID NO | Sequence | ID NO | Guide + Spacer Sequence | ID NO |
| 31 | 174.12.7 | CTGCATT | 462 | ACTGGCGCTTTT | 691 | ACTGGCGCTTTTATCTGA | 701 |
| | | CTAGTTG | | ATCTgATTACTT | | TTACTTTGAGAGCCATCA | |
| | | TGGTTT | | TGAGAGCCATCA | | CCAGCGACTATGTCGTAG | |
| | | | | CCAGCGACTATG | | TGGGTAAAGCTCCCTCTT | |
| | | | | TCGTAgTGGGTA | | CGGAGGGAGCATCAAAGC | |
| | | | | AAGCTCCCTCTT | | TGCATTCTAGTTGTGGTT | |
| | | | | CGGAGGGAGCAT | | T | |
| | | | | CAAAG | | | |
| 39 | 229.12.7 | CTGCATT | 682 | ACTGGCACTTTT | 692 | ACTGGCACTTTTATCTGA | 702 |
| | | CTAGTTG | | ATCTGATTACTT | | TTACTTTGAGAGCCATCA | |
| | | TGGTT | | TGAGAGCCATCA | | CCAGCGACTATGTCGTAT | |
| | | | | CCAGCGACTATG | | GGGTAAAGCGCTTACGGA | |
| | | | | TCGTATGGGTAA | | CTTCGGTCCGTAAGAAGC | |
| | | | | AGCGCTTACGGA | | ATCAAAGCTGCATTCTAG | |
| | | | | CTTCGGTCCGTA | | TTGTGGTTT | |
| | | | | AGAAGCATCAAA | | | |
| | | | | G | | | |
| 40 | 230.12.7 | CTGCATT | 683 | ACTGGCACTTCT | 693 | ACTGGCACTTCTATCTGA | 703 |
| | | CTAGTTG | | ATCTGATTACTC | | TTACTCTGAGAGCCATCA | |
| | | TGGTT | | TGAGAGCCATCA | | CCAGCGACTATGTCGTAT | |
| | | | | CCAGCGACTATG | | GGGTAAAGCGCTTACGGA | |
| | | | | TCGTATGGGTAA | | CTTCGGTCCGTAAGAAGC | |
| | | | | AGCGCTTACGGA | | ATCAGAGCTGCATTCTAG | |
| | | | | CTTCGGTCCGTA | | TTGTGGTTT | |
| | | | | AGAAGCATCAGA | | | |
| 41 | 231.12.7 | CTGCATT | 684 | ACTGGCGCTTCT | 694 | ACTGGCGCTTCTATCTGA | 704 |
| | | CTAGTTG | | ATCTGATTACTC | | TTACTCTGAGAGCCATCA | |
| | | TGGTT | | TGAGAGCCATCA | | CCAGCGACTATGTCGTAT | |
| | | | | CCAGCGACTATG | | GGGTAAAGCCGCTTACGG | |
| | | | | TCGTATGGGTAA | | ACTTCGGTCCGTAAGAGG | |
| | | | | AGCCGCTTACGG | | CATCAGAGCTGCATTCTA | |
| | | | | ACTTCGGTCCGT | | GTTGTGGTTT | |
| | | | | AAGAGGCATCAG | | | |
| | | | | AG | | | |
| 42 | 232.12.7 | CTGCATT | 685 | ACTGGCACTTCT | 695 | ACTGGCACTTCTATCTGA | 705 |
| | | CTAGTTG | | ATCTGATTACTC | | TTACTCTGAGCGCCATCA | |
| | | TGGTT | | TGAGCGCCATCA | | CCAGCGACTATGTCGTAT | |
| | | | | CCAGCGACTATG | | GGGTAAAGCCGCTTACGG | |
| | | | | TCGTATGGGTAA | | ACTTCGGTCCGTAAGAGG | |
| | | | | AGCCGCTTACGG | | CATCAGAGCTGCATTCTA | |
| | | | | ACTTCGGTCCGT | | GTTGTGGTTT | |
| | | | | AAGAGGCATCAG | | | |
| | | | | AG | | | |
| 43 | 233.12.7 | CTGCATT | 686 | ACTGGCGCTTCT | 696 | ACTGGCGCTTCTATCTGA | 706 |
| | | CTAGTTG | | ATCTGATTACTC | | TTACTCTGAGCGCCATCA | |
| | | TGGTT | | TGAGCGCCATCA | | CCAGCGACTATGTCGTAT | |
| | | | | CCAGCGACTATG | | GGGTAAAGCCGCTTACGG | |
| | | | | TCGTATGGGTAA | | ACTTCGGTCCGTAAGAGG | |
| | | | | AGCCGCTTACGG | | CATCAGAGCTGCATTCTA | |
| | | | | ACTTCGGTCCGT | | GTTGTGGTTT | |
| | | | | AAGAGGCATCAG | | | |
| | | | | AG | | | |
| 44 | 234.12.7 | CTGCATT | 687 | ACTGGCGCTTCT | 697 | ACTGGCGCTTCTATCTGA | 707 |
| | | CTAGTTG | | ATCTGATTACTC | | TTACTCTGAGCGCCATCA | |
| | | TGGTT | | TGAGCGCCATCA | | CCAGCGACTATGTCGTAT | |
| | | | | CCAGCGACTATG | | GGGTAAAGCGCCTTACGG | |
| | | | | TCGTATGGGTAA | | ACTTCGGTCCGTAAGGAG | |
| | | | | AGCGCCTTACGG | | CATCAGAGCTGCATTCTA | |
| | | | | ACTTCGGTCCGT | | GTTGTGGTTT | |
| | | | | AAGGAGCATCAG | | | |
| | | | | AG | | | |
| 45 | 235.12.7 | CTGCATT | 688 | ACTGGCGCTTCT | 698 | ACTGGCGCTTCTATCTGA | 708 |
| | | CTAGTTG | | ATCTGATTACTC | | TTACTCTGAGCGCCATCA | |
| | | TGGTT | | TGAGCGCCATCA | | CCAGCGACTATGTCGTAG | |
| | | | | CCAGCGACTATG | | TGGGTAAAGCCGCTTACG | |
| | | | | TCGTAGTGGGTA | | GACTTCGGTCCGTAAGAG | |
| | | | | AAGCCGCTTACG | | GCATCAGAGCTGCATTCT | |
| | | | | GACTTCGGTCCG | | AGTTGTGGTT | |
| | | | | TAAGAGGCATCA | | | |
| | | | | GAG | | | |
| 46 | 236.12.7 | CTGCATT | 689 | ACGGGACTTTCT | 699 | ACGGGACTTTCTATCTGA | 709 |
| | | CTAGTTG | | ATCTGATTACTC | | TTACTCTGAAGTCCCTCA | |
| | | TGGTT | | TGAAGTCCCTCA | | CCAGCGACTATGTCGTAT | |
| | | | | CCAGCGACTATG | | GGGTAAAGCCGCTTACGG | |
| | | | | TCGTATGGGTAA | | ACTTCGGTCCGTAAGAGG | |
| | | | | AGCCGCTTACGG | | CATCAGAGCTGCATTCTA | |
| | | | | ACTTCGGTCCGT | | GTTGTGGTT | |
| | | | | AAGAGGCATCAG | | | |
| | | | | AG | | | |
| 47 | 237.12.7 | CTGCATT | 690 | ACCTGTAGTTCT | 700 | ACCTGTAGTTCTATCTGA | 710 |
| | | CTAGTTG | | ATCTGATTACTC | | TTACTCTGACTACAGTCA | |
| | | TGGTT | | TGACTACAGTCA | | CCAGCGACTATGTCGTAT | |
| | | | | CCAGCGACTATG | | GGGTAAAGCCGCTTACGG | |
| | | | | TCGTATGGGTAA | | ACTTCGGTCCGTAAGAGG | |
| | | | | AGCCGCTTACGG | | CATCAGAGCTGCATTCTA | |
| | | | | ACTTCGGTCCGT | | GTTGTGGTT | |
| | | | | AAGAGGCATCAG | | | |
| | | | | AG | | | |

### Results:

A self-inactivating AAV-CRISPR system (siAAV) that incorporated a second specialized guide in the AAV dedicated to self-cleavage was designed. This dual guide system allowed for a “weaker” guide scaffold to be linked to the self-targeting spacer (black pointed symbol in FIG. 58 indicates the target sequence of the self-targeting spacer) inserted in different locations of the transgene and, therefore, delay self-cleavage relative to the editing of the genomic target mediated by the nuclease and the “stronger” guide scaffold (white pointed symbol in FIG. 58) resulting in longer durations of CasX expression prior to self-cleavage.

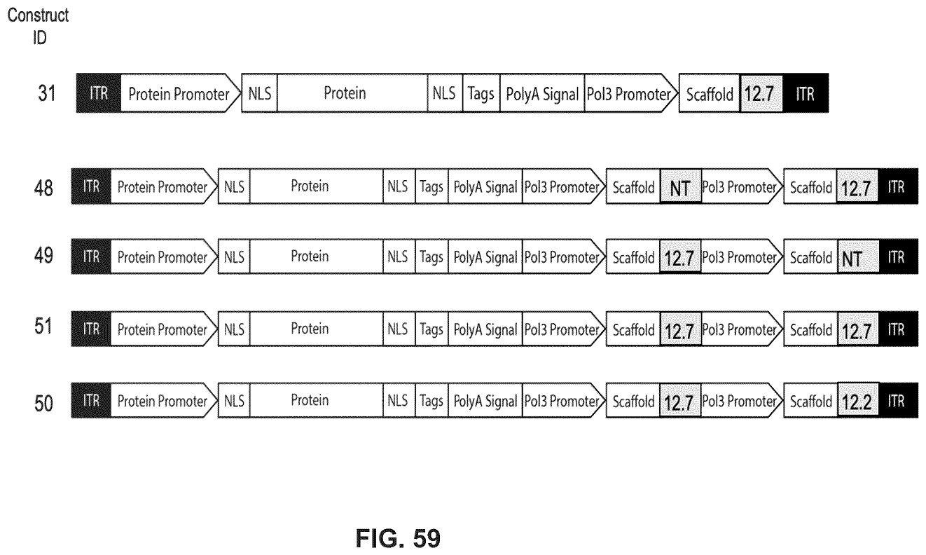

FIG. 59 (top) shows the design of single guide targeting construct 31 and dual gRNA vectors 48, 49, 50, 51 based on sgRNA 174. Constructs 48 and 49 harbored one scrambled spacer (non-targeting, NT).

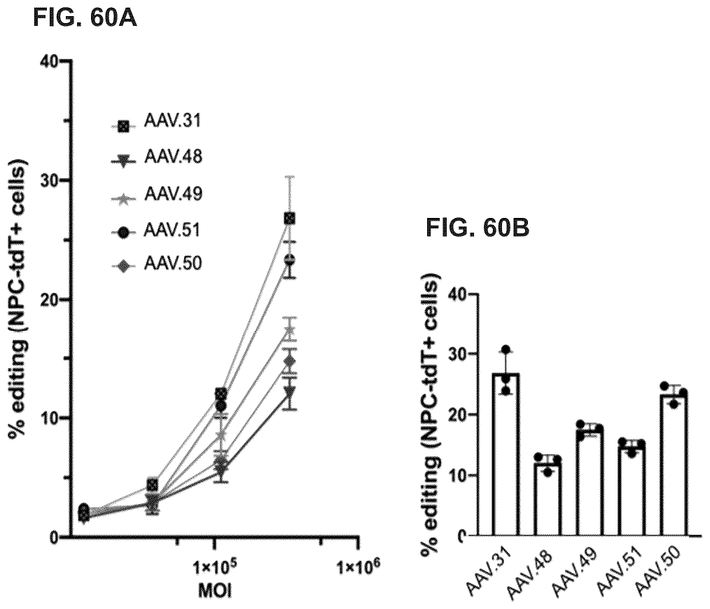

The results of FIGS. 60A and B show that AAV.31 and AAV.50 have similar editing levels (˜28% vs 25% at 3.0E+5 vg/cell MOI), confirming that the dual gRNA guide system efficiently edits. Furthermore, the 2nd sgRNA edited the genomic target in mNPCs at lower efficiency than the 1st sgRNA (AAV.48 vs AAV.49 respectively), which indicated that modulation of editing levels can be mediated by choice of the gRNA guide position in the dual guide system.

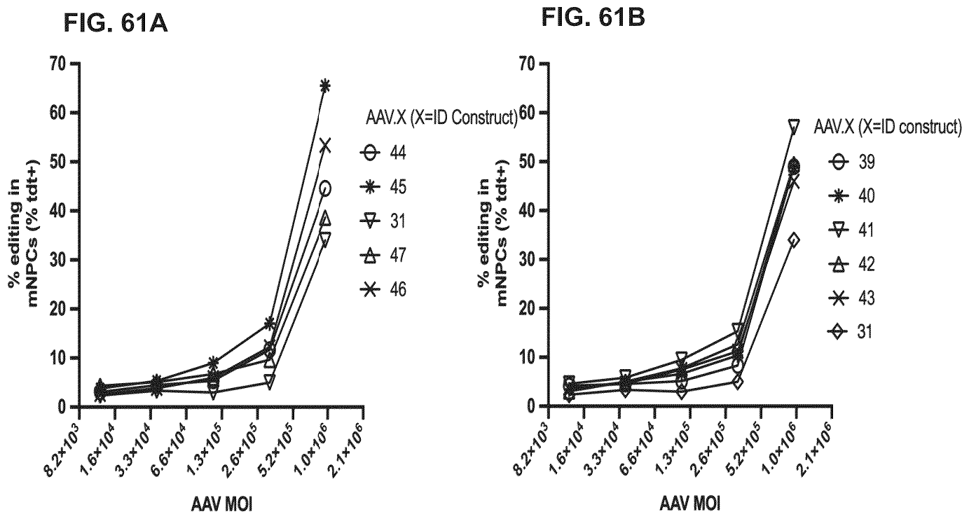

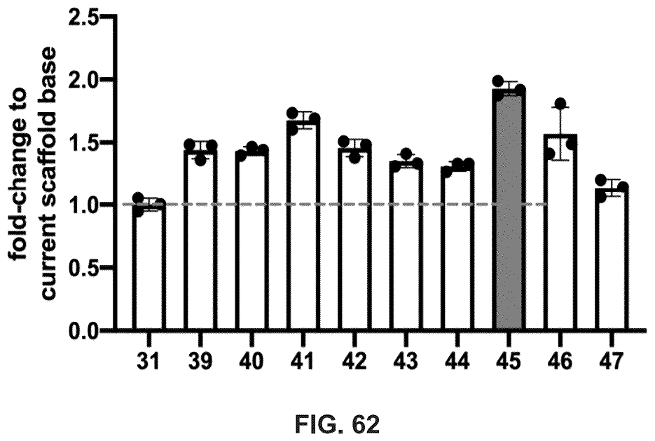

Modulation of activity with the dual gRNA guide system can also be further engineered by using different gRNA scaffold variants. Table 26 lists the encoding sequences of the gRNA scaffold variants packaged into siAAV and tested in vitro in mNPC-tdT cells. Vectors expressing scaffold variants 231-236 (AAV.41-46) performed at higher levels than ones with scaffold 174 and 237 (AAV.31/47) respectively (FIGS. 61A and B). AAV constructs with gRNA scaffold 235 displayed a 2-fold increased activity relative to constructs with gRNA scaffold 174 at a MOI of 3.0e+5 (AAV.45 vs AAV.31; FIG. 62). AAV constructs with a variety of scaffold variants displayed a 1.5-fold increase relative to scaffold 174 (AAV.41-44 vs. AAV.31).

AAV expressing dual gRNA guide stacks is an efficient system to allow multiple genomic sequences to be edited within the same AAV vector, and therefore are very useful for self-cleaving AAV systems. The results support that editing efficiency can be precisely modulated by the use of engineered gRNA guide variants with scaffold-mediated different levels of activity.

### Example 15: siAAVs Ensure Efficient AAV Episomal Removal

AAVs are efficient delivery vehicles for gene therapies. However, the stability of their episomal genomes allows for their persistent expression in the nucleus, which over a long period of time can trigger potential off-targeting effects and undesired immunogenic responses in the transduced cells. Designing a self-inactivating AAV-CRISPR/CasX system to restrict the persistent expression of delivered therapeutic AAVs mitigates these undesired consequences after the target genomic locus has been edited.

To develop a self-inactivating AAV-CRISPR/CasX system, a strategy leveraging the differential Protospacer Adjacent Motif (PAM) recognition capability of CasX to mediate its variable editing levels was employed. It was previously shown that CasX has the strongest relative preference for the TTC PAM (thus resulting in the highest cleavage rate), followed by the ATC PAM, and then the CTC PAM. Here, the nuclease construct was engineered such that the coding sequence for CasX was flanked by proto-spacer sites identical to the on-target tdTomato proto-spacer site in the mouse genome (hereafter tdTom proto-spacer), in addition to the targeting gRNA bearing the tdTomato spacer sequence. The flanking tdTom proto-spacer sequences were preceded by TTC, ATC, or CTC PAMs, while the TTC PAM was present at the tdTomato proto-spacer site in the mouse genome. The presence of PAMs with reduced recognition efficiency at the flanking tdTom proto-spacer sites allows CasX to be expressed for sufficient duration to achieve a concentration to cut the high efficiency PAM tdTom proto-spacer at the genomic target locus before eventually targeting its own coding sequence for self-inactivation. As a negative control, a nuclease construct without the flanking tdTom protospacer sequences for self-inactivation was used.

### Materials and Methods

To evaluate the self-inactivation activity of the designed siAAV system, AAVs containing CasX-tdTomato gRNA genes, with or without the self-inactivation system, were produced by co-transfecting the three plasmids (an adenoviral helper plasmid, an AAV packaging plasmid, and the AAV transgene) into LentiX HEK293T cells, as previously described in Example 12. Results:

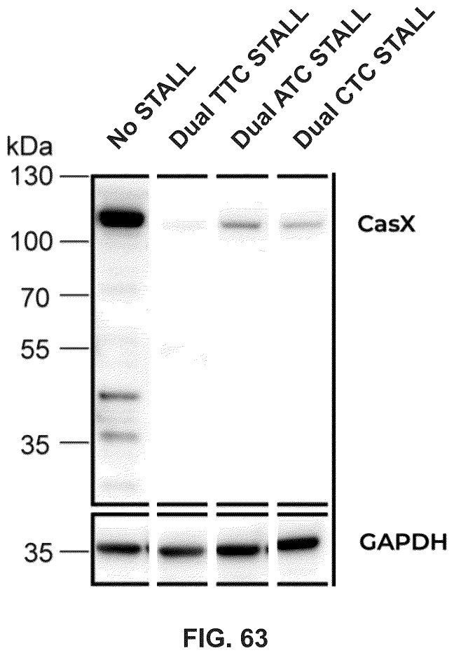

FIG. 63 is a western blot analysis of CasX protein levels in transfected HEK293T cells, with GAPDH serving as the loading control. Cells transfected with the AAV-CRISPR construct without the flanking tdTom protospacer sequences for self-inactivation are labeled as ‘No STALL’ (lane 1), while cells transfected with the constructs harboring the flanking tdTom protospacer sequences preceded by TTC, ATC, or CTC PAMs are labelled as Dual TTC STALL, Dual ATC STALL, or Dual CTC STALL respectively (lanes 2-4). CasX protein levels for the Dual STALL variants were quantified from the western blot. The Dual TTC STALL construct resulted in a 238-fold reduction in CasX protein levels, while the Dual ATC STALL resulted in a 27-fold reduction and the Dual CTC STALL resulted in a 38-fold reduction relative to the CasX levels with the No STALL control construct.

The results show that AAVs harboring the tdTom protospacer sequences with TTC PAM, for which CasX 491 has the highest relative affinity, demonstrated the strongest self-inactivating activity as illustrated by the highest fold knockdown of CasX protein levels. Meanwhile, AAVs expressing constructs that result in lower CasX-PAM binding affinity (Dual ATC STALL and Dual CTC STALL) displayed weaker self-inactivating activity and therefore, lower relative knockdown of CasX. These data indicate that the level of CasX self-cleavage can be effectively modulated by varying PAMs with reduced recognition efficiency at the protospacer sequences flanking the nuclease construct. More importantly, such fine tuning of CasX activity and self-inactivation allows optimization of the therapeutic editing capability of CasX while minimizing its potential to induce off-target effects and undesired immunogenic outcomes.

### Example 16: siAAVs do not Impede CRISPR Editing Levels

This experiment assessed levels of on-target editing generated by siAAVs compared to an AAV that did not contain a self-inactivating segment.

### Materials and Methods

AAV vector production: Viral vectors were produced as described in Example 12. The self-inactivating sequence targeted by spacer 12.7 was incorporated in the AAV transgene at the junction between the promoter and the Kozak sequence with 5 different PAM sequences (TTCA, CTC, ATC, GTC and GGGG, in AAV.24, 25, 26, 27, 28 respectively). Vector AAV.31 does not contain a self-targeting sequence.

Reporter cell lines: Primary mouse cortical tissue was isolated by dissection from embryonic 18.5 pups, dissociated (MACS papain dissociation kit) and plated in neurobasal media supplemented with B27, 25 μM glutamic Acid, and 1× penicillin/streptomycin. 20,000 cells were plated on PLF coated plates for AAV transduction analysis. AAV transduction and assessing editing activity by FACS was conducted as described in Example 12.

### Results:

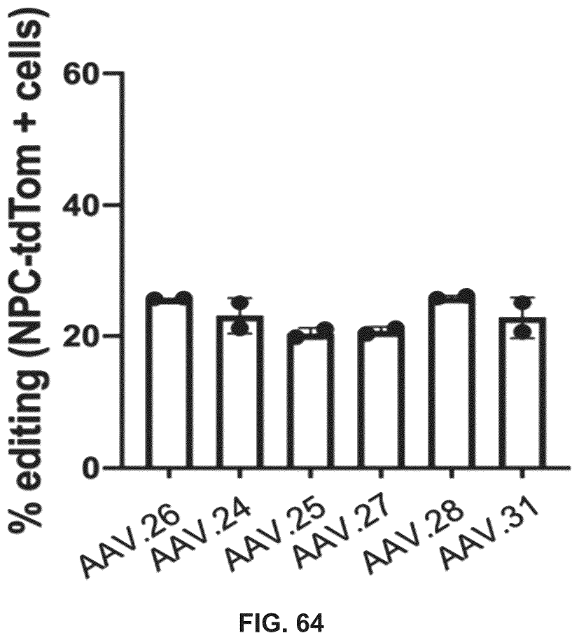

The results of the assay (FIG. 64) show the transduction levels in cortical neurons cultures derived from primary neuroprogenitor cells with six AAVs (AAV.24, 25, 26, 27, 28) harboring the self-inactivating spacer in transgene cassettes with different PAM sequences (TTC, CTC, ATC, GTC). Although the self-inactivating spacer impacted AAV titer, as shown in Example 12, based on editing strength mediated by the PAM (TTC>CTC>ATC>GTC), MOI-normalized siAAVs and control AAVs had similar editing levels (20-25% editing at 5.0e+5 vg/cell), indicated that editing of the target was not impacted by the self-inactivation.

These results shows that although the self-inactivating AAV-CRISPR systems efficiently led to transgene self-cleavage over time (see Example 12), the on-target editing levels in cells were not significantly impeded, supporting the utility of the siAAV system.

### Example 17: shRNAs Silence CRISPR Protein Expression in Production Cell Lines

The results of NGS analysis of packaged siAAV genomes in Example 12 showed that CasX cleaved the therapeutic transgene during production of the AAV in packaging cells, which leads to the undesirable outcome of the packaging of partial AAV genomes. A strategy to bypass the cleaving of the AAV transgene is to down-regulate the expression of CasX only during AAV production. One way to do this is through silencing translation of CasX using shRNAs by degrading the CasX mRNA transcript. The goal of this experiment was to demonstrate that shRNA can silence the CasX protein expression in AAV producing cells and prevent the packaging of partial AAV genomes during production. FIG. 72 shows the possible vector arrangements for supplying one or more shRNA during AAV packaging. The shRNA, or multiple shRNAs can either be supplied on the same plasmid as the AAV viral genome comprising the transgene, on another plasmid in production such as pRepCap or pHelper plasmid, on multiple production plasmids, or on a separate polynucleotide (FIG. 72).

### Materials and Methods

Cloning shRNA Sequences:

A destination vector allowing for easy cloning of multiple shRNA constructs was generated from p59.491.174.NT (containing the encoding sequences for CasX 491, gRNA 174 and a non-targeting spacer). p59 was digested with a PciI, which cuts between the bacterial origin and the AAV ITR. The shRNA destination site was created by performing PCR of an eGFP gene with an EF-1α promoter and ordering a sequence coding for a 3′ UTR containing multiple unique restriction sites and then inserting the fragments into the digested p59 vector with Gibson Assembly. shRNA sequences (Table 9 and Table 27) were designed with a miR-30a backbone to direct processing in the cell. To clone shRNAs into the first cloning site, the shRNA sequences were amplified with primers to create homology arms on either side of the AvrII cut site. The destination vector was digested with AvrII and the shRNA was inserted by Gibson Assembly. To make plasmids with two shRNAs, the single-shRNA plasmid was digested with NheI, the shRNA to be inserted into the second site was PCR-amplified with primers to add homology arms for the NheI site, and the two fragments were assembled with Gibson Assembly. This resulted in a plasmid in which the shRNA sequence, or sequences, were included on the same plasmid as the AAV transgene sequence, but were outside of the region between the ITRs that was packaged into the AAV vectors by the packaging cells.

TABLE 27
shRNA sequences

| | siAAV | | | | |
|---|---|---|---|---|---|
| | construct | | SEQ ID | | SEQ ID |
| Name | ID | DNA sequence | NO: | RNA sequence* | NO: |
| shRNA1a | 1 | CCTAGGCAACAGAAGGCT | 2861 | CCUAGGCAACAGAAGGCU | 2873 |
| | | AAAGAAGGTATATTGCTG | | AAAGAAGGUAUAUUGCUG | |
| | | TTGACAGTGAGCGACGCT | | UUGACAGUGAGCGACGCU | |
| | | GATCATCAATTACTTACT | | GAUCAUCAAUUACUUACU | |
| | | GTGAAGCCACAGATGGGT | | GUGAAGCCACAGAUGGGU | |
| | | AAGTAATTGATGATCAGC | | AAGUAAUUGAUGAUCAGC | |
| | | GCTGCCTACTGCCTCGGA | | GCUGCCUACUGCCUCGGA | |
| | | CTTCAAGGGGCTACTTTA | | CUUCAAGGGGCUACUUUA | |
| | | GGAGCAATTATC | | GGAGCAAUUAUC | |
| shRNA2a | 2 | CCTAGGCAACAGAAGGCT | 2862 | CCUAGGCAACAGAAGGCU | 2874 |
| | | AAAGAAGGTATATTGCTG | | AAAGAAGGUAUAUUGCUG | |
| | | TTGACAGTGAGCGACGCA | | UUGACAGUGAGCGACGCA | |
| | | ACTGCGCCTTCATTTACT | | ACUGCGCCUUCAUUUACU | |
| | | GTGAAGCCACAGATGGGT | | GUGAAGCCACAGAUGGGU | |
| | | AAATGAAGGCGCAGTTGC | | AAAUGAAGGCGCAGUUGC | |
| | | GCTGCCTACTGCCTCGGA | | GCUGCCUACUGCCUCGGA | |
| | | CTTCAAGGGGCTACTTTA | | CUUCAAGGGGCUACUUUA | |
| | | GGAGCAATTATC | | GGAGCAAUUAUC | |
| shRNA3a | 3 | CCTAGGCAACAGAAGGCT | 2863 | CCUAGGCAACAGAAGGCU | 2875 |
| | | AAAGAAGGTATATTGCTG | | AAAGAAGGUAUAUUGCUG | |
| | | TTGACAGTGAGCGACGCT | | UUGACAGUGAGCGACGCU | |
| | | GAGCAAGCACATTAAACT | | GAGCAAGCACAUUAAACU | |
| | | GTGAAGCCACAGATGGGT | | GUGAAGCCACAGAUGGGU | |
| | | TTAATGTGCTTGCTCAGC | | UUAAUGUGCUUGCUCAGC | |
| | | GCTGCCTACTGCCTCGGA | | GCUGCCUACUGCCUCGGA | |
| | | CTTCAAGGGGCTACTTTA | | CUUCAAGGGGCUACUUUA | |
| | | GGAGCAATTATC | | GGAGCAAUUAUC | |
| shRNA4a | 4 | CCTAGGCAACAGAAGGCT | 2864 | CCUAGGCAACAGAAGGCU | 2876 |
| | | AAAGAAGGTATATTGCTG | | AAAGAAGGUAUAUUGCUG | |
| | | TTGACAGTGAGCGACGCC | | UUGACAGUGAGCGACGCC | |
| | | TGATCATCAATTACTACT | | UGAUCAUCAAUUACUACU | |
| | | GTGAAGCCACAGATGGGT | | GUGAAGCCACAGAUGGGU | |
| | | AGTAATTGATGATCAGGC | | AGUAAUUGAUGAUCAGGC | |
| | | GCTGCCTACTGCCTCGGA | | GCUGCCUACUGCCUCGGA | |
| | | CTTCAAGGGGCTACTTTA | | CUUCAAGGGGCUACUUUA | |
| | | GGAGCAATTATC | | GGAGCAAUUAUC | |
| shRNA5a | 5 | CCTAGGCAACAGAAGGCT | 2865 | CCUAGGCAACAGAAGGCU | 2877 |
| | | AAAGAAGGTATATTGCTG | | AAAGAAGGUAUAUUGCUG | |
| | | TTGACAGTGAGCGACGGT | | UUGACAGUGAGCGACGGU | |
| | | ACCTGATCATCAATTACT | | ACCUGAUCAUCAAUUACU | |
| | | GTGAAGCCACAGATGGGT | | GUGAAGCCACAGAUGGGU | |
| | | AATTGATGATCAGGTACC | | AAUUGAUGAUCAGGUACC | |
| | | GCTGCCTACTGCCTCGGA | | GCUGCCUACUGCCUCGGA | |
| | | CTTCAAGGGGCTACTTTA | | CUUCAAGGGGCUACUUUA | |
| | | GGAGCAATTATC | | GGAGCAAUUAUC | |
| shRNA6a | 6 | CCTAGGCAACAGAAGGCT | 2866 | CCUAGGCAACAGAAGGCU | 2878 |
| | | AAAGAAGGTATATTGCTG | | AAAGAAGGUAUAUUGCUG | |
| | | TTGACAGTGAGCGACGTT | | UUGACAGUGAGCGACGUU | |
| | | CATTTGGCAGAAAGAACT | | CAUUUGGCAGAAAGAACU | |
| | | GTGAAGCCACAGATGGGT | | GUGAAGCCACAGAUGGGU | |
| | | TCTTTCTGCCAAATGAAC | | UCUUUCUGCCAAAUGAAC | |
| | | GCTGCCTACTGCCTCGGA | | GCUGCCUACUGCCUCGGA | |
| | | CTTCAAGGGGCTACTTTA | | CUUCAAGGGGCUACUUUA | |
| | | GGAGCAATTATC | | GGAGCAAUUAUC | |
| shRNA7a | 7 | CCTAGGCAACAGAAGGCT | 2867 | CCUAGGCAACAGAAGGCU | 2879 |
| | | AAAGAAGGTATATTGCTG | | AAAGAAGGUAUAUUGCUG | |
| | | TTGACAGTGAGCGACGCA | | UUGACAGUGAGCGACGCA | |
| | | TCAATTACTTCAAAGACT | | UCAAUUACUUCAAAGACU | |
| | | GTGAAGCCACAGATGGGT | | GUGAAGCCACAGAUGGGU | |
| | | CTTTGAAGTAATTGATGC | | CUUUGAAGUAAUUGAUGC | |
| | | GCTGCCTACTGCCTCGGA | | GCUGCCUACUGCCUCGGA | |
| | | CTTCAAGGGGCTACTTTA | | CUUCAAGGGGCUACUUUA | |
| | | GGAGCAATTATC | | GGAGCAAUUAUC | |
| shRNA8a | 8, 89, | CCTAGGCAACAGAAGGCT | 2868 | CCUAGGCAACAGAAGGCU | 2880 |
| | 90-116 | AAAGAAGGTATATTGCTG | | AAAGAAGGUAUAUUGCUG | |
| | | TTGACAGTGAGCGACGTA | | UUGACAGUGAGCGACGUA | |
| | | CCTGATCATCAATTAACT | | CCUGAUCAUCAAUUAACU | |
| | | GTGAAGCCACAGATGGGT | | GUGAAGCCACAGAUGGGU | |
| | | TAATTGATGATCAGGTAC | | UAAUUGAUGAUCAGGUAC | |
| | | GCTGCCTACTGCCTCGGA | | GCUGCCUACUGCCUCGGA | |
| | | CTTCAAGGGGCTACTTTA | | CUUCAAGGGGCUACUUUA | |
| | | GGAGCAATTATC | | GGAGCAAUUAUC | |
| shRNA9a | 9 | CCTAGGCAACAGAAGGCT | 2869 | CCUAGGCAACAGAAGGCU | 2881 |
| | | AAAGAAGGTATATTGCTG | | AAAGAAGGUAUAUUGCUG | |
| | | TTGACAGTGAGCGACGCT | | UUGACAGUGAGCGACGCU | |
| | | GATCTTCGAGAATCTACT | | GAUCUUCGAGAAUCUACU | |
| | | GTGAAGCCACAGATGGGT | | GUGAAGCCACAGAUGGGU | |
| | | AGATTCTCGAAGATCAGC | | AGAUUCUCGAAGAUCAGC | |
| | | GCTGCCTACTGCCTCGGA | | GCUGCCUACUGCCUCGGA | |
| | | CTTCAAGGGGCTACTTTA | | CUUCAAGGGGCUACUUUA | |
| | | GGAGCAATTATC | | GGAGCAAUUAUC | |
| shRNA10a | 10 | CCTAGGCAACAGAAGGCT | 2870 | CCUAGGCAACAGAAGGCU | 2882 |
| | | AAAGAAGGTATATTGCTG | | AAAGAAGGUAUAUUGCUG | |
| | | TTGACAGTGAGCGACGGA | | UUGACAGUGAGCGACGGA | |
| | | AGAAGGGCAAGAAGTACT | | AGAAGGGCAAGAAGUACU | |
| | | GTGAAGCCACAGATGGGT | | GUGAAGCCACAGAUGGGU | |
| | | ACTTCTTGCCCTTCTTCC | | ACUUCUUGCCCUUCUUCC | |
| | | GCTGCCTACTGCCTCGGA | | GCUGCCUACUGCCUCGGA | |
| | | CTTCAAGGGGCTACTTTA | | CUUCAAGGGGCUACUUUA | |
| | | GGAGCAATTATC | | GGAGCAAUUAUC | |
| shRNA11a | 11 | CCTAGGCAACAGAAGGCT | 2871 | CCUAGGCAACAGAAGGCU | 2883 |
| | | AAAGAAGGTATATTGCTG | | AAAGAAGGUAUAUUGCUG | |
| | | TTGACAGTGAGCGACGGA | | UUGACAGUGAGCGACGGA | |
| | | TCAACGAGAAGAAAGACT | | UCAACGAGAAGAAAGACU | |
| | | GTGAAGCCACAGATGGGT | | GUGAAGCCACAGAUGGGU | |
| | | CTTTCTTCTCGTTGATCC | | CUUUCUUCUCGUUGAUCC | |
| | | GCTGCCTACTGCCTCGGA | | GCUGCCUACUGCCUCGGA | |
| | | CTTCAAGGGGCTACTTTA | | CUUCAAGGGGCUACUUUA | |
| | | GGAGCAATTATC | | GGAGCAAUUAUC | |
| shRNA12a | 12 | CCTAGGCAACAGAAGGCT | 2872 | CCUAGGCAACAGAAGGCU | 2884 |
| | | AAAGAAGGTATATTGCTG | | AAAGAAGGUAUAUUGCUG | |
| | | TTGACAGTGAGCGACGGA | | UUGACAGUGAGCGACGGA | |
| | | AGAGTTCCAGAAAGAACT | | AGAGUUCCAGAAAGAACU | |
| | | GTGAAGCCACAGATGGGT | | GUGAAGCCACAGAUGGGU | |
| | | TCTTTCTGGAACTCTTCC | | UCUUUCUGGAACUCUUCC | |
| | | GCTGCCTACTGCCTCGGA | | GCUGCCUACUGCCUCGGA | |
| | | CTTCAAGGGGCTACTTTA | | CUUCAAGGGGCUACUUUA | |
| | | GGAGCAATTATC | | GGAGCAAUUAUC | |

*Sequences of the shRNA constructs as transcribed as part of the eGFP UTR are shown. The expected guide strand resulting from each shRNA is shown in bold.

### Western Blot Analysis:

12-well plates were seeded with 0.25E6 HEK293T cells/well. 24 hours after plating, cells were transfected with 500 ng of shRNA transgene plasmid. Cells were harvested 72 hours post-transfection, pelleted, and lysed. Lysate was clarified by centrifugation, and the protein concentration in each lysate was measured with a Pierce 660 assay. 15 μg of whole cell lysate was loaded into each well of a BioRad TGX Stain-Free gel. When the dye front reached the bottom of the gel, proteins were transferred to a PVDF membrane, blocked and blotted overnight at 4° C. with rabbit anti-HA (1:1000). Following primary antibody incubation, the membrane was rinsed and washed, then blotted with goat anti-rabbit (1:5000) for 1 hour at room temperature. The membrane was developed using 1 mL of Clarity Western ECL Substrate (BioRad), and imaged, the HRP reaction quenched, and re-blotted according to standard methods. The membrane was blotted overnight at 4° C. with Ms.uGAPDH (1:1000). And then later incubated with Gt.uMs (1:5000) for 1 hour at room temperature. Following secondary antibody incubation, the membrane was washed and developed using 1 mL of Clarity Western ECL Substrate (BioRad). CasX band intensity was normalized to GAPDH.

### Western Blot Quantification:

Band intensities were quantified using the Image Lab software from BioRad. The expression of CasX was normalized internally within each sample to account for differences in loading. To do this, the adjusted band volume of CasX was divided by the adjusted band volume of GAPDH. To determine the expression of CasX in Constructs 1-12 as a fraction of Construct 29, the normalized band volume of Constructs 1-12 was divided by the normalized band volume of Construct 29. Lastly, to determine the fold-knockdown of CasX, the normalized expression of CasX in Construct 29 was divided by the normalized expression of CasX in Constructs 1-12.

### Results:

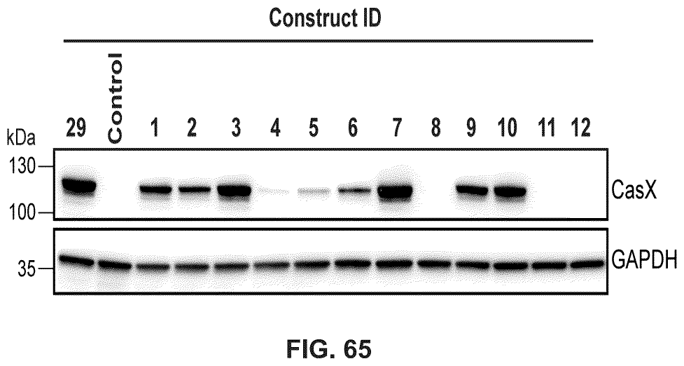

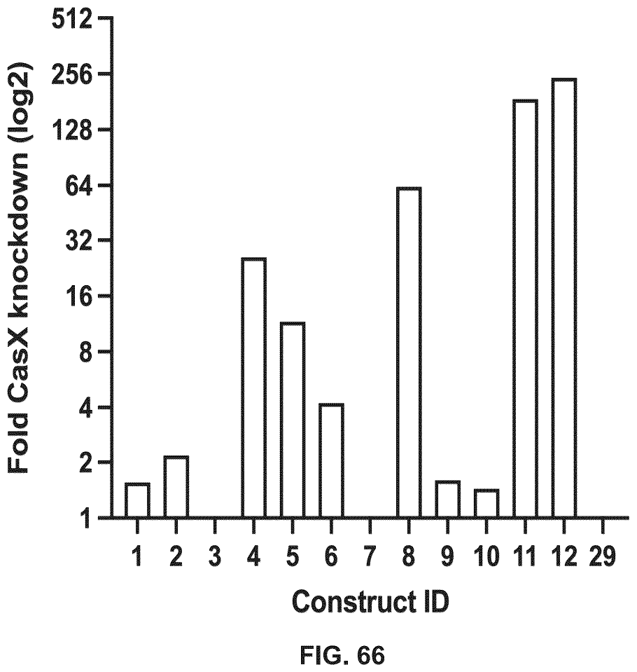

The results show that the incorporation of shRNAs into the AAV system mediated silencing of CasX protein expression to varying degrees. shRNAs 8, 11, 12 (constructs 8, 11, 12) silenced expression down to the level observed in the untransfected protein control (64, 240, and 250-fold knockdown respectively) while shRNA 3 and 7 (constructs 3, 7) did not have an effect on silencing CasX expression in the AAV production cell line (FIG. 65). As shown by Western blot quantification in FIG. 66, shRNAs 8, 11, and 12 were superior in their ability to silence CasX protein expression.

This experiment demonstrated that shRNAs can be used to silence CasX expression in the AAV production cell line such that the resulting AAV particles would have a higher percentage of intact transgenes capable of expressing functional CasX. In subsequent examples, combining shRNAs (more than one shRNA) on the same plasmid as the AAV transgene sequence and incorporating the shRNA, or multiple shRNAs, on another plasmid in production such as pRepCap were explored.

### Example 18: siAAVs can be Efficiently Packaged with the Use of shRNAs to Repress CRISPR Protein Activity in the Producing Cell Line

The self-cleaving AAV strategy demonstrated in Example 12 requires silencing of CasX during AAV production to avoid premature truncation of the AAV genome during production. An shRNA that efficiently silenced transgene-mediated CasX expression was identified as shRNA8 in Example 17.

The experiments in this example were performed to demonstrate that siAAV-mediated cleavage of AAV genome during packaging can be reduced via silencing of CRISPR proteins during AAV production. Furthermore, experiments were conducted to examine whether altering the shRNA promoter and/or the shRNA scaffold can result in stronger silencing of CasX expression.

### Materials and Methods

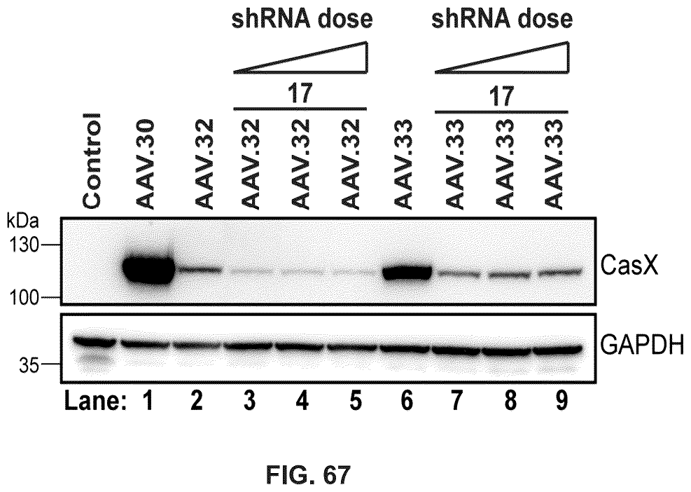

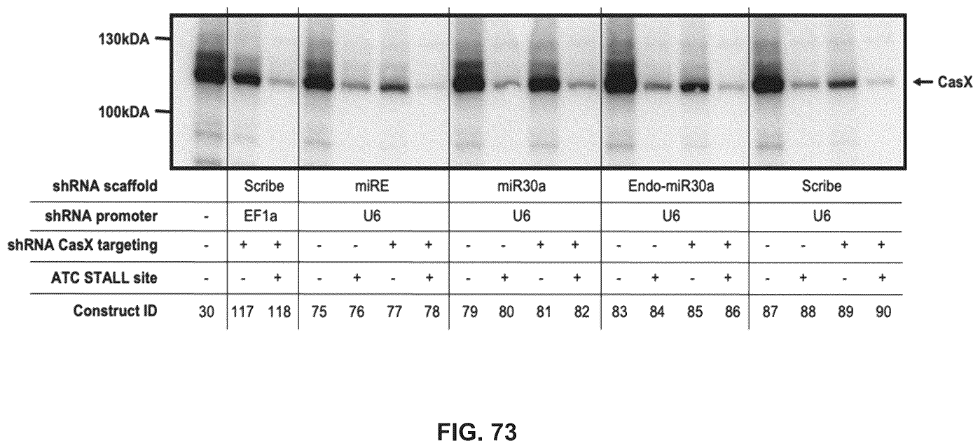

Similar methods, described in Example 17, were used for cloning shRNA sequences to generate constructs testing in this example. For FIGS. 67-71, shRNA8 sequences were cloned into a plasmid containing an EF-1α promoter and GFP reporter cassette (construct ID 17). For FIGS. 73-75, shRNA8 sequences were designed with variants of the miR-30a backbone to direct processing in the cell (versions a-d; Table 28). These shRNA sequences driven by a U6 promoter were cloned into the AatII restriction site on AAV transgene plasmids (construct ID 33). Furthermore, a subset of these resulting plasmids (construct ID 78, 82, 86, 90) were engineered to contain a tdTomato proto-spacer sequence that was preceded by an ATCN PAM (ATCN STALL site) to flank the 5′ end of the CasX nuclease construct, while a subset of plasmids (construct ID 77, 81, 85, 89) did not harbor an ATCN STALL site. As experimental controls, a scrambled shRNA was similarly cloned into plasmids with (construct ID 76, 80, 84, 88) or without the ATCN STALL site (construct ID 75, 79, 83, 87). Table 28 shows sequences of shRNA8 designed with variations in the miR-30a backbone (versions a-d) for intracellular processing. Version ‘a’ uses a miR-Scribe backbone; version ‘b’ uses miR-E; version ‘c’ uses miR-endo; version ‘d’ uses miR-30a.

TABLE 28
Sequences of shRNA8 designed with variations in the miR-30a backbone

| | siAAV | | | | |
|---|---|---|---|---|---|
| | construct | | SEQ ID | | SEQ ID |
| Name | ID | DNA sequence | NO: | RNA sequence* | NO: |
| shRNA8a | 8, 89, | GCCTAGGCAACAGAAGGC | 2885 | GCCUAGGCAACAGAAGGC | 2889 |
| | 90-116 | TAAAGAAGGTATATTGCT | | UAAAGAAGGUAUAUUGCU | |
| | | GTTGACAGTGAGCGACGT | | GUUGACAGUGAGCGACGU | |
| | | ACCTGATCATCAATTAAC | | ACCUGAUCAUCAAUUAAC | |
| | | TGTGAAGCCACAGATGGG | | UGUGAAGCCACAGAUGGG | |
| | | TTAATTGATGATCAGGTA | | UUAAUUGAUGAUCAGGUA | |
| | | CGCTGCCTACTGCCTCGG | | CGCUGCCUACUGCCUCGG | |
| | | ACTTCAAGGGGCTACTTT | | ACUUCAAGGGGCUACUUU | |
| | | AGGAGCAATTATCTTTTT | | AGGAGCAAUUAUCUUUUU | |
| | | TT | | UU | |
| shRNA8b | 77, 78, 91, | GCCTAGGCAACAGAAGGC | 2886 | GCCUAGGCAACAGAAGGC | 2890 |
| | 92, 95, 96, | TCGAGAAGGTATATTGCT | | UCGAGAAGGUAUAUUGCU | |
| | 98, 99, | GTTGACAGTGAGCGTCCT | | GUUGACAGUGAGCGUCCU | |
| | 102-105, | GTACCTGATCATCAATTA | | GUACCUGAUCAUCAAUUA | |
| | 108, 109, | TAGTGAAGCCACAGATGT | | UAGUGAAGCCACAGAUGU | |
| | 111, 112, | ATAATTGATGATCAGGTA | | AUAAUUGAUGAUCAGGUA | |
| | 115, 116 | CAGGTTGCCTACTGCCTC | | CAGGUUGCCUACUGCCUC | |
| | | GGACTTCAAGGGGGCTAC | | GGACUUCAAGGGGGCUAC | |
| | | TTTAGGAGCAATTATCTT | | UUUAGGAGCAAUUAUCUU | |
| | | TTTTT | | UUUUU | |
| shRNA8c | 85, 86, | GCCTAGGCAACAGAATGC | 2887 | GCCUAGGCAACAGAAUGC | 2891 |
| | 93-96, | TAAAGAAGGTATATTGCT | | UAAAGAAGGUAUAUUGCU | |
| | 100-103, | GTTGACAGTGAGCGACTA | | GUUGACAGUGAGCGACUA | |
| | 106-109, | ATTGATGATCAGGTACAG | | AUUGAUGAUCAGGUACAG | |
| | 113-116 | GTCTGTGAAGCCACAGAT | | GUCUGUGAAGCCACAGAU | |
| | | GGGTCCTGTACCATCATC | | GGGUCCUGUACCAUCAUC | |
| | | AATTAGCTGCCTACTGCC | | AAUUAGCUGCCUACUGCC | |
| | | TCGGACTTCAAGGGGCTA | | UCGGACUUCAAGGGGCUA | |
| | | CTTTAGGAGCAATTATCT | | CUUUAGGAGCAAUUAUCU | |
| | | TTTTTT | | UUUUUU | |
| shRNA8d | 81, 82 | GCCTAGGCAACAGAAGGC | 2888 | GCCUAGGCAACAGAAGGC | 2892 |
| | | TCGAGAAGGTATATTGCT | | UCGAGAAGGUAUAUUGCU | |
| | | GTTGACAGTGAGCGTCCT | | GUUGACAGUGAGCGUCCU | |
| | | GTACCTGATCATCAATTA | | GUACCUGAUCAUCAAUUA | |
| | | TAGTGAAGCCACAGATGT | | UAGUGAAGCCACAGAUGU | |
| | | ATAATTGATGATCAGGTA | | AUAAUUGAUGAUCAGGUA | |
| | | CAGGTTGCCTACTGCCTC | | CAGGUUGCCUACUGCCUC | |
| | | GGAATTCAAGGGGGCTAC | | GGAAUUCAAGGGGGCUAC | |
| | | TTTAGGAGCAATTATCTT | | UUUAGGAGCAAUUAUCUU | |
| | | TTTTT | | UUUUU | |

*The expected guide strand resulting from each shRNA is shown in bold.

AAV production: Vectors were produced, and their titer was determined as described in Example 12. Cell crude lysates were obtained as described in Example 17. Sequences of the various components of the vectors are listed in Tables 64-66.

### Western Blot Analysis and Quantification by Densitometry:

The protein concentration in the AAV crude lysates was measured with a Pierce 660 assay. The assay and quantification were performed as described in Example 17. For the western blot analysis in FIG. 73, total protein stain was also performed.

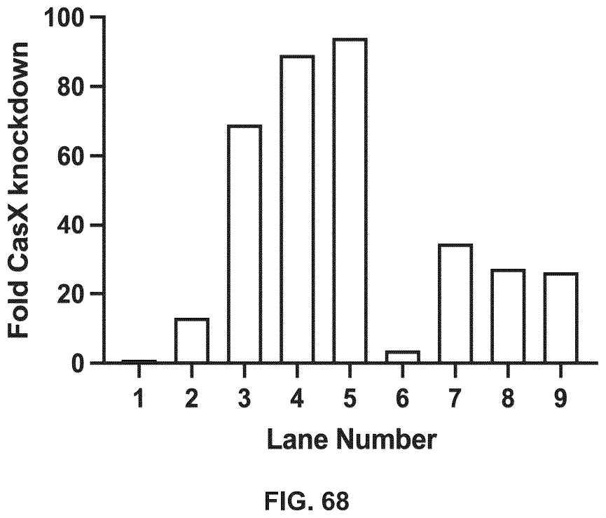

For FIGS. 67 and 68, to determine the expression of CasX in Lanes 2-9 as a fraction of Lane 1, the normalized band volume of Lanes 2-9 was divided by the normalized band volume of Lane 1. Lastly, to determine the fold knockdown of CasX, the normalized expression of CasX in Lane 1 was divided by the normalized expression of CasX in Lanes 2-9.

For FIGS. 74A and 74B, the expression of CasX was normalized to total protein to account for loading differences. To determine the fold knockdown of CasX, the normalized expression of CasX in Lane 1 was divided by the normalized expression of CasX in Lanes 2-19. The experimental condition in Lane 1 is the benchmarking control (AAVs produced from a construct that did not contain an shRNA transcriptional unit or an ATC STALL site).

Assessment of ssAAV Genome by NGS:

To assess the presence of edits in the packaged ssAAV genome, ssDNA was isolated from crude lysate or purified viruses by DNase I digest followed by Proteinase K incubation. 1-5 μL of ssDNA was used for amplification of AAV transgene region flanking the self-inactivating on-target spacer. The rest of the protocol was conducted per Example 12.

### Results:

The results shown in FIG. 67 and FIG. 68 demonstrate that shRNA supplementation during viral packing efficiently silenced transgene-mediated CasX expression in the producer cell line. FIG. 66 shows selected blots for antibodies specific to the CasX protein and GAPDH for normalization. Quantification of CasX knockdown levels in FIG. 68 shows that shRNA expression achieves an almost 100-fold decrease in CasX levels with shRNA8 supplementation (3:1 ratio of shRNA to transgene plasmid) and 30-fold for self-inactivating AAV.32 and AAV.33, respectively.

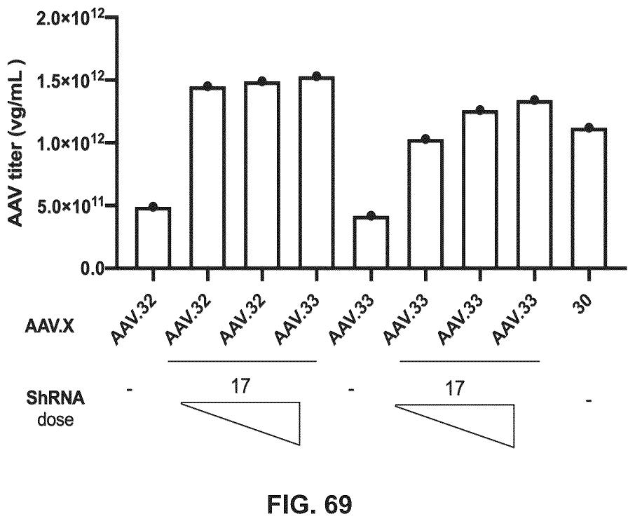

These results were supported by the experimental results shown in FIG. 69, which demonstrated that CasX expression knockdown correlated with increased siAAV titers. siAAVs produced without shRNA supplementation yielded 2-3-fold lower viral genomes encapsulated than siAAVs produced with shRNA supplementation in a dose-dependent manner.

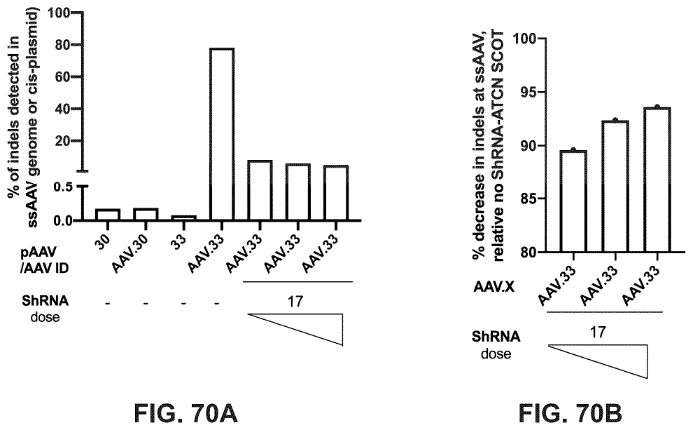

The results shown in FIGS. 70A and 70B confirmed this finding: NGS analysis of the ssDNA packaged in self-inactivating AAV (AAV.33), showing that a 1:1, 1:2 or 1:3 ratio of shRNA:transgene supplemented during production led to a 90% decrease in indel rate detected in packaged ssDNA of siAAV.

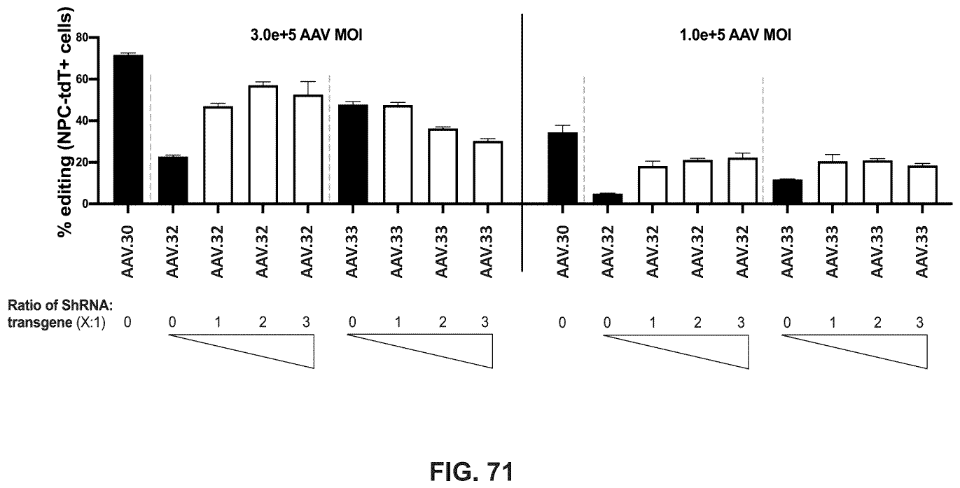

shRNA-mediated decrease in self-inactivation during production improved potency of siAAV vectors as shown in FIG. 71, with over 2-fold improvement in editing relative to the transduced mNPC-tdT cells with siAAV (no shRNA supplementation during production, AAV.32, AAV33). Editing levels were more significantly improved in self-inactivation harboring two self-cleaving fragments flanking the CasX protein (AAV.32) compared to a single self-cleaving fragment at the promoter-protein junction (AAV.33), as expected.

FIG. 73 is a western blot that shows silencing of CasX expression during AAV production using AAVs that were produced from various constructs containing shRNA8. The western blot quantification of normalized CasX expression knockdown for the ‘no STALL’ and ‘ATC STALL’ constructs were illustrated in FIGS. 74A and 74B, respectively. The results in FIGS. 73 and 74 demonstrate that use of the U6 promoter to drive shRNA8 expression resulted in nearly two-fold improvement in CasX knockdown during production compared to that seen with the EF1α promoter (compare construct ID 89 and 90 with construct ID 117 and 118). Furthermore, use of constructs with an ATC STALL site resulted in a marked decrease of CasX protein levels compared to the levels detected when using constructs without a STALL site (‘No STALL’) (FIGS. 73, 74A, 74B). When comparing shRNA scaffolds (i.e., versions a-d, which contain variations in the miR-30a backbone), use of the miR-E (construct ID 75-78) and miR-Scribe (construct ID 87-90) backbones both resulted in stronger CasX knockdown compared to knockdown levels seen when using the miR-30a (construct ID 79-82) or miR-endo backbones (construct ID 83-86). These CasX knockdown findings were seen in both the ‘No STALL’ (FIG. 74A) and ‘ATC STALL’ (FIG. 74B) configuration conditions.

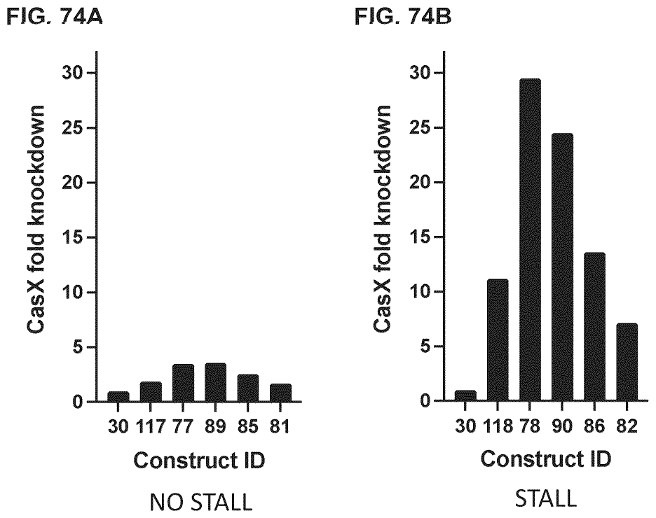

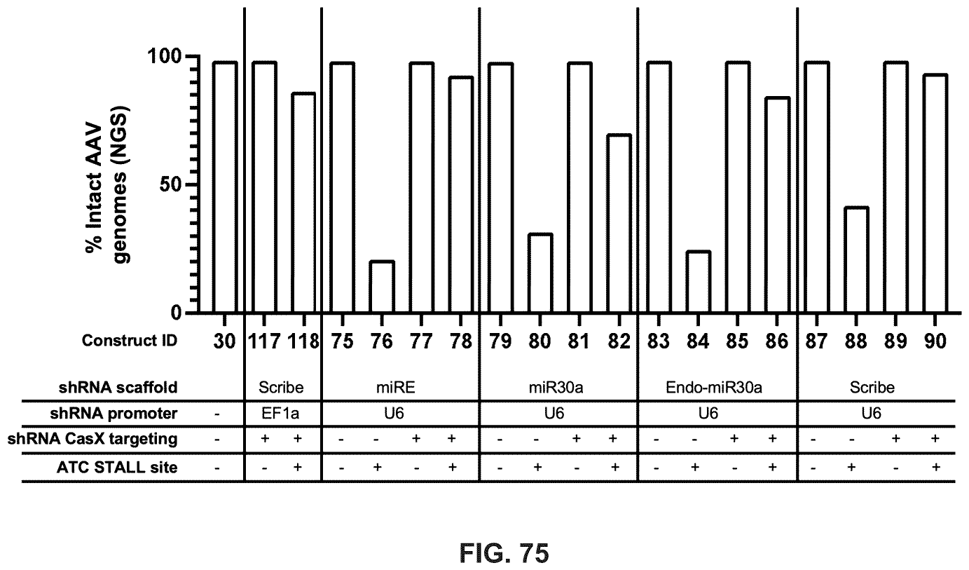

The data presented in FIG. 75 support the findings from the results presented in FIGS. 73 and 74; i.e., use of shRNA8 and a STALL site resulted in more intact AAV genomes during AAV production compared to conditions that incorporated just a STALL site. NGS analysis of the ssDNA packaged in siAAV supplemented with shRNA8 using a miR-E (construct ID 78) and miR-Scribe (construct ID 90) backbone and driven by U6 promoter resulted in more intact AAV genomes compared to that seen in the miR-30a (construct ID 82) and miR-endo (construct ID 86) conditions. Furthermore, using the U6 promoter with the miR-Scribe backbone (construct ID 90) resulted in ˜96% of AAV genomes that were intact, an improvement over the 88.3% seen with the corresponding EF1α promoter condition (construct ID 78). As anticipated, with the NO STALL experimental control constructs, at least ˜99% of the genome remained intact (FIG. 75).

These experiments demonstrate that shRNA8 expression during AAV production promotes a rescue of full capsid ratio during packaging, with an increase in titer correlating with increased expression of the shRNA in the producer cell line during AAV packaging. NGS and potency assay confirmed that shRNA supplementation significantly decreased self-inactivation and, therefore, more intact AAV genomes during AAV production. Furthermore, improvements in reducing CasX-mediated self-cleavage resulting in more intact AAV genomes during production can be seen when using a better-performing promoter and/or miR scaffold for shRNA expression. In addition, the results of this experiment demonstrate that inclusion of a STALL site can effectively result in CasX self-inactivation by markedly decreasing CasX protein levels, which would minimize the potential of inducing off-target effects and unwanted immunogenic outcomes.

### Example 19: Assessing Differential PAM Recognition In Vitro

### 1. Comparison of Reference and CasX Variants

In vitro cleavage assays were performed with RNPs using CasX2, CasX119, and CasX438 complexed with sg174.7.37. Fluorescently labeled dsDNA targets with a 7.37 spacer and either a TTC, CTC, GTC, or ATC PAM were used (sequences shown in Table 29). Cleavage reactions were prepared with final RNP concentrations of 100 nM and a final target concentration of 100 nM. Reactions were carried out at 370C and initiated by the addition of the 7.37 target DNA. Time points were taken at 0.25, 0.5, 1, 2, 5, 10, 30, and 60 minutes and were quenched by adding to 95% formamide, 20 mM EDTA. Samples were heat-denatured, run on a 10% urea PAGE gel, and imaged and quantified according to standard methods. Apparent first-order rate constants for non-target strand cleavage (k_{cleave}) were determined for each CasX:sgRNA complex on each target. Rate constants for targets with non-TTC PAM were compared to the TTC PAM target to determine whether the relative preference for each PAM was altered in a given protein variant.

For all variants, the TTC target supported the highest cleavage rate, followed by the ATC, then the CTC, and finally the GTC target (FIGS. 76A and B, Table 30). For each combination of CasX variant and NTC PAM, the cleavage rate kcleave is shown. For all non-NTC PAMs, the relative cleavage rate as compared to the TTC rate for that variant is shown in parentheses. All non-TTC PAMs exhibited substantially decreased cleavage rates (>10-fold for all). The ratio between the cleavage rate of a given non-TTC PAM and the TTC PAM for a specific variant remained generally consistent across all variants. The CTC target supported cleavage 3.5-4.3% as fast as the TTC target; the GTC target supported cleavage 1.0-1.4% as fast; and the ATC target supported cleavage 6.5-8.3% as fast. The exception was 491, where the kinetics of cleavage at TTC PAMs were too fast to allow accurate measurement, which artificially decreased the apparent difference between TTC and non-TTC PAMs. Comparing the relative rate of 491 with GTC, CTC, and ATC PAMs resulted in ratios comparable to those for other variants when comparing across non-TTC PAMs, consistent with the rates increasing in tandem. Overall, differences between the variants were not substantial enough to suggest that the relative preference for the various NTC PAMs were altered. However, the higher basal cleavage rates of the variants allowed targets with ATC or CTC PAMs to be cleaved nearly completely within 10 minutes, and the apparent k_{cleaves} were comparable to or greater than the k_{cleave} of CasX2 on a TTC PAM (Table 30). This increased cleavage rate may cross the threshold necessary for effective genome editing in a human cell, explaining the apparent increase in PAM flexibility for these variants.

TABLE 29
Sequences of DNA substrates used in in vitro PAM cleavage assay.

| Guide* | DNA Sequence | SEQ ID NO |
|---|---|---|
| 7.37 TTC PAM TS | AGCGCGAGCACAGCTAAGGCCACGGAGCGAGACATCTCGG | 723 |
| | CCCGAATGCTGTCAGCTTCA | |
| 7.37 TTC PAM NTS | TGAAGCTGACAGCATTCGGGCCGAGATGTCTCGCTCCGTG | 724 |
| | GCCTTAGCTGTGCTCGCGCT | |
| 7.37 CTC PAM TS | AGCGCGAGCACAGCTAAGGCCACGGAGCGAGACATCTCGG | 725 |
| | CCCGAGTGCTGTCAGCTTCA | |
| 7.37 CTC PAM NTS | TGAAGCTGACAGCACTCGGGCCGAGATGTCTCGCTCCGTG | 726 |
| | GCCTTAGCTGTGCTCGCGCT | |
| 7.37 GTC PAM TS | AGCGCGAGCACAGCTAAGGCCACGGAGCGAGACATCTCGG | 727 |
| | CCCGACTGCTGTCAGCTTCA | |
| 7.37 GTC PAM NTS | TGAAGCTGACAGCAGTCGGGCCGAGATGTCTCGCTCCGTG | 728 |
| | GCCTTAGCTGTGCTCGCGCT | |
| 7.37 ATC PAM TS | AGCGCGAGCACAGCTAAGGCCACGGAGCGAGACATCTCGG | 729 |
| | CCCGATTGCTGTCAGCTTCA | |
| 7.37 ATC PAM NTS | TGAAGCTGACAGCAATCGGGCCGAGATGTCTCGCTCCGTG | 730 |
| | GCCTTAGCTGTGCTCGCGCT | |

*The PAM sequences for each are bolded. TS-target strand. NTS-Non-target strand.

TABLE 30
Apparent cleavage rates of CasX variants against NTC PAMs

| Variant | TTC | | CTC | GTC | ATC |
|---|---|---|---|---|---|
| 2 | 0.267 | min⁻¹ | 9.29E−3 min⁻¹ | 3.75E−3 min⁻¹ | 1.87E−2 min⁻¹ |
| | | | (0.035) | (0.014) | (0.070) |
| 119 | 8.33 | min⁻¹ | 0.303 min⁻¹ | 8.64E−2 min⁻¹ | 0.540 min⁻¹ |
| | | | (0.036) | (0.010) | (0.065) |
| 438 | 4.94 | min⁻¹ | 0.212 min⁻¹ | 1.31E−2 min⁻¹ | 0.408 min⁻¹ |
| | | | (0.043) | (0.013) | (0.083) |
| 491 | 16.42 | min⁻¹ | 8.605 min⁻¹ | 2.447 min⁻¹ | 11.33 min⁻¹ |
| | | | (0.524) | (0.149) | (0.690) |

### 2. Comparison of PAM Recognition Using Single CasX Variant

Materials and Methods: Fluorescently labeled dsDNA targets with a 7.37 spacer and either a TTC, CTC, GTC, ATC, TTT, CTT, GTT, or ATT PAM were used (sequences are in Table 31). Oligos were ordered with a 5′ amino modification and labeled with a Cy7.5 NHS ester for target strand oligos and a Cy5.5 NHS ester for non-target strand oligos. dsDNA targets were formed by mixing the oligos in a 1:1 ratio in 1× cleavage buffer (20 mM Tris HCl pH 7.5, 150 mM NaCl, 1 mM TCEP, 5% glycerol, 10 mM MgCl2), heating to 95° C. for 10 minutes, and allowing the solution to cool to room temperature.

CasX variant 491 was complexed with sg174.7.37. The guide was diluted in 1× cleavage buffer to a final concentration of 1.5 μM, and then protein was added to a final concentration of 1 μM. The RNP was incubated at 37° C. for 10 minutes and then put on ice.

Cleavage assays were carried out by diluting RNP in cleavage buffer to a final concentration of 200 nM and adding dsDNA target to a final concentration of 10 nM. Time points were taken at 0.25, 0.5, 1, 2, 5, and 10 minutes and quenched by adding to an equal volume of 95% formamide and 20 mM EDTA Results

The relative cleavage rate of the 491.174 RNP on various PAMs was investigated. In addition to aiding in the prediction of cleavage efficiencies of targets and potential off-targets in cells, these data allowed for the adjustment of the cleavage rate for synthetic targets. In the case of self-limiting AAV vectors, where new protospacers can be added within the vector to allow for self-targeting, it was reasoned that the rate of episome cleavage could be adjusted up or down by changing the PAM.

The cleavage rate of the RNP was tested against various dsDNA substrates that were identical in sequence aside from the PAM. This experimental setup allowed for the isolation of the effects of the PAM itself, rather than convoluting PAM recognition with effects resulting from spacer sequence and genomic context. All NTC and NTT PAMs were tested. As expected, the RNP cleaved the target with the TTC PAM most quickly, converting essentially all of it to product by the first time point (FIG. 76A). CTC was cleaved roughly half as quickly, though the rapid cleavage of TTC makes determining an accurate k_{cleave} difficult under these assay conditions, which are optimized to capture a broader array of cleavage rates (Table 32). The GTC target was cleaved most slowly of the NTC PAMs, with a cleavage rate roughly six-fold slower than the TTC target. All NTT PAMs were cleaved more slowly than all NTC PAMs (FIG. 76B), with TTT cut most efficiently, followed by GTT (Table 32). The relative efficiency of GTT cleavage among all NTT PAMs, compared to the low rate of GTC cleavage compared to all NTC PAMs, demonstrating that recognition of individual PAM nucleotides was context-dependent, with nucleotide identity at one position in the PAM affecting sequence preference at the other positions.

The PAM sequences tested here yielded cleavage rates spanning three orders of magnitude while still maintaining cleavage activity at the same spacer sequence. These data demonstrated that cleavage rates at a given synthetic target can be readily modified by changing the associated PAM, allowing for adjustment of self-cleavage activity and efficient targeting of the genomic target, prior to cleavage and elimination of the AAV episome.

TABLE 31
Sequences of DNA substrates used in in vitro PAM cleavage assay*

| | SEQ ID | |
|---|---|---|
| PAM & Strand | NO | Spacer and PAM Sequence |
| 7.37 TTC PAM TS | 731 | AGCGCGAGCACAGCTAAGGCCACGGAGCGAGACATCTCGGCCCGAATGCTG |
| | | TCAGCTTCA |
| 7.37 TTC PAM NTS | 732 | TGAAGCTGACAGCATTCGGGCCGAGATGTCTCGCTCCGTGGCCTTAGCTGT |
| | | GCTCGCGCT |
| 7.37 CTC PAM TS | 733 | AGCGCGAGCACAGCTAAGGCCACGGAGCGAGACATCTCGGCCCGAGTGCTG |
| | | TCAGCTTCA |
| 7.37 CTC PAM NTS | 734 | TGAAGCTGACAGCACTCGGGCCGAGATGTCTCGCTCCGTGGCCTTAGCTGT |
| | | GCTCGCGCT |
| 7.37 GTC PAM TS | 735 | AGCGCGAGCACAGCTAAGGCCACGGAGCGAGACATCTCGGCCCGACTGCTG |
| | | TCAGCTTCA |
| 7.37 GTC PAM NTS | 736 | TGAAGCTGACAGCAGTCGGGCCGAGATGTCTCGCTCCGTGGCCTTAGCTGT |
| | | GCTCGCGCT |
| 7.37 ATC PAM TS | 737 | AGCGCGAGCACAGCTAAGGCCACGGAGCGAGACATCTCGGCCCGATTGCTG |
| | | TCAGCTTCA |
| 7.37 ATC PAM NTS | 738 | TGAAGCTGACAGCAATCGGGCCGAGATGTCTCGCTCCGTGGCCTTAGCTGT |
| | | GCTCGCGCT |
| 7.37 TTT PAM TS | 739 | AGCGCGAGCACAGCTAAGGCCACGGAGCGAGACATCTCGGCCCAAATGCTG |
| | | TCAGCTTCA |
| 7.37 TTT PAM NTS | 740 | TGAAGCTGACAGCATTTGGGCCGAGATGTCTCGCTCCGTGGCCTTAGCTGT |
| | | GCTCGCGCT |
| 7.37 CTT PAM TS | 741 | AGCGCGAGCACAGCTAAGGCCACGGAGCGAGACATCTCGGCCCTAGTGCTG |
| | | TCAGCTTCA |
| 7.37 CTT PAM NTS | 742 | TGAAGCTGACAGCACTTGGGCCGAGATGTCTCGCTCCGTGGCCTTAGCTGT |
| | | GCTCGCGCT |
| 7.37 GTT PAM TS | 743 | AGCGCGAGCACAGCTAAGGCCACGGAGCGAGACATCTCGGCCCTACTGCTG |
| | | TCAGCTTCA |
| 7.37 GTT PAM NTS | 744 | TGAAGCTGACAGCAGTTGGGCCGAGATGTCTCGCTCCGTGGCCTTAGCTGT |
| | | GCTCGCGCT |
| 7.37 ATT PAM TS | 745 | AGCGCGAGCACAGCTAAGGCCACGGAGCGAGACATCTCGGCCCTATTGCTG |
| | | TCAGCTTCA |
| 7.37 ATT PAM NTS | 746 | TGAAGCTGACAGCAATTGGGCCGAGATGTCTCGCTCCGTGGCCTTAGCTGT |
| | | GCTCGCGCT |

*The DNA sequences used to generate each dsDNA substrate are shown. The PAM sequences for each are bolded. TS-target strand. NTS-Non-target strand.

TABLE 32
Apparent cleavage rates of CasX 491.174 against NTC and NTT PAMs

| PAM | TTC | ATC | CTC | GTC | TTT | ATT | CTT | GTT |
|---|---|---|---|---|---|---|---|---|
| k_{cleave} (min⁻¹) | 15.6* | 6.66 | 9.45 | 2.52 | 1.33 | 0.0675 | 0.0204 | 0.330 |

*The rate of TTC cleavage exceeds the resolution of this assay, so the resulting k_{cleave} should be taken as a lower bound.

### Example 20: The PASS Assay Identifies CasX Protein Variants of Differing PAM Sequence Specificity

The purpose of the experiment was to identify the PAM sequence specificities of CasX proteins 2 (SEQ ID NO: 2), 491 (SEQ ID NO: 138), 515 (SEQ ID NO: 145), 533 (SEQ ID NO: 162), 535 (SEQ ID NO: 164), 668 (SEQ ID NO: 296), and 672 (SEQ ID NO: 299). To accomplish this, the HEK293 cell line PASS_V1.01 or PASS_V1.02 was treated with the above CasX proteins in at least two replicate experiments, and Next-generation sequencing (NGS) was performed to calculate the percent editing using a variety of spacers at their intended target sites.

### Materials and Methods

A multiplexed pooled approach was taken to assay clonal protein variants using the PASS system. Briefly, two pooled HEK cell lines were generated and termed PASS_V1.01 and PASS_V1.02. Each cell within the pool contained a genome-integrated single-guide RNA (sgRNA), paired with a specific target site. After transfection of protein-expression constructs, editing at a specific target by a specific spacer could be quantified by NGS. Each guide-target pair was designed to provide data related to activity, specificity, and targetability of the CasX-guide RNP complex.

Paired spacer-target sequences were synthesized by Twist Biosciences and obtained as an equimolar pool of oligonucleotides. This pool was amplified by PCR and cloned by Golden Gate cloning to generate a final library of plasmids named p77. Each plasmid contained a sgRNA expression element and a target site, along with a GFP expression element. The sgRNA expression element consisted of a U6 promoter driving transcription of gRNA scaffold 174 (SEQ ID NO:2238), followed by a spacer sequence which would target the RNP of the guide and CasX variant to the intended target site. 250 possible unique, paired spacer-target synthetic sequences were designed and synthesized. A pool of lentivirus was then produced from this plasmid library using the LentiX production system (Takara Bio USA, Inc) according to the manufacturer's instructions. The resulting viral preparation was then quantified by qPCR and transduced into a standard HEK293 cell line at a low multiplicity of infection so as to generate single copy integrations. The resulting cell line was then purified by fluorescence-activated cell sorting (FACS) to complete the production of PASS_V1.01 or PASS_V1.02. A cell line was then seeded in six-well plate format and treated in duplicate with either water or was transfected with 2 μg of plasmid p67. Plasmid p67 contains an EF-1alpha promoter driving expression of a CasX protein tagged with the SV40 Nuclear Localization Sequence. After two days, treated cells were collected, lysed, and genomic DNA was extracted using a genomic DNA isolation kit. Genomic DNA was then PCR amplified with custom primers to generate amplicons compatible with Illumina™ NGS and sequenced on a NextSeq instrument. Sample reads were demultiplexed and filtered for quality. Editing outcome metrics (fraction of reads with indels) were then quantified for each spacer-target synthetic sequence across treated samples.

To assess the PAM sequence specificity for a CasX protein, editing outcome metrics for four different PAM sequences were categorized. For TTC PAM target sites, 48 different spacer-target pairs were quantified; for ATC, CTC, and GTC PAM target sites, 14, 22, and 11 individual target sites were quantified, respectively. For some CasX proteins, replicate experiments were repeated dozens of times over several months. For each of these experiments, the average editing efficiency was calculated for each of the above described spacers. The average editing efficiency across the four categories of PAM sequence was then calculated from all such experiments, along with the standard deviation of these measurements.

### Results:

Table 33 lists the average editing efficiency across PAM categories and across CasX protein variants, along with the standard deviation of these measurements. The number of measurements for each category is also indicated. These data indicate that the engineered CasX variants 491 and 515 are specific for the canonical PAM sequence TTC, while other engineered variants of CasX performed more or less efficiently at the PAM sequences tested. In particular, the average rank order of PAM preferences for CasX 491 is TTC>>ATC>CTC>GTC, or TTC a ATC>GTC>CTC for CasX 515, while the wild-type CasX 2 exhibits an average rank order of TTC>>GTC>CTC>ATC. Note that for the lower editing PAM sequences the error of these average measurements is high. In contrast, CasX variants 535, 668, and 672 have considerably broader PAM recognition, with a rank order of TTC>CTC>ATC>GTC. Finally, CasX 533 exhibits a completely re-ordered ranking relative to the WT CasX, ATC>CTC>>GTC>TTC. These data can be used to engineer maximally-active therapeutic CasX molecules for a target DNA sequence of interest.

Under the conditions of the experiments, a set of CasX proteins was identified that are improved for double-stranded DNA cleavage in human cells at target DNA sequences associated with a PAM of sequence TTC, ATC, CTC, or GTC, supporting that CasX variants with an altered spectrum of PAM specificity, relative to wild-type CasX, can be generated.

TABLE 33
Average editing of selected CasX Proteins at spacers associated
with PAM sequences of TTC, ATC, CTC, or GTC

| CasX | PAM | Average Percent | Standard | Number of |
|---|---|---|---|---|
| Name | Sequence | Editing | Deviation | Measurements |
| 2 | ATC | 0.40 | 1.35 | 336 |
| 2 | CTC | 0.46 | 2.29 | 528 |
| 2 | GTC | 0.69 | 6.27 | 264 |
| 2 | TTC | 5.28 | 7.34 | 1152 |
| 491 | ATC | 6.86 | 8.29 | 364 |
| 491 | CTC | 4.54 | 6.40 | 572 |
| 491 | GTC | 3.40 | 6.68 | 286 |
| 491 | TTC | 40.41 | 23.13 | 1248 |
| 515 | ATC | 4.47 | 5.49 | 252 |
| 515 | CTC | 3.36 | 4.80 | 396 |
| 515 | GTC | 3.65 | 10.75 | 198 |
| 515 | TTC | 36.75 | 24.89 | 864 |
| 533 | ATC | 47.50 | 15.86 | 96 |
| 533 | CTC | 25.90 | 14.74 | 28 |
| 533 | GTC | 6.34 | 8.36 | 44 |
| 533 | TTC | 0.87 | 3.05 | 22 |
| 535 | ATC | 9.70 | 10.20 | 56 |
| 535 | CTC | 11.77 | 13.59 | 88 |
| 535 | GTC | 7.62 | 15.04 | 44 |
| 535 | TTC | 29.29 | 18.78 | 192 |
| 668 | ATC | 44.69 | 24.40 | 56 |
| 668 | CTC | 46.14 | 26.57 | 88 |
| 668 | GTC | 30.48 | 24.06 | 44 |
| 668 | TTC | 55.34 | 28.59 | 192 |
| 672 | ATC | 25.51 | 20.85 | 56 |
| 672 | CTC | 30.05 | 22.95 | 88 |
| 672 | GTC | 14.21 | 13.38 | 44 |
| 672 | TTC | 52.36 | 27.64 | 192 |

### Example 21: In Vivo Administration of the siAAV-CasX (siAAV) System Results in Efficient Editing at the Target Locus while Inducing CasX Self-Inactivation In Vivo

Experiments were performed to demonstrate that small CRISPR proteins such as CasX with targeting gRNA can edit the target genomic locus when expressed in vivo from an AAV episome with a self-inactivating system. Furthermore, these experiments showed that the AAV self-inactivation system is capable of reducing CasX expression in vivo to minimize potential off-target effects, thereby enhancing the therapeutic index of the constructs.

### Materials and Methods

AAVs encoding CasX variant 491 with guide scaffold 174 and spacer 12.7 targeting the tdTomato locus in Ai9 transgenic mice were used in these in vivo experiments. AAV construct cloning and AAV production using adherent HEK293T cells were performed as described in Example 1. Sequences of AAV constructs used in this experiment are listed in Table 66.

In Vivo Administration of siAAVs, Tissue Processing, and Immunohistochemistry (IHC):

The Ai9 mouse model was used in this example. Ai9 is a Cre reporter tool strain designed to have a loxP flanked STOP cassette preventing the transcription of a CAG promoter-driven tdTomato marker. These mice express tdTomato following Cre-mediated recombination to remove the STOP cassette. To assess in vivo siAAV editing, neonates from Ai9 reporter mice were injected with siAAV particles encoding CasX variant 491 driven by the UbC promoter and gRNA scaffold variant 174 with spacer 12.7 targeting the tdTomato STOP cassette (refer to Table 66 for sequences). Here, the CasX construct was engineered such that the CasX coding sequence was flanked on both the 5′ and 3′ ends by a tdTomato proto-spacer sequence, which was preceded by either an ATCN or CTCN PAM (labeled as ‘Dual ATC STALL’ or ‘Dual CTC STALL’ respectively). In addition, another CasX construct was designed where the CasX sequence was flanked only on the 5′ end by a tdTomato proto-spacer site with an ATC PAM (‘single ATC STALL’). As a control, a CasX construct without the flanking tdTomato protospacer sequences for self-inactivation was used (‘No STALL’). ‘Dual ATC STALL’ construct is denoted with construct ID 136; ‘Dual CTC STALL’ is denoted with construct ID 137; ‘single ATC STALL’ is denoted with construct ID 90; and ‘no STALL’ is denoted with construct ID 30.

Briefly, ˜4E9 siAAV particles were administered intracerebroventricularly into Ai9 neonates. As another control group, neonates were injected with AAVs (construct ID 147; Table 66) containing CasX 491 and gRNA scaffold 235 with a non-targeting spacer (spacer 0.0, CGAGACGUAAUUACGUCUCG (SEQ ID NO: 2893)). Three weeks and eight weeks post-injection, mice were euthanized by terminal anesthesia followed by transcardiac perfusion. Brains were harvested and cut along the midline into left and right hemispheres. The left hemisphere of the brain was post-fixed in 4% paraformaldehyde at 4° C., followed by infiltration with 30% sucrose solution. Tissues were embedded in the OCT compound and frozen; OCT-embedded brains were cross-sectioned using a cryostat. Sections were counterstained with DAPI to label nuclei, mounted on slides, and imaged on a fluorescent microscope. Editing levels were quantified by counting the number of tdTomato+ cells as a percentage of DAPI-labeled nuclei. The right hemisphere of the brain was harvested and processed for extraction of 1) RNA using the Zymo Quick-RNA™ Miniprep Kit following the manufacturer's instructions and 2) protein using standard molecular techniques involving acetone precipitation.

### Assessment of RNA Levels by RT-qPCR:

RNA extracted from brain tissue was used as input for reverse transcription. The resulting cDNA served as input for qPCR reactions to quantify the amount of transcribed CasX, guide scaffold 174, and tdTomato, using HEX/FAM-based detection with primers-probe sets targeting CasX, guide scaffold 174, or tdTomato. Expression of the ACTB housekeeping gene was used for normalization. Expression data were analyzed according to the double delta Ct method.

### Western Blotting and Protein Quantification:

Extracted tissue protein samples were resolved by SDS-PAGE followed by Western blotting using an in-house polyclonal antibody recognizing CasX to analyze the levels of CasX protein. Western blotting and quantification of band intensity was performed using standard procedures. CasX protein knockdown was determined for each siAAV condition (Dual ATC STALL, Dual CTC STALL, or Single ATC STALL) and normalized to the CasX protein level for the AAV without self-inactivation (No STALL) control.

### Results:

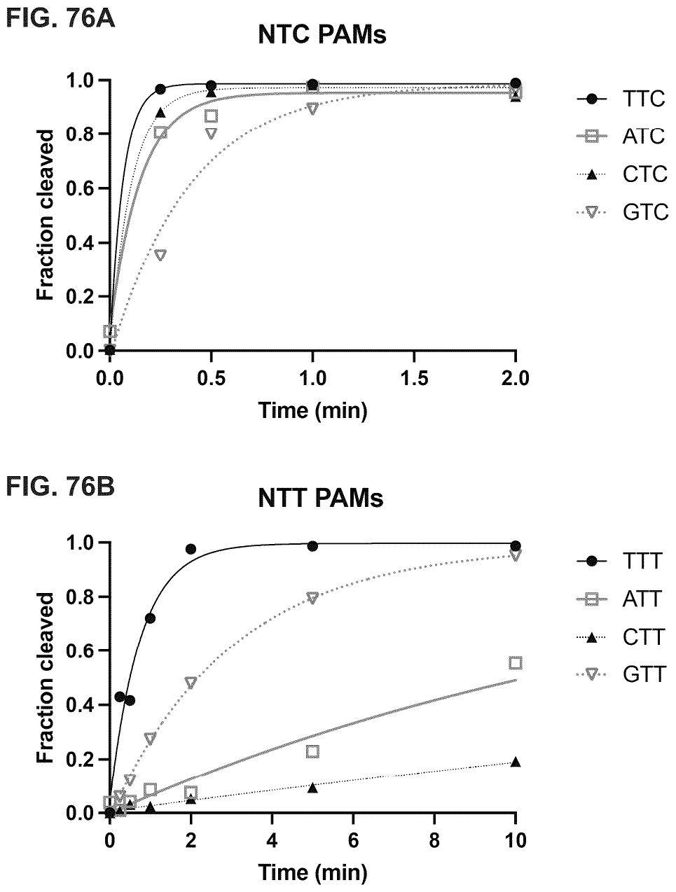

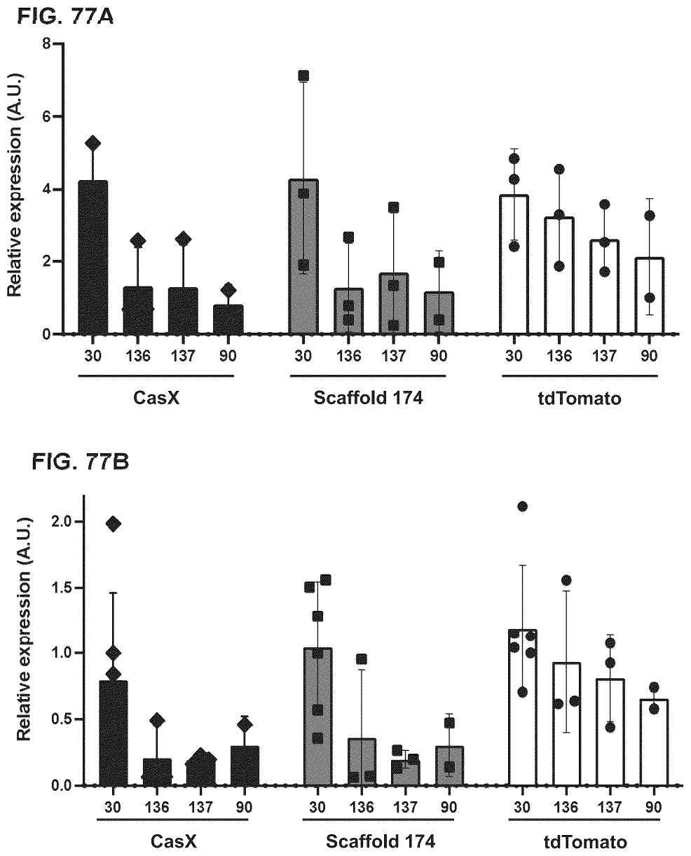

The bar plots in FIGS. 77A and 77B illustrate the quantification of RNA transcript levels for CasX, gRNA scaffold 174, and tdTomato in mouse brain tissue harvested three weeks (FIG. 77A) and eight weeks (FIG. 77B) post-treatment with XAAVs with or without the self-inactivation system. The data illustrate that CasX and gRNA expression in the No STALL group was approximately four times greater than the CasX and gRNA expression exhibited by the Dual ATC STALL group, Dual CTC STALL group, or Single ATC STALL group at both the three-week and eight-week time points (FIGS. 77A and 77B). However, tdTomato expression was comparable across all four groups, regardless of STALL presence. Collectively, these data indicate that siAAV constructs were able to reduce CasX and gRNA expression without significantly affecting editing at the tdTomato locus to induce tdTomato expression, supporting that the design of the constructs permits the desired target editing and subsequent inactivation to reduce the expression of the CasX and gRNA in vivo.

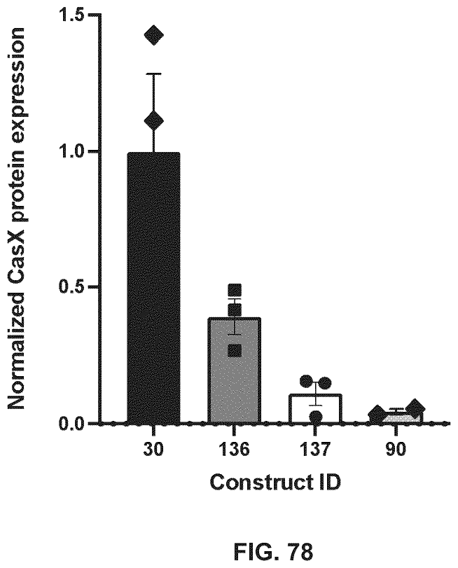

The bar plot in FIG. 78 shows the quantification of CasX protein expression in mouse brain tissue harvested at the three-week time point. These data corroborate the RT-qPCR findings in FIGS. 77A and 77B, such that incorporation of the self-inactivation system (Dual ATC STALL, Dual CTC STALL, or Single ATC STALL) resulted in the reduction of CasX protein levels compared to CasX levels detected in the No STALL group. Specifically, the Dual ATC STALL group exhibited a ˜2.55-fold decrease in CasX protein levels, while the Dual CTC STALL group showed a ˜9.1-fold reduction relative to the CasX levels with the No STALL group (FIG. 78). These results suggest that CasX has a relatively lower binding affinity with ATC PAM compared to CTC PAM, and thus, a weaker self-inactivating activity and lower relative CasX knockdown. Interestingly, the data also suggest that incorporating a single ATC STALL site resulted in more effective reduction of CasX expression than using two STALL sites at the three-week time point (FIG. 78).

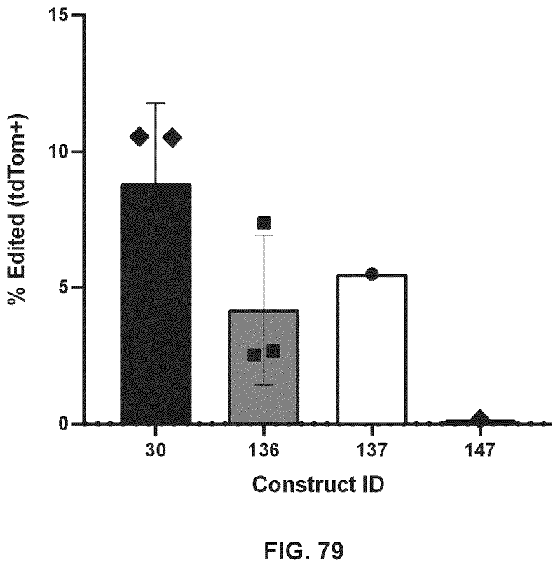

The bar graph in FIG. 79 is a quantification of tdTomato+ cells detected in the harvested brain tissue from mice treated with XAAVs with or without the self-inactivation system. The data show that the percentage of cells expressing tdTomato was similar across all the groups treated with XAAVs containing the tdTomato-targeting spacer, regardless of the presence of self-inactivation (FIG. 79), supporting the RT-qPCR data discussed earlier in FIGS. 77A and 77B. As expected, mice treated with XAAVs containing the non-targeting spacer did not exhibit tdTomato expression (FIG. 79).

These experiments demonstrated that administering siAAVs in vivo can result in efficient editing at the target genomic locus mediated by the CasX:gRNA system, while also effectively modulating CasX expression via the self-inactivation system to minimize potential off-target effects and undesired immunogenicity, enhancing the therapeutic index of the constructs.

### Example 22: Assessment of Using Decoy gRNAs to Rescue siAAV Titers During Production

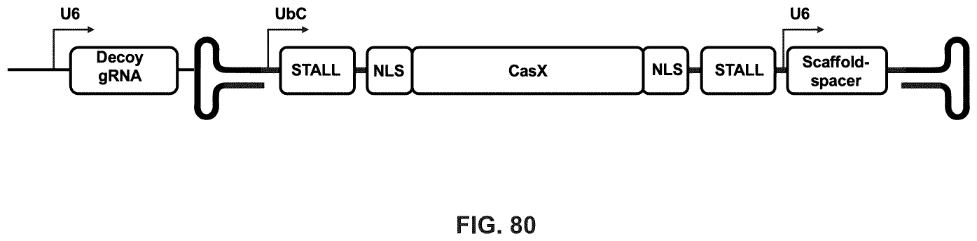

Experiments were performed to investigate alternative strategies that may be employed to circumvent CasX-mediated cleavage of the AAV transgene by reducing CasX editing without decreasing its expression during AAV production. One strategy involves designing decoy gRNAs with a non-targeting spacer or without a spacer, which, upon expression, would complex with the CasX to prevent CasX from targeting the STALL sites flanking the nuclease construct. FIG. 80 illustrates a general configuration of a construct that would encode for a decoy gRNA, supplied on the same plasmid as the AAV transgene, to rescue siAAV titers that remain functional during production.

### Materials and Methods

AAV plasmid cloning was performed using standard molecular cloning methods described in Example 17. Briefly, as illustrated in FIG. 80, the siAAV transgene plasmid was designed to contain a U6 transcriptional unit upstream (5′) of the 5′ ITR, where the construct encoding a decoy gRNA is driven by the U6 promoter. Sequences of the decoy gRNAs designed and investigated in this example are listed in Table 34.

TABLE 34
Sequences of decoy gRNAs with the
indicated guide scaffold variants.

| siAAV | Decoy gRNA design | | |
|---|---|---|---|
| construct ID | (scaffold-spacer) | DNA SEQ ID NO | RNA SEQ ID NO |
| 126 | Scrambled-NT | 2894 | 4007 |
| 127 | Scrambled-NS | 2895 | 4008 |
| 128 | Scrambled-NT | 2896 | 4007 |
| 129 | Scrambled-NS | 2897 | 4008 |
| 130 | 174-NT | 2898 | 4009 |
| 131 | 174-NS | 2899 | 4010 |
| 132 | 234-NT | 2900 | 4011 |
| 133 | 234-NS | 2901 | 4012 |
| 134 | 235-NT | 2902 | 4013 |
| 135 | 235-NS | 2903 | 4014 |

NT = non-targeting spacer;
NS = no spacer

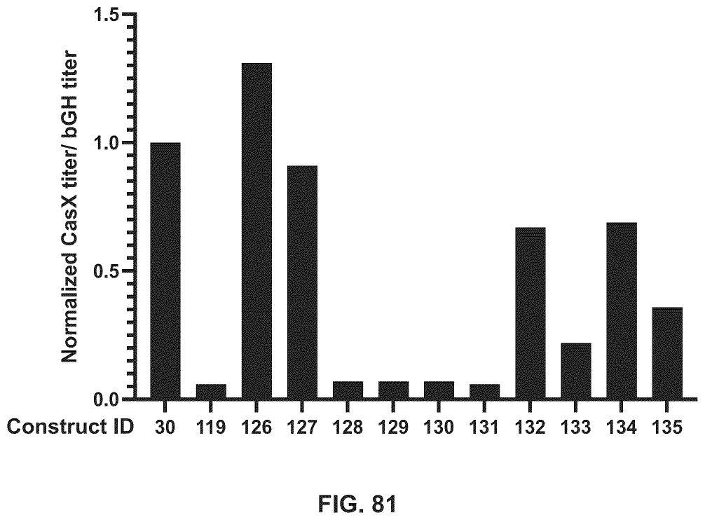

AAV vector production was performed as described in Example 1. In addition to encoding for decoy gRNAs listed in Table 34, the resulting AAVs tested in this example harbored a self-inactivation system where the CasX nuclease construct was flanked on both the 5′ and 3′ ends by a tdTomato proto-spacer sequence preceded by a TTCN PAM. The resulting siAAVs encoded for CasX 491 with gRNA scaffold 174 and spacer 12.7. AAVs without the self-inactivation system or siAAVs without the decoy gRNA were produced and used as experimental controls. Viral genome titer was determined using similar methods as described earlier in Example 1. Here, primer-probe sets were designed to amplify the 3′ region of the CasX locus and the bGH poly(A) signal sequence. The ratio of CasX titer to bGH titer was determined for each condition and normalized to the AAV control with no STALL or decoy gRNA. Experimental conditions tested in this example are outlined below the bar chart in FIG. 81.

### Results:

FIG. 81 illustrates the calculated ratios of CasX titer to bGH titer for the indicated experimental conditions that employed the decoy gRNA strategy for bypassing CasX-mediated cleavage of the AAV transgene during production. The findings show that decoy gRNAs with scaffold 234 or 235 and either a non-targeting or no spacer (construct ID 132-135) were able to restore viral titer when compared to the siAAV titer determined for the scrambled decoy gRNA conditions (construct ID 128 and 129). Interestingly, decoy gRNAs with scaffold 174 were unable to rescue siAAV titer, indicating that enhanced gRNA scaffolds (here, scaffold variants 234 and 235) used for designing decoy gRNAs resulted in more effective binding to CasX to reduce CasX-mediated editing activity.

These experiments demonstrated that using decoy gRNAs to rescue siAAV titer during siAAV production is a viable alternative strategy to minimize CasX-mediated self-cleavage of the AAV transgene without reducing CasX expression. Furthermore, the data show that using an improved gRNA scaffold as part of the decoy gRNA promotes stronger binding with CasX to prevent CasX from editing the STALL sites during production. Future studies may investigate the effects of using a combinatorial strategy involving both shRNAs and decoy gRNAs to reduce CasX-mediated self-cleavage of the AAV transgene during production.

### Example 23: CpG-Depleted AAVs Demonstrate CasX-Mediated Editing In Vitro

Pathogen-associated molecular patterns (PAMPs) such as unmethylated CpG motifs are small molecular motifs conserved within a class of microbes. They are recognized by toll-like receptors (TLRs) and other pattern recognition receptors in eukaryotes and often induce a non-specific immune activation. In the context of gene therapy, therapeutics containing PAMPs are often not as well-tolerated and are rapidly cleared from the patient given the strong immune response triggered, which ultimately leads to reduced therapeutic efficiency. As a result, there is an unmet need for well-tolerated gene therapy vectors that are not cleared rapidly to achieve the necessary therapeutic benefit.

CpG motifs are short single-stranded DNA sequences containing the dinucleotide CG. When these CpG motifs are unmethylated, they act as PAMPs and therefore potently stimulate the immune response. In this example, experiments were performed to deplete CpG motifs in the AAV construct encoding CasX variant 491, guide scaffold variant 235, and spacer 7.37 targeting the endogenous B2M (beta-2-microglobulin) locus and demonstrate that CpG-depleted AAV vectors were able to edit effectively in vitro. The editing activity induced from use of the individual elements of the AAV genome and their respective CpG-reduced versions, as well as stacking of these elements, was assessed in vitro. In vitro assessment of immunogenicity is presented in Example 24.

### Materials and Methods

### Design of CpG-Depleted AAV Plasmids:

Nucleotide substitutions to replace native CpG motifs in AAV components were designed in silico. For exemplary regulatory elements, nucleotide substitutions to replace native CpG motifs were designed based on homologous nucleotide sequences from related species to produce CpG-reduced variants for the following elements: the murine U1a snRNA (small nuclear RNA) gene promoter, the human UbC (polyubiquitin C) gene promoter, and the human U6 promoter. See Table 35, which provides parental sequences of a murine U1a promoter, a human UbC promoter, and a human U6 promoter prior to CpG reduction and Table 36, which provides sequences of CpG-reduced variants of the promoters listed in Table 35. Similar modifications were made to produce a CpG-reduced variant of a bGHpA (bovine growth hormone polyadenylation) sequence. See Table 37, which provides a parental sequence of a bGHpA prior to CpG reduction and Table 38, which provides a sequence of a CpG-reduced variant of the bGHpA listed in Table 37.

AAV2 ITRs were CpG-depleted as previously described (Pan X, Yue Y, Boftsi M. et al., 2021, Rational engineering of a functional CpG-free ITR for AAV gene therapy. Gene Ther.) See Table 39, which provides parental ITR sequences prior to CpG reduction and Table 40, which provides sequences of CpG-reduced variants of the ITRs listed in Table 39.

Nucleotide substitutions to replace native CpG motifs in exemplary Cas protein variants (CasX variants) were rationally designed with codon optimization, so that the amino acid sequence of the CpG-reduced Cas-encoding sequence would be the same as the amino acid sequence of the corresponding native Cas-encoding sequence. See Table 41, which provides parental Cas sequences prior to CpG reduction and Table 42, which provides sequences of CpG-reduced variants of the Cas proteins listed in Table 41. Furthermore, nucleotide substitutions to replace native CpG motifs within the base gRNA scaffold variants (gRNA scaffold 235 and 316) were rationally designed with the intent to preserve editing activity. The rational design process for the CpG reduction of the gRNA sequences is further described herein below. See Table 43, which provides parental gRNA sequences prior to CpG reduction and Table 44, which provides sequences of CpG-reduced variants of the gRNAs listed in Table 43.

All resulting sequences were ordered from a third-party commercial source as synthesized gene fragments with the appropriate overhangs for cloning and isothermal assembly to replace individually the corresponding elements of the existing base AAV plasmid (construct ID 183). Spacer 7.37 (GGCCGAGAUGUCUCGCUCCG; SEQ ID NO: 2709), which targets the endogenous B2M gene, was used for the relevant experiments discussed in this example. The resulting AAV constructs were generated using standard molecular cloning techniques. Cloned and sequence-validated plasmid constructs were midi-prepped for subsequent nucleofection and AAV vector production.

TABLE 35
Parental sequences of promoters

| Parental element | DNA SEQ ID NO: |
|---|---|
| Parental UbC promoter (human) | 464 |
| Parental U1A promoter (murine) | 2930 |
| Parental U6 promoter (human) | 661 |

TABLE 36
Sequences of CpG-reduced or depleted promoters

| | AAV | |
|---|---|---|
| CpG-reduced or depleted | construct | |
| element | ID: | DNA SEQ ID NO: |
| CpG-reduced UbC promoter | 184 | 2904 |
| (human) | | |
| Strongly CpG-reduced UbC | 185 | 2905 |
| promoter (human) | | |
| CpG-depleted UbC promoter | 186 | 2906 |
| (human) | | |
| CpG-reduced U1a promoter | 178, 206 | 2907 |
| (murine) | | |
| CpG-depleted U1a promoter | 179, 205 | 2908 |
| (murine) | | |
| CpG-reduced U6 promoter | 180 | 2909 |
| (human) | | |
| CpG-depleted U6 promoter | 181, 205, 206 | 2910 |
| (human) | | |
| CpG-reduced | — | 2911 |
| hU6 Isoform 2 | | |
| CpG-depleted hU6 Isoform 2 | — | 2912 |
| CpG-depleted hU6 Isoform 3 | — | 2913 |
| CpG-depleted hU6 Isoform 4 | — | 2914 |
| CpG-depleted hU6 Isoform 5 | — | 2915 |

TABLE 37
Parental sequence for Poly(A) signal sequence

| Parental element | DNA SEQ ID NO: |
|---|---|
| Parental bGH-polyA sequence (bovine) | 514 |

TABLE 38
Sequences of CpG-reduced Poly(A) signal sequence

| CpG-reduced or | | |
|---|---|---|
| depleted element | AAV construct ID: | DNA SEQ ID NO: |
| CpG-depleted bGH-polyA | 182, 205, 206 | 2917 |
| sequence (bovine) | | |

TABLE 39
Sequences of parental AAV ITR sequences

| Parental element | DNA SEQ ID NO: |
|---|---|
| 5′ITR | 423 |
| 3′ITR | 424 |

TABLE 40
Sequences of CpG-reduced or depleted AAV ITR sequences

| CpG-reduced or depleted element | DNA SEQ ID NO: |
|---|---|
| CpG-depleted 5′ITR | 2918 |
| CpG-depleted 3′ITR | 2919 |

TABLE 41
Parental sequences of CasX proteins

| Parental element | DNA SEQ ID NO: |
|---|---|
| CasX 491 | 749 |
| CasX 515 | 4015 |
| CasX 676 | 4016 |
| CasX 593 | 4017 |
| CasX 812 | 4018 |
| CasX 668 | 4019 |
| CasX 672 | 4020 |

TABLE 42
CpG-depleted sequences of CasX proteins

| | AAV | |
|---|---|---|
| | construct | |
| CpG-depleted element | ID: | DNA SEQ ID NO: |
| CpG-depleted CasX 491 | 205, 206 | 4021 |
| CpG-depleted CasX 515 | — | 4022 |
| CpG-depleted CasX 593 | — | 4023 |
| CpG-depleted CasX 812 | — | 4024 |
| CpG-depleted CasX 668 | — | 4025 |
| CpG-depleted CasX 676 | — | 4026 |
| CpG-depleted CasX 672 | — | 4027 |

TABLE 43
Parental sequences of gRNA scaffolds

| Parental element | DNA SEQ ID NO: |
|---|---|
| Scaffold 235 | 698 |
| Scaffold 316 | 4028 |

TABLE 44
Sequences of CpG-reduced or depleted gRNA scaffolds.

| | Derived from parent | |
|---|---|---|
| Scaffold ID: | scaffold: | DNA SEQ ID NO: |
| Scaffold 320 | Scaffold 235 | 4029 |
| Scaffold 321 | Scaffold 235 | 4030 |
| Scaffold 322 | Scaffold 235 | 4031 |
| Scaffold 323 | Scaffold 316 | 4032 |
| Scaffold 324 | Scaffold 235 | 4033 |
| Scaffold 325 | Scaffold 235 | 4034 |
| Scaffold 326 | Scaffold 316 | 4035 |
| Scaffold 327 | Scaffold 235 | 4036 |
| Scaffold 328 | Scaffold 235 | 4037 |
| Scaffold 329 | Scaffold 316 | 4038 |
| Scaffold 330 | Scaffold 235 | 4039 |
| Scaffold 331 | Scaffold 235 | 4040 |
| Scaffold 332 | Scaffold 316 | 4041 |
| Scaffold 333 | Scaffold 235 | 4042 |
| Scaffold 334 | Scaffold 235 | 4043 |
| Scaffold 335 | Scaffold 316 | 4044 |
| Scaffold 336 | Scaffold 235 | 4045 |
| Scaffold 337 | Scaffold 235 | 4046 |
| Scaffold 338 | Scaffold 316 | 4047 |
| Scaffold 339 | Scaffold 235 | 4048 |
| Scaffold 340 | Scaffold 235 | 4049 |
| Scaffold 341 | Scaffold 316 | 4050 |

### Design of CpG-Depleted Guide Scaffolds:

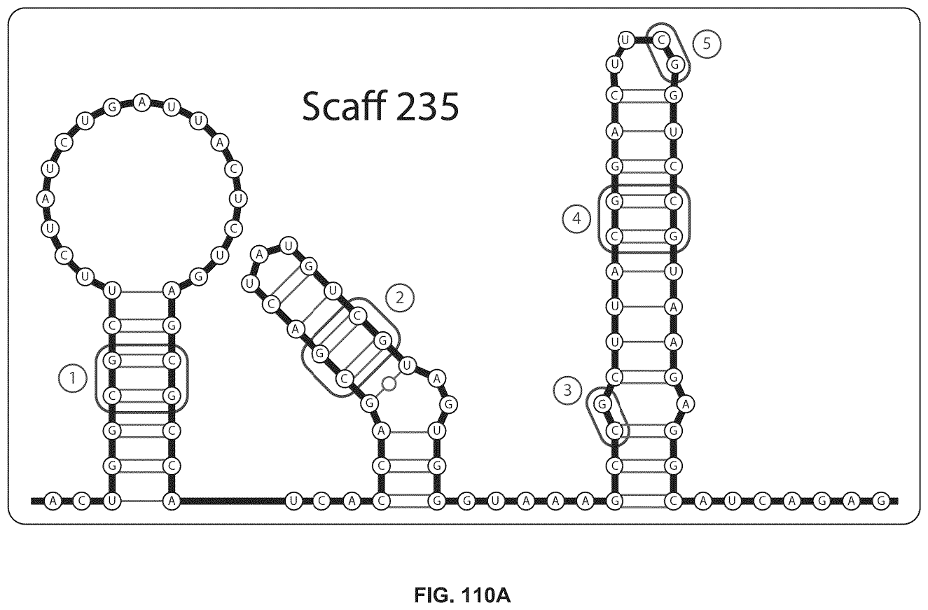

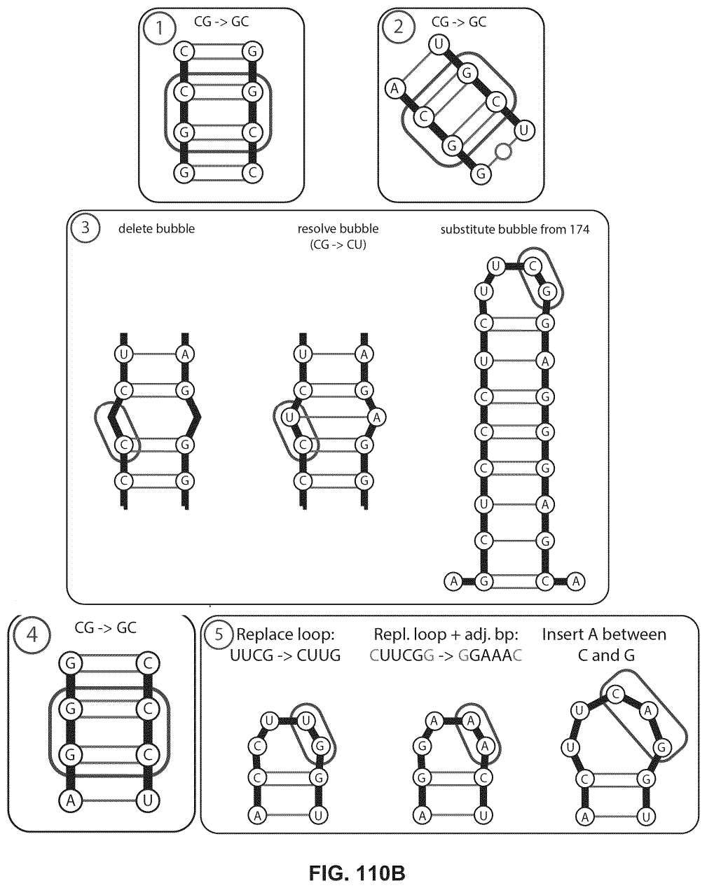

Nucleotide substitutions were rationally-designed to replace native CpG motifs within the base gRNA scaffold variant (gRNA scaffold 235) with the intent to preserve editing activity while reducing scaffold immunogenicity. CpG-motifs were removed from the scaffold coding sequence to reduce immunogenicity. Scaffold 235 contains a total of eight CpG elements; six of which are predicted to basepair and form complementary strands of a double-stranded secondary structure (FIG. 110A). Therefore, the six basepairing CpGs forming three pairs were mutated in concert to maintain these double-stranded secondary structures. These mutations reduced the count of independent CpG-containing regions to five (three CpG pairs and two single CpGs) to be considered independently for CpG-removal. Specifically, mutations were designed in (1) the pseudoknot stem, (2) the scaffold stem, (3) the extended stem bubble, (4) the extended step, and (5) the extended stem loop, as diagrammed in FIG. 110B and described in detail below.

In the pseudoknot stem (region 1), the CpG pair was flipped to a GpC to minimize the alteration of the base composition and sequence. Based on previous experiments involving replacing individual base pairs, it was anticipated that this mutation was not likely to be detrimental to the structure and function of the guide RNA scaffold.

Similarly, in the scaffold stem (region 2) the CpG pair was flipped to a GpC to minimize the alteration of the base composition and sequence. It was anticipated that this mutation was likely to be detrimental to the structure and function of the guide RNA scaffold because strong sequence conservation was seen in this region in previous experiments mutating individual bases or base pairs. This strong sequence conservation is likely due to the scaffold stem loop being important in interacting with the CasX protein as well as in the formation of a triplex structural element with the pseudoknot region.

In the extended stem bubble (region 3) the single CpG was removed by one of three strategies. First, the bubble was deleted by mutating CG→C (removing the guanine from the CpG dinucleotide). Second, the bubble was resolved to restore ideal basepairing by mutating CG→CT (substituting thymine for guanine in the CpG dinucleoide). Third, the entire extended stem loop was replaced with the extended stem loop of scaffold 174. Note that, by itself, the replacement of the extended stem loop with that of scaffold 174 recapitulates scaffold 316, which has previously been shown to edit efficiently. There are no CpG motifs in the extended stem loop of scaffold 174. Therefore, replacing the extended stem loop with that of scaffold 174 also removes the CpG motif in the extended stem (region 4). Based on previous experiments showing the relative robustness of the extended stem to small changes, it was anticipated that mutating the extended stem bubble was moderately likely to be detrimental to the structure and function of the guide RNA scaffold.

In the extended stem (region 4), the CpG pair could not be flipped to GpC without generating additional CpG motifs. Therefore, the CpGs were changed to a GG and a complementary CC motif. Similar to region 3, based on the relative robustness of the extended stem to small changes, it was anticipated that this mutation was not likely to be detrimental to the structure and function of the guide RNA scaffold.

Finally, the extended stem loop (region 5) was mutated in one of three ways that were designed based on previous experiments examining the stability of the stem loop. In particular, several variations of the stem loop had previously been shown to have similar stability levels, and some of these variations of the stem loop do not contain CpGs. Based on these findings, first, the loop was replaced with a new loop with a CUUG sequence. Second, the loop was replaced with a new loop with a GAAA sequence. Since the GAAA loop replacement would generate a novel CpG adjacent to the loop, it was combined with a C→G base swap and the corresponding G→C base swap on the complementary strand, ultimately resulting in a CUUCGG→GGAAAC exchange. Third, the loop was mutated by the insertion of an A to interrupt the CpG motif and thereby increase the size of the loop from 4 to 5 bases. It was anticipated that randomly mutating the extended stem loop would likely have detrimental effects on secondary structure stability and hence on editing. However, relying on previously confirmed sequences was believed to have a lower risk associated with a replacement.

To generate guide RNA scaffolds encoded by DNA with reduced CpG levels, the mutations described above were combined in various configurations. Table 45, below, summarizes combinations of the mutations that were used. In Table 45, a 0 indicates that no mutation was introduced to a given region, a 1, 2, or 3 indicates that a mutation was introduced in that region, as diagrammed in FIG. 110B, and n/a indicates not applicable. Specifically, for region 1, the pseudoknot stem, a 1 indicates that a CG→GC mutation was introduced. For region 2, the scaffold stem, a 1 indicates that a CG→GC mutation was introduced. For region 3, the extended stem bubble, a 1 indicates that the bubble was removed by the deletion of the G and A bases that form the bubble, a 2 indicates that the bubble was resolved by a CG→CT mutation that allows for basepairing between the A and T bases, and a 3 indicates that the extended stem loop was replaced with the extended step loop from guide scaffold 174. For region 4, the extended stem, a 1 indicates that a CG→GC mutation was introduced. For region 5, the extended stem loop, a 1 indicates that the loop was replaced from TTCG to CTTG, a 2 indicates that the loop was replaced along with a basepair adjacent to the loop, from CTTCGG to GGAAAC, and a 3 indicates that an A was inserted between the C and the G.

TABLE 45
Summary of mutations for CpG-reduction
and depletion in guide scaffold 235

| | | | Region 3 | | Region 5 |
|---|---|---|---|---|---|
| | Region 1 | Region 2 | (Extended | Region 4 | (Extended |
| Scaffold | (Pseudoknot | (Scaffold | stem | (Extended | stem |
| ID | stem) | stem) | bubble) | stem) | loop) |
| 320 | 1 | 0 | 0 | 1 | 0 |
| 321 | 1 | 0 | 1 | 1 | 0 |
| 322 | 1 | 0 | 2 | 1 | 0 |
| 323 | 1 | 0 | 3 | n/a | 0 |
| 324 | 1 | 0 | 1 | 1 | 1 |
| 325 | 1 | 0 | 2 | 1 | 1 |
| 326 | 1 | 0 | 3 | n/a | 1 |
| 327 | 1 | 0 | 1 | 1 | 2 |
| 328 | 1 | 0 | 2 | 1 | 2 |
| 329 | 1 | 0 | 3 | n/a | 2 |
| 330 | 1 | 0 | 1 | 1 | 3 |
| 331 | 1 | 0 | 2 | 1 | 3 |
| 332 | 1 | 0 | 3 | n/a | 3 |
| 334 | 1 | 1 | 2 | 1 | 1 |
| 335 | 1 | 1 | 3 | n/a | 1 |
| 336 | 1 | 1 | 1 | 1 | 2 |
| 337 | 1 | 1 | 2 | 1 | 2 |
| 338 | 1 | 1 | 3 | n/a | 2 |
| 339 | 1 | 1 | 1 | 1 | 3 |
| 340 | 1 | 1 | 2 | 1 | 3 |
| 341 | 1 | 1 | 3 | n/a | 3 |
| 235 | 0 | 0 | 0 | 0 | 0 |

Generation of CpG-Depleted AAV Plasmids to Assess CpG-Reduced or Depleted gRNA Scaffolds:

The CpG-reduced or depleted gRNA scaffolds were tested in the context of AAV vectors that were otherwise CpG-depleted, with the exception of the AAV2 ITRs. Specifically, nucleotide substitutions to replace native CpG motifs in AAV components were designed in silico based on homologous nucleotide sequences from related species for the following elements: the murine U1a snRNA (small nuclear RNA) gene promoter, the bGHpA (bovine growth hormone polyadenylation) sequence, and the human U6 promoter. The coding sequence for CasX 491 was codon-optimized for CpG depletion. All resulting sequences (Tables 44 and 46) were ordered as gene fragments with the appropriate overhangs for cloning and isothermal assembly to replace individually the corresponding elements of the existing base AAV plasmid (construct ID 183). Spacer 7.37 (GGCCGAGAUGUCUCGCUCCG; SEQ ID NO: 2709), which targets the endogenous B2M gene, was used for the experiments discussed in this example. The first time that the experiment was performed (“N=1”), a sample with the non-targeting spacer 0.0 was also included as a control (CGAGACGTAATTACGTCTCG).

The resulting AAV constructs were generated using standard molecular cloning techniques. Cloned and sequence-validated plasmid constructs were midi-prepped for subsequent nucleofection and AAV vector production. The sequences of the additional components of AAV constructs, with the exception of sequences encoding the gRNAs (Table 44), are listed in Table 46.

TABLE 46
Sequences of AAV elements (5′-3′ in AAV construct)

| Element | DNA SEQ ID NO: |
|---|---|
| AAV2 5′ ITR | 423 |
| CpG-depleted U1a promoter | 2908 |
| CpG-depleted cMycNLS-CasX491- | 2916 |
| cMycNLS | |
| CpG-depleted bGH-polyA sequence | 2917 |
| CpG-depleted U6 promoter | 2910 |
| AAV2 3′ ITR | 424 |

### AAV Production

Suspension-adapted HEK293T cells, maintained in FreeStyle 293 media, were seeded in 20-30 mL of media at 1.5E6 cells/mL on the day of transfection. Endotoxin-free pAAV plasmids with the transgene flanked by ITR repeats were co-transfected with plasmids supplying the adenoviral helper genes for replication and AAV rep/cap genome using PEI Max (Polysciences) in serum-free Opti-MEM media. Three days later, cultures were centrifuged to separate the supernatant from the cell pellet, and the AAV particles were collected, concentrated, and filtered following standard procedures.

To determine the viral genome (vg) titer, 1 μL from crude lysate viruses was digested with DNase and ProtK, followed by quantitative PCR. 5 μL of digested virus was used in a 25 μL qPCR reaction composed of IDT primetime master mix and a set of primer and 6′FAM/Zen/IBFQ probe (IDT) designed to amplify a 62 bp-fragment located in the AAV2-ITR. An AAV ITR plasmid was used as reference standards to calculate the titer (vg/mL) of viral samples.

Culturing Human Neural Progenitor Cells (hNPCs) In Vitro:

Immortalized hNPCs were cultured in hNPC medium (DMEM/F12 with GlutaMax™ 10 mM HEPES, 1×NEAA, 1×B-27 without vitamin A, 1×N2 supplemented growth factors hFGF and EGF, Pen/Strep, and 2-mercaptoethanol). Prior to testing, cells were lifted with TrypLE, gently resuspended to dissociate neurospheres, quenched with media, spun down, and resuspended in fresh media. Cells were counted and directly used for nucleofection or will be seeded at a density of ˜10,000 cells per well on a 96-well plate coated with PLF (poly-DL-ornithine hydrobromide, laminin, and fibronectin) 48 hours prior to AAV transduction.

Plasmid Nucleofection into Human Neural Progenitor Cells (hNPCs):

AAV plasmids encoding the CasX:gRNA system, with or without CpG depletion of the individual elements of the AAV genome, were nucleofected into hNPCs using the Lonza P3 Primary Cell 96-well Nucleofector Kit. Plasmids were diluted into two concentrations: 50 ng/μL and 25 ng/μL. 5 μL of DNA was mixed with 20 μL of 200,000 hNPCs in the Lonza P3 solution supplemented with 18% V/V P3 supplement. The combined solution was nucleofected using the Lonza 4D Nucleofector System following program EH-100. The nucleofected solution was subsequently quenched with the appropriate culture media and then divided into three wells of a 96-well plate coated with PLF. Seven days post-nucleofection, hNPCs were lifted for B2M protein expression analysis via HLA immunostaining followed by flow cytometry. Subsequently, stacking of individual CpG-depleted elements to create a combined AAV genome with substantial CpG depletion was performed and similarly tested for editing assessment at the B2M locus in vitro.

### Editing Activity Assessment by HLA Immunostaining and Flow Cytometry:

Seven days after nucleofection, AAV-treated hNPCs were lifted with TrypLE. After cell dissociation, staining buffer (3% fetal bovine serum in dPBS) was used for quenching. The dissociated cells were transferred to a round-bottom 96-well plate, followed by centrifugation and resuspension of cell pellets with staining buffer. After another centrifugation, cell pellets were resuspended in staining buffer containing the antibody (BioLegend) that would detect the B2M-dependent HLA protein expressed on the cell surface. After HLA immunostaining, cells were stained with DAPI to label cell nuclei. HLA+ hNPCs were measured using the Attune™ NxT flow cytometer.

Reprogramming of Induced Pluripotent Stem Cells (iPSCs):

Fibroblast cells from a patient were obtained from the Coriell Cell Repository. iPSCs were generated from these lines by episomal reprogramming and genetically engineered to ectopically express Neurogenin 2 (Neurog2) to accelerate neuronal differentiation. Three iPSC clones were selected for downstream experiments.

### Neuronal Cell Culture:

All neuronal cell culture was performed using N2B27-based media. To induce neuronal differentiation, iPSCs were plated in neuronal plating media (N2B27 base media with 1 μg/mL doxycycline, 200 μM L-ascorbic acid, 1 μM dibutyryl cAMP sodium salt, 10 μM CultureOne, 100 ng/ml of BDNF, 100 ng/ml of GDNF). iNs (induced neurons) were dissociated, aliquoted, and frozen for long term storage after three days of differentiation (DIV3). DIV3 iNs were thawed and seeded on a 96-well plate at ˜30,000-50,000 cells per well. iNs were cultured for one week in plating media and thereafter, half-media changes were performed once every week using feeding media (N2B27 base media with 200 μM L-ascorbic acid, 1 μM dibutyryl cAMP sodium salt, 200 ng/ml of BDNF, 200 ng/ml of GDNF).

### AAV Transduction of iNs In Vitro:

24 hours prior to transduction, ˜30,000-50,000 iNs per well were seeded on Matrigel-coated 96-well plates. AAVs expressing the CasX:gRNA system, with or without CpG depletion of the individual elements of the AAV genome, were then diluted in neuronal plating media and added to cells. Cells were transduced at two MOIs (1E3 or 3E3 vg/cell). Seven days post-transduction, iNs were replenished using feeding media. Seven days post-transduction, cells were lifted using lysis buffer, 4-well replicates were pooled per experimental condition, and genomic DNA (gDNA) was harvested and prepared for editing analysis at the B2M locus using next generation sequencing (NGS). Subsequently, combining individual CpG-reduced or CpG-depleted elements to create a combined AAV genome with substantial CpG depletion was performed and similarly tested for editing assessment at the B2M locus in vitro. Experiments assessing the effects of incorporating CpG-depleted gRNA scaffold constructs on editing at the B2M locus in vitro will also be similarly conducted.

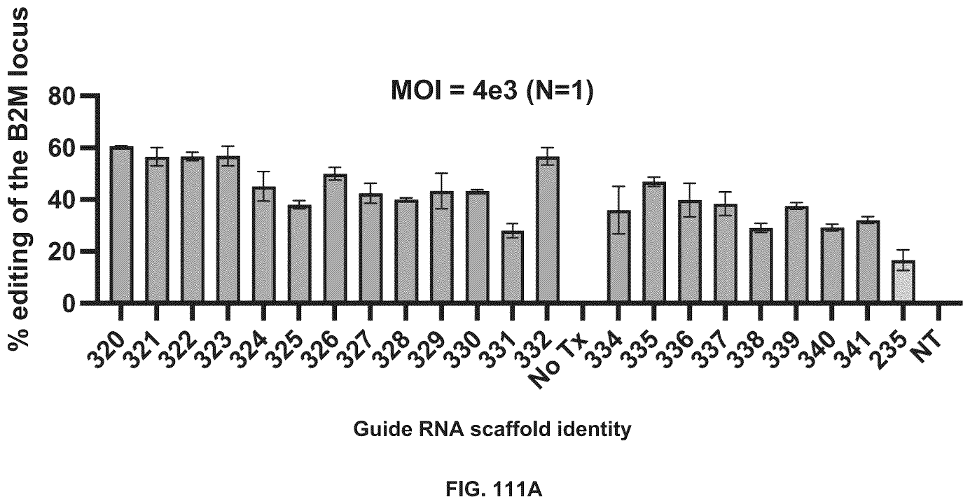

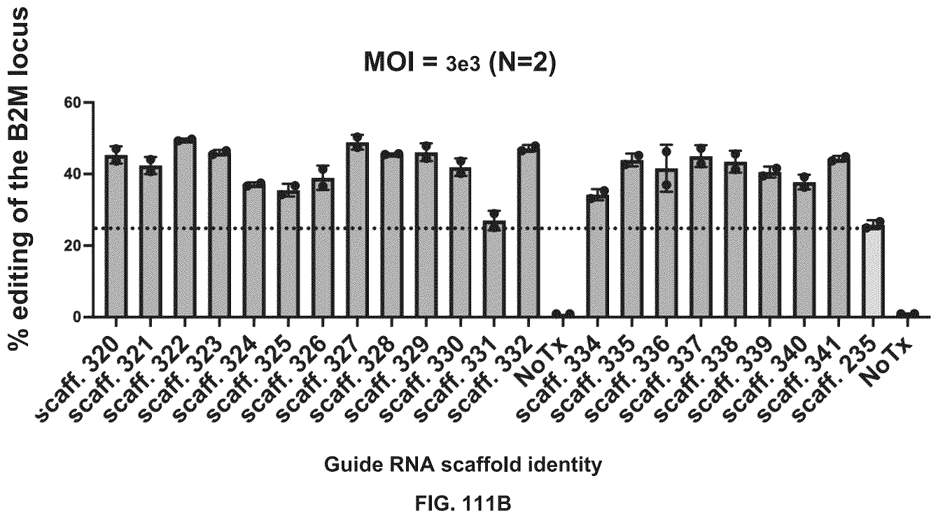

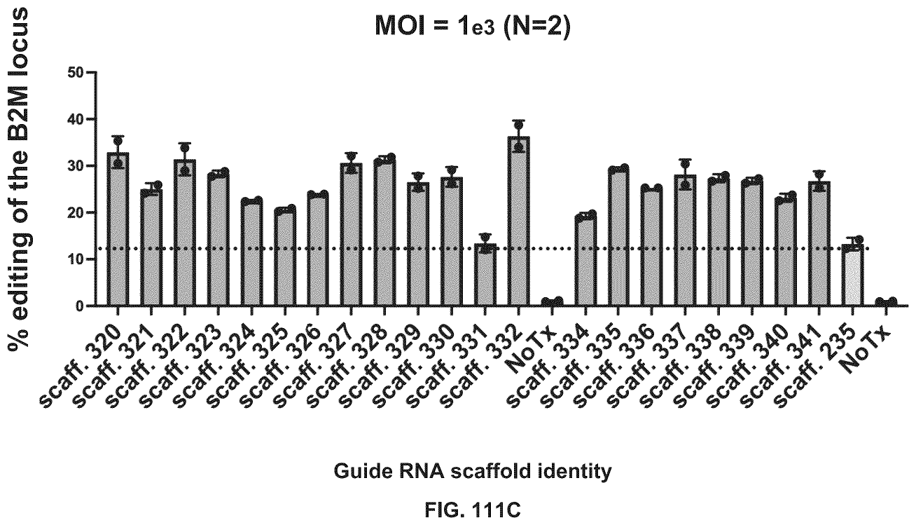

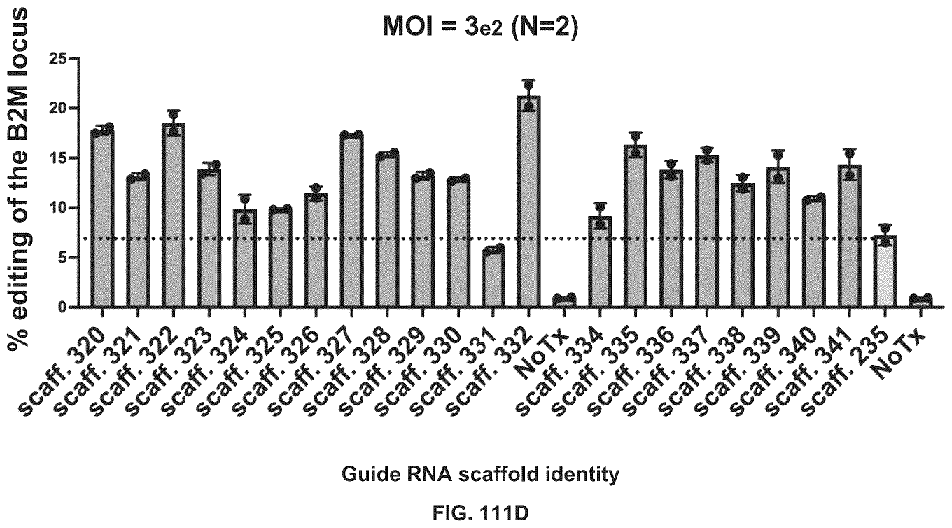

In a separate experiment, CpG-depleted guide scaffolds were assessed. Here, iNs were transduced with AAVs expressing the CasX:gRNA system with various versions of the guide scaffold. The first time that the experiment was performed (“N=1”), cells were transduced at an MOI of 4e3 vg/cell (see FIG. 111A). Seven days post-plating, iNs were transduced with virus diluted in fresh feeding media. Eight days post-transduction, cells were lifted using lysis buffer, 4-well replicates were pooled per experimental condition, and gDNA was harvested and prepared for editing analysis at the B2M locus using NGS. The second time that the experiment was performed (“N=2”), cells were transduced at an MOI of 3e3 vg/cell, 1e3 vg/cell, or 3e2 vg/cell (see FIG. 111B, FIG. 111C, and FIG. 111D. Seven days post-plating, induced neurons were transduced with virus diluted in fresh feeding media. Seven days post-transduction, cells were lifted using lysis buffer, 2-well replicates were pooled per experimental condition, and gDNA was harvested and prepared for editing analysis at the B2M locus using NGS. Samples that were not transduced with AAV were included as controls.

### NGS Processing and Analysis:

Genomic DNA (gDNA) from harvested cells were extracted using the Zymo Quick-DNA™ Miniprep Plus kit following the manufacturer's instructions. Target amplicons were formed by amplifying regions of interest from 200 ng of extracted gDNA with a set of primers specific to the target locus, such as the human B2M gene. These gene-specific primers contained an additional sequence at the 5′ end to introduce an Illumina™ adapter and a 16-nucleotide unique molecule identifier. Amplified DNA products were purified with the Ampure XP DNA cleanup kit. Quality and quantification of the amplicon were assessed using a Fragment Analyzer DNA Analysis kit (Agilent, dsDNA 35-1500 bp). Amplicons were sequenced on the Illumina™ Miseq™ according to the manufacturer's instructions. Raw fastq files from sequencing were quality-controlled and processed using cutadapt v2.1, flash2 v2.2.00, and CRISPResso2 v2.0.29. Each sequence was quantified for containing an insertion or deletion (indel) relative to the reference sequence, in a window around the 3′ end of the spacer (30 bp window centered at −3 bp from 3′ end of spacer). CasX activity was quantified as the total percent of reads that contain insertions, substitutions, and/or deletions anywhere within this window for each sample.

### Results:

### Assessment of Use of CpG-Depleted AAV Vector Elements on Editing in a Cell-Based Assay:

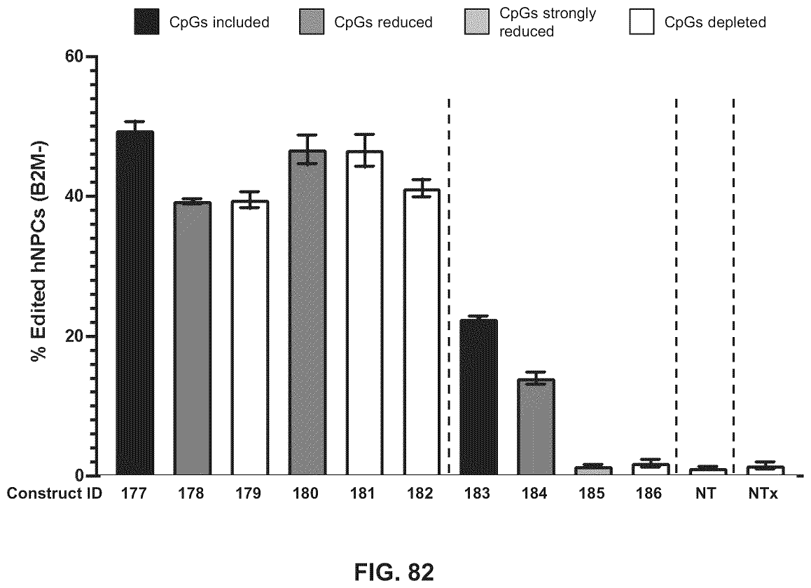

The findings of an assay assessing the editing activity at the B2M locus in hNPCs nucleofected with CpG-containing (CpG+) or CpG-reduced/depleted (CpG⁻) AAV vectors are illustrated in FIG. 82. Editing activity was measured as the percentage of hNPCs that were edited at the B2M locus, resulting in reduced or lack of B2M expression (B2M⁻) on the cell surface. The results shown in FIG. 82 illustrate that reducing or depleting CpG motifs within the sequences of the U1a promoter (construct ID 178 and 179), Pol III U6 promoter (construct ID 180 and 181), or bGH poly(A) (construct ID 182) did not significantly decrease editing activity compared to the editing level achieved with the original CpG⁺ AAV construct (construct ID 177). Specifically, CpG⁻ U1a, CpG⁻ U6, or CpG⁻ bGH resulted in ˜80%, ˜94%, or ˜83% editing of the editing level attained with the base CpG⁺ AAV construct. However, reducing or depleting CpG motifs within the UbC promoter sequence (construct ID 184, 185, and 186) substantially diminished editing activity compared to the level seen with the base UbC construct (construct ID 183), highlighting context-dependent effects of CpG depletion on AAV editing activity and underscoring the importance of screening individual CpG-depleted AAV elements to retain potent editing.

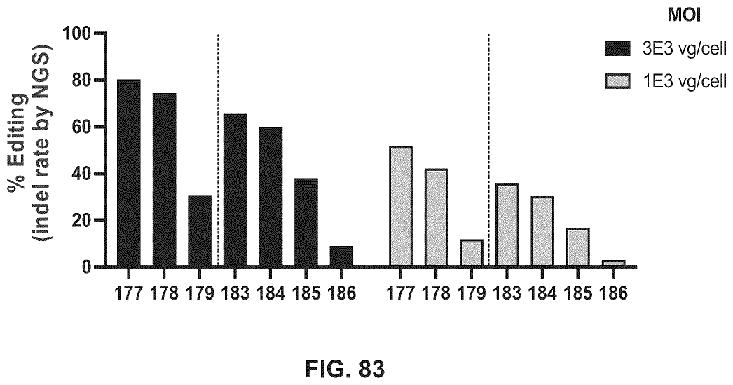

The bar plot in FIG. 83 illustrated that use of the U1a promoter (construct ID 177) resulted in higher editing at the B2M locus when compared to the editing level after use of the UbC promoter (construct ID 183) at both MOIs. This improvement in editing was recapitulated when comparing the use of their CpG-reduced and CpG-depleted counterparts at both MOIs (compare construct ID 178-179 to construct ID 184-186; FIG. 83). Furthermore, depleting CpGs in either U1a or UbC resulted in reduced editing when compared to the editing observed from using their wild-type (WT) or CpG-reduced counterparts (FIG. 83). Interestingly, depleting CpGs in the U1a promoter nevertheless resulted in relatively higher editing compared to the editing level achieved when depleting CpGs in the UbC promoter (FIG. 83).

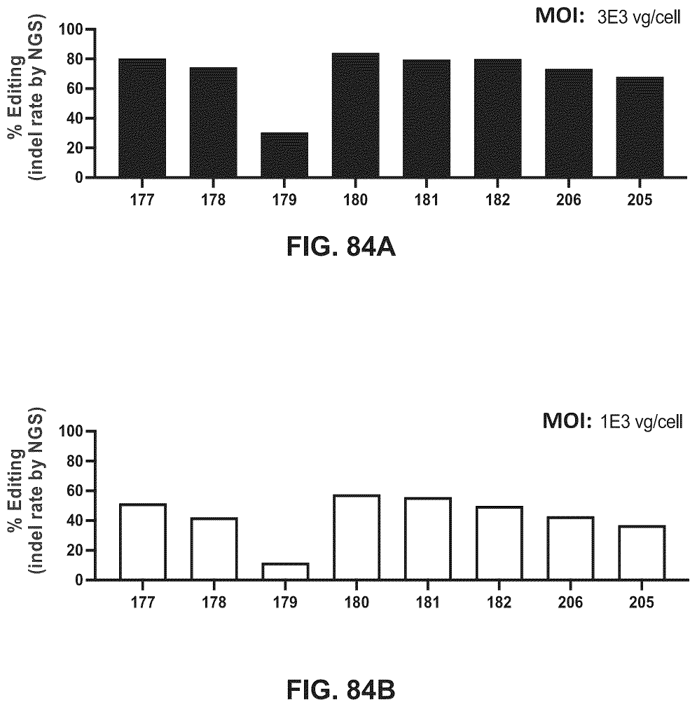

In addition to evaluating the effects of depleting CpGs in different protein promoters (e.g., U1a compared to UbC) on editing mediated by the CasX:gRNA system delivered by AAVs, the effects of depleting CpGs in other elements on editing were analyzed at two MOIs (FIGS. 84A-84B). Furthermore, individual CpG⁻ elements were combined to generate an AAV genome with substantial CpG depletion, and the consequential effects on editing at the B2M locus were assessed (FIGS. 84A-84B).

FIGS. 84A-84B each show bar plots that illustrate the quantification of percent editing at the B2M locus as detected by NGS seven days post-transduction of AAVs into human iNs at an MOI of 3E3 (FIG. 84A) or 1E3 (FIG. 84B). Various CpG-reduced or CpG-depleted AAV elements were tested to assess the effects of their use on editing efficiency at the B2M locus as follows: 177 (no CpG depletion); 178 (U1A promoter with reduced CpG); 179: (U1A promoter with CpG depleted); 180 (U6 promoter with reduced CpG); 181(U6 promoter with CpG depleted); 182 (bGH poly(A) with CpG depleted); 206 (U1A promoter with reduced CpG, CasX491 with CpG depleted, bGH with CpG depleted, and U6 promoter with CpG depleted); 205 (U1A promoter with CpG depleted, CasX491 with CpG depleted, bGH with CpG depleted, and U6 promoter with CpG depleted). ITRs are wild-type sequence.

Several key conclusions were determined from these results illustrated in FIGS. 84A-84B: 1) use of CpG-depleted U1a promoter resulted in a drastic decrease in editing compared to the editing from using the WT or CpG-reduced U1a, supporting findings observed in FIG. 83; 2) depleting CpGs in either the bGH-polyA or U6 RNA promoter resulted in similar editing levels as that achieved by their WT counterpart; and 3) combining CpG-depleted or CpG-reduced elements to build a combined AAV genome with substantial CpG reduction could still retain editing activity (FIGS. 84A-84B).

Additionally, results from experiments aimed to assess the effects of incorporating CpG-depleted gRNA scaffold constructs into a combined AAV genome with substantial CpG depletion on editing at the B2M locus may reveal that varying levels of editing potency can be achieved when delivered and packaged via AAVs.

These experiments demonstrated that using AAV elements with different levels of CpG depletion can result in varying levels of editing mediated by the CasX:gRNA system when packaged and delivered in vitro via AAVs. The data also revealed that depleting CpGs in certain elements could result in similar levels of editing as that achieved when using their WT counterpart. Incorporating CpG-reduced or CpG-depleted elements further expands the inventory of diverse sequences that could be used to build an AAV genome, potentially reducing the risk of recombination during AAV packaging and production.

### Assessment of Use of CpG-Depleted Guide Scaffolds on Editing in a Cell-Based Assay:

Mutations were introduced into the guide scaffold 235 to reduce the CpG content of the DNA sequence coding the guide scaffold. Surprisingly, compared to scaffold 235, all CpG-reduced and CpG-depleted scaffold variants produced higher levels of editing in induced neurons. This was the case with two independent repeats of the experiment (with the results from the first repeat of the experiment shown in FIG. 111A, and the results of the second repeat of the experiment shown in FIGS. 111B-111D), and across multiple MOIs (FIGS. 111B-111D). The enhanced level of editing was surprising because the goal of reducing CpG content was to simply preserve editing activity while reducing immunogenicity. Instead, the mutations enhanced editing activity, rather than merely preserving it.

Notably, scaffold 320 showed a significant increase in potency over scaffold 235. Scaffold 320 includes mutations to only two regions of the scaffold: in the pseudoknot stem and the extended stem (regions 1 and 4). Further, some combinations of mutations produced worse editing than scaffold 320. However, even the CpG-reduced scaffolds that performed worse than scaffold 320, such as scaffolds 331 and 334, performed similarly to or better than scaffold 235.

Based on these results, without wishing to be bound by theory, it is believed that the boost in potency seen in many of the CpG-reduced and CpG-depleted scaffolds is likely caused by one of the mutations present in all CpG-reduced scaffolds (i.e., region 1 and/or 4). Since the mutation to region 4 is not present in the scaffolds with the extended stem loop replacement (i.e., the third mutation to region 3) and these scaffolds show a similar improvement in potency over 235 as 320 did, it is believed that the beneficial effect is likely caused by the mutation in region 1 (pseudoknot stem), which is present in all tested scaffolds. Further experiments will be performed to test the effect of the individual mutations in the pseudoknot stem (region 1) and the extended stem (region 4) separately.

Further, the N=1 data as presented in FIG. 111A indicate that all the new scaffolds carrying the mutation in region 2 (scaffold stem) edited at a slightly lower level than their respective counterparts without this mutation. This suggests that mutating this position in the scaffold stem may have a small deleterious effect on editing potency. This will be examined in additional experiments.

The results described here demonstrate that introducing mutations that reduced the CpG content of the DNA encoding the guide RNA scaffold resulted in improvements in gene editing relative to guide scaffold 235.

### Example 24: CpG-Depleted AAVs Induce Less TLR9-Mediated Immune Response In Vitro

In the preceding example, CpG-reduced and CpG-depleted AAVs were shown to achieve editing at the human B2M locus. Here, experiments will be performed to assess the effects of CpG reduction or CpG depletion on the activation of TLR9-mediated immune response in vitro. Individual elements of the AAV genome and their respective CpG-reduced or CpG-depleted versions will be subjected to in vitro assessment of immunogenicity to identify the optimal CpG-depleted sequences that reduce undesired TLR9 activation and yield potent editing (as demonstrated in Example 23), before being combined to generate an AAV genome with drastically reduced CpG presence for further evaluation.

### Materials and Methods

AAV plasmid cloning, production of AAV vectors, and titering will be performed as described in Example 2. Human TLR9 reporter HEK293 cells (HEK-Bluem hTLR9) will be used for in vitro immunogenicity assessment post-transduction with CpG-containing (CpG⁺) or CpG-depleted (CpG⁻) AAVs.

These HEK-Bluem hTLR9 cells overexpress the human TLR9 gene, as well as a SEAP (secreted embryonic alkaline phosphatase) reporter gene under the control of an NF-κB inducible promoter. SEAP levels in the cell culture medium supernatant, which can be quantified using colorimetric assays, report TLR9 activation.

For this experiment, 5,000 HEK-Bluem hTLR9 cells will be plated in each well of a 96-well plate in DMEM medium with 10% FBS and Pen/Strep. The next day, seeded cells will be transduced with CpG⁺ or CpG⁻ AAVs expressing the CasX:gRNA system. All viral infection conditions will be performed at least in duplicate, with normalized number of viral genomes (vg) among experimental vectors, in a series of three-fold serial dilution of MOI starting with the effective MOI of 1E6 vg/cell. Levels of secreted SEAP in the cell culture medium supernatant will be assessed using the HEK-Blue™ Detection kit at 1, 2, 3, and 4 days post-transduction following the manufacturer's instructions.

The experiments using HEK-Bluem hTLR9 cells to assess TLR9-modulated immune response are expected to show reduced levels of secreted SEAP from cells treated with CpG-AAVs in comparison to levels from cells treated with unmodified CpG⁺ AAVs. Reduced SEAP levels would indicate decreased TLR9-mediated immune activation.

### Example 25: In Vivo Administration of AAV Vectors with or without CpG-Depleted Genomes to Assess the Effects on Inflammatory Cytokine Production and CasX-Mediated Editing

Experiments will be performed to assess the effects of administering AAV vectors with or without CpG-depleted genomes in vivo. Briefly, AAV particles expressing the CasX:gRNA system (with or without CpG depletion) will be administered into C57BL/6J mice. In these experiments, the combined AAV genome with substantial CpG depletion will be used for assessment. After AAV administration, mice will be bled at various time points to collect blood samples. Production of inflammatory cytokines such as IL-10, IL-6, IL-12, and TNF-α will be measured using ELISA and an assay that will assess transgene-specific T cell populations generated against the SIINFEKL peptide.

### Materials and Methods

### Generation of CpG-Depleted AAV Plasmids:

To assess the generation of transgene-specific T cells, a sequence encoding a SIINFEKL peptide will be cloned into an AAV transgene plasmid on the C- and N-terminus of the encoded CasX protein, along with a gRNA with a ROSA26-targeting spacer. The SIINFEKL peptide is an ovalbumin-derived peptide that is well-characterized and has widely available reagents to probe for T cells specific for this peptide epitope.

Production of AAV vectors and determination of viral genome titer will be performed as described earlier in Example 1.

### Measurement of Inflammatory Cytokines to Assess Humoral Immune Activation:

˜1E12 vg AAVs will be injected intravenously or intraperitoneally into C57BL/6J mice. Blood will be drawn daily from the tail vein or saphenous vein for seven days after AAV injection. Collected blood serum will be assessed for the levels of inflammatory cytokines, such as IL-10, IL-6, IL-12, and TNF-α using commercially available ELISA kits according to the manufacturer's recommendations for murine blood samples (Abcam). Briefly, 50 μL of standard, control buffer, and sample will be loaded to the wells of an ELISA plate, pre-coated with a specific antibody to IL-10, IL-6, IL-12, or TNF-α, incubated at room temperature (RT) for two hours, washed, and incubated with horseradish peroxidase enzyme (HRP) for two hours at RT, followed by additional washes. Wells will be treated with TMB ELISA substrate and incubated for 30 minutes at RT in the dark, followed by quenching with H₂SO₄. Absorbance will be measured at 450 nm using a TECAN spectrophotometer with wavelength correction at 570 nm.

### Assessment of Transgene-Specific T Cell Populations:

Ten days after intravenous injection with AAVs, the spleen will be collected from mice, and T cells will be isolated using the EasySep™ Mouse T Cell Isolation kit. Isolated T cells will be incubated with the following: FITC mouse anti-human CD4 antibody (BD Biosciences), APC mouse anti-human CD8 antibody (BD Biosciences), and BV421 ovalbumin SIINFEKL MHC tetramer (Tetramer Shop). The percentage of CD4+ and CD8+ T cells specific to the SIINFEKL MHC tetramer will be quantified using flow cytometry. FITC, APC, and BV421 will be excited by the 488 nm, 561 nm, and 405 nm lasers and signal will be quantified using suitable filter sets.

### Quantification of AAV-Mediated Genome Editing at the ROSA26 Locus:

To demonstrate that CpG⁻ AAVs exhibit enhanced CasX editing activity relative to CpG⁺ AAVs in vivo, AAV particles containing CasX protein 491 with gRNA targeting the ROSA26 locus will be administered intravenously via the facial vein of C57BL/6J mice. Four weeks post-injection, mice will be euthanized, and the liver and/or muscle tissue will be harvested for gDNA extraction using the Zymo Quick DNA/RNA™ miniprep Kit following the manufacturer's instructions. Target amplicons will be amplified from extracted gDNA with a set of primers targeting the mouse ROSA26 locus of interest and processed for NGS as described earlier in Example 23.

In vivo experiments measuring serum inflammatory cytokine levels are expected to show that CpG-depleted AAVs would significantly dampen production of inflammatory cytokines, such as IL-10, IL-6, IL-12, and TNF-α, thereby reducing immunogenicity and toxicity. In addition, CpG-depleted AAVs are likely to cause less TLR9 activation leading to reduced expansion of T cells against the SIINFEKL peptide fused to CasX. Therefore, injections with CpG-depleted AAVs are expected to yield decreased levels of SIINFEKL-specific CD4+ and CD8+ T cells compared to levels from AAV constructs containing CpG elements.

Since CpG-depleted AAVs are likely to cause less humoral immune activation and non-specific inflammation, as well as less T-cell mediated immunity, titers of CasX-reactive antibodies are also expected to be reduced (i.e., lower ELISA signal quantifying CasX antibodies are anticipated).

Finally, editing capabilities of CpG-depleted AAVs will be assessed by harvesting muscle and/or liver tissue for genomic DNA extraction and subjected to NGS to determine editing levels at the ROSA26 locus. Enhanced CasX editing activity at the ROSA26 locus is anticipated with CpG-depleted AAVs, given their expected likelihood to elicit less humoral immune response in vivo.

### Example 26: Demonstration that Combining shRNA Transcriptional Units Results in Increased Silencing of CRISPR Expression in Producing Cells

Experiments were performed to demonstrate that multiple shRNA transcriptional units can be combined in an AAV construct to increase shRNA production, which results in stronger silencing of CasX expression in the AAV-producing cells to hamper CasX-mediated cleavage of the AAV genome during production. FIG. 85 illustrates a general schematic of variations in which the shRNA transcriptional unit can be arranged and combined (or “stacked”) when supplied on the same plasmid as the AAV transgene.

### Materials and Methods

ShRNA sequences were cloned using similar methods as described in Example 17. For stacked shRNA backbones containing Pol III promoters (hU6, 7SK, H1, mU6; see Table 25 for sequences), transgene backbones were digested with the AatII restriction enzyme, and Gibson Assembly was used to clone permutations of shRNA transcriptional units into the bacterial backbone outside the ITRs. This resulted in a plasmid in which the shRNA sequence, or sequences, were included on the same plasmid as the AAV transgene sequence, but were outside of the region between the ITRs that was packaged into the AAV vectors by the packaging cells.

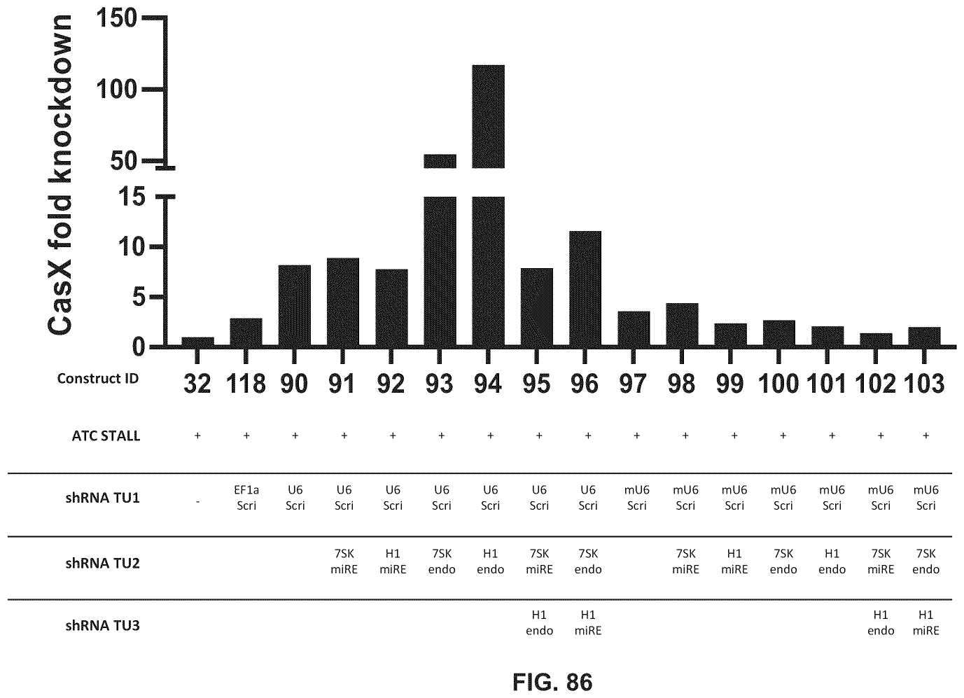

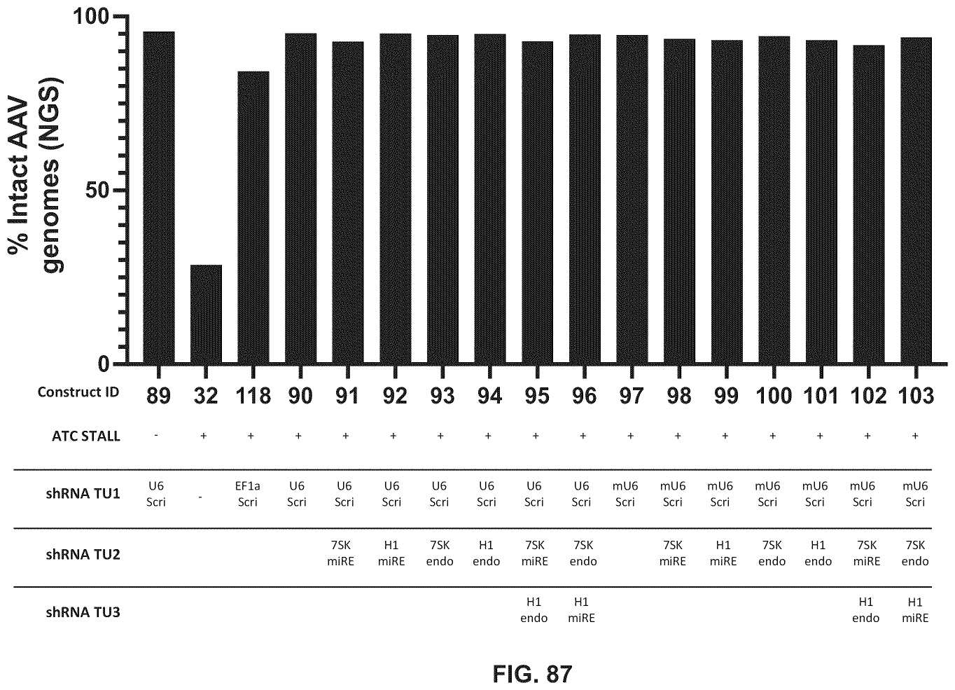

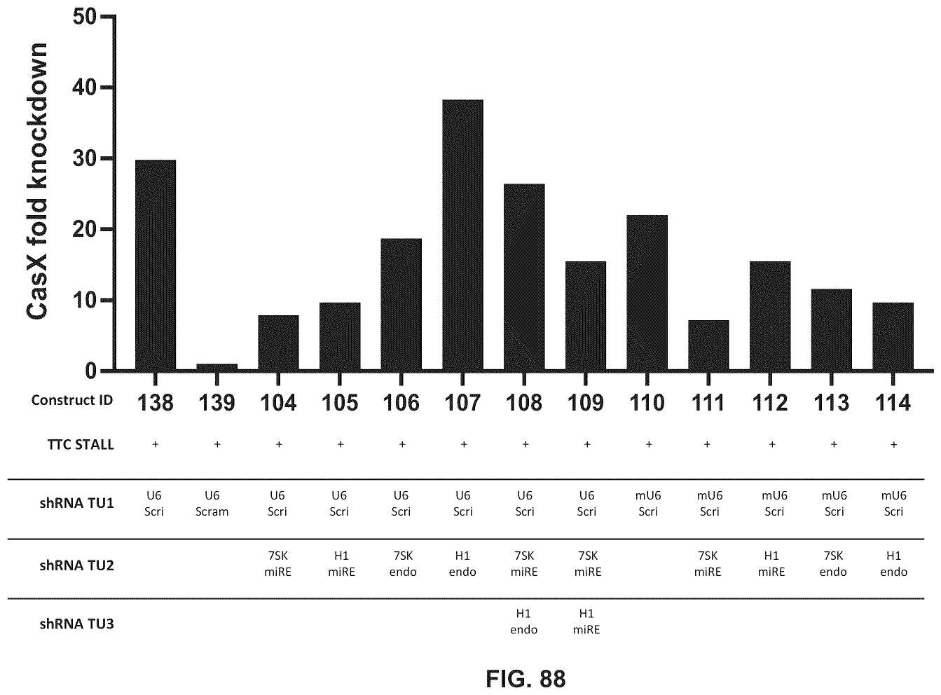

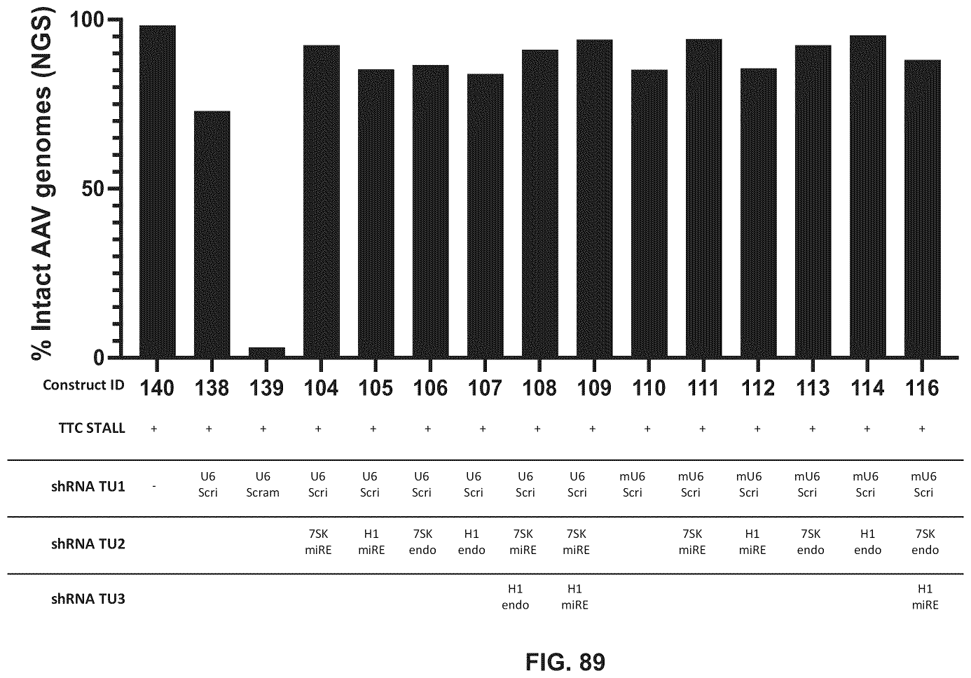

For the constructs used in FIG. 86 and FIG. 87, the AAV transgene encoded for CasX variant 491 that was driven by the UbC promoter, with gRNA scaffold 174; the CasX nuclease construct was flanked on the 5′ end with a tdTomato proto-spacer preceded by an ATCN PAM. For FIG. 88 and FIG. 89, the AAV transgene encoded for CasX variant 491, with gRNA scaffold 174; the CasX nuclease construct was flanked on the 5′ end with a ROSA26 proto-spacer (AGAAGATGGGCGGGAGTCTT (SEQ ID NO: 2920)) preceded by a TTCN PAM.

AAV vector production and titering were performed as described in Example 12. Cell crude lysates were obtained as described in Example 17. Sequences of the various AAV elements are listed in Tables 63-65.

Western blotting was performed as described in Example 17. Western blot quantification was performed using similar methods described in Example 17. The expression of CasX was normalized to total protein to account for loading differences. For FIG. 86, to determine the fold knockdown of CasX, the normalized expression of CasX obtained for construct ID 32 (without an shRNA transcriptional unit) was divided by the normalized expression of CasX obtained for construct ID 90-103 and 118. To determine fold knockdown of CasX shown in FIG. 88, the normalized expression of CasX obtained for construct ID 139 (scrambled shRNA) was divided by the normalized expression of CasX obtained for construct ID 104-114, 116, and 138.

Assessment of ssAAV Genome by NGS:

To assess the presence of edits in the packaged ssAAV genome, ssDNA was isolated from crude lysate or purified viruses by DNase I digest followed by Proteinase K incubation. 1-5 μL of ssDNA was used for amplification of AAV transgene region flanking the self-inactivating on-target spacer. The rest of the protocol was conducted per Example 12.

### Results:

The results in FIG. 86 illustrate that stacking an shRNA8 having a miR-endo scaffold driven either by the 7SK or H1 promoter with a U6-driven shRNA8 having a miR-Scribe scaffold (construct ID 93 or 94 respectively) resulted in dramatically improved knockdown of CasX expression over the knockdown achieved with construct ID 90, which contained a single U6-driven shRNA8 transcriptional unit harboring a miR-Scribe scaffold. Specifically, construct ID 93 and 94 were able to attain a ˜55-fold and ˜117-fold knockdown of CasX expression respectively, while construct ID 90 yielded ˜8-fold knockdown (FIG. 86). Furthermore, stacking a third shRNA transcriptional unit, i.e., H1-miR-endo-shRNA8 (construct ID 95) or H1-miR-E-shRNA8 (construct ID 96) also resulted in CasX knockdown; however, the fold knockdown effects were less substantial compared to that achieved by construct ID 93 or 94 (FIG. 86). Furthermore, use of a mouse U6 (mU6) promoter to drive shRNA8 expression yielded relatively improved CasX knockdown over that achieved when using an EF1α promoter but comparatively less knockdown seen with a human U6 promoter (compare construct ID 97 with 118 and 90 respectively; FIG. 86). The results further show that combining additional shRNA units with the mU6-driven unit does not yield improved knockdowns of CasX (FIG. 86).

NGS analysis of the ssDNA packaged in the siAAV with various iterations of shRNA8 supplementation illustrated and discussed in FIG. 87 revealed that these improvements to the shRNA silencing backbones were all, including the constructs utilizing the mU6 promoter, able to achieve >92% of intact AAV genomes, which was higher than the level achieved (˜84%) with only using the EF1α strategy (construct ID 118).

To differentiate further the functional performance in activity amongst the various shRNA silencing backbone iterations, siAAVs were produced to package an AAV genome containing an alternative TTCN STALL site; previously, the TTCN PAM was shown to demonstrate a relatively higher affinity with CasX compared to that with an ATCN PAM. Findings observed with CasX knockdown in FIG. 86 were recapitulated in FIG. 88: combining an H1-driven shRNA8 with a miR-endo scaffold unit with a hU6-miR-Scribe-shRNA8 unit (construct ID 107) resulted in the highest level of CasX knockdown among the shRNA iterations tested. Furthermore, use of a single hU6-driven shRNA transcriptional unit (construct ID 138) continued to yield a substantial fold knockdown of CasX, especially in comparison to using a mU6 promoter (construct ID 110) or various iterations of a stacked shRNA backbone (construct ID 104-109) to drive shRNA expression (FIG. 88).

The bar graph in FIG. 89 illustrates the quantification of AAV genome intactness as detected by NGS for the same set of constructs assessed in FIG. 88. The results show that inclusion of additional shRNA transcriptional units (construct ID 104-114, and 116) produced more intact AAV genomes compared to the level of intactness achieved by the single hU6-miR-Scribe-shRNA unit (construct ID 138). Specifically, of the various stacked iterations, use of construct ID 114, which contained the combination of mU6-miR-Scribe-shRNA unit with an H1-miR-endo-shRNA unit, produced the highest level of intact AAV genomes at ˜95% out of all the constructs assessed. Furthermore, the data suggest that use of the mU6 promoter is at least as effective in producing intact AAV genomes as use of the hU6 promoter for driving shRNA expression (FIG. 89).

The results from these experiments demonstrate that stacking of shRNA transcriptional units using different combinations of miR-scaffolds and Pol III promoters can result in increased expression of shRNA to silence CasX expression in the producing cells. Additionally, the data demonstrate that various elements of the siAAV vector can be further engineered to achieve greater silencing of CasX-mediated cleavage of the AAV genome during production while simultaneously improving CasX editing at the target locus.

### Example 27: Demonstration that shRNA, or shRNAs, can be Supplied on the pRepCap Production Plasmid to Silence CRISPR Protein Expression in Producing Cells

Experiments were performed to demonstrate that an shRNA, or multiple shRNAs, can be supplied on other plasmids (in this example, the pRepCap plasmid) used for AAV production to reduce CasX protein expression in the producing cell lines. FIG. 72 shows various possible vector arrangements for supplying an shRNA or multiple shRNAs, during packaging, including on the pRepCap or pHelper plasmid.

### Materials and Methods

shRNA sequences were cloned using similar methods as described in Example 17. For pRepCap plasmids containing stacked shRNA silencing elements, the pRepCap plasmid was digested with the NdeI restriction enzyme, and Gibson Assembly was used to clone permutations of shRNA transcriptional units into the bacterial backbone of the pRepCap plasmid. Sequences of the various elements of the tested vectors are listed in Tables 65 and 66.

AAV vectors were produced, and their titer was determined using similar methods described in Example 12, with the exception of using a set of primers specific for the bGH poly(A) signal sequence. Assessment of ssAAV genome by NGS was performed as described in Example 18.

### Results:

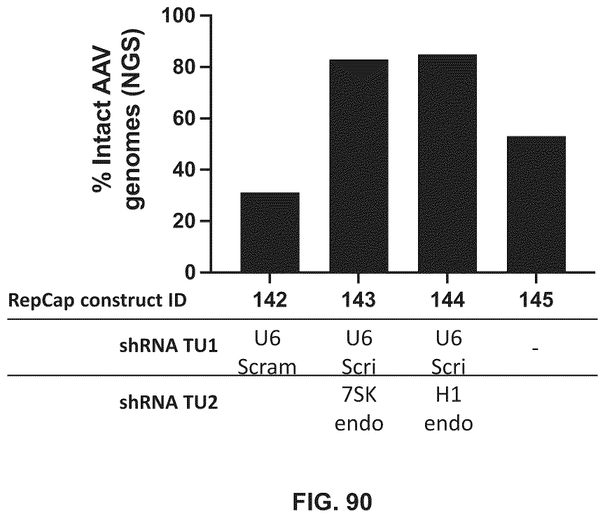

The bar chart in FIG. 90 shows that supplying shRNA or shRNAs to CasX on the pRepCap plasmid resulted in the reduction of CasX-mediated cleavage of the AAV transgene in the packaging cell, and therefore, produced higher levels of intact AAV genomes.

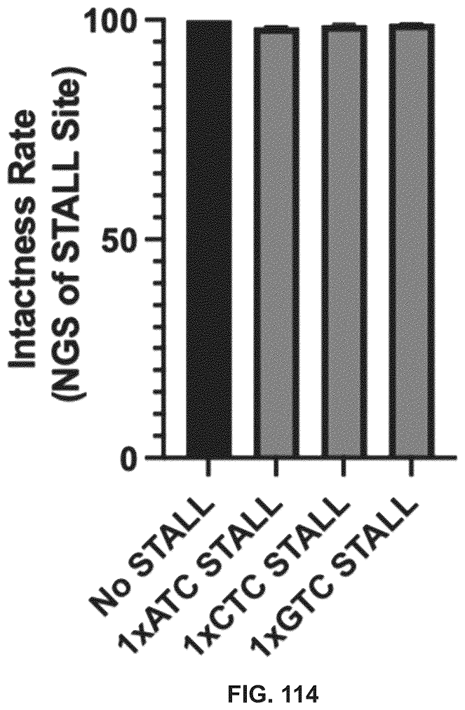

Furthermore, a pRepCap plasmid termed siAAV construct 167 was generated with two shRNA transcriptional units (mU6-shRNA8a and H1-shRNA8b). This pRepCap construct was used to produce AAVs with no STALL site and siAAVs with an ATC, CTC, or GTC STALL site. Assessment of the ssAAV genome by NGS was performed as described in Example 18. As shown in FIG. 114, construct 167 produced high levels of intact siAAVs, supporting the findings in FIG. 90 that supplying shRNAs on the pRepCap plasmid enabled the production of intact AAV genomes. As anticipated, use of construct 167 also resulted in high levels of intact XAAVs.

The results from these experiments demonstrate that expressing an shRNA or multiple shRNAs from production plasmids such as pRepCap is a viable alternative approach to supplying them on the same plasmid as the AAV transgene to silence CasX-mediated premature cleavage of the AAV genome during production.

### Example 28: Guide RNA Guide Scaffold Platform Evolution

Experiments were conducted to identify guide RNA guide scaffold variants that exhibit improved activity for double-stranded DNA (dsDNA) cleavage. In order to accomplish this, a large-scale library of scaffold variants was designed and tested in a pooled manner for functional knockout of a reporter gene in human cells. Scaffold variants leading to improved knockout were determined by sequencing the functional elements within the pool and subsequent computational analysis.

### Materials and Methods

### Library Design

### Assessment of RNA Secondary Structure Stability

RNAfold (v2.4.14) (Lorenz R, et al. ViennaRNA Package 2.0. Algorithms Mol Biol.6:26 (2011)) was used to predict the secondary structure stability of RNA sequences, similar to what was done in Jarmoskaite I., et al. “A quantitative and predictive model for RNA binding by human pumilio proteins”, Mol Cell. 74(5):966 (2019). To assess the ΔΔG_BC value, the ensemble free energy (ΔG) of the unconstrained ensemble was calculated, then the ensemble free energy (ΔG) of the constrained ensemble was calculated. The ΔΔG_BC is the difference between the constrained and unconstrained ΔG values. A constraint string was used that reflects the base-pairing of the pseudoknot stem, scaffold stem, and extended stem, and requires the bases of the triplex to be unpaired.

### Calculation of Pseudoknot Stem Secondary Structure Stability

Pseudoknot structure stability was calculated for the entire stem-loop spanning positions 3-33, using the triplex loop sequence from guide scaffold 175. Further, a constraint string was generated that enforced pairing of the pseudoknot bases and unpairing of the bases in the triplex loop. Changes in stability could thus only be due to the differences in the sequence of the pseudoknot stem. For example, the pseudoknot sequence AAAACG_CGUUUU (SEQ ID NO: 2921) was turned into a stem-loop sequence by inserting the triplex loop sequence CUUUAUCUCAUUACUUUGA (SEQ ID NO: 2922), so that the final sequence would be AAAACGCUUUAUCUCAUUACUUUGACGUUUU (SEQ ID NO: 2923), and the constraint string was: ‘((((((xxxxxxxxxxxxxxxxxxx))))))’ (where x=n).

### Molecular Biology

### Molecular Biology of Library Construction

The designed library of guide RNA scaffold variants was synthesized and obtained from Twist Biosciences, then amplified by PCR with primers specific to the library. These primers amplify additional sequence at the 5′ and 3′ ends of the library to introduce sequence recognition sites for the restriction enzyme SapI. PCR was performed with Q5 DNA Polymerase (New England Biolabs®) and performed according to the manufacturer's instructions. Amplified DNA product was purified with DNA Clean and Concentrator@ kit (Zymo Research). This PCR amplicon, as well as plasmid pKB4, was then digested with the restriction enzyme SapI (New England Biolabs®) and both were independently gel purified by agarose gel electrophoresis followed by gel extraction (Zymo) according to the manufacturer's instructions. Libraries were then ligated using T4 DNA Ligase (New England Biolabs®), purified with DNA Clean and Concentrator® kit (Zymo), and transformed into MegaX DH10B T1R Electrocomp Cells (Thermo Fisher Scientific™) all according to the manufacturer's instructions. Transformed libraries were recovered for one hour in SOC media, then grown overnight at 37° C. with shaking in 5 mL of 2xyt media. Plasmid DNA was then miniprepped from the cultures (QIAGEN®). Plasmid DNA was then further cloned by digestion with restriction enzyme Esp3I (New England Biolabs®), followed by ligation with annealed oligonucleotides possessing complementary single stranded DNA overhangs and the desired spacer sequence for targeting GFP. The oligonucleotides possessed 5′ phosphorylation modifications, and were annealed by heating to 95° C. for 1 min, followed by reduction of the temperature by two degrees per minutes until a final temperature of 25° C. was reached. Ligation was performed as a Golden Gate Assembly Reaction. The reaction was cycled 25 times between 37° C. for 3 minutes and 16° C. for 5 minutes. As above, the library was purified, transformed, grown overnight, and miniprepped. The resulting library of plasmids was then used for the production of lentivirus.

### Library Screening

### LV Production

Lentiviral particles were generated by transfecting LentiX HEK293T cells, seeded 24h prior, at a confluency of 70-90%. Plasmids containing the pooled library were introduced to a second generation lentiviral system containing the packaging and VSV-G envelope plasmids. Viruses were harvested at 36-48h post-transfection. Viral supernatant was filtered using 0.45 μm PES membrane filters and diluted in cell culture media when appropriate, prior to addition to target cells.

72 hours post-filtration, aliquots of lentiviral supernatant were titered by TaqMan™ qPCR. Viral genomic RNA was isolated using a phenol-chloroform extraction (TRIzol™) followed by alcohol precipitation. Quality and quantity of extraction was evaluated by nano-drop reading. Any residual plasmid DNA was then digested with DNase I just prior to cDNA production by Thermo Fisher™ SuperScript IV Reverse Transcriptase. Viral cDNA was subject to serial dilutions through 1:1000 and combined with WPTRE based primers and TaqMan™ Master Mix prior to qPCR by Bio-Rad CFX96. All sample dilutions are added in duplicate and averaged prior to titer calculations against a known, plasmid-based standard curve. Water is always measured as a negative control.

LV Screening (Transduction, Maintenance, Gating, Sorting, gDNA Isolation)

Target reporter cells are passed 24-48h prior to transduction to ensure cellular division occurs. At the point of transduction, the cells were trypsinized, counted, and diluted to appropriate density. Cells were resuspended with no treatment, library- or control-containing neat lentiviral supernatant at a low MOI (0.1-5, by viral genome) to minimize dual lentiviral integrations. The lentiviral-cellular mixtures were seeded at 40-60% confluency prior to incubation at 37° C., 5% CO2. Cells were selected for successful transduction 48h post-transduction with puromycin at 1-3 μg/ml for 4-6 days followed by recovery in HEK or Fb medium.

Post-selection, cells were suspended in 4′6-diamidino-2-phenylindole (DAPI) and phosphate-buffered saline (PBS). Cells were then filtered and sorted on the Sony MA900. Cells were sorted for knockdown of the fluorescent reporter, in addition to gating for single, live cells via standard methods. Sorted cells from the experiment were lysed, and the genome was extracted using a Zymo Quick-DNA™ Miniprep Plus following the manufacturer's protocol.

### Processing for Next Generation Sequencing (NGS)

Genomic DNA was amplified via PCR with primers specific to the guide RNA-encoding DNA, to form a target amplicon. These primers contain additional sequence at the 5′ ends to introduce Illumina™ read and 2 sequences. Amplified DNA product is purified with Ampure XP DNA cleanup kit. A second PCR step was done with indexing adapters to allow multiplexing on the Illumina™ platform, followed by purification, and quality and quantification assessment. Amplicons were sequenced on the Illumina™ Miseq™ according to the manufacturer's instructions.

### NGS Analysis (Sample Processing and Data Analysis)

Reads were trimmed for adapter sequences with cutadapt (version 2.1), and the guide sequence (comprising the scaffold sequence and spacer sequence) was extracted for each read (also using cutadapt v 2.1 linked adapters to extract the sequence between the upstream and downstream amplicon sequence). Unique guide RNA sequences were counted, and then each scaffold sequence was compared to the list of designed sequences and to the sequence of guide scaffolds 174 (SEQ ID NO: 2238) and 175 (SEQ ID NO: 2239) to determine the identity of each.

Read counts for each unique guide RNA sequence were normalized for sequencing depth using mean normalization. Enrichment was calculated for each sequence by dividing the normalized read count in each GFP-sample by the normalized read count in the associated naive sample. For both selections (R2 and R4), the GFP- and naive populations were processed for NGS on three separate days, forming an enrichment value for each scaffold in triplicate. An overall enrichment score per scaffold was calculated after summing the read counts for the naive and GFP-samples across triplicates.

Two enrichment scores from different selections were combined by a weighted average of the individual log 2 enrichment scores, weighted by their relative representations within the naive population.

Error on the log 2 enrichment scores was estimated calculating a 95% confidence interval on the average enrichment score across triplicate samples. These errors are propagated when combining the enrichment values for the two separate selections.

### Results and Discussion

### Library Design, Ordering, and Cloning

A library of guide RNA variants was designed to both test variation to the RNA scaffold in an unbiased manner and in a targeted manner that focused on key modules within the RNA scaffold.

In the unbiased portion of the library, all single nucleotide substitutions, insertions, and deletions were designed to each residue of guide scaffolds 174 (SEQ ID NO: 2238) and 175 (SEQ ID NO: 2239) (˜2800 individual sequences). Double mutants were designed to specifically focus on areas that could possibly be interacting; thus if in the CryoEM structure (PDBid: 6NY2), two residues were involved in a canonical or non-canonical base pairing interaction, or two residues were predicted to pair in the lowest-energy structure predicted by RNAfold (v2.4.14), then the corresponding residues in guide scaffolds 174 and 174 were mutated (including all possible substitutions, insertions, and deletions of both residues). Adjacent residues to these ‘interacting’ residues were also mutated; however for these only substitutions of each of the two residues were included. In the final library, ˜27K sequences were designed with two mutations relative to guide scaffolds 174 or 175.

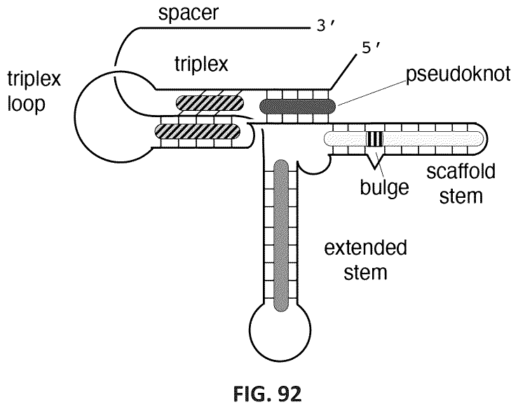

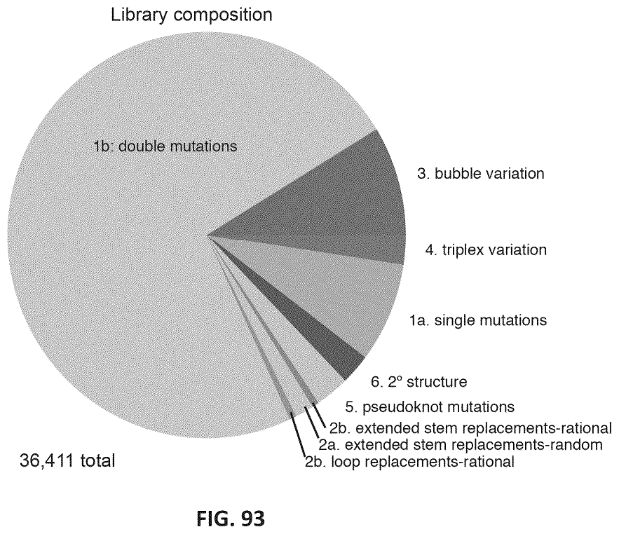

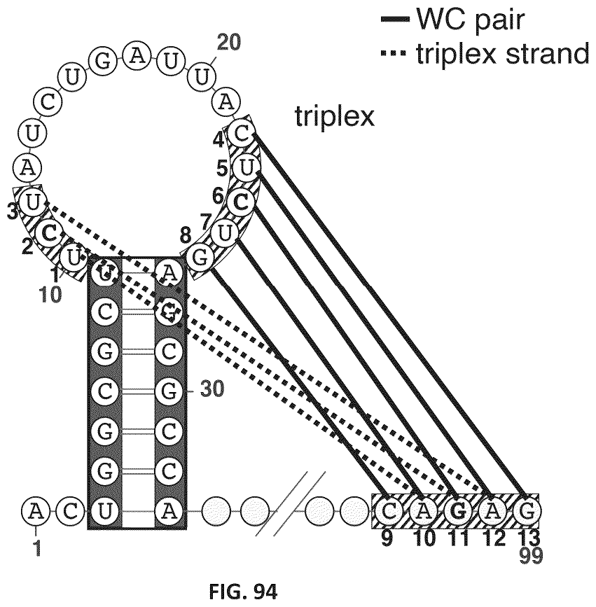

In the portion of the library devoted to specific mutagenesis of key regions of the RNA scaffold, modifications were designed to: the pseudoknot region, the triplex region, the scaffold bubble, and the extended stem (see FIG. 92 for region identification). In each of these targeted sections of the library, the entire domain was mutagenized in a hypothesis-driven manner (FIG. 93). As an example, for the triplex region, each of the base triplets that comprise the triplex was mutagenized to a different triplex-forming motif (see FIG. 94). This type of mutagenesis is distinct from that employed in the scaffold stem bubble, in which all possible substitutions of the bases surrounding the bubble were mutagenized (i.e., with up to 5 mutations relative to guide sequences 174 or 175). In contrast again, the 5 base-pairs comprising the pseudoknot stem were completely replaced with alternate Watson-Crick pairing sequence (up to 10 distinct bases mutagenized).

A final targeted section of the library was meant to optimize for sequences that were more likely to form secondary structures amenable to binding of the protein. In short, the secondary structure stability of a sequence was predicted under two conditions: 1) in the absence of any constraints, 2) constrained such that the key secondary structure elements such as pseudoknot stem, scaffold stem, and extended stem are formed (see Materials and Methods). Our hypothesis was that the difference in stability between these two conditions (called here ΔΔG_BC) would be minimal for sequences that are more amenable to protein binding, and thus we should search for sequences in which this difference is minimal).

The designed library was ordered from Twist (˜40K distinct sequences), and synthesized to include golden gate sites for cloning into a lentiviral plasmid backbone that also expressed the protein STX119 (see Materials and Methods). A spacer sequence targeting the GFP gene was cloned into the library vector, effectively creating single-guide RNAs from each RNA scaffold variant to target the GFP gene. The representation of the designed library variants was assessed with next generation sequencing (see Materials and Methods).

### Library Screening and Assessment

The plasmid library containing the guide RNA variants and a single CasX protein (version 119) was made into lentiviral particles (see Materials and Methods); particles were titered based on copy number of viral genomes using a qPCR assay. A cell line stably expressing GFP was transduced with the lentiviral particle library at a low multiplicity of infection (MOI) to enforce that each cell integrated at most one library member. The cell pool was selected to retain only cells that had a genomic integration. Finally, the cell population was sorted for GFP expression, and a population of GFP negative cells was obtained. These GFP negative cells contained the library members that effectively targeted the CasX RNP to the GFP protein, causing an indel and subsequent loss of function.

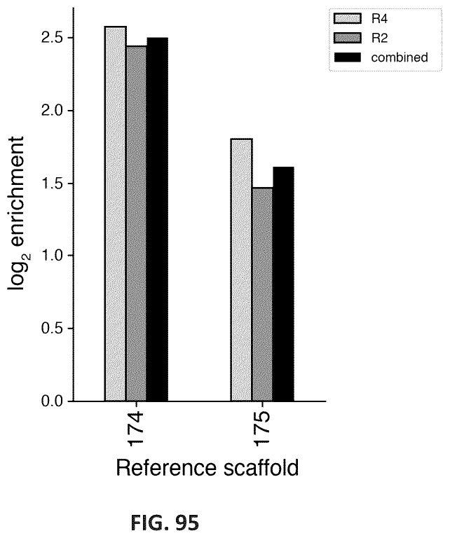

Genomic DNA from the unsorted cell population (“naive”) and the GFP negative population was processed to isolate the sequence of the guide RNA library members in each cell. To determine the representation of guide RNAs in the naive and GFP negative populations, next generation sequencing was performed. Enrichment scores were calculated for each library member by dividing the library member's representation in the GFP-population by its representation in the naive population: A high enrichment score indicates a library member that is much more frequent in the active, GFP negative population than in the starting pool, and thus is an active variant capable of effectively generating an indel within the GFP gene (enrichment value >1, log 2 enrichment >0). A low enrichment score indicates a library member that is depleted in the active GFP-population compared to the naive, and thus ineffective at forming an indel (enrichment value <1, log₂ enrichment <0). As a final statistic for comparison, the relative enrichment value was calculated as the enrichment of a library member (in the GFP negative vs naive population), divided by the enrichment of the reference scaffold sequence (in the GFP negative vs naive population). (In log space, these values are simply subtracted.) The enrichment values of the reference scaffold sequences are shown in FIG. 95).

The screen was performed multiple times, with independent production of lentiviral particles, transduction of cells, selection and sorting to obtain naive and GFP negative populations, and sequencing to learn enrichment values of each library member. These screens were called R2 and R4, and largely reproduce the enrichment values obtained for single nucleotide variants on guide scaffolds 174 and 175 (data not shown). The screen was able to identify many possible combinations of mutations that were enriched in the functional GFP-population, and thus can lead to functional RNPs. In contrast, no guides that contained non-targeting spacers were enriched, confirming that enrichment is a selective cutoff (data not shown). The full set of mutations on guide scaffolds 174 and 175 that were enriched are given in Tables 47 and 48, respectively. These lists reveal the sequence diversity still capable of achieving targeted, functional RNPs.

### Single Nucleotide Mutations Indicate Mutable Regions of the Scaffold:

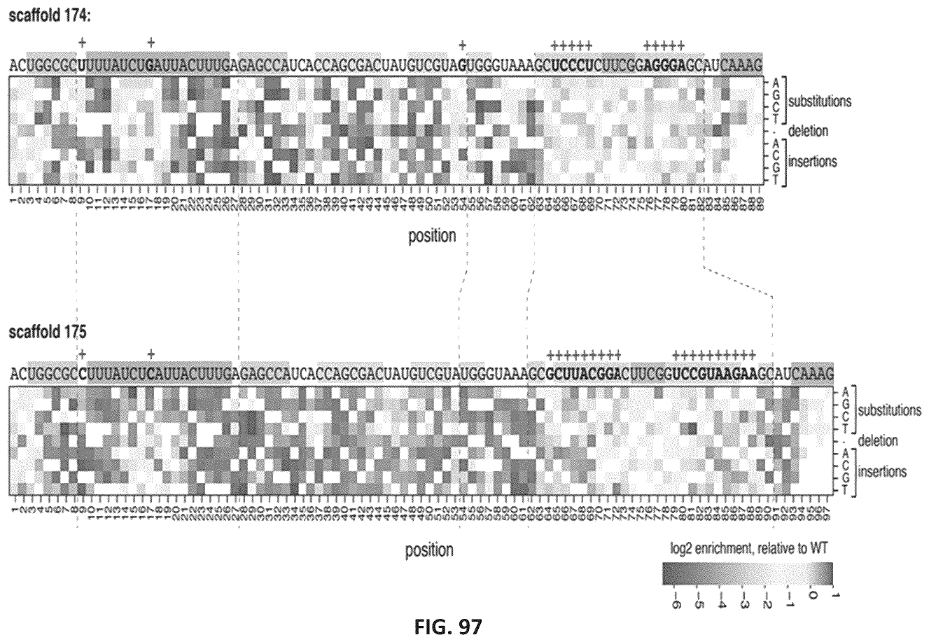

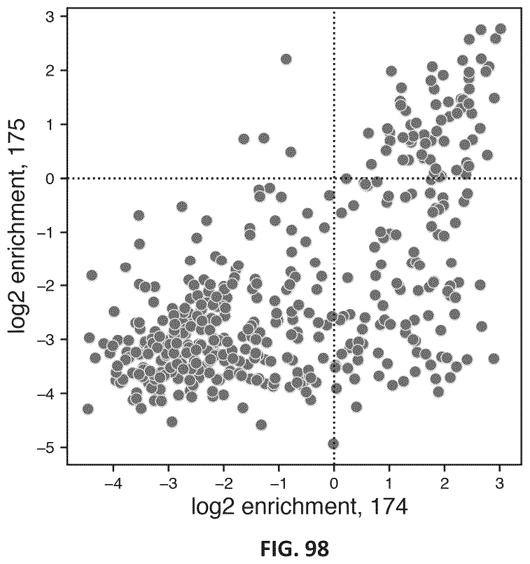

To determine scaffold mutations that lead to similar or improved activity relative to guide scaffolds 174 and 175, enrichment values of single nucleotide substitutions, insertions, or deletions were plotted as heat maps (FIG. 97). Generally, single nucleotide changes on guide scaffold 174 were more tolerated than guide scaffold 175, perhaps reflecting higher activity of guide scaffold 174 in this context and thus a higher tolerance to mutations that dampen activity (FIG. 95 and FIG. 98). Single nucleotide mutations on 175 that were favorable were also favorable in the context of guide scaffold 174 in the vast majority of cases (FIG. 98), and thus the values for mutations on guide scaffold 175 were taken to be a more stringent readout of mutation effects. Key mutable areas were revealed by this analysis, as described in the following paragraphs:

The most notable feature was the extended stem, which showed similar enrichment values as the reference sequences for scaffolds 174 or 175, suggesting that the scaffold could tolerate changes in this region, similar to what has been seen in the past and would be predicted by structural analysis of the CasX RNP in which the extended stem is seen to have little contact with the protein.

The triplex loop was another area that showed high enrichment relative to the reference scaffold, especially when made in guide scaffold 175 (e.g., especially mutations to C15 or C17). Notably, the C17 position in 175 is already mutated to a G in scaffold 174, which is one of the two highly enriched mutations at this position to scaffold 175.

Changes to either member of the predicted pair in the pseudoknot stem between G7 and A29 were both highly enriched relative to the reference, especially in guide scaffold 175. This pair is a noncanonical G:A pairing in both guide scaffolds 174 and 175. The most strongly enriched mutation at these positions were in guide scaffold 175, converting A29 to a C or a T; the first of which would form a canonical Watson-Crick pairing (G7:C29), and the second of which would form a GU wobble pair (G7:U29), both of which may be expected to increase stability of the helix relative to the G:A pair. Converting the G7 to a T was also highly enriched, which would form a canonical pair (U7:A29) at this position. Clearly, these positions favor being more stably paired. In general, the 5′ end was mutable, with few changes leading to de-enrichment.

Finally, the insertion of a C at position 54 in guide scaffold 175 was highly enriched, whereas deletion of either the A or the inserted G at the analogous position in guide scaffold 174 both had similar enrichment values as the reference. Taken together, the guide scaffold may prefer having two nucleotides in this scaffold stem bubble, but it may not be a strong preference. These results are further examined in the sections below.

### Pseudoknot Stem Stability is Integral to Scaffold Activity

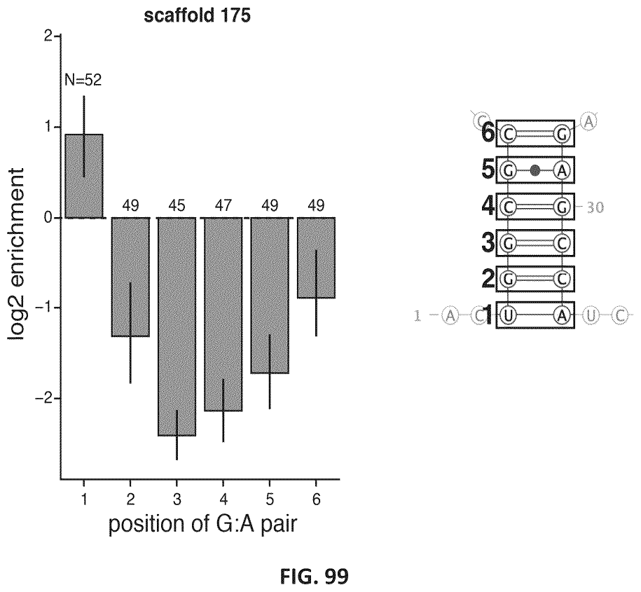

To further explore the effect of the pseudoknot stem on scaffold activity, the pseudoknot stem was modified in the following ways: (1) the base pairs within the stem were shuffled, such that each new pseudoknot has the same composition of base pairs, but in a different order within the stem; (2) the base pairs were completely replaced with random, WC-paired sequence. Two hundred ninety one (291) pseudoknot stems were tested. Analysis of the first set of sequences shows a strong preference for the G-A pair to be in the first position of the pseudoknot stem, relative to the other possible positions (positions 2-6; in the wildtype sequence it is in position 5; FIG. 99), while the results demonstrate that having a GA pair at each of the positions 2-6 in the pseudoknot stem is generally unfavorable, with low average enrichment. Having the G-A bases at position 1 likely stabilizes the pseudoknot stem by allowing the rest of the helix to form from stacking, Watson-Crick pairs only. This result further supports that the scaffold prefers a fully-paired pseudoknot stem.

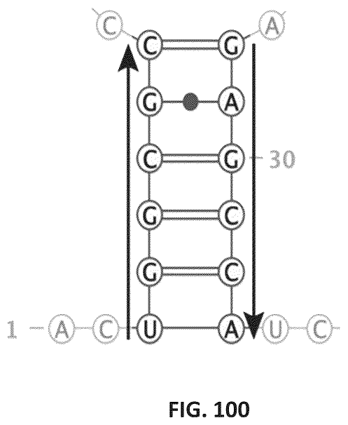

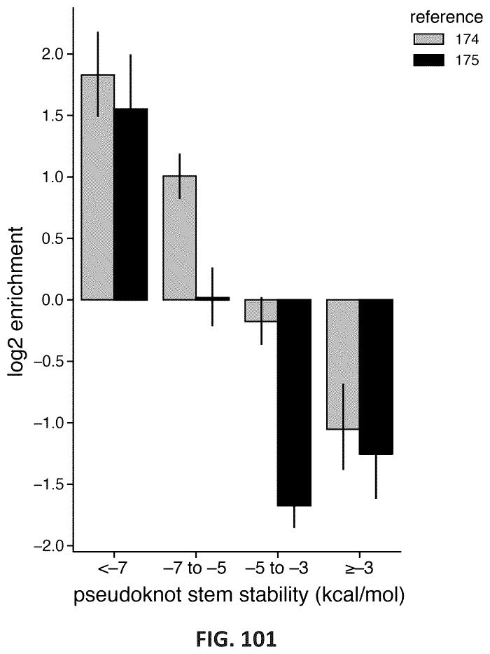

A substantial number of pseudoknot sequences had positive log 2 enrichment, suggesting that replacing this sequence with alternate base pairs was generally tolerated (pseudoknot structure in FIG. 100). To further test the hypothesis that a more stable helix in the pseudoknot stem would result in a more active scaffold, the secondary structure stability of each pseudoknot stem was calculated (Materials and Methods). A strong relationship was observed between pseudoknot stability and enrichment, and thus activity (FIG. 101: more active scaffold have stable pseudoknot stems), with guide scaffolds with stable pseudoknot stems (<−7 kcal/mol) having high enrichment and guide scaffolds with destabilized pseudoknot stems (>−3 kcal/mol) having very low enrichment.

### Double Mutations Indicate Mutable Regions of the Guide Scaffold:

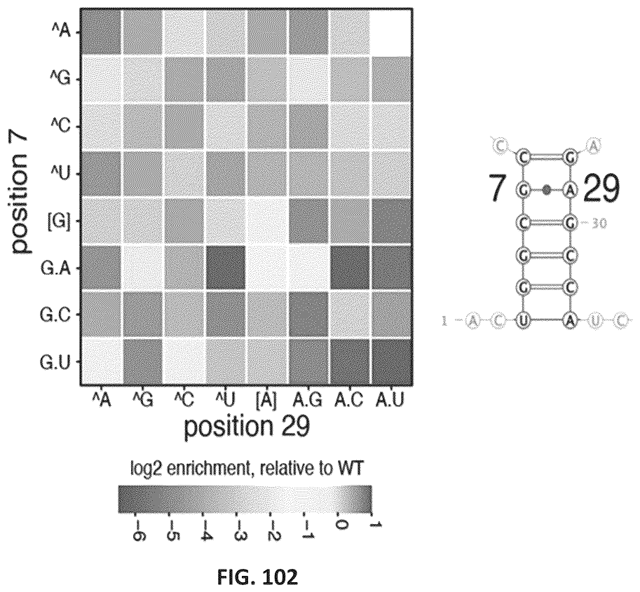

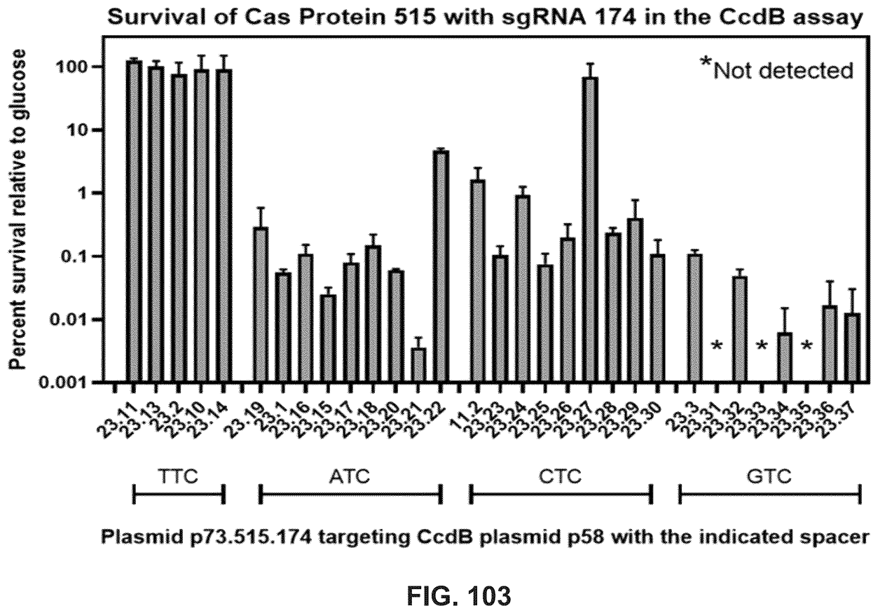

Double mutations to each reference guide scaffold were examined to further identify mutable regions within the scaffold, and potential mutations to improve scaffold activity. Focusing on just a single pair of positions-positions 7 and 29 which are predicted to form a noncanonical G:A pair in the pseudoknot stem and supports mutagenesis (see sections above)—we plot all 64 double mutations for this pair of positions (FIG. 102). Canonical pairs are favored at these two positions (e.g. substitution of a C at position 7 and a G at position 29 creates a G:C pair and is enriched; substitution of a C at position 7 and an insertion of a G at position 29 similarly creates a G:C pair, substitution of an A at position 7 and a U at position 29 creates an A:U pair). No pair of insertions was enriched, perhaps because inserting a canonical pair here is not sufficient to stabilize the helix given that the G:A pair is shifted up a position in the helix and not removed entirely. Surprisingly, several enriched double mutations did not form canonical pairs; e.g. substitutions of U at position 7 and C at position 29 (which forms a noncanonical U:C pair), substitutions of U at position 7 and U at position 29 (forming a U:U pair), as well as a few others (FIG. 103). It is possible that a purine:purine pair is substantially more disruptive to the helix than other noncanonical pairs. Indeed, substitution of an A at position 7 and G at position 29 again forms an A:G pair, which is not enriched at this position.

Enrichment values of double substitutions within each of the key structural elements of guide scaffold 175 were determined from heat maps in which each position could have up to three substitutions. It was determined that the scaffold stem was the least tolerant to mutation, suggesting a tightly constrained sequence in this region.

The results demonstrate substantial changes may be made to the guide scaffold that still result in functional gene knockout when utilized in an editing assay. In particular, the results demonstrate key positions that may be utilized to improve activity through modifications in the guide scaffold, including increased secondary structure stability of the pseudoknot stem within the scaffold.

TABLE 47
Guide 174 mutations and resulting relative enrichment

| Log₂ | |
|---|---|
| enrichment | Mutations on gRNA scaffold 174* (SEQ ID NO: 2238) |
| 3.25 to 3.5 | G79A, A80G; T34A, G78T; G7T, G75A; G78A, A80T; \hat{ }C2, A33T; \hat{ }A1, C68T; |
| | TG3CT, CGC6TAG, GAG28CTA, CA32AG; TG3CA, GC7AA, GA28TT, CA32TG; |
| | \hat{ }C4, C6G, T12_, G17C, GAG28CCC, C32G, A80C; T9C, T14A, T71A, C73A; C70A, G77T |
| 3.0 to 3.25 | A29T, G78T; T9C, G17C, A27T, G79_; C2G, A21G; \hat{ }A81, \hat{ }C81; T71A, C73A; T14C, T16G; |
| | \hat{ }T64, \hat{ }G81; T9C, G17C, \hat{ }TG65; C2G, T16A; G7C, TC14AT, G17A, T34A; G75A, G77A; |
| | G7C, A21T; T-.3.CA, GC.7.-T, G28_, -A.33.TG, \hat{ }T84; T65C, C82T; |
| | GCTCCC63_, \hat{ }AATGAAAA70, \hat{ }TTTTCATT76, GGGAGC77_; \hat{ }C2, G7A, A27T; |
| | T9C, G17C, C67G; \hat{ }A78, \hat{ }T78; T3C, GCG5AGA, AGC29GCT, A33G; T9C, G17C, G78C; |
| | T3C, GC5TG, AGC29CAA, A33G; T9A, \hat{ }T68, G77A; G7A, T9G; T65A, \hat{ }G77; \hat{ }G70, \hat{ }C75; |
| | C2T, G79C; \hat{ }C66, G78A; A29C, G75A; C15A, A60G; C67G, \hat{ }A78; T14C, G17T, G40A, A76G; |
| | T34A, CT64TC; \hat{ }A69, T69A; T45G, G79T; T69C, \hat{ }C76; C2A, G54C; A13C, C15A, G74C; |
| | C70G, \hat{ }A75; A76G, G77C; C67T, G78C; TG3CC, A29C, CA32GG; \hat{ }T7, A29C; C2A, T34A; |
| | \hat{ }A66, \hat{ }A66; C66T, A80C; \hat{ }G17; \hat{ }C76, \hat{ }A76; A29C; C15G, C67G, T72G; \hat{ }T70, \hat{ }A70; |
| | C15G, T16G; C64T, C66A; T69G, G74C; \hat{ }A3, G74C; \hat{ }T65,\hat{ }T80 |
| 2.9 to 3.0 | A29C, A33T; C64T, G78A; \hat{ }C64, A80T; \hat{ }A74; T65A, \hat{ }A80; \hat{ }T69, G75T; \hat{ }C79, \hat{ }A79; |
| | A29G, T59A; T69G, G75C, G78A; \hat{ }G70, \hat{ }A70; G7A, TC14CG, G17A, C64T; \hat{ }T69, \hat{ }A76; |
| | T9C, G17C, C68T, T72C; \hat{ }T69, A76G; A33T, C66G; C66T, C67G; TTC71ACA, \hat{ }GGATGT75; |
| | A13G, T14A; T69A, G74C; G74T, A76G; G77C, G78A; A27C, T84G; C2_, C66G; T71C, G75C; |
| | TC14AG, G78A; T3G, A33T; T9C, G28A; \hat{ }A1, C2T; C68T, T72C; TGGC3CCAG, C8A, GA26_, --A.33.TGG; |
| | C64T, C66G; \hat{ }A67, C67G; C68T, G74A, G77C; G7T; C2T, G78T; C68_, G77T; |
| | T25C, A29C; \hat{ }A78, G78A; \hat{ }C78, G78C; G7C, A60G; T34A, T45A; |
| | T3_, G7A, \hat{ }A9, \hat{ }T28, A29G, A33_; \hat{ }CAG70, T72G, G--.74.AGT; A27G, A29C; T9C, G17C, T47C; |
| | \hat{ }T19; \hat{ }A65, \hat{ }T65; C67G, C68T |
| 2.8 to 2.9 | T3C, G5T, C8G, GA28CC, CCA31AAG; T69C, A76G; C66T, A80T; \hat{ }G13; C2_, T65G; G7C, T9G; |
| | T9C, G17C, TT71AC; C6G, A29T; \hat{ }C66, \hat{ }C79; C70A, A76T; T3A, CG6AC, AG29GT, A33G; |
| | \hat{ }T7, T12_; \hat{ }T69, \hat{ }A76, A88C; C35G, G58C; \hat{ }A79, \hat{ }T79; T16_, C67T, G79_; G7T, T9A; |
| | A29T, C37T, C66G, \hat{ }G77; C2_, G81A; C15G, T34G; T3_, \hat{ }T9, \hat{ }C28, A29C, C32_; \hat{ }T76, \hat{ }A76; |
| | G7C, A27T; C2_, G79C; TGGC3ACAG, GA26_, --A.33.TGT; \hat{ }G65, \hat{ }G77; |
| | \hat{ }AC1, GC5_, C8T, GA26_, G30A, \hat{ }GT34; T9C, G17C, C66T, A80T; T71G, T72G; |
| | G4C, CT8GC, G17C, GA28AC, C32G, T69C, G75C; C41A, G51T; \hat{ }T78; T9C, G17C, T65A, A80C; |
| | AG29CA, C82G; T9G, C82T; T45A, T47C; C2T, T3A; T65A, A80G; C2G, G4A, C32T; G7C, T59G; |
| | T9C, T14G; C2G, A29C, T52A; T9C, G17C, -A.53.CC; T9C, T69_, A76_; C68A, G75C; A1G, A33T; |
| | T3_, \hat{ }T9, G28_, \hat{ }G32; \hat{ }G70, G75C; \hat{ }C54, G54C; \hat{ }T79, G79A; G17C, C70T, A76G; G77A; |
| | \hat{ }T69, A76C; T65A, \hat{ }C80; \hat{ }A66, G79_; T9G, \hat{ }G85; \hat{ }TGGAAGAT63, C---.66.TCGG, |
| | C68A, GGAGGGAG74_, \hat{ }A83; \hat{ }T2; G7A, A29C; \hat{ }A69, \hat{ }C76; C6A, A29C; |
| | C2_, T9C, G17C, GA79TG |
| 2.7 to 2.8 | T34A, \hat{ }T37; A36T, T65C; C2_, T69G; C73A, G74C; G17_; \hat{ }G65, \hat{ }A65; \hat{ }T67, C67T; C2G, A29T; |
| | T9C, G17C, \hat{ }C66, \hat{ }G74; C70A, T71C; T14A, C15T; G4C, C32G, G78C; T9C, G17C, T34A; |
| | \hat{ }A66, \hat{ }C79; AGT53GTG; G79_; T9G, T14A; \hat{ }C64, \hat{ }C80; T65C, \hat{ }G66; \hat{ }GT1, G7A, T9C; |
| | A60T, G78T; T9C, G17C, C67A, G79A; TC65CG, A80G; T14C, T16C; T3_; |
| | \hat{ }CGAAC70, T71A, G74C; G7T, C8G; T3A, GC7CG, GA28CG, A33T; C66T, G78T; |
| | A1G, T9C, G17C; T69C, C70A; C70T, T72G; T69C, T71G, A80T; T16G, A29C; T11G, A29C; |
| | G17A, \hat{ }TA75, A88C; G7T, G40A, A61G; \hat{ }AC81, A88C; \hat{ }A71; G5C, C8G, GA28AC, C31G, C73T; |
| | G74T, A76C; \hat{ }T68, \hat{ }A76; C2_, C70A; T9C, G28T; G28T, A29C; \hat{ }C29; A29C, GA75AC; |
| | \hat{ }T52, G54C; G7A, T9C, G17C; T9C, G17C, G79A; --A.29.CAC; \hat{ }A68, G77A; \hat{ }T69; G7C, T9C; |
| | A80C, C82T; \hat{ }C75, G75T; T14A, A29C; T72C, C73A; T9C, G17C, C66A, G79_; C2_, A33T; |
| | \hat{ }T2, C64T; \hat{ }AT79, A88C; C66G, A80C; \hat{ }A67, \hat{ }T78; \hat{ }G67, G78A; \hat{ }A76; A21G, \hat{ }C66, \hat{ }T77; |
| | C2A, A36G, T69A; G63T, T71C; T9C, G17C, -G.77.CT; \hat{ }T2, T34A; C68T, \hat{ }C77; T9C, G17C, T72C; |
| | T69A, C70T; CT15TA, A18T; TGG3ACA, C8G, GA28CC, CCA31TGT; T9C, A29C; C6G, G30C; -T.3.AA, |
| | C67G; C73_; \hat{ }G68, \hat{ }A76; T69C, \hat{ }A76; A80G; T69C, A76_; \hat{ }G68, \hat{ }T77 |
| 2.6 to 2.7 | T9A, A29C; A76G; T9C, G17C, AG76CC; T9C, \hat{ }A13; \hat{ }A67, \hat{ }T78, A88C; C70A, T72A; |
| | C66G, \hat{ }T79; \hat{ }T64, C64T; \hat{ }A70, C70G; \hat{ }G65, A80C; T9C, G17C, C66T, G78C; C2_, T9G; |
| | T69_, A76T; T3A, G7A, A29T, A33G; T45G, C68A; \hat{ }T65, \hat{ }T80, A88C; C66G; C64T, T71G; |
| | C2G, G54T; A1G, T3A; \hat{ }G70, G75T; T65A, \hat{ }T80; -T.3.AC, |
| | GC.7.A-, GAG28TGC, CA32GG, T72A, \hat{ }G74, A76G; A21C; T69G, \hat{ }A76; C68G, C70A; |
| | C67T, A80T; \hat{ }A70, \hat{ }C75; T9A, T14C; T3A, CGC6TCT, GAG28AGA, A33T; G54_; C68T; |
| | \hat{ }T65, G79C; C2_; C67G, G79T; CT2TG, G7A, G77C; T71G, G74A; C66T, G81A; A29T; --A.29.CAT, |
| | A88C; T69_, \hat{ }C76; T9C, G17C, \hat{ }G68; \hat{ }A69, T69C; A29C, G30T; T69_; G17C; |
| | \hat{ }A67, G78C; T65A; \hat{ }G79, \hat{ }T79; A76G, G77T; \hat{ }GC1, A88C; A27T, A29C; \hat{ }CA79, A88C; |
| | T69_, G75T; C38G, \hat{ }C56, G77A; C68T, G77C; A29C, AG39GT, T52C; G79T, A80T; G7T, A61T; |
| | T16C; \hat{ }A13; G7C, C15G; G5C, C8G, GA28AC, C31G; C2_, G77C; A29C, T52A; G75C, A76G; |
| | T9C, G17C, \hat{ }C76; C8G, A29C; TGG3GTC, C8G, GA28AC, CCA31GAC; |
| | C64_, \hat{ }GTG67, C68A, G77T, \hat{ }CAC79, G81_; \hat{ }T68, G79_; \hat{ }A70, C70A; T65A, AG76GA; |
| | \hat{ }C70, \hat{ }C70; C68G, G77T; C6T, A29T; \hat{ }T81; \hat{ }G67, \hat{ }A67; TGG3GCA, C8A, A29C, CCA31TGC; |
| | G7A, A27C, A29G, A80G; G78A; T52G, G54C; T9C, G17C, T65A, C67T; A1C, \hat{ }C64, \hat{ }T81; |
| | \hat{ }T80, A80C; C67A, C73T; C73T, A80C; C67A, T69C; G7A, A76T, A80G; C2_, C15G; T69C, G77T; |
| | CT2_, G79T; G7C, \hat{ }G28; \hat{ }C79; \hat{ }A80; \hat{ }G1, \hat{ }C1; \hat{ }G65, A80T; G7T, A29_; -T.3.AC, |
| | GC.7.A-, GAG28TGC, CA32GG, T65A; T9C, \hat{ }T14, G17C, \hat{ }C29; A29T, T69C; T9C, A29G; |
| | C64T, T65C; \hat{ }TG70, T71A, C73_, G75T; T65G, C66T; T59C, \hat{ }C66; T72A, G74A; C2T, T72C; |
| | T71C, A76G; T65G, A80T; TG3_, \hat{ }TG7, GA26_, \hat{ }AG33 |
| 2.5 to 2.6 | T9C, G17C, \hat{ }G81; --A.29.CAT; C68T, A76G; A29C, G79A; G17C, C67G, C70T; \hat{ }G66, C66G; |
| | A29T, G63A, C66A; G28C, A29C; T3G, C67A; T69C, T71C; T3A, GC7CA, GA28TG, A33G; |
| | C70G, G74A; \hat{ }C2, G4C, C8_, \hat{ }CGC28, CCA31_; C2_, C68T; C66A, A80T; |
| | T3A, G5C, GC7AA, GA28TT, C31G, A33T; T9C, G17C, T72G; T9C, G17C, A29C; \hat{ }C70, G75T; |
| | C66T; C66T, G78A; A36T, G54C, C68T; \hat{ }G9, A29T; A76C; T69C, G77C; \hat{ }A77, \hat{ }G77; |
| | T71G, G74C; C67T; C73G; T71G, AG76GA; \hat{ }C64, T65C; T3G, C68A; G74C; C67T, T69A; \hat{ }A69; |
| | \hat{ }A66, C66T; T71C; T14G, T16C; T9C, G79T; T65C; \hat{ }C15, G17C; \hat{ }T65, \hat{ }C79; C70G, T71G; |
| | G74C, G75T; C2_, C68G; G7T, A27G; \hat{ }CA76, A88C; \hat{ }T65, \hat{ }A65; T9C, G17C, T45A; A18C, \hat{ }A66; |
| | A80C; G7C, TC14CT, G17A; TG3GC, G7A, A29T, CA32GC; T16G, A29C, G63T, T71C; |
| | C2A, G54T, T71C; \hat{ }T8, A29C; T9C, TG16GC; C70T, G77T; G75T, A76G; T69A; T16A, A18G; |
| | G77A, G78C; A1_, T59C; T14G, T16G; \hat{ }A60, G81A; A29G, A83G; T34A, GA79TC; T69C, G75A; |
| | G7T, T59A; G7T, C82G; A36T, G81T; C2_, G81T; T14C, T72_; --A.29.CAC, A88C; |
| | TGG3_, \hat{ }AAG9, GA28CT, C31G, A33G; G17C, A18G; C66G, G77A; |
| | \hat{ }C5, C6T, C8_, G28C, GC30CG; C82T; G54A, \hat{ }G56; C2_, C66T; G17C, A18C; G17C, G54_; |
| | G28A, T65C; C6T, A29C; G7A, T9C, \hat{ }T79; T9C, GA17CT; G74A, G75T; C68A, C70G; |
| | G42C, C50G; \hat{ }C70, \hat{ }C75; \hat{ }T66, \hat{ }C66; T3C, CGC6GCT, G28_, \hat{ }A32, A33G; C73A, G74A; |
| | TG3AC, C6A, AG29CT, CA32GG; C67A, G79A; A76_; C73G, G74T; |
| | TG3CA, GC7AG, GA28CG, CA32TG; T9C, T14C, T71A, C73A; G81C; A1G, T16A; T69A, \hat{ }G74; |
| | C68_; C2A, A60C; T9C, G54T; T14C, C15G; \hat{ }G66, \hat{ }G66; T16C, A18G; \hat{ }G68, G77C; A29T, -G.78.CC; |
| | G7T, \hat{ }T61; CT2_, T72G; A1G; T65C, C66A; G7C, T34A; \hat{ }C35, T59G; \hat{ }AG77, A88C; |
| | \hat{ }TG67, A88C; |
| 2.4 to 2.5 | G54C, T59A; T69G, G75C; C68A, A76G; \hat{ }AT65, A88C; C68T, G77T; G7T, A29C; |
| | T65A, T71A, G74A; T16A; \hat{ }C65, \hat{ }A65; \hat{ }T67, G79_; \hat{ }G71; \hat{ }C18; \hat{ }C29, A29T; G79A; |
| | T69G, T71A; T71C, T72C; C2_, T3_; \hat{ }T67, G78T; |
| | CTCCCTCT64_, C73G, AGG76TTC, \hat{ }TCCCA82; T65A, A83G; C70A, G74A; |
| | G7C, TC14AT, G17A, T34C; G7T, A33C, A36C, A76G; T-.3.CA, GC.7.A-, AG29GC, CA32TG; |
| | C2_, A80G; -T.3.AC, C6T, C8_, G28C, G30C, CA32GT; G7C, A83G; C2_, C67A; |
| | T3G, A29C, T34G, G77A; C2G, A21G, T65C; G40A, T59A; \hat{ }A66, \hat{ }G66; G81A; C2_, A29G; |
| | \hat{ }T64, G81A; \hat{ }CGC2, CGC6_, GAG28_, \hat{ }AGG33; \hat{ }C77; T69A, A76G; \hat{ }T78, \hat{ }T78; C66A, \hat{ }C79; |
| | C2_, G7A, T34A; T3C, C6T, G30A, A33G, \hat{ }C55; GC7CG, GA28AG; |
| | T3C, G5C, GC7TA, G28T, C31G, A33G; \hat{ }T68, \hat{ }C77; \hat{ }T77, G77A; A27G, \hat{ }GT77; \hat{ }G66, \hat{ }T79, A88C; |
| | T9C, \hat{ }G69, \hat{ }A76; C68T, G75C; \hat{ }T81, \hat{ }T81; \hat{ }C66; T9C, G28C; T14A, A29C, C66T; \hat{ }A65; |
| | T3A, G5C, C8A, A29C, C31G, A33T; CT2_, T71A; G7C, C15G, A33T; G77A, \hat{ }T78; G63T, C82A; |
| | G7A, C15G, G54A, A60C, G79T; \hat{ }A13, \hat{ }G13; T72G, C73T; A36C, G54T; T3G, G7T; \hat{ }G65, T65C; |
| | T65G, C66G; G77C; T45G; C15A; C41T, G51A; T14A; C2T, G54T; A76T; T71A, A76G; |
| | \hat{ }G66, \hat{ }T79; \hat{ }A7, A29C; TGG3AAC, C8G, GA28CC, CCA31GTG; \hat{ }A1; \hat{ }T29; T71G, G74T; T45A; |
| | \hat{ }AT78, A88C; \hat{ }A3, GG4CC, C8_, GA28CG, CCA31GAT; C66T, C70G; C2_, \hat{ }A66, \hat{ }C79; |
| | \hat{ }TA76, A88C; TG3GA, CG6GA, AG29TC, CA32TC; \hat{ }C80, A80G; G79C; C67G, \hat{ }G77; |
| | \hat{ }C66, G79A; G7A, T16C, \hat{ }T68; G7C, T9C, G17C, G75C; C2_, \hat{ }T58; \hat{ }A65, \hat{ }C80; A1G, -C.68.GA; |
| | G17C, T65G; TG3CC, C6T, C8G, GAG28ACA, CA32GG; T72C, G75A; C64G, A80T; G7A, C66T; |
| | C66G, \hat{ }C79; C15A, G17A; \hat{ }AG66, A88C; A36_; G79T; T9C, G17C, \hat{ }T58; T10G, A29T; |
| | \hat{ }G69, \hat{ }C76; \hat{ }A69, A76_; G7A, A29G; A53_; T65G, \hat{ }A80; C70A, C73A; T59C, G74T; C67A; |
| | G54T, \hat{ }G56; \hat{ }G66; C2_, A29C; C38_, G54_; T3_, C6T, \hat{ }C8, \hat{ }G28, G30A, A33_; -TG.3.ACC, |
| | C6A, C8_, \hat{ }CTC28, A29G, CCA31_; T9C, T14A; C64_; T14G, G54A; |
| | T71C, C73G, A83C; T9C, G17C; A53G, G54T; C66A, A80G; \hat{ }G63, \hat{ }G81; \hat{ }G1; \hat{ }C78, \hat{ }C78 |
| 2.3 to 2.4 | G7A, T9C; \hat{ }T67, \hat{ }G67; C2_, C67T; A80_; \hat{ }G1, A13C; \hat{ }G66, G79C; T69A, A76T; T9C, T14C; |
| | A76G, G78C; T16G, G17T; T69C, G77A; T65_, A80G; G7C, T14C, T34G; C66T, C67T; A53G, -A.80.TC; |
| | C67T, G77C; C73A, G74T; A36G, C68G; T9C, G17C, \hat{ }C78; |
| | TGG3GCT, C8G, \hat{ }AC28, CA32_; \hat{ }T18; \hat{ }C29, \hat{ }T29; |
| | \hat{ }GGGCG63, C68T, TTCGGA71_, \hat{ }CCGCC82; T9C, G17C, C66T, A80G; \hat{ }A67, \hat{ }G67; C2_, G79T; |
| | T3A, CGC6GAT, GAG28ATC, A33T; C2_, A21G, G79C; C2_, A21G; C64T, G77A; C8A, G79C; |
| | C67G, \hat{ }A78, A80C; T69C, \hat{ }A70; G74T, G75C; \hat{ }T76, A76G; A76T, A80G; \hat{ }C64; \hat{ }C29, C50T; |
| | \hat{ }AGCTTA65, \hat{ }ATTG68, T69A, T72C, G77T, GA--.79.AGCT; A29T, G30A; T65C, A80C; |
| | \hat{ }C76, \hat{ }T76; T9C; \hat{ }G67, G79_; C68T, G79T; \hat{ }CTCA3, GCGC5_, \hat{ }CAT28, CCA31_; \hat{ }GA70, |
| | A88C; -T.3.AC, GC.7.A-, GAG28TGC, CA32GG; A21G; \hat{ }G69, T69C; G7A, \hat{ }C66, \hat{ }G74; \hat{ }T65, \hat{ }A79; |
| | T65G; G74A, A76C; G74T, G75A; \hat{ }G68, G77T; T9G, G79T; \hat{ }AG67, A88C; \hat{ }C81; \hat{ }A67, G78T; |
| | C37A, G57T; G54C, G79T; G75T, G77A; G40A, TAG52CCT; \hat{ }G15; C67A, C68A; A36T, \hat{ }C55; |
| | A36T, T59A, T65C; C67T, \hat{ }G68; T71C, A76C; G7C, A29G, T65A; \hat{ }A78; T69C, G75T; |
| | \hat{ }TC66, A88C; CT2_, T59A; T9C, G17C, T65G; C70G, G75T; C2_, C73T, G75A; |
| | TG3CC, C6G, C8T, G28A, G30C, CA32GG; C64G, C66T; T11C, A29C; T9C, \hat{ }G15, G17C, T65C; |
| | T69G, G74T; \hat{ }GA65, A88C; G7C, A61G; \hat{ }T65, \hat{ }A80; C68_, \hat{ }C79; G7A, \hat{ }T29, G79T; A27T; |
| | A1_, T9G, T59C; T14G, -G.79.TT; T14C, T16A; C70A, G74T; T65A, G78A; \hat{ }T65, \hat{ }G77; |
| | T9C, G17C, \hat{ }G68, G77C; C66A, \hat{ }A79; G7T, T9C, G17C; \hat{ }G69, A76T; C2_, A21C; \hat{ }T29, A29T; |
| | \hat{ }G69, \hat{ }T69; C6T, T10C, T84G; T65C, C67T; C15T; G78C; G7T, A27G, C44T; \hat{ }C68, \hat{ }A68; |
| | A1G, T9C, G17C, A76G; A36T, T59A; T14A, T16A; \hat{ }C66, G79_; -T.3.AA, G7_, AG29GC, CA32TG; |
| | C8G, \hat{ }A70, \hat{ }T75; C66A; \hat{ }C64, A80_; T69C; T71G, A76T; CT68TC, G74A; G54C, C68T; |
| | T9C, G17C, G81T; C2_, A13G; T65A, \hat{ }C81; \hat{ }C66, \hat{ }A78; \hat{ }C70, \hat{ }A75; \hat{ }T68, G77T; |
| | A29T, C50T, A53G, G79T; C68T, A76T; T16C, A18T, A80C; \hat{ }TGGAAGAT63, C---.66.TCGG, |
| | C68A, GGAGGGAG74_, \hat{ }A83, A86C; -T.3.AA, G7_, AG29GC, CA32TT; T9G, A29G; |
| | C68A; A27C, A29T; A36T, G54C; \hat{ }A4; \hat{ }A73 |
| 2.2 to 2.3 | \hat{ }C66, A76_; \hat{ }G65; \hat{ }T1, T59C; A36T; T3C, GCG5CGA, AGC29TCG, A33G; |
| | T9C, TG16GC, C68T, G79A; G7A, T14A, G17A, T34A; T65G, G79T; G7C, TC14CT, G17A, T34A; |
| | T3C, C67A; G77C, G78T; C2T, \hat{ }G56; C6T, A83G; G7T, C8A; C66G, G79_; TG3_, C--.8.TCG, |
| | \hat{ }C28, \hat{ }C30, CA32_; C67T, T69G; CT2_, T9C; G78T, G79A; C2_, T9C, C15A, G17C; |
| | T9C, G17C, \hat{ }TG67; G75T, A76C; \hat{ }C76; G79A, A80T; TT71GG, G74C; C70_, G75C; \hat{ }G66, G79T; |
| | T34A, A60G; A29T, C64T, C66A; \hat{ }CT29, A88C; \hat{ }G69; A53C, G79T; \hat{ }T80, A80G; \hat{ }G67, C67A; |
| | C67A, G78C; T9C, G17C, C70T, T72A; -T.3.AC, GC.7.A-, A29_, A-.33.GT; C2G, \hat{ }T58; |
| | A27G, \hat{ }A70; A39G, G78C; -G.78.AA, A80C; C66G, C67A; \hat{ }G68, \hat{ }A68; T69C, T71A; |
| | G7T, G40C, AG53GA; T9C, G17C, G79T; C8A, C66T; G74T, A80C; G7C, T14G; \hat{ }C77, G77T; |
| | G58T, G79C; T14C; \hat{ }T65, \hat{ }A80, A88C; C68A, \hat{ }C77; GC--.63.ATTA, CCC66ATT, G-- |
| | GG.77.AATAT, GC81AT; T11G, A29T; T14A, T16G; T71C, G75A; \hat{ }T67, \hat{ }C78; T65C, G81A; |
| | G79C, A80T; \hat{ }C66, \hat{ }G74; A53C, G54A; \hat{ }C66, \hat{ }C79, A88C; G79C, A80G; |
| | T9C, G17C, \hat{ }C66, \hat{ }G74, A88C; C2_, T16C; T69G; \hat{ }G68, \hat{ }A76, A88C; T71A, G74C; G74T; |
| | G7T, C37A; \hat{ }CA68, A88C; \hat{ }T12, \hat{ }G12; A29T, C64T, C70T; G7C, A29G; G7A, T14A; T69C, C70G; |
| | G79T, A80C; C2T, G54C; \hat{ }T58; G7T, G30A, G81T; A29C, A83G; C2_, T69C; |
| | T3C, G5C, G7C, A29G, C31G, A33G; T72G; C64A; T34G, T59C; A1G, A60C; T65A, G79A; |
| | A27T, \hat{ }C29; \hat{ }G67, \hat{ }G77; \hat{ }G68, C68A; C64G; C66T, G77A; \hat{ }C64, \hat{ }A80; C2_, C73T; A29G; \hat{ }T7; |
| | A1_, A46C, T59C; T9C, G17C, A76T; G78C, A80G; \hat{ }C66, A76C; \hat{ }T29, \hat{ }T29; |
| | A27T, CT68TC, G74A; G75C, A76C; \hat{ }TT81, A88C; \hat{ }G77, A80G; \hat{ }C5, G7T; \hat{ }C66, T69C; |
| | C15A, T16A; C73T; \hat{ }A65, \hat{ }A80; \hat{ }T65, G79_; G40A, T52C; G7T, A60T; |
| | TG3GA, GC7CA, A29G, CA32TC; \hat{ }TA70, A88C; \hat{ }C66, \hat{ }A66; \hat{ }G67; A36C, \hat{ }T55, C68T; T65_; |
| | G63_, C82_; C2A, A29G |
| 2.1 to 2.2 | A83G; G75_; C68_, G79_; C2_, A46C; \hat{ }C4; \hat{ }A69, \hat{ }A69; G42A, C50T; A53G, \hat{ }T55; A36G, \hat{ }C58; |
| | TG3AC, C8A, GA28TC, CA32GG, T59C, C66A; C2_, A46C, C66T; C64T, G81T; \hat{ }A68, G77T; |
| | \hat{ }T80, A80T; T25G, A29T; G4A, C32T, G54_; \hat{ }T68; A76C, G78A; T9C, T14C, G17C; CT2_, A33C; |
| | \hat{ }CA65, A88C; A60C; \hat{ }A69, \hat{ }T69; T9C, G17C, -T.65.GC; A18C, A61G, A80C; CT15TG, A21C; |
| | T72G, A76T; G7C, A29C; \hat{ }G79, \hat{ }C79; T69G, \hat{ }T76; C70A, G74C; T9G, A29C; C2_, G54A; |
| | C15G, T72A, G74A; \hat{ }A75; T3_, C6T, \hat{ }C8, A29_, C32A, \hat{ }C34; \hat{ }C29, A80C; G74A, A76T; |
| | C68T, T69C; T3_, C64T; A80T; CT2_, T9A; \hat{ }C29, A36C; \hat{ }GA67, A88C; T9C, G17C, T59A; |
| | A60T, C64T; T65A, G79T; A29C, T65C; \hat{ }T7, A13C; C8A, C82T; A76G, \hat{ }C77; |
| | T3G, GC7CT, GA28AG, CA32AC; ---TT.71.AAGAA, G75_; G7T, C15G; \hat{ }C79, \hat{ }C79; |
| | TG3GA, CG6AC, A29G, CA32TC, C68T, T72C; T72C; G63C, C82T; \hat{ }TG56, G57T; |
| | T14C, A29T, A36T; \hat{ }T68, \hat{ }T68; T69G, T71G; \hat{ }G66, C66T; \hat{ }G68, G77A; G54C, G79A; G7T, C67G; |
| | C66G, G78A; A60C, A76G, A80G; G40A, -A.76.CC; C2T, C67A, \hat{ }T78; T9C, G17C, G77A, G79T; |
| | G77T, G78A; \hat{ }T78, \hat{ }C78; \hat{ }T68, G77C; \hat{ }A67, \hat{ }G77; C73T, G75A; A29T, C66A, G74T; C2G, A36G; |
| | T3G, G5A, GC7CA, A29G, C31T, A33C; T69A, T71C; \hat{ }CG2, G5_, C8_, --G.28.CGC, CA32_; |
| | \hat{ }GT79, A88C; C68A, G77T; C64T; G40A, G77C; C68G, C70G; C2T, G78A; |
| | T9C, G17C, \hat{ }C66, A76C; G7T, A29G, C82T; C2_, T65G, A80G; TGG3GCT, C8G, \hat{ }CC28, CC31_; |
| | A29G, T69C, A80G; T34A, A36_; T9C, G17C, A27G; C15T, T16C; |
| | G7T, T9C, G17C, G40A, TA52AT; A36G, T71A; C6T; \hat{ }G69, A76_; C66A, G79A; \hat{ }C68, \hat{ }T68; |
| | A21T, C67A; A21C, T72G, G77T; T71G, A76G; C2T, G54A; T71G, G77A; |
| | T9C, G17C, A29G, G81A; G7A, A36T, G54C, C68T; T3A, T59A; \hat{ }G70; \hat{ }T77; \hat{ }T68, \hat{ }C77, A88C; |
| | TC14GT, T72C; T9C, G17C, T72_; \hat{ }C73; G7C, T14C; A36T, \hat{ }T58; G54T; T59C; |
| | A29C, C50T, A60T; G54A, C70G, \hat{ }T75; \hat{ }C66, G77C; C15G, G17C; C64G, \hat{ }C81; |
| | T3A, G5C, GC7AG, GA28CT, C31G, A33G; A29C, C32A; \hat{ }G28; A21G, A53G; G75A, A76T; |
| | G7C, TC14CT, G17C, T34A; G28A |

*mutated sequences are ‘;’-separated and multiple mutations per sequence are ‘,’-separated

TABLE 48
Guide 175 mutations and resulting relative enrichment

| Log₂ | |
|---|---|
| enrichment | Mutations on scaffold 175* (SEQ ID NO: 2239) |
| 3.2 to 3.5 | C73A, \hat{ }T78; C6T, A29C, G71C, \hat{ }G80 |
| 3.1 to 3.2 | C17G, A87C; T3G, CGC6ACT, GAG28AGT, A33C; G7T, C9T, C17G, CG81GA; T16G, A29C; |
| | C9T, C17G, C65A, A87G |
| 3.0 to 3.1 | A68T, T83G; A27G, T92C; TGG3ATC, GC7AG, GA28CT, CCA31GAT; \hat{ }C65, A87G; G7T, A29T; |
| | T3G, GC7AA, GA28TT, A33C; C9T, C17G, C65_; G7T, T14G; \hat{ }G54, G78T; C9T, C17G, \hat{ }A80; |
| | TC16AT, G64C |
| 2.9 to 3.0 | C15T, T34A; C9T, C17G, A88T; G7A, C15G; \hat{ }C76, \hat{ }G76; CT2_, C15_, T58A; C2_, C15G; |
| | C9T, A29C; C9T, C17G, A85T, A88T; C9T, C17G, \hat{ }CA63; G7T, C9G; A87T, A88C; C73G, G78A; |
| | A29T, A91G; TG3GA, G7A, A29G, CA32TC; \hat{ }G14, A29T, A87G; C9T, C17G, T74C; C2_, \hat{ }A53 |
| 2.8 to 2.9 | C9T, A33T; G7T, T67G, G82C; \hat{ }T5, C9_, GAGC28CGCA; G7T, \hat{ }A68, \hat{ }A82; G7T, \hat{ }C60; |
| | T14G, A29C; A29T, T66A; T3A, CG6TC, AG29GA, A33T; C2T, TC75AT; \hat{ }CG76, A88C; |
| | G7T, T14A, T83_; -T.3.GA, C6T, C9_, G28C, G30C, CA32TC; CT2_, C15T; |
| | TG3_, \hat{ }GT8, G30C, C32G; T14_, A29C; C9G, C17G, A29C, T79G; |
| | TG3AC, G7C, A29G, CA32GT, G86C, A88C; T3A, GC7CA, A29G, A33T; G7C, C80A |
| 2.7 to 2.8 | G7T, A91C; \hat{ }C2, G4C, G7_, A29_, C32G, \hat{ }G34; CT2_, A88C; C65G, A88C; G7T, -T.79.AA; A29C; |
| | T3A, GC7CA, A29G; C8G, A29C, A88_; A29T; C2_, A29C; A29C, C31T, A33G; T14G, C15T; |
| | C9T, C15A; \hat{ }GA1, G7A, C15A, C17G; C15A, T16A; CT2_, A29C; C9T, C17G, G78_; |
| | C9T, C17G, G-.78.AT; C73T, C76G |
| 2.6 to 2.7 | C9T, C17G, C65_, \hat{ }A84; C9T, C17G, G70T, C81A; T74A, T79A; T3C, C6T, AG29CA, A33G; |
| | G7A, \hat{ }T29; C76G, G77C; GG77CA, A87G; T16G, A29T; T3A, G5A, A29C, C31T, A33G; |
| | C9T, C17G, \hat{ }AA53; TG3CA, GC7AA, GA28TT, CA32TG; G7A, A29C; T3G, G7T; CT2_, A68G; |
| | T14_, A29T; C2_, C9T, C17G; \hat{ }G3, GC.7.-T, G28_, \hat{ }C34; G7T, \hat{ }T92; G7T, \hat{ }G69, G82T; |
| | \hat{ }GGCAGATCTGA64, T66C, A68C, GA71AG, \hat{ }C75, G77T, T79C, CGTAAGAA81_; |
| | T3A, C6G, AG29CC, A33T; C80T, \hat{ }A81; C81T; CT2_, C17A; C15A, T16G; C2_, T16G; |
| | G71_, C80T; TG3AC, GC7AG, GA28CT, CA32GG; T3A, G5C, G7T, C31G, A33T; |
| | T3G, G7T, C9T, C17G; G64T, A85T; G7C, T14_; C9T, A29T; G7T, \hat{ }G14; A88G, \hat{ }C89; CT2_, A33T; |
| | C81T, \hat{ }A82; C9T, C17G, A29C, C32A; C9T, C17G, \hat{ }GA77 |
| 2.5 to 2.6 | G7C, C15G; C9T, C17G, TC75GT; TG3CA, CG6GA, AG29TC, CA32GG; G7T; T14A, T16G; |
| | G7T, C9T, G71_, \hat{ }T79; C15A; CT2_, A33T, C73_; C2A, C9T, C17G; CGC6TCA, GAG28TGA; |
| | C15G, A29C; C2_, T16G, A91C; \hat{ }T81, C81T; TG3AA, A29C, CA32TG; G4A, G7T, C32T; |
| | T3C, CGC6GCT, GAG28AGC, A33G; T3A, G7A, A29T, A33G; -G.4.CC, G7_, AGCC29GCGG; |
| | C65T, G86_; C9T, \hat{ }A16; A36G, \hat{ }C57; A1_, T16G; C6T, G7T; \hat{ }G14, A29T; \hat{ }AT16, A88C; |
| | C8G, A29C; \hat{ }G64, A87C; \hat{ }G70, \hat{ }T79; T16A, \hat{ }C29; |
| | TG3GA, C6G, C8T, GAG28ACC, CA32TC, G71T; G7T, A29C; T3G, GCGSAGT, GC30CT, A33C; |
| | \hat{ }C2, \hat{ }T14, A29T; C9T, C17G, A88_; C9T, T16A |
| 2.4 to 2.5 | TGG3ACA, A29C, CCA31TGT; T3_, G5A, G7C, \hat{ }G9, \hat{ }C28, A29G, C31T, A33_; C15A, A29T; |
| | G64A, \hat{ }T65; CT2_, A27G; \hat{ }A16, \hat{ }T16; G7T, C15A; G7T, C9T, C17G; C2G, A29T, T66A; |
| | TG3GA, CGC6TTA, G28T, G30A, CA32TC; A1C, G82C; A27C, A29C; C9T, C17G, \hat{ }GA71; |
| | T3C, \hat{ }T6, CC.8.T-, C17G, GAG28AGA, A33G, \hat{ }G54; \hat{ }T16, A27T, A29C; G64C, \hat{ }A87; \hat{ }C14, A29C; |
| | \hat{ }A65, \hat{ }T65; C2T, C9T, C17G; C9T, C17A; G70A, C81A; C2G, A36T; |
| | G5C, C8G, GA28CC, CC31GA; C6T, A29C; C80T, \hat{ }G81; T-.3.CA, G7_, AG29GC, CA32TG; |
| | \hat{ }C78, G78A; G7A, T14_, CT65TC; -T.3.AA, G7_, AG29GC, CA32TG; \hat{ }C29, A29T; G7A, A29T; |
| | TG3GA, GC7CA, A29G, CA32TC; \hat{ }T64, G64A; C15A, A29C; T75A, G77T; \hat{ }A3, \hat{ }T3; A27T, A29C; |
| | T14A, A29C; T74C, G77A; G7C, A29G; C9T, C17_; G5A, G7A, A29T, C31T; \hat{ }C63, \hat{ }A63; |
| | G7T, A91G |
| 2.3 to 2.4 | CT2_, G64T, T66G; G28T, A29C; T3G, G5T, GC7CG, GA28CG, C31A; |
| | TG3AC, G7C, A29G, CA32GT; C9T, C15A, C17G, A29C, \hat{ }TG55, G57A; \hat{ }C14, A29T; |
| | C9T, C17G, GC64TG; G7A, \hat{ }T29, A36C; \hat{ }T16, \hat{ }G54; TG3CA, C8A, GA28TC, CA32GG; |
| | G7T, C9T, C69G; C9T, C17G, \hat{ }A70; A72_, T79G; T3A, G5T, C8T, GA28AC, C31A, A33T; |
| | C9T, C17G, A29C; \hat{ }G54; G7A, TC14CT, C17A; C9T, C17G; \hat{ }G70, \hat{ }T79, A88C; \hat{ }A64, \hat{ }G64; |
| | T14G, A29T; C9T, T16_; \hat{ }A14, \hat{ }T14; \hat{ }AC1, GCG.5.--T, GC30_, \hat{ }GT34; A29C, A91G; C2_, T14A; |
| | C9T, \hat{ }A17; C9T, C17G, G78A; T3G, G5A, A29C, C31T, A33C; C9T, \hat{ }G17; G7T, A29G; |
| | TG3GA, C6G, C8T, GAG28ACC, CA32TC; \hat{ }T1, CG6TC, C9T, C17G; C17A; \hat{ }T17, \hat{ }A17; |
| | T3A, G5C, GC7AG, GA28CT, C31G, A33G; \hat{ }GC72, A88C; T3G, G7T, A33C; |
| | TG3CA, CG6GA, AG29TC, CA32TG; T3G, G5C, C8G, GA28CC, C31G, A33C; \hat{ }T3, C80G; |
| | C9T, C17G, T45G, \hat{ }G54; C9T, C17G, A72C, T74G; G5C, C8G, GA28AC, C31G; A29T, G56T; |
| | G7T, C63A |
| 2.2 to 2.3 | A36T, A85C, A87T; T14A, C17G; C9T, C17G, \hat{ }G54; G4C, C8G, GA28AC, C32G, A87G; \hat{ }T72; |
| | A85C, A87C; G7T, T92C; C9T, C17G, \hat{ }C63; TG3AA, C6T, AG29CA, CA32TT; |
| | C9T, C17G, A85G, A88G; G64C, \hat{ }G88; G7A, \hat{ }T29, A68C; \hat{ }A13, T14C; |
| | C9T, C17G, \hat{ }G54, A85C, A88C; -GG.4.CAT, C9_, GAGCC28CGATG; |
| | TG3AC, C6A, AG29CT, CA32GG; C9T, \hat{ }C63; C9T, A88C; A27T, A29T; C9T, C17G, \hat{ }G54, A91C; |
| | G86A, A88T; TG3CA, GC7AA, GA28TT, CA32TG, C69T; T74G, G77T; |
| | TGG3ACA, C8G, GA28CC, CCA31TGG; G7A, C17A, \hat{ }G81; G7T, A59G; \hat{ }A65, \hat{ }G86; C73T, G78T; |
| | \hat{ }C72, \hat{ }T79; A1G, C9T, C17G; \hat{ }G1, C9T, C17G; \hat{ }G72, \hat{ }C72; C2_, A29T; \hat{ }T14, A29T; \hat{ }G64, \hat{ }T87; |
| | \hat{ }A65; \hat{ }C18, \hat{ }T18; \hat{ }G64, A88C; C9A, A29C, G57T; G7C, \hat{ }G28; G77A; G7A, TC14CT, C17G; C2_; |
| | G7C, T14A, \hat{ }T86; C9T, C17G, A53G; T3G, GC7CT, GA28AG, G86T; C9T, C17G, A29C, A91G; |
| | C9T, T16_, A91C; CT2_, \hat{ }G64, C65A; C15_; T16G, C17T; G7T, G28A |
| 2.1 to 2.2 | C9T, C17G, A29T; A87C; \hat{ }CT18, A88C; C9T, C17G, \hat{ }G64; C17G; C15T; \hat{ }T16, T79C; |
| | \hat{ }A64, G64A; A1C, T3G, C9T, C17G; GA28CC, \hat{ }T65; C15A, C17A; G78C, T79G; A29C, T58G; |
| | C2_, G7A, -C.65.AA; CT2_, A29T; T3A, A33T; G4A, CGC6GTA, G28T, G30C, C32T, T67_; |
| | C9T, C17G, C65_, A91C; \hat{ }T65, A87G; A88_; G7T, C9A; C9T, C17G, C65A; |
| | TG3GC, C6T, AG29CA, C32G; G7T, T16A; G7T, G70C, C80A; G7T, T14A; |
| | TG3AA, GC7CG, GA28CG, CA32TG; \hat{ }G54, A91C; C73_, G78_; |
| | T3C, GC5TG, C8T, GA26_, G30A, \hat{ }CG34; \hat{ }CT3, A29C; C2T, T14G; G7C, A29T; C9T, TC16GG; |
| | T3G, C8T, GA28AC, A33C; \hat{ }G16, \hat{ }T16; C9T, C17G, A36C; TGG3AAC, C8G, GAG28_, A---.33.GGGT; |
| | C9T, C17G, A87G; \hat{ }T72, T79G; \hat{ }G17, C17T; CT2_, A39C, A88C; T3G, A33C; |
| | T3_, A33G; C-.2.TG, TC75CA; G7C, C9T, C17G, \hat{ }G92; C9T, C17G, G82C; C9A, A29C; |
| | C2_, C9T, C17G, A91C; C2_, A29C, A91C; CT2_, C9T, C17G; G7T, A60G; \hat{ }C71, \hat{ }T71; |
| | C2_, G77T, A91C; C2_, A29G; \hat{ }T71, C80G; T3A, G7A, A29G, A33T; C9T, A29G |
| 2.0 to 2.1 | C65T, \hat{ }A66; CT2_, C15_, T58A, A72C; C9T, C17G, C73A, C76A; C2_, A91C; C80T; |
| | T3A, G7C, C9T, C17G; \hat{ }C63, \hat{ }G88; G7T, A61T; GC62_, C65G, T67G, A72T, T79A, AAGA.84.--- |
| | C, G89C; T3G, C9T; T16A, C17A; C6T, A29T; T3C, GC5CG, C8T, GAGC28ACCG, A33G; |
| | G7A, C15T; \hat{ }T2; C15G; C9G, A29T; C15T, A29T; G7T, \hat{ }C14; \hat{ }A64, A88T; A29C, G30A; |
| | C2_, A29C, A46C; C9T, C17G, A72T, G78A; \hat{ }A87, \hat{ }T87; C9T, A59C; |
| | TG3AC, C8A, GA28TC, CA32GG; C9T, C17G, \hat{ }G64, \hat{ }G88; A29C, G71A, C80T; |
| | T3C, A29T, AC68TA; \hat{ }A17; C9T, C17G, G64T, T66C; G7A, T16G; C17T, C65G, G86C; |
| | C69T, G82C; A1T, C2A; T14A, \hat{ }C29; \hat{ }A15, C15T; G7T, T16G; T3A, GC7CA, GA28TG, A33G; |
| | \hat{ }T81; T16C, A29C; A29C, A91C; G71A, A88T; \hat{ }C65, A87G, A91C; C9T, C17G, A29T, \hat{ }A53; |
| | G71T; \hat{ }A80, \hat{ }A80; C9T, C17G, A36G; C9T, C17G, T--.54.CTG; T16A, A29T; \hat{ }G77, T79C; |
| | C9T, C17G, G64C; TG3AC, CG6GA, AG29TC, CA32GG; A36T, C37T; A29C, \hat{ }C65, A85_; |
| | C15G, A29T; \hat{ }A70, C81T; A29T, A33G; C73A, C80T; C9T, C17G, G82_; C9T; C69T, A84G; |
| | C2_, C9T, C17G, A46C |
| 1.9 to 2.0 | C2_, A29G, A91C; A68G, T83C; C9T, T14A, C17A, \hat{ }AG85; \hat{ }T66, \hat{ }G85; |
| | G62T, CT65_, C69A, G71A, C80T, G82T, A85C, AGC88_; T3_, G5T, \hat{ }A8, -A.29.TC, C31A, A33_; |
| | G7A, T14C, C17A; T3G, CG6TC, AG29GA; \hat{ }T54; \hat{ }C8, \hat{ }T8; G7T, AA87TG; A72C, C73A; |
| | C2_, C6T; \hat{ }C29; G71C, C81_; C9T, C17G, G64_, A88_; C2_, A88T; |
| | T3G, G5C, GC7TG, G28C, C31G; C9T, C15T, C17G, A36C; G7T, T34G; T14A; \hat{ }T73, \hat{ }C78; \hat{ }G64; |
| | \hat{ }G15, C15T; A36C, \hat{ }A57; A-.72.GC, \hat{ }T79; T16A, A29C, \hat{ }A58; C9T, C17G, \hat{ }T52; C2_, A85T; |
| | \hat{ }C29, A29G; G7T, T14C; C2A, \hat{ }T57; G7T, C15G, T34G; T14G, C17T; T14C, C15T; |
| | T3G, G5A, GC7TA, G28T, C31T, A33C; \hat{ }C71, \hat{ }T79; \hat{ }T14, A29C; \hat{ }A1, A36C; \hat{ }C63, \hat{ }G89; |
| | G7C, A91G; T14C, A29C; C9T, C17G, G78T, C80T; \hat{ }G69, G82C; TGG3GCA, G7T, CCA31TGC; |
| | C6T, A29C, G71C, \hat{ }G80, A91C; A13C, A29C; \hat{ }C63, A88T; G7T, T14_; C2_, GG77AA; |
| | C9T, C17G, T58A; C2_, G77T; C2_, T3_; C9T, C17G, \hat{ }AA53, A88C; G7T, C9T; G7A; |
| | CG6GC, AG29GC, C32A; C63T, TTA66GCC, GA71_, TC79_, TAA83GGC, A87C, G89_; |
| | G7C, C17G; C2_, A46C; C9G, A29T, C37T, \hat{ }A56 |
| 1.8 to 1.9 | \hat{ }G69, A72C, G82C; \hat{ }G70, T79G; G7A, C15A; \hat{ }T36, \hat{ }A57; \hat{ }G70, \hat{ }C79; |
| | TGGCG3CACAT, GCCA30TGTG; G71A; TG3AC, C8A, A29C, CA32GT; T10G, A29C; |
| | \hat{ }A65, G77A, \hat{ }G86; C9T, C17G, A88_, A91C; \hat{ }C78, \hat{ }A78; G7T, C90T; |
| | T3G, G5A, GC7TG, G28C, C31T, A33C; G7T, C9G, G86T; A29C, C31T, A33C; A29C, G70A; |
| | A-.88.GC, A91C; \hat{ }A17, A36C; T3C, GCG5TGA, AGC29TCA, A33G; |
| | T3C, CGC6GCT, GAG28AGA, A33G, A88C; C35G, \hat{ }C58; T74A, G78C; C9T, CA17GT; |
| | G7A, C17G; C9T, C17G, \hat{ }GT70; CTG2_, A29C; C2_, A68G; \hat{ }T64, \hat{ }T88; T3G, A33T; |
| | C2_, T16G, A29C; \hat{ }A1; A36T, \hat{ }G55; C9T, C17G, C63A; C9T, A18G; C2T, A36T; \hat{ }A81, \hat{ }A81; |
| | C9T, T14G, C17G; -A.72.CC, A91C; A29T, T79G; G7A, A29T, A59G; G7C, \hat{ }C78; \hat{ }AG64, A88C; |
| | CT2_, C9T, C17G, C69T; C2_, A46C, A91C; \hat{ }C89, A91C; \hat{ }C29, A68C; C2_, G64T; -C.15.GT, A27C; |
| | CT2_, T10G, A88C; T14C, A29T; C9T, C17G, C76T; A84G, A87C; G7C, C9T, T14A, C17G, T34A; |
| | G70T, C81A; T14G; \hat{ }T3, A29T; G7T, \hat{ }T29; A29T, C65A, T67G; G64C, A87G; C9T, T14A, C17G; |
| | \hat{ }T57, A87G; TGG3ATC, A29C, CCA31GAT |
| 1.7 to 1.8 | C2_, G70A; C9T, C17G, \hat{ }GA77, A88C; C9G, C17G, A29C; \hat{ }T70, \hat{ }T81; G7C, C9T, C17G; |
| | T3G, CGC6TTG, G28C, G30A, A33C; \hat{ }A16, A68T; C9T, C17G, T67C; G7T, \hat{ }C14, A33C; |
| | G7A, T14_; \hat{ }C14, \hat{ }T14; C9T, C17G, GG77TT; C2T, C80T; \hat{ }T64, A88_; \hat{ }G54, A68C; G7T, CT9AG; |
| | C9T, C17G, T79G; T79G, C80T; \hat{ }AT3, A88C; \hat{ }AG54, A88C; C2G, A33C; C2_, A88T, A91C; |
| | C9T, C17G, T58C; C2_, C73T; TGG3CCC, C8G, GA28CC, CCA31GGG; G7T, T10G; |
| | C9T, C17G, \hat{ }A80, A91C; \hat{ }T64; T14_, A29C, A91C; G7A, G28T, AAAGCGCTTA59_; G7T, G71_; |
| | \hat{ }A17, \hat{ }A17; T14_, A29T, A91C; C17G, A72G, T74C; \hat{ }T88; CT2_, A94C; A27G, A29C; |
| | A85T, A87G; C9T, C17G, \hat{ }AA79; C9T, T14A, C17G, T34A, \hat{ }G64, G86T; C9T, C17G, T45G; |
| | C2_, C9T, C17G, C65T; \hat{ }G3, G5C, C9_, GA28CG, C32A; T74G, G78T; TG3_, --C.8.GCT, |
| | G28_, \hat{ }G33; A39T, T54A; C2_, A72G; C9T, C15T, C17G; |
| | TG3CA, CG6GA, AG29GC, CA32TG; G64C, A88G; C15A, C17G; C2_, C65A; \hat{ }G64, G86A; |
| | \hat{ }C29, A36C; G64T, T66A; TG3GT, A29C, C32A; \hat{ }A64; C81G; C9T, A72T, T79C; |
| | C9T, C17G, G77T |
| 1.6 to 1.7 | A72G; \hat{ }C14, A29C, A36C; T3C, C9T, C17G; G4C, C8G, GA28AC, C32G; C2_, G71C, \hat{ }G80; C76T; |
| | C9T, T14A; C2G, C9T, C17G; G70T, C81G; C17G, \hat{ }T54; A72C; C2_, C9G, C17G; |
| | TG3GC, C8T, GA28AC, C32G; TGG3GCT, C8G, \hat{ }CC28, CC31_; C9T, C17G, A39T, A-.53.GC; |
| | \hat{ }T16; T67C, A87C; \hat{ }G81, C81T; C76G, G78C; A1C, G56A; TG3CA, GC7AG, GA28CT, CA32GG; |
| | C9T, C17G, C65G, \hat{ }A87; G86A, A88C; G7T, C9T, C17G, \hat{ }A72, G78A; \hat{ }G70, C80A; \hat{ }A17, A68C; |
| | C2_, C80G; \hat{ }C71, \hat{ }T79, A88C; C9T, C17G, \hat{ }T57; \hat{ }T2, C9T, C17G; T45G; G64C; T14_; |
| | C65T, G86A; C69T; \hat{ }C65; G64T, C65A; T3G, GC7CT, GA28AG; \hat{ }A1, \hat{ }A53; |
| | T3A, G5C, GC7AT, GA28AT, C31G, A33T; C9T, C17G, \hat{ }CA72; C9T, C17G, C73A, T79A; |
| | C2_, A53G; TGG3GTC, C8G, GA28CC, CC31GA; \hat{ }C5, G7T, C9T, C17G; G71T, C80T; C15T, T16G; |
| | G7C, C9T, C17G, C76A, G78T; G64T, T66C; \hat{ }C65, A91C; C73T; A72C, G78T; \hat{ }C63; A68G, C81T; |
| | \hat{ }GT87, A88C; C9T, C17G, \hat{ }A78; T3A, GC5AG, C8T, GAGC28ACCT, A33T; \hat{ }A1, \hat{ }T54; |
| | A29C, G56A; C2_, C80T; \hat{ }TA17, A88C; A72G, C73T; A29C, C31T, T83C; G7T, A27T; |
| | T3C, G7T, G40A, \hat{ }T54; A88C; ; G64T, A87C; T3_, \hat{ }T9, G28_, \hat{ }G32; \hat{ }GT16, A88C; -T.3.AC, |
| | G7A, C9_, GAG28TGC, CA32GG, A84C, G86T; \hat{ }T65; C76A, G77T; \hat{ }G14, A29C; |
| | G64C, A88C; A72_, T79G, A91C; \hat{ }C29, A68C, A72C; TG3AT, GC7TT, G28A, CA32AT; |
| | C9T, C17G, T--.54.CTG, A88C; G7T, A59C; CC8GT, C17G; G7C, T14C, \hat{ }T86; |
| | \hat{ }CA3, GC5_, C8G, GA26_, G30C, \hat{ }TG34 |
| 1.5 to 1.6 | T3A, \hat{ }A5, G7_, AGC29GCT, A33G; C9T, C17G, \hat{ }C73, G78C; G71A, A72G; AG27TA, A88T; |
| | G7T, A91T; \hat{ }T57, A91C; \hat{ }T2, A68C; \hat{ }T2, A36C; G7T, T10C; \hat{ }A64, A88G; |
| | TG3CA, C6T, C8T, GAG28ACA, CA32TG; \hat{ }T54, A68C, A72C; G7T, A61G; |
| | GCGC5CAAG, GAGC28CTTG; C6T, CT9TC, C17G, A29C; \hat{ }CA63, A88C; C2_, C9T, C17G, A36C; |
| | \hat{ }G64, \hat{ }G86; \hat{ }CGGCAGAT65, T67G, \hat{ }GC69, G70T, A72G, \hat{ }GCTC75, G77T, T79C, CGTAA81_; |
| | C73T, \hat{ }G74; T14G, T16A; \hat{ }AT14, A88C; G64C, A88T; C2_, A39T, \hat{ }A55; C2_, C15T; \hat{ }G70, C81T; |
| | \hat{ }A81, C81T; \hat{ }T72, A72T; C2_, C69T; T75G, T79G; A88_, A91C; \hat{ }T7, G7T; G7A, A29T, \hat{ }A77; |
| | CC8AT, C17G; C2_, T52C; G7A, C9T, TC16CG; G70A; C9T, C17G, AA87TC; \hat{ }A53, A91C; |
| | T3A, G5C, GC7CT, GA28AG, C31G, A33T; \hat{ }G70, \hat{ }C79, A88C; \hat{ }T72, \hat{ }G77; C9T, C17G, C69T; |
| | T-.3.CA, G7A, C9_, AG29GC, CA32TG; TGG.3.-AA, \hat{ }G9, \hat{ }CGC28, A29T, CCA31_; GCGC.62.-- |
| | AA, T67C, C69A, GA71AC, TC79GT, G82T, AAGA.84.---G, GC89TT; A85G, A87G; TG3_, C--.8.TCG, |
| | GAG28CGA, C32G; T66C, A85G; \hat{ }A16, G86T, A88T; TT74GG, G--.77.AAC; C2_, T79C; |
| | C9T, \hat{ }A13, C17G, \hat{ }G54; \hat{ }C63, G64T; C2_, T83C; \hat{ }C73, \hat{ }C73; -T.3.AA, |
| | G7_, A29_, A-.33.GT, G70A; \hat{ }T16, A91C; \hat{ }T64, \hat{ }G64; T79C; C9T, C17G, G77A; |
| | \hat{ }T64, \hat{ }T64; C2_, G71A; T14C, C17G; G7C, TC14CA, C17G; A85C, A88C; |
| | \hat{ }A3, GG4TC, C9_, GA28CG, CCA31AAT; --C.63.TTT, C65_, CGGA.69.T---, TCCG.79.--- |
| | A, G86C, G89A; C9T, C17G, \hat{ }C57; C15G, T16A; C9T, C17G, \hat{ }CA64; AG39TA, T52C, T54A; |
| | C2A, A87G |
| 1.4 to 1.5 | -C.15.GT, A36C; A29C, T83C; G7T, A27G; \hat{ }C29, \hat{ }C29; \hat{ }T80, \hat{ }C80; TGGC3ACAG, GA26_, -- |
| | A.33.TGG; A72G, \hat{ }T73; C9T, C17G, T66A, A85G; \hat{ }C15, \hat{ }G15; TG3_, -- |
| | C.8.GCT, GAG28CGC, C32G; \hat{ }T19; G28A, A29C; \hat{ }G70, \hat{ }G80; CT2_, A36C, A39C; |
| | C9T, C17G, \hat{ }CC79; \hat{ }G54, A68C, A72C; \hat{ }CT78, A88C; T74G, G78C; TTC74AGG, \hat{ }AT78; |
| | C9T, C17G, C76G; |
| | \hat{ }GGCAGCTCTGA64, T66C, A68C, GA71AG, \hat{ }C75, G77T, T79C, CGTAAGAA81_; \hat{ }A1, A68C; |
| | \hat{ }A4; A72G, G78C; T3G, C8T, GA28CC, A33C; G7C, -C.80.AT; C9T, C17G, A59T; G26C, C93G; |
| | G7C, T14A, \hat{ }T86, A91C; \hat{ }G64, \hat{ }T87, A88C; A1G, A29C; C9T, C17G, \hat{ }AT78; |
| | G28T, GCCA30TTTG; C2_, T75A, G78A; TG3GA, CG6AC, AG29GT, CA32TC; |
| | A36G, \hat{ }C57, A91C; \hat{ }C72, A72C; C9T, C17G, \hat{ }G82; A27T; |
| | TG3CC, CGC6TTG, G28C, G30A, CA32GG, C80G; \hat{ }A1, \hat{ }A53, A88C; A72C, C80A; G7T, C73G; |
| | \hat{ }A15, A87G; T14_, \hat{ }C29; G7A, T14_, A91C; C15T, T16A; C15T, C17G; C65_, A88_, A94C; \hat{ }A16; |
| | C9T, C17G, \hat{ }G54, A68C; -T.3.AC, G5A, C9_, GAG28CGT, CA32GG; \hat{ }T15, \hat{ }C15; |
| | C9T, T14A, C17G, T34C, \hat{ }G64, G86T; \hat{ }T71, C80G, A91C; -C.15.GT, A68C; \hat{ }G87, \hat{ }T87; |
| | C73_, G78_, A94C; C2G; G77C, T79A; G70C; A68G; \hat{ }T81, A91C; C9T, C17G, T79A; \hat{ }T72, \hat{ }T72 |
| 1.3 to 1.4 | T66A, A88C; C76G, G77T; A53G, A59C; CTG2_, G7T; A72_, \hat{ }T79; \hat{ }AA80, A88C; |
| | TGG3CAA, C8G, GA28CC, CCA31TGG; \hat{ }C78, \hat{ }T78; --G.28.TGA, T79C; \hat{ }T72, \hat{ }G77, A88C; |
| | A72G, \hat{ }C79; T3G, G5A, G7A, A29G, C31T, A33C; T14G, A21G; \hat{ }T2, A72C; G7T, T14G, \hat{ }CG64; |
| | T3G, G71A; G64A, A87G; T3C, C6T, AG29CC, A33G; T45A; G7A, C9T, T14A, C17G; |
| | TG3CT, CGC6TAT, GAG28ATA, CA32AG; C9T, C17G, \hat{ }T83; G7T, C9T, A53T; C9T, C17G, T75G; |
| | G7C, T14C, A72_; \hat{ }A65, A87G, \hat{ }C89; C9T, C17G, G70C, C81G; G7T, A59T; AG29CA, A72T, \hat{ }G77; |
| | T74C, G78A; C2A; C9T, C17G, C73T, T75G; \hat{ }G54, A72C; \hat{ }AA81, A88C; \hat{ }T54, A68C; |
| | C65A, G86A; \hat{ }A1, A72C; T3G, C9T, C17G; C2_, A33T; A87T; \hat{ }A65, \hat{ }T86; A53G; A85G, A87C; |
| | T3G, G5C, GC7TG, G28C, C31G, TC75_; -T.3.AC, G7A, C9_, GAG28TGC, CA32GG, G71T; |
| | G7C, C15A; G64A, A85G, A88_; \hat{ }A74; \hat{ }TG64, A88C; A29C, A60T; C9T, C17G, C80G; |
| | \hat{ }T64, \hat{ }A87; G7T, \hat{ }A59; G77C, G78C; A72C, \hat{ }T79; \hat{ }T73, \hat{ }C78, A88C; \hat{ }C29, A91C; \hat{ }A64, A88C; |
| | \hat{ }G54, T58A; TGGCG3CACTT, GCCA30AGTG; C9T, C17G, A21T; |
| | G4C, C8G, GA28AC, C32G, \hat{ }G82; A36C, A53G; C9T, C17G, G71T; C9T, CA17GT, T45A, G70C; |
| | \hat{ }A81; G7A, A72T; CT2_, T10G; G64T, A87G; \hat{ }G70, T79A; C2_, C9T, C17G, T52C; C2_, T45C; |
| | C9T, C17G, \hat{ }C35, A36G; G7T, T58A; \hat{ }A73, \hat{ }C73 |
| 1.2 to 1.3 | C2G, C73G; G7T, \hat{ }T14; T75C, C76T; \hat{ }A80, \hat{ }C80; A1_, A46C; C9T, C17_, A91C; |
| | C35G, \hat{ }C58, A68C; C2T, T3A; \hat{ }C29, A72C; T79G, C80A; G71A, C81_; G7T, G28T; CT2_, T45G; |
| | A29C, \hat{ }G92; C9T, C17G, T67C, A84G; T3C, \hat{ }T6, C9_, GAG28AGA, A33G; A36T; A85C, A88T; |
| | TG3GC, C6A, C8T, GAG28ACT, CA32GC; T10C, A29C; A1_, C2_; \hat{ }C65, A87T; A72T, C81T; |
| | C15A, T79A; \hat{ }GA1, G7A, C15A, C17G, A88C; \hat{ }A16, T16A; A29T, A60C; C76A, G78A; |
| | A29T, C31T; A29C, G86C; \hat{ }G70, T79G, A91C; \hat{ }T54, A72C; \hat{ }GAAC73, T74A, GG.77.C-; |
| | T14_, A29C, A46C; C9T, C17G, \hat{ }A72, \hat{ }A78; T14C, C15A; \hat{ }A17, \hat{ }G17; C9T, C17G, CG76AC; |
| | T74C, T79C; G7A, TC14AA, C17A; \hat{ }T64, \hat{ }A64; \hat{ }T81, \hat{ }A81; C2A, A36T; C9T, C17G, G82T; |
| | T74A, G77A; \hat{ }A1, A33C, A36C; G7C, TC14CT, T34A; A36T, A53G; \hat{ }A65, \hat{ }A84; A1_; G7T, \hat{ }T60; |
| | T3A, G5C, G7T, C31G, A33T, T52G, \hat{ }C54; T75G, G77T; G5C, G7A, A29T, C31T; |
| | TGGC3CCAG, C8T, GA26_, G30A, --A.33.TGG; C9T, \hat{ }C17; C2_, T14A, A91C; G77A, G78T; |
| | \hat{ }G64, G86A, A91C; T16A, C17G; C9T, C17G, T34A; A87G; A39G, -T.54.GC; A39G, -T.54.GC, |
| | A91C; \hat{ }A5, C6T, C9_, G28C, GC30CT; A72C, G77A; C2_, A91C, A94C; C2_, G7C; A84G; |
| | C73A, G78T; \hat{ }T78, \hat{ }A78; TGG3GTC, C8G, GA28AC, CCA31GAC; G7A, \hat{ }G14; C76T, G77A; |
| | C2_, G7T; G7A, T14A; \hat{ }A17, A68C, A72C; TGG3CCA, GC7CG, GA28CG, CCA31TGG; T79G; |
| | \hat{ }A72, \hat{ }C78; C15G, A29T, G57C, A59T; T14A, \hat{ }G74; G7T, C65T, A87C; C9T, C17G, G70T |

*mutated sequences are ‘;’-separated and multiple mutations per sequence are ‘,’-separated

### Example 29: The CcdB Selection Assay Identifies CasX Protein Variants with Improved dsDNA Cleavage or Improved Spacer Specificity at TTC, ATC, and CTC PAM Sequences

Experiments were conducted to identify the set of variants derived from CasX 515 (SEQ ID NO: 145) that were biochemically competent and that exhibit improved activity or improved spacer specificity compared to CasX 515 for double-stranded DNA (dsDNA) cleavage at target DNA sequences associated with a PAM sequence of either TTC or ATC or CTC. In order to accomplish this, first, a set of spacers was identified with survival above background levels in a CcdB selection experiment using CasX 515 and guide scaffold 174. Second, CcdB selections were performed with these spacers to determine the set of variants derived from CasX 515 that are biochemically competent for dsDNA cleavage at the canonical “wild-type” PAM sequence TTC. Third, CcdB selection experiments were performed to determine the set of variants of CasX 515 that enable improved dsDNA cleavage at either PAM sequences of type ATC or of type CTC. Fourth, plasmid counter-selection experiments were performed to determine the set of variants derived from CasX 515 that resulted in improved spacer specificity.

### Materials and Methods

For CcdB selection experiments, 300 ng of plasmid DNA (p73) expressing the indicated CasX protein (or library) and sgRNA was electroporated into E. coli strain BW25113 harboring a plasmid expressing the CcdB toxic protein. Cells were titered on plates containing either glucose (CcdB toxin is not expressed) or arabinose (CcdB toxin is expressed), and the relative survival was calculated and plotted, as shown in FIG. 103. The remainder of the recovered culture was split after the recovery period, and grown in media containing either glucose or arabinose, in order to collect samples of the pooled library either with no selection, or with strong selection, respectively. These cultures were harvested and the surviving plasmid pool was extracted using a Plasmid Miniprep Kit (QIAGEN®) according to the manufacturer's instructions. The entire process was repeated for a total of three rounds of selection.

The final plasmid pool was isolated and a PCR amplification of the p73 plasmid was performed using primers specific for unique molecular identifier (UMI). These UMI sequences had been designed such that each specific UMI is associated with one and only one single mutation of the CasX 515 protein. Typical PCR conditions were used for the amplification. The pool of variants of the CasX 515 contained many possible amino acid substitutions, as well as possible insertions, and single amino acid deletions in an approach termed Deep Mutational Evolution (DME). Amplified DNA product was purified with Ampure XP DNA cleanup kit. Amplicons were then prepared for sequencing with a second PCR to add adapter sequences compatible with next-generation sequencing (NGS) on either a MiSeq™ instrument or a NextSeq instrument (Illumina™) according to the manufacturer's instructions. NGS of the prepared samples was performed. Returned raw data files were processed as follows: (1) the sequences were trimmed for quality and for adapter sequences; (2) the sequences from read 1 and read 2 were merged into a single insert sequence; and (3) each sequence was quantified for containing a UMI associated with a mutation relative to the reference sequence for CasX 515. Incidences of individual mutations relative to CasX 515 were counted. Mutation counts post-selection were divided by mutation counts pre-selection, and a pseudocount of ten was used to generate an “enrichment score”. The log base two (log₂) of this score was calculated and plotted as heat maps in which the enrichment score for biological replicates for a single spacer was determined at each amino acid position for insertions, deletions, or substitutions (not shown). The library was passed through the CcdB selection with two TTC PAM spacers performed in triplicate (spacers 23.2 AGAGCGTGATATTACCCTGT, SEQ ID NO: 2924, and 23.13 CCCTTTGACGTTGGAGTCCA, SEQ ID NO: 2925) and one TTC PAM spacer performed in duplicate (spacer 23.11 TCCCCGATATGCACCACCGG, SEQ ID NO: 2926), and the mean of triplicate measurements was plotted on a log 2 enrichment scale as a heatmap for the measured variants of CasX 515. Variants of CasX 515 that retained full cleavage competence compared to CasX 515 exhibited log 2 enrichment values around zero; variants with loss of cleavage function exhibited log 2 values less than zero, while variants with improved cleavage using this selection resulted in log 2 values greater than zero compared to the values of CasX 515. Experiments to generate additional heat maps (not shown) were performed using the following single spacers (11.2 AAGTGGCTGCGTACCACACC, SEQ ID NO: 2927; 23.27 GTACATCCACAAACAGACGA, SEQ ID NO: 2928; and 23.19 CCGATATGCACCACCGGGTA, SEQ ID NO: 2929, respectively) for selectivity.

For plasmid counter-selection experiments, additional rounds of bacterial selection were performed on the final plasmid pool that resulted from CcdB selection with TTC PAM spacers. The overall scheme of the counter-selection is to allow replication of only those cells of E. coli which contain two populations of plasmids simultaneously. The first plasmid (p73) expresses a CasX protein (under inducible expression by ATc) and a sgRNA (constitutively expressed), as well as an antibiotic resistance gene (chloramphenicol). Note that this plasmid can also be used for standard forward selection assays, such as CcdB, and that the spacer sequence is completely free to vary as desired by the experimentalist. The second plasmid (p74) serves only to express an antibiotic resistance gene (kanamycin) but has been modified to contain (or not contain) target sites matching the spacer encoded in p73. Furthermore, these target sites can be designed to incorporate “mismatches” relative to the spacer sequence, consisting of non-canonical Watson-Crick base-pairing between the RNA of the spacer and the DNA of the target site. If the RNP expressed from p73 is able to cleave a target site in p74, the cell will remain only resistant to chloramphenicol. In contrast, if the RNP cannot cleave the target site, the cell will remain resistant to both chloramphenicol and kanamycin. Finally, the dual plasmid replication system described above can be achieved in two ways. In sequential methods, either plasmid can be delivered to a cell first, after which the strain is made electrocompetent and the second plasmid is delivered (both by electroporation). Previous work has shown that either order of plasmid delivery is sufficient for successful counter-selection, and both schemes were performed: in an experiment named “Screen 5”, p73 is electroporated into competent cells harboring p74, while in Screen 6 the inverse is true. Cultures were electroporated, recovered, titered, and grown under selective conditions as above for a single round, and plasmid recovery followed by amplification, NGS, and enrichment calculation were also performed as above.

Finally, additional CcdB selections were performed in a similar manner, but with guide scaffold 235 and with alternative promoters WGAN45, Ran2, and Ran4, all targeting the toxic CcdB plasmid with spacer 23.2. These promoters are expected to more weakly express the guide RNA compared to the above CcdB selections and are thus expected to reduce the total concentration of CasX RNP in a bacterial cell. This physiological effect should reduce the overall survival of bacterial cells in the selective assay, thus increasing the dynamic range of enrichment scores and correlating more precisely with RNP nuclease activity at the TTC PAM spacer 23.2. For each promoter, three rounds of selection were performed in triplicate as above, and each round of experimentation resulted in enrichment data as above. These experiments are hereafter referred to as Screen 7.

### Results:

The results of the library screen heat maps demonstrated that CasX 515 complexed with guide scaffold 174 was capable of cleaving the CcdB expression plasmid when targeted using spacers (listed below) that target DNA sequences associated with TTC PAM sequences. In contrast, spacers utilizing alternative PAM sequences exhibited far more variable survival. ATC PAM spacers (listed below) ranged in survival from a few percent to much less than 0.1%, while CTC PAM spacers (listed below) enabled survival in a range from >50% to less than 1%. Finally, GTC PAM spacers (listed below) only enabled survival at or below 0.1%. These benchmarking data support the experimental design of this selection pipeline and demonstrate the robust selective power of the CcdB bacterial assay. Specifically, CasX proteins unable to cleave double-stranded DNA are de-enriched by at least four orders of magnitude, while CasX proteins biochemically competent for cleavage will survive the assay.

Heatmaps were used to identify the set of variants of CasX 515 that were biochemically competent for dsDNA cleavage at target DNA sequences associated with a TTC PAM sequence, as well as those variants exhibiting improved for dsDNA cleavage at target DNA sequences associated with PAM sequences of CTC (spacers 11.2 and 23.27) and ATC (spacer (23.19).

These three datasets, either individually, or combined, represent underlying biochemical differences between variants and identify regions of interest for future engineering of improved CasX therapeutics for human genome editing. As evidence for this, internal controls were included uniformly as part of the na Ove library, such as the presence of a stop codon at each position throughout the protein. These stop codons were consistently observed to be lost throughout rounds of selection, consistent with the expectation that partially truncated CasX 515 should not enable dsDNA cleavage. Similarly, variants with a loss of activity reflected in the heatmap data were observed to have become depleted during the selection, and thus have a severe loss of fitness for double-stranded DNA cleavage in this assay. However, variants with an enrichment value of one or greater (and a corresponding log 2 enrichment value of zero or greater) are, at minimum, neutral with respect to biochemical cleavage. Importantly, if one or more of the mutations identified in this specific subset of variants exhibit desirable properties for a therapeutic molecule, these mutations establish a structure-function relationship shown to be compatible with biochemical function. More specifically, these mutations can affect properties such as CasX protein transcription, translation, folding, stability, ribonucleoprotein (RNP) formation, PAM recognition, double-stranded DNA unwinding, non-target strand cleavage, and target strand cleavage.

For those variants competent for cleavage at sequences associated with CTC and ATC PAM sequences, enriched variants in these datasets (enrichment >1, equivalent to log 2 enrichment for values of approximately 0) represent mutations that specifically improve cleavage of CTC or ATC PAM target sites. Mutations meeting these criteria can be further subcategorized in two general ways: either the mutation improves cleavage rates by improving the recognition of the PAM (Type 1) or the mutation improves the overall cleavage rate of the molecule regardless of the PAM sequence (Type 2).

As examples of the first type, substitution mutations at position 223 were found to be enriched by several hundred-fold in all samples tested. This location encodes a glycine in both wild-type reference CasX proteins CasX 1 and 2, which is measured to be 6.34 angstroms from the ˜4 nucleotide position of the DNA non-target strand in the published CryoEM structure of CasX 1 (PDB ID: 6NY2). These substitution mutations at position 223 are thus physically proximal to the altered nucleotide of the novel PAM, and likely interact directly with the DNA. Further supporting this conclusion, many of the enriched substitutions encoded amino acids which are capable of forming additional hydrogen bonds relative to the replaced amino acid (glycine). These findings demonstrate that improved recognition of novel PAM sequences can be achieved in the CasX protein by introducing mutations that interact with one or both of the DNA strands, especially when physically proximal to the PAM DNA sequence (within ten angstroms). Additional features of the heat maps for ATC and CTC spacers represented mutations enabling increased recognition of non-canonical PAM sequences, but their mechanism of action has not yet been investigated.

As examples for the second type of mutation, the results of the heat maps were used to identify mutations that improve the overall cleavage rate compared to CasX 515, but without necessarily specifically recognizing the PAM sequence of the DNA. For example, a variant of CasX 515 consisting of an insertion of arginine at position 27 was measured to have an enrichment value greater than one in the selection with spacer 11.2 (CTC PAM) and spacer 23.19 (ATC PAM). This variant had previously been identified by a comparable selection on a CTC PAM spacer, where this mutation was enriched by orders of magnitude (data not shown). The position of this amino acid mutation is physically proximal (9.29 angstroms) to the DNA target strand at position −1 in the above structural model. These insights suggest a mechanism where the mature R-loop formed by CasX RNP with double-stranded DNA is stabilized by the side chain of the arginine, perhaps by ionic interactions of the positively charged side chain with the negatively charged backbone of the DNA target strand. Such an interaction is beneficial to overall cleavage kinetics without altering the PAM specificity. These data support the conclusion that some enriched mutations represent variants that improve the overall cleavage activity of CasX 515 by physically interacting with either or both of the DNA strands when physically proximal to them (within ten angstroms).

The data support the conclusion that many of the mutations measured to improve cleavage at sequences associated with the CTC or ATC PAM sequences identified from the heat maps can be classified as either of the two types of mutations specified above. For mutations of type one, variants consisting of mutations to position 223 with a large enrichment score in at least one of the spacers tested at CTC PAMs are listed in Table 49, with the associated maximum enrichment score. For mutations of type two, a smaller list of mutations was chosen systematically from among the thousands of enriched variants. To identify those mutations highly likely to improve the overall cleavage activity compared to CasX 515, the following approach was taken. First, mutations were filtered for those which were most consistently enriched across CTC or ATM PAM spacers. A lower bound (LB) was defined for the enrichment score of each mutation for each spacer. LB was defined as the combined log 2 enrichment score across biological triplicates, minus the standard deviation of the log 2 enrichment scores for the individual replicates. Second, the subset of these mutations was taken in which LB>1 for at least two out of three independent experimental datasets (one ATC PAM selection and two CTC PAM selections). Third, this subset of mutations was further reduced by excluding those for which a negative log 2 enrichment was measured in any of the three TTC PAM selections. Finally, individual mutations were manually selected based on a combination of structural features and strong enrichment score in at least one experiment. The resulting 274 mutations meeting these criteria are listed in Table 50, along with the maximum observed log 2 enrichment score from the two CTC or one ATC PAM experiments represented in the resulting heat maps, as well as the domain in which the mutation is located.

In contrast to Class I mutations, there exists another category of mutations that improve the ability of the CasX RNP to discriminate between on-target and off-target sites in genomic DNA, as determined by the spacer sequence, termed Class II, which improve the spacer specificity of the nuclease activity of the CasX protein. Two additional experiments were performed to specifically identify Class II mutations, where these experiments consisted of plasmid counter-selections and resulted in enrichment scores representing the sensitivity of the generated variant, compared to CasX 515, to a single mismatch between the spacer sequence of the guide RNA and the intended target DNA. The resulting enrichment scores were ranked for all observed mutations across the experimental data, and the following analyses were performed to identify a subset of mutations likely to improve the spacer specificity of the CasX protein without substantially reducing the nuclease activity at the desired on-target site. First, mutations from Screen 5 were ranked by their average enrichment score across three technical replicates using Spacer 23.2. Those mutations which were physically proximal to the nucleotide mismatch, as inferred from published models of the CasX RNP bound to a target site (PDB ID: 6NY2), were removed in order to discard those Class II mutations that might only confer improvements to specificity at Spacer 23.2 only, rather than universally across spacers. Finally, these Class II mutations were discarded if their cleavage activity at on-target TTC PAM sites was negatively impacted by the mutation if their average log 2 enrichment from the three TTC PAM CcdB selections was less than zero. The resulting mutations meeting these criteria are listed in Table 51, along with the maximum observed log₂ enrichment score from Screen 5 and the domain in which the mutation is located. Additionally, Class II mutations were identified from the counter-selection experiment Screen 6. These mutations were similarly ranked by their mean enrichment scores, but different filtering steps were applied. In particular, mutations were identified from each of the following categories: those with the highest mean enrichment scores from either Spacer 23.2, Spacer 23.11, or Spacer 23.13; those with the highest combined mean enrichment scores from Spacer 23.2 and Spacer 23.11; those with the highest combined mean enrichment scores from Spacer 23.11 and Spacer 23.13; or those with the highest combined mean enrichment scores from Spacer 23.2 in Screen 5 and Spacer 23.2 in Screen 6. These resulting mutations are listed in Table 51, along with the maximum observed log 2 enrichment score from Screen 6 and the domain in which the mutation is located.

In addition to the Class I or Class II mutations, there exists another category of mutations that has been directly observed to improve the dsDNA editing activity at TTC PAM sequences. These mutations, termed Class III mutations, demonstrated improved nuclease activity by way of exhibiting enrichment scores above that of CasX 515 when targeting the CcdB plasmid using Spacer 23.2 in Screen 7. A computational filtering step was used to identify a subset of these enriched mutations which are of particular interest. Specifically, mutations were identified that had an average enrichment value across three replicates that was greater than zero for each of the three promoters tested. Finally, features of the enrichment scores across the amino acid sequence were used to identify additional mutations at enriched positions. Example features of interest included the following: insertions or deletions at the junction of protein domains in order to facilitate topological changes; substitutions of an amino acid for proline in order to kink the polypeptide backbone; substitutions of an amino acid for a positively charged amino acid in order to add ionic bonding between the protein and the negatively charged nucleic acid backbone of either the guide RNA or either strand of the target DNA; deletions of an amino acid where consecutive deletions are both highly enriched; substitutions to a position that contains many highly enriched substitutions; substitutions of an amino acid for a highly enriched amino acid at the extreme N-terminus of the protein. These resulting mutations are listed in Table 52, along with the maximum observed log 2 enrichment score from Screen 6 and the domain in which the mutation is located.

TABLE 49
Mutations to CasX 515 (SEQ ID NO: 145) that improve
cleavage activity at CTC PAM sequences by physically
interacting with the PAM nucleotides of the DNA

| | | | Maximum observed | |
|---|---|---|---|---|
| | | | log₂ enrichment | |
| Position | Reference | Alternate | in Ccdb selections | Domain |
| 223 | G | Y | 4.6 | helical I-II |
| 223 | G | N | 5.7 | helical I-II |
| 223 | G | H | 4.2 | helical I-II |
| 223 | G | S | 4.6 | helical I-II |
| 223 | G | T | 3.8 | helical I-II |
| 223 | G | A | 6.3 | helical I-II |
| 223 | G | V | 3.6 | helical I-II |

TABLE 50
Mutations to CasX 515 systematically identified from all datasets
to improve cleavage activity at ATC and CTC PAM sequences

| | | | Maximum observed | |
|---|---|---|---|---|
| | | | log₂ enrichment | |
| Position | Reference | Alternate | in CcdB selections | Domain |
| 3 | — | G | 3.0 | OBD-I |
| 3 | I | G | 3.5 | OBD-I |
| 3 | I | E | 4.5 | OBD-I |
| 4 | — | G | 2.5 | OBD-I |
| 4 | K | G | 2.5 | OBD-I |
| 4 | K | P | 3.1 | OBD-I |
| 4 | K | S | 3.3 | OBD-I |
| 4 | K | W | 2.8 | OBD-I |
| 5 | — | P | 3.5 | OBD-I |
| 5 | — | G | 3.1 | OBD-I |
| 5 | R | S | 3.7 | OBD-I |
| 5 | — | S | 2.2 | OBD-I |
| 5 | R | A | 3.2 | OBD-I |
| 5 | R | P | 3.6 | OBD-I |
| 5 | R | G | 3.2 | OBD-I |
| 5 | R | L | 2.7 | OBD-I |
| 6 | I | A | 3.3 | OBD-I |
| 6 | — | G | 3.7 | OBD-I |
| 7 | N | Q | 3.1 | OBD-I |
| 7 | N | L | 2.7 | OBD-I |
| 7 | N | S | 3.7 | OBD-I |
| 8 | K | G | 3.3 | OBD-I |
| 15 | K | F | 3.0 | OBD-I |
| 16 | D | W | 2.8 | OBD-I |
| 16 | — | F | 4.2 | OBD-I |
| 18 | — | F | 3.5 | OBD-I |
| 28 | M | H | 2.5 | OBD-I |
| 33 | V | T | 2.0 | OBD-I |
| 34 | R | P | 3.6 | OBD-I |
| 36 | M | Y | 2.4 | OBD-I |
| 41 | R | P | 2.2 | OBD-I |
| 47 | L | P | 2.2 | OBD-I |
| 52 | E | P | 3.2 | OBD-I |
| 55 | — | P | 2.7 | OBD-I |
| 55 | PQ | — | 3.0 | OBD-I |
| 56 | Q | S | 1.9 | OBD-I |
| 56 | — | D | 2.5 | OBD-I |
| 56 | — | T | 2.8 | OBD-I |
| 56 | Q | P | 3.9 | OBD-I |
| 58 | — | A | 2.2 | helical I-I |
| 63 | R | S | 3.0 | helical I-I |
| 63 | R | Q | 2.7 | helical I-I |
| 72 | D | E | 2.7 | helical I-I |
| 81 | L | V | 2.8 | helical I-I |
| 81 | L | T | 2.7 | helical I-I |
| 85 | W | G | 3.2 | helical I-I |
| 85 | W | F | 2.7 | helical I-I |
| 85 | W | E | 2.9 | helical I-I |
| 85 | W | D | 3.1 | helical I-I |
| 85 | W | A | 2.8 | helical I-I |
| 85 | W | Q | 3.0 | helical I-I |
| 85 | W | R | 3.7 | helical I-I |
| 88 | F | M | 2.4 | helical I-I |
| 89 | Q | D | 2.5 | helical I-I |
| 93 | V | L | 1.9 | helical I-I |
| 109 | Q | P | 1.8 | NTSB |
| 115 | E | S | 1.8 | NTSB |
| 120 | G | D | 2.4 | NTSB |
| 133 | G | T | 2.2 | NTSB |
| 141 | L | A | 2.2 | NTSB |
| 168 | L | K | 3.1 | NTSB |
| 170 | A | Y | 2.7 | NTSB |
| 170 | A | S | 1.7 | NTSB |
| 175 | E | A | 2.0 | NTSB |
| 175 | E | D | 2.8 | NTSB |
| 175 | E | P | 3.8 | NTSB |
| 223 | G | — | 1.4 | helical I-II |
| 223 | G | S | 8.8 | helical I-II |
| 223 | G | T | 3.7 | helical I-II |
| 242 | S | T | 1.9 | helical I-II |
| 247 | I | T | 1.8 | helical I-II |
| 254 | V | T | 2.5 | helical I-II |
| 265 | L | Y | 1.9 | helical I-II |
| 288 | K | G | 4.2 | helical I-II |
| 288 | K | S | 4.0 | helical I-II |
| 291 | V | L | 2.6 | helical I-II |
| 303 | M | T | 2.3 | helical I-II |
| 303 | M | W | 2.7 | helical I-II |
| 328 | G | N | 3.3 | helical I-II |
| 331 | S | Q | 2.7 | helical I-II |
| 334 | — | A | 2.3 | helical II |
| 334 | LV | — | 3.0 | helical II |
| 335 | V | E | 2.8 | helical II |
| 335 | V | Q | 2.7 | helical II |
| 335 | V | F | 2.5 | helical II |
| 335 | V | — | 3.2 | helical II |
| 336 | E | P | 2.9 | helical II |
| 336 | E | — | 3.1 | helical II |
| 336 | E | D | 2.7 | helical II |
| 336 | E | L | 2.4 | helical II |
| 336 | E | R | 2.7 | helical II |
| 337 | R | N | 2.5 | helical II |
| 338 | Q | V | 2.5 | helical II |
| 338 | — | Q | 3.0 | helical II |
| 339 | — | G | 2.6 | helical II |
| 341 | — | H | 2.9 | helical II |
| 341 | — | A | 2.0 | helical II |
| 342 | V | D | 2.7 | helical II |
| 342 | — | T | 2.3 | helical II |
| 342 | V | — | 3.0 | helical II |
| 342 | — | F | 2.5 | helical II |
| 343 | — | D | 3.3 | helical II |
| 343 | D | — | 2.0 | helical II |
| 344 | W | — | 3.1 | helical II |
| 344 | W | T | 2.8 | helical II |
| 344 | W | H | 2.8 | helical II |
| 344 | — | P | 3.0 | helical II |
| 344 | — | G | 2.6 | helical II |
| 345 | — | R | 3.2 | helical II |
| 345 | W | P | 3.1 | helical II |
| 345 | W | D | 2.3 | helical II |
| 345 | — | D | 2.9 | helical II |
| 345 | W | L | 2.3 | helical II |
| 346 | — | P | 2.4 | helical II |
| 346 | — | D | 2.9 | helical II |
| 347 | M | — | 2.6 | helical II |
| 348 | — | T | 3.3 | helical II |
| 350 | N | I | 2.3 | helical II |
| 351 | V | N | 2.8 | helical II |
| 351 | V | H | 3.1 | helical II |
| 352 | K | D | 2.2 | helical II |
| 354 | L | D | 3.1 | helical II |
| 355 | I | S | 2.6 | helical II |
| 357 | E | C | 2.1 | helical II |
| 357 | E | P | 2.8 | helical II |
| 358 | K | T | 2.8 | helical II |
| 359 | K | E | 2.7 | helical II |
| 363 | K | L | 3.3 | helical II |
| 363 | K | Y | 2.2 | helical II |
| 367 | Q | D | 2.8 | helical II |
| 367 | Q | P | 3.0 | helical II |
| 369 | — | S | 2.6 | helical II |
| 369 | LA | — | 2.4 | helical II |
| 373 | K | L | 2.2 | helical II |
| 374 | — | R | 2.0 | helical II |
| 397 | Y | T | 2.5 | helical II |
| 400 | G | M | 2.0 | helical II |
| 402 | L | V | 2.4 | helical II |
| 403 | L | C | 2.3 | helical II |
| 404 | L | D | 2.5 | helical II |
| 404 | L | N | 2.5 | helical II |
| 404 | L | W | 2.3 | helical II |
| 404 | L | Y | 2.1 | helical II |
| 407 | E | F | 2.6 | helical II |
| 407 | E | L | 2.2 | helical II |
| 407 | E | Y | 2.6 | helical II |
| 411 | G | P | 2.6 | helical II |
| 411 | — | E | 3.2 | helical II |
| 413 | — | T | 2.7 | helical II |
| 413 | — | R | 2.4 | helical II |
| 413 | — | W | 3.0 | helical II |
| 413 | — | Y | 3.7 | helical II |
| 414 | — | W | 2.6 | helical II |
| 414 | — | Y | 3.1 | helical II |
| 414 | W | G | 3.0 | helical II |
| 414 | W | R | 2.6 | helical II |
| 416 | K | D | 2.7 | helical II |
| 416 | K | H | 2.0 | helical II |
| 416 | K | P | 2.6 | helical II |
| 416 | K | T | 2.3 | helical II |
| 417 | V | L | 2.6 | helical II |
| 417 | V | A | 2.5 | helical II |
| 418 | Y | C | 2.7 | helical II |
| 419 | D | G | 3.2 | helical II |
| 419 | D | M | 2.4 | helical II |
| 419 | D | P | 2.4 | helical II |
| 425 | I | C | 2.2 | helical II |
| 427 | K | T | 2.4 | helical II |
| 428 | K | R | 2.5 | helical II |
| 430 | E | G | 1.9 | helical II |
| 432 | L | A | 1.9 | helical II |
| 434 | K | H | 2.2 | helical II |
| 436 | I | T | 2.4 | helical II |
| 436 | I | S | 3.0 | helical II |
| 436 | I | Q | 2.7 | helical II |
| 437 | K | D | 3.1 | helical II |
| 442 | R | D | 2.5 | helical II |
| 442 | R | — | 2.7 | helical II |
| 446 | D | E | 2.3 | helical II |
| 446 | — | D | 2.3 | helical II |
| 450 | K | P | 2.3 | helical II |
| 452 | A | R | 2.0 | helical II |
| 453 | L | T | 3.2 | helical II |
| 456 | W | L | 2.2 | helical II |
| 457 | L | C | 2.2 | helical II |
| 459 | A | L | 2.0 | helical II |
| 461 | A | T | 2.7 | helical II |
| 461 | A | K | 2.1 | helical II |
| 465 | I | E | 3.1 | helical II |
| 465 | — | C | 2.9 | helical II |
| 466 | — | S | 3.5 | helical II |
| 466 | — | G | 2.5 | helical II |
| 467 | — | R | 2.4 | helical II |
| 467 | G | P | 2.0 | helical II |
| 468 | L | K | 3.6 | helical II |
| 468 | L | D | 3.2 | helical II |
| 468 | L | S | 3.0 | helical II |
| 468 | L | H | 3.3 | helical II |
| 470 | E | — | 2.4 | helical II |
| 472 | D | R | 2.2 | helical II |
| 472 | — | D | 2.4 | helical II |
| 473 | — | P | 2.6 | helical II |
| 474 | — | D | 2.7 | helical II |
| 475 | EF | — | 2.8 | helical II |
| 475 | — | Q | 2.7 | helical II |
| 476 | F | K | 2.8 | helical II |
| 476 | F | — | 2.2 | helical II |
| 477 | — | G | 2.8 | helical II |
| 479 | C | D | 3.1 | helical II |
| 480 | — | V | 2.2 | helical II |
| 480 | E | D | 2.3 | helical II |
| 481 | — | H | 2.2 | helical II |
| 481 | L | R | 2.9 | helical II |
| 482 | K | R | 2.1 | helical II |
| 483 | L | H | 2.7 | helical II |
| 484 | Q | C | 2.1 | helical II |
| 485 | K | P | 3.0 | helical II |
| 490 | L | S | 2.8 | helical II |
| 498 | E | L | 2.1 | helical II |
| 499 | — | F | 1.6 | helical II |
| 511 | K | T | 6.8 | OBD-II |
| 524 | — | P | 2.4 | OBD-II |
| 553 | — | S | 2.4 | OBD-II |
| 558 | — | R | 1.9 | OBD-II |
| 570 | M | T | 2.7 | OBD-II |
| 582 | I | T | 1.9 | OBD-II |
| 592 | Q | I | 2.1 | OBD-II |
| 592 | Q | F | 2.8 | OBD-II |
| 592 | Q | V | 2.0 | OBD-II |
| 592 | Q | A | 2.9 | OBD-II |
| 641 | — | R | 2.3 | OBD-II |
| 643 | — | D | 2.7 | OBD-II |
| 644 | — | W | 2.5 | OBD-II |
| 645 | — | A | 2.4 | OBD-II |
| 650 | — | I | 2.5 | RuvC-I |
| 651 | — | S | 2.4 | RuvC-I |
| 652 | — | T | 2.4 | RuvC-I |
| 652 | — | N | 2.3 | RuvC-I |
| 653 | — | R | 2.3 | RuvC-I |
| 653 | — | K | 2.2 | RuvC-I |
| 654 | — | H | 2.2 | RuvC-I |
| 654 | — | S | 2.3 | RuvC-I |
| 658 | V | L | 1.9 | RuvC-I |
| 695 | G | W | 1.4 | RuvC-I |
| 695 | G | R | 3.5 | RuvC-I |
| 708 | K | S | 3.0 | RuvC-I |
| 708 | K | T | 2.9 | RuvC-I |
| 708 | K | E | 3.1 | RuvC-I |
| 711 | V | A | 1.6 | RuvC-I |
| 726 | K | E | 2.0 | RuvC-I |
| 729 | N | G | 2.8 | RuvC-I |
| 736 | R | H | 2.7 | RuvC-I |
| 736 | R | G | 2.4 | RuvC-I |
| 771 | M | S | 3.7 | RuvC-I |
| 771 | M | A | 3.3 | RuvC-I |
| 792 | L | F | 2.5 | RuvC-I |
| 868 | V | D | 1.9 | TSL |
| 877 | — | A | 2.0 | TSL |
| 886 | T | E | 1.8 | TSL |
| 886 | T | D | 2.5 | TSL |
| 886 | T | N | 1.6 | TSL |
| 888 | G | D | 2.5 | TSL |
| 890 | S | — | 3.0 | TSL |
| 891 | G | — | 2.7 | TSL |
| 892 | — | E | 2.0 | TSL |
| 892 | — | N | 2.9 | TSL |
| 895 | S | I | 1.7 | TSL |
| 908 | E | D | 1.7 | TSL |
| 932 | S | M | 2.5 | RuvC-II |
| 932 | S | V | 2.6 | RuvC-II |
| 944 | — | L | 1.4 | RuvC-II |
| 947 | — | G | 1.9 | RuvC-II |
| 949 | T | — | 1.9 | RuvC-II |
| 951 | G | I | 3.7 | RuvC-II |

TABLE 51
Mutations to CasX 515 systematically identified
from all datasets to improve spacer specificity

| | | | Maximum observed | |
|---|---|---|---|---|
| | | | log₂ enrichment | |
| Position | Reference | Alternate | in counter-selections | Domain |
| 6 | I | L | 2.25 | OBD-I |
| 48 | — | P | 2 | OBD-I |
| 87 | — | G | 3.96 | helical I-I |
| 90 | K | V | 4.84 | helical I-I |
| 155 | F | V | 2.13 | NTSB |
| 215 | — | T | 2.03 | helical I-II |
| 216 | — | C | 3.03 | helical I-II |
| 220 | Y | F | 2.1 | helical I-II |
| 264 | S | H | 3.16 | helical I-II |
| 329 | — | Q | 2.71 | helical I-II |
| 343 | D | S | 2.69 | helical II |
| 346 | DM | — | 2.96 | helical II |
| 349 | — | P | 2.06 | helical II |
| 357 | — | G | 2.11 | helical II |
| 375 | QE | — | 2.34 | helical II |
| 378 | L | N | 2.38 | helical II |
| 389 | K | Q | 2.29 | helical II |
| 417 | — | L | 2.75 | helical II |
| 441 | E | L | 2.36 | helical II |
| 458 | R | D | 2.2 | helical II |
| 459 | A | E | 2.65 | helical II |
| 476 | FC | — | 2.34 | helical II |
| 503 | IL | — | 2.15 | OBD-II |
| 537 | K | G | 2.85 | OBD-II |
| 621 | L | T | 2.45 | OBD-II |
| 624 | — | A | 3 | OBD-II |
| 783 | L | Y | 2.08 | RuvC-I |
| 783 | — | P | 2.6 | RuvC-I |
| 787 | L | — | 2.49 | RuvC-I |
| 787 | L | R | 3.58 | RuvC-I |
| 787 | L | D | 5.58 | RuvC-I |
| 788 | — | Q | 2.65 | RuvC-I |
| 789 | — | R | 2.5 | RuvC-I |
| 789 | — | N | 2.71 | RuvC-I |
| 790 | E | N | 2.45 | RuvC-I |
| 792 | — | P | 2.85 | RuvC-I |
| 793 | P | A | 2.93 | RuvC-I |
| 795 | K | Q | 2.45 | RuvC-I |
| 796 | T | V | 2.75 | RuvC-I |
| 798 | — | R | 4.07 | RuvC-I |
| 799 | — | H | 2.79 | RuvC-I |
| 801 | T | Q | 3.16 | RuvC-I |
| 801 | — | H | 3.34 | RuvC-I |
| 801 | — | R | 2.86 | RuvC-I |
| 802 | — | L | 2.88 | RuvC-I |
| 802 | L | — | 2.87 | RuvC-I |
| 802 | — | W | 3.08 | RuvC-I |
| 803 | — | A | 3.19 | RuvC-I |
| 803 | — | F | 3.14 | RuvC-I |
| 803 | A | S | 5.79 | RuvC-I |
| 804 | Q | K | 3.05 | RuvC-I |
| 805 | Y | — | 3.29 | RuvC-I |
| 806 | T | Y | 3.07 | RuvC-I |
| 806 | T | F | 2.49 | RuvC-I |
| 807 | — | I | 3.21 | RuvC-I |
| 807 | S | P | 2.61 | RuvC-I |
| 809 | T | P | 3.2 | RuvC-I |
| 809 | — | N | 3.1 | RuvC-I |
| 810 | C | K | 3.19 | RuvC-I |
| 810 | C | M | 3.08 | RuvC-I |
| 811 | — | M | 2.51 | TSL |
| 812 | N | — | 3.07 | TSL |
| 812 | — | V | 2.68 | TSL |
| 813 | C | S | 2.3 | TSL |
| 814 | — | G | 3.15 | TSL |
| 814 | — | W | 3.04 | TSL |
| 815 | F | P | 3.09 | TSL |
| 817 | — | W | 2.87 | TSL |
| 828 | K | G | 1.99 | TSL |
| 906 | V | C | 2.01 | TSL |

TABLE 52
Mutations to CasX 515 (SEQ ID NO: 145) systematically
identified from all datasets to improve cleavage
activity at TTC PAM sequences

| | | | Maximum observed | |
|---|---|---|---|---|
| | | | log₂ enrichment | |
| Position | Reference | Alternate | in Ccdb selections | Domain |
| 4 | K | W | 3.51 | OBD-I |
| 5 | R | P | 4.01 | OBD-I |
| 27 | — | P | 4.69 | OBD-I |
| 28 | M | P | 3.69 | OBD-I |
| 56 | Q | P | 3.78 | OBD-I |
| 85 | W | A | 3.96 | helical I-I |
| 102 | — | G | 4.75 | NTSB |
| 104 | — | I | 4.43 | NTSB |
| 104 | — | L | 4.52 | NTSB |
| 130 | S | — | 4.02 | NTSB |
| 151 | Y | T | 3.46 | NTSB |
| 168 | L | D | 3.32 | NTSB |
| 168 | L | E | 4.08 | NTSB |
| 188 | K | Q | 4.96 | NTSB |
| 190 | G | Q | 4.1 | NTSB |
| 223 | G | — | 1.63 | helical I-II |
| 235 | G | L | 4.64 | helical I-II |
| 235 | G | H | 4.97 | helical I-II |
| 239 | S | H | 3.93 | helical I-II |
| 239 | S | T | 4.97 | helical I-II |
| 245 | Q | H | 5 | helical I-II |
| 288 | K | D | 5.08 | helical I-II |
| 288 | K | E | 4.79 | helical I-II |
| 303 | M | R | 3.71 | helical I-II |
| 303 | M | K | 3.29 | helical I-II |
| 307 | L | K | 3.55 | helical I-II |
| 328 | G | R | 3.91 | helical I-II |
| 328 | G | K | 4.58 | helical I-II |
| 334 | — | H | 5.65 | helical II |
| 335 | — | D | 5.5 | helical II |
| 335 | V | P | 5.1 | helical II |
| 345 | — | Q | 5.22 | helical II |
| 441 | — | K | 5.07 | helical II |
| 477 | C | R | 2.94 | helical II |
| 477 | C | K | 3.49 | helical II |
| 502 | S | — | 4.04 | OBD-II |
| 503 | I | R | 3.72 | OBD-II |
| 503 | I | K | Not detected | OBD-II |
| 504 | L | — | 4.24 | OBD-II |
| 542 | R | E | 4.54 | OBD-II |
| 563 | K | — | 3.25 | OBD-II |
| 593 | — | A | 1.83 | OBD-II |
| 610 | K | Q | 3.46 | OBD-II |
| 615 | R | Q | 3.67 | OBD-II |
| 643 | — | A | 2.42 | OBD-II |
| 697 | S | R | 2.67 | RuvC-I |
| 697 | S | K | 2.55 | RuvC-I |
| 906 | V | T | 4.65 | TSL |

### Example 30: Use of Muscle-Specific Promoters to Drive CasX Expression Results in Editing Activity in Muscle Cells and Tissue when the CasX:gRNA System is Expressed from a Transfected AAV Plasmid In Vitro or Packaged and Delivered Via AAVs In Vitro and In Vivo

Experiments were performed to demonstrate that use of muscle-specific promoters to drive CasX expression in an AAV vector results in higher and more selective editing activity in muscle cells than in non-muscle cell types, when the CasX:gRNA system is expressed from an AAV plasmid transfected in vitro. Experiments were also performed to demonstrate that use of muscle-specific promoters to drive CasX expression results in editing at a target locus in muscle cells when the CasX:gRNA system is packaged and delivered via AAVs in vitro and in vivo.

### Materials and Methods

CasX variant 491 and guide scaffold variant 235 were used in these experiments. AAV construct cloning was performed as similarly described in Example 1. Briefly, AAV constructs containing a muscle-specific promoter driving CasX expression and a Pol III U6 promoter driving the expression of gRNA scaffold 235 and a ROSA26-targeting spacer (spacer 35.2; refer to Table 53 for sequences) were generated using standard molecular cloning techniques. Sequence-validated plasmid constructs were midi-prepped for subsequent nucleofection.

TABLE 53
Sequences of AAV constructs used in this example
for testing muscle-specific promoters.

| | | DNA SEQ |
|---|---|---|
| Construct ID | Component Name | ID NO: |
| 215 through 220 | 5′ ITR | 423 |
| | buffer sequence | 788 |
| 215, 220 | UbC promoter | 464 |
| 216 | CK8e promoter | 4051 |
| 217 | MHCK7 promoter | 4052 |
| 218 | Desmin promoter | 4053 |
| 219 | MHCK promoter | 4054 |
| 215 through 220 | buffer sequence | 789 |
| | Kozak | NA |
| | start codon | NA |
| | cMyc NLS | 838 |
| | linker | NA |
| | CasX 491 | 791 |
| | linker | NA |
| | cMyc NLS | 839 |
| | stop codon | NA |
| | buffer sequence | 794 |
| | bGH poly(A) | 514 |
| | buffer sequence | NA |
| | U6 promoter | 661 |
| | buffer sequence | NA |
| | Scaffold 235 | 698 |
| 215 through 219 | Spacer 35.2 | 2920 |
| 220 | Spacer NT | 537 |
| 215 through 220 | buffer sequence | 796 |
| | 3′ ITR | 424 |

* Components are listed in a 5′ to 3′ order within the constructs

Plasmid Nucleofection into Mouse NPCs and Mouse C2C12 Myoblasts:

Briefly, 1 pig of individual AAV plasmids (Table 53) expressing the CasX under the control of different muscle promoters were nucleofected into mouse muscle C2C12 myoblast cells, as well as neuronal NPCs for each experimental condition using methods as described in Example 1. Full media replacement was performed 48 hours post-nucleofections. Five days post-nucleofection, treated cells were harvested for gDNA extraction using the Zymo Quick DNA™ 96 Kit following the manufacturer's instructions. Target amplicons were then amplified from 200 ng of extracted gDNA with a set of primers targeting the mouse ROSA26 locus and processed as described earlier in Example 23 for editing assessment by NGS. As experimental controls, AAV plasmid constructs encoding the following were also tested: 1) UbC promoter driving CasX expression with gRNA containing spacer 35.2; and 2) UbC promoter driving CasX expression with a non-targeting gRNA. A ‘no treatment’ control was also included as an experimental control.

AAV production and AAV titering were performed as described in Example 1.

### AAV Transduction of C2C12 Myoblasts and Myotubes:

AAVs were used to transduce two differentiated states of C2C12 cells—myoblasts and myotubes.

To determine the level of CasX-mediated editing in myoblasts, ˜5,000 C2C12 myoblasts were plated and transduced the next day with AAVs encoding the various CasX:gRNA systems (Table 53) at varying MOIs. Five days following transduction, cells were harvested for gDNA extraction for editing analysis at the ROSA26 locus as described above.

To determine the level of CasX-mediated editing in myotubes, ˜10,000 C2C12 myoblasts were plated and cultured in differentiation media for seven days to induce differentiation into myotubes. After myotube formation, cells were transduced with AAVs encoding the various CasX:gRNA systems (Table 53) at varying MOIs. Five days following transduction, cells were harvested for gDNA extraction for editing analysis at the ROSA26 locus as described above.

### In Vivo Administration of AAVs and Tissue Processing:

˜8E11 AAV viral particles encoding the various CasX:gRNA systems (Table 53) were administered retro-orbitally in C57BL/6J adults. Naïve, untreated mice served as experimental controls. Mice were euthanized at four weeks post-injection. Various tissues were harvested for gDNA extraction using the Zymo Quick DNA/RNA™ miniprep Kit following the manufacturer's instructions. Tissues harvested were skeletal muscles (i.e., tibialis anterior (TA), gastrocnemius (GA), quadriceps (Quad), heart, and diaphragm (DIA)) and non-muscle organs (i.e., liver and lung). Target amplicons were amplified from 200 ng of extracted gDNA with a set of primers targeting the mouse ROSA26 locus and processed as described in Example 18 for editing assessment by NGS. The number of AAV viral genomes (vg) per diploid genome (dg) was determined in the harvested gDNA samples by droplet digital PCR (ddPCR) using the Bio-Rad QX200 Droplet Digital PCR instrument according to standard methods and following the manufacturer's guidelines (see additional detail in Example 34). The vg/dg analysis is an indication of the amount of AAV viral particles delivered into that specific tissue.

### Results:

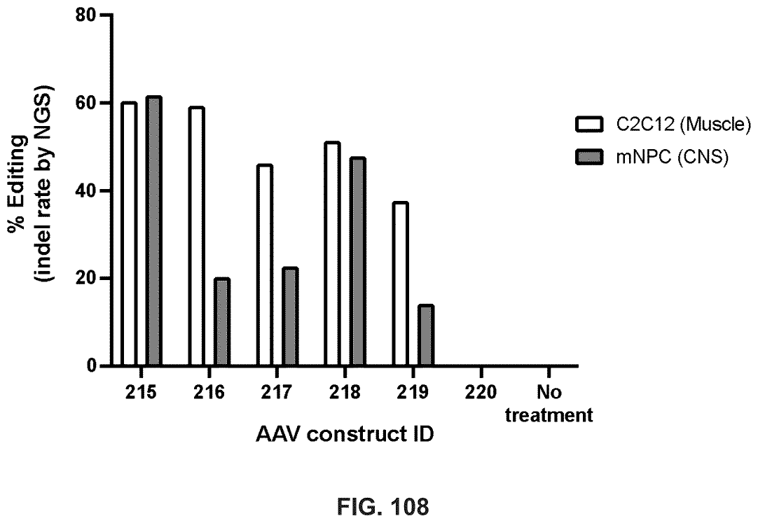

AAV plasmids containing constructs encoding for muscle-specific promoters used to drive CasX expression were nucleofected into C2C12 myoblasts and mouse NPCs to assess the level of specificity of the editing activity in muscle cells compared to neuroprogenitor cells. FIG. 108 shows the quantification of editing measured as indel rate detected by NGS at the mouse ROSA26 locus in C2C12 cells and mouse NPCs for the indicated AAV plasmids. Of the four muscle-specific promoters assessed, use of promoters CK8e (construct ID 216), MHCK7 (construct ID 217), and MHCK (construct ID 219) resulted in higher CasX-mediated editing activity in C2C12 muscle cells compared to that seen in mouse NPCs. Specifically, use of the CK8e promoter resulted in-60% editing at the ROSA26 locus in myoblasts but ˜20% editing in mNPCs, indicating that use of the CK8e promoter would result in more selective expression and higher activity in muscle cell types than neuronal cell types (FIG. 108). Meanwhile, use of the Desmin promoter (construct ID 218) resulted in similar editing levels in both C2C12 cells and mouse NPCs, suggesting minimal tissue-specificity effects when utilizing the Desmin promoter. As anticipated, use of the ubiquitous UbC promoter resulted in similar levels of editing activity in both cell types, while no editing was observed with use of the non-targeting spacer or in the ‘no treatment’ control (FIG. 108).

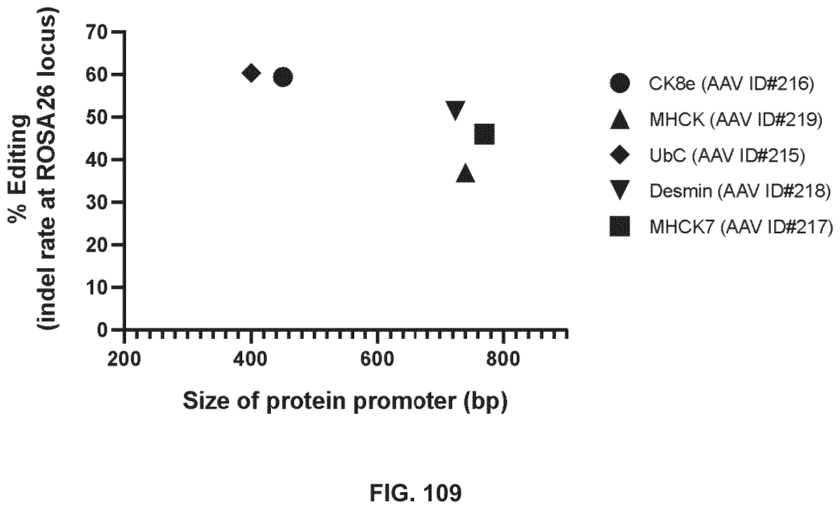

Furthermore, the percent editing at the ROSA26 locus was plotted against the size of the muscle-specific protein promoter, with the results presented in FIG. 109. Of the four tested muscle-specific promoters, the CK8e promoter (construct ID 216) has a similar size of ˜400 bp as that of the UbC promoter and demonstrated a similar level of editing activity.

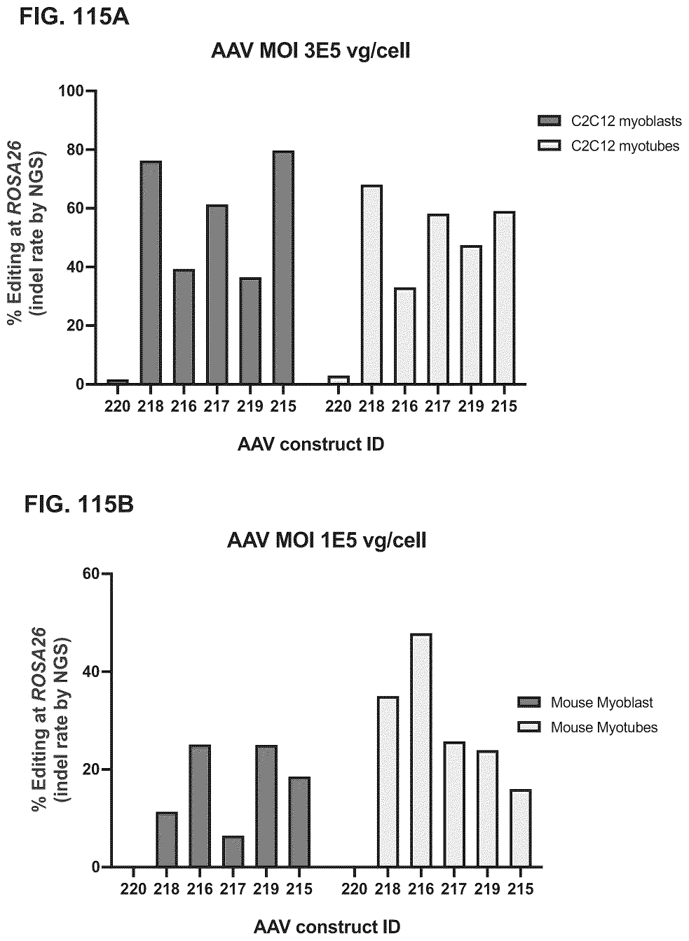

AAVs encoding the CasX:gRNA system, in which muscle-specific promoters were used to drive CasX expression, were used to transduce C2C12 myoblasts and myotubes to assess the level of editing activity in muscle cells at the ROSA26 locus, and the editing results are illustrated in FIGS. 115A and 115B for MOI of 3E5 vg/cell and 1E5 vg/cell respectively. The data demonstrate that use of all four muscle-specific promoters, Desmin, CK8e, MHCK7, and MHCK, were able to induce editing at the ROSA26 locus in both types of muscle cells, albeit at variable levels, when packaged and delivered within AAVs.

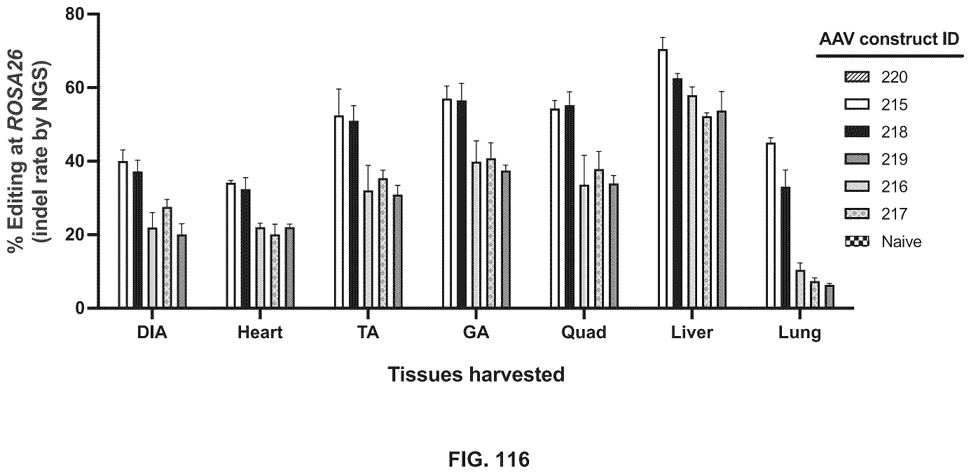

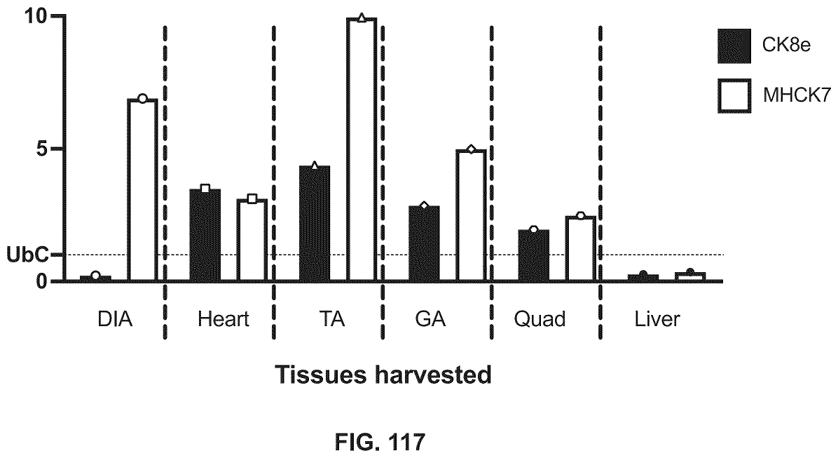

An initial proof-of-concept experiment assessing use of the four different muscle-specific promoters was performed in vivo. AAVs containing CasX protein 491, driven by the muscle-specific promoters or the UbC promoter, and guide scaffold 235 with the ROSA26-targetin spacer were delivered in vivo. Both muscle and non-muscle organs were harvested for editing and vg/dg analyses, depicted in bar graphs in FIG. 116 and FIG. 117 respectively. The data in FIG. 116 show that use of AAVs containing muscle-specific promoters driving CasX expression and a ROSA26-targeting gRNA resulted in varying levels of editing activity across all the harvested tissues. In the muscle tissues (DIA, heart, TA, GA, and Quad), AAVs with the muscle-specific promoters demonstrated lower editing activity compared to that when using the UbC promoter to drive CasX expression. However, in the lung, selective editing activity was detected, such that use of muscle-specific promoters resulted in substantially lower editing activity in the lung compared to that of the UbC promoter, suggesting de-targeting of editing activity in the lung (FIG. 116). The results also show that systemic administration of AAVs using either UbC or muscle-specific promoters induced high editing levels in the liver.

AAV biodistribution was evaluated by quantifying AAV viral particles delivered for a specific tissue using a vg/dg analysis. The vg/dg analysis revealed that similar biodistribution levels were achieved for AAVs containing a muscle-specific promoter or the UbC promoter within a particular tissue (data not shown). Further analysis was performed to determine the relative CasX expression (normalized by vg/dg) driven by muscle-specific promoters CK8e or MHC7 compared to that driven by UbC, and the results are illustrated in FIG. 117. The data demonstrate that after normalizing for delivery to each tissue, use of either muscle-specific promoter CK8e or MHCK7 resulted in higher CasX expression in the muscle tissues relative to the UbC promoter overall, whereas CasX expression in the liver was similar among the promoters compared (FIG. 117). These findings support the significance of using tissue-specific promoters to drive CasX expression within the target tissue to induce editing. The results demonstrate that muscle-specific promoters can be used to drive CasX expression and induce higher editing activity in muscle cell types than in non-muscle cell types when delivered via nucleofection. The data also show that AAVs produced from these AAV plasmids containing muscle-specific promoters were able to induce CasX expression and editing activity in muscle cells in vitro and in vivo when delivered via transduction. The findings also indicate the tissue specificity of using muscle-specific promoters to drive CasX expression compared to use of ubiquitous promoter like UbC. Overall, these findings indicate that muscle-specific promoters can also be utilized in siAAV constructs for delivery and targeting of muscle cells and tissue.

### Example 31: Use of Muscle-Specific Regulatory Elements to Generate AAV Constructs to Produce AAVs that would Transduce and Express CasX More Selectively in Muscle Cells and Tissue

Experiments will be performed to demonstrate that incorporation of muscle-specific regulatory elements, e.g., promoters and enhancers, into AAV plasmids used for AAV production, will result in more selective expression of CasX and higher editing activity in muscle cell types than in non-muscle cell types when the CasX:gRNA system is delivered by AAVs.

### Materials and Methods:

CasX variant 491, 515, 593, 668, 672, 676, or 812 will be used for the experiments described herein. AAV construct cloning, AAV production, and AAV titering will be performed as described in Example 1. Various muscle-specific regulatory elements, e.g., promoters (Table 54) and enhancers (Table 55), will be individually cloned into AAV plasmids harboring sequences encoding for a CasX protein and a gRNA with scaffold 235 and an AAVS1-targeting spacer. The resulting AAV plasmids will be used for AAV production and transduction of human skeletal muscle cells (hSKMCs) to determine editing levels at the AAVS1 locus.

TABLE 54
Sequences of muscle-specific promoters.

| SEQ ID | | DNA SEQ | Promoter |
|---|---|---|---|
| NO: | Promoter | ID NO: | size (bp) |
| 3773 | SP-301 | 4055 | 579 |
| 3774 | Desmin | 4053 | 724 |
| 3775 | CK8e | 4051 | 450 |
| 3776 | MHCK | 4054 | 742 |
| 3777 | MHCK7 | 4052 | 776 |
| 3778 | SpC5-12 | 4056 | 358 |

TABLE 55
Sequences of muscle-specific enhancers.

| SEQ ID | | DNA SEQ | Enhancer |
|---|---|---|---|
| NO: | Enhancer | ID NO: | size (bp) |
| 3779 | Muscle Enhancer 1 | 4057 | 495 |
| 3780 | Muscle Enhancer 2 | 4058 | 344 |
| 3781 | Muscle Enhancer 3 | 4059 | 429 |
| 3782 | Muscle Enhancer 4 | 4060 | 434 |
| 3783 | Muscle Enhancer 5 | 4061 | 171 |
| 3784 | Muscle Enhancer 6 | 4062 | 51 |
| 3785 | Muscle Enhancer 7 | 4063 | 60 |
| 3786 | Muscle Enhancer 8 | 4064 | 41 |
| 3787 | Muscle Enhancer 9 | 4065 | 120 |
| 3788 | Muscle Enhancer 10 | 4066 | 474 |
| 3789 | Muscle Enhancer 11 | 4067 | 519 |
| 3790 | Muscle Enhancer 12 | 4068 | 372 |
| 3791 | MyoD Enhancer | 4069 | 256 |
| 3792 | Cardiac Muscle Enhancer 1 | 4070 | 206 |
| 3793 | Cardiac Muscle Enhancer 2 | 4071 | 255 |
| 3794 | Cardiac Muscle Enhancer 3 | 4072 | 277 |
| 3795 | Cardiac Muscle Enhancer 4 | 4073 | 660 |
| 3796 | Myoblast Muscle Enhancer 1 | 4074 | 22 |
| 3797 | Myoblast Muscle Enhancer 2 | 4075 | 310 |
| 3798 | Myoblast Muscle Enhancer 3 | 4076 | 218 |
| 3799 | Myoblast Muscle Enhancer 4 | 4077 | 353 |
| 3800 | Myoblast Muscle Enhancer 5 | 4078 | 50 |
| 3801 | Skeletal Muscle Enhancer 1 | 4079 | 395 |
| 3802 | Skeletal Muscle Enhancer 2 | 4080 | 382 |
| 3803 | Skeletal Muscle Enhancer 3 | 4081 | 135 |
| 3804 | Skeletal Muscle Enhancer 4 | 4082 | 326 |
| 3805 | Skeletal Muscle Enhancer 5 | 4083 | 273 |
| 3806 | Skeletal Muscle Enhancer 6 | 4084 | 148 |
| 3807 | Skeletal Muscle Enhancer 7 | 4085 | 80 |
| 3808 | Skeletal Muscle Enhancer 8 | 4086 | 437 |
| 3809 | Skeletal Muscle Enhancer 9 | 4087 | 297 |

### AAV Transduction In Vitro:

AAVs will be used to transduce two differentiated states of hSKMCs—myoblasts versus myotubes.

500,000 primary hSKMC cells (ATCC, PCS-950-010) will be plated per 2-4×15 cm dishes in growth media (DMEM/F-12, 20% FBS, 1% PenStrep, 2.5 ng/mL b-FGF). Once cells reach 70% confluency, cells will be lifted and re-seeded in a 96-well plate at 5,000-10,000 cells per well in differentiation media (DMEM, 2% horse serum, 1% PenStrep).

To determine level of CasX-mediated editing in myoblasts, hSKMCs will be transduced with AAVs 4-6 hours after re-seeding in differentiation media at multiple MOIs. Five days following transduction, cells will be harvested for gDNA extraction for editing analysis at the AAVS1 locus by NGS. Briefly, target amplicons will be amplified from 200 ng of extracted gDNA with a set of primers targeting the human AAVS1 locus and processed for NGS as described in Example 23.

To determine the level of CasX-mediated editing in myotubes, re-seeded hSKMCs into differentiation media will continue to be cultured in differentiation media for an additional 7-10 days to promote differentiation into myotubes. After myotube formation, cells will be transduced with AAVs at multiple MOIs. Five days following transduction, cells will be harvested for editing assessment at the AAVS1 locus by NGS as described above.

As a comparison to assess muscle-cell specificity of the produced AAVs, non-muscle cells such as HepG2 hepatocytes or human NPCs will also be transduced with AAVs produced from the same AAV plasmids containing the muscle-specific regulatory elements described herein.

In addition, assessing the incorporation of muscle-specific regulatory elements within an AAV transgene to selectively express CasX in muscle-specific cell types in vivo will also be investigated. These methods for these in vivo experiments are further described in Example 32.

The results of these experiments are expected to demonstrate that AAVs produced from AAV plasmids containing constructs incorporating muscle-specific regulatory elements (promoter and/or enhancer, see Tables 54 and 55) to drive CasX expression, will demonstrate higher editing activity in muscle-specific cell lines compared to non-muscle cell types.

### Example 32: Use of Muscle-Specific AAV Serotypes to Increase Muscle-Specific Cellular and Tissue Tropism to Enhance CasX-Mediated Editing In Vivo

Experiments will be performed to demonstrate that use of muscle-specific AAV serotypes may improve specific cellular and tissue tropism and, therefore, enhance delivery and potency of AAVs in the target muscle cells with minimal editing in off-target cell types in vivo.

### Materials and Methods

AAV plasmid cloning and AAV production and titering will be performed using similar methods described in Example 1. Specifically, the sequences encoding the AAV VP1 serotypes and variants listed in Table 56 will be cloned into relevant pRep/Cap plasmids for use in AAV production.

TABLE 56
Sequences of AAV serotypes to be assessed in vivo.

| AAV serotype | Amino Acid SEQ ID NO: |
|---|---|
| AAV6 | 3810 |
| Rh74 | 3811 |
| RhM4-1 | 3812 |
| AAV9 | 3813 |
| MyoAAV 1A1 | 3814 |
| MyoAAV 1A2 | 3815 |
| MyoAAV 2A | 3816 |

### In Vivo Administration of AAVs and Tissue Processing:

A dose response experiment will be performed, where ˜1E9 to 1E12 AAV viral particles containing CasX protein 491, 515, 672, or 676 and guide scaffold variant 235 with spacer 35.2 targeting the safe harbor ROSA26 locus will be administered retro-orbitally in C57BL/6J adults. Naïve, untreated mice will serve as experimental controls. Mice will be euthanized at different time points, up to four weeks post-injection. Various tissues, including skeletal muscles (e.g., tibialis anterior, gastrocnemius, soleus, quadriceps, heart, and diaphragm) and other organs (liver, spleen, lung etc.) will be harvested for gDNA extraction using the Zymo Quick DNA/RNA™ miniprep Kit following the manufacturer's instructions. Target amplicons will then be amplified from 200 ng of extracted gDNA with a set of primers targeting the mouse ROSA26 locus and processed as described earlier in Example 18 for editing assessment by NGS.

Results from the experiments are expected to show that AAVs containing CasX protein and guide scaffold 235 with the ROSA26-targeting spacer will be able to edit the target ROSA26 locus in various muscle tissues. Furthermore, it is expected that higher editing activity will be detected in muscle tissues compared to that detected in other tissues, such as the liver or spleen, which would indicate the ability to increase muscle-specific tissue tropism in vivo by incorporating constructs encoding for muscle-specific AAV serotypes into the pRep/Cap plasmid.

### Example 33: AAV-Mediated Selective Expression of CasX in Photoreceptors Result in Strong On-Target Activity In Vivo by NGS and Structural Analysis

Experiments were conducted to demonstrate the ability of CasX to edit selectively in the photoreceptors in the mouse retina by restricting its expression with a selective photoreceptor promoter, with a spacer targeting the P23 residue at a therapeutically relevant level in the wild-type retina. We further show strong correlation between editing and proteomic levels in a transgenic reporter model expressing GFP only in rod photoreceptors. Here, we assessed whether CasX variant 491 and guide variant 174 with a spacer targeting the integrated GFP locus generated significant, detectable editing levels in the retina when injected subretinally and evaluated the efficacy of two different viral doses (1.0e+9 and 1.0e+10 vg per eye).

### Methods:

### Generation of AAV Plasmids and Viral Vectors:

The CasX variant 491 under the control of the various photoreceptor-specific promoters (RP1, RP2, RP3 based on endogenous rhodopsin RHO promoter, and RP4, RP5 based on endogenous G-coupled Retinal Kinase GRK1 promoter; sequences in Table 57), as well as the CMV promoter, and the gRNA guide variant 174/spacer 11.30 (AAGGGGCUCCGCACCACGCC; SEQ ID NO: 4088), targeting mouse RHO exon 1 at P23 residue) under the U6 promoter were cloned into pAAV plasmid flanked with AAV2 ITR. A WPRE sequence was also included in the p59.RP4.491.174.11.30 and p59.RP5.491.174.11.30 plasmids. For the efficacy study in the Nrl-GFP model, spacer 4.76 (UGUGGUCGGGGUAGCGGCUG; SEQ ID NO: 4089) targeting GFP was cloned into AAV-cis plasmid p59.RP1.491.174 using standard cloning methods.

TABLE 57
Rho promoter sequences.

| Promoter | PR construct | DNA SEQ ID NO: |
|---|---|---|
| RHO | RP1 | 4090 |
| RHO535-CAG | RP2 | 4091 |
| RHO-intron | RP3 | 4092 |
| GRK1 | RP4 | 4093 |
| GRK1-SV40 | RP5 | 4094 |
| GRK1-CAG | RP6 | 4095 |

AAV production and AAV titering were performed as described in Example 1.

The AAV vector AAV.RP1.491.174.4.76 was produced at the University of North Carolina (UNC) Vector Core using the triple transfection methods in HEK239T.

### Subretinal Injections:

Subretinal injections were performed on 4-5-week-old C57BL/6J mice and heterozygous Nrl-GFP/C57BL/5J mice (Jackson Laboratories). Briefly, mice were anesthetized, and proparacaine was applied topically on the cornea, and the eyes were dilated with drops of tropicamide and phenylephrine. Eyes were kept lubricated with GenTeal gel during surgery. Under a surgical microscope, an ultrafine 30½-gauge disposable needle was passed through the sclera, at the equator and next to the limbus, to create a small hole into the vitreous cavity. Using a blunt-end needle, 1-1.5 μL of virus was injected directly into the subretinal space, between the RPE and retinal layer. Each mouse from the experimental groups was injected in one eye with 1.0e+9, 5.0e+9 or 1.0e+10 vg per eye, and the contralateral eye injected with the AAV formulation buffer.

### Western Blot:

To generate protein lysates, eyes were freshly enucleated and dissected in ice-cold PBS, snap-frozen in dry ice, and resuspended in RIPA buffer (150 mM NaCl, 1% NP40, 0.5% deoxycholate, 0.1% SDS, 50 mM Tris pH8.0, dH20) freshly supplemented with protease inhibitors (5 mg/mL final concentration), DTT and PMSF (final concentration 1 mM respectively) in individual 1.5 mL Eppendorf tube per retina. Retinal tissue was further homogenized in small pieces using a RNA-free disposable pellet pestles (Fisher scientific, #12-141-364) and incubated on ice for 30 minutes, flipping the tube occasionally to gently mix. Samples were then centrifuged at 4° C. at full speed for 20 minutes to pellet genomic DNA. Protein extracts and gDNA cell pellets were then separated. For protein extracts, supernatants were collected. Protein concentrations were determined by BCA assay and read on Tecan plate reader. 15 μg of total protein lysate of mouse retina were separated by SDS-PAGE (Bio-Rad TGX gels) and transferred to polyvinylidene difluoride membranes using the Transblot Turbo. The membranes were blocked with 5% nonfat dry milk for 1 h at room temperature and incubated overnight at 4° C. with the primary antibody. Then, blots were washed with Tris-buffered saline with the Tween-20 (137 mM sodium chloride, 20 mM Tris, 0.1% Tween-20, pH 7.6) for three times and incubated with the horseradish peroxidase-conjugated anti-rabbit or anti-mouse secondary antibody for 1 hour at room temperature. After washing three times, the membranes were developed using Chemiluminescent substrate ECL and imaged on the ChemicDoc (X). Blot images were processed with ImageLab.

### Tissue Processing and NGS Analysis:

Animals were sacrificed, and the eyes enucleated in fresh PBS. Whole retinae were isolated from the eye cups and processed for gDNA extraction as described previously in the western blot section. Genomic gDNA pellets were processed with the DNeasy Blood & Tissue Kit (Qiagen®) according to the manufacturer's instructions. Amplicons were amplified from 200 ng of gDNA with a set of primers targeting the genomic region of interest. Amplicons were bead-purified (Beckman coulter, Agencourt Ampure XP) and then re-amplified to incorporate Illumina™ adapter sequence. Specifically, these primers contained an additional sequence at the 5′ ends to introduce Illumina™ read and 2 sequences as well as a 16 nt random sequence that functions as a unique molecular identifier (UMI). Quality and quantification of the amplicon was assessed using a Fragment Analyzer DNA analyzer kit (Agilent, dsDNA 35-1500 bp). Amplicons were sequenced on the Illumina™ Miseq™ according to the manufacturer's instructions. Raw fastq files from sequencing were processed as follows: (1) the sequences were trimmed for quality and for adapter sequences using the program cutadapt (v. 2.1); (2) the sequences from read 1 and read 2 were merged into a single insert sequence using the program flash2 (v2.2.00); and (3) the consensus insert sequences were run through the program CRISPResso2 (v 2.0.29), along with the expected amplicon sequence and the spacer sequence. This program quantifies the percent of reads that were modified in a window around the 3′ end of the spacer (30 bp window centered at −3 bp from 3′ end of spacer). The activity of the CasX molecule was quantified as the total percent of reads that contain insertions, substitutions and/or deletions anywhere within this window.

### Immunohistology:

Enucleated eyes were placed in 10% formalin overnight at 4° C. Retinae were dissected out from the eye cups, rinsed in PBS thoroughly and immersed in 15%-30% sucrose gradient. Tissues were embedded in optimal cutting temperature (OCT), frozen on dry ice before being transferred to −80° C. storage. 20 μM sections were cut using a cryostat. The sections were blocked for >1 hour at room temperature in the blocking buffer (2% normal goat serum, 1% BSA, 0.1% Triton-X 100) before antibody labeling. The antibodies used were: anti-mouse HA (abcam, 1:500); Alexa Fluor 488 rabbit anti-mouse (Invitrogen™, 1:2000). Slides were counterstained with Hoechst 33342 (Thermo Fisher Scientific™, Hemel Hempstead, UK) and mounted with Prolong Diamond antifade mounting medium (Thermo Fisher Scientific™, Hemel Hempstead, UK). Confocal fluorescence imaging was subsequently performed using the LSM-710 inverted confocal microscope system (Carl Zeiss, Cambridge, UK).

### Results:

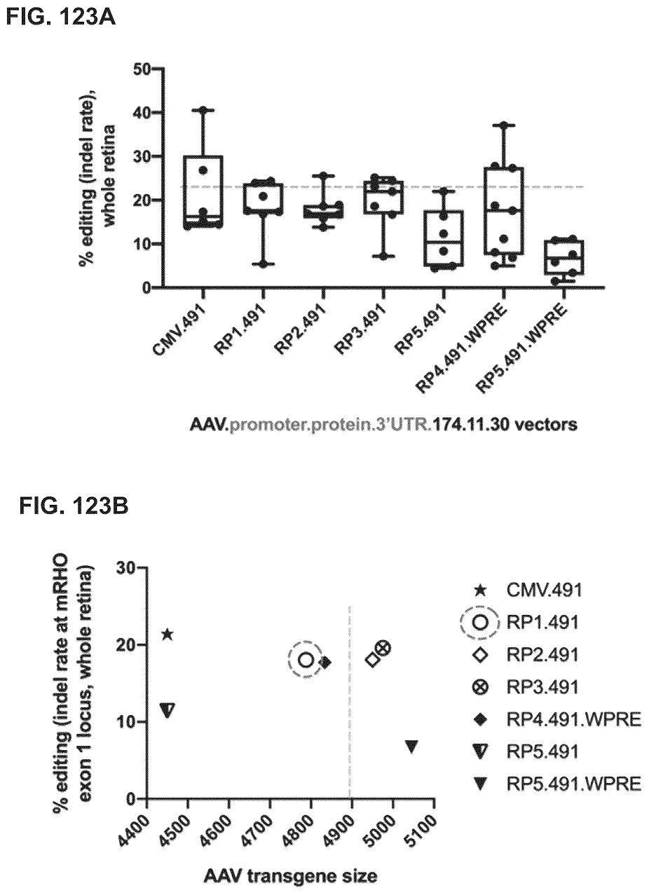

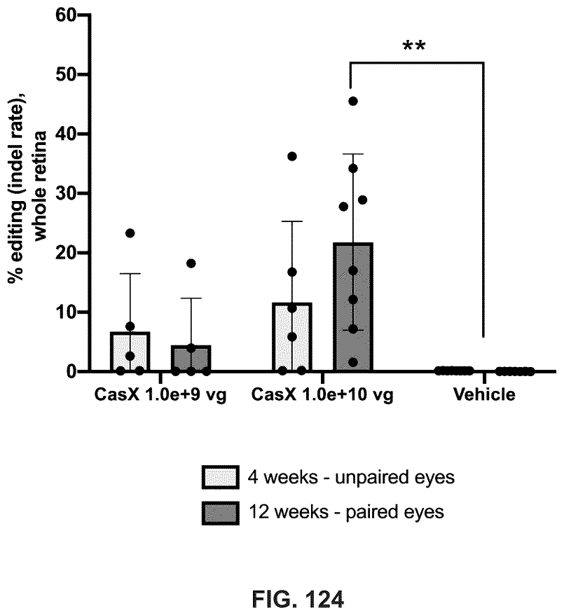

Editing levels were quantified at the mRHO exon locus in 3-week-old C57BL/6J that were injected subretinally with AAV vectors expressing CasX 491 under the control of multiple engineered retinal and ubiquitous promoters to identify promoters driving strong levels of editing in the photoreceptors, with spacer 11.30. Rod-specific RP1, RP2, RP3, RP4 promoters mediated very similar levels of editing (˜20%). Vectors AAV.RP5.491.174.11.30 and AAV RP5.491.WPRE.174.11.30 led to lower expression levels (˜10 and 8% respectively, FIG. 123A). We identified optimized vectors AAV.RP1.491.174.11.30 as most potent vectors for further functional and distribution study, with the goal of achieving high levels of editing in vivo in photoreceptors as well as making the transgene plasmid significantly smaller in size to package within the AAV (100-400 bp shorter than other constructs with similar level of activity (FIG. 123B). This optimized construct was further validated by conducting an efficacy study in a transgenic model expressing GFP in rod photoreceptors, a convenient model used in the field to validate rod-specific or knock down of protein. AAV.RP1.491.174.4.76 vectors were injected at 2 different doses to study efficacy. 4 and 12-weeks post-injections, editing levels at the integrated GFP locus were quantified by NGS, and detectable editing levels were observed. With the 1.0E+9 vg/eye dose arm, we observed ˜8% of editing levels. With the increased dose group injected with 1.0e+10 vg, 10% editing levels were detectable at 4-weeks, which increased by 2-fold in the follow-up time point, 12-weeks post-injections (FIG. 124).

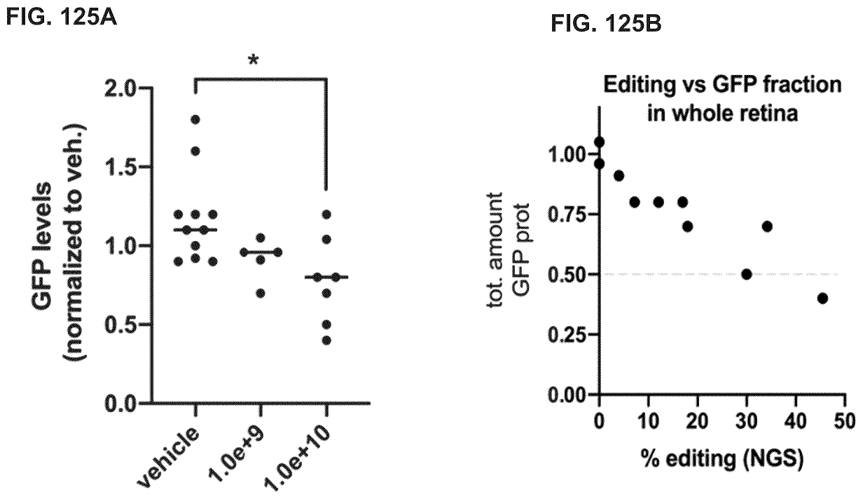

Editing levels were confirmed by structural and proteomic analysis. Western blot analysis of 12-week post-injection retinal lysates showed strong correlation between levels of editing and reduction in GFP protein (FIG. 125B), with protein knock-down detected with as low as 5% editing in whole-retina. GFP protein levels were significantly lower than the vehicle group in the AAV-CasX-treated retinas at the 1.0e+10 vg/eye dose (FIG. 125A).

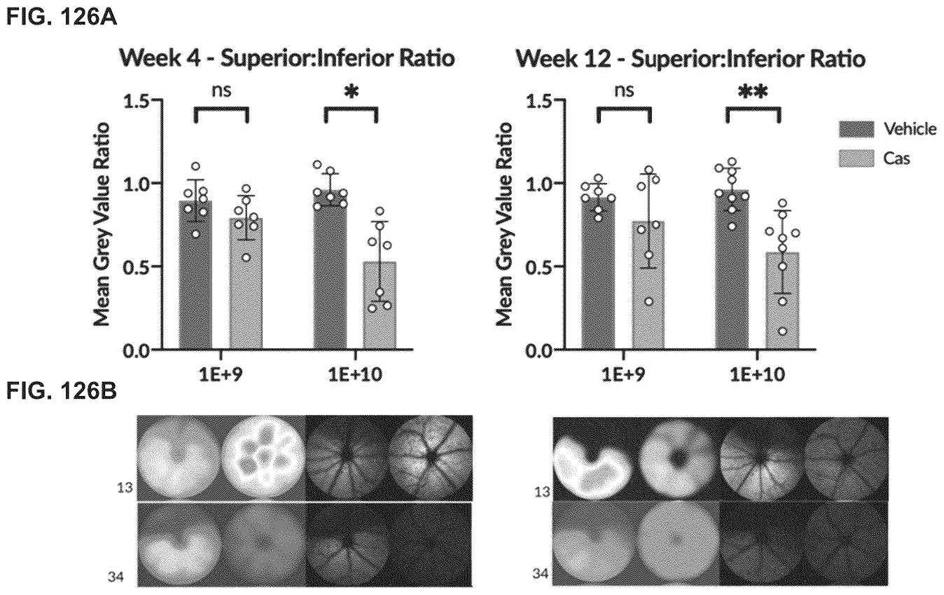

These results were also confirmed by in vivo fundus imaging of GFP fluorescence. The ratio of superior to inferior retina mean grey values showed a reduction in 20% and 50% GFP fluorescence by week 12 (FIG. 126A). A complete decrease in GFP fluorescence over time was visible within the quadrant who received the subretinal injection only in the injected retinas compared to the vehicle group (FIG. 126B).

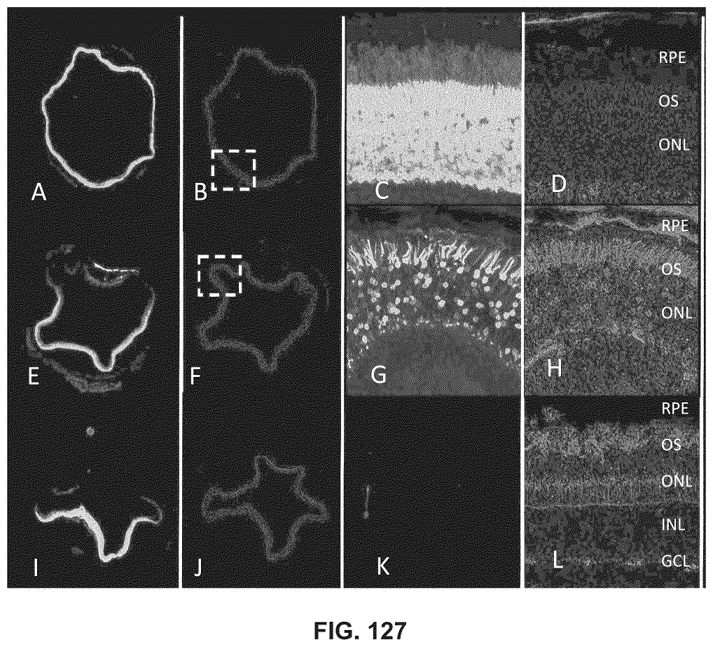

Immunochemistry staining confirmed (FIG. 127) the decrease of GFP protein expression in rod photoreceptors. Representative confocal images show strong GFP expression in the retinae injected with only the AAV formulation buffer. Whole retina is expressing GFP, matching with the nuclei staining (panels A-C of FIG. 127). No HA expression was detectable, as a read-out of AAV-mediated CasX transgene expression (panel D of FIG. 127). Retinae injected with 1.0e+9 and 1.0e+10 showed strong decrease in GFP expression in whole retina sections, in a dose-dependent manner (panels E-L of FIG. 127), which correlated with detectable levels of HA only rod outer segments (OS) and outer nuclear layers (ONL), confirming the promoter RP1 selectivity for rod photoreceptors. High dose treatment resulted in complete knockdown of injected retina (˜50% of GFP knockdown in whole-retina, as injection is limited to the superior gradient) while the 1.0e+9 vg dose decreased ˜50% of GFP expression in localized area (panels G and K of FIG. 127) compared to control (panel C of FIG. 127).

The results demonstrate proof-of-concept that CasX with a gRNA targeting the mouse P23 RHO locus can achieve therapeutically-relevant editing levels at the mouse P23 locus when only expressed in rod-photoreceptors, the therapeutic cell target, via AAV-mediated subretinal delivery. Furthermore, the specificity and efficacy of the vector were demonstrated by conducting a follow-up study targeting a GFP locus integrated in a reporter model overexpressing GFP in photoreceptors in which the results show a strong correlation between editing levels and protein knock-down assessed by western blot, fundus imaging and histology.

### Example 34: AAV-Mediated Selective Expression of CasX in Rod and Cone Photoreceptors Results in Strong On-Target Activity at a Safe Harbor Locus in the Murine Retinae

Experiments were performed to demonstrate the ability of CasX to edit selectively rod and cone photoreceptors in the mouse retina by restricting its expression with a selective photoreceptor promoter, with a gRNA spacer targeting a safe harbor locus in the mouse genome. The correlation between editing and proteomic levels was demonstrated in a transgenic reporter mouse model that expressed GFP only in the rod photoreceptors.

### Materials and Methods:

### Generation of AAV Plasmids and Viral Vectors:

CasX variant 491, flanked on either side by a c-MYC NLS, under the control of the various photoreceptor-specific promoters (listed in Table 58) based on the endogenous G-coupled Rhodopsin Kinase 1 (GRK1) promoter, and the gRNA guide variant 235 with spacer 35.2 (AGAAGAUGGGCGGGAGUCUU; SEQ ID NO: 4096) targeting the mouse ROSA26 locus under the U6 promoter, were cloned into a pAAV plasmid flanked with AAV2 ITR using standard cloning methods.

TABLE 58
Sequences of GRK1 promoter variants.

| Promoter | DNA SEQ ID NO: |
|---|---|
| GRK1(93) | 4097 |
| GRK1(94) | 4098 |
| GRK1(174) | 4099 |
| GRK1(199) | 4100 |
| GRK1(241) | 4101 |
| GRK1(292) | 4102 |
| GRK1(292)-SV40 | 4103 |
| GRK1-SV40 | |

AAV production and AAV titering were performed as described in Example 1.

Subretinal injections were performed in C57BL/6J mice as described in Example 33. Each mouse from the experimental groups was injected in one eye with 5E8 vg per eye. AAVs containing the GRK1-SV40 with a non-targeting (NT) gRNA served as an experimental control.

The processing of tissues, which were harvested three weeks post-injection, and subsequent NGS analysis were performed as described in Example 33. Briefly, gDNA was extracted using the Zymo Quick DNA/RNA™ miniprep Kit following the manufacturer's instructions and used for the amplification of the target amplicon at the ROSA26 locus. Target amplicons were sequenced and processed as described in Example 33.

ddPCR Analysis of AAV Genomes (Vg/Dg):

The number of AAV viral genomes (vg) per diploid genome (dg) was determined in gDNA samples extracted from harvested tissues by ddPCR using the Bio-Rad QX200 Droplet Digital PCR instrument according to standard methods and following the manufacturer's protocol and guidelines. Briefly, ddPCR reactions containing the extracted gDNA samples were set up, serially diluted, and subjected to droplet formation using the droplet generator. Within each droplet, a PCR amplification reaction was performed using a primer-probe set specific to CasX, an indicator of the transgene, and mouse RPP30, an indicator of the mouse genome. Subsequently, droplet fluorescence was determined using the QX200 Droplet Reader with the Bio-Rad QuantaSoft software. To calculate total vg/dg for each tissue, the total quantified copy amount for CasX was divided by the copy amount calculated for RPP30, and then divided by 2 (diploid genome per cell).

### Results:

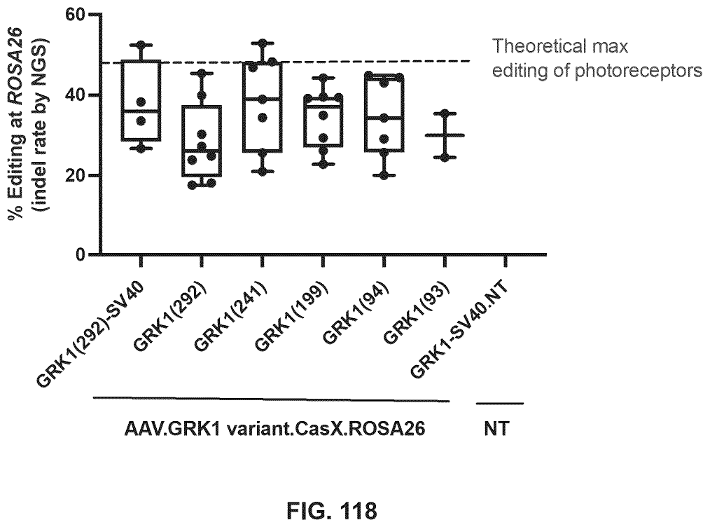

Editing levels at the ROSA26 locus were quantified in retinae harvested from mice injected subretinally with AAVs expressing CasX 491 under the control of various engineered retinal promoters (listed in Table 58) to identify promoters driving the strongest levels of CasX-mediated editing in the photoreceptors. FIG. 118 is a box plot that shows the quantification of these editing levels at the ROSA26 locus for the indicated GRK1 promoter variants. The data demonstrate that use of the GRK1 promoter variants to drive CasX expression resulted in similar levels of editing (˜30-38%). Of the promoter variants tested, AAVs containing the GRK1(292)-SV40 and GRK1(241) promoter variants yielded the highest average editing levels, achieving 37.73±10.89% and 38.27±11.98% editing respectively (FIG. 118). As illustrated in FIG. 118, use of GRK1(292)-SV40 and GRK1(241) promoters resulted in the maximum editing that could be achieved in the photoreceptors (dashed line, which indicates the theoretical maximum editing of photoreceptors that can be achieved with optimal transduction).

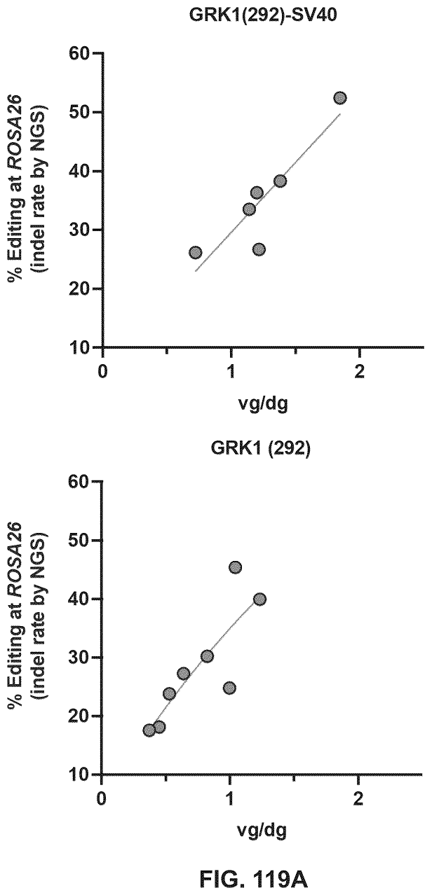

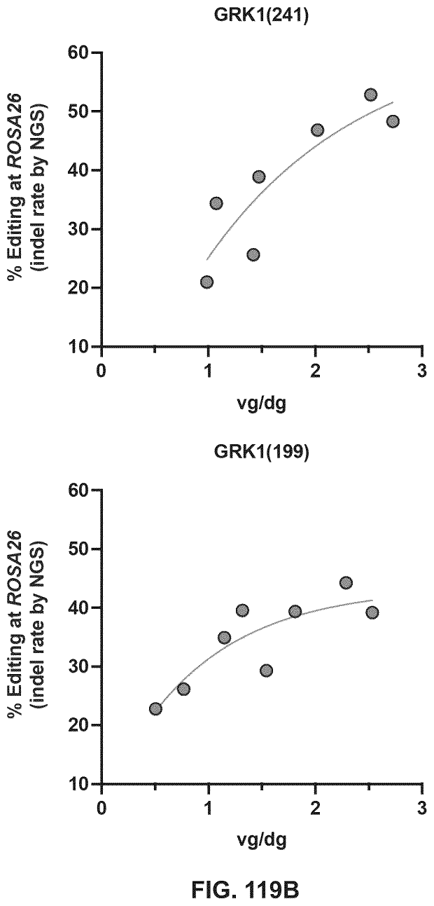

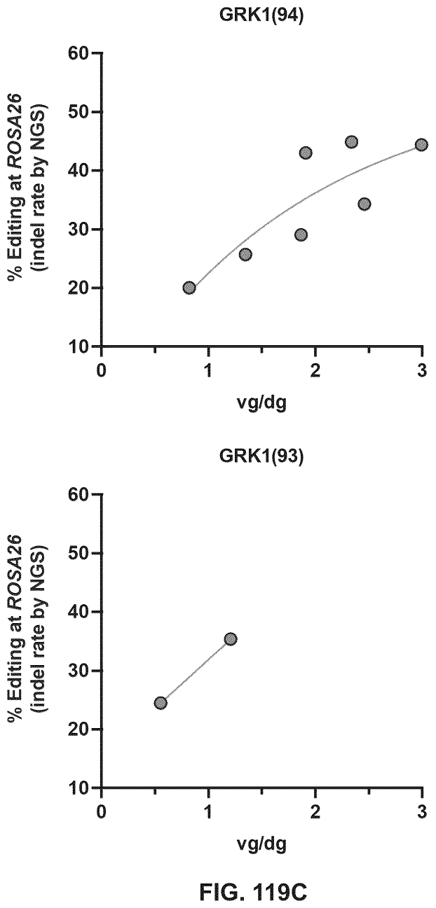

Additional analyses were performed by correlating editing levels achieved when using a particular promoter variant with the vg/dg quantification, to account for potential variation in AAV delivery. The editing profile for each promoter variant was plotted with the corresponding vg/dg value, and a nonlinear regression curve was fitted to assess the correlation (FIGS. 119A-119C). The data demonstrate that there is an overall positive correlation between AAV dose (vg/dg) and percent editing, such that higher amount of AAV delivered would correlate with higher editing. Slope values were calculated for each regression plot, and the calculations are displayed in Table 59. Analysis of the slope values revealed that an increase from 1 vg/dg to 2 vg/dg for AAVs containing the GRK1(292)-SV40 or GRK1(292) promoter resulted in the highest incremental change in editing levels compared to the incremental changes achieved for the shorter promoter variants (Table 59, FIGS. 119A-119C). Furthermore, higher variability in editing levels was observed with use of the shorter promoter variants, especially with use of GRK1(199) and GRK1(94), indicated by the higher standard deviation values calculated for the corresponding slopes of the curves (Table 59, FIGS. 119A-119C). The data also show that saturation in editing was achieved when >1.5 vg/dg, given the flattening of the curve observed (FIGS. 119A-119C). Interestingly, use of the GRK1(93) promoter appeared to exhibit stronger editing kinetics compared to the GRK1(199) and GRK1(94) promoters, given the higher slope value observed (Table 59).

TABLE 59
Slope values calculated from the nonlinear
regression curves in FIGS. 119A-119C.

| Promoter | Slope value | Standard deviation of slope |
|---|---|---|
| GRK1(292)-SV40 | 23.78 | 5.809 |
| GRK1(292) | 26.65 | 8.124 |
| GRK1(241) | 15.17 | 11.78 |
| GRK1(199) | 8.792 | 21.35 |
| GRK1(94) | 11.44 | 12.02 |
| GRK1(93) | 16.6 | 15.31 |

The results from these experiments demonstrate proof-of-concept that CasX, driven by the various photoreceptor-specific promoters with the targeting gRNA, can achieve editing in the photoreceptor cells of the retinae when delivered by AAVs via subretinal administration. Variable levels of editing were achieved when using the different promoter variants, suggesting that stronger promoters can be used to drive higher CasX expression to achieve therapeutic outcomes, while weaker promoters may be used as a strategy for tuning CasX expression and consequent editing activity within the context of self-inactivating AAVs. Furthermore, given the limited cargo capacity of the AAV transgene, use of a shorter tissue-specific promoter to drive sufficient CasX expression to induce editing would be especially beneficial in the context of a dual-guide AAV vector.

### Example 35: In Vivo Administration of the siAAV-CasX (siAAV) System Results in Efficient Editing at a Target Locus while Inducing CasX Self-Inactivation In Vivo

Experiments were performed to measure genome editing and siAAV depletion in vivo in mice administered siAAVs with zero, one, or two STALL sites.

### Materials and Methods

siAAVs encoding CasX variant 491 with guide scaffold 235 and spacers targeting either the ROSA26 locus or a non-targeting control were used in these in vivo experiments. Specifically, siAAV constructs were engineered such that there was either one or two STALL sites adjacent to the CasX coding sequence. In the constructs with one STALL site, there was one ROSA26 protospacer sequence 5′ of the CasX coding sequence, preceded by an ATCN, CTCN, or GTCN PAM (siAAV constructs 146, 159, and 160, respectively). In the constructs with two STALL sites, the CasX coding sequence was flanked on both the 5′ and 3′ end by a ROSA26 protospacer sequence, which were both preceded by either an ATCN, CTCN, or GTCN PAM (siAAV constructs 161, 162, and 163, respectively). As controls, constructs with one or two ATCN STALL sites were used with sgRNAs targeting the AAVS1 locus (siAAV constructs 164 and 165, respectively). The AAVS1 locus was not present in the mice used in this experiment; accordingly, these constructs served as negative controls in which the target of the sgRNA did not match the identity of the protospacer sequence at the STALL site. Finally, constructs without STALL sites with sgRNAs targeting either ROSA26 or AAVS1 were used (siAAV constructs 140 and 166, respectively). A summary of the constructs is provided in Table 60, below. The nucleic acid sequences of the constructs are provided in Table 61. AAV constructs were cloned and AAVs were produced using adherent HEK293T cells as described in Example 1.

TABLE 60
Description of siAAV constructs with
zero, one, or two STALL sites

| | Number | | | | SEQ ID NO |
|---|---|---|---|---|---|
| siAAV | of | STALL | | | (full-length |
| Construct | STALL | site | Target of | Spacer | construct |
| ID | sites | PAM | sgRNA | ID | sequence) |
| 140 | 0 | n/a | ROSA26 | 35.2 | 4141 |
| 146 | 1 | ATCN | ROSA26 | 35.2 | 4142 |
| 159 | 1 | CTCN | ROSA26 | 35.2 | 4143 |
| 160 | 1 | GTCN | ROSA26 | 35.2 | 4144 |
| 161 | 2 | ATCN | ROSA26 | 35.2 | 4145 |
| 162 | 2 | CTCN | ROSA26 | 35.2 | 4146 |
| 163 | 2 | GTCN | ROSA26 | 35.2 | 4147 |
| 164 | 1 | ATCN | AAVS1 | 31.63 | 4148 |
| | | | (“non-targeting”) | | |
| 165 | 2 | ATCN | AAVS1 | 31.63 | 4149 |
| | | | (“non-targeting”) | | |
| 166 | 0 | n/a | AAVS1 | 31.63 | 4150 |
| | | | (“non-targeting”) | | |

TABLE 61
Sequences of siAAV constructs with
zero, one, or two STALL sites

| siAAV | | |
|---|---|---|
| Construct | | DNA SEQ |
| ID | Component Name | ID NO: |
| 140, 166 | 5′ ITR | 423 |
| | buffer sequence | 788 |
| | UbC promoter | 464 |
| | buffer sequence | 789 |
| | Kozak | NA |
| | start codon | NA |
| | 5′ c-MYC NLS | 838 |
| | Linker | NA |
| | CasX 491 | 791 |
| | Linker | NA |
| | 3′ c-MYC NLS | 2985 |
| | Stop codon | NA |
| | buffer sequence | 794 |
| | bGH poly(A) signal | 514 |
| | buffer sequence | NA |
| | U6 promoter | 661 |
| | buffer sequence | NA |
| | Scaffold 235 | 698 |
| | Spacer | |
| | buffer sequence | 796 |
| | 3′ ITR | 424 |
| 146, 159, | 5′ ITR | 423 |
| 160, 164 | buffer sequence | 788 |
| | UbC promoter | 464 |
| | buffer sequence | 789 |
| | STALL site 1 | |
| | Kozak | NA |
| | start codon | NA |
| | 5′ c-MYC NLS | 838 |
| | Linker | NA |
| | CasX 491 | 791 |
| | Linker | NA |
| | 3′ c-MYC NLS | 2985 |
| | Stop codon | NA |
| | buffer sequence | 794 |
| | bGH poly(A) signal | 514 |
| | buffer sequence | NA |
| | U6 promoter | 661 |
| | buffer sequence | NA |
| | Scaffold 235 | 698 |
| | Spacer | |
| | buffer sequence | 796 |
| | 3′ ITR | 424 |
| 161-163, | 5′ ITR | 423 |
| 165 | buffer sequence | 788 |
| | UbC promoter | 464 |
| | buffer sequence | 789 |
| | STALL site 1 | — |
| | Kozak | NA |
| | start codon | NA |
| | 5′ c-MYC NLS | 838 |
| | Linker | NA |
| | CasX 491 | 791 |
| | Linker | NA |
| | 3′ c-MYC NLS | 2985 |
| | Stop codon | NA |
| | buffer sequence | 4104 |
| | STALL site 2 | — |
| | buffer sequence | 4105 |
| | bGH poly(A) signal | 514 |
| | buffer sequence | NA |
| | U6 promoter | 661 |
| | buffer sequence | NA |
| | Scaffold 235 | 698 |
| | Spacer | — |
| | buffer sequence | 796 |
| | 3′ ITR | 424 |
| 140, 146, | ROSA26 spacer 35.2 | 2920 |
| 159-163 | | |
| 164-166 | AAV-S1 | 4106 |
| | (“non-targeting”) spacer 31.63 | |
| 146, 161, | ATCN STALL site | 2984 |
| 164-165 | | |
| 159, 162 | CTCN STALL site | 4107 |
| 160, 163 | GTCN STALL site | 4108 |

* Components are listed in a 5′ to 3′ order within the constructs

Briefly, 8E9 siAAV particles were administered intracerebroventricularly into C57Bl/6 P0-P1 neonates. Three weeks and 16 weeks post-injection, mice were euthanized by terminal anesthesia followed by transcardiac perfusion. Brains and (at the 16 week timepoint) liver were harvested and RNA and gDNA was extracted using the Zymo Quick-DNA/RNA™ miniprep kit according to the manufacturer's instructions. RNA and gRNA were also extracted from a naïve mouse, as a control. The abundance of viral genomes per diploid genome (vg/dg) was determined as an indication of the number of siAAV genomes per mouse cell.

### Assessment of RNA Levels by RT-qPCR:

RNA extracted from brain tissue was used as input for reverse transcription. The resulting cDNA served as input for qPCR reactions to quantify the amount of transcribed CasX and guide scaffold 235 using HEX/FAM-based detection with primers-probe sets targeting CasX or guide scaffold 235. Expression of the ACTB housekeeping gene was used for normalization. Expression data were analyzed according to the double delta Ct method. Statistical significance was calculated using a 1-way ANOVA with Dunnett's Multiple Comparison test.

### Assessment of ROSA26 Editing by NGS:

Target amplicons were amplified from extracted gDNA with a set of primers targeting the mouse ROSA26 locus and processed for NGS as described in Example 23.

### Results:

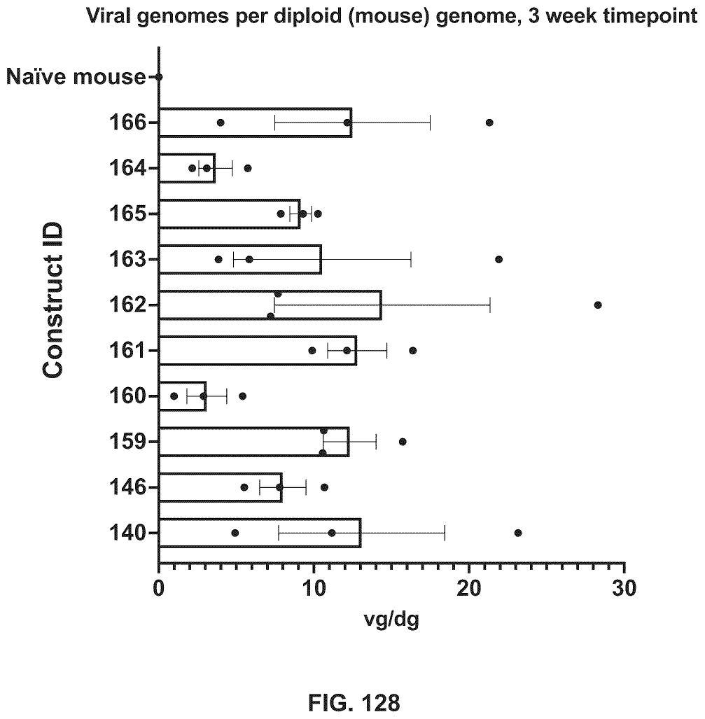

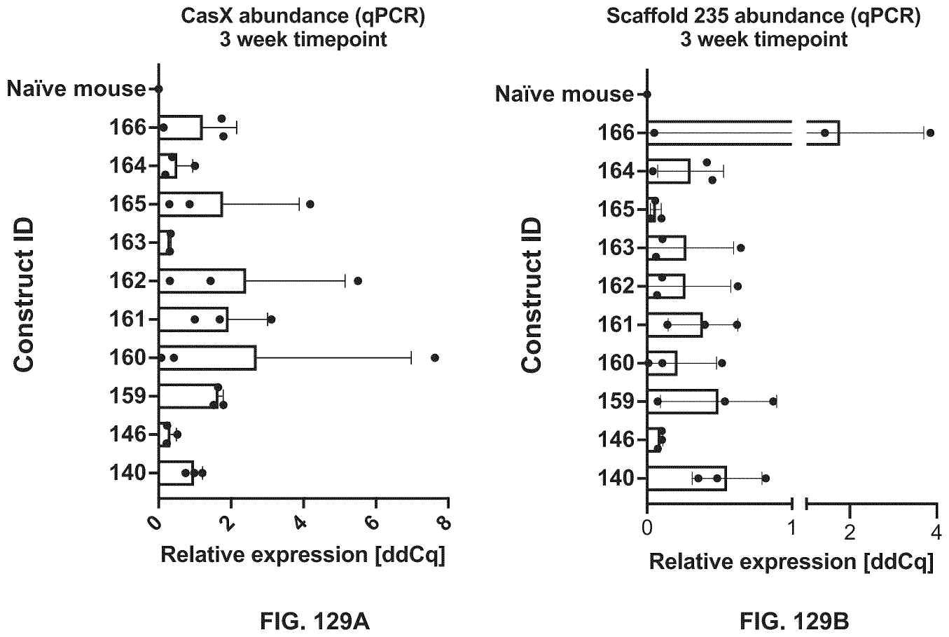

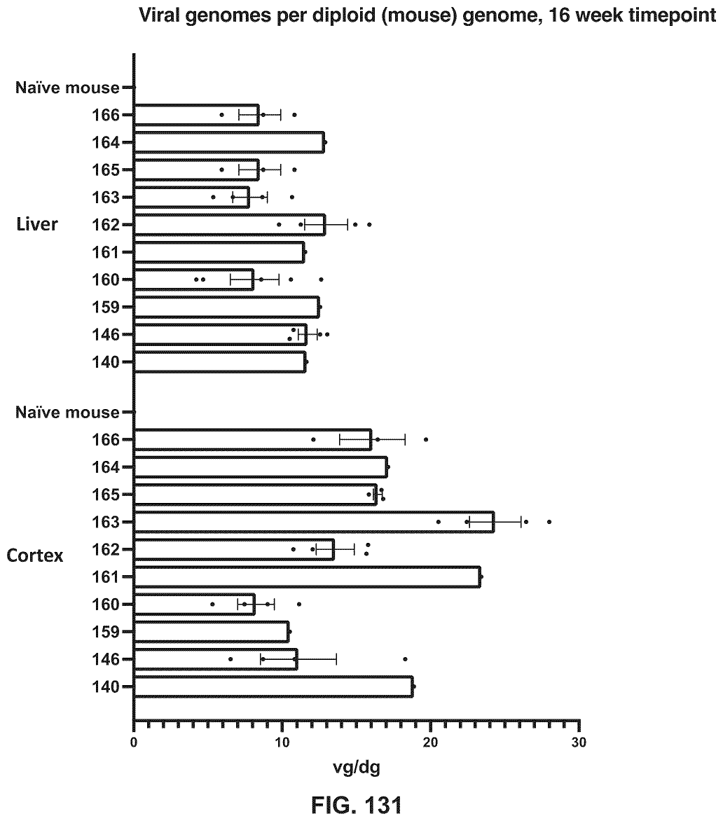

siAAV vectors with zero, one, or two STALL sites were administered to mice, and the level of AAV genome per mouse cell, mRNA encoding CasX, guide scaffold 235, and editing of the ROSA26 locus was measured at three weeks and 16 weeks following administration. Three weeks following siAAV administration, viral genomes, RNA encoding CasX, and guide scaffold 235 were detectable in all samples except for samples from the naïve mouse (see FIGS. 128, 129A, and 129B). The vg/dg, level of CasX mRNA, and level of sgRNA varied between animals. At 16 weeks following administration, viral genomes were found in the liver and cortex for all constructs tested (FIG. 131).

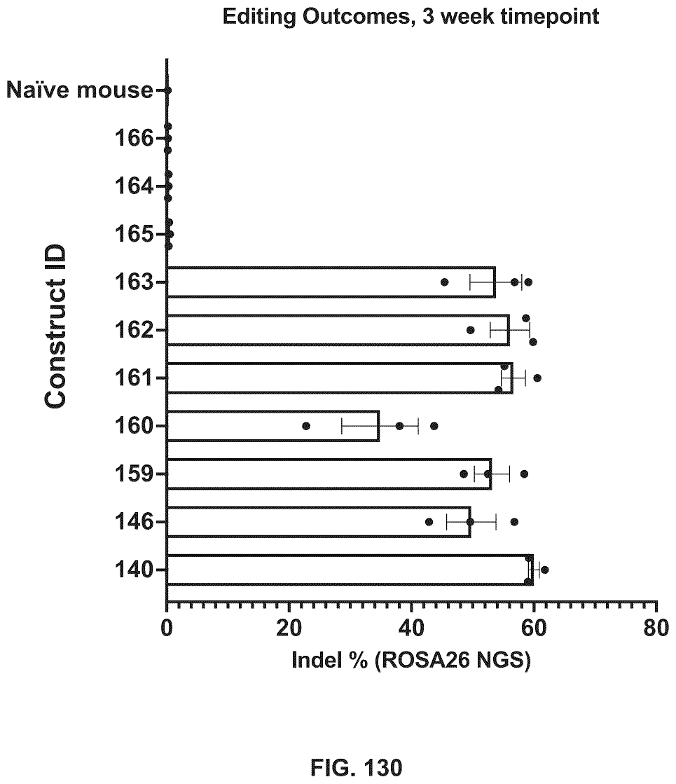

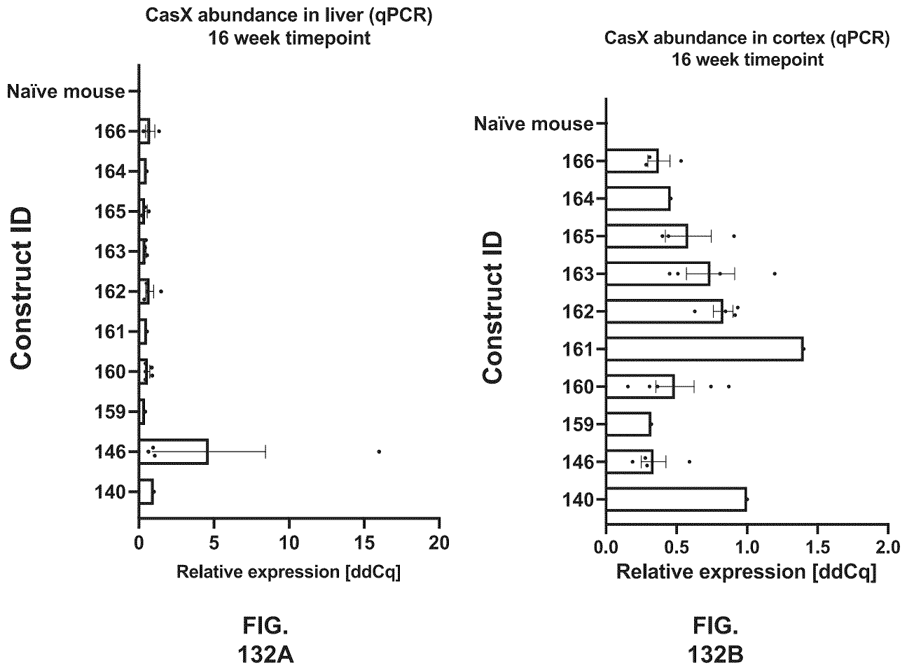

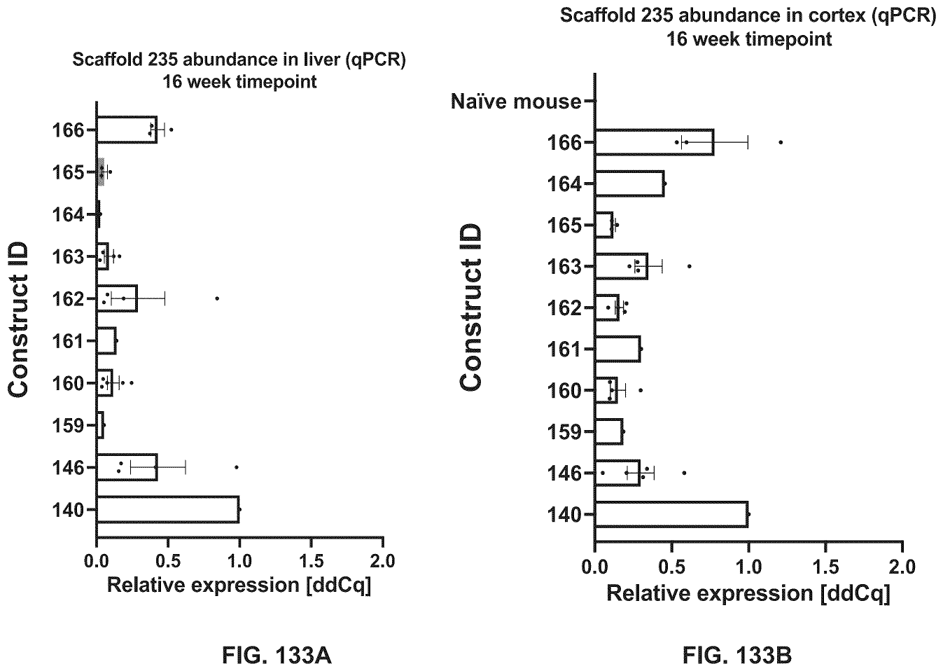

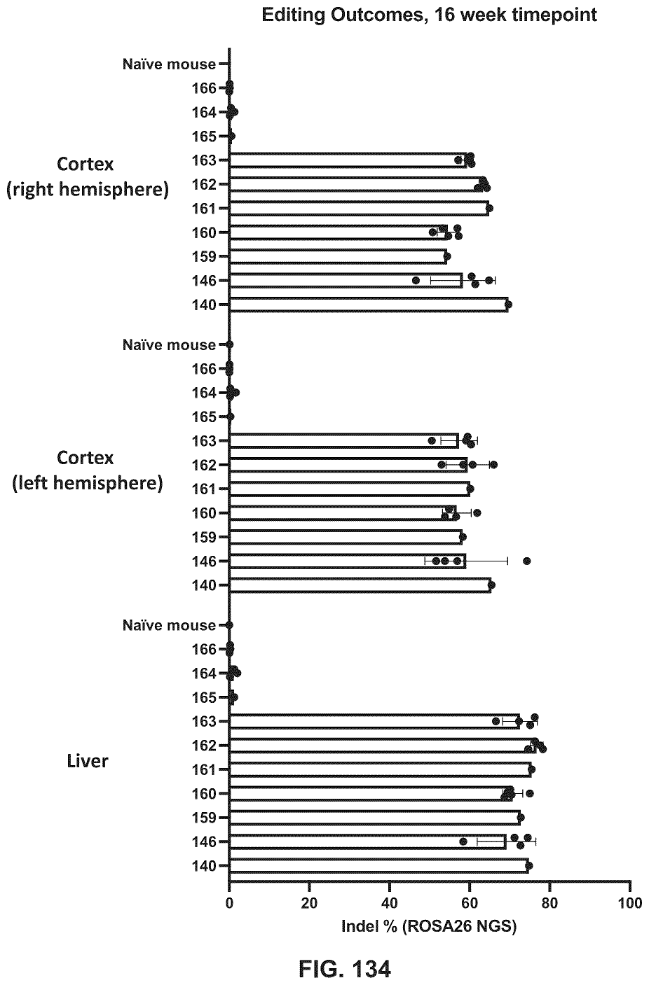

Three weeks following administration, the siAAV constructs achieved high levels of editing at the ROSA26 locus, which were only slightly lower than the construct targeting ROSA26 without a STALL site (construct 140; see FIG. 130). At 16 weeks, editing levels were generally higher than at three weeks (see FIG. 133A compared to FIG. 134). At the 16 week timepoint the siAAV constructs again showed a high level of editing compared to construct 140. The high level of editing observed indicates that editing in the liver was likely saturated.

At the 16 week timepoint, CasX mRNA expression in the cortex was decreased in the single STALL constructs (siAAV constructs 146, 159, and 160) compared to construct 140 (FIG. 132B). There was no decrease in CasX mRNA expression with the double STALL constructs in the cortex (FIG. 132B). In the liver, there was no differences in CasX mRNA expression between the constructs with or without STALL sites, with the exception the elevated expression seen in a mouse with construct 146 (FIG. 132A).

Significant decreases in scaffold 235 expression were observed in the cortex with all single STALL siAAVs, as well as the CTCN and GTCN double STALL constructs (siAAV constructs 162 and 163, respectively) compared to construct 140 (see FIG. 133B). Construct 165, with mismatched double STALL sites, was also significantly decreased. Specifically, the level of scaffold 235 abundance in mice administered constructs 146, 159, and 163 was significantly different from that in mice administered construct 140 at p<0.05. The level of scaffold 235 abundance in mice administered constructs 160, 162, and 165 was significantly different from that in mice administered construct 140 at p<0.01. There were no significant differences in scaffold 235 expression in the liver (FIG. 133B).

The results of this experiment demonstrate that siAAV constructs achieve gene editing in mice in vivo. This was true for siAAV constructs with one or two STALL sites, and with ATCN, CTCN, or GTCN STALL sites. Further, depletion of the guide scaffold was seen 16 weeks following in vivo administration for both siAAV constructs with single and double STALL sites. It is believed that variability between injections to mice may account for some of the variability seen in this experiment.

### Example 36: Systemic Delivery of AAV and siAAV in Mice

An experiment will be performed in which AAV and siAAV are administered systemically to mice, and genome editing and depletion of the siAAV vector will be assessed.

### Materials and Methods

AAVs and siAAVs encoding CasX variant 491 with guide scaffold 235 and a spacer targeting the ROSA26 locus were used. The AAV construct was construct 140, which has an sgRNA targeting ROSA26, and does not have a STALL site, as described in Example 35. The siAAV construct was construct 146, which has an sgRNA targeting ROSA26 and a single ATCN STALL site, as described in Example 35.

Mice were divided into five cohorts, with four mice per cohort. In the first four cohorts, mice were administered either 3e11 or 3e10 vg doses of either AAV or siAAV. In the fifth cohort, mice were administered a 3e11 vg dose with a 1:1 ratio of AAV and siAAV. This pooled dose of both XAAV and siAAV is believed to be an important control to account for the possibility of variability between injections to mice.

The AAV and siAAV vectors were administered by intraosseous infusion. Mice will be sacrificed at four timepoints, 3, 7, 28, and 60 days following AAV administration.

Liver and heart tissues will be harvested, and RNA, gDNA, and protein will be extracted. Target amplicons will be amplified from extracted gDNA with a set of primers targeting the mouse ROSA26 locus and processed for NGS as described in Example 23. The abundance of mRNA encoding CasX and sgRNA will be measured by RT-PCR, as described in Example 35. The abundance of CasX protein will be measured by western blot.

### Results:

The results are expected to show the rate of clearance of siAAV episome, the number of days required to reach undetectable levels of siAAV viral genome, the extent of CasX inactivation, and the editing rate of the AAV and siAAV vectors.

### Example 37: Demonstration that Varying the Placement and Orientation of the gRNA Promoter in the CasX:Dual-gRNA System Expressed from an All-In-One AAV Vector can Affect Editing of the Target Locus

The experiments in Example 9 showed that the CasX:dual-gRNA system packaged and delivered within a single AAV was able to edit the target gene. Here, experiments were performed to demonstrate that placement and orientation of the gRNA promoters within the AAV transgene to drive expression of dual gRNAs can affect the efficiency of the dual-cut editing of a target locus. Within the AAV plasmid, gRNA promoters could be placed upstream, downstream, or flanking the CasX construct and could be in a forward or reverse orientation. The various configurations of the dual-gRNA transcriptional units relative to the CasX construct within the AAV transgene are illustrated in FIGS. 35-36 and FIG. 112.

### Materials and Methods

AAV plasmid constructs were generated and cloned into a pAAV plasmid flanked with AAV2 ITRs using standard molecular cloning methods as described in Example 1. Briefly, dual-gRNA AAV plasmids were generated to express CasX variant 491 driven by the ubiquitous UbC promoter and two gRNA transcriptional units that each expressed a Pol III U6 promoter-guide scaffold 235-a specific spacer combination (spacer 12.7 targeting the tdTomato locus (SEQ ID NO: CTGCATTCTAGTTGTGGTTT, SEQ ID NO: 462) and/or a non-targeting spacer. In this example, the two gRNA transcriptional units were cloned relative to the CasX construct using configuration #1, #2, and #4 (illustrated in FIGS. 35-36) and tested. Table 62 below shows the combinations of spacers tested for each of the three configurations of dual gRNA units relative to the CasX construct.

TABLE 62
Combinations of a tdTomato-targeting spacer (12.7) and a non-targeting
(NT) spacer tested in this example in configuration #1, #2, and
#4 (illustrated in FIGS. 35-36) of dual gRNA units relative to
the CasX 491 construct. The “R” preceding the spacer denotes
the reverse orientation of the transcription of the indicated gRNA unit.

| Configuration # | Specific spacer combination tested |
|---|---|
| #1 | NT-CasX 491-NT |
| #1 | 12.7-CasX 491-NT |
| #1 | NT-CasX 491-12.7 |
| #1 | 12.7-CasX 491-12.7 |
| #4 | R.NT-CasX491-NT |
| #4 | R.12.7-CasX 491-NT |
| #4 | R.NT- CasX 491-12.7 |
| #4 | R.12.7- CasX 491-12.7 |
| #2 | CasX 491-NT-NT |
| #2 | CasX 491-12.7-NT |
| #2 | CasX 491-NT-12.7 |
| #2 | CasX-12.7-12.7 |

AAV nucleofection of tdTomato mNPCs was performed as described in Example 1. Briefly, 125 ng of AAV plasmid encoding for XAAVs expressing the CasX:dual-gRNA system with the various configurations listed in Table 62 were nucleofected mNPCs. Five days post-nucleofection, mNPCs were harvested for editing analysis at the tdTomato locus by FACS, as described in Example 1. For comparison, AAV plasmid encoding for XAAVs expressing CasX 491 with a single gRNA transcriptional unit using spacer 12.7 was also used in this example.

AAV production and AAV titering were performed as described in Example 1.

AAV Transduction of tdTomato mNPCs, Followed by Flow Cytometry:

˜10,000 mNPCs were seeded per well in PLF-coated 96-well plates; 48 hours later, seeded cells were transduced with AAVs expressing the CasX:dual-gRNA system of various configurations (Table 62). All viral infection conditions were performed in triplicate, with a normalized number of viral genomes (cg) among experimental vectors, in a series of three-fold dilution of MOI ranging from −1E5 to 1E3 vg/cell. Five days post-transduction, XAAV-treated mNPCs were harvested for editing analysis at the tdTomato locus by FACS, as described earlier in Example 1. For comparison, AAVs expressing CasX 491 with a single gRNA transcriptional unit using spacer 12.7 were also assayed in this example.

### Results:

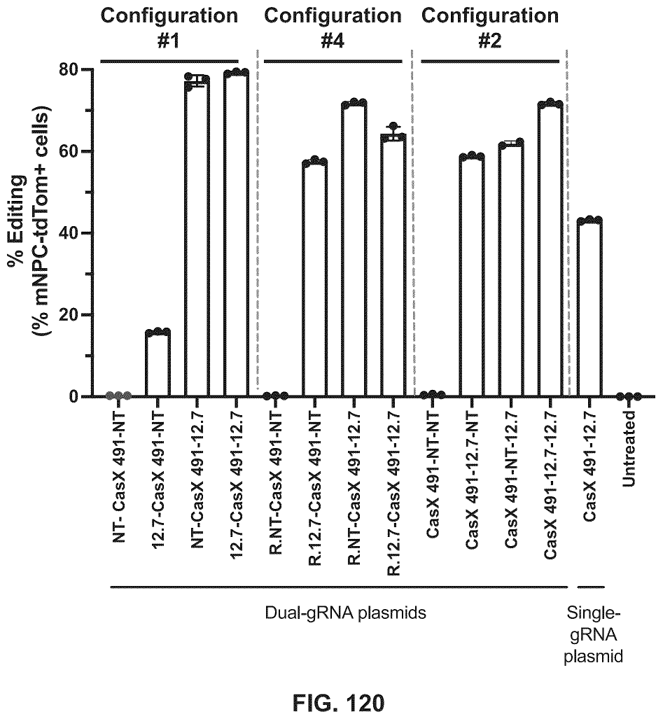

tdTomato mNPCs were nucleofected with AAV plasmids encoding for dual-guide AAVs expressing the CasX:dual-gRNA system in various vector configurations with different spacer combinations of spacer 12.7 or a non-targeting spacer (listed in Table 62). Editing levels at the tdTomato locus were subsequently assessed to determine the difference in editing level achieved and driven by a spacer in a particular orientation and position, and the results are illustrated in FIG. 120. The data indicate that for configuration #1 (FIG. 35), the editing levels achieved appeared to be primarily driven by the second gRNA positioned on the 3′ end of the CasX construct in the sense orientation, since use of the NT-CasX 491-12.7 AAV construct resulted in ˜80% editing, while use of the 12.7-CasX 491-NT construct resulted in ˜20% editing (FIG. 120). In addition, use of the NT-CasX 491-12.7 construct resulted in similar levels of editing as use of the AAV construct with two 12.7 spacers (12.7-CasX 491-12.7; FIG. 120). The data further showed that positioning and orienting the gRNA units in configurations #4 and #2 (FIG. 35) appeared to induce similar levels of editing overall between the two gRNA units within an AAV transgene (FIG. 120).

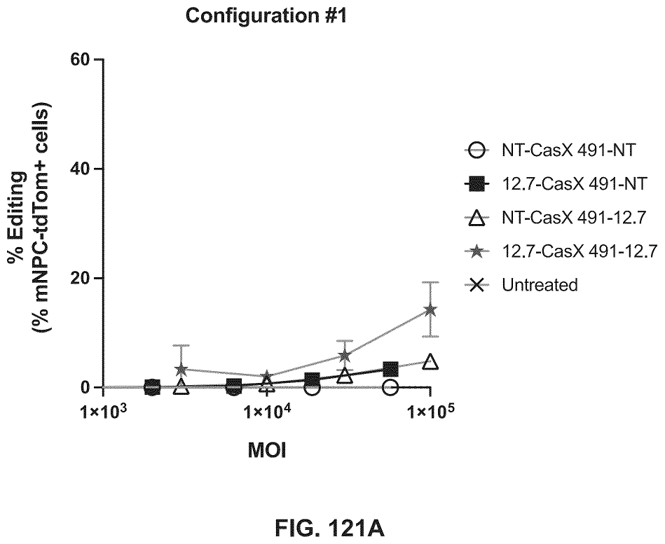

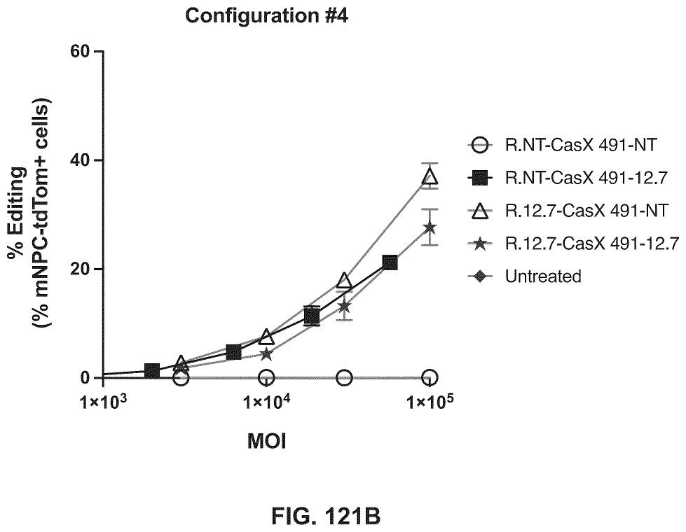

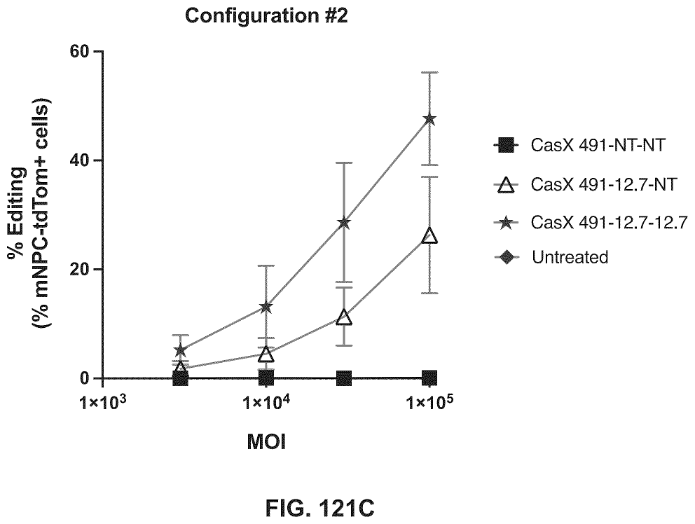

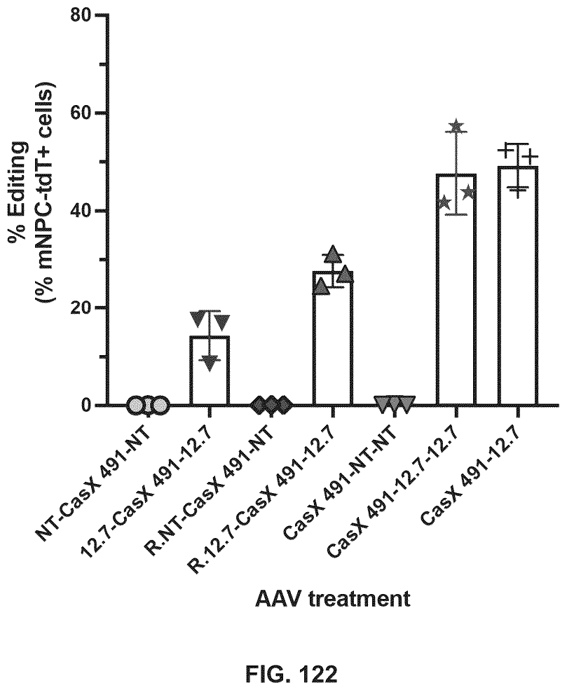

tdTomato mNPCs were also transduced with dual-guide AAVs expressing the CasX:dual-gRNA system in configurations #1, #2, and #4 (FIGS. 35-36) with different spacer combinations (Table 62) at varying MOIs, and editing levels were subsequently assessed, with the resulted portrayed in FIGS. 121A-121C. The data demonstrate that for configuration #1, each gRNA unit was able to achieve similar levels of editing, when comparing the editing levels induced by AAVs containing the 12.7-CasX 491-NT construct with those achieved by AAVs containing the NT-CasX 491-12.7 construct (FIG. 121A). Notably, AAVs containing the 12.7-CasX 491-12.7 construct induced comparatively higher editing than either of the spacer 12.7-NT combination (FIG. 121A). For configuration #4, AAVs containing the R.12.7-CasX 491-NT construct appeared to achieve a slightly higher editing level at the highest MOI tested (˜-1E5 vg/cell), suggesting that the gRNA placed 5′ of the CasX construct and in antisense orientation was more active than its counterpart positioned 3′ to the CasX construct in the sense orientation (FIG. 121B). Interestingly, AAVs containing the R12.7-CasX 491-12.7 construct did not induce the highest level of editing out of all the spacer combinations tested for configuration #4, suggesting a saturation in editing levels achieved given the limitation in gRNA expression (FIG. 121B). Lastly, for configuration #2, the data demonstrate that at the highest MOI tested of 1E5, XAAVs with the CasX 491-12.7-NT construct induced ˜20% editing and XAAVs with the CasX 491-12.7-12.7 induced ˜40% editing, suggesting that each gRNA in either position in the sense orientation was able to drive a similar level of editing as its partner (FIG. 121C). A comparison of AAVs expressing a CasX:dual-guide system with two 12.7 spacers in configuration #1, #2, and #4 revealed that use of AAVs expressing the CasX:dual-gRNA system in configuration #2 induced the highest level of editing (˜47.6±8%), compared to ˜27.7±3.3% editing and ˜14.3±5% editing for configuration #4 and #1 respectively (FIG. 122).

The results from these experiments demonstrate that use of gRNAs in different positions and orientations relative to the CasX encoding construct within the AAV transgene can achieve efficient dual-cut editing at the target locus. Furthermore, varying the specific position and orientation of the gRNA unit can affect the editing efficiency. The findings from these experiments indicate the potential to use the dual-guide system within the context of siAAV vectors. Using the dual-guide system would enable effective modulation and tuning of CasX-mediated editing and self-cleavage activity that would progressively remove the AAV episome in edited cells to mitigate potential off-target effects. More specifically, siAAV vectors could be designed such that the therapeutic targeting gRNA would be in the position and orientation that would induce highly effective editing, and a second gRNA within the same AAV transgene would be in the position and orientation that would enable its expression at lower but sufficient levels to induce self-inactivation of CasX.

TABLE 63
Encoding DNA sequences for CasX variants

| CasX | DNA SEQ ID NO |
|---|---|
| CasX 438 | 747 |
| CasX 488 | 748 |
| CasX 491 | 749 |
| CasX 515 | 750 |
| CasX 527 | 751 |
| CasX 535 | 752 |
| CasX 536 | 753 |
| CasX 537 | 754 |
| CasX 583 | 755 |
| CasX 660 | 756 |
| CasX 661 | 757 |
| CasX 662 | 758 |
| CasX 663 | 759 |
| CasX 664 | 760 |
| CasX 668 | 761 |

Table 64 provides all the individual components of the constructs from Examples 1-37.

TABLE 64
AAV construct component sequences for Examples 1-37

| | | | SEQ ID |
|---|---|---|---|
| Component | Name | AAV construct ID | NO: |
| 5′ ITR | AAV2 ITR (alternate) | 1-174, 177-186, 207-220 | 423 |
| | CpG-depleted 5′ ITR | — | 2918 |
| Enhancer + | CMV | 1-3, 7, 24-33, 44-52, 103-117, 211-214 | 463 |
| core promoter | N/A | 1-3, 7, 24-33, 44-52, 64-71, 103-117, 156 | 493 |
| | Syn 1 | 65 | 764 |
| | NPC5 | 66 | 765 |
| | NPC7 | 67 | 766 |
| | NPC127 | 68 | 767 |
| | NPC190 | 69 | 768 |
| | NPC249 | 70 | 769 |
| | NPC286 | 71 | 770 |
| Enhancer | Muscle Enhancer 1 | — | 4057 |
| | Muscle Enhancer 2 | — | 4058 |
| | Muscle Enhancer 3 | — | 4059 |
| | Muscle Enhancer 4 | — | 4060 |
| | Muscle Enhancer 5 | — | 4061 |
| | Muscle Enhancer 6 | — | 4062 |
| | Muscle Enhancer 7 | — | 4063 |
| | Muscle Enhancer 8 | — | 4064 |
| | Muscle Enhancer 9 | — | 4065 |
| | Muscle Enhancer 10 | — | 4066 |
| | Muscle Enhancer 11 | — | 4067 |
| | Muscle Enhancer 12 | — | 4068 |
| | MyoD Enhancer | — | 4069 |
| | Cardiac Muscle Enhancer 1 | — | 4070 |
| | Cardiac Muscle Enhancer 2 | — | 4071 |
| | Cardiac Muscle Enhancer 3 | — | 4072 |
| | Cardiac Muscle Enhancer 4 | — | 4073 |
| | Myoblast Muscle Enhancer 1 | — | 4074 |
| | Myoblast Muscle Enhancer 2 | — | 4075 |
| | Myoblast Muscle Enhancer 3 | — | 4076 |
| | Myoblast Muscle Enhancer 4 | — | 4077 |
| | Myoblast Muscle Enhancer 5 | — | 4078 |
| | Skeletal Muscle Enhancer 1 | — | 4079 |
| | Skeletal Muscle Enhancer 2 | — | 4080 |
| | Skeletal Muscle Enhancer 3 | — | 4081 |
| | Skeletal Muscle Enhancer 4 | — | 4082 |
| | Skeletal Muscle Enhancer 5 | — | 4083 |
| | Skeletal Muscle Enhancer 6 | — | 4084 |
| | Skeletal Muscle Enhancer 7 | — | 4085 |
| | Skeletal Muscle Enhancer 8 | — | 4086 |
| | Skeletal Muscle Enhancer 9 | — | 4087 |
| Protein | CMV | 1-3, 7, 24-33, 44-52, 103-117, 211-214 | 463 |
| promoter | UbC | 4, 34-37, 53, 78, 79-102, 119-155, 207-210, | 464 |
| | | 215, 220 | |
| | EFS | 5, 38-40 | 465 |
| | CMV-s | 6, 41-43 | 466 |
| | CMVd1 | 8 | 467 |
| | CMVd2 | 9 | 468 |
| | miniCMV | 10 | 469 |
| | HSVTK | 11 | 470 |
| | miniTK | 12 | 471 |
| | miniIL2 | 13 | 472 |
| | GRP94 | 14 | 473 |
| | Supercore 1 | 15 | 474 |
| | Supercore 2 | 16 | 475 |
| | Supercore 3 | 17 | 476 |
| | Mecp2 | 18 | 477 |
| | CMVmini | 19 | 478 |
| | CMVmini2 | 20 | 479 |
| | miniCMVIE | 21 | 480 |
| | adML | 22 | 481 |
| | hepB | 23 | 482 |
| | RSV | 54 | 483 |
| | hSyn | 55 | 484 |
| | SV40 | 56 | 485 |
| | hPGK | 57 | 486 |
| | Jet | 58, 72-74 | 487 |
| | Jet + UsP intron | 59, 75-77 | 488 |
| | hRLP30 | 60 | 489 |
| | hRPS18 | 61 | 490 |
| | CBA | 62 | 491 |
| | CBH | 63 | 492 |
| | CMV core | 64 | 493 |
| | U1a | 177, 180, 181, 182 | 2930 |
| | CpG-reduced U1a | 178, 206 | 2907 |
| | CpG-depleted U1a | 179, 205 | 2908 |
| | CpG-reduced U6 | 180 | 2909 |
| | CpG-depleted U6 | 181, 205, 206 | 2910 |
| | CpG-reduced UbC | 184 | 2904 |
| | Strongly CpG-reduced UbC | 185 | 2905 |
| | CpG-depleted UbC | 186 | 2906 |
| | SFCp | — | 2931 |
| | miniSV40 | — | 2932 |
| | pJB42CAT5 | — | 2933 |
| | MLP | — | 2934 |
| | miniEF1α | — | 2935 |
| | hRPL13a | — | 2936 |
| 5′ NLS aa | 1X SV40 NLS | — | 771 |
| sequence | 4X SV40 NLS | 121-123 | 772 |
| | 1X Cmyc NLS | 83, 84, 89-102, 124-131, 135-137, 141-155, | 541 |
| | | 157-174, 177-186, 207-210, 215-220 | |
| | 2X Cmyc NLS | 127-129 | 542 |
| | 4X Cmyc NLS | 130, 131 | 543 |
| | 6X Cmyc NLS | 135-137, 142 | 544 |
| | 1X Nucleoplasmin NLS | 132-134 | 545 |
| | 2X Nucleoplasmin NLS | 138-140 | 546 |
| | 1X Cmyc 1X SV40 NLS | — | 547 |
| | 1X Cmyc 2′ 1X SV40 NLS | — | 548 |
| | 1X Cmyc 2′ NLS | — | 549 |
| | 3X Cmyc 2′ NLS | — | 550 |
| | 4X Cmyc 2′ NLS | — | 551 |
| | 1X CPV NLS 1N | — | 552 |
| | 2X CPV NLS IN | — | 553 |
| | 1X hBOVc NLS 1N | — | 554 |
| | 1X hBOVc NLS 2N | — | 555 |
| | 1X SIRT NLS | — | 556 |
| | 2X SIRT NLS | — | 557 |
| | 1X Cmyc NLS 1X BPSV40 | — | 558 |
| | NLS GGS | | |
| | 1X Cmyc NLS 1X BPSV40 | — | 559 |
| | NLS PPPPG | | |
| | 1X Cmyc NLS 1X BPSV40 | — | 560 |
| | NLS px330 PG | | |
| | 1X Cmyc NLS 1X BPSV40 | — | 561 |
| | NLS (GGGS)2 PG | | |
| | 1X Cmyc NLS 1X BPSV40 | — | 562 |
| | NLS P(GGGS)2 PG | | |
| | 1X Cmyc NLS 1X BPSV40 | — | 563 |
| | NLS alpha PG | | |
| | 1X Cmyc NLS 1X BPSV40 | — | 564 |
| | NLS PG | | |
| | 1X Cmyc GGS 1X SV40 | — | 565 |
| | GGS | | |
| | 1X Cmyc PPP 1X SV40 PG | — | 566 |
| | 1X Cmyc PG | — | 567 |
| | 1X Cmyc (GGGS)3 | — | 568 |
| | 1X Cmyc PPP | — | 569 |
| | 1X Cmyc (GGGS)3 PPP | — | 570 |
| | 1X SV40 PPP | — | 571 |
| | 1X SV40 GGS | — | 572 |
| CasX | CpG-depleted cMycNLS- | 205, 206 | 2916 |
| | Stx491-cMycNLS | | |
| 3′ NLS aa | 1X SV40 NLS | — | 573 |
| sequence | 4X SV40 NLS | 149 | 574 |
| | 6S SV40 NLS | — | 575 |
| | 1X Cmyc NLS | 141, 142, 150, 157-174, 177-186, 207-210 | 576 |
| | 2X Cmyc NLS | 151 | 577 |
| | 4x Cmyc NLS | — | 578 |
| | 6x Cmyc NLS | 152 | 579 |
| | 1X Nucleoplasmin NLS | 119, 122, 125, 128, 130, 133, 136, 139, 153 | 580 |
| | 2X Nucleoplasmin NLS | 120, 123, 126, 129, 131, 134, 137, 140, 154 | 581 |
| | 2x Nucleoplasmin 2x SV40 | 155 | 582 |
| | NLS | | |
| | B19 NLS 1C | — | 583 |
| | BoV NLS 3C | — | 584 |
| | 1X SV40 GS 1X | — | 585 |
| | Nuceloplasmin NLS | | |
| | GP vBPSV40 12aa SV40 | 143 | 586 |
| | NLS | | |
| | (GGGs)2vBPSV40 12aa | — | 587 |
| | SV40 | | |
| | 3′alphahelix vBPSV40 12aa | 144 | 588 |
| | SV40 | | |
| | GP SV40 GGS vBPSV40 | — | 589 |
| | 12aa SV40 | | |
| | GP alpha helix Cmyc NLS | 145 | 590 |
| | GP (GGGS)3 Cmyc NLS | 146 | 591 |
| | GP SV40 PPP Cmyc NLS | 148 | 592 |
| | GP Cmyc NLS | 147 | 593 |
| | TGGGPGGGAAAGSGS- | — | 597 |
| | 1xSV40-GS-Nuc | | |
| | TGGGPGGGAAAGSGS- | — | 599 |
| | 1×SV40-GS | | |
| | PPPlinker 1xSV40 PPPlinker | — | 600 |
| | GGSlinker 1xSV40 | — | 601 |
| | PPPlinker | | |
| | PPPlinker 1xSV40 | — | 602 |
| | GGSlinker 1xSV40 | — | 603 |
| | GGSlinker 1xSV40 | — | 604 |
| | (GGS)3linker | | |
| | GGSlinker 2xSV40 | — | 605 |
| | (GGS)3linker 1xSV40 GGS | — | 606 |
| | 1XSV40 | | |
| | PPP(GGGS)3linker 1xCmyc | — | 609 |
| | PPPlinker 1xCmyc | — | 608 |
| | PPP(GGGS)3linker 1xCmyc | — | 773 |
| PTRE | WPRE1 | 35, 38, 41, 72, 75, 78, 81, 83 | 524 |
| | WPRE2 | 36, 39, 42, 73, 76, 79, 82, 84 | 525 |
| | WPRE3 | 34, 37, 40, 43, 74, 77, 80 | 526 |
| PolyA signal | bGH | 1-23, 32, 33, 35-174, 177-181, 183-186, | 514 |
| | | 207-220 | |
| | hGH | 24 | 515 |
| | hGHshort | 25 | 516 |
| | HSVTK | 26 | 517 |
| | SynPolyA | 27 | 518 |
| | SV40 | 28 | 519 |
| | SV40short | 29 | 520 |
| | bglob | 30 | 521 |
| | bglobshort | 31 | 522 |
| | SV40polyA late | 34 | 523 |
| | CpG-depleted bGH | 182, 205, 206 | 2917 |
| | T7 Tphi | 222 | 3997 |
| | CaMV | 223 | 3998 |
| | RDH1 | 224 | 3999 |
| | Sv40 polyA late | 225 | 4000 |
| RNA | human U6 | 1-31, 34-84, 103-157, 177-179, 182-186, | 494 |
| promoter | | 207, 209, 211-220 | |
| | Human U6 (reverse | 208, 210 | 4001 |
| | complement) | | |
| | H1 | 32, 158 | 495 |
| | 7SK | 33 | 496 |
| | hU6 variant 1 | 85, 89 | 497 |
| | hU6 variant 2 | 86 | 498 |
| | hU6 variant 3 | 87 | 499 |
| | hU6 variant 4 | 88 | 500 |
| | hU6 variant 5 | 90 | 501 |
| | hU6 variant 6 | 91 | 502 |
| | hU6 variant 7 | 92 | 503 |
| | hU6 variant 8 | 93 | 504 |
| | hU6 variant 9 | 94 | 505 |
| | hU6 variant 10 | 95 | 506 |
| | hU6 variant 11 | 96 | 507 |
| | hU6 variant 12 | 97 | 508 |
| | hU6 variant 13 | 98 | 509 |
| | hU6 variant 14 | 99 | 510 |
| | hU6 variant 15 | 100 | 511 |
| | hU6 variant 16 | 101 | 512 |
| | hU6 variant 17 | 102 | 513 |
| | H1 core | 159 | 2688 |
| | H1 core + 7SK hybrid 1 | 160 | 2689 |
| | H1 core + 7SK hybrid 2 | 161 | 2690 |
| | H1 core + 7SK hybrid 3 | 162 | 2691 |
| | H1 core + 7SK hybrid 4 | 163 | 2692 |
| | H1 core + 7SK hybrid 5 | 164 | 2693 |
| | H1 core + 7SK hybrid 6 | 165 | 2694 |
| | H1 core + 7SK hybrid 7 | 166 | 2695 |
| | H1 core + 7SK hybrid 8 | 167 | 2696 |
| | H1 core + 7SK hybrid 9 | 168 | 2697 |
| | H1 core + U6 hybrid 1 | 169 | 2698 |
| | H1 core + U6 hybrid 2 | 170 | 2699 |
| | H1 core + 7SK + U6 hybrid 1 | 171 | 2700 |
| | H1 core + U6 hybrid 3 | 172 | 2701 |
| | H1 core + 7SK + U6 hybrid 2 | 173 | 2702 |
| | H1 core + 7SK + U6 hybrid 3 | 174 | 2703 |
| | hU6 isoform 2 | — | 2704 |
| | hU6 isoform 3 | — | 2705 |
| | hU6 isoform 4 | — | 2706 |
| | hU6 isoform 5 | — | 2707 |
| | hU6-CpG reduced | 180 | 2909 |
| | hU6-CpG reduced | 181 | 2910 |
| | mU6 | — | 2708 |
| | CpG-reduced | — | 2911 |
| | hU6 Isoform 2 | | |
| | CpG-depleted hU6 Isoform 2 | — | 2912 |
| | CpG-depleted hU6 Isoform 3 | — | 2913 |
| | CpG-depleted hU6 Isoform 4 | — | 2914 |
| | CpG-depleted hU6 Isoform 5 | — | 2915 |
| 3′ ITR | AAV2 ITR | 1-174, 177-186 | 424 |
| | CpG-depleted 3′ ITR | — | 2919 |

The first rows of Table 65 provide sequences of shRNAs 1-12. These shRNA sequences were incorporated at the 5′ end of the siAAV transgene that was used to transfect the packaging cells (Examples 17 & 18). The shRNA constructs are labeled 1-12, and 29 in the second column. The following rows list the transgene components in the order in which they are arranged in the transgene. Constructs 17 to 23 have the shRNA in separate plasmids. These are not appended to the siAAV transgene but were transfected as separate plasmids in the packaging cell line. STALL 24-28 are constructs that have the self-limiting segments.

TABLE 65
shRNA and siAAV Constructs

| | SiAAV | | SEQ |
|---|---|---|---|
| Category | construct ID | Component | ID NO: |
| EFGP 3′ | 1 | EF1a-EGFP-shRNA1a | 2937 |
| UTR | 2 | EF1a-EGFP-shRNA2a | 2938 |
| shRNA | 3 | EF1a-EGFP-shRNA3a | 2939 |
| Backbones | 4 | EF1a-EGFP-shRNA4a | 2940 |
| | 5 | EF1a-EGFP-shRNA5a | 2941 |
| | 6 | EF1a-EGFP-shRNA6a | 2942 |
| | 7 | EF1a-EGFP-shRNA7a | 2943 |
| | 8 | EF1a-EGFP-shRNA8a | 2944 |
| | 9 | EF1a-EGFP-shRNA9a | 2945 |
| | 10 | EF1a-EGFP-shRNA10a | 2946 |
| | 11 | EF1a-EGFP-shRNA11a | 2947 |
| | 12 | EF1a-EGFP-shRNA12a | 2948 |
| | 13 | BB59.shRNA8.shRNA8 | 2949 |
| | 14 | BB59.shRNA8.shRNA11 | 798 |
| | 15 | BB59.shRNA8.shRNA12 | 799 |
| | 16 | BB59.shRNA11.shRNA12 | 800 |
| | 29 | EF1a-EGFP-DEST | 2950 |
| | 89, 90, 141 | U6-shRNA8a | 2951 |
| U6 + | 87, 88, 142 | U6-shRNA8a Scramble | 2952 |
| shRNA8 | 77, 78 | U6-shRNA8b | 2953 |
| Scaffold | 75, 76 | U6-shRNA8b Scramble | 2954 |
| Variation | 85, 86 | U6-shRNA8c | 2955 |
| Silencing | 83, 84 | U6-shRNA8c Scramble | 2956 |
| Backbones | 81, 82 | U6-shRNA8d | 2957 |
| | 79, 80 | U6-shRNA8d Scramble | 2958 |
| Stacked | 91, 104 | U6-shRNA8a 7SK shRNA8b | 2959 |
| shRNA | 92, 105 | U6-shRNA8a H1 shRNA8b | 2960 |
| Backbones | 93, 106, 143 | U6-shRNA8a 7SK shRNA8c | 2961 |
| | 94, 107, 144 | U6-shRNA8a H1 shRNA8c | 2962 |
| | 95, 108 | U6-shRNA8a 7SK-shRNA8b | 2963 |
| | | H1 shRNA8c | |
| | 96, 109 | U6-shRNA8a 7SK-shRNA8cH1 | 2964 |
| | | shRNA8b | |
| | 97, 110 | mU6-shRNA8a | 2965 |
| | 98, 111 | mU6-shRNA8a 7SK shRNA8b | 2966 |
| | 99, 112 | mU6-shRNA8a H1 shRNA8b | 2967 |
| | 100, 113 | mU6-shRNA8a 7SK shRNA8c | 2968 |
| | 101, 114 | mU6-shRNA8a H1 shRNA8c | 2969 |
| | 102, 115 | mU6-shRNA8a | 2970 |
| | | 7SK shRNA8b H1 shRNA8c | |
| | 103, 116 | mU6-shRNA8a | 2971 |
| | | 7SK shRNA8c H1 shRNA8b | |
| iRNA and | 122 | U6-iRNA1 | 2972 |
| dgRNA | 123 | U6-iRNA2 | 2973 |
| Backbones | 124 | U6-iRNA3 | 2974 |
| | 125 | U6-iRNA4 | 2975 |
| | 130 | U6-174NT | 2976 |
| | 131 | U6-174NS | 2977 |
| | 132 | U6-234NT | 2978 |
| | 133 | U6-234NS | 2979 |
| | 134 | U6-235NT | 2980 |
| | 135 | U6-235NS | 2981 |

TABLE 66
Sequences of AAV transgenes and standalone plasmids

| Category | Construct ID | Component | SEQ ID NO |
|---|---|---|---|
| AAV | 1-12, 29 | 5′ ITR | 423 |
| transgenes | | Buffer seq | 788 |
| * and | | CMV Enhancer + Promoter | 527 |
| standalone | | Buffer seq | 789 |
| Plasmids | | Kozak | NA |
| | | Start codon | NA |
| | | NLS | 790 |
| | | linker | NA |
| | | CasX 491 Protein | 791 |
| | | linker | NA |
| | | NLS | 792 |
| | | HA tag | 793 |
| | | linker + stop | NA |
| | | Buffer seq | 794 |
| | | poly A | 514 |
| | | Buffer seq | NA |
| | | U6 promoter | 494 |
| | | buffer seq | NA |
| | | Scaffold | 691 |
| | | Spacer | 795 |
| | | Buffer seq | 796 |
| | | 3′ ITR | 424 |
| shRNA | 13 | BB59.shRNA8.shRNA8 | 797 |
| backbones | | | |
| shRNA | 17 | EF1a.eGFP.shRNA8 | 801 |
| standalone | 18 | EF1a.eGFP.shRNA11 | 802 |
| plasmids | 19 | EF1a.eGFP.shRNA12 | 803 |
| | 20 | EF1a.eGFP.shRNA8.shRNA8 | 804 |
| | 21 | EF1a.eGFP.shRNA8.shRNA11 | 805 |
| | 22 | EF1a.eGFP.shRNA8.shRNA12 | 806 |
| | 23 | EF1a.eGFP.shRNA11.shRNA12 | 807 |
| STALLs | 24-28 | 5′ ITR | 423 |
| | | Protein Promoter | 527 |
| | 24 | STALL-TTC | 808 |
| | 25 | STALL-CTC | 809 |
| | 26 | STALL-ATC | 810 |
| | 27 | STALL-GTC | 811 |
| | 28 | STALL-GGGN | 812 |
| | 24-28 | Protein | 791 |
| | | PolyA Signal Sequence | 514 |
| | | RNA Promoter | 494 |
| | | Scaffold | 691 |
| | | Spacer | 462 |
| | | 3′ ITR | 424 |
| Scaffolds | 31-47 | 5′ITR | 423 |
| | | buffer seq | 788 |
| | | enhancer | 813 |
| | | promoter | 493 |
| | | buffer seq | 814 |
| | | kozak | 829 |
| | | start codon | NA |
| | | 5′NLS | 790 |
| | | 5′linker | NA |
| | | CasX 491 Protein | 791 |
| | | 3′NLS linker | NA |
| | | 3′NLS | 792 |
| | | tag | 793 |
| | | linker | NA |
| | | buffer seq | 794 |
| | | polyA | 514 |
| | | polIII prom | 494 |
| | | buffer | NA |
| | 31 | Scaffold 174 | 691 |
| | 34 | Scaffold 221 | 815 |
| | 35 | Scaffold 222 | 816 |
| | 36 | Scaffold 223 | 817 |
| | 37 | Scaffold 224 | 818 |
| | 38 | Scaffold 225 | 819 |
| | 39 | Scaffold 229 | 820 |
| | 40 | Scaffold 230 | 821 |
| | 41 | Scaffold 231 | 822 |
| | 42 | Scaffold 232 | 823 |
| | 43 | Scaffold 233 | 824 |
| | 44 | Scaffold 234 | 825 |
| | 45 | Scaffold 235 | 698 |
| | 46 | Scaffold 236 | 827 |
| | 47 | Scaffold 237 | 828 |
| | 31-47 | Spacer | 462 |
| | | Buffer | 796 |
| | | 3′ITR | 424 |
| Double | 48-52 | 5′ ITR | 423 |
| guides | | Protein Promoter | 527 |
| | | Protein | 791 |
| | | PolyA Signal Sequence | 514 |
| | 48 | RNA Promoter 1 | 494 |
| | | RNA Promoter 2 | 494 |
| | | Scaffold 1 | 691 |
| | | Scaffold 2 | 691 |
| | | Spacer 1 | 537 |
| | | Spacer 2 | 829 |
| | 49 | RNA Promoter 1 | 494 |
| | | RNA Promoter 2 | 494 |
| | | Scaffold 1 | 691 |
| | | Scaffold2 | 691 |
| | | Spacer 1 | 829 |
| | | Spacer 2 | 830 |
| | 50 | RNA Promoter 1 | 494 |
| | | RNA Promoter 2 | 494 |
| | | Scaffold 1 | 691 |
| | | Scaffold 2 | 691 |
| | | Spacer 1 | 462 |
| | | Spacer 2 | 462 |
| | 51 | RNA Promoter 1 | 494 |
| | | RNA Promoter 2 | 494 |
| | | Scaffold 1 | 691 |
| | | Scaffold 2 | 691 |
| | | Spacer 1 | 462 |
| | | Spacer 2 | 536 |
| | 52 | RNA Promoter 1 | 494 |
| | | RNA Promoter 2 | 494 |
| | | Scaffold 1 | 69 |
| | | Scaffold 2 | 691 |
| | | Spacer 1 | 537 |
| | | Spacer 2 | 462 |
| | 48-52 | 3′ ITR | 424 |
| RNA | 54-73 | 5′ ITR | 423 |
| promoters | | buffer seq | 788 |
| | | promoter | 464 |
| | | stuffer + kozak | 831 |
| | | start codon | NA |
| | | 5′ NLS | 832 |
| | | 5′ linker | |
| | | CasX 491 Protein | 791 |
| | | 3′ NLS linker | NA |
| | | 3′ NLS | 792 |
| | | tag + stop codon | 833 |
| | | buffer seq | 794 |
| | | polyA | 514 |
| | | buffer seq | NA |
| | 54 | RNA promoter 1 | 497 |
| | 55 | RNA promoter 2 | 498 |
| | 56 | RNA promoter 3 | 499 |
| | 57 | RNA promoter 4 | 500 |
| | 58 | RNA promoter 5 | 497 |
| | 59 | RNA promoter 6 | 501 |
| | 60 | RNA promoter 7 | 502 |
| | 61 | RNA promoter 8 | 503 |
| | 62 | RNA promoter 9 | 504 |
| | 63 | RNA promoter 10 | 505 |
| | 64 | RNA promoter 11 | 506 |
| | 65 | RNA promoter 12 | 507 |
| | 66 | RNA promoter 13 | 508 |
| | 67 | RNA promoter 14 | 509 |
| | 68 | RNA promoter 15 | 510 |
| | 69 | RNA promoter 16 | 511 |
| | 70 | RNA promoter 17 | 512 |
| | 71 | RNA promoter 18 | 513 |
| | 72 | H1 | 495 |
| | 73 | 7SK | 496 |
| | 54-73 | buffer | NA |
| | | scaffold | 691 |
| | | spacer | 462 |
| | | buffer | 796 |
| | | 3′ ITR | 424 |
| Silencing | 73 | 5′ ITR | 423 |
| Backbones + | | buffer | 834 |
| Other | | Protein Promoter | 836 |
| Construct | | STALL + KOZAK | 837 |
| Transgenes | | start codon | NA |
| ONLY | | 5′ NLS | 838 |
| | | 5′ linker | NA |
| | | CasX 491 Protein | 791 |
| | | 3′ NLS linker | NA |
| | | 3′ NLS | 839 |
| | | stop codon | NA |
| | | STALL | 840 |
| | | PolyA Signal Sequence | 514 |
| | | buffer | NA |
| | | RNA Promoter | 494 |
| | | buffer | NA |
| | | Scaffold | 698 |
| | | Spacer | 841 |
| | | buffer | 796 |
| | | 3′ ITR | 424 |
| | 74 | 5′ ITR | 423 |
| | | buffer | 834 |
| | | Protein Promoter | 835 |
| | | buffer + kozak | 2982 |
| | | start codon | NA |
| | | 5′ NLS | 838 |
| | | 5′ linker | NA |
| | | CasX 535 Protein | 752 |
| | | 3′ NLS linker | NA |
| | | 3′ NLS | 839 |
| | | stop codon | NA |
| | | buffer | 794 |
| | | PolyA Signal Sequence | 514 |
| | | buffer | NA |
| | | RNA Promoter | 494 |
| | | buffer | NA |
| | | Scaffold | 698 |
| | | Spacer | 841 |
| | | buffer | 796 |
| | | 3′ ITR | 424 |
| | 30, | 5′ ITR | 423 |
| | 75, 77, 79, 81, 83, | buffer seq | 788 |
| | 85, 87, 89, 120, | promoter | 464 |
| | 126-127 | buffer seq | 789 |
| | | kozak | NA |
| | | start codon | NA |
| | | 5′ NLS | 790 |
| | | 5′ linker | NA |
| | | CasX 491 Protein | 791 |
| | | 3′ NLS linker | NA |
| | | 3′ NLS | 792 |
| | | tag | 793 |
| | | linker-stop codon | NA |
| | | buffer seq | 794 |
| | | polyA | 514 |
| | | buffer seq | NA |
| | | polIII prom | 494 |
| | | buffer | NA |
| | | scaffold | 691 |
| | | spacer | 462 |
| | | buffer | 796 |
| | | 3′ITR | 424 |
| | 32 | 5′ ITR | 423 |
| | | buffer seq | 788 |
| | | promoter | 464 |
| | | buffer seq + STALL site | 842 |
| | | kozak | NA |
| | | start codon | NA |
| | | 5′ NLS | 832 |
| | | 5′ linker | NA |
| | | CasX 491 Protein | 791 |
| | | 3′ NLS linker | NA |
| | | 3′ NLS | 792 |
| | | tag | 793 |
| | | linker-stop codon | NA |
| | | buffer seq + STALL site | 843 |
| | | polyA | 514 |
| | | buffer seq | NA |
| | | polIII prom | 494 |
| | | buffer | NA |
| | | scaffold | 691 |
| | | spacer | 462 |
| | | buffer | 796 |
| | | 3′ ITR | 424 |
| | 33, | 5′ ITR | 423 |
| | 76, 78, 80, 82, 84, | buffer seq | 788 |
| | 86, 88, 90, | promoter | 464 |
| | 91-103 | buffer seq + STALL site | 842 |
| | | kozak | NA |
| | | start codon | NA |
| | | 5′ NLS | 832 |
| | | 5′ linker | NA |
| | | CasX 491 Protein | 791 |
| | | 3′ NLS linker | NA |
| | | 3′ NLS | 792 |
| | | tag | 793 |
| | | linker-stop codon | NA |
| | | buffer seq | 794 |
| | | polyA | 514 |
| | | buffer seq | NA |
| | | polIII prom | 494 |
| | | buffer | NA |
| | | scaffold | 691 |
| | | spacer | 462 |
| | | buffer | 796 |
| | | 3′ITR | 424 |
| Silencing | 104-119, 138- | 5′ ITR | 423 |
| Backbone | 139, 140, | Buffer seq | 788 |
| Transgenes | 146, 147 | promoter | 464 |
| Set 2 | | buffer seq | 789 |
| | 104-119, | STALL site | 2983 |
| | 138-139 | | |
| | 146 | STALL site | 2984 |
| | 104-119, 138- | kozak | NA |
| | 139, 140, | start codon | NA |
| | 146, 147 | 5′ NLS | 838 |
| | | 5′ linker | NA |
| | | CasX 491 Protein | 791 |
| | | 3′ NLS linker | NA |
| | | 3′ NLS | 2985 |
| | | linker-stop codon | NA |
| | | buffer seq | 794 |
| | | polyA | 514 |
| | | buffer seq | NA |
| | | polIII prom | 661 |
| | | buffer | NA |
| | | scaffold | 698 |
| | | spacer | 2920 |
| | | buffer | 796 |
| | | 3′ITR | |
| 2xSTALL | 136 | 5′ ITR | 423 |
| Constructs | (Same as 32) | buffer seq | 788 |
| In Vivo + | | promoter | 464 |
| Decoy | | 5′ buffer seq + STALL site | 842 |
| gRNA | | kozak | NA |
| | | start codon | NA |
| | | 5′ NLS | 832 |
| | | 5′ linker | NA |
| | | CasX 491 Protein | 791 |
| | | 3′ NLS linker | NA |
| | | 3′ NLS | 792 |
| | | tag | 793 |
| | | linker-stop codon | NA |
| | | 3′ buffer seq + STALL site | 843 |
| | | polyA | 514 |
| | | buffer seq | NA |
| | | polIII prom | 494 |
| | | buffer | NA |
| | | scaffold | 691 |
| | | spacer | 462 |
| | | buffer | 796 |
| | | 3′ ITR | 424 |
| | 137 | 5′ buffer seq + STALL site | 2986 |
| | | 3′ buffer seq + STALL site | 2987 |
| | 121-125, | 5′ buffer seq + STALL site | 2988 |
| | 128-135 | 3′ buffer seq + STALL site | 2989 |
| shRNA | 141-145 | pRepCap | 2990 |
| pRepCap | | | |
| shRNA | 167 | pRepCap | 4109 |
| pRepCap | | | |

Table 67 provides exemplary full-length siAAV constructs with one or two STALL sites. In Table 67, the spacer sequences of the sgRNA and the STALL sites are shown as wildcards that may indicate any nucleobase.

TABLE 67
DNA sequences of exemplary siAAV constructs
with one or two STALL sites

| | | SEQ ID NO (full-length |
|---|---|---|
| Number of STALL sites | STALL site PAM | construct sequence) |
| 1 | ATCN | 4151 |
| 1 | CTCN | 4152 |
| 1 | GTCN | 4153 |
| 2 | ATCN | 4154 |
| 2 | CTCN | 4155 |
| 2 | GTCN | 4156 |

1-87. (canceled)

88. A self-inactivating recombinant vector (SIRV) comprising a polynucleotide comprising:
a) one or more packaging components, wherein the packaging component comprises AAV 5′ and 3′ inverted terminal repeats (ITR);
b) a sequence encoding a Class 2 Type V protein comprising a single RNA-guided RuvC domain;
c) a first promoter operably linked to the sequence encoding the Class 2 Type V protein;
d) a sequence encoding a first guide RNA (gRNA) comprising a scaffold sequence linked to a targeting sequence that is complementary to and capable of hybridizing with: 1) a target nucleic acid of a cell to be modified; and 2) one or more self-inactivating segments incorporated in the polynucleotide;
e) a second promoter sequence operably linked to the sequence encoding the first gRNA; and
f) one or more self-inactivating segments comprising a protospacer adjacent motif (PAM) sequence and a polynucleotide sequence capable of being bound and cleaved by a ribonuclear protein complex (RNP) comprising the Class 2 Type V protein and the first gRNA,
wherein the one or more self-inactivating segments of the polynucleotide are located:
i) 5′ or 3′ adjacent to or within the sequence encoding the Class 2 Type V protein;
ii) 5′ or 3′ adjacent to or within a Kozak sequence located between the first promoter and the sequence encoding the Class 2 Type V protein;
iii) 5′ or 3′ adjacent to or within to the first promoter sequence;
iv) 5′ or 3′ adjacent to or within the second promoter sequence;
v) 3′ downstream of the transcriptional start site for the sequence encoding the Class 2 Type V protein;
vi) within one or more inserted introns in the polynucleotide encoding the Class 2 Type V protein;
vii) at the 3′ end of the polynucleotide encoding the Class 2 Type V protein, between a stop codon and poly(A) termination site for the Class2 Type V protein; or
viii) any combination of (i)-(vii); and
wherein the PAM sequence of the one or more self-inactivating segments:
1) is different by at least one nucleotide from the PAM sequence of the target nucleic acid of the cell to be modified; and
2) promotes less efficient cleavage or rate of cleavage of the self-inactivating segment by the RNP compared to the target nucleic acid of the cell to be modified; and
wherein cleavage by the RNP of the self-inactivating segment of the polynucleotide in a cell transduced or transfected with the SIRV results in reduced or eliminated expression of the Class 2 Type V protein or the gRNA encoded by the polynucleotide.

89. The SIRV of claim 88, wherein the self-inactivating segment comprises a sequence corresponding to any 15-21 nucleotide portion of the target nucleic acid sequence that is 3′ adjacent to a PAM sequence recognized by an RNP of the Class 2 Type V protein and the first gRNA.

90. The SIRV of claim 88, wherein:
a) if the PAM sequence of the target nucleic acid of the cell to be modified is TTC and the PAM preference of the Class 2 Type V protein is TTC, then the PAM sequence of the one or more self-inactivating segments is selected from the group consisting of ATC, CTC, TTT, GTT, and GTC;
b) if the PAM sequence of the target nucleic acid of the cell to be modified is ATC and the PAM preference of the Class 2 Type V protein is TTC, then the PAM sequence of the one or more self-inactivating segments is selected from the group consisting of CTC, TTT, GTT, and GTC;
c) if the PAM sequence of the target nucleic acid of the cell to be modified is CTC and the PAM preference of the Class 2 Type V protein is TTC, then the PAM sequence of the one or more self-inactivating segments is TTT, GTT, ATC, or GTC;
d) if the PAM sequence of the target nucleic acid of the cell to be modified is ATC and the PAM preference of the Class 2 Type V protein is ATC, then the PAM sequence of the one or more self-inactivating segments is selected from the group consisting of TTC, CTC, TTT, GTT, and GTC;
e) if the PAM sequence of the target nucleic acid of the cell to be modified is CTC and the PAM preference of the Class 2 Type V protein is ATC, then the PAM sequence of the one or more self-inactivating segments is selected from the group consisting of TTC, TTT, GTT, and GTC;
f) if the PAM sequence of the target nucleic acid of the cell to be modified is GTC and the PAM preference of the Class 2 Type V protein is ATC, then the PAM sequence of the one or more self-inactivating segments is TTC, TTT, CTC, or GTT;
g) if the PAM sequence of the target nucleic acid of the cell to be modified is GTC and the PAM preference of the Class 2 Type V protein is GTC, then the PAM sequence of the one or more self-inactivating segments is selected from the group consisting of ATC, CTC, TTT, GTT, and TTC;
h) if the PAM sequence of the target nucleic acid of the cell to be modified is GTC and the PAM preference of the Class 2 Type V protein is ATC, then the PAM sequence of the one or more self-inactivating segments is selected from the group consisting of TTC, TTT, GTT, and CTC;
i) if the PAM sequence of the target nucleic acid of the cell to be modified is CTC and the PAM preference of the Class 2 Type V protein is GTC, then the PAM sequence of the one or more self-inactivating segments is TTC, TTT, ATC, or GTT;
j) if the PAM sequence of the target nucleic acid of the cell to be modified is CTC and the PAM preference of the Class 2 Type V protein is CTC, then the PAM sequence of the one or more self-inactivating segments is selected from the group consisting of ATC, GTC, TTT, GTT, and TTC;
k) if the PAM sequence of the target nucleic acid of the cell to be modified is ATC and the PAM preference of the Class 2 Type V protein is CTC, then the PAM sequence of the one or more self-inactivating segments is selected from the group consisting of GTC, TTT, GTT, and TTC; or
l) if the PAM sequence of the target nucleic acid of the cell to be modified is GTC and the PAM preference of the Class 2 Type V protein is CTC, then the PAM sequence of the one or more self-inactivating segments is TTC, TTT, ATC, or GTT.

91. The SIRV of claim 88, wherein the one or more self-inactivating segments each have between about 1 to about 5 bases that are not complementary to corresponding positions in the targeting sequence of the first gRNA.

92. The SIRV of claim 88, wherein the percent cleavage by the RNP of the self-inactivating segments of the polynucleotide in a cell transfected or transduced with the SIRV is at least about 70%, less than the cleavage of the target nucleic acid in the cell in a timed in vitro cell-based assay, when assayed under comparable conditions, and wherein the time to achieve cleavage by the RNP of the self-inactivating segments of the polynucleotide in a cell transfected or transduced with the SIRV is delayed, relative to the time to achieve 90% editing of the target nucleic acid in the cell by at least about 9 days, when assayed in an in vitro assay under comparable conditions.

93. The SIRV of claim 88, wherein cleavage by the RNP of the self-inactivating segments of the polynucleotide in a cell transfected or transduced with the SIRV has a k_{cleave} rate that is at least about 2-fold less than the k_{cleave} rate of the target nucleic acid in an in vitro cell-based assay, when assayed under comparable conditions.

94. The SIRV of claim 88, wherein the Class 2 Type V protein further comprises one or more nuclear localization signals (NLS) located at or near the N-terminus and/or at or near the C-terminus of the Class 2 Type V protein.

95. The SIRV of claim 88, wherein the Class 2 Type V protein is a CasX protein selected from the group of sequences consisting of SEQ ID NOs: 1-3, 49-321 and 2356-2488, or a sequence having at least about 70% identity thereto.

96. The SIRV of claim 88, wherein the first gRNA has a scaffold comprising a sequence selected from the group of sequences consisting of SEQ ID NOS: 2101-2331, 3992-3995, and 4028 or a sequence having at least about 70% identity thereto, and wherein the first gRNA comprises a targeting sequence having 15 nucleotides, 16 nucleotides, 17, nucleotides, 18 nucleotides, 19 nucleotides, or 20 nucleotides.

97. A self-inactivating viral-derived particle comprising:
a) a viral capsid derived from an AAV serotype selected from the group consisting of AAV1, AAV2, AAV3, AAV4, AAV5, AAV6, AAV7, AAV8, AAV9, AAV10, AAV11, AAV12, AAV 44.9, AAV 9.45, AAV 9.61, AAV-Rh74, AAVRh10, and chimeras thereof;
b) 5′ and 3′ AAV ITR packaging components selected from the same serotype as the AAV capsid; and
c) the SIRV of claim 88.

98. A self-inactivating recombinant vector (SIRV) comprising a polynucleotide comprising:
a) one or more packaging components, wherein the packaging component is an AAV 5′ and 3′ inverted terminal repeat (ITR);
b) a sequence encoding a Class 2 Type V protein;
c) a first promoter operably linked to the sequence encoding the Class 2 Type V protein;
d) a sequence encoding a first guide RNA (gRNA) comprising a scaffold sequence and a linked targeting sequence that is complementary to a target nucleic acid of a cell to be modified;
e) a second promoter sequence operably linked to the sequence encoding the first gRNA;
and one or more of:
f) a sequence encoding a second gRNA comprising a targeting sequence complementary to both a target nucleic acid of a cell to be modified and to one or more self-inactivating segments of the SIRV, wherein the second gRNA comprises a scaffold sequence identical to the scaffold sequence of the first gRNA, wherein:
1) the sequence of the one or more self-inactivating segments is different by one or more nucleotides from the sequence of the target nucleic acid of the cell to be modified and promotes less efficient cleavage or rate of cleavage of the self-inactivating segment by the RNP compared to the target nucleic acid of the cell to be modified; and
2) the targeting sequence of the second gRNA is complementary to different or overlapping regions of the target nucleic acid sequence compared to the targeting sequence of the first gRNA;
g) a sequence encoding a second gRNA comprising a targeting sequence complementary to the one or more self-inactivating segments, the second gRNA comprising a scaffold sequence different from the scaffold sequence of the first gRNA, wherein the second gRNA promotes less efficient editing and/or cleavage by an RNP comprising the Class 2 Type V protein and the second gRNA compared to an RNP comprising the Class 2 Type V protein and the first gRNA;
h) a sequence encoding a second gRNA comprising a targeting sequence complementary to both a target nucleic acid of a cell to be modified and to one or more self-inactivating segments of the SIRV, wherein the second gRNA comprises a scaffold sequence identical to the scaffold sequence of the first gRNA, wherein:
1) the PAM sequence of the one or more self-inactivating segments is different by at least one nucleotide from the PAM sequence of the target nucleic acid of the cell to be modified and promotes less efficient cleavage or rate of cleavage of the self-inactivating segment by the RNP compared to the target nucleic acid of the cell to be modified; and
2) the targeting sequence of the second gRNA is complementary to different or overlapping regions of the target nucleic acid sequence compared to the targeting sequence of the first gRNA;
and/or
i) a third promoter sequence operably linked to the sequence encoding the second gRNA, wherein the third promoter sequence is different from the second promoter sequence and is less efficient at initiating transcription of the second gRNA compared to the second promoter initiating transcription of the first gRNA;
wherein the polynucleotide comprises one or more self-inactivating segments comprising a protospacer adjacent motif (PAM) sequence and a polynucleotide sequence capable of being bound and cleaved by a ribonuclear protein complex (RNP) of the Class 2 Type V protein and the second gRNA, and
wherein the one or more self-inactivating segments of the polynucleotide are located:
1) 5′ or 3′ adjacent to or within the sequence encoding the Class 2 Type V protein;
2) 5′ or 3′ adjacent to or within a Kozak sequence located between the first promoter and the sequence encoding the Class 2 Type V protein;
3) 5′ or 3′ adjacent to or within to the first promoter sequence;
4) 5′ or 3′ adjacent to or within the second promoter sequence;
5) 5′ or 3′ adjacent to or within the third promoter sequence;
6) 3′ downstream of the transcriptional start site for the sequence encoding the Class 2 Type V protein;
7) within one or more inserted introns in the sequence encoding the Class 2 Type V protein;
8) at the 3′ end of the polynucleotide encoding the Class 2 Type V protein, between a stop codon and poly(A) termination site of the sequence encoding the Class 2 Type V; or
9) in any combination of (1)-(8);
and wherein cleavage of the self-inactivating segments in a cell transduced or transfected with the SIRV by the RNP of the Class 2 Type V protein and the second gRNA results in reduced or eliminated expression of the Class 2 Type V protein or the gRNA encoded by the polynucleotide.

99. The SIRV of claim 98, comprising components (a)-(f), and (i).

100. The SIRV of claim 98, comprising components (a)-(e), (g), and (i).

101. The SIRV of claim 98, comprising components (a)-(e), (h) and (i).

102. The SIRV of claim 98, wherein the self-inactivating segment comprises a 15-21 nucleotide sequence complementary to the targeting sequence of the second gRNA and that is 3′ adjacent to a PAM sequence recognized by an RNP of the Class 2 Type V protein and the second gRNA.

103. The SIRV of claim 98, wherein the PAM sequence of the one or more self-inactivating segments:
a) is different from the PAM sequence of the target nucleic acid of the cell to be modified; and
b) promotes less efficient cleavage or rate of cleavage of the self-inactivating segment by the RNP of the Class 2 Type V protein and the second gRNA compared to the PAM of the target nucleic acid of the cell to be modified.

104. The SIRV of claim 103, wherein:
a) if the PAM sequence of the target nucleic acid of the cell to be modified is TTC and the PAM preference of the Class 2 Type V protein is TTC, then the PAM sequence of the one or more self-inactivating segments is selected from the group consisting of ATC, CTC, TTT, GTT, and GTC;
b) if the PAM sequence of the target nucleic acid of the cell to be modified is ATC and the PAM preference of the Class 2 Type V protein is TTC, then the PAM sequence of the one or more self-inactivating segments is selected from the group consisting of CTC, TTT, GTT, and GTC;
c) if the PAM sequence of the target nucleic acid of the cell to be modified is CTC and the PAM preference of the Class 2 Type V protein is TTC, then the PAM sequence of the one or more self-inactivating segments is GTC, TTT, ATC, or GTT;
d) if the PAM sequence of the target nucleic acid of the cell to be modified is ATC and the PAM preference of the Class 2 Type V protein is ATC, then the PAM sequence of the one or more self-inactivating segments is selected from the group consisting of TTC, CTC, TTT, GTT, and GTC;
e) if the PAM sequence of the target nucleic acid of the cell to be modified is CTC and the PAM preference of the Class 2 Type V protein is ATC, then the PAM sequence of the one or more self-inactivating segments is selected from the group consisting of TTC, TTT, GTT, and GTC;
f) if the PAM sequence of the target nucleic acid of the cell to be modified is GTC and the PAM preference of the Class 2 Type V protein is ATC, then the PAM sequence of the one or more self-inactivating segments is TTC, TTT, CTC, or GTT;
g) if the PAM sequence of the target nucleic acid of the cell to be modified is GTC and the PAM preference of the Class 2 Type V protein is GTC, then the PAM sequence of the one or more self-inactivating segments is selected from the group consisting of ATC, CTC, TTT, GTT, and TTC;
h) if the PAM sequence of the target nucleic acid of the cell to be modified is GTC and the PAM preference of the Class 2 Type V protein is ATC, then the PAM sequence of the one or more self-inactivating segments is selected from the group consisting of TTC, TTT, GTT, and CTC;
i) if the PAM sequence of the target nucleic acid of the cell to be modified is CTC and the PAM preference of the Class 2 Type V protein is GTC, then the PAM sequence of the one or more self-inactivating segments is TTC, TTT, ATC, or GTT;
j) if the PAM sequence of the target nucleic acid of the cell to be modified is CTC and the PAM preference of the Class 2 Type V protein is CTC, then the PAM sequence of the one or more self-inactivating segments is selected from the group consisting of ATC, GTC, TTT, GTT, and TTC;
k) if the PAM sequence of the target nucleic acid of the cell to be modified is ATC and the PAM preference of the Class 2 Type V protein is CTC, then the PAM sequence of the one or more self-inactivating segments is selected from the group consisting of GTC, TTT, GTT, and TTC; or
l) if the PAM sequence of the target nucleic acid of the cell to be modified is GTC and the PAM preference of the Class 2 Type V protein is CTC, then the PAM sequence of the one or more self-inactivating segments is TTC, TTT, ATC, or GTT.

105. The SIRV of claim 98, wherein the one or more self-inactivating segment sequences each have between 1 to 5 bases that are not complementary to corresponding positions in the targeting sequence of the second gRNA.

106. The SIRV of claim 98, wherein the RNP of the Class 2 Type V protein and second gRNA exhibit less efficient cleavage of the self-inactivating segment compared to the cleavage of the target nucleic acid of the cell by the RNP of the Class 2 Type V protein and first gRNA.

107. The SIRV of claim 98, wherein the Class 2 Type V protein further comprises one or more nuclear localization signals (NLS).

108. The SIRV of claim 98, wherein the Type V protein is a CasX protein selected from the group consisting of SEQ ID NOs: 1-3 and 49-321 and 2356-2488, or a sequence having at least about 70% sequence identity thereto, wherein the CasX protein is capable of forming a ribonuclear protein complex (RNP) with the first gRNA and the second gRNA upon expression in a cell transduced or transfected with the SIRV, and wherein the RNP of the CasX protein and the first gRNA is capable of cleaving the target nucleic acid and wherein the RNP of the CasX protein and the second gRNA is capable of cleaving the self-inactivating segment, and wherein the RNP of the CasX protein and the second gRNA exhibit a cleavage rate of the self-inactivating segments that is less efficient compared to the cleavage or rate of cleavage of the target nucleic acid by an RNP of the CasX protein and the first gRNA.

109. The SIRV of claim 98, wherein the second guide comprises a sequence selected from the group consisting of SEQ ID NO: 2101-2238 and the first guide comprises a sequence selected from the group consisting of SEQ ID NOS: 2276-2296.

110. The SIRV of claim 98, wherein the first and second gRNA each comprise a targeting sequence having 15 nucleotides, 16 nucleotides, 17, nucleotides, 18 nucleotides, 19 nucleotides, or 20 nucleotides.

111. A self-inactivating viral-derived particle comprising:
a) a viral capsid derived from an AAV serotype selected from the group consisting of AAV1, AAV2, AAV3, AAV4, AAV5, AAV6, AAV7, AAV8, AAV9, AAV10, AAV11, AAV12, AAV 44.9, AAV 9.45, AAV 9.61, AAV-Rh74, AAVRh10, and chimeras thereof;
b) 5′ and 3′ AAV ITR packaging components selected from the same serotype as the AAV capsid; and
c) the SIRV of claim 98.

112. A polynucleotide encoding an SIRV, wherein the polynucleotide comprises a sequence selected from the group consisting of SEQ ID NOs 4151-4156, or a sequence having at least about 70% sequence identity thereto.

113. A method of modifying a target nucleic acid sequence in a cell, comprising transfecting the cell with a SIRV comprising a polynucleotide comprising:
a) one or more packaging components, wherein the packaging component comprises AAV 5′ and 3′ inverted terminal repeats (ITR);
b) a sequence encoding a Class 2 Type V protein comprising a single RNA-guided RuvC domain;
c) a first promoter operably linked to the sequence encoding the Class 2 Type V protein;
d) a sequence encoding a first guide RNA (gRNA) comprising a scaffold sequence linked to a targeting sequence that is complementary to and capable of hybridizing with: 1) a target nucleic acid of a cell to be modified; and 2) one or more self-inactivating segments incorporated in the polynucleotide;
e) a second promoter sequence operably linked to the sequence encoding the first gRNA; and
f) one or more self-inactivating segments comprising a protospacer adjacent motif (PAM) sequence and a polynucleotide sequence capable of being bound and cleaved by a ribonuclear protein complex (RNP) comprising the Class 2 Type V protein and the first gRNA,
wherein the target nucleic acid sequence is modified by an RNP of the expressed Class 2 Type V protein and the first gRNA; and wherein the one or more self-inactivating segments of the polynucleotide are located:
1) 5′ or 3′ adjacent to or within the sequence encoding the Class 2 Type V protein;
2) 5′ or 3′ adjacent to or within a Kozak sequence located between the first promoter and the sequence encoding the Class 2 Type V protein;
3) 5′ or 3′ adjacent to or within to the first promoter sequence;
4) 5′ or 3′ adjacent to or within the second promoter sequence;
5) 3′ downstream of the transcriptional start site for the sequence encoding the Class 2 Type V protein;
6) within one or more inserted introns in the polynucleotide encoding the Class 2 Type V protein;
7) at the 3′ end of the polynucleotide encoding the Class 2 Type V protein, between a stop codon and poly(A) termination site for the Class2 Type V protein; or
8) any combination of (g)-(m), and
wherein the PAM sequence of the one or more self-inactivating segments:
i) is different by at least one nucleotide from the PAM sequence of the target nucleic acid of the cell to be modified; and
ii) promotes less efficient cleavage or rate of cleavage of the self-inactivating segment by the RNP compared to the target nucleic acid of the cell to be modified, and
wherein cleavage by the RNP of the self-inactivating segment of the polynucleotide in a cell transduced or transfected with the SIRV results in reduced or eliminated expression of the Class 2 Type V protein or the gRNA encoded by the polynucleotide.

114. The method of claim 113, wherein the modifying comprises introducing a single-stranded break or a double-stranded break in the target nucleic acid sequence of the cell, or wherein the modifying comprises introducing an insertion, deletion, or mutation in the target nucleic acid sequence of the cell, wherein the self-inactivating segment of the SIRV is cleaved by an RNP of the Class 2 Type V protein and the first gRNA subsequent to the modifying of the target nucleic acid sequence of the cell, wherein the cleavage of the self-inactivating segment results in reduced off-target modifying of a nucleic acid sequence in the cell compared to a cell transduced with an SIRV not comprising the self-inactivating segments, and wherein the cleavage of the self-inactivating segment results in reduced or eliminated expression of the Class 2 Type V protein in the cell.

115. The method of claim 114, wherein the self-inactivating segment is cleaved at least about 9 days after the modifying of the target nucleic acid sequence.

116. A composition comprising:
a) an AAV expression cassette; and
b) a polynucleotide comprising sequences encoding one or more small hairpin RNA (shRNA) sequences, each operably linked to a promoter, wherein the polynucleotide comprising the shRNA and linked promoters are linked exterior to the AAV transgene incorporated into a bacterial plasmid backbone,
wherein the AAV expression cassette comprises:
(i) a first adeno-associated virus (AAV) inverted terminal repeat (ITR) sequence;
(ii) a second AAV ITR sequence;
(iii) a sequence encoding a Class 2 Type V protein having a single RNA-guided RuvC domain;
(iv) a first promoter operably linked to the sequence encoding the Class 2 Type V protein;
(v) a sequence encoding a first guide RNA (gRNA) comprising a scaffold sequence and a linked targeting sequence that is complementary to and capable of hybridizing with a target nucleic acid of a cell to be modified; and
(vi) a second promoter sequence operably linked to the sequence encoding the first gRNA;
wherein the Class 2, Type V protein and gRNA encoding sequences are capable of being transcribed in a packaging cell transfected with the AAV expression cassette.

117. The composition of claim 116, wherein the shRNA encoding sequence comprises a sequence selected from the group consisting of SEQ ID NOS: 2640-2687, or a sequence having at least 85% identity thereto.

118. The composition of claim 116, wherein the polynucleotide comprising the shRNA and linked promoters are inserted into
a) an AAV RepCap plasmid;
b) an AAV Helper plasmid; and/or
c) a separate vector.

119. The composition of claim 116, wherein the encoded Class 2, Type V protein comprises a sequence selected from the group consisting of SEQ ID NOS: 1-3, 49-321 and 2356-2488, or a sequence having at least 85% identity thereto.

120. The composition of claim 116, wherein the first gRNA comprises a scaffold sequence selected from the group of sequences consisting of SEQ ID NOS: 2101-2331, 3992-3995, and 4028 or a sequence having at least 85% identity thereto.

121. The composition of claim 116, wherein the shRNA is capable of being expressed and processed in a packaging cell transfected with the polynucleotide into a siRNA sequence complementary to and capable of hybridizing with an mRNA of the Class 2, Type V protein transcribed by the packaging cell, wherein the packaging cell is selected from the group consisting of baby hamster kidney (BHK), human embryonic kidney 293 (HEK293), HEK293T, NS0, SP2/0, YO myeloma cells, A549, P3X63 mouse myeloma cells, PER, PER.C6, NIH3T3, COS, HeLa, and Chinese hamster ovary (CHO), and wherein upon hybridization of the siRNA sequence to the mRNA of the Class 2, Type V protein, the Class 2, Type V protein mRNA is degraded such that expression of the Class 2, Type V protein is reduced or eliminated in the packaging cell.

122. The composition of claim 121, wherein expression of the Class 2, Type V protein is reduced by at least 70% compared to a transfected packaging cell not comprising the shRNA, when assayed in a timed in vitro assay under comparable conditions.

123. The composition of claim 116, wherein the AAV expression cassette comprises:
a) one or more self-inactivating segments comprising a protospacer adjacent motif (PAM) sequence and a polynucleotide sequence capable of being bound and cleaved by a ribonuclear protein complex (RNP) of the Class 2 Type V protein and a second gRNA;
b) a sequence encoding a second gRNA comprising a targeting sequence complementary to the self-inactivating segment; and
c) a third promoter operably linked to the second gRNA, wherein the one or more self-inactivating segments of the polynucleotide are located:
1) 5′ or 3′ adjacent to or within the sequence encoding the Class 2 Type V protein;
2) 5′ or 3′ adjacent to or within a Kozak sequence located between the first promoter and the sequence encoding the Class 2 Type V protein;
3) 5′ or 3′ adjacent to or within to the first promoter sequence;
4) 5′ or 3′ adjacent to or within the second promoter sequence;
5) 5′ or 3′ adjacent to or within the third promoter sequence;
6) 3′ downstream of the transcriptional start site for the sequence encoding the Class 2 Type V protein;
7) within one or more inserted introns in the polynucleotide encoding the Class 2 Type V protein;
8) at the 3′ end of the polynucleotide encoding the Class 2 Type V protein, between a stop codon and poly(A) termination site of the sequence encoding the Class 2 Type V protein; or
9) any combination of (a)-(h), and
wherein cleavage of the self-inactivating segments in a cell transfected with the composition by the RNP of the Class 2 Type V protein and the second gRNA results in reduced or eliminated expression of the Class 2 Type V protein or the gRNA encoded by the polynucleotide.

124. The composition of claim 123, wherein the second gRNA comprises a scaffold sequence selected from the group of sequences consisting of SEQ ID NOS: 2101-2331, 3992-3995, and 4028 or a sequence having at least 85% identity thereto.

125. The composition of claim 123, wherein the self-inactivating segment comprises a 15-21 nucleotide sequence complementary to the targeting sequence of the second gRNA and that is 3′ adjacent to a PAM sequence recognized by an RNP of the Class 2 Type V protein and the second gRNA.

126. The composition of claim 123, wherein the PAM sequence of the one or more self-inactivating segments promotes less efficient cleavage or rate of cleavage of the self-inactivating segment by the RNP of the Class 2 Type V protein and the second gRNA compared to the PAM sequence 5′ and adjacent to the target nucleic acid of the cell to be modified.

127. A method for reducing premature cleavage of a self-inactivating AAV (siAAV) transgene encoding a Class 2 Type V nuclease protein and one or more gRNAs in a packaging cell, comprising introducing a polynucleotide sequence encoding one or more small hairpin RNA (shRNA) and linked promoters into the packaging cell comprising the siAAV transgene, wherein the polynucleotide comprising the shRNA and linked promoters are linked exterior to the AAV transgene inserted into a bacterial plasmid backbone, and wherein the shRNA is capable of being expressed and processed into an siRNA sequence, and
wherein the siRNA sequence is complementary to an mRNA of the Class 2 Type V nuclease transcribed by the packaging cell,
wherein the packaging cell is selected from the group consisting of BHK, HEK293, HEK293T, NS0, SP2/0, YO myeloma cells, A549, P3X63 mouse myeloma cells, PER, PER.C6, NIH3T3, COS, HeLa, and CHO, and
wherein the transgene comprises:
a) a first adeno-associated virus (AAV) inverted terminal repeat (ITR) sequence;
b) a second AAV ITR sequence;
c) a sequence encoding a Class 2 Type V protein having a single RNA-guided RuvC domain;
d) a first promoter operably linked to the sequence encoding the Class 2 Type V protein;
e) a sequence encoding a first guide RNA (gRNA) comprising a scaffold sequence and a linked targeting sequence that is complementary to and capable of hybridizing with a target nucleic acid of a cell to be modified; and
f) a second promoter sequence operably linked to the sequence encoding the first gRNA
g) a sequence encoding a second guide RNA (gRNA) comprising a scaffold sequence and a linked targeting sequence complementary to one or more self-inactivating segments of the transgene;
h) a third promoter sequence operably linked to the sequence encoding the second gRNA, wherein the third promoter has a sequence different from the sequence of the second promoter; and
i) one or more self-inactivating segments of the polynucleotide comprising a protospacer adjacent motif (PAM) sequence and a polynucleotide sequence capable of being bound and cleaved by a ribonuclear protein complex (RNP) of the Class 2 Type V protein and the second gRNA.

128. The method of claim 127, wherein the shRNA encoding sequence comprises a sequence selected from the group consisting of SEQ ID NOS: 2640-2687, or a sequence having at least 85% identity thereto.

129. The method of claim 128, wherein the polynucleotide comprising the shRNA and linked promoters are inserted into;
a) an AAV RepCap plasmid;
b) an AAV Helper plasmid; and/or
c) a separate vector.

130. The method of claim 127, wherein upon transcription of the shRNA and Class 2 Type V nuclease sequences, the shRNA is processed into siRNA which hybridizes with the mRNA of the Class 2 Type V nuclease and is degraded by the packaging cell.

131. The method of claim 130, wherein expression of the Class 2 Type V nuclease protein in the packaging cell is repressed by at least 70% compared to a transfected packaging cell not comprising the shRNA sequence, when assayed in a timed in vitro assay under comparable conditions.

132. The method of claim 127, wherein the Class 2 Type V nuclease protein is a CasX comprising a sequence of SEQ ID NO: 145, or a sequence having at least 85% identity thereto.

133. The method of claim 127, wherein the first and second gRNA each have a scaffold comprising a sequence of SEQ ID NO: 2296, or a sequence having at least about 70% sequence identity thereto.

134. The method of claim 127, wherein the second guide comprises a sequence of SEQ ID NO: 2238 and the first guide comprises a sequence of SEQ ID NO: 2296.
